(19) United States
(12) Patent Application Publication
(10) Pub. No.: US 2014/0045728 A1
(43) Pub. Date: Feb. 13, 2014

(54) Orthogonal Amplification and Assembly of Nucleic Acid Sequences

(71) Applicants: George M. Church, Brookline, MA (US); Sriram Kosuri, Cambridge, MA (US); Nikolai Eroshenko, Boston, MA (US)

(72) Inventors: George M. Church, Brookline, MA (US); Sriram Kosuri, Cambridge, MA (US); Nikolai Eroshenko, Boston, MA (US)

(73) Assignee: PRESIDENT AND FELLOWS OF HARVARD COLLEGE, Cambridge, MA (US)

(21) Appl. No.: 13/880,824

(22) PCT Filed: Oct. 20, 2011
(86) PCT No.: PCT/US11/57075

§ 371 Date: Oct. 11, 2013

Related U.S. Application Data
(60) Provisional application No. 61/405,801, filed on Oct. 22, 2010.

(51) Int. Cl. C12N 15/10

(52) U.S. Cl. CPC C12N 15/1031
USPC 506/26; 506/16

(57) ABSTRACT

Methods and compositions for synthesizing nucleic acid sequences of interest from heterogeneous mixtures of oligonucleotide sequences are provided.

### RELATED APPLICATION DATA

This application claims priority to U.S. Provisional Patent Application No. 61/405,801 filed on Oct. 22, 2010 and is hereby incorporated herein by reference in its entirety for all purposes.

### STATEMENT OF GOVERNMENT INTERESTS

This invention was made with government support under N000141010144 awarded by the Office of Naval Research, FG02-02ER63445 awarded by the department of Energy, W911NF-08-1-0254 awarded by the Defense Advanced Research Projects Agency, and HG003170 awarded by the National Institutes of Health. The government has certain rights in the invention.

### BACKGROUND

1. Field of the Invention

Embodiments of the present invention relate in general to methods and compositions for amplifying and assembling nucleic acid sequences.

2. Description of Related Art

The development of inexpensive, high-throughput and reliable gene synthesis methods will broadly stimulate progress in biology and biotechnology (Carr & Church (2009) Nat. Biotechnol. 27:1151). Currently, the reliance on column-synthesized oligonucleotides as a source of DNA limits further cost reductions in gene synthesis (Tian et al. (2009) Mol. BioSyst. 5:714). Oligonucleotides from DNA microchips can reduce costs by at least an order of magnitude, yet efforts to scale microchip use have been largely unsuccessful due to the high error rates and complexity of the oligonucleotide mixtures (Tian et al. (2004) Nature 432:1050; Richmond et al. (2004) Nucleic Acids Res. 32:5011; Zhou et al. (2004) Nucleic Acids Res. 32:5409).

The synthesis of novel DNA encoding regulatory elements, genes, pathways, and entire genomes provides powerful ways to both test biological hypotheses as well as harness biology for humankind's use. For example, since the initial use of oligonucleotides in deciphering the genetic code, DNA synthesis has engendered tremendous progress in biology with the recent complete synthesis of a viable bacterial genome (Nirenberg et al. (1961) Proc. Natl. Acad. Sci. USA 47:1588; Söll et al. (1965) Proc. Natl. Acad. Sci. USA 54:1378; Gibson et al. (2010) Science 329:52). Currently, almost all DNA synthesis relies on the use of phosphoramidite chemistry on controlled-pore glass (CPG) substrates. CPG oligonucleotides synthesized in this manner are effectively limited to approximately 100 bases by the yield and accuracy of the process. Thus, the synthesis of gene-sized fragments relies on assembling many oligonucleotides together using a variety of techniques termed gene synthesis (Tian (2009) (supra); Gibson (supra); Gibson (2009) Nucleic Acids Res. 37:6984; Li & Elledge (2007) Nat. Methods 4:251; Bang & Church (2008) Nat. Methods 5:37; Shao et al. (2009) Nucleic Acids Res. 37:e16).

The price of gene synthesis has reduced drastically over the last decade as the process has become increasingly industrialized. However, the current commercial price of gene synthesis, approximately $0.40-1.00/bp, has begun to approach the relatively stable cost of the CPG oligonucleotide precursors (approximately $0.10-0.20/bp) (Can (supra)). At these prices, the construction of large gene libraries and synthetic genomes is out of reach to most. To achieve further cost reductions, many current efforts focus on smaller volume synthesis of oligonucleotides in order to minimize reagent costs. For example, microfluidic oligonucleotide synthesis can reduce reagent cost by an order of magnitude (Lee et al. (2010) Nucleic Acids Res. 38:2514).

Another route is to harness existing DNA microchips, which can produce up to a million different oligonucleotides on a single chip, as a source of DNA for gene synthesis. Previous efforts have demonstrated the ability to synthesize genes from DNA microchips. Tian et al. described the assembly of 14.6 kb of novel DNA from 292 oligonucleotides synthesized on an Atactic/Xeotron chip (Tian (2004) (supra)). The process involved using 584 short oligonucleotides synthesized on the same chip for hybridization-based error correction. The resulting error rates were approximately 1/160 basepairs (bp) before error correction and approximately 1/1400 bp after. Using similar chips, Zhou et al. constructed approximately 12 genes with an error rate as low as 1/625 bp (Zhou (supra)). Richardson et al. showed the assembly of an 180 bp construct from eight oligonucleotides synthesized on a microarray using maskless photolithographic deprotection (Nimblegen) (Richmond (supra)). Though the error rates were not determined in that study, a follow-up construction of a 742 bp green fluorescent protein (GFP) sequence using the same process showed an error rate of 1/20 bp- 1/70 bp (Kim et al. (2006) Microelectronic Eng. 83:1613). These approaches have thus far failed to scale for at least two reasons. First, the error rates of chip-based oligonucleotides from DNA microchips are higher than traditional column-synthesized oligonucleotides. Second, the assembly of gene fragments becomes increasingly difficult as the diversity of the oligonucleotide mixture becomes larger.

### SUMMARY

The present invention provides methods and compositions to enrich one or more oligonucleotide sequences (e.g., DNA and/or RNA sequences) and assemble large nucleic acid sequences of interest (e.g., DNA and/or RNA sequences (e.g., genes, genomes and the like)) from complex mixtures of oligonucleotide sequences. The present invention further provides methods for generating oligonucleotide primers (e.g., orthogonal primers) that are useful for synthesizing one or more nucleic acid sequences of interest (e.g., gene(s), genome(s) and the like).

In certain exemplary embodiments, microarrays including at least 5,000 different oligonucleotide sequences are provided. Each oligonucleotide sequence of the microarray is a member of one of a plurality of oligonucleotide sets, and each oligonucleotide set is specific for a nucleic acid sequence of interest (e.g., a single nucleic acid sequence of interest). Each oligonucleotide sequence that is a member of a particular oligonucleotide set includes a pair of orthogonal primer binding sites having a sequence that is unique to said oligonucleotide set. The nucleic acid sequence of interest is at least 500 nucleotides in length. In certain aspects, at least 50, at least 100, or more oligonucleotide sets are provided wherein each set is specific for a unique nucleic acid sequence of interest. In other aspects, the oligonucleotide sequence of interest is at least 1,000, at least 2,500, at least 5,000, or more nucleotides in length. In still other aspects, the nucleic acid sequence of interest is a DNA sequence, e.g., a regulatory element, a gene, a pathway and/or a genome. In still other aspects, the microarray includes at least 10,000 different oligonucleotide sequences attached thereto.

In certain exemplary embodiments, a microarray comprising at least 10,000 different oligonucleotide sequences attached thereto is provided. Each oligonucleotide sequence of the microarray is a member of one of at least 50 oligonucleotide sets, and each oligonucleotide set is specific for a nucleic acid sequence of interest. Each oligonucleotide sequence that is a member of a particular oligonucleotide set includes a pair of orthogonal primer binding sites having a sequence that is unique to said oligonucleotide set. Each nucleic acid sequence of interest is at least 2,500 nucleotides in length.

In certain exemplary embodiments, methods of synthesizing a nucleic acid sequence of interest are provided. The methods include the steps of providing at least 5,000 different oligonucleotide sequences, wherein each oligonucleotide sequence is a member of one of a plurality of oligonucleotide sets, and each oligonucleotide set is specific for a nucleic acid sequences of interest. Each oligonucleotide sequence includes a pair of orthogonal primer binding sites having a sequence that is unique to a single oligonucleotide set. The methods includes the step of amplifying an oligonucleotide set using orthogonal primers that hybridize to the orthogonal primer binding sites unique to the set, and removing the orthogonal primer binding sites from the amplified oligonucleotide set. The methods further include the step of assembling the amplified oligonucleotide set into a nucleic acid sequence of interest that is at least 500 nucleotides in length. In certain aspects, the nucleic acid sequence of interest is at least 1,000, at least 2,500, at least 5,000, or more nucleotides in length. In other aspects, the nucleic acid sequence of interest is a DNA sequence, e.g., a regulatory element, a gene, a pathway and/or a genome. In yet other aspects, 50, 100, 500, 750, 1,000 or more oligonucleotide sets are provided, wherein each set is specific for a unique nucleic acid sequence of interest. In still other aspects, the 5,000 different oligonucleotide sequences are provided on a microarray and, optionally, the 5,000 different oligonucleotide sequences can be removed from the microarray prior to the step of amplifying.

Further features and advantages of certain embodiments of the present invention will become more fully apparent in the following description of the embodiments and drawings thereof, and from the claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The foregoing and other features and advantages of the present invention will be more fully understood from the following detailed description of illustrative embodiments taken in conjunction with the accompanying drawings in which:

FIGS. 1A-1F schematically depict scalable gene synthesis platform schematic for OLS Pool 2. Pre-designed oligonucleotides (no distinction is made between dsDNA and ssDNA in the figure) are synthesized on a DNA microchip (A) and then cleaved to make a pool of oligonucleotides (B). Plate-specific primer sequences (shades of yellow) are used to amplify separate plate subpools (C) (only two are shown), which contain DNA to assemble different genes (only three are shown for each plate subpool). Assembly specific sequences (shades of blue) are used to amplify assembly subpools (D) that contain only the DNA required to make a single gene. The primer sequences are cleaved (E) using either Type IIS restriction enzymes (resulting in dsDNA) or by DpnII/USER/λ exonuclease processing (producing ssDNA). Construction primers (shown as white and black sites flanking the full assembly) are then used in an assembly PCR reaction to build a gene from each assembly subpool (F). Depending on the downstream application the assembled products are then cloned either before or after an enzymatic error correction step.

FIGS. 2A-2D depict gene synthesis products. GFPmut3 was PCR assembled (A) from two different assembly subpools (GFP42 and GFP35) that were amplified from OLS Pool 1. Because the majority of the products were of the wrong size, the full-length assemblies were gel purified and re-amplified (B). Using the longer oligonucleotides in OLS Pool 2 a PCR assembly protocol was developed that did not require gel-isolation. This protocol was used to build three different fluorescent proteins (C). The building of 42 scFv regions that contained challenging GC-rich linkers was then attempted. Of the 42 assemblies (D), 40 resulted in strong bands of the correct size. The two that did not assemble (7 and 24) were gel isolated and re-amplified, resulting in bands of the correct size (see Supplementary FIG. 8b online). The antibody that corresponds to each number is given in Table 3. The sequences above each assembly represent the amino acid linker sequence used to link heavy and light chains in the scFv fragments.

FIGS. 3A-3B graphically depict products obtained from OLS Pool 1 and OLS Pool 2. The percentage of fluorescent cells resulting from synthesis products derived from column-synthesized oligonucleotides (black), OLS Chip 1 subpools GFP43 and GFP35 (green) and the three fluorescent proteins produced on OLS Chip 2 with and without ErrASE treatment (blue, yellow, and orange) are shown (A). The error bars correspond to the range of replicates from separate ligations. The error rates (average by of correct sequence per error) from various synthesis products are shown (B). Error bars show the expected Poisson error based on the number of errors found (±√n). Deletions of more than 2 consecutive bases are counted as a single error (no such errors were found in OLS Pool 1).

FIG. 4A-4B depict the amplification and processing of OLS Pool 1 oligonucleotides. Two assembly subpools and two control subpools were amplified from OLS Pool 1, which contained a total of 13,000 nucleotides (A). Because the oligonucleotides in the two GFP subpools had sizes distinct from all other nucleotides on the chip, and since no oligonucleotides of the incorrect size were detected, these data indicate that the oligonucleotides from other subpools did not amplify. The dsDNA subpools were then processed using DpnII/USER/lambda exonuclease (B). After processing, three types of products were obtained. First, there were the products of the expected size. Second, there were the high molecular weight products that corresponded to oligonucleotides that retained one or both of the assembly-specific primer sites. Last, there were the low molecular weight products that, without intending to be bound by scientific theory, were likely produced by DpnII cleavage at double stranded recognition sites formed by the overlapping regions of the oligonucleotides. The same dsDNA ladder (Low Molecular Weight, New England Biolabs, Ipswich, Mass.) was used in both the non-denaturing (A) and the denaturing (B) 10% PAGE gels, where the denaturing agent produced the extra bands in the ladder (B).

FIG. 5 depicts GFP assembly from OLS Pool 1. The two OLS Pool 1 GFP assembly subpools were amplified, processed and PCR assembled (See FIG. 3A). The bands corresponding to full-length assembly products were then gel-isolated and re-amplified. The re-amplification products shown contained low molecular weight products that, without intending to be bound by scientific theory, likely remained in trace amounts after gel isolation. These significantly greatly increased the number of clones that needed to be sequences in order to identify a perfect GFPmut3 construct. The control GFP was amplified from a cloned GFP. GFP20 was an assembly made from a hand mixed pool of oligonucleotides synthesized using a column-based method. GFP20 was not gel isolated or re-amplified.

FIGS. 6A-6C graphically depict screening error rates of GFP assemblies. Error rates from the first set (gel-isolated and re-amplified) (A), the second set (gel-isolated without re-amplification) (B), and the error-corrected second set of GFP assemblies from OLS Pool 1 (C) were determined using flow cytometry, by counting colonies on agar plates, and by sequencing individual clones. Error bars give the range of the observed values. n corresponds to the number of electroporated cultures from one or more ligation reactions performed on a single assembly reaction, with n=3-4 in (A) n=3 in (B), and n=2 in (C).

FIG. 7 graphically depicts the dynamic range of the flow cytometry screen. The relationship between the fluorescent fraction observed with flow cytometry is shown as a function of the fraction of perfect assemblies predicted from the sequencing data, with each data point corresponding to individual samples constructs built from the OLS Pool 1 (the same data are shown in FIG. 6). The black line indicates the result expected if the sequencing and fluorescent data predicted each other perfectly.

FIGS. 8A-8C depict processing of OLS 2 assembly subpools. Assembly-specific primers were used to amplify the subpools that were designed to build three different fluorescent proteins (A). A BtsI restriction enzyme was used to remove the priming sites (B). The same protocol was followed in processing the antibody assembly subpools, with (C) depicting the subpools after the BtsI digest. The gel in (C) depicts only 38 subpools because four antibody subpools evaporated from the reaction tubes during PCR, and had to be re-amplified in a separate experiment.

FIGS. 9A-9B graphically depict optimization of enzymatic synthesis error removal with ErrASE (Novici Biotech, Vacaville, Calif.). mCitrine synthesized from OLS Pool 2 was treated with ErrASE, and the fluorescent fraction was quantified with flow cytometry (A). The different ErrASE reactions corresponded to varying quantities of error-removing enzymes, with ErrASE 1 having the most and ErrASE 6 the least. Error bars give the range of the data points, with n=2 or 4 for the control and the mCitrine constructs, respectively. Increasing both the length of ErrASE treatment from 1 to 2 hours did not lead to a major decrease in error rates (B). “NO PRODUCT” indicates that the post-ErrASE amplification did not produce a product of the correct size. Without intending to be bound by scientific theory, this was most likely because the ErrASE error removing enzymes over-digested the assembly. Each value is an average of independent flow cytometry runs performed on five (A) or three (B) aliquots of the cloned assemblies.

FIGS. 10A-10I depict optimization of the antibody assembly protocol. First, each antibody assembly subpool was subjected to 15 PCR cycles in the presence of KOD DNA polymerase, but in the absence of construction primers. Next, the construction primers and each assembly was diluted in another PCR mix. Shown are the 2% agarose gels of the following assembly protocols: (A) KOD1; (B) KOD2; (C) KODXL60; (D) KODXL65; (E) Phusion62; (F) Phusion 67; (G) Phusion 72; (H) Phusion 62B; (I) Phusion67B. A 1 kb Plus DNA Ladder (Invitrogen, Carlsbad, Calif.) was used as a size marker in all experiments.

FIG. 11 depicts antibody assemblies that were screened. Here, eight of the 42 assembled scFv fragments were error-corrected with ErrASE, gel isolated, and re-amplified, generating the products shown. The constructs were subsequently cloned and sequenced (Table 3).

FIGS. 12A-12B depicts gels showing antibody assemblies. (A) The first assembly reaction resulted in 29 out of 42 antibody assembly reactions yielding products of the correct size. The antibody that corresponds to each number is listed in Table 3. Increasing the assembly subpool concentration used in the assembly reaction increased the number of successful assemblies to 40 (see FIG. 2D). The two failures from the second set of assembly reactions were gel-isolated and re-amplified, yielding products of the correct size (B).

FIGS. 13A-13B graphically depict the use of betaine during the ErrASE melt and re-anneal step. A set of synthesized antibodies (synthesis products shown in FIG. 2D) was treated with ErrASE, with betaine either included or left out of the melting and re-annealing step. The resulting error rate (A) and the probability of a synthesized molecule being either misassembled or having a large (3+ consecutive bp) deletion (B) was quantified. Error bars indicate the expected Poisson error (√n, with n being the number of errors observed).

FIG. 14 schematically depicts a full synthesis workflow according to certain aspects of the invention. The workflow was dependent on whether USER/DpnII processing (left branch after oligo synthesis) or type IIS enzymes (right branch) was used for removing the amplification sites. The process outlines a final optimized form of the optimized protocols. The times given in parentheses are estimates that account for both the time involved in setting up reactions and the time to complete the reaction.

FIG. 15 schematically depicts OLS Pool 1 assembly subpool amplification, and USER/DpnII processing. Assembly subpools were amplified from OLS Pool 1 using 20 bp priming sites that were shared by all primers in any particular assembly. A PCR reaction resulted in a pool of dsDNA with the oligos from other assemblies still in ssDNA form and at trace concentrations. The forward subpool amplification primers incorporates two sequential phosphorothioate linkages on the 5′ end, and a deoxyuridine its 3′ end, while the reverse primer had a phosphate at its 5′ end. Lambda exonuclease is a 5′ to 3′ exonuclease that degrades 5′ phosphorylated DNA and is blocked by phosphorothioate. This property was used to selectively degrade the remove strand of the amplified products. USER (Uracil-Specific Excision Reagent) Enzyme (New England Biolabs, Ipswich, Mass.) removed the 5′ priming site by excising the uracil and cutting 3′ and 5′ of the resulting apyrimidinic site. Next, the 3′ end was annealed to the reverse amplification primer, forming a double-stranded DpnII recognition site (5′ GATC). The 3′ priming site was then removed with a DpnII digest.

### DETAILED DESCRIPTION

The present invention is based in part on the discovery that high-fidelity DNA microchips, selective oligonucleotide amplification, optimized gene assembly protocols, and enzymatic error correction can be used to develop a highly parallel nucleic acid sequence (e.g., gene) synthesis platform. Assembly of 47 genes, including 42 challenging therapeutic antibody sequences, encoding a total of approximately 35 kilobasepairs of DNA has been surprisingly achieved using the compositions and methods described herein. These assemblies were created from a complex background containing 13,000 oligonucleotides encoding approximately 2.5 megabases of DNA, which is at least 50 times larger than previous attempts known in the art. A number of features were discovered to play an important role to the functionality of nucleic acid synthesis platform described herein, including the use of low-error starting material, well-chosen orthogonal primers, subpool amplification of individual assemblies, optimized assembly methods, and enzymatic error correction.

The present invention provides methods and compositions for the assembly of one or more nucleic acid sequences of interest from a large pool of oligonucleotide sequences. In certain exemplary embodiments, a nucleic acid sequence of interest is at least about 100, 200, 300, 400, 500 600, 700, 800, 900, 1,000, 1,500, 2,000, 2,500, 3,000, 3,500, 4,000, 4,500, 5,000, 5,500, 6,000, 6,500, 7,000, 7,500, 8,000, 8,500, 9,000, 9,500, 1,000,000, 2,000,000, 3,000,000, 4,000,000, 5,000,000, 6,000,000, 7,000,000, 8,000,000, 9,000,000, 10,000,000 or more nucleic acids in length. In other exemplary embodiments, a nucleic acid sequence of interest is between 100 and 10,000,000 nucleic acids in length, including any ranges therein. In yet other exemplary embodiments, a nucleic acid sequence of interest is between 100 and 20,000 nucleic acids in length, including any ranges therein. In still other exemplary embodiments, a nucleic acid sequence of interest is between 100 and 25,000 nucleic acids in length, including any ranges therein. In other aspects, a nucleic acid sequence of interest is a DNA sequence such as, e.g., a regulatory element (e.g., a promoter region, an enhancer region, a coding region, a non-coding region and the like), a gene, a genome, a pathway (e.g., a metabolic pathway (e.g., nucleotide metabolism, carbohydrate metabolism, amino acid metabolism, lipid metabolism, co-factor metabolism, vitamin metabolism, energy metabolism and the like), a signaling pathway, a biosynthetic pathway, an immunological pathway, a developmental pathway and the like) and the like. In yet other aspects, a nucleic acid sequence of interest is the length of a gene, e.g., between about 500 nucleotides and 5,000 nucleotides in length. In still other aspects, a nucleic acid sequence of interest is the length of a genome (e.g., a phage genome, a viral genome, a bacterial genome, a fungal genome, a plant genome, an animal genome or the like).

Embodiments of the present invention are directed to oligonucleotide sequences having two or more orthogonal primer binding sites that each hybridizes to an orthogonal primer. As used herein, the term “orthogonal primer binding site” is intended to include, but is not limited to, a nucleic acid sequence located at the 5′ and/or 3′ end of the oligonucleotide sequences of the present invention which hybridizes a complementary orthogonal primer. An “orthogonal primer pair” refers to a set of two primers of identical sequence that bind to both orthogonal primer binding sites at the 5′ and 3′ ends of each oligonucleotide sequence of an oligonucleotide set. Orthogonal primer pairs are designed to be mutually non-hybridizing to other orthogonal primer pairs, to have a low potential to cross-hybridize with one another or with oligonucleotide sequences, to have a low potential to form secondary structures, and to have similar melting temperatures (Tms) to one another. Orthogonal primer pair design and software useful for designing orthogonal primer pairs is discussed further herein.

As used herein, the term “oligonucleotide set” refers to a set of oligonucleotide sequences that has identical orthogonal pair primer sites and is specific for a nucleic acid sequence of interest. In certain aspects, a nucleic acid sequence of interest is synthesized from a plurality of oligonucleotide sequences that make up an oligonucleotide set. In other aspects, the plurality of oligonucleotide sequences that make up an oligonucleotide set are retrieved from a large pool of heterogeneous oligonucleotide sequences via common orthogonal primer binding sites. In certain aspects, an article of manufacture (e.g., a microchip, a test tube, a kit or the like) is provided that includes a plurality of oligonucleotide sequences encoding a mixture of oligonucleotide sets.

In certain exemplary embodiments, at least 100, 200, 300, 400, 500, 600, 700, 800, 900, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 21,000, 22,000, 23,000, 24,000, 25,000, 30,000, 35,000, 40,000, 45,000, 50,000, 55,000, 60,000, 65,000, 70,000, 75,000, 80,000, 85,000, 90,000, 95,000, 100,000 or more different oligonucleotide sequences are provided. In certain aspects, between about 2,000 and about 80,000 different oligonucleotide sequences are provided. In other aspects, between about 5,000 and about 60,000 different oligonucleotide sequences are provided. In still other aspects, about 55,000 different oligonucleotide sequences are provided.

In certain exemplary embodiments, the oligonucleotide sequences are at least 20, 30, 40, 50, 60, 70, 80, 90, 100, 110, 120, 130, 140, 150, 160, 170, 180, 190, 200, 210, 220, 230, 240, 250, 260, 270, 280, 290, 300, 350, 400, 450, 500, 550, 600, 650, 700, 750, 800, 850, 900, 950, 1000 or more nucleotides in length. In certain aspects, the oligonucleotide sequences are between about 50 and about 500 nucleotides in length. In other aspects, the oligonucleotide sequences are between about 100 and about 300 nucleotides in length. In other aspects, the oligonucleotide sequences are about 130 nucleotides in length. In still other aspects, the oligonucleotide sequences are about 200 nucleotides in length.

In certain exemplary embodiments, the oligonucleotide sequences encode at least 5, 10, 15, 20, 25, 30, 35, 40, 45, 50, 55, 60, 65, 70, 75, 80, 85, 90, 95, 100, 200, 300, 400, 500, 600, 700, 800, 900, 1,000, 2,000, 3,000, 4,000, 5,000, 6,000, 7,000, 8,000, 9,000, 10,000 or more different oligonucleotide sets.

In certain exemplary embodiments, at least 5, 10, 15, 20, 25, 30, 35, 40, 45, 50, 55, 60, 65, 70, 75, 80, 85, 90, 95, 100, 200, 300, 400, 500, 600, 700, 800, 900, 1,000, 2,000, 3,000, 4,000, 5,000, 6,000, 7,000, 8,000, 9,000, 10,000 different orthogonal primer pairs are provided.

In certain exemplary embodiments, assembly PCR is used to produce a nucleic acid sequence of interest from a plurality of oligonucleotide sequences that are members of a particular oligonucleotide set. “Assembly PCR” refers to the synthesis of long, double stranded nucleic acid sequences by performing PCR on a pool of oligonucleotides having overlapping segments. Assembly PCR is discussed further in Stemmer et al. (1995) Gene 164:49. In certain aspects, PCR assembly is used to assemble single stranded nucleic acid sequences (e.g., ssDNA) into a nucleic acid sequence of interest. In other aspects, PCR assembly is used to assemble double stranded nucleic acid sequences (e.g., dsDNA) into a nucleic acid sequence of interest.

In certain exemplary embodiments, methods are provided for designing a set of end-overlapping oligonucleotides for each nucleic acid sequence of interest (e.g., a gene, a regulatory element, a pathway, a genome or the like) that alternates on both the plus and minus strands and are useful for assembly PCR. In another aspect, oligonucleotide design is aided by a computer program, e.g. a computer program using algorithms as described herein.

In certain exemplary embodiments, various error correction methods are provided to remove errors in oligonucleotide sequences, subassemblies and/or nucleic acid sequences of interest. The term “error correction” refers to a process by which a sequence error in a nucleic acid molecule is corrected (e.g., an incorrect nucleotide at a particular location is changed to the nucleic acid that should be present based on the predetermined sequence). Methods for error correction include, for example, homologous recombination or sequence correction using DNA repair proteins.

The term “DNA repair enzyme” refers to one or more enzymes that correct errors in nucleic acid structure and sequence, i.e., recognizes, binds and corrects abnormal base-pairing in a nucleic acid duplex. Examples of DNA repair enzymes include, but are not limited to, proteins such as mutH, mutL, mutM, mutS, mutY, dam, thymidine DNA glycosylase (TDG), uracil DNA glycosylase, AlkA, MLH1, MSH2, MSH3, MSH6, Exonuclease I, T4 endonuclease V, Exonuclease V, RecJ exonuclease, FEN1 (RAD27), dnaQ (mutD), polC (dnaE), or combinations thereof, as well as homologs, orthologs, paralogs, variants, or fragments of the forgoing. In certain exemplary embodiments, the ErrASE system is used for error correction (Novici Biotech, Vacaville, Calif.). Enzymatic systems capable of recognition and correction of base pairing errors within the DNA helix have been demonstrated in bacteria, fungi and mammalian cells and the like.

Terms and symbols of nucleic acid chemistry, biochemistry, genetics, and molecular biology used herein follow those of standard treatises and texts in the field, e.g., Komberg and Baker, DNA Replication, Second Edition (W.H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); and the like.

“Complementary” or “substantially complementary” refers to the hybridization or base pairing or the formation of a duplex between nucleotides or nucleic acids, such as, for instance, between the two strands of a double stranded DNA molecule or between an oligonucleotide primer and a primer binding site on a single stranded nucleic acid. Complementary nucleotides are, generally, A and T (or A and U), or C and G. Two single-stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See Kanehisa (1984) Nucl. Acids Res. 12:203.

“Complex” refers to an assemblage or aggregate of molecules in direct or indirect contact with one another. In one aspect, “contact,” or more particularly, “direct contact,” in reference to a complex of molecules or in reference to specificity or specific binding, means two or more molecules are close enough so that attractive noncovalent interactions, such as van der Waal forces, hydrogen bonding, ionic and hydrophobic interactions, and the like, dominate the interaction of the molecules. In such an aspect, a complex of molecules is stable in that under assay conditions the complex is thermodynamically more favorable than a non-aggregated, or non-complexed, state of its component molecules. As used herein, “complex” refers to a duplex or triplex of polynucleotides or a stable aggregate of two or more proteins. In regard to the latter, a complex is formed by an antibody specifically binding to its corresponding antigen.

“Duplex” refers to at least two oligonucleotides and/or polynucleotides that are fully or partially complementary undergo Watson-Crick type base pairing among all or most of their nucleotides so that a stable complex is formed. The terms “annealing” and “hybridization” are used interchangeably to mean the formation of a stable duplex. In one aspect, stable duplex means that a duplex structure is not destroyed by a stringent wash, e.g., conditions including temperature of about 5° C. less that the Tₘ of a strand of the duplex and low monovalent salt concentration, e.g., less than 0.2 M, or less than 0.1 M. “Perfectly matched” in reference to a duplex means that the polynucleotide or oligonucleotide strands making up the duplex form a double stranded structure with one another such that every nucleotide in each strand undergoes Watson-Crick base pairing with a nucleotide in the other strand. The term “duplex” comprehends the pairing of nucleoside analogs, such as deoxyinosine, nucleosides with 2-aminopurine bases, PNAs, and the like, that may be employed. A “mismatch” in a duplex between two oligonucleotides or polynucleotides means that a pair of nucleotides in the duplex fails to undergo Watson-Crick bonding.

“Genetic locus,” or “locus” refers to a contiguous sub-region or segment of a genome. As used herein, genetic locus, or locus, may refer to the position of a nucleotide, a gene, or a portion of a gene in a genome, including mitochondrial DNA, or it may refer to any contiguous portion of genomic sequence whether or not it is within, or associated with, a gene. In one aspect, a genetic locus refers to any portion of genomic sequence, including mitochondrial DNA, from a single nucleotide to a segment of few hundred nucleotides, e.g. 100-300, in length. Usually, a particular genetic locus may be identified by its nucleotide sequence, or the nucleotide sequence, or sequences, of one or both adjacent or flanking regions. In another aspect, a genetic locus refers to the expressed nucleic acid product of a gene, such as an RNA molecule or a cDNA copy thereof.

“Hybridization” refers to the process in which two single-stranded polynucleotides bind non-covalently to form a stable double-stranded polynucleotide. The term “hybridization” may also refer to triple-stranded hybridization. The resulting (usually) double-stranded polynucleotide is a “hybrid” or “duplex.” “Hybridization conditions” will typically include salt concentrations of less than about 1 M, more usually less than about 500 mM and even more usually less than about 200 mM. Hybridization temperatures can be as low as 5° C., but are typically greater than 22° C., more typically greater than about 30° C., and often in excess of about 37° C. Hybridizations are usually performed under stringent conditions, i.e., conditions under which a probe will hybridize to its target subsequence. Stringent conditions are sequence-dependent and are different in different circumstances. Longer fragments may require higher hybridization temperatures for specific hybridization. As other factors may affect the stringency of hybridization, including base composition and length of the complementary strands, presence of organic solvents and extent of base mismatching, the combination of parameters is more important than the absolute measure of any one alone. Generally, stringent conditions are selected to be about 5° C. lower than the Tₙ, for the specific sequence at s defined ionic strength and pH. Exemplary stringent conditions include salt concentration of at least 0.01 M to no more than 1 M Na ion concentration (or other salts) at a pH 7.0 to 8.3 and a temperature of at least 25° C. For example, conditions of 5×SSPE (750 mM NaCl, 50 mM Na phosphate, 5 mM EDTA, pH 7.4) and a temperature of 25-30° C. are suitable for allele-specific probe hybridizations. For stringent conditions, see for example, Sambrook, Fritsche and Maniatis, Molecular Cloning A Laboratory Manual, 2nd Ed. Cold Spring Harbor Press (1989) and Anderson Nucleic Acid Hybridization, 1^{st} Ed., BIOS Scientific Publishers Limited (1999). “Hybridizing specifically to” or “specifically hybridizing to” or like expressions refer to the binding, duplexing, or hybridizing of a molecule substantially to or only to a particular nucleotide sequence or sequences under stringent conditions when that sequence is present in a complex mixture (e.g., total cellular) DNA or RNA.

“Kit” refers to any delivery system for delivering materials or reagents for carrying out a method of the invention. In the context of assays, such delivery systems include systems that allow for the storage, transport, or delivery of reaction reagents (e.g., primers, enzymes, microarrays, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials for assays of the invention. Such contents may be delivered to the intended recipient together or separately. For example, a first container may contain an enzyme for use in an assay, while a second container contains primers.

“Ligation” means to form a covalent bond or linkage between the termini of two or more nucleic acids, e.g., oligonucleotides and/or polynucleotides, in a template-driven reaction. The nature of the bond or linkage may vary widely and the ligation may be carried out enzymatically or chemically. As used herein, ligations are usually carried out enzymatically to form a phosphodiester linkage between a 5′ carbon of a terminal nucleotide of one oligonucleotide with 3′ carbon of another oligonucleotide. A variety of template-driven ligation reactions are described in the following references: Whitely et al., U.S. Pat. No. 4,883,750; Letsinger et al., U.S. Pat. No. 5,476,930; Fung et al., U.S. Pat. No. 5,593,826; Kool, U.S. Pat. No. 5,426,180; Landegren et al., U.S. Pat. No. 5,871,921; Xu and Kool (1999) Nucl. Acids Res. 27:875; Higgins et al., Meth. in Enzymol. (1979) 68:50; Engler et al. (1982) The Enzymes, 15:3 (1982); and Namsaraev, U.S. Patent Pub. 2004/0110213.

“Amplifying” includes the production of copies of a nucleic acid molecule of the array or a nucleic acid molecule bound to a bead via repeated rounds of primed enzymatic synthesis. “In situ” amplification indicated that the amplification takes place with the template nucleic acid molecule positioned on a support or a bead, rather than in solution. In situ amplification methods are described in U.S. Pat. No. 6,432,360.

“Support” can refer to a matrix upon which nucleic acid molecules of a nucleic acid array are placed. The support can be solid or semi-solid or a gel. “Semi-solid” refers to a compressible matrix with both a solid and a liquid component, wherein the liquid occupies pores, spaces or other interstices between the solid matrix elements. Semi-solid supports can be selected from polyacrylamide, cellulose, polyamide (nylon) and crossed linked agarose, dextran and polyethylene glycol.

“Randomly-patterned” or “random” refers to non-ordered, non-Cartesian distribution (in other words, not arranged at pre-determined points along the x- or y-axes of a grid or at defined “clock positions,” degrees or radii from the center of a radial pattern) of nucleic acid molecules over a support, that is not achieved through an intentional design (or program by which such design may be achieved) or by placement of individual nucleic acid features. Such a “randomly-patterned” or “random” array of nucleic acids may be achieved by dropping, spraying, plating or spreading a solution, emulsion, aerosol, vapor or dry preparation comprising a pool of nucleic acid molecules onto a support and allowing the nucleic acid molecules to settle onto the support without intervention in any manner to direct them to specific sites thereon. Arrays of the invention can be randomly patterned or random.

“Heterogeneous” refers to a population or collection of nucleic acid molecules that comprises a plurality of different sequences. According to one aspect, a heterogeneous pool of oligonucleotide sequences is provided with an article of manufacture (e.g., a microarray).

“Nucleoside” as used herein includes the natural nucleosides, including 2′-deoxy and 2′-hydroxyl forms, e.g. as described in Komberg and Baker, DNA Replication, 2nd Ed. (Freeman, San Francisco, 1992). “Analogs” in reference to nucleosides includes synthetic nucleosides having modified base moieties and/or modified sugar moieties, e.g., described by Scheit, Nucleotide Analogs (John Wiley, New York, 1980); Uhlman and Peyman, Chemical Reviews, 90:543-584 (1990), or the like, with the proviso that they are capable of specific hybridization. Such analogs include synthetic nucleosides designed to enhance binding properties, reduce complexity, increase specificity, and the like. Polynucleotides comprising analogs with enhanced hybridization or nuclease resistance properties are described in Uhlman and Peyman (cited above); Crooke et al., Exp. Opin. Ther. Patents, 6: 855-870 (1996); Mesmaeker et al., Current Opinion in Structural Biology, 5:343-355 (1995); and the like. Exemplary types of polynucleotides that are capable of enhancing duplex stability include oligonucleotide phosphoramidates (referred to herein as “amidates”), peptide nucleic acids (referred to herein as “PNAs”), oligo-2′-O-alkylribonucleotides, polynucleotides containing C-5 propynylpyrimidines, locked nucleic acids (LNAs), and like compounds. Such oligonucleotides are either available commercially or may be synthesized using methods described in the literature.

“Oligonucleotide” or “polynucleotide,” which are used synonymously, means a linear polymer of natural or modified nucleosidic monomers linked by phosphodiester bonds or analogs thereof. The term “oligonucleotide” usually refers to a shorter polymer, e.g., comprising from about 3 to about 100 monomers, and the term “polynucleotide” usually refers to longer polymers, e.g., comprising from about 100 monomers to many thousands of monomers, e.g., 10,000 monomers, or more. Oligonucleotides comprising probes or primers usually have lengths in the range of from 12 to 60 nucleotides, and more usually, from 18 to 40 nucleotides. Oligonucleotides and polynucleotides may be natural or synthetic. Oligonucleotides and polynucleotides include deoxyribonucleosides, ribonucleosides, and non-natural analogs thereof, such as anomeric forms thereof, peptide nucleic acids (PNAs), and the like, provided that they are capable of specifically binding to a target genome by way of a regular pattern of monomer-to-monomer interactions, such as Watson-Crick type of base pairing, base stacking, Hoogsteen or reverse Hoogsteen types of base pairing, or the like.

Usually nucleosidic monomers are linked by phosphodiester bonds. Whenever an oligonucleotide is represented by a sequence of letters, such as “ATGCCTG,” it will be understood that the nucleotides are in 5′ to 3′ order from left to right and that “A” denotes deoxyadenosine, “C” denotes deoxycytidine, “G” denotes deoxyguanosine, “T” denotes deoxythymidine, and “U” denotes the ribonucleoside, uridine, unless otherwise noted. Usually oligonucleotides comprise the four natural deoxynucleotides; however, they may also comprise ribonucleosides or non-natural nucleotide analogs. It is clear to those skilled in the art when oligonucleotides having natural or non-natural nucleotides may be employed in methods and processes described herein. For example, where processing by an enzyme is called for, usually oligonucleotides consisting solely of natural nucleotides are required. Likewise, where an enzyme has specific oligonucleotide or polynucleotide substrate requirements for activity, e.g., single stranded DNA, RNA/DNA duplex, or the like, then selection of appropriate composition for the oligonucleotide or polynucleotide substrates is well within the knowledge of one of ordinary skill, especially with guidance from treatises, such as Sambrook et al., Molecular Cloning, Second Edition (Cold Spring Harbor Laboratory, New York, 1989), and like references. Oligonucleotides and polynucleotides may be single stranded or double stranded.

“Polymorphism” or “genetic variant” means a substitution, inversion, insertion, or deletion of one or more nucleotides at a genetic locus, or a translocation of DNA from one genetic locus to another genetic locus. In one aspect, polymorphism means one of multiple alternative nucleotide sequences that may be present at a genetic locus of an individual and that may comprise a nucleotide substitution, insertion, or deletion with respect to other sequences at the same locus in the same individual, or other individuals within a population. An individual may be homozygous or heterozygous at a genetic locus; that is, an individual may have the same nucleotide sequence in both alleles, or have a different nucleotide sequence in each allele, respectively. In one aspect, insertions or deletions at a genetic locus comprises the addition or the absence of from 1 to 10 nucleotides at such locus, in comparison with the same locus in another individual of a population (or another allele in the same individual). Usually, insertions or deletions are with respect to a major allele at a locus within a population, e.g., an allele present in a population at a frequency of fifty percent or greater.

“Primer” includes an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process are determined by the sequence of the template polynucleotide. Usually primers are extended by a DNA polymerase. Primers usually have a length in the range of between 3 to 36 nucleotides, also 5 to 24 nucleotides, also from 14 to 36 nucleotides. Primers within the scope of the invention include orthogonal primers, amplification primers, constructions primers and the like. Pairs of primers can flank a sequence of interest or a set of sequences of interest. Primers and probes can be degenerate in sequence. Primers within the scope of the present invention bind adjacent to a target sequence (e.g., an oligonucleotide sequence of an oligonucleotide set or a nucleic acid sequence of interest).

In certain exemplary embodiments, orthogonal primers/primer binding sites are designed to be temporary, e.g., to permit removal of the orthogonal primers/primer binding sites at a desired stage prior to and/or during assembly. Temporary orthogonal primers/primer binding sites may be designed so as to be removable by chemical, thermal, light based, or enzymatic cleavage. Cleavage may occur upon addition of an external factor (e.g., an enzyme, chemical, heat, light, etc.) or may occur automatically after a certain time period (e.g., after n rounds of amplification). In one embodiment, temporary orthogonal primers/primer binding sites may be removed by chemical cleavage. For example, orthogonal primers/primer binding sites having acid labile or base labile sites may be used for amplification. The amplified pool may then be exposed to acid or base to remove the orthogonal primer/primer binding sites at the desired location. Alternatively, the temporary primers may be removed by exposure to heat and/or light. For example, orthogonal primers/primer binding sites having heat labile or photolabile sites may be used for amplification. The amplified pool may then be exposed to heat and/or light to remove the orthogonal primer/primer binding sites at the desired location. In another embodiment, an RNA primer may be used for amplification thereby forming short stretches of RNA/DNA hybrids at the ends of the nucleic acid molecule. The orthogonal primers/primer binding sites may then be removed by exposure to an RNase (e.g., RNase H). In various embodiments, the method for removing the primer may only cleave a single strand of the amplified duplex thereby leaving 3′ or 5′ overhangs. Such overhangs may be removed using an exonuclease to form blunt ended double stranded duplexes. For example, RecJ_{f} may be used to remove single stranded 5′ overhangs and Exonuclease I or Exonuclease T may be used to remove single stranded 3′ overhangs. Additionally, S₁ nuclease, P₁ nuclease, mung bean nuclease, and CEL I nuclease, may be used to remove single stranded regions from a nucleic acid molecule. RecJ_{f}, Exonuclease I, Exonuclease T, and mung bean nuclease are commercially available, for example, from New England Biolabs (Beverly, Mass.). S1 nuclease, P1 nuclease and CEL I nuclease are described, for example, in Vogt, V. M., Eur. J. Biochem., 33: 192-200 (1973); Fujimoto et al., Agric. Biol. Chem. 38: 777-783 (1974); Vogt, V. M., Methods Enzymol. 65: 248-255 (1980); and Yang et al., Biochemistry 39: 3533-3541 (2000).

In one embodiment, the temporary orthogonal primers/primer binding sites may be removed from a nucleic acid by chemical, thermal, or light based cleavage. Exemplary chemically cleavable internucleotide linkages for use in the methods described herein include, for example, β-cyano ether, 5′-deoxy-5′-aminocarbamate, 3′ deoxy-3′-aminocarbamate, urea, 2′ cyano-3′,5′-phosphodiester, 3′-(S)-phosphorothioate, 5′-(S)-phosphorothioate, 3′-(N)-phosphoramidate, 5′-(N)-phosphoramidate, α-amino amide, vicinal diol, ribonucleoside insertion, 2′-amino-3′,5′-phosphodiester, allylic sulfoxide, ester, silyl ether, dithioacetal, 5′-thio-furmal, α-hydroxy-methyl-phosphonic bisamide, acetal, 3′-thio-furmal, methylphosphonate and phosphotriester. Internucleoside silyl groups such as trialkylsilyl ether and dialkoxysilane are cleaved by treatment with fluoride ion. Base-cleavable sites include 3-cyano ether, 5′-deoxy-5′-aminocarbamate, 3′-deoxy-3′-aminocarbamate, urea, 2′-cyano-3′,5′-phosphodiester, 2′-amino-3′,5′-phosphodiester, ester and ribose. Thio-containing internucleotide bonds such as 3′-(S)-phosphorothioate and 5′-(S)-phosphorothioate are cleaved by treatment with silver nitrate or mercuric chloride. Acid cleavable sites include 3′-(N)-phosphoramidate, 5′-(N)-phosphoramidate, dithioacetal, acetal and phosphonic bisamide. An α-aminoamide internucleoside bond is cleavable by treatment with isothiocyanate, and titanium may be used to cleave a 2′-amino-3′,5′-phosphodiester-O-ortho-benzyl internucleoside bond. Vicinal diol linkages are cleavable by treatment with periodate. Thermally cleavable groups include allylic sulfoxide and cyclohexene while photo-labile linkages include nitrobenzylether and thymidine dimer. Methods synthesizing and cleaving nucleic acids containing chemically cleavable, thermally cleavable, and photo-labile groups are described for example, in U.S. Pat. No. 5,700,642.

In other embodiments, temporary orthogonal primers/primer binding sites may be removed using enzymatic cleavage. For example, orthogonal primers/primer binding sites may be designed to include a restriction endonuclease cleavage site. After amplification, the pool of nucleic acids may be contacted with one or more endonucleases to produce double stranded breaks thereby removing the primers/primer binding sites. In certain embodiments, the forward and reverse primers may be removed by the same or different restriction endonucleases. Any type of restriction endonuclease may be used to remove the primers/primer binding sites from nucleic acid sequences. A wide variety of restriction endonucleases having specific binding and/or cleavage sites are commercially available, for example, from New England Biolabs (Ipswich, Mass.). In various embodiments, restriction endonucleases that produce 3′ overhangs, 5′ overhangs or blunt ends may be used. When using a restriction endonuclease that produces an overhang, an exonuclease (e.g., RecJ_{f}, Exonuclease I, Exonuclease T, S₁ nuclease, P₁ nuclease, mung bean nuclease, CEL I nuclease, etc.) may be used to produce blunt ends. In an exemplary embodiment, an orthogonal primer/primer binding site that contains a binding and/or cleavage site for a type IIS restriction endonuclease may be used to remove the temporary orthogonal primer binding site

As used herein, the term “restriction endonuclease recognition site” is intended to include, but is not limited to, a particular nucleic acid sequence to which one or more restriction enzymes bind, resulting in cleavage of a DNA molecule either at the restriction endonuclease recognition sequence itself, or at a sequence distal to the restriction endonuclease recognition sequence. Restriction enzymes include, but are not limited to, type I enzymes, type II enzymes, type IIS enzymes, type III enzymes and type IV enzymes. The REBASE database provides a comprehensive database of information about restriction enzymes, DNA methyltransferases and related proteins involved in restriction-modification. It contains both published and unpublished work with information about restriction endonuclease recognition sites and restriction endonuclease cleavage sites, isoschizomers, commercial availability, crystal and sequence data (see Roberts et al. (2005) Nucl. Acids Res. 33:D230, incorporated herein by reference in its entirety for all purposes).

In certain aspects, primers of the present invention include one or more restriction endonuclease recognition sites that enable type HS enzymes to cleave the nucleic acid several base pairs 3′ to the restriction endonuclease recognition sequence. As used herein, the term “type IIS” refers to a restriction enzyme that cuts at a site remote from its recognition sequence. Type HS enzymes are known to cut at a distances from their recognition sites ranging from 0 to 20 base pairs. Examples of Type Hs endonucleases include, for example, enzymes that produce a 3′ overhang, such as, for example, Bsr I, Bsm I, BstF5 I, BsrD I, Bts I, Mnl I, BciV I, Hph I, Mbo II, Eci I, Acu I, Bpm I, Mme I, BsaX I, Bcg I, Bae I, Bfi I, TspDT I, TspGW I, Taq II, Eco57 I, Eco57M I, Gsu I, Ppi I, and Psr I; enzymes that produce a 5′ overhang such as, for example, BsmA I, Ple I, Fau I, Sap I, BspM I, SfaN I, Hga I, Bvb I, Fok I, BceA I, BsmF I, Ksp632 I, Eco31 I, Esp3 I, Aar I; and enzymes that produce a blunt end, such as, for example, Mly I and Btr I. Type-IIs endonucleases are commercially available and are well known in the art (New England Biolabs, Beverly, Mass.). Information about the recognition sites, cut sites and conditions for digestion using type Hs endonucleases may be found, for example, on the Worldwide web at neb.com/nebecomm/enzymefindersearch bytypeIIs.asp). Restriction endonuclease sequences and restriction enzymes are well known in the art and restriction enzymes are commercially available (New England Biolabs, Ipswich, Mass.).

Primers (e.g., orthogonal primers, amplification primers, construction primers and the like) suitable for use in the methods disclosed herein may be designed with the aid of a computer program, such as, for example, DNAWorks, Gene2Oligo, or using the parameters software described herein. Typically primers are from about 5 to about 500, about 10 to about 100, about 10 to about 50, or about 10 to about 30 nucleotides in length. In certain exemplary embodiments, a set of orthogonal primers or a plurality of sets of orthogonal primers are designed so as to have substantially similar melting temperatures to facilitate manipulation of a complex reaction mixture. The melting temperature may be influenced, for example, by primer length and nucleotide composition. In certain exemplary embodiments, a plurality of sets of orthogonal primers are designed such that each set of orthogonal primers is mutually non-hybridizing with one another. Methods for designing orthogonal primers are described further herein.

“Solid support,” “support,” and “solid phase support” are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations. Microarrays usually comprise at least one planar solid phase support, such as a glass microscope slide. Semisolid supports and gel supports are also useful in the present invention.

“Specific” or “specificity” in reference to the binding of one molecule to another molecule, such as a target sequence to a probe, means the recognition, contact, and formation of a stable complex between the two molecules, together with substantially less recognition, contact, or complex formation of that molecule with other molecules. In one aspect, “specific” in reference to the binding of a first molecule to a second molecule means that to the extent the first molecule recognizes and forms a complex with another molecules in a reaction or sample, it forms the largest number of the complexes with the second molecule. In certain aspects, this largest number is at least fifty percent. Generally, molecules involved in a specific binding event have areas on their surfaces or in cavities giving rise to specific recognition between the molecules binding to each other. Examples of specific binding include antibody-antigen interactions, enzyme-substrate interactions, formation of duplexes or triplexes among polynucleotides and/or oligonucleotides, receptor-ligand interactions, and the like. As used herein, “contact” in reference to specificity or specific binding means two molecules are close enough that weak non-covalent chemical interactions, such as van der Waal forces, hydrogen bonding, base-stacking interactions, ionic and hydrophobic interactions, and the like, dominate the interaction of the molecules.

“Spectrally resolvable” in reference to a plurality of fluorescent labels means that the fluorescent emission bands of the labels are sufficiently distinct, i.e., sufficiently non-overlapping, that molecular tags to which the respective labels are attached can be distinguished on the basis of the fluorescent signal generated by the respective labels by standard photodetection systems, e.g., employing a system of band pass filters and photomultiplier tubes, or the like, as exemplified by the systems described in U.S. Pat. Nos. 4,230,558; 4,811,218, or the like, or in Wheeless et al., pgs. 21-76, in Flow Cytometry Instrumentation and Data Analysis (Academic Press, New York, 1985). In one aspect, spectrally resolvable organic dyes, such as fluorescein, rhodamine, and the like, means that wavelength emission maxima are spaced at least 20 nm apart, and in another aspect, at least 40 nm apart. In another aspect, chelated lanthanide compounds, quantum dots, and the like, spectrally resolvable means that wavelength emission maxima are spaced at least 10 nm apart, and in a further aspect, at least 15 nm apart.

“Tₘ” is used in reference to “melting temperature.” Melting temperature is the temperature at which a population of double-stranded nucleic acid molecules becomes half dissociated into single strands. Several equations for calculating the Tₘ of nucleic acids are well known in the art. As indicated by standard references, a simple estimate of the Tₘ value may be calculated by the equation. Tₘ=81.5+0.41 (% G+C), when a nucleic acid is in aqueous solution at 1 M NaCl (see e.g., Anderson and Young, “Quantitative Filter Hybridization,” in Nucleic Acid Hybridization (1985). Other references (e.g., Allawi, H. T. & Santa Lucia, J., Jr., Biochemistry 36, 10581-94 (1997)) include alternative methods of computation which take structural and environmental, as well as sequence characteristics into account for the calculation of Tₘ.

In certain exemplary embodiments, oligonucleotide sequences are provided on a solid support. Oligonucleotide sequences may be synthesized on a solid support in an array format, e.g., a microarray of single stranded DNA segments synthesized in situ on a common substrate wherein each oligonucleotide is synthesized on a separate feature or location on the substrate. Arrays may be constructed, custom ordered, or purchased from a commercial vendor. Various methods for constructing arrays are well known in the art. For example, methods and techniques applicable to synthesis of construction and/or selection oligonucleotide synthesis on a solid support, e.g., in an array format have been described, for example, in WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752 and Zhou et al., Nucleic Acids Res. 32: 5409-5417 (2004).

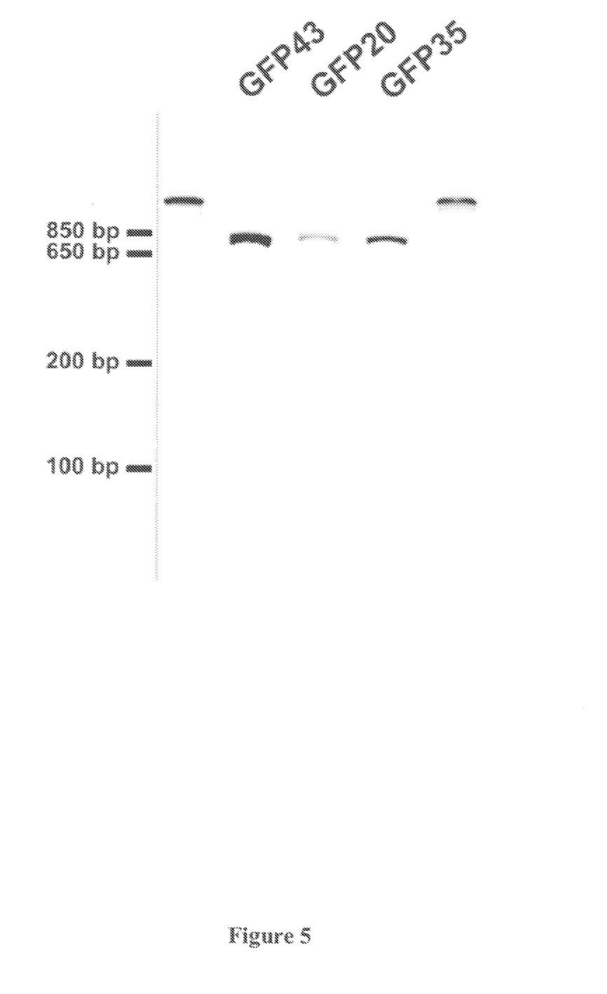

In an exemplary embodiment, construction and/or selection oligonucleotides may be synthesized on a solid support using maskless array synthesizer (MAS). Maskless array synthesizers are described, for example, in PCT application No. WO 99/42813 and in corresponding U.S. Pat. No. 6,375,903. Other examples are known of maskless instruments which can fabricate a custom DNA microarray in which each of the features in the array has a single stranded DNA molecule of desired sequence (See FIG. 5 of U.S. Pat. No. 6,375,903, based on the use of reflective optics). It is often desirable that a maskless array synthesizer is under software control. Since the entire process of microarray synthesis can be accomplished in only a few hours, and since suitable software permits the desired DNA sequences to be altered at will, this class of device makes it possible to fabricate microarrays including DNA segments of different sequences every day or even multiple times per day on one instrument. The differences in DNA sequence of the DNA segments in the microarray can also be slight or dramatic, it makes no different to the process. The MAS instrument may be used in the form it would normally be used to make microarrays for hybridization experiments, but it may also be adapted to have features specifically adapted for the compositions, methods, and systems described herein. For example, it may be desirable to substitute a coherent light source, i.e. a laser, for the light source shown in FIG. 5 of the above-mentioned U.S. Pat. No. 6,375,903. If a laser is used as the light source, a beam expanded and scatter plate may be used after the laser to transform the narrow light beam from the laser into a broader light source to illuminate the micromirror arrays used in the maskless array synthesizer. It is also envisioned that changes may be made to the flow cell in which the microarray is synthesized. In particular, it is envisioned that the flow cell can be compartmentalized, with linear rows of array elements being in fluid communication with each other by a common fluid channel, but each channel being separated from adjacent channels associated with neighboring rows of array elements. During microarray synthesis, the channels all receive the same fluids at the same time. After the DNA segments are separated from the substrate, the channels serve to permit the DNA segments from the row of array elements to congregate with each other and begin to self-assemble by hybridization.

Other methods synthesizing construction and/or selection oligonucleotides include, for example, light-directed methods utilizing masks, flow channel methods, spotting methods, pin-based methods, and methods utilizing multiple supports.

Light directed methods utilizing masks (e.g., VLSIPS™ methods) for the synthesis of oligonucleotides is described, for example, in U.S. Pat. Nos. 5,143,854, 5,510,270 and 5,527,681. These methods involve activating predefined regions of a solid support and then contacting the support with a preselected monomer solution. Selected regions can be activated by irradiation with a light source through a mask much in the manner of photolithography techniques used in integrated circuit fabrication. Other regions of the support remain inactive because illumination is blocked by the mask and they remain chemically protected. Thus, a light pattern defines which regions of the support react with a given monomer. By repeatedly activating different sets of predefined regions and contacting different monomer solutions with the support, a diverse array of polymers is produced on the support. Other steps, such as washing unreacted monomer solution from the support, can be used as necessary. Other applicable methods include mechanical techniques such as those described in U.S. Pat. No. 5,384,261.

Additional methods applicable to synthesis of construction and/or selection oligonucleotides on a single support are described, for example, in U.S. Pat. No. 5,384,261. For example reagents may be delivered to the support by either (1) flowing within a channel defined on predefined regions or (2) “spotting” on predefined regions. Other approaches, as well as combinations of spotting and flowing, may be employed as well. In each instance, certain activated regions of the support are mechanically separated from other regions when the monomer solutions are delivered to the various reaction sites.

Flow channel methods involve, for example, microfluidic systems to control synthesis of oligonucleotides on a solid support. For example, diverse polymer sequences may be synthesized at selected regions of a solid support by forming flow channels on a surface of the support through which appropriate reagents flow or in which appropriate reagents are placed. One of skill in the art will recognize that there are alternative methods of forming channels or otherwise protecting a portion of the surface of the support. For example, a protective coating such as a hydrophilic or hydrophobic coating (depending upon the nature of the solvent) is utilized over portions of the support to be protected, sometimes in combination with materials that facilitate wetting by the reactant solution in other regions. In this manner, the flowing solutions are further prevented from passing outside of their designated flow paths.

Spotting methods for preparation of oligonucleotides on a solid support involve delivering reactants in relatively small quantities by directly depositing them in selected regions. In some steps, the entire support surface can be sprayed or otherwise coated with a solution, if it is more efficient to do so. Precisely measured aliquots of monomer solutions may be deposited dropwise by a dispenser that moves from region to region. Typical dispensers include a micropipette to deliver the monomer solution to the support and a robotic system to control the position of the micropipette with respect to the support, or an ink jet printer. In other embodiments, the dispenser includes a series of tubes, a manifold, an array of pipettes, or the like so that various reagents can be delivered to the reaction regions simultaneously.

Pin-based methods for synthesis of oligonucleotide sequences on a solid support are described, for example, in U.S. Pat. No. 5,288,514. Pin-based methods utilize a support having a plurality of pins or other extensions. The pins are each inserted simultaneously into individual reagent containers in a tray. An array of 96 pins is commonly utilized with a 96-container tray, such as a 96-well microtitre dish. Each tray is filled with a particular reagent for coupling in a particular chemical reaction on an individual pin. Accordingly, the trays will often contain different reagents. Since the chemical reactions have been optimized such that each of the reactions can be performed under a relatively similar set of reaction conditions, it becomes possible to conduct multiple chemical coupling steps simultaneously.

In yet another embodiment, a plurality of oligonucleotide sequences may be synthesized on multiple supports. One example is a bead based synthesis method which is described, for example, in U.S. Pat. Nos. 5,770,358, 5,639,603, and 5,541,061. For the synthesis of molecules such as oligonucleotides on beads, a large plurality of beads are suspended in a suitable carrier (such as water) in a container. The beads are provided with optional spacer molecules having an active site to which is complexed, optionally, a protecting group. At each step of the synthesis, the beads are divided for coupling into a plurality of containers. After the nascent oligonucleotide chains are deprotected, a different monomer solution is added to each container, so that on all beads in a given container, the same nucleotide addition reaction occurs. The beads are then washed of excess reagents, pooled in a single container, mixed and re-distributed into another plurality of containers in preparation for the next round of synthesis. It should be noted that by virtue of the large number of beads utilized at the outset, there will similarly be a large number of beads randomly dispersed in the container, each having a unique oligonucleotide sequence synthesized on a surface thereof after numerous rounds of randomized addition of bases. An individual bead may be tagged with a sequence which is unique to the double-stranded oligonucleotide thereon, to allow for identification during use.

Various exemplary protecting groups useful for synthesis of oligonucleotide sequences on a solid support are described in, for example, Atherton et al., 1989, Solid Phase Peptide Synthesis, IRL Press.

In various embodiments, the methods described herein utilize solid supports for immobilization of oligonucleotide sequences. For example, oligonucleotide sequences may be synthesized on one or more solid supports. Exemplary solid supports include, for example, slides, beads, chips, particles, strands, gels, sheets, tubing, spheres, containers, capillaries, pads, slices, films, or plates. In various embodiments, the solid supports may be biological, non-biological, organic, inorganic, or combinations thereof. When using supports that are substantially planar, the support may be physically separated into regions, for example, with trenches, grooves, wells, or chemical barriers (e.g., hydrophobic coatings, etc.). Supports that are transparent to light are useful when the assay involves optical detection (see e.g., U.S. Pat. No. 5,545,531). The surface of the solid support will typically contain reactive groups, such as carboxyl, amino, and hydroxyl or may be coated with functionalized silicon compounds (see e.g., U.S. Pat. No. 5,919,523).

In certain exemplary embodiments, the oligonucleotide sequences synthesized on the solid support may be used as a template for the production of oligonucleotides for assembly into longer polynucleotide constructs (e.g., nucleic acid sequences of interest). For example, the support-bound oligonucleotides may be contacted with primers that hybridize to the oligonucleotides under conditions that permit chain extension of the primers. The support bound duplexes may then be denatured and subjected to further rounds of amplification.

In other exemplary embodiments, the support bound oligonucleotide sequences may be removed from the solid support prior to amplification and/or assembly into polynucleotide constructs (e.g., nucleic acid sequences of interest). The oligonucleotides may be removed from the solid support, for example, by exposure to conditions such as acid, base, oxidation, reduction, heat, light, metal ion catalysis, displacement or elimination chemistry, or by enzymatic cleavage.

In certain embodiments, oligonucleotide sequences may be attached to a solid support through a cleavable linkage moiety. For example, the solid support may be functionalized to provide cleavable linkers for covalent attachment to the oligonucleotides. The linker moiety may be of six or more atoms in length. Alternatively, the cleavable moiety may be within an oligonucleotide and may be introduced during in situ synthesis. A broad variety of cleavable moieties are available in the art of solid phase and microarray oligonucleotide synthesis (see e.g., Pon, R., Methods Mol. Biol. 20:465-496 (1993); Verma et al., Ann. Rev. Biochem. 67:99-134 (1998); U.S. Pat. Nos. 5,739,386, 5,700,642 and 5,830,655; and U.S. Patent Publication Nos. 2003/0186226 and 2004/0106728). A suitable cleavable moiety may be selected to be compatible with the nature of the protecting group of the nucleoside bases, the choice of solid support, and/or the mode of reagent delivery, among others. In an exemplary embodiment, the oligonucleotides cleaved from the solid support contain a free 3′-OH end. Alternatively, the free 3′-OH end may also be obtained by chemical or enzymatic treatment, following the cleavage of oligonucleotides. The cleavable moiety may be removed under conditions which do not degrade the oligonucleotides. Preferably the linker may be cleaved using two approaches, either (a) simultaneously under the same conditions as the deprotection step or (b) subsequently utilizing a different condition or reagent for linker cleavage after the completion of the deprotection step.

The covalent immobilization site may either be at the 5′ end of the oligonucleotide or at the 3′ end of the oligonucleotide. In some instances, the immobilization site may be within the oligonucleotide (i.e. at a site other than the 5′ or 3′ end of the oligonucleotide). The cleavable site may be located along the oligonucleotide backbone, for example, a modified 3′-5′ internucleotide linkage in place of one of the phosphodiester groups, such as ribose, dialkoxysilane, phosphorothioate, and phosphoramidate internucleotide linkage. The cleavable oligonucleotide analogs may also include a substituent on, or replacement of, one of the bases or sugars, such as 7-deazaguanosine, 5-methylcytosine, inosine, uridine, and the like.

In one embodiment, cleavable sites contained within the modified oligonucleotide may include chemically cleavable groups, such as dialkoxysilane, 3′-(S)-phosphorothioate, 5′-(S)-phosphorothioate, 3′-(N)-phosphoramidate, 5′-(N)phosphoramidate, and ribose. Synthesis and cleavage conditions of chemically cleavable oligonucleotides are described in U.S. Pat. Nos. 5,700,642 and 5,830,655. For example, depending upon the choice of cleavable site to be introduced, either a functionalized nucleoside or a modified nucleoside dimer may be first prepared, and then selectively introduced into a growing oligonucleotide fragment during the course of oligonucleotide synthesis. Selective cleavage of the dialkoxysilane may be effected by treatment with fluoride ion. Phosphorothioate internucleotide linkage may be selectively cleaved under mild oxidative conditions. Selective cleavage of the phosphoramidate bond may be carried out under mild acid conditions, such as 80% acetic acid. Selective cleavage of ribose may be carried out by treatment with dilute ammonium hydroxide.

In another embodiment, a non-cleavable hydroxyl linker may be converted into a cleavable linker by coupling a special phosphoramidite to the hydroxyl group prior to the phosphoramidite or H-phosphonate oligonucleotide synthesis as described in U.S. Patent Application Publication No. 2003/0186226. The cleavage of the chemical phosphorylation agent at the completion of the oligonucleotide synthesis yields an oligonucleotide bearing a phosphate group at the 3′ end. The 3′-phosphate end may be converted to a 3′ hydroxyl end by a treatment with a chemical or an enzyme, such as alkaline phosphatase, which is routinely carried out by those skilled in the art.

In another embodiment, the cleavable linking moiety may be a TOPS (two oligonucleotides per synthesis) linker (see e.g., PCT publication WO 93/20092). For example, the TOPS phosphoramidite may be used to convert a non-cleavable hydroxyl group on the solid support to a cleavable linker. A preferred embodiment of TOPS reagents is the Universal TOPS™ phosphoramidite. Conditions for Universal TOPS™ phosphoramidite preparation, coupling and cleavage are detailed, for example, in Hardy et al. Nucleic Acids Research 22(15):2998-3004 (1994). The Universal TOPS™ phosphoramidite yields a cyclic 3′ phosphate that may be removed under basic conditions, such as the extended ammonia and/or ammonia/methylamine treatment, resulting in the natural 3′ hydroxy oligonucleotide.

In another embodiment, a cleavable linking moiety may be an amino linker. The resulting oligonucleotides bound to the linker via a phosphoramidite linkage may be cleaved with 80% acetic acid yielding a 3′-phosphorylated oligonucleotide.

In another embodiment, the cleavable linking moiety may be a photocleavable linker, such as an ortho-nitrobenzyl photocleavable linker. Synthesis and cleavage conditions of photolabile oligonucleotides on solid supports are described, for example, in Venkatesan et al., J. Org. Chem. 61:525-529 (1996), Kahl et al., J. Org. Chem. 64:507-510 (1999), Kahl et al., J. Org. Chem. 63:4870-4871 (1998), Greenberg et al., J. Org. Chem. 59:746-753 (1994), Holmes et al., J. Org. Chem. 62:2370-2380 (1997), and U.S. Pat. No. 5,739,386. Ortho-nitrobenzyl-based linkers, such as hydroxymethyl, hydroxyethyl, and Fmoc-aminoethyl carboxylic acid linkers, may also be obtained commercially.

In another embodiment, oligonucleotides may be removed from a solid support by an enzyme such as a nuclease. For example, oligonucleotides may be removed from a solid support upon exposure to one or more restriction endonucleases, including, for example, class IIs restriction enzymes. A restriction endonuclease recognition sequence may be incorporated into the immobilized oligonucleotides and the oligonucleotides may be contacted with one or more restriction endonucleases to remove the oligonucleotides from the support. In various embodiments, when using enzymatic cleavage to remove the oligonucleotides from the support, it may be desirable to contact the single stranded immobilized oligonucleotides with primers, polymerase and dNTPs to form immobilized duplexes. The duplexes may then be contacted with the enzyme (e.g., a restriction endonuclease) to remove the duplexes from the surface of the support. Methods for synthesizing a second strand on a support bound oligonucleotide and methods for enzymatic removal of support bound duplexes are described, for example, in U.S. Pat. No. 6,326,489. Alternatively, short oligonucleotides that are complementary to the restriction endonuclease recognition and/or cleavage site (e.g., but are not complementary to the entire support bound oligonucleotide) may be added to the support bound oligonucleotides under hybridization conditions to facilitate cleavage by a restriction endonuclease (see e.g., PCT Publication No. WO 04/024886).

In various embodiments, the methods disclosed herein comprise amplification of nucleic acids including, for example, oligonucleotides, subassemblies and/or polynucleotide constructs (e.g., nucleic acid sequences of interest). Amplification may be carried out at one or more stages during an assembly scheme and/or may be carried out one or more times at a given stage during assembly. Amplification methods may comprise contacting a nucleic acid with one or more primers that specifically hybridize to the nucleic acid under conditions that facilitate hybridization and chain extension. Exemplary methods for amplifying nucleic acids include the polymerase chain reaction (PCR) (see, e.g., Mullis et al. (1986) Cold Spring Harb. Symp. Quant. Biol. 51 Pt 1:263 and Cleary et al. (2004) Nature Methods 1:241; and U.S. Pat. Nos. 4,683,195 and 4,683,202), anchor PCR, RACE PCR, ligation chain reaction (LCR) (see, e.g., Landegran et al. (1988) Science 241:1077-1080; and Nakazawa et al. (1994) Proc. Natl. Acad. Sci. U.S.A. 91:360-364), self sustained sequence replication (Guatelli et al. (1990) Proc. Nall. Acad. Sci. U.S.A. 87:1874), transcriptional amplification system (Kwoh et al. (1989) Proc. Natl. Acad. Sci. U.S.A. 86:1173), Q-Beta Replicase (Lizardi et al. (1988) BioTechnology 6:1197), recursive PCR (Jaffe et al. (2000) J. Biol. Chem. 275:2619; and Williams et al. (2002) J. Biol. Chem. 277:7790), the amplification methods described in U.S. Pat. Nos. 6,391,544, 6,365,375, 6,294,323, 6,261,797, 6,124,090 and 5,612,199, or any other nucleic acid amplification method using techniques well known to those of skill in the art. In exemplary embodiments, the methods disclosed herein utilize PCR amplification.

In certain exemplary embodiments, methods for amplifying nucleic acid sequences are provided. Exemplary methods for amplifying nucleic acids include the polymerase chain reaction (PCR) (see, e.g., Mullis et al. (1986) Cold Spring Harb. Symp. Quant. Biol. 51 Pt 1:263 and Cleary et al. (2004) Nature Methods 1:241; and U.S. Pat. Nos. 4,683,195 and 4,683,202), anchor PCR, RACE PCR, ligation chain reaction (LCR) (see, e.g., Landegran et al. (1988) Science 241:1077-1080; and Nakazawa et al. (1994) Proc. Natl. Acad. Sci. U.S.A. 91:360-364), self sustained sequence replication (Guatelli et al. (1990) Proc. Natl. Acad. Sci. U.S.A. 87:1874), transcriptional amplification system (Kwoh et al. (1989) Proc. Natl. Acad. Sci. U.S.A. 86:1173), Q-Beta Replicase (Lizardi et al. (1988) BioTechnology 6:1197), recursive PCR (Jaffe et al. (2000) J. Biol. Chem. 275:2619; and Williams et al. (2002) J. Biol. Chem. 277:7790), the amplification methods described in U.S. Pat. Nos. 6,391,544, 6,365,375, 6,294,323, 6,261,797, 6,124,090 and 5,612,199, isothermal amplification (e.g., rolling circle amplification (RCA), hyperbranched rolling circle amplification (HRCA), strand displacement amplification (SDA), helicase-dependent amplification (HDA), PWGA) or any other nucleic acid amplification method using techniques well known to those of skill in the art.

“Polymerase chain reaction,” or “PCR,” refers to a reaction for the in vitro amplification of specific DNA sequences by the simultaneous primer extension of complementary strands of DNA. In other words, PCR is a reaction for making multiple copies or replicates of a target nucleic acid flanked by primer binding sites, such reaction comprising one or more repetitions of the following steps: (i) denaturing the target nucleic acid, (ii) annealing primers to the primer binding sites, and (iii) extending the primers by a nucleic acid polymerase in the presence of nucleoside triphosphates. Usually, the reaction is cycled through different temperatures optimized for each step in a thermal cycler instrument. Particular temperatures, durations at each step, and rates of change between steps depend on many factors well-known to those of ordinary skill in the art, e.g., exemplified by the references: McPherson et al., editors, PCR: A Practical Approach and PCR2: A Practical Approach (IRL Press, Oxford, 1991 and 1995, respectively). For example, in a conventional PCR using Taq DNA polymerase, a double stranded target nucleic acid may be denatured at a temperature greater than 90° C., primers annealed at a temperature in the range 50-75° C., and primers extended at a temperature in the range 72-78° C.

The term “PCR” encompasses derivative forms of the reaction, including but not limited to, RT-PCR, real-time PCR, nested PCR, quantitative PCR, multiplexed PCR, assembly PCR and the like. Reaction volumes range from a few hundred nanoliters, e.g., 200 mL, to a few hundred microliters, e.g., 200 microliters. “Reverse transcription PCR,” or “RT-PCR,” means a PCR that is preceded by a reverse transcription reaction that converts a target RNA to a complementary single stranded DNA, which is then amplified, e.g., Tecott et al., U.S. Pat. No. 5,168,038. “Real-time PCR” means a PCR for which the amount of reaction product, i.e., amplicon, is monitored as the reaction proceeds. There are many forms of real-time PCR that differ mainly in the detection chemistries used for monitoring the reaction product, e.g., Gelfand et al., U.S. Pat. No. 5,210,015 (“Taqman”); Wittwer et al., U.S. Pat. Nos. 6,174,670 and 6,569,627 (intercalating dyes); Tyagi et al., U.S. Pat. No. 5,925,517 (molecular beacons). Detection chemistries for real-time PCR are reviewed in Mackay et al., Nucleic Acids Research, 30:1292-1305 (2002). “Nested PCR” means a two-stage PCR wherein the amplicon of a first PCR becomes the sample for a second PCR using a new set of primers, at least one of which binds to an interior location of the first amplicon. As used herein, “initial primers” in reference to a nested amplification reaction mean the primers used to generate a first amplicon, and “secondary primers” mean the one or more primers used to generate a second, or nested, amplicon. “Multiplexed PCR” means a PCR wherein multiple target sequences (or a single target sequence and one or more reference sequences) are simultaneously carried out in the same reaction mixture, e.g. Bernard et al. (1999) Anal. Biochem., 273:221-228 (two-color real-time PCR). Usually, distinct sets of primers are employed for each sequence being amplified. “Quantitative PCR” means a PCR designed to measure the abundance of one or more specific target sequences in a sample or specimen. Techniques for quantitative PCR are well-known to those of ordinary skill in the art, as exemplified in the following references: Freeman et al., Biotechniques, 26:112-126 (1999); Becker-Andre et al., Nucleic Acids Research, 17:9437-9447 (1989); Zimmerman et al., Biotechniques, 21:268-279 (1996); Diviacco et al., Gene, 122:3013-3020 (1992); Becker-Andre et al., Nucleic Acids Research, 17:9437-9446 (1989); and the like.

In certain embodiments, methods of determining the sequence of one or more nucleic acid sequences of interest are provided. Determination of the sequence of a nucleic acid sequence of interest can be performed using variety of sequencing methods known in the art including, but not limited to, sequencing by hybridization (SBH), sequencing by ligation (SBL), quantitative incremental fluorescent nucleotide addition sequencing (QIFNAS), stepwise ligation and cleavage, fluorescence resonance energy transfer (FRET), molecular beacons, TaqMan reporter probe digestion, pyrosequencing, fluorescent in situ sequencing (FISSEQ), FISSEQ beads (U.S. Pat. No. 7,425,431), wobble sequencing (PCT/US05/27695), multiplex sequencing (U.S. Ser. No. 12/027,039, filed Feb. 6, 2008; Porreca et al (2007) Nat. Methods 4:931), polymerized colony (POLONY) sequencing (U.S. Pat. Nos. 6,432,360, 6,485,944 and 6,511,803, and PCT/US05/06425); nanogrid rolling circle sequencing (ROLONY) (U.S. Ser. No. 12/120,541, filed May 14, 2008), allele-specific oligo ligation assays (e.g., oligo ligation assay (OLA), single template molecule OLA using a ligated linear probe and a rolling circle amplification (RCA) readout, ligated padlock probes, and/or single template molecule OLA using a ligated circular padlock probe and a rolling circle amplification (RCA) readout) and the like. High-throughput sequencing methods, e.g., on cyclic array sequencing using platforms such as Roche 454, Illumina Solexa, AB-SOLiD, Helicos, Polonator platforms and the like, can also be utilized. High-throughput sequencing methods are described in U.S. Ser. No. 61/162,913, filed Mar. 24, 2009. A variety of light-based sequencing technologies are known in the art (Landegren et al. (1998) Genome Res. 8:769-76; Kwok (2000) Pharmocogenomics 1:95-100; and Shi (2001) Clin. Chem. 47:164-172).

It is to be understood that the embodiments of the present invention which have been described are merely illustrative of some of the applications of the principles of the present invention. Numerous modifications may be made by those skilled in the art based upon the teachings presented herein without departing from the true spirit and scope of the invention. The contents of all references, patents and published patent applications cited throughout this application are hereby incorporated by reference in their entirety for all purposes.

The following examples are set forth as being representative of the present invention. These examples are not to be construed as limiting the scope of the invention as these and other equivalent embodiments will be apparent in view of the present disclosure, figures tables and accompanying claims.

### Example I

### Scalable Gene Synthesis Platform Using High-Fidelity DNA Microchips

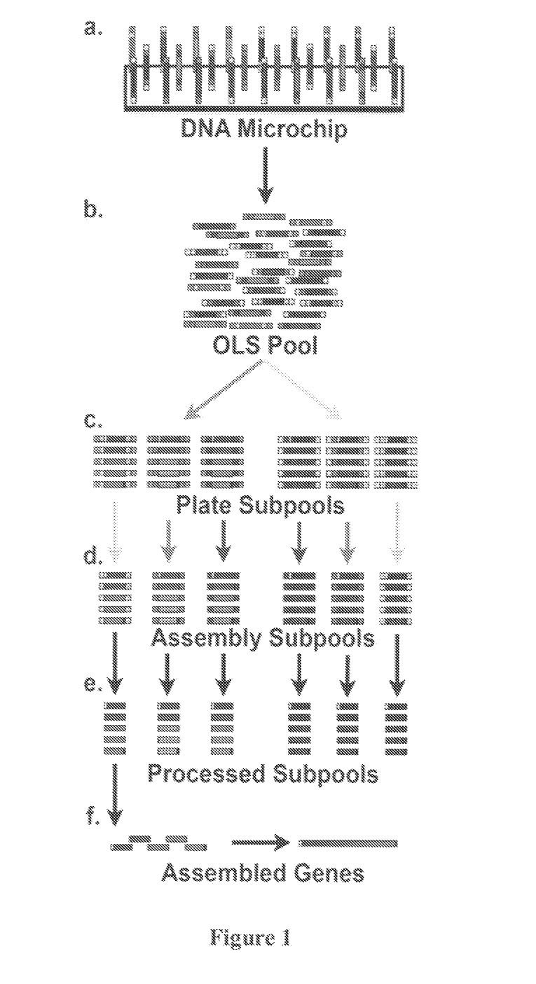

Oligonucleotide Library Synthesis (OLS) pools were used as a starting point for more scalable DNA microchip-based gene synthesis methods. Two OLS pools (OLS Pools 1 and 2) of different lengths were designed, each containing approximately 13,000 130mer or 200mer oligonucleotides, respectively. FIG. 1 depicts a general schematic of the methods described herein for utilizing OLS pools in a gene synthesis platform. Briefly, oligonucleotides were designed that were then printed on DNA microchips, which were then recovered as a mixed pool of oligonucleotides (OLS Pool). Next, the long oligonucleotide lengths were taken advantage of to independently amplify and process only those oligonucleotides required for a given gene assembly. For the 200mer OLS Pool 2, this was a two step process where first a “plate subpool” was amplified that contained DNA to construct up to 96 genes, and then individual “assembly subpools” were amplified to separate the oligonucleotides for each particular assembly. For the 130mer OLS Pool 1, direct amplification into assembly subpools was performed, foregoing the plate subpool step. Next, the primers used for the amplification steps were removed by either Type IIS restriction endonucleases to form double-stranded DNA (dsDNA) fragments (OLS Pool 2), or a combination of enzymatic steps to form single-stranded DNA (ssDNA) fragments (OLS Pool 1). Finally, PCR assembly was used to construct full-length genes, perform enzymatic error correction to improve error rates if necessary, and finally clone and characterize the constructs.

TABLE 1

| | Pre-PCR OLS | Post-PCR OLS |
|---|---|---|
| 55K SLXA | Pool | Pool |
| Total Reads | 757126 | 830659 |
| Mapped reads | 530616 | 616713 |
| Mapped reads <34 bp | 14426 | 20982 |
| Imperfect Oligos | 67050 | 78769 |
| Avg Error of Imperfect | 1.67 | 1.69 |
| Oligo | | |
| Phred30 Imperfect Oligos | 28812 | 29033 |
| Phred30 Average Error of | 1.286 | 1.305 |
| Imperfect Oligo | | |
| Matches | 18466976 | 21454745 |
| Transitions | 24569 | 56377 |
| Transversions | 66905 | 81820 |
| Deletions | 19761 | 24016 |
| Insertions | 839 | 935 |
| Match % | 99.40% | 99.25% |
| Transition % | 0.13% | 0.26% |
| Transversion % | 0.36% | 0.38% |
| Deletion % | 0.11% | 0.11% |
| Insertion % | 0.00% | 0.00% |
| Phred30 Matches | 17443050 | 20217413 |
| Phred30 Transitions | 10914 | 8908 |
| Phred30 Transversions | 10743 | 10369 |
| Phred30 Deletions | 14795 | 17965 |
| Phred30 Insertions | 600 | 659 |
| Phred30 Match % | 99.79% | 99.81% |
| Phred30 Transition % | 0.06% | 0.04% |
| Phred30 Transversion % | 0.06% | 0.05% |
| Phred30 Deletion % | 0.08% | 0.09% |
| Phred30 Insertion % | 0.00% | 0.00% |

Table 1 depicts data from reanalysis of Agilent OLS libraries for quantitation of error rates (Li et al. (2009) Genome Research 19:1606). The dataset was realigned using Exonerate to allow for gapped alignments and analysis of indels (Slater et al. (2005) BMC Bioinformatics 6:31). Specifically, an affine local alignment model was used that is equivalent to the classic Smith-Waterman-Gotoh alignment, a gap extension of −5, and used the full refine option to allow for dynamic programming based optimization of the alignment. The alignments were then mapped, and quality scores were converted to Phred values using the alignments and the Maq utility sol2sanger (Li. Maq: Mapping and Assembly with Qualities. Wellcome Trust Sanger Institute. 2010). Sequences were then analyzed to determine error rates using custom python scripts that analyzed the types of errors and could filter the statistics based on quality scores. While this method provided an estimate for error rates, without intending to be bound by scientific theory, unmapped reads are likely to have higher error rates, and quality scores in next-generation sequencing are not directly comparable to expected Sanger error rates.

Obtaining subpools of only those DNA fragments required for any particular assembly was important for robust gene synthesis in very large DNA backgrounds. To facilitate this, 20mer PCR primer sets with low potential cross-hybridization (“orthogonal” primers) were designed (Xu, Q. et al. Design of 240,000 orthogonal 25mer DNA barcode probes. Proc. Natl. Acad. Sci. USA 106, 2289-2294 (2009)). Two separate orthogonal primer sets were constructed for the different OLS pools because of their varying requirements for downstream processing. Both sets were screened for potential cross-hybridization, low secondary structure, and matched melting temperatures to construct large sets of orthogonal PCR primer pairs.

To construct genes from the OLS pools, automated algorithms were developed to split the sequence into overlapping segments with matching melting temperatures such that they could be later assembled by PCR. Genes on OLS Pool 1 and 2 were designed differently to test the effect of different overlap lengths. Genes on OLS Pool 1 were designed such that the processed ssDNA pools fully overlapped to form a complete dsDNA sequence. In OLS Pool 2, the processed dsDNA fragments partially overlapped by approximately 20 bp and could be assembled into a contiguous gene sequence using PCR. A set of fluorescent proteins was initially constructed to test the efficacy of the gene synthesis methods on both OLS Pools.

For OLS Pool 1, two independent “assembly subpools” were designed that encoded for GFPmut3b plus flanking orthogonal primer sequences that were later used for PCR assembly (“construction primers”). The two assembly subpools, GFP43 and GFP35, differed in the average overlap length (43 bp and 35 bp, respectively), total length (82-90 and 64-78 bases, respectively), and number of oligonucleotides (18 and 22, respectively). Two subpools (Control Subpools 1 & 2) containing ten and five 130mer oligonucleotides, respectively, were also designed to test amplification efficacy. The other eight subpools, containing a total of 12,945 130mer sequences, were constructed on the same chip but were not used in this study except to provide potential sources of cross-hybridization. Each of these 12 subpools was flanked with independent orthogonal primer pairs (“assembly-specific primers”). As a control, these same algorithms were used to design a set of shorter CPG oligonucleotides (20 bp average overlap) encoding GFPmut3b (obtained from IDT). These oligonucleotides were combined to form a third pool that was also tested (“GFP20”).

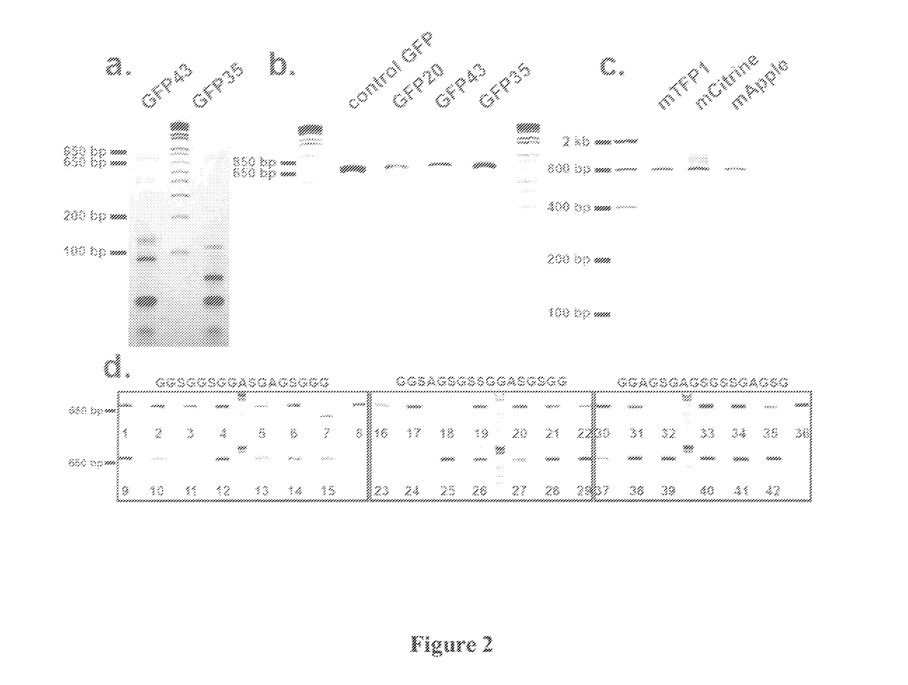

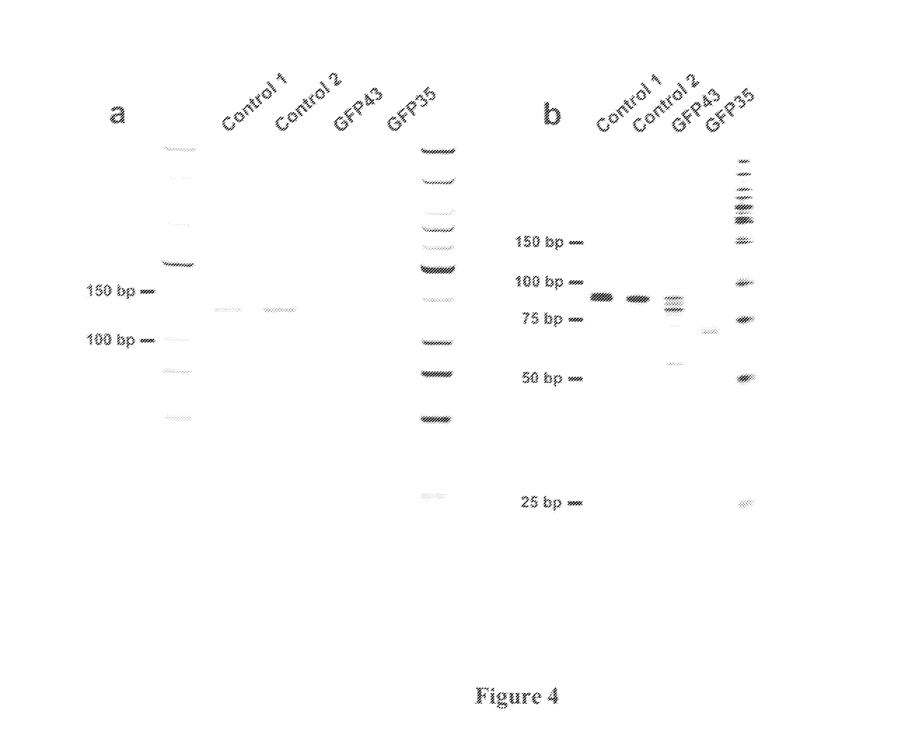

Each of the four subpools (GFP43, GFP35, Control 1, and Control 2) were PCR amplified from the synthesized OLS pool using modified primers that facilitated downstream processing. Since the GFP43 and GFP35 subpools had different oligonucleotide lengths than the rest of OLS Pool 1, the size difference displayed in the GFP43 and GFP35 subpools compared to the Control 1 and 2 subpools indicated that no detectable oligonucleotides from other subpools were present (see FIG. 4A). The oligonucleotides were then processed to remove primer sequences (see FIG. 4B). Briefly, lambda exonuclease was used to make the PCR products single stranded, and then uracil DNA glycosylase, Endonuclease VIII, and DpnII restriction endonuclease were used to cleave off the assembly-specific primers. The resultant gel indicated that while the reaction was efficient, unprocessed oligonucleotide still remained. In addition, spurious cleavage by DpnII was observed which, without intending to be bound by scientific theory, was likely due to the extensive overlap within the subpool that is inherent in the gene synthesis process. The GFP43, GFP35, and GFP20 subpools were assembled using PCR, which resulted in GFP-sized products as well as many incorrect low molecular weight products (FIG. 2A). The presence of the full-length products indicated that the all the designed oligonucleotides were present in both subpools.

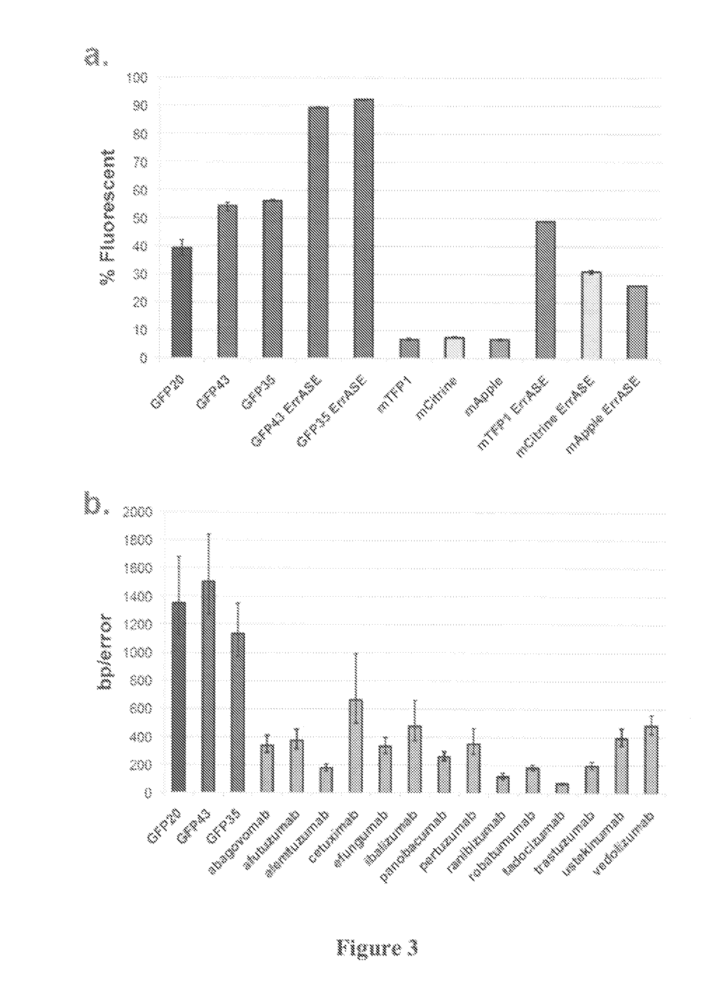

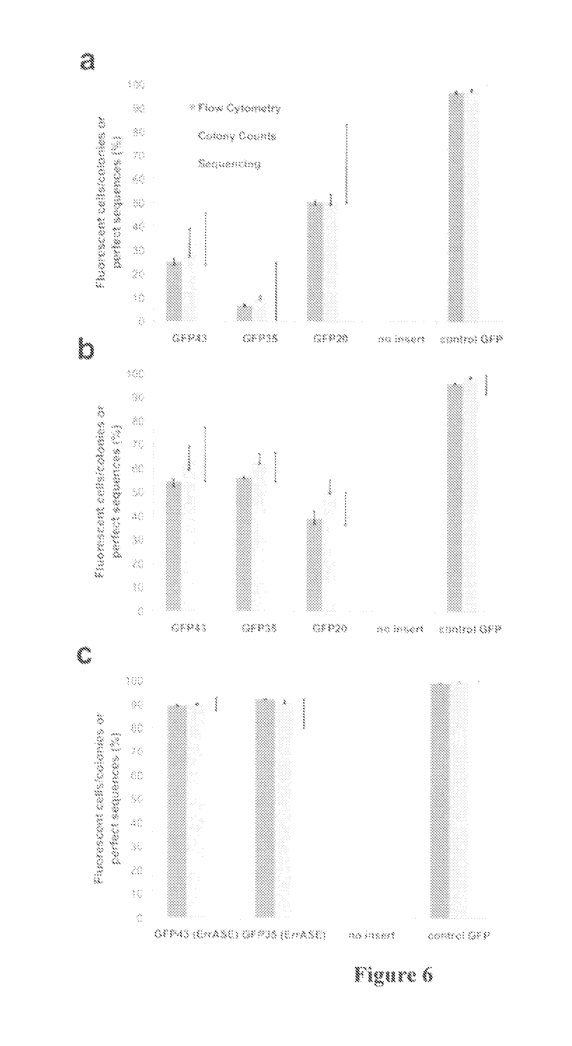

The assembly products were gel isolated, re-amplified by PCR, digested, and then cloned into an expression vector. After re-amplification, secondary bands appeared, which upon sequencing displayed a large number of short, misassembled products in the GFP35 assembly (see FIG. 5). The above procedure was repeated, omitting the re-amplification step, which eliminated the short misassemblies (FIG. 2B). Flow cytometry tests, manual colony counts, and sequencing of individual clones were used to measure the error rates (see FIG. 6). All three of the assays correlated well, and the error rates determined through sequencing were 1/1,500 bp, 1/1130 bp, and 1/1,350 bp for the GFP43, GFP35, and GFP20 synthesis reactions, respectively (See FIG. 3 and Table 2).

TABLE 2

| | | | | | | | Large | Large | | | | |
|---|---|---|---|---|---|---|---|---|---|---|---|---|
| | Good | | | Sequenced | Mis- | Small | Deletions | Deletion | | Bp/ | Poisson | Poisson |
| Construct | Reads | Missassemblies | Perfect | Bases | matches | Deletions | (>2 bp) | Size | Insertions | Error | High | Low |
| GFP20 | 49 | 4 | 28 | 35133 | 0 | 3 | 0 | 0 | 6 | 1351 | 330 | 222 |
| GFP43 | 63 | 1 | 44 | 45171 | 5 | 17 | 0 | 0 | 8 | 1506 | 336 | 232 |
| GFP43 (ErrASE) | 30 | 0 | 27 | 21510 | 3 | 0 | 0 | 0 | 0 | 7170 | 9794 | 2624 |
| GFP35 | 60 | 0 | 36 | 43020 | 5 | 29 | 0 | 0 | 4 | 1132 | 219 | 158 |
| GFP35 (ErrASE) | 28 | 0 | 24 | 20076 | 1 | 3 | 0 | 0 | 0 | 5019 | 5019 | 1673 |
| abagovomab | 15 | 0 | 1 | 11175 | 20 | 12 | 0 | 0 | 1 | 339 | 71 | 50 |
| afutuzumab | 15 | 1 | 2 | 11580 | 24 | 7 | 0 | 0 | 0 | 374 | 82 | 57 |
| alemtuzumab | 12 | 0 | 0 | 8913 | 22 | 19 | 9 | 99 | 0 | 178 | 29 | 22 |
| cetuximab | 8 | 0 | 2 | 5960 | 6 | 3 | 0 | 0 | 0 | 662 | 331 | 166 |
| efungumab | 16 | 0 | 2 | 11945 | 27 | 8 | 1 | 23 | 0 | 332 | 66 | 47 |
| ibalizumab | 8 | 0 | 0 | 6224 | 11 | 2 | 0 | 0 | 0 | 479 | 184 | 104 |
| panobacumab | 22 | 1 | 3 | 16707 | 38 | 23 | 3 | 13 | 0 | 261 | 37 | 29 |
| pertuzumab | 8 | 0 | 3 | 5959 | 10 | 4 | 2 | 25 | 1 | 351 | 112 | 68 |
| ranibizumab | 4 | 2 | 0 | 2948 | 7 | 11 | 7 | 80 | 0 | 118 | 29 | 20 |
| robatumumab | 21 | 0 | 0 | 14860 | 36 | 20 | 24 | 911 | 2 | 181 | 22 | 18 |
| tadocizumab | 7 | 8 | 0 | 5200 | 43 | 18 | 1 | 15 | 13 | 69 | 9 | 7 |
| trastuzumab | 16 | 0 | 1 | 11772 | 24 | 25 | 10 | 196 | 1 | 196 | 29 | 22 |
| ustekinumab | 23 | 0 | 6 | 17336 | 32 | 11 | 1 | 6 | 0 | 394 | 70 | 52 |
| vedolizumab | 33 | 0 | 6 | 25571 | 43 | 9 | 1 | 4 | 0 | 482 | 77 | 58 |

Table 2 depicts the sequencing results obtained for cloned assemblies. The results from sequencing 11 constructs generated from IDT oligonucleotides (GFP20), OLS Pool 1 (GFP43 and GFP35), and OLS Pool 2 (antibodies). “Good Read” refers to the number of clones that returned sequence information (there were no bad reads). “Misassemblies” refer to sequences that did not have the complete sequence cloned and usually came from sequences of less than 200 bp. “Perfect Reads” refers to the number of clones that had sequence exactly equivalent to the designed sequence. “Sequenced Bases” refer to the total number of sequenced bases homologous to the designed sequence, and “Mismatches” refer to the number of mismatches from the designed sequence. “Small Indels” and “Large Indels” refer to the number of deletions <3 or >2 bp long, respectively. “Lg Del Size” refers to the sum of deletions present in all reads in the large indels. “Insertions” refer to the number of inserted bases in the sequence compared to the reference. The “Bp/Error” refers to the average error rate, and in this case, considers each large indel to be a single “error.” “Poisson High” and “Poisson Low” are the expected Poisson noise (minus and plus the square of the number of errors, respectively).

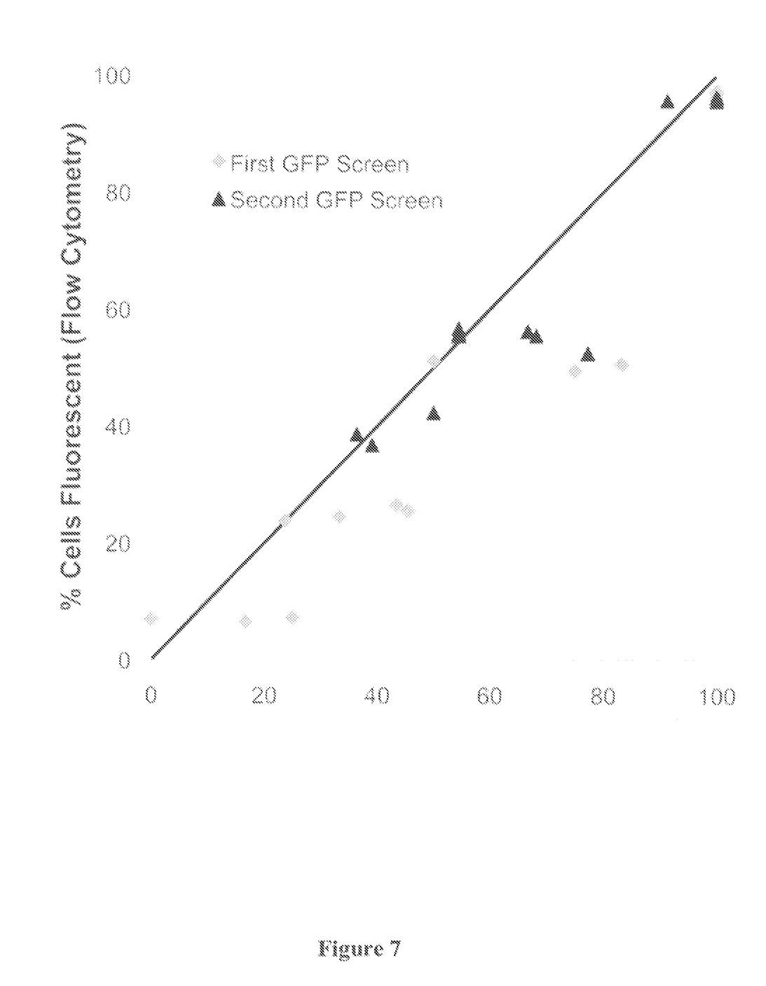

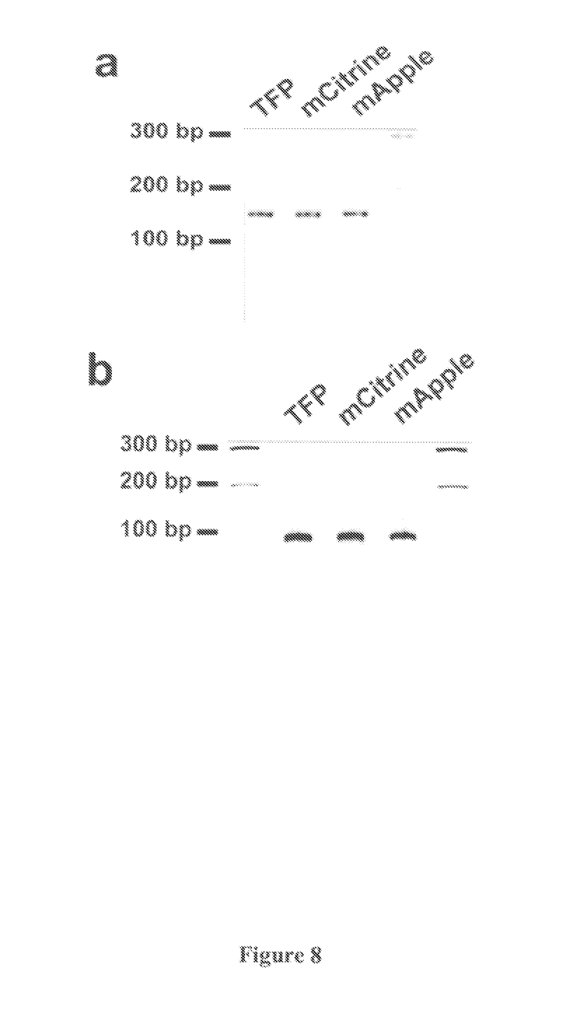

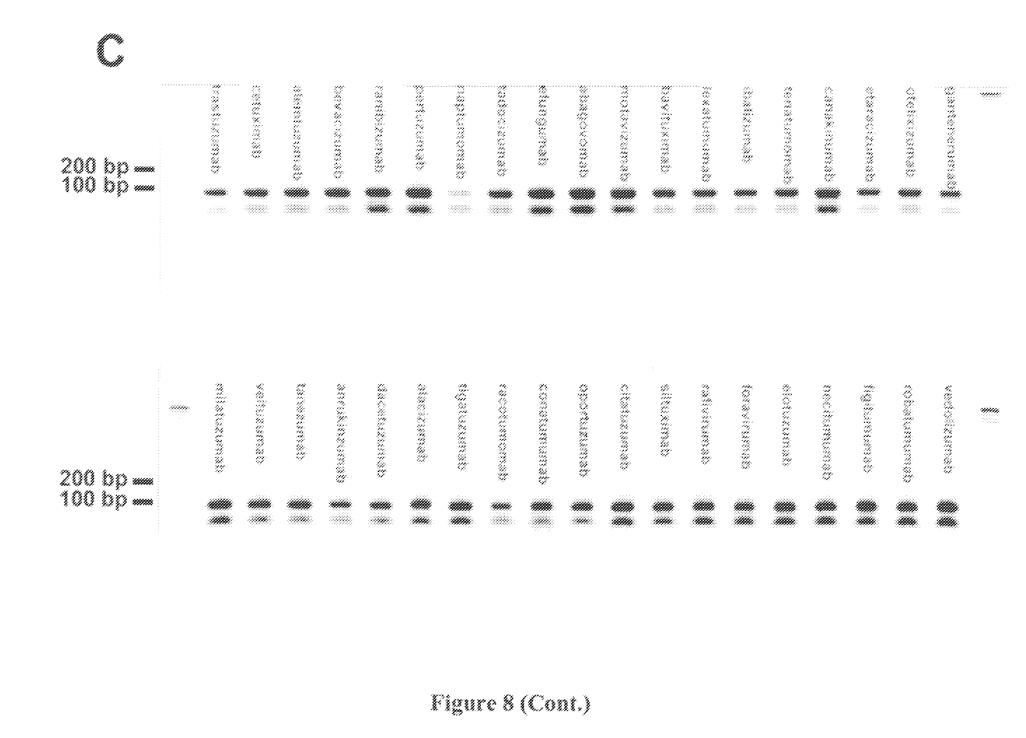

Without intending to be bound by scientific theory, these results demonstrated a number of important results. First, the subpool assembly primers were sufficiently well-designed to provide stringent subpool amplification of as few as five oligonucleotides out of a 12,995 oligonucleotide background. Second, the relative quantities of the oligonucleotides in the assembly subpools were sufficient to allow PCR assembly. Third, the error rates from 130mer OLS pools were sufficient to construct gene-sized fragments (717 bp) such that >50% of the sequences would be perfect. In fact, the error rates from both the GFP43 and GFP35 assemblies were indistinguishable from the column-synthesized GFP20 assemblies. Finally, these data indicate that the level of fluorescence of the gene assemblies correlated with the number of constructs with perfect sequence, providing a useful screen to test fluorescent gene assemblies in OLS Pool 2 (see FIG. 7).

In OLS Pool 2, 836 assembly subpools were designed and split into 11 plate subpools, encoding 2,456,706 bases of oligonucleotides that could potentially result in 869,125 bp of final assembled sequence. Three fluorescent proteins were constructed to test assembly protocols in OLS Pool 2: mTFP1, mCitrine, and mApple. The PCR assembly protocols developed for ssDNA subpools in OLS Pool 1 only produced short (less than 200 bp) misassemblies when applied the dsDNA subpools in OLS Pool 2. By screening over 1,000 assembly PCR conditions, it was determined that three factors affected the robust assembly of full-length products. A pre-assembly step of 15-20 thermal cycles performed in the absence of construction primers was performed followed by a shortened 20-30 cycles of assembly PCR with the construction primer. Second, low annealing temperatures (50-55° C.) were used during the pre-assembly and more stringent annealing temperatures were used during the assembly PCR (60-72° C.). Finally, the amount of DNA added to the pre-assembly was two to three orders of magnitude greater than the assemblies in OLS Pool 1. Using these optimized protocols, the three genes were assembled with no detectable misassemblies, thereby removing the need for gel isolation (FIG. 2C). Cloning followed by flow cytometry screening showed that 6.8%, 7.5%, and 6.8% of the cells were fluorescent for mTFP1, mCitrine, and mApple assemblies, respectively (see FIG. 3A).

Assuming 6% correct sequence per construct and no selection against errors in the assembly process, the error rate was approximately 1/250 bp for 200mer OLS Pool 2. This error rate is significantly above that of the estimates for 130mer OLS Pool 1 (approximately 1/1000 bp) and the sequenced 55K 150mer OLS pool (approximately 1/500 bp). Despite the higher error rate, there were several advantages to the 200mer OLS Pool 2. First, the extensive overlaps designed in OLS Pool 1 caused spurious processing of the primers from the assembly subpools. The use of Type IIs restriction endonucleases to process primers to form dsDNA resulted in more robust processing. Second, while the 13,000 features in OLS Pool 1 can be used to construct greater than 700 genes, each subpool amplification used 1/500^{th} of the total chip-eluted DNA. While it maybe possible to run this process with 1/1000^{th} the total material, there was a concern that the use of larger OLS Pools would be difficult (e.g., a 55,000 feature OLS pool would require 1/3,000^{th} of the total material). The longer 200mers of OLS Pool 2 allowed for a first plate amplification before the assembly amplification, which facilitated process scaling to larger OLS Pools. Third, the assemblies of OLS Pool 1 produced many smaller bands and required lower-throughput gel isolation procedures. Without intending to be bound by scientific theory, this could be due to mispriming during PCR assembly because of the long overlap lengths used in the design process. The assemblies in OLS Pool 2 used much shorter overlap lengths, and resulted in no smaller molecular weight misassembled products.

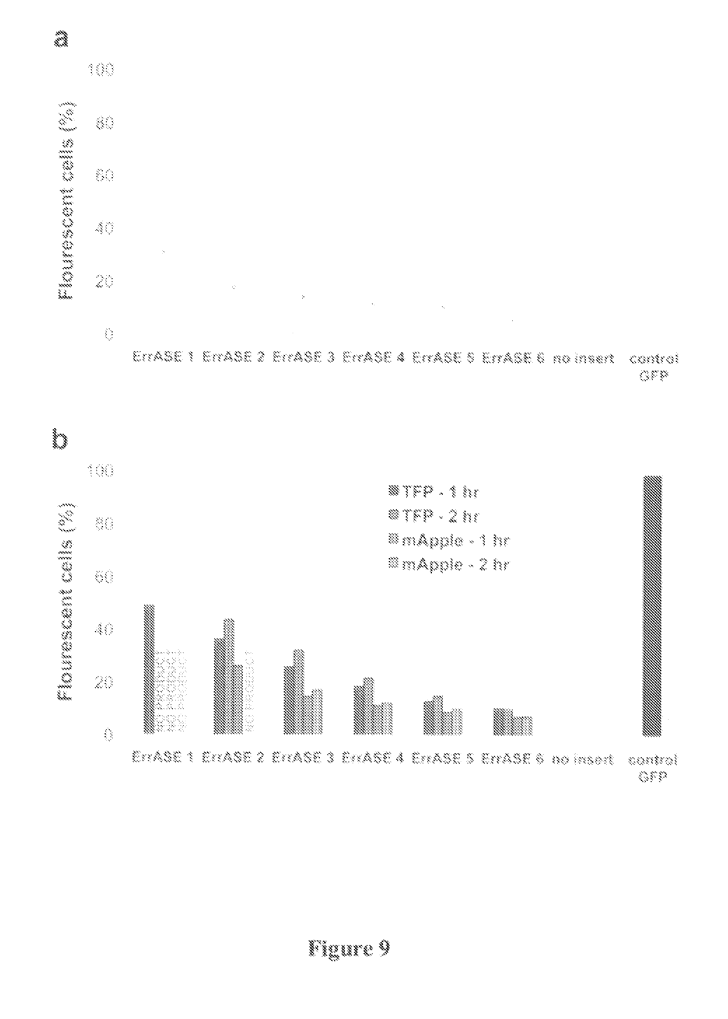

In order to improve the error rates of the genes assembled from OLS Pool 2, ErrASE, a commercially-available enzyme cocktail, was used to remove errors in the assembled fluorescent proteins. Briefly, assembled genes are denatured and re-annealed to allow for the formation of hetero-duplexes. A resolvase enzyme in ErrASE then recognizes and cuts at mismatched positions. Other enzymes in the cocktail remove these cut mismatched positions. The products could then be reamplified by PCR to reassemble the full-length gene. For each gene, ErrASE was applied at six different stringencies, the constructs were re-amplified, PCR products were cloned, and the cloned genes were re-screened using flow cytometry. Improvement of the level of fluorescence progressively increased with increased ErrASE stringency. At the highest levels of error correction, the fluorescence levels were 31%, 49%, and 26% for mTFP1, mCitrine, and mApple respectively (see FIGS. 3A and 9). The ErrASE procedure was also performed on the GFP43 and GFP35 pools from OLS Pool 1, resulting in fluorescence levels of 89% and 92% respectively (see FIGS. 3A and 9). Clones of GFP43 and GFP35 were sequenced, and 3 errors in 21,510 ( 1/7170 bp) and 4 errors in 20,076 ( 1/5019 bp) sequenced bases were identified, respectively.

As a more challenging test for the DNA synthesis technology described herein, oligonucleotides were designed and synthesized for 42 genes encoding single-chain Fv (scFv) regions corresponding to a number of well-known antibodies in OLS Pool 2. Certain genes have been difficult to synthesize using commercial gene synthesis companies. Without intending to be bound by scientific theory, this might be partly due to the prototype (Gly₄Ser)₃ linker, which is designed to maximize flexibility and allow the heavy and light V regions to assemble (Huston, J. S. et al. Medical applications of single-chain antibodies. Int. Rev Immunol. 10, 195-217 (1993)). The repetitive nature and high GC content of the linker-encoding sequences often represents a challenge for accurate DNA synthesis. Three different linker sequences were tested: GGSGGSGGASGAGSGGG (Linker 1) (SEQ ID NO:1), GGSAGSGSSGGASGSGG (Linker 2) (SEQ ID NO:2), and GAGSGAGSGSSGAGSG (Linker 3) (SEQ ID NO:3), that varied in GC content and repetitive character of the linker encoding sequence. In addition, the presence of high sequence homology in the antibody backbones and linkers represented a potential source of cross-hybridization that could interfere with assembly.

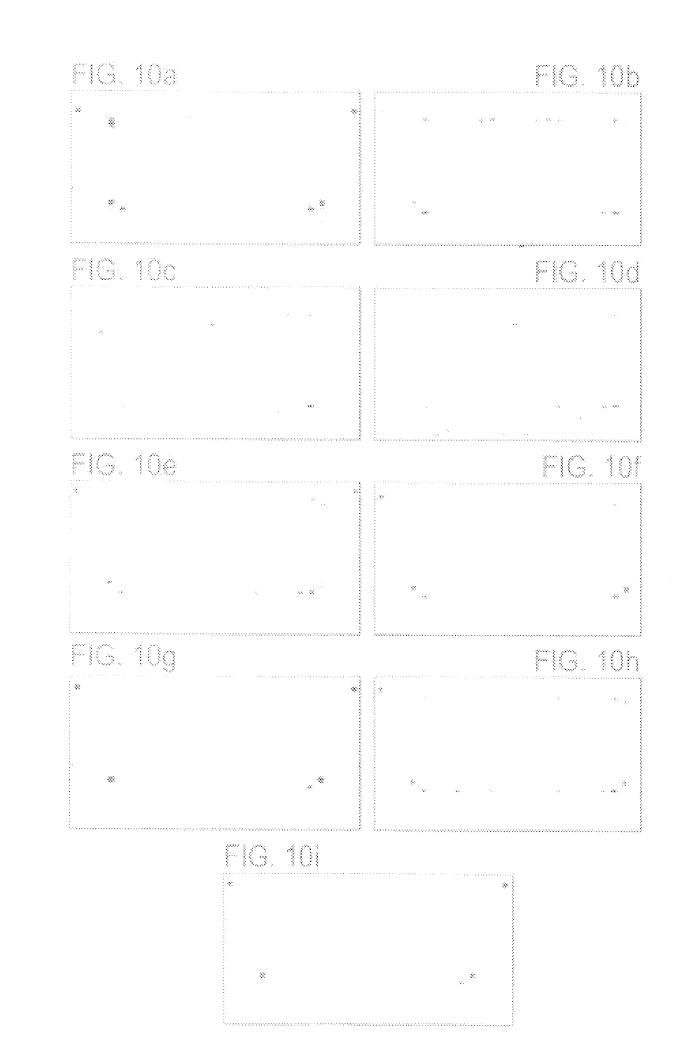

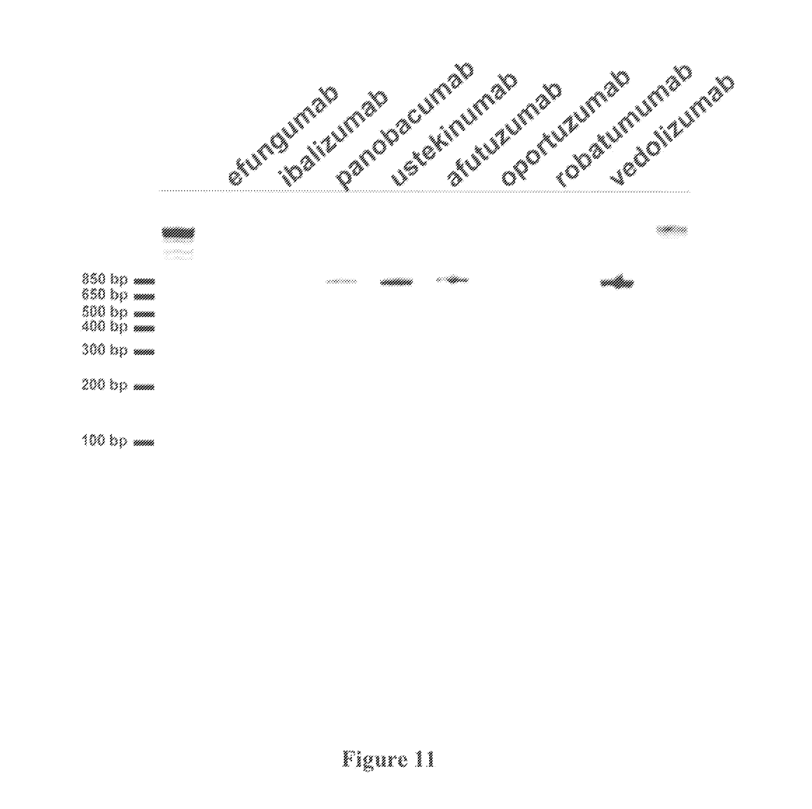

As expected, the antibody sequences did not assemble as robustly as the fluorescent proteins and, thus, conditions during pre- and post-assembly were further optimized (see FIG. 10). Using one protocol, 40 of the 42 constructs assembled to the correct size (see FIG. 2D and Table 3). The two misassembled genes displayed faint bands at the correct size, which were gel isolated and reamplified to produce strong bands of the correct size. 15 antibodies were chosen for expression (5 with Linker 1, 4 with Linker 2, and 6 with Linker 3). Enzymatic error correction was performed using ErrASE. The product was gel isolated and the constructs were cloned into an expression vector (See FIG. 11). One of the 15 antibodies did not clone, and another had a deleted linker region in all 21 sequenced clones. Both of these antibodies were encoded with the highest GC content linker. The average error rate of the 14 antibodies that did clone was 1/315 bp (see FIG. 3B and Table 2); this was significantly higher than the GFP assemblies, but still sufficient for construction of genes of this size (approximately 10% of clones should be perfect on average). In addition, sequence analysis showed no instances of subpool cross-contamination during the assembly process.

TABLE 3

| | | Primers | | Band from | Reaction | Perfect Clone |
|---|---|---|---|---|---|---|
| Name | ID # | (subpool/construction) | Linker | Assembly? | Cloned | Found? |
| trastuzumab | 1 | 301/101 | GGSGGSGGASGAGSGGG | yes | 2 | yes |
| bevacizumab | 2 | 304/104 | GGSGGSGGASGAGSGGG | yes | | |
| pertuzumab | 3 | 306/106 | GGSGGSGGASGAGSGGG | yes | 2 | yes |
| efungumab | 4 | 309/109 | GGSGGSGGASGAGSGGG | yes | 1 and 2 | yes |
| bavituximab | 5 | 312/112 | GGSGGSGGASGAGSGGG | yes | | |
| tenatumomab | 6 | 315/115 | GGSGGSGGASGAGSGGG | yes | | |
| otelixizumab | 7 | 318/118 | GGSGGSGGASGAGSGGG | no (very | | |
| | | | | faint) | | |
| gantenerumab | 8 | 320/120 | GGSGGSGGASGAGSGGG | yes | | |
| tanezumab | 9 | 323/123 | GGSGGSGGASGAGSGGG | yes | | |
| dacetuzumab | 10 | 326/126 | GGSGGSGGASGAGSGGG | yes | | |
| racotumomab | 11 | 329/129 | GGSGGSGGASGAGSGGG | yes | | |
| oportuzumab | 12 | 332/132 | GGSGGSGGASGAGSGGG | yes | 1 (none | |
| | | | | | sequenced) | |
| rafivirumab | 13 | 335/135 | GGSGGSGGASGAGSGGG | yes | | |
| elotuzumab | 14 | 338/138 | GGSGGSGGASGAGSGGG | yes | | |
| robatumumab | 15 | 341/141 | GGSGGSGGASGAGSGGG | yes | 1 | no |
| cetuximab | 16 | 302/102 | GGSAGSGSSGGASGSGG | yes | 2 | yes |
| ranibizumab | 17 | 305/105 | GGSAGSGSSGGASGSGG | yes | 2 | no |
| naptumomab | 18 | 307/107 | GGSAGSGSSGGASGSGG | yes | | |
| abagovomab | 19 | 310/110 | GGSAGSGSSGGASGSGG | yes | 2 | yes |
| lexatumumab | 20 | 313/113 | GGSAGSGSSGGASGSGG | yes | | |
| canakinumab | 21 | 316/116 | GGSAGSGSSGGASGSGG | yes | | |
| milatuzumab | 22 | 321/121 | GGSAGSGSSGGASGSGG | yes | | |
| anrukinzumab | 23 | 324/124 | GGSAGSGSSGGASGSGG | yes | | |
| alacizumab | 24 | 327/127 | GGSAGSGSSGGASGSGG | no | | |
| conatumumab | 25 | 330/130 | GGSAGSGSSGGASGSGG | yes | | |
| citatuzumab | 26 | 333/133 | GGSAGSGSSGGASGSGG | yes | | |
| foravirumab | 27 | 336/136 | GGSAGSGSSGGASGSGG | yes | | |
| necitumumab | 28 | 339/139 | GGSAGSGSSGGASGSGG | yes | | |
| vedolizumab | 29 | 342/142 | GGSAGSGSSGGASGSGG | yes | 1 | yes |
| veltuzumab | 30 | 322/122 | GGAGSGAGSGSSGAGSG | yes | | |
| panobacumab | 31 | 319/119 | GGAGSGAGSGSSGAGSG | yes | 1 | yes |
| etaracizumab | 32 | 317/117 | GGAGSGAGSGSSGAGSG | yes | | |
| ibalizumab | 33 | 314/114 | GGAGSGAGSGSSGAGSG | yes | 1 | no |
| motavizumab | 34 | 311/111 | GGAGSGAGSGSSGAGSG | yes | | |
| tadocizumab | 35 | 308/108 | GGAGSGAGSGSSGAGSG | yes | 2 | no |
| alemtuzumab | 36 | 303/103 | GGAGSGAGSGSSGAGSG | yes | 2 | no |
| figitumumab | 37 | 340/140 | GGAGSGAGSGSSGAGSG | yes | | |
| farletuzumab | 38 | 337/137 | GGAGSGAGSGSSGAGSG | yes | | |
| siltuximab | 39 | 334/134 | GGAGSGAGSGSSGAGSG | yes | | |
| afutuzumab | 40 | 331/131 | GGAGSGAGSGSSGAGSG | yes | 1 | yes |
| tigatuzumab | 41 | 328/128 | GGAGSGAGSGSSGAGSG | yes | | |
| ustekinumab | 42 | 325/125 | GGAGSGAGSGSSGAGSG | yes | 1 | yes |

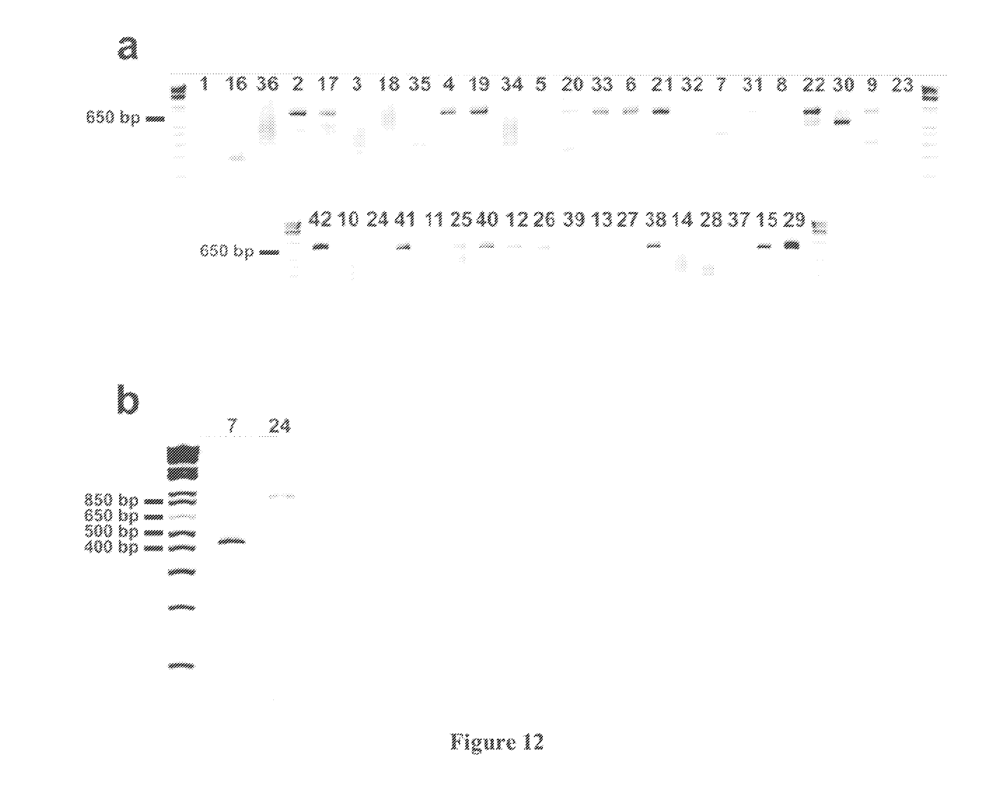

Table 3 depicts assembly results from 42 attempted antibody constructions. Of the 42 assemblies of antibody subpools from OLS Pool 2, 29 of the first set of reactions (FIG. 12A) and 40 of the second set (FIG. 3D) resulted in products of the correct size. An attempt to clone 8 from the first set of assemblies (7 cloned successfully) and 8 from the second (all cloned successfully) was performed. The “ID #” refers to the number used in FIG. 3D to identify the antibody. Primers are the primer numbers set forth below, with a forward and reverse primer pair corresponding to each number (for instance, skpp-301-F and skpp-301-R are the assembly subpool amplification primers for trastuzumab). Linker refers to the amino acid sequence used to link the heavy and the light chain. Band from assembly? refers to presence of a band of the correct size refers to the gel in FIG. 2D. The Reaction cloned column indicates whether the antibody was cloned from either of two assembly reaction (assembly 1 shown in FIG. 11, assembly 2 shown in FIG. 3D). Perfect clone found? indicates whether or not at least one of the cloned assemblies sequenced contained no errors. The sequence identifiers of the sequences set forth in Table 3 are as follows: trastuzumab-BtsI-20 (SEQ ID NO:4), Cetuximab-BtsI-20 (SEQ ID NO:5), alemtuzumab-BtsI-20 (SEQ ID NO:6), bevacizumab-BtsI-20 (SEQ ID NO:7), ranibizumab-BtsI-20 (SEQ ID NO:8), pertuzumab-BtsI-20 (SEQ ID NO:9), naptumomab-BtsI-20 (SEQ ID NO:10), tadocizumab-BtsI-20 (SEQ ID NO:11), efungumab-BtsI-20 (SEQ ID NO:12), Abagovomab-BtsI-20 (SEQ ID NO:13), Motavizumab-BtsI-20 (SEQ ID NO:14), bavituximab-BtsI-20 (SEQ ID NO:15), lexatumumab-BtsI-20 (SEQ ID NO:16), ibalizumab-BtsI-20 (SEQ ID NO:17), tenatumomab-BtsI-20 (SEQ ID NO:18), canakinumab-BtsI-20 (SEQ ID NO:19), etaracizumab-BtsI-20 (SEQ ID NO:20), otelixizumab-BtsI-20 (SEQ ID NO:21), Panobacumab-BtsI-20 (SEQ ID NO:22), gantenerumab-BtsI-20 (SEQ ID NO:23), milatuzumab-BtsI-20 (SEQ ID NO:24), veltuzumab-BtsI-20 (SEQ ID NO:25), Tanezumab-BtsI-20 (SEQ ID NO:26), anrukinzumab-BtsI-20 (SEQ ID NO:27), ustekinumab-BtsI-20 (SEQ ID NO:28), dacetuzumab-BtsI-20 (SEQ ID NO:29), Alacizumab-BtsI-20 (SEQ ID NO:30), tigatuzumab-BtsI-20 (SEQ ID NO:31), Racotumomab-BtsI-20 (SEQ ID NO:32), conatumumab-BtsI-20 (SEQ ID NO:33), afutuzumab-BtsI-20 (SEQ ID NO:34), oportuzumab-BtsI-20 (SEQ ID NO:35), citatuzumab-BtsI-20 (SEQ ID NO:36), siltuximab-BtsI-20 (SEQ ID NO:37), rafivirumab-BtsI-20 (SEQ ID NO:38), Foravirumab-BtsI-20 (SEQ ID NO:39), Farletuzumab-BtsI-20 (SEQ ID NO:40), Elotuzumab-BtsI-20 (SEQ ID NO:41), necitumumab-BtsI-20 (SEQ ID NO:42), figitumumab-BtsI-20 (SEQ ID NO:43), Robatumumab-BtsI-20 (SEQ ID NO:44), and vedolizumab-BtsI-20 (SEQ ID NO:45).

The results presented herein demonstrate for the first time the assembly of gene-sized DNA fragments totaling approximately 25,000 bp from oligonucleotide pools of more than 50 kilobases. Two separate OLS pool sizes and assembly methods are described, each of which has their own advantages and disadvantages. The shorter, 130mer OLS Pool 1 assemblies had lower error rates, but because there are no plate amplifications, will be harder to scale when larger OLS pools are utilized. The longer 200mer OLS Pool 2 was easier to scale, but contained higher error rates. The costs of oligonucleotides in both processes are less than $0.01/bp of final synthesized sequence, and thus the dominant costs become enzymatic processing, cloning, and sequence verification. The final cost of such a process will depend upon the application. If one can select for functional constructs, the longer OLS pools would provide the lowest costs and highest scales. However, if perfect sequence is required, sequencing 12-24 clones would add $0.05-$0.10/bp to the cost. Thus, the use of shorter OLS pools would be ideal. Future work on lowering cost of perfect sequence will focus on both the ability to lower sequencing costs such as by using cheaper next-generation sequencing technologies, or by incorporating other error-correction techniques such as PAGE selection of oligonucleotide pools or mutS-based error filtration (Tian (2004) (supra); Carr, P. A. et al. Protein-mediated error correction for de novo DNA synthesis. Nucleic Acids Res. 32, e162 (2004)).

TABLE 4
OLS Pool 1 Primer Sequences

| Name | Forward | Reverse |
|---|---|---|
| GFP43 | AACACGTCCGTCCTAGA | GCAAGCGGTACACTCAGATC |
| | ACT (SEQ ID NO: 46) | (SEQ ID NO: 50) |
| GFP35 | AGTGTTGAGCGTAACCA | CAGGAGTTGTCTAGGCGATC |
| | AGT (SEQ ID NO: 47) | (SEQ ID NO: 51) |
| Control 1 | AAGCAAGATTCTCGTCG | TGTAAGGCACATCTCGGATC |
| | GAT (SEQ ID NO: 48) | (SEQ ID NO: 51) |
| Control 2 | TCTAATCTAGCGCGACG | CCACAAGAGGCGCTATGATC |
| | TCT (SEQ ID NO: 49) | (SEQ ID NO: 53) |

Table 4 sets forth OLS Pool 1 subpool amplification primers.

TABLE 5

| | |
|---|---|
| GFPmut3_43_0,1-for | AACACGTCCGTCCTAGAACTGATA |
| | GGGTGACTGCTTTCGCGTACAGGT |
| | ACCATGAGTAAAGGAGAAGAA |
| | CTTTTCACTGGAGTTGTCCCAAT |
| | TCTTGTTGAAGATCTGAGTGTAC |
| | CGCTTGC (SEQ ID NO: 54) |
| GFPmut3_43_2,3-for | AACACGTCCGTCCTAGAACTTTAGA |
| | TGGTGATGTTAATGGGCACAAA |
| | TTTTCTGTCAGTGGAGAGG |
| | GTGAAGGTGATGCAACATACG |
| | GAAAACTTACCCTTAAATTTAG |
| | ATCTGAGTGTACCGCTTGC |
| | (SEQ ID NO: 55) |
| GFPmut3_43_4,5-for | AACACGTCCGTCCTAGAACTTTTGC |
| | ACTACTGGAAAACTACCTGTT |
| | CCATGGCCAACACTTGTCA |
| | CTACTTTCGGTTATGGTGTTC |
| | AATGCTTTGCGAGATAGATCT |
| | GAGTGTACCGCTTGC |
| | (SEQ ID NO: 56) |
| GFPmut3_43_6,7-for | AACACGTCCGTCCTAGAACTCCCAG |
| | ATCATATGAAACAGCATGAC |
| | TTTTTCAAGAGTGCCATGCC |
| | CGAAGGTTATGTACAGGAAA |
| | GAACTATATTTTTCAAAGGAT |
| | CTGAGTGTACCGCTTGC |
| | (SEQ ID NO: 57) |
| GFPmut3_43_8,9-for | AACACGTCCGTCCTAGAACTATGA |
| | CGGGAACTACAAGACACGTG |
| | CTGAAGTCAAGTTTGAAG |
| | GTGATACCCTTGTTAATAGAAT |
| | CGAGTTAAAAGGTATTGATTTT |
| | GATCTGAGTGTACCGCTTGC |
| | (SEQ ID NO: 58) |
| GFPmut3_43_10,11-for | AACACGTCCGTCCTAGAACTAAAGA |
| | AGATGGAACATTCTTGGACACAAATTGGA |
| | ATACAACTATAACTCACACAATGTATA |
| | CATCATGGCAGACAAACAAA |
| | AGAATGGAGATCTGAGTGTACCGCTTGC |
| | (SEQ ID NO: 59) |
| GFPmut3_43_12,13- | AACACGTCCGTCCTAGAACTATCAAA |
| for | GTTAACTTCAAAATTAGACACAAC |
| | ATTGAAGATGGAAGCGTT |
| | CAACTAGCAGACCATTATCAAC |
| | AAAATACTCCAATTGGCGATGAT |
| | CTGAGTGTACCGCTTGC |
| | (SEQ ID NO: 60) |
| GFPmut3_43_14,15- | AACACGTCCGTCCTAGAACTGGCCCT |
| for | GTCCTTTTACCAGACAACCATTA |
| | CCTGTCCACACAATCTGCCCT |
| | TTCGAAAGATCCCAACGAAAAGA |
| | GAGACCACATGGTCCGATCTG |
| | AGTGTACCGCTTGC |
| | (SEQ ID NO: 61) |
| GFPmut3_43_16,17- | AACACGTCCGTCCTAGAACTTTCTTG |
| for | AGTTTGTAACAGCTGCTGGGATTA |
| | CACATGGCATGGATGAACTATACAA |
| | ATAAAAGCTTACTTCTTCTCGGTCG |
| | CATGAGGCTGGATCTGAGTGTACC |
| | GCTTGC (SEQ ID NO: 62) |
| GFPmut3_43_1,2-rev | AACACGTCCGTCCTAGAACTCTCCA |
| | CTGACAGAAAATTTGTGCCCATTAA |
| | CATCACCATCTAATTCAACAAGAAT |
| | TGGGACAACTCCAGTGAAAAGTTCT |
| | TCTCGATCTGAGTGTACCGCTTGC |
| | (SEQ ID NO: 63) |
| GFPmut3_43_3,4-rev | AACACGTCCGTCCTAGAACTAAGTGT |
| | TGGCCATGGAACAGGTAGTTTTCC |
| | AGTAGTGCAAATAAATTTAAGGGTA |
| | AGTTTTCCGTATGTTGCATCACCT |
| | TCACCCTGATCTGAGTGTACCGCTTGC |
| | (SEQ ID NO: 64) |
| GFPmut3_43_5,6-rev | AACACGTCCGTCCTAGAACTATGG |
| | CACTCTTGAAAAAGTCATGCTGTTT |
| | CATATGATCTGGGTATCTCGCAAAG |
| | CATTGAACACCATAACCGA |
| | AAGTAGTGACGATCTGAGTGTACCG |
| | CTTGC (SEQ ID NO: 65) |
| GFPmut3_43_7,8-rev | AACACGTCCGTCCTAGAACTTTCA |
| | AACTTGACTTCAGCACGTGTCTTGTA |
| | GTTCCCGTCATCTTTGAAAAATATAGT |
| | TCTTTCCTGTACATAACCTTCGGGCGA |
| | TCTGAGTGTACCGCTTGC |
| | (SEQ ID NO: 66) |
| GFPmut3_43_9,10- | AACACGTCCGTCCTAGAACTAT |
| rev | AGTTGTATTCCAATTTGTGTCCAAG |
| | AATGTTTCCATCTTCTTTAAAATCAAT |
| | ACCTTTTAACTCGATTCTATTAACAA |
| | GGGTATCACCGATCTGAG |
| | TGTACCGCTTGC (SEQ ID NO: 67) |
| GFPmut3_43_11,12- | AACACGTCCGTCCTAGAACTG |
| rev | CTTCCATCTTCAATGTTGTGTCT |
| | AATTTTGAAGTTAACTTTGATTCCA |
| | TTCTTTTGTTTGTCTGCCATGATGT |
| | ATACATTGTGTGAGTTGATCTGA |
| | GTGTACCGCTTGC (SEQ ID NO: 68) |
| GFPmut3_43_13,14- | AACACGTCCGTCCTAGAACTA |
| rev | GATTGTGTGGACAGGTAATGG |
| | TTGTCTGGTAAAAGGACAGGGCC |
| | ATCGCCAATTGGAGTATTTTGTTG |
| | ATAATGGTCTGCTAGTTGAACGA |
| | TCTGAGTGTACCGCTTGC |
| | (SEQ ID NO: 69) |
| GFPmut3_43_15,16- | AACACGTCCGTCCTAGAACTCA |
| rev | TCCATGCCATGTGTAATCCCA |
| | GCAGCTGTTACAAACTCAAGAAG |
| | GACCATGTGGTCTCTCTTTTCGTT |
| | GGGATCTTTCGAAAGGGCGATCT |
| | GAGTGTA |
| | CCGCTTGC (SEQ ID NO: 70) |
| GFPmut3_43_10,17- | AACACGTCCGTCCTAGAACTCTT |
| rev-bridge | TACTCATGGTACCTGTACGCG |
| | AAAGCAGTCACCCTATCCAGCCTCATG |
| | CGACCGAGAAGAAGTAAGCTTTTATTTG |
| | TATAGTTGATCTGAGTGTA |
| | CCGCTTGC (SEQ ID NO: 71) |

Table 5 sets forth OLS Pool 1 oligonucleotide sequences for GFP43.

TABLE 6

| | |
|---|---|
| GFPmut3_35_0,1-for | AGTGTTGAGCGTAACCAAGT |
| | GATAGGGTGACTGCTTTCGC |
| | GTACAGGTACCATGAGTAAA |
| | GGAGAAGAACTTTTCACTGGA |
| | GTTGTCCGATCGCCTAGACAA |
| | CTCCTG (SEQ ID NO: 72) |
| GFPmut3_35_2,3-for | AGTGTTGAGCGTAACCAAGTC |
| | AATTCTTGTTGAATTAGATGGT |
| | GATGTTAATGGGCACAAATTTT |
| | CTGTCAGTGGAGAGGGTGAAG |
| | GTGATGATCGCCTAGACAACTC |
| | CTG (SEQ ID NO: 73) |
| GFPmut3_35_4,5-for | AGTGTTGAGCGTAACCAAGTG |
| | CAACATACGGAAAACTTACCC |
| | TTAAATTTATTTGCACTACTGG |
| | AAAACTACCTGTTCCATGGCCA |
| | ACACGATCGCCTAGACAACTC |
| | CTG (SEQ ID NO: 74) |
| GFPmut3_35_6,7-for | AGTGTTGAGCGTAACCAAGTT |
| | TGTCACTACTTTCGGTTATGGT |
| | GTTCAATGCTTTGCGAGATAC |
| | CCAGATCATATGAAACAGCAT |
| | GACGATCGCCTAGACAACTC |
| | CTG (SEQ ID NO: 75) |
| GFPmut3_35_8,9-for | AGTGTTGAGCGTAACCAAGTT |
| | TTTTCAAGAGTGCCATGCCCG |
| | AAGGTTATGTACAGGAAAGAA |
| | CTATATTTTTCAAAGATGACGG |
| | GAAGATCGCCTAGACAACTCC |
| | TG (SEQ ID NO: 76) |
| GFPmut3_35_10,11-for | AGTGTTGAGCGTAACCAAGTCT |
| | ACAAGACACGTGCTGAAGTCAA |
| | GTTTGAAGGTGATACCCTTGTT |
| | AATAGAATCGAGTTAAAAGGTA |
| | TGATCGCCTAGACAACTCCTG |
| | (SEQ ID NO: 77) |
| GFPmut3_35_12,13-for | AGTGTTGAGCGTAACCAAGTT |
| | GATTTTAAAGAAGATGGAAAC |
| | ATTCTTGGACACAAATTGGAA |
| | TACAACTATAACTCACACAAT |
| | GTATACATCATGGGATCGCCT |
| | AGACAACTCCTG (SEQ ID NO: 78) |
| GFPmut3_35_14,15-for | AGTGTTGAGCGTAACCAAGTC |
| | AGACAAACAAAAGAATGGAAT |
| | CAAAGTTAACTTCAAAATTAGA |
| | CACAACATTGAAGATGGAAGC |
| | GTTCAACTGATCGCCTAGACA |
| | ACTCCTG (SEQ ID NO: 79) |
| GFPmut3_35_16,17-for | AGTGTTGAGCGTAACCAAGTA |
| | GCAGACCATTATCAACAAAAT |
| | ACTCCAATTGGCGATGGCCCT |
| | GTCCTTTTACCAGACAACCAT |
| | TACCTGGATCGCCTAGACAAC |
| | TCCTG (SEQ ID NO: 80) |
| GFPmut3_35_18,19-for | AGTGTTGAGCGTAACCAAGT |
| | TCCACACAATCTGCCCTTTC |
| | GAAAGATCCCAACGAAAAGA |
| | GAGACCACATGGTCCTTCTT |
| | GAGTTTGTAACGATCGCCTA |
| | GACAACTCCTG (SEQ ID NO: 81) |
| GFPmut3_35_20,21-for | AGTGTTGAGCGTAACCAAGT |
| | AGCTGCTGGGATTACACATG |
| | GCATGGATGAACTATACAAA |
| | TAAAAGCTTACTTCTTCTCG |
| | GTCGCATGAGGCTGGATCG |
| | CCTAGACAACTCCTG (SEQ ID |
| | NO: 82) |
| GFPmut3_35_1,2-rev | AGTGTTGAGCGTAACCAAGT |
| | TGTGCCCATTAACATCACCA |
| | TCTAATTCAACAAGAATTGG |
| | GACAACTCCAGTGAAAAGTT |
| | CTTCTCCTTTACTCATGATC |
| | GCCTAGACAACTCCTG (SEQ ID |
| | NO: 83) |
| GFPmut3_35_3,4-rev | AGTGTTGAGCGTAACCAAG |
| | TAGTGCAAATAAATTTAAG |
| | GGTAAGTTTTCCGTATGTT |
| | GCATCACCTTCACCCTCTC |
| | CACTGACAGAAAATTGATC |
| | GCCTAGACAACTCCTG (SEQ ID |
| | NO: 84) |
| GFPmut3_35_5,6-rev | AGTGTTGAGCGTAACCAAG |
| | TAAAGCATTGAACACCATA |
| | ACCGAAAGTAGTGACAAG |
| | TGTTGGCCATGGAACAGG |
| | TAGTTTTCCAGTGATCGC |
| | CTAGACAACTCCTG (SEQ ID |
| | NO: 85) |
| GFPmut3_35_7,8-rev | AGTGTTGAGCGTAACCAA |
| | GTCATAACCTTCGGGCAT |
| | GGCACTCTTGAAAAAGTC |
| | ATGCTGTTTCATATGATC |
| | TGGGTATCTCGCGATCG |
| | CCTAGACAACTCCTG (SEQ ID |
| | NO: 86) |
| GFPmut3_35_9,10-rev | AGTGTTGAGCGTAACCAA |
| | GTTTCAAACTTGACTTCAG |
| | CACGTGTCTTGTAGTTCC |
| | CGTCATCTTTGAAAAATA |
| | TAGTTCTTTCCTGTAGAT |
| | CGCCTAGACAACTCCTG (SEQ |
| | ID NO: 87) |
| GFPmut3_35_11,12-rev | AGTGTTGAGCGTAACCAA |
| | GTATTTGTGTCCAAGAAT |
| | GTTTCCATCTTCTTTAAAA |
| | TCAATACCTTTTAACTCGA |
| | TTCTATTAACAAGGGTATC |
| | ACCGATCGCCTAGACAAC |
| | TCCTG (SEQ ID NO: 88) |
| GFPmut3_35_13,14-rev | AGTGTTGAGCGTAACCAA |
| | GTTTTTGAAGTTAACTTTG |
| | ATTCCATTCTTTTGTTTGT |
| | CTGCCATGATGTATACAT |
| | TGTGTGAGTTATAGTTGT |
| | ATTCCAGATCGCCTAGAC |
| | AACTCCTG (SEQ ID NO: 89) |
| GFPmut3_35_15,16-rev | AGTGTTGAGCGTAACCAA |
| | GTATCGCCAATTGGAGTA |
| | TTTTGTTGATAATGGTCT |
| | GCTAGTTGAACGCTTCCA |
| | TCTTCAATGTTGTGTCTA |
| | AGATCGCCTAGACAACT |
| | CCTG (SEQ ID NO: 90) |
| GFPmut3_35_17,18-rev | AGTGTTGAGCGTAACCA |
| | AGTTTGGGATCTTTCGA |
| | AAGGGCAGATTGTGTG |
| | GACAGGTAATGGTTGT |
| | CTGGTAAAAGGACAGGG |
| | CCGATCGCCTAGACAAC |
| | TCCTG (SEQ ID NO: 91) |
| GFPmut3_35_19,20-rev | AGTGTTGAGCGTAACCA |
| | AGTTATAGTTCATCCAT |
| | GCCATGTGTAATCCCAG |
| | CAGCTGTTACAAACTC |
| | AAGAAGGACCATGTGG |
| | TCTCTCTTTTCGGATCG |
| | CCTAGACAACTCCTG (SEQ ID |
| | NO: 92) |
| GFPmut3_35_0,21-rev- | AGTGTTGAGCGTAACC |
| bridge | AAGTGGTACCTGTACGC |
| | GAAAGCAGTCACCCTA |
| | TCCAGCCTCATGCGAC |
| | CGAGAAGAAGTAAGCT |
| | TTTATTTGGATCGCCTA |
| | GACAACTCCTG (SEQ ID NO: 93) |

Table 6 sets forth OLS Pool 1 oligonucleotide sequences for GFP35.

TABLE 7

| | |
|---|---|
| ygfJ-aspcr | AAGCAAGATTCTCGTCGGATccggacgact |
| | ttattacagcgaaggaaaggtatactg |
| | aaatttaAaaaacgtagttaaacgattg |
| | cgttcaaatatttaatccttccggcGATCC |
| | GAGATGTGCCTTACA (SEQ ID NO: 94) |
| recJ-aspcr | AAGCAAGATTCTCGTCGGATgggattgtac |
| | ccaatccacgctcttttttatagagaag |
| | atgacgTtaaattggccagatattgtcga |
| | tgataatttgcaggctgcggttgGATC |
| | CGAGATGTGCCTTACA (SEQ ID NO: 95) |
| argO-aspcr | AAGCAAGATTCTCGTCGGATctctggagg |
| | caagcttagcgcctctgttttatttttccat |
| | cagatagcgcTtaactgaacaaggct |
| | tgtgcatgagcaataccgtctctcGAT |
| | CCGAGATGTGCCTTACA (SEQ ID NO: 96) |
| yggU-aspcr | AAGCAAGATTCTCGTCGGATaatccgca |
| | acaaatcccgccagaaatcgcgg |
| | cgttaattaattaAgtatcctatgcaaa |
| | aagttgtcctcgcaaccggcaatgtcggta |
| | aGATCCGAGATGTGCCTTACA (SEQ ID |
| | NO: 97) |
| mutY-aspcr | AAGCAAGATTCTCGTCGGATgtggagc |
| | gtttgttacagcagttacgcactg |
| | gcgcgccggtttaAcgcgtgagtcg |
| | ataaagaggatgatttatgagcagaacgatt |
| | tttGATCCGAGATGTGCCTTACA (SEQ ID |
| | NO: 98) |
| glcC-aspcr | AAGCAAGATTCTCGTCGGATgccacca |
| | Tttgattcgctcggcggtgccgctg |
| | gagatgaacctgagttaActggta |
| | ttaaatctgcttttcatacaatcggtaacgct |
| | tgGATCCGAGATGTGCCTTACA (SEQ ID |
| | NO: 99) |
| yghQ-aspcr | AAGCAAGATTCTCGTCGGATactgagtca |
| | gccgagaagaatttccccgcttattcgcac |
| | cttccTtaaatcaggtcatacgcttcgagat |
| | acttaacgccaaacaccagcGA |
| | TCCGAGATGTGCCTTACA (SEQ ID |
| | NO: 100) |
| yghT-aspcr | AAGCAAGATTCTCGTCGGATtggttgatg |
| | Cagaaaaagcgattacggattttatga |
| | ccgcgcgtggttatcactaAtcaaaaat |
| | ggaaatgcccgatcgccaggaccgg |
| | gGATCCGAGATGTGCCTTACA (SEQ ID |
| | NO: 101) |
| ygiZ-aspcr | AAGCAAGATTCTCGTCGGATttctctgtc |
| | tatgagagccgttaaaacgactctcatag |
| | attttaTtaatagcaaaatataaaccgtcc |
| | ccaaaaaagccaccaaccacaa |
| | GATCCGAGATGTGCCTTACA (SEQ ID |
| | NO: 102) |
| yqiB-aspcr | AAGCAAGATTCTCGTCGGATagggtta |
| | acaggctttccaaatggtgtccttaggttt |
| | cacgacgTtaataaaccggaatcgc |
| | catcgctccatgtgctaaacagtatc |
| | gcGATCCGAGATGTGCCTTACA (SEQ ID |
| | NO: 103) |

Table 7 sets forth Control 1 oligos.

TABLE 8

| | |
|---|---|
| cat_fwd_*restore*-selctn | TCTAATCTAGCGCGACGTC |
| | TGCATCGTAAAGAAC |
| | ATTTTGAGGCATTTCAGTCAG |
| | TTGCTCAATGTACCTATAACC |
| | AGACCGTTCAGCTGGATATT |
| | ACGGCCTTTTTAAAG |
| | ATCATAGCGCCTCTTGTGG |
| | (SEQ ID NO: 104) |
| kan_fwd_*restore*-selctn | TCTAATCTAGCGCGACGTCTCG |
| | CGATTAAATTCCAACATGG |
| | ATGCTGATTTATATGGGTAT |
| | AAATGGGCTCGCGATAATGT |
| | CGGGCAATCAGGTGCGACA |
| | ATCTATCGCT |
| | GATCATAGCGCCTCTTGTGG |
| | (SEQ ID NO: 105) |
| malK_mut45_oligo-selctn | TCTAATCTAGCGCGACGTCTCC |
| | AAATGACATGTTTTCTGCTA |
| | CTGACAGGTGGGGATAGAG |
| | CGCTTAAGACTGAAACACC |
| | ATACCAACGCCGCGTTCTG |
| | CTGGCGGAGTGGATCATAG |
| | CGCCTCTTGTGG |
| | (SEQ ID NO: 106) |
| lacZ_oligo_m1_v1-selctn | TCTAATCTAGCGCGACGT |
| | CTGGAAACAGCTATGACCAT |
| | GATTACGGATTCACTGGCCG |
| | TCGTTTGACAACGTCGTGAC |
| | TGGGAAAACCCTGGCGTTA |
| | CCCAACTTAATCGGATCAT |
| | AGCGCCTCTTGTGG |
| | (SEQ ID NO: 107) |
| tolC_restore_oligo-selctn | TCTAATCTAGCGCGACGTCTA |
| | GCCTTTCTGGGTTCAGTTCG |
| | TTGAGCCAGGCCGAGAACC |
| | TGATGCAAGTTTATCAGCA |
| | AGCACGCCTTAGTAACCCG |
| | GAATTGCGTAAGGATCATAG |
| | CGCCTCTTGTGG |
| | (SEQ ID NO: 108) |

Table 8 depicts Control 2 oligos.

TABLE 9

| | |
|---|---|
| GFPmut3_20_0,1-for | GATAGGGTGACTGCTTTCGCGTACA |
| | GGTACCATGA (SEQ ID NO: 109) |
| GFPmut3_20_2,3-for | GTAAAGGAGAAGAACTTTTCACTGG |
| | AGTTGTCCCAATTCT |
| | (SEQ ID NO: 110) |
| GFPmut3_20_4,5-for | TGTTGAATTAGATGGTGATGTTAAT |
| | GGGCACAAATTTTCTGT (SEQ ID |
| | NO: 111) |
| GFPmut3_20_6,7-for | CAGTGGAGAGGGTGAAGGTGATGC |
| | AACATACGGAA (SEQ ID NO: 109) |
| GFPmut3_20_8,9-for | AACTTACCCTTAAATTTATTTGCAC |
| | TACTGGAAAACTACCTGT (SEQ ID |
| | NO: 112) |
| GFPmut3_20_10,11-for | TCCATGGCCAACACTTGTCACTACT |
| | TTCGGTTATGGT (SEQ |
| | ID NO: 113) |
| GFPmut3_20_12,13-for | GTTCAATGCTTTGCGAGATACCCAG |
| | ATCATATGAAACAG (SEQ ID |
| | NO: 114) |
| GFPmut3_20_14,15-for | CATGACTTTTTCAAGAGTGCCATGC |
| | CCGAAGGTTATG (SEQ ID NO: 115) |
| GFPmut3_20_16,17-for | TACAGGAAAGAACTATATTTTTCAA |
| | AGATGACGGGAACTACA (SEQ ID |
| | NO: 116) |
| GFPmut3_20_18,19-for | AGACACGTGCTGAAGTCAAGTTTG |
| | AAGGTGATACCCT (SEQ ID |
| | NO: 117) |
| GFPmut3_20_20,21-for | TGTTAATAGAATCGAGTTAAAAGGT |
| | ATTGATTTTAAAGAAGATGGA (SEQ |
| | ID NO: 118) |
| GFPmut3_20_22,23-for | AACATTCTTGGACACAAATTGGAAT |
| | ACAACTATAACTCACACAA (SEQ ID |
| | NO: 119) |
| GFPmut3_20_24,25-for | TGTATACATCATGGCAGACAAACAA |
| | AAGAATGGAATCAAAGTT (SEQ ID |
| | NO: 120) |
| GFPmut3_20_26,27-for | AACTTCAAAATTAGACACAACATT |
| | GAAGATGGAAGCGTTCA (SEQ ID |
| | NO: 121) |
| GFPmut3_20_28,29-for | ACTAGCAGACCATTATCAACAAAA |
| | TACTCCAATTGGCGAT (SEQ ID |
| | NO: 122) |
| GFPmut3_20_30,31-for | GGCCCTGTCCTTTTACCAGACAACC |
| | ATTACCTGTCC (SEQ ID NO: 123) |
| GFPmut3_20_32,33-for | ACACAATCTGCCCTTTCGAAAGATC |
| | CCAACGAAAAGA (SEQ ID NO: 124) |
| GFPmut3_20_34,35-for | GAGACCACATGGTCCTTCTTGAGTT |
| | TGTAACAGCTG (SEQ ID NO: 125) |
| GFPmut3_20_36,37-for | CTGGGATTACACATGGCATGGATGA |
| | ACTATACAAATAAAAG (SEQ ID |
| | NO: 126) |
| GFPmut3_20_38,39-for | CTTACTTCTTCTCGGTCGCATGAGG |
| | CTGATCAGCG (SEQ ID NO: 127) |
| GFPmut3_20_1,2-rev | GTGAAAAGTTCTTCTCCTTTACTCA |
| | TGGTACCTGTACGC (SEQ |
| | ID NO: 128) |
| GFPmut3_20_3,4-rev | TAACATCACCATCTAATTCAACAAG |
| | AATTGGGACAACTCCA (SEQ ID |
| | NO: 129) |
| GFPmut3_20_5,6-rev | CTTCACCCTCTCCACTGACAGAAA |
| | ATTTGTGCCCAT (SEQ ID NO: 130) |
| GFPmut3_20_7,8-rev | GCAAATAAATTTAAGGGTAAGTTT |
| | TCCGTATGTTGCATCAC (SEQ ID |
| | NO: 131) |
| GFPmut3_20_9,10-rev | CAAGTGTTGGCCATGGAACAGGT |
| | AGTTTTCCAGTAGT (SEQ ID |
| | NO: 132) |
| GFPmut3_20_11,12-rev | TCTCGCAAAGCATTGAACACCATA |
| | ACCGAAAGTAGTGA (SEQ ID |
| | NO: 133) |
| GFPmut3_20_13,14-rev | GCACTCTTGAAAAAGTCATGCTGT |
| | TTCATATGATCTGGGTA (SEQ ID |
| | NO: 134) |
| GFPmut3_20_15,16-rev | GAAAAATATAGTTCTTTCCTGTAC |
| | ATAACCTTCGGGCATG (SEQ ID |
| | NO: 135) |
| GFPmut3_20_17,18-rev | GACTTCAGCACGTGTCTTGTAGTT |
| | CCCGTCATCTTT (SEQ ID NO: 136) |
| GFPmut3_20_19,20-rev | CTTTTAACTCGATTCTATTAACAA |
| | GGGTATCACCTTCAAACTT (SEQ ID |
| | NO: 137) |
| GFPmut3_20_21,22-rev | CAATTTGTGTCCAAGAATGTTTCC |
| | ATCTTCTTTAAAATCAATAC (SEQ ID |
| | NO: 138) |
| GFPmut3_20_23,24-rev | TGTCTGCCATGATGTATACATTGT |
| | GTGAGTTATAGTTGTATTC (SEQ ID |
| | NO: 139) |
| GFPmut3_20_25,26-rev | ATGTTGTGTCTAATTTTGAAGTTA |
| | ACTTTGATTCCATTCTTTTGTT |
| | (SEQ ID NO: 140) |
| GFPmut3_20_27,28-rev | GTTGATAATGGTCTGCTAGTTGAA |
| | CGCTTCCATCTTCA (SEQ ID |
| | NO: 141) |
| GFPmut3_20_29,30-rev | GGTAAAAGGACAGGGCCATCGCC |
| | AATTGGAGTATTTT (SEQ ID |
| | NO: 142) |
| GFPmut3_20_31,32-rev | GAAAGGGCAGATTGTGTGGACA |
| | GGTAATGGTTGTCT (SEQ ID |
| | NO: 143) |
| GFPmut3_20_33,34-rev | AAGGACCATGTGGTCTCTCTTTT |
| | CGTTGGGATCTTTC (SEQ ID |
| | NO: 144) |
| GFPmut3_20_35,36-rev | TGCCATGTGTAATCCCAGCAGCT |
| | GTTACAAACTCAAG (SEQ ID |
| | NO: 145) |
| GFPmut3_20_37,38-rev | CGACCGAGAAGAAGTAAGCTTT |
| | TATTTGTATAGTTCATCCA (SEQ ID |
| | NO: 146) |
| GFPmut3_20_0,39-rev- | GAAAGCAGTCACCCTATCCGCT |
| bridge | GATCAGCCTCATG (SEQ |
| | ID NO: 147) |

Table 9 depicts IDT primers for GFP20

TABLE 10

| | |
|---|---|
| GFPfwd | GATAGGGTGACTGCTTTCGCGTACA (SEQ ID |
| | NO: 148) |
| GFPrev | CAGCCTCATGCGACCGAGAAGAAGT (SEQ ID |
| | NO: 149) |
| GFPfwd1 | GATCGGTACCATGAGTAAAGGAGAAGAACTTTT |
| | CACTGG (SEQ ID NO: 150) |
| GFPrev2 | GATCAAGCTTTTATTTGTATAGTTCATCCATGCC |
| | ATGTG (SEQ ID NO: 151) |
| GFPfwd3 | GATAGGGTGACTGCTTTC (SEQ ID NO: 152) |
| GFPrev3 | AAGCTTTTATTTGTATAGTTCATCCATGCCATGTG |
| | (SEQ ID NO: 153) |

Table 10 depicts GFP assembly primers.

The synthesized GFPmut3 sequence is as follows: GATAGGGTGACTGCTTTCGC GTACAGGTACCATGAGTAAAGGAGAAGAACTTTTCACTGGAGTTGTCCCA ATTCTTGTTGAATTAGATGGTGATGTTAATGGGCACAAATTTTCTGTCAGT GGAGAGGGTGAAGGTGATGCAACATACGGAAAACTTACCCTTAAATTTAT TTGCACTACTGGAAAACTACCTGTTCCATGGCCAACACTTGTCACTACTTT CGGTTATGGTGTTCAATGCTTTGCGAGATACCCAGATCATATGAAACAGC ATGACTTTTTCAAGAGTGCCATGCCCGAAGGTTATGTACAGGAAAGAACT ATATTTTTCAAAGATGACGGGAACTACAAGACACGTGCTGAAGTCAAGTT TGAAGGTGATACCCTTGTTAATAGAATCGAGTTAAAAGGTATTGATTTTAA AGAAGATGGAAACATTCTTGGACACAAATTGGAATACAACTATAACTCAC ACAATGTATACATCATGGCAGACAAACAAAAGAATGGAATCAAAGTTAAC TTCAAAATTAGACACAACATTGAAGATGGAAGCGTTCAACTAGCAGACCA TTATCAACAAAATACTCCAATTGGCGATGGCCCTGTCCTTTTACCAGACAA CCATTACCTGTCCACACAATCTGCCCTTTCGAAAGATCCCAACGAAAAGA GAGACCACATGGTCCTTCTTGAGTTTGTAACAGCTGCTGGGATTACACATG GCATGGATGAACTATACAAATAAAAGCTTACTTCTTCTCGGTCGCATGAG GCTG (SEQ ID NO:154).

### Plate Specific Primers

Florescent Protein Plate Primers: skpp-1-F (forward), ATATAGATGCCGTCCTAGCG (SEQ ID NO:155); skpp-1-R (reverse), AAGTATCTTTCCTGTGCCCA (SEQ ID NO:156). Antibodies Plate Primers: skpp-2-F, CCCTTTAATCAGATGCGTCG (SEQ ID NO:157); skpp-2-R, TGGTAGTAATAAGGGCGACC (SEQ ID NO:158).

### Fluorescent Protein Assembly Specific Primers

mTFP1-BtsI-20: skpp-202-F, AATCCTTGCGTCAATGGTTC (SEQ ID NO:159); skpp-202-R, GGGTTCTCGGATTTTACACG (SEQ ID NO:160). mCitrine-BtsI-20: skpp-203-F, TGTCGTGCCTCTTTATCTGT (SEQ ID NO:161); GCTTCGGTGTATCGGAAATG (SEQ ID NO:162). mApple-BtsI-20: skpp-204-F, ATTTAAACGGTGAGGTGTGC (SEQ ID NO:163); skpp-204-R, TATCGTTTCGCTGGCTATCA (SEQ ID NO:164).

### Fluorescent Protein Construction Primers

mTFP1-BtsI-20: skpp-102-F, TTTGCTTCAGTCAGATTCGC (SEQ ID NO:155); skpp-102-R, GTTCAATCACTGAATCCCGG (SEQ ID NO:165). mCitrine-BtsI-20: skpp-103-F, GTCGAGTCCTATGTAACCGT (SEQ ID NO:166); skpp-103-R, CAGGGGTCGTCATATCTTCA (SEQ ID NO:167). mApple-BtsI-20: skpp-104-F, GTAAGATGGAAGCCGGGATA (SEQ ID NO:168); skpp-104-R, CACCTCATAGAGCTGTGGAA (SEQ ID NO:169).

TABLE 10

| Use | FwdName | FwdSeq | RevName | RevSeq |
|---|---|---|---|---|
| trastuzumab-BtsI-20 | skpp-301-F | CTTAAACCGG | skpp-301-R | ATGCTACTCG |
| | | CCAACATACC | | TTCCTTTCGA |
| | | (SEQ ID NO: 170) | | (SEQ ID NO: 212) |
| Cetuximab-BtsI-20 | skpp-302-F | TGCTCTTTATT | skpp-302-R | TCTTATCGGT |
| | | CGTTGCGTC | | GCTTCGTTCT |
| | | (SEQ ID NO: 171) | | (SEQ ID NO: 213) |
| alemtuzumab-BtsI-20 | skpp-303-F | TGAGCCTTATG | skpp-303-R | GTCCGTTTTC |
| | | ATTTCCCGT | | CTGAATGAGC |
| | | (SEQ ID NO: 172) | | (SEQ ID NO: 214) |
| bevacizumab-BtsI-20 | skpp-304-F | CGTTCTAAACG | skpp-304-R | AGTCTGTCTT |
| | | GCTAGATGC | | TCCCCTTTCC |
| | | (SEQ ID NO: 173) | | (SEQ ID NO: 215) |
| ranibizumab-BtsI-20 | skpp-305-F | GTATCCGAAGC | skpp-305-R | CAGGTATGC |
| | | GTGGAGTAT | | GTAGGAGTCAA |
| | | (SEQ ID NO: 174) | | (SEQ ID NO: 216) |
| pertuzumab-BtsI-20 | skpp-306-F | CTTGTTATGGAC | skpp-306-R | TTAATGGCG |
| | | GAGTTGCC | | CGTTCATACTG |
| | | (SEQ ID NO: 175) | | (SEQ ID NO: 217) |
| naptumomab-BtsI-20 | skpp-307-F | CCAAAGATTCAA | skpp-307-R | ATTAGCCAT |
| | | CCGTCCTG | | TTCAGGACGGA |
| | | (SEQ ID NO: 176) | | (SEQ ID NO: 218) |
| tadocizumab-BtsI-20 | skpp-308-F | TATTCATGCTTG | skpp-308-R | ACTATGTAC |
| | | GACGGACT | | CGCTTGTTGGA |
| | | (SEQ ID NO: 177) | | (SEQ ID NO: 219) |
| efungumab-BtsI-20 | skpp-309-F | ATCGACAATGGT | skpp-309-R | TATGTCTCC |
| | | ATGGCTGA | | TAGCCACTCCT |
| | | (SEQ ID NO: 178) | | (SEQ ID NO: 220) |
| Abagovomab-BtsI-20 | skpp-310-F | GTCCTAGTGAG | skpp-310-R | CCGAAGAAT |
| | | GAATACCGG | | CGCAGATCCTA |
| | | (SEQ ID NO: 179) | | (SEQ ID NO: 221) |
| Motavizumab-BtsI- | skpp-311-F | TTAGATAGGTG | skpp-311-R | TAAGGTGCGT |
| 20 | | TGTAGGCGC | | ACTAGCTGAC |
| | | (SEQ ID NO: 180) | | (SEQ ID NO: 222) |
| bavituximab-BtsI-20 | skpp-312-F | TTCCGTTTATG | skpp-312-R | TCCTTGGAGT |
| | | CTTTCCAGC | | TTAGAGCGAG |
| | | (SEQ ID NO: 181) | | (SEQ ID NO: 223) |
| lexatumumab-BtsI-20 | skpp-313-F | GTATAGTTTGT | skpp-313-R | ATCAATCCCC |
| | | GCGGTGGTC | | TACACCTTCG |
| | | (SEQ ID NO: 182) | | (SEQ ID NO: 224) |
| ibalizumab-BtsI-20 | skpp-314-F | TCAGCCTTTCAT | skpp-314-R | TTCCTTGATA |
| | | TGATTGCG | | CCGTAGCTCG |
| | | (SEQ ID NO: 183) | | (SEQ ID NO: 225) |
| tenatumomab-BtsI-20 | skpp-315-F | AGGGTCGTGGTT | skpp-315-R | CGTTTCTTTC |
| | | AAAGGTAC | | CGGTCGTTAG |
| | | (SEQ ID NO: 184) | | (SEQ ID NO: 226) |
| canakinumab-BtsI-20 | skpp-316-F | TGCAAGTGTACA | skpp-316-R | GAACGGTGA |
| | | AATCCAGC | | TCCCTTTCCTA |
| | | (SEQ ID NO: 185) | | (SEQ ID NO: 227) |
| etaracizumab-BtsI-20 | skpp-317-F | CTTAAGGTTTGC | skpp-317-R | TGTTATAGCT |
| | | CCATTCCC | | TCCACGGTGT |
| | | (SEQ ID NO: 186) | | (SEQ ID NO: 228) |
| otelixizumab-BtsI-20 | skpp-318-F | TGGTTCGTTAGT | skpp-318-R | AGACGGGAT |
| | | CGATCTCC | | TTTACTGGGTC |
| | | (SEQ ID NO: 187) | | (SEQ ID NO: 229) |
| Panobacumab-BtsI- | skpp-319-F | TATTTTGTAGAG | skpp-319-R | TCTTTGCTTC |
| 20 | | CGTTCGCG | | GCAAGTCTTG |
| | | (SEQ ID NO: 188) | | (SEQ ID NO: 230) |
| gantenerumab-BtsI- | skpp-320-F | TTCTGTAAGTTT | skpp-320-R | CTAAACACCG |
| 20 | | CGTCGGGA | | CACCTCACTA |
| | | (SEQ ID NO: 189) | | (SEQ ID NO: 231) |
| milatuzumab-BtsI-20 | skpp-321-F | TTGACGTACGTA | skpp-321-R | GAACACAACT |
| | | GGTTCTCC | | ACACTGACGC |
| | | (SEQ ID NO: 190) | | (SEQ ID NO: 232) |
| veltuzumab-BtsI-20 | skpp-322-F | GAGATGAGTAGA | skpp-322-R | ATGGTCACTG |
| | | CGAGTGGG | | ACTCGCATTA |
| | | (SEQ ID NO: 191) | | (SEQ ID NO: 233) |
| Tanezumab-BtsI-20 | skpp-323-F | CTTTGGGCTTTCA | skpp-323-R | CAAAGATTTCT |
| | | GATGAGC | | GTCGGTCGG |
| | | (SEQ ID NO: 192) | | (SEQ ID NO: 234) |
| anrukinzumab-BtsI- | skpp-324-F | TGTCATATGCTAA | skpp-324-R | TGGCTACTTTCT |
| 20 | | CGTCCGT | | TAGCGGAA |
| | | (SEQ ID NO: 193) | | (SEQ ID NO: 235) |
| ustekinumab-BtsI-20 | skpp-325-F | TTGCGACATCACA | skpp-325-R | TACTTCGAGAC |
| | | ATTCTCG | | TTCATGCGT |
| | | (SEQ ID NO: 194) | | (SEQ ID NO: 236) |
| dacetuzumab-BtsI-20 | skpp-326-F | TCAGTATGGCGTC | skpp-326-R | ATGGCCCGACC |
| | | TTGAAGT | | TCTATTATG |
| | | (SEQ ID NO: 195) | | (SEQ ID NO: 237) |
| Alacizumab-BtsI-20 | skpp-327-F | TCATGTCGTGAC | skpp-327-R | TGGGTCTAGTG |
| | | CAGTAGAC | | AACTTCGTC |
| | | (SEQ ID NO: 196) | | (SEQ ID NO: 238) |
| tigatuzumab-BtsI-20 | skpp-328-F | AACTAACGGATTT | skpp-328-R | AACATATGTTGC |
| | | AAGCGCG | | TTCGTCCG |
| | | (SEQ ID NO: 197) | | (SEQ ID NO: 239) |
| Racotumomab-BtsI- | skpp-329-F | CATTTTCTGTTCC | skpp-329-R | TCGAGTTAGAT |
| 20 | | CCAGTGG | | TGTCACCCC |
| | | (SEQ ID NO: 198) | | (SEQ ID NO: 240) |
| conatumumab-BtsI- | skpp-330-F | ATTTGCCTAACCA | skpp-330-R | TCAGAGCTTTT |
| 20 | | CTCCACT | | CGGTACAGT |
| | | (SEQ ID NO: 199) | | (SEQ ID NO: 241) |
| afutuzumab-BtsI-20 | skpp-331-F | TGACTTATGAACC | skpp-331-R | GCCCAGGAGTA |
| | | TTTGCGC | | GTCGTTAAT |
| | | (SEQ ID NO: 200) | | (SEQ ID NO: 242) |
| oportuzumab-BtsI-20 | skpp-332-F | ATAGGATTAGCT | skpp-332-R | TCTGTGTTCCG |
| | | GATGGGCC | | ACTAAGGTC |
| | | (SEQ ID NO: 201) | | (SEQ ID NO: 243) |
| citatuzumab-BtsI-20 | skpp-333-F | TGAGATTCGGGA | skpp-333-R | TCTGTTGTTAG |
| | | CTATTCGG | | ACTCCGACC |
| | | (SEQ ID NO: 202) | | (SEQ ID NO: 244) |
| siltuximab-BtsI-20 | skpp-334-F | TTGGTTAGTACAC | skpp-334-R | GTACGTCTGA |
| | | GGGACTC | | ACTTGGGACT |
| | | (SEQ ID NO: 203) | | (SEQ ID NO: 245) |
| rafivirumab-BtsI-20 | skpp-335-F | ATTTGTGTATCG | skpp-335-R | AGACACGCGA |
| | | AGGCTCGT | | TTGTTTAACC |
| | | (SEQ ID NO: 204) | | (SEQ ID NO: 246) |
| Foravirumab-BtsI-20 | skpp-336-F | ATCGTTCCCCAT | skpp-336-R | CCGTTCGTTTT |
| | | CACATTCT | | GAGCACTTA |
| | | (SEQ ID NO: 205) | | (SEQ ID NO: 247) |
| Farletuzumab-BtsI-20 | skpp-337-F | ATTACCATGTTAT | skpp-337-R | AGGTTAGGGA |
| | | CGGGCGA | | ACGCAAGATT |
| | | (SEQ ID NO: 206) | | (SEQ ID NO: 248) |
| Elotuzumab-BtsI-20 | skpp-338-F | TCGGTGGATATG | skpp-338-R | CCAGACTGTGC |
| | | ACGTAACC | | TCGTTATCT |
| | | (SEQ ID NO: 207) | | (SEQ ID NO: 249) |
| necitumumab-BtsI-20 | skpp-339-F | GGTCAGATGGTT | skpp-339-R | AGTTGTTCTCT |
| | | TACATGCG | | ATCCGCGAT |
| | | (SEQ ID NO: 208) | | (SEQ ID NO: 250) |
| figitumumab-BtsI-20 | skpp-340-F | TCTCGTTCGAAAA | skpp-340-R | GATTAAATCT |
| | | TCATCGC | | CGCCGGTGAC |
| | | (SEQ ID NO: 209) | | (SEQ ID NO: 251) |
| Robatumumab-BtsI- | skpp-341-F | TGCAAATGTGAGG | skpp-341-R | TTGTAGTTTTC |
| 20 | | TAGCAAC | | GCTTGCGTT |
| | | (SEQ ID NO: 210) | | (SEQ ID NO: 252) |
| vedolizumab-BtsI-20 | skpp-342-F | AAAGTCAAAGTG | skpp-342-R | TGTGTTGCTC |
| | | CGTTTCGT | | TCTCATAGCC |
| | | (SEQ ID NO: 211) | | (SEQ ID NO: 253) |

Table 10 depicts antibody-specific primers.

TABLE 11

| Use | FwdName | FwdSeq | RevName | RevSeq |
|---|---|---|---|---|
| trastuzumab-BtsI-20 | skpp-101-F | GCTTATTCGT | skpp-101-R | TACTTTTGAT |
| | | GCCGTGTTAT | | TGCTGTGCCC |
| | | (SEQ ID NO: 254) | | (SEQ ID NO: 296) |
| Cetuximab-BtsI-20 | skpp-102-F | TTTGCTTCAG | skpp-102-R | GTTCAATCAC |
| | | TCAGATTCGC | | TGAATCCCGG |
| | | (SEQ ID NO: 255) | | (SEQ ID NO: 297) |
| alemtuzumab-BtsI-20 | skpp-103-F | GTCGAGTCCT | skpp-103-R | CAGGGGTCG |
| | | ATGTAACCGT | | TCATATCTTCA |
| | | (SEQ ID NO: 256) | | (SEQ ID NO: 298) |
| bevacizumab-BtsI-20 | skpp-104-F | GTAAGATGG | skpp-104-R | CACCTCATAG |
| | | AAGCCGGGATA | | AGCTGTGGAA |
| | | (SEQ ID NO: 257) | | (SEQ ID NO: 299) |
| ranibizumab-BtsI-20 | skpp-105-F | GGTGTCGCAA | skpp-105-R | CGGTTCCTAG |
| | | CATGATCTAC | | TCATGTTTGC |
| | | (SEQ ID NO: 258) | | (SEQ ID NO: 300) |
| pertuzumab-BtsI-20 | skpp-106-F | GTGCTAAGTC | skpp-106-R | TTGTACTAA |
| | | ACACTGTTGG | | TCTCGTCCCGG |
| | | (SEQ ID NO: 259) | | (SEQ ID NO: 301) |
| naptumomab-BtsI-20 | skpp-107-F | TCTAAACAGT | skpp-107-R | TTATGTTCA |
| | | TAGGCCCAGG | | CAACTGGCGTG |
| | | (SEQ ID NO: 260) | | (SEQ ID NO: 302) |
| tadocizumab-BtsI-20 | skpp-108-F | GTCTTTATAC | skpp-108-R | TGGAACTGA |
| | | TTGCCTGCCG | | TTTGGCCTTTG |
| | | (SEQ ID NO: 261) | | (SEQ ID NO: 303) |
| efungumab-BtsI-20 | skpp-109-F | CACCGCGATC | skpp-109-R | TATAGTTCC |
| | | AATACAACTT | | TCCCATGCACC |
| | | (SEQ ID NO: 262) | | (SEQ ID NO: 304) |
| Abagovomab-BtsI-20 | skpp-110-F | TTCGGATAGA | skpp-110-R | ACAATAGAC |
| | | CTCAGGAAGC | | AGACCCATGCA |
| | | (SEQ ID NO: 263) | | (SEQ ID NO: 305) |
| Motavizumab-BtsI-20 | skpp-111-F | CCATTGATAG | skpp-111-R | GAGTCGAGC |
| | | ATTCGCTCGC | | TAGCATAGGAG |
| | | (SEQ ID NO: 264) | | (SEQ ID NO: 306) |
| bavituximab-BtsI-20 | skpp-112-F | TTTTCTACTT | skpp-112-R | TTGTGGGAGC |
| | | TCCGGCTTGC | | TTCTTACCAT |
| | | (SEQ ID NO: 265) | | (SEQ ID NO: 307) |
| lexatumumab-BtsI-20 | skpp-113-F | ATGACTATTG | skpp-113-R | TCGTACGGGA |
| | | GGGTCGTACC | | ATGACCATAG |
| | | (SEQ ID NO: 266) | | (SEQ ID NO: 308) |
| ibalizumab-BtsI-20 | skpp-114-F | TCGACAATAG | skpp-114-R | AGACACAACG |
| | | TTGAGCCCTT | | TAGCCGATTA |
| | | (SEQ ID NO: 267) | | (SEQ ID NO: 309) |
| tenatumomab-BtsI-20 | skpp-115-F | GAGCCATGTG | skpp-115-R | CGGACTAAAG |
| | | AAATGTGTGT | | GATCGAGTCA |
| | | (SEQ ID NO: 268) | | (SEQ ID NO: 310) |
| canakinumab-BtsI-20 | skpp-116-F | CGTATACGTA | skpp-116-R | CATCGGATAAC |
| | | AGGGTTCCGA | | ACAAAGCGT |
| | | (SEQ ID NO: 269) | | (SEQ ID NO: 311) |
| etaracizumab-BtsI-20 | skpp-117-F | TTATGATGTC | skpp-117-R | GATGTATACTC |
| | | CGGATACCCG | | CACCGTGGT |
| | | (SEQ ID NO: 270) | | (SEQ ID NO: 312) |
| otelixizumab-BtsI-20 | skpp-118-F | TCTTAGAAATC | skpp-118-R | TGAGATATGTAC |
| | | CACGGGTCC | | CTGGTGCC |
| | | (SEQ ID NO: 271) | | (SEQ ID NO: 313) |
| Panobacumab-BtsI- | skpp-119-F | GAAGGGTGGA | skpp-119-R | ATTCTTGGGCC |
| 20 | | TCATCGTACT | | TATCGTTGT |
| | | (SEQ ID NO: 272) | | (SEQ ID NO: 314) |
| gantenerumab-BtsI- | skpp-120-F | GGCTGTTAGT | skpp-120-R | AAACCATATAC |
| 20 | | TTTAGAGCCG | | AGCCGTCGT |
| | | (SEQ ID NO: 273) | | (SEQ ID NO: 315) |
| milatuzumab-BtsI-20 | skpp-121-F | AGTGGTGTAG | skpp-121-R | TAGCTAAATCC |
| | | TGGCTTCTAC | | CACCCGATG |
| | | (SEQ ID NO: 274) | | (SEQ ID NO: 316) |
| veltuzumab-BtsI-20 | skpp-122-F | CTCAGAGGGA | skpp-122-R | GTGCGGTTACA |
| | | GTTCAACTGT | | GTTTTGACT |
| | | (SEQ ID NO: 275) | | (SEQ ID NO: 317) |
| Tanezumab-BtsI-20 | skpp-123-F | TTTGGCAGAT | skpp-123-R | GGGACTACATA |
| | | CATTAACGGC | | GGGTGACAG |
| | | (SEQ ID NO: 276) | | (SEQ ID NO: 318) |
| anrukinzumab-BtsI- | skpp-124-F | TATGATCTCC | skpp-124-R | CGTTGTCGTTC |
| 20 | | GTACACGAGC | | CAAAGAAGT |
| | | (SEQ ID NO: 277) | | (SEQ ID NO: 319) |
| ustekinumab-BtsI-20 | skpp-125-F | AGTGCCATGT | skpp-125-R | AGTCACACATA |
| | | TATCCCTGAA | | TACGGACCC |
| | | (SEQ ID NO: 278) | | (SEQ ID NO: 320) |
| dacetuzumab-BtsI-20 | skpp-126-F | TTATACATCTG | skpp-126-R | AGAGAACCCCT |
| | | GACGCCTCC | | ATTATGGCG |
| | | (SEQ ID NO: 279) | | (SEQ ID NO: 321) |
| Alacizumab-BtsI-20 | skpp-127-F | TCCTCGATTCT | skpp-127-R | TCGTTAGGCTA |
| | | CCAATCAGG | | AAACATGCG |
| | | (SEQ ID NO: 280) | | (SEQ ID NO: 322) |
| tigatuzumab-BtsI-20 | skpp-128-F | GCTTAACGCAT | skpp-128-R | TGATAGGTCGT |
| | | TTCAAGCAC | | TCAGCCTAC |
| | | (SEQ ID NO: 281) | | (SEQ ID NO: 323) |
| Racotumomab-BtsI- | skpp-129-F | CTTTTATGTTC | skpp-129-R | TCGGGACTTTC |
| 20 | | CTCGCAGGG | | ATAAGCACT |
| | | (SEQ ID NO: 282) | | (SEQ ID NO: 324) |
| conatumumab-BtsI- | skpp-130-F | GTGGGCGTTA | skpp-130-R | ATTTTATGCGT |
| 20 | | GCAAATTACA | | CCAGTTCGG |
| | | (SEQ ID NO: 283) | | (SEQ ID NO: 325) |
| afutuzumab-BtsI-20 | skpp-131-F | AGAGATTATT | skpp-131-R | AAGGCTGGTAT |
| | | AGGCGTGGGG | | TTCCCTTCA |
| | | (SEQ ID NO: 284) | | (SEQ ID NO: 326) |
| oportuzumab-BtsI-20 | skpp-132-F | TAGGATTACT | skpp-132-R | CATACTGTTGG |
| | | GCTCGGTGAC | | TTGCTAGGC |
| | | (SEQ ID NO: 285) | | (SEQ ID NO: 327) |
| citatuzumab-BtsI-20 | skpp-133-F | TCGCGTGAGT | skpp-133-R | ATATACTGGAT |
| | | GGTTCATATA | | TCCGCCGTT |
| | | (SEQ ID NO: 286) | | (SEQ ID NO: 328) |
| siltuximab-BtsI-20 | skpp-134-F | CAATAGATAC | skpp-134-R | ACTTATGAACC |
| | | CCACCCGTCA | | CTTGGCACT |
| | | (SEQ ID NO: 287) | | (SEQ ID NO: 329) |
| rafivirumab-BtsI-20 | skpp-135-F | ATATATCCGC | skpp-135-R | ATAGATGTATG |
| | | CGTTGTACGT | | CCGTTCGGT |
| | | (SEQ ID NO: 288) | | (SEQ ID NO: 330) |
| Foravirumab-BtsI-20 | skpp-136-F | CGAGAGTCTC | skpp-136-R | TCTCTGTTTTCC |
| | | CCACGATATC | | GCACTTTG |
| | | (SEQ ID NO: 289) | | (SEQ ID NO: 331) |
| Farletuzumab-BtsI-20 | skpp-137-F | ATTCAGTTGG | skpp-137-R | AGTTATTCGTCT |
| | | TCTTACGGGT | | TTCCCGGT |
| | | (SEQ ID NO: 290) | | (SEQ ID NO: 332) |
| Elotuzumab-BtsI-20 | skpp-138-F | GGATTGCAAC | skpp-138-R | TACAGGAATCT |
| | | GTCAGGAAAT | | CCACGAAGC |
| | | (SEQ ID NO: 297) | | (SEQ ID NO: 333) |
| necitumumab-BtsI-20 | skpp-139-F | GAATGTTGCA | skpp-139-R | CCTCGGGCTTG |
| | | GACTGGAAGG | | TTACTAGAT |
| | | (SEQ ID NO: 292) | | (SEQ ID NO: 334) |
| figitumumab-BtsI-20 | skpp-140-F | GTCCATGAAT | skpp-140-R | ATTCTTCCGTCC |
| | | ACAACACCGG | | AACGTACT |
| | | (SEQ ID NO: 293) | | (SEQ ID NO: 335) |
| Robatumumab-BtsI- | skpp-141-F | TCGAACAATT | skpp-141-R | TAATCATACGAG |
| 20 | | TGCGATACCC | | TGGGCCTC |
| | | (SEQ ID NO: 294) | | (SEQ ID NO: 336) |
| vedolizumab-BtsI-20 | skpp-142-F | AAGTGCACAT | skpp-142-R | AGTTGGTAGAAT |
| | | TTCGTTTCGA | | TGACCGGT |
| | | (SEQ ID NO: 295) | | (SEQ ID NO: 337) |

Table 11 depicts antibody construction primers.

TABLE 12
mTFP1
GGTACCATGGTGAGCAAGGGCGAGGAAACCACAATGGGCGTAATCAAG
CCCGACATGAAGATCAAGCTGAAGATGGAGGGCAACGTGAATGGCCAC
GCCTTCGTGATCGAGGGCGAGGGCGAGGGCAAGCCCTACGACGGCACC
AACACCATCAACCTGGAGGTGAAGGAGGGAGCCCCCCTGCCCTTCTCC
TACGACATTCTGACCACCGCGTTCGCCTACGGCAACAGGGCCTTCACC
AAGTACCCCGACGACATCCCCAACTACTTCAAGCAGTCCTTCCCCGAG
GGCTACTCTTGGGAGCGCACCATGACCTTCGAGGACAAGGGCATCGTG
AAGGTGAAGTCCGACATCTCCATGGAGGAGGACTCCTTCATCTACGAG
ATACACCTCAAGGGCGAGAACTTCCCCCCCAACGGCCCCGTGATGCAG
AAAAAGACCACCGGCTGGGACGCCTCCACCGAGAGGATGTACGTGCGC
GACGGCGTGCTGAAGGGCGACGTCAAGCACAAGCTGCTGCTGGAGGGC
GGCGGCCACCACCGCGTTGACTTCAAGACCATCTACAGGGCCAAGAAG
GCGGTGAAGCTGCCCGACTATCACTTTGTGGACCACCGCATCGAGATC
CTGAACCACGACAAGGACTACAACAAGGTGACCGTTTACGAGAGCGCC
GTGGCCCGCAACTCCACCGACGGCATGGACGAGCTGTACAAGTAAAAG
CTT (SEQ ID NO: 338)
mCitrine
GGTACCATGGTGAGCAAGGGCGAGGAGCTGTTCACCGGGGTGGTGCCC
ATCCTGGTCGAGCTGGACGGCGACGTAAACGGCCACAAGTTCAGCGTG
TCCGGCGAGGGCGAGGGCGATGCCACCTACGGCAAGCTGACCCTGAAG
TTCATCTGCACCACCGGCAAGCTGCCCGTGCCCTGGCCCACCCTCGTG
ACCACCTTCGGCTACGGCCTGATGTGCTTCGCCCGCTACCCCGACCAC
ATGAAGCAGCACGACTTCTTCAAGTCCGCCATGCCCGAAGGCTACGTC
CAGGAGCGCACCATCTTCTTCAAGGACGACGGCAACTACAAGACCCGC
GCCGAGGTGAAGTTCGAGGGCGACACCCTGGTGAACCGCATCGAGCTG
AAGGGCATCGACTTCAAGGAGGACGGCAACATCCTGGGGCACAAGCTG
GAGTACAACTACAACAGCCACAACGTCTATATCATGGCCGACAAGCAG
AAGAACGGCATCAAGGTGAACTTCAAGATCCGCCACAACATCGAGGAC
GGCAGCGTGCAGCTCGCCGACCACTACCAGCAGAACACCCCCATCGGC
GACGGCCCCGTGCTGCTGCCCGACAACCACTACCTGAGCTACCAGTCC
AAACTGAGCAAAGACCCCAACGAGAAGCGCGATCACATGGTCCTGCTG
GAGTTCGTGACCGCCGCCGGGATCACTCTCGGCATGGACGAGCTGTAC
AAGTAAAAGCTT (SEQ ID NO: 339)
mApple
GGTACCATGGTGAGCAAGGGCGAGGAGAATAACATGGCCATCATCAAG
GAGTTCATGCGCTTCAAGGTGCACATGGAGGGCTCCGTGAACGGCCAC
GAGTTCGAGATCGAGGGCGAGGGCGAGGGCCGCCCCTACGAGGCCTTT
CAGACCGCTAAGCTGAAGGTGACCAAGGGTGGCCCCCTGCCCTTCGCC
TGGGACATCCTGTCCCCTCAGTTCATGTACGGCTCCAAGGTCTACATT
AAGCACCCAGCCGACATCCCCGACTACTTCAAGCTGTCCTTCCCCGAG
GGCTTCAGGTGGGAGCGCGTGATGAACTTCGAGGACGGCGGCATTATT
CACGTTAACCAGGACTCCTCCCTGCAGGACGGCGTGTTCATCTACAAG
GTGAAGCTGCGCGGCACCAACTTCCCCTCCGACGGCCCCGTAATGCAG
AAAAAGACCATGGGCTGGGAGGCCTCCGAGGAGCGGATGTACCCCGAG
GACGGCGCCTTAAAGAGCGAGATCAAAAAGAGGCTGAAGCTGAAGGAC
GGCGGCCACTACGCCGCCGAGGTCAAGACCACCTACAAGGCCAAGAAG
CCCGTGCAGCTGCCCGGCGCCTACATCGTCGACATCAAGTTGGACATC
GTGTCCCACAACGAGGACTACACCATCGTGGAACAGTACGAACGCGCC
GAGGGCCGCCACTCCACCGGCGGCATGGACGAGCTGTACAAGTAAAAG
CTT (SEQ ID NO: 340)
trastuzumab
GGCCCAGCCGGCCAGGCGCGAAGTGCAGCTGGTGGAGTCAGGCGGTGG
ACTGGTGCAGCCAGGAGGTTCCCTGAGACTCTCATGCGCAGCAAGCGG
TTTTAATATCAAGGACACTTATATACACTGGGTGCGCCAAGCCCCCGG
AAAGGGTCTGGAGTGGGTGGCCAGAATATACCCCACAAACGGCTATAC
CAGGTACGCAGATTCAGTGAAGGGGAGATTCACCATAAGCGCTGACAC
ATCTAAGAATACTGCTTACCTGCAAATGAATTCCCTGAGGGCAGAGGA
TACAGCTGTTTATTACTGCAGCCGGTGGGGCGGAGATGGCTTTTACGC
CATGGACTATTGGGGGCAGGGAACCCTGGTCACCGTTTCCAGCGGTGG
GTCAGGGGGCAGCGGCGGCGCCAGCGGAGCAGGGAGCGGTGGAGGCGA
TATCCAAATGACACAGTCCCCCTCTAGCCTGAGCGCCAGCGTCGGTGA
CAGGGTGACCATTACATGCAGGGCCTCTCAGGATGTTAATACTGCCGT
TGCATGGTACCAGCAGAAGCCCGGGAAGGCACCAAAGCTGCTGATCTA
TTCCGCTTCCTTTCTGTACAGCGGAGTGCCTAGCAGGTTTTCCGGATC
TCGCAGCGGAACTGATTTTACACTCACCATCAGCAGCCTCCAACCTGA
GGATTTTGCCACCTATTATTGCCAGCAACACTACACCACTCCACCCAC
TTTCGGCCAGGGAACTAAGGTGGAAATAAAAGGGCCC
(SEQ ID NO: 341)
Cetuximab
GGCCCAGCCGGCCAGGCGCCAGGTTCAGCTCAAGCAGTCTGGACCCGG
ACTGGTGCAGCCCTCTCAGTCTCTCTCTATCACCTGCACAGTGTCTGG
TTTCTCTCTCACCAACTACGGGGTCCATTGGGTTCGGCAGTCCCCAGG
GAAAGGGCTCGAATGGCTGGGCGTGATCTGGTCCGGCGGCAATACCGA
CTACAACACCCCATTTACTTCCAGGCTGTCAATTAATAAGGACAATTC
TAAGAGCCAGGTCTTCTTTAAGATGAACTCTCTCCAGTCTAATGATAC
TGCCATCTACTACTGTGCCCGGGCACTCACATACTACGATTATGAATT
CGCTTACTGGGGCCAGGGCACCCTCGTCACCGTGAGCGCAGGAGGATC
TGCTGGCTCTGGGTCAAGCGGTGGCGCTTCCGGCTCAGGGGGAGACAT
CCTGCTCACCCAGAGCCCCGTGATTCTGTCCGTTAGCCCCGGAGAACG
CGTTTCTTTTAGCTGTCGCGCATCTCAGAGCATCGGTACCAACATTCA
CTGGTATCAGCAGCGGACCGACGGGAGCCCTCGCCTCCTGATAAAATA
TGCTTCTGAGTCAATTAGCGGTATCCCCTCCAGATTTAGCGGGAGCGG
TTCTGGGACCGATTTCACACTGAGCATCAACTCTGTGGAGTCTGAAGA
TATCGCTGATTATTACTGTCAGCAAAACAACAATTGGCCTACCACCTT
CGGCGCCGGCACCAAGCTGGAACTGAAAGGGCCC
(SEQ ID NO: 342)
alemtuzumab
GGCCCAGCCGGCCAGGCGCCAAGTTCAGCTCCAGGAGTCAGGTCCTGG
TCTGGTGAGACCATCCCAGACCCTCTCTCTCACTTGTACCGTTTCCGG
CTTCACATTCACCGATTTCTATATGAACTGGGTTAGGCAACCACCAGG
CCGGGGGCTGGAATGGATCGGTTTTATCAGAGATAAAGCCAAGGGATA
TACTACTGAGTACAACCCCTCTGTGAAGGGTCGGGTGACCATGCTGGT
TGACACAAGCAAGAATCAATTTTCACTCCGGCTGTCATCTGTGACAGC
TGCTGATACAGCAGTTTATTATTGCGCAAGGGAAGGACATACTGCCGC
TCCTTTCGACTATTGGGGCCAGGGTTCACTCGTCACAGTCTCTTCAGG
TGGGGCCGGCTCAGGAGCCGGGAGCGGGTCATCTGGAGCCGGCTCCGG
GGATATCCAGATGACCCAGTCACCCTCTTCACTCAGCGCCAGCGTGGG
CGATCGCGTTACCATCACATGCAAAGCTTCTCAGAACATTGACAAATA
CCTGAATTGGTACCAACAGAAGCCCGGCAAGGCCCCCAAACTCCTCAT
ATACAATACAAACAATCTGCAGACCGGCGTGCCATCCCGCTTCTCAGG
ATCAGGCAGCGGCACTGACTTTACTTTCACAATCAGCAGCCTGCAACC
AGAGGACATCGCCACATATTACTGTCTCCAGCATATCTCCCGCCCTCG
GACATTCGGCCAAGGTACAAAGGTGGAGATTAAAGGGCCC
(SEQ ID NO: 343)
bevacizumab
GGCCCAGCCGGCCAGGCGCGAAGTGCAACTGGTTGAAAGCGGTGGGGG
CCTGGTGCAGCCTGGTGGATCACTGAGACTCTCCTGCGCCGCCAGCGG
TTACACCTTCACCAACTATGGTATGAATTGGGTTAGACAAGCACCTGG
AAAGGGACTGGAGTGGGTTGGCTGGATAAATACATATACAGGCGAGCC
AACATATGCAGCTGACTTTAAGCGGAGGTTTACCTTCTCACTGGACAC
ATCCAAGTCTACTGCTTACCTGCAGATGAACTCACTCCGGGCTGAGGA
TACAGCCGTTTACTATTGCGCCAAGTATCCCCATTACTATGGTTCCAG
CCACTGGTACTTCGATGTCTGGGGCCAGGGAACTCTGGTGACTGGGGG
GTCCGGGGGCTCCGGAGGGGCCTCCGGAGCAGGATCCGGCGGAGGTGA
CATACAGATGACCCAGTCTCCATCCTCTCTGAGCGCCTCTGTGGGCGA
TCGCGTCACTATTACCTGTTCTGCATCTCAGGATATTAGCAACTATCT
GAATTGGTATCAGCAGAAGCCAGGTAAGGCACCAAAAGTTCTGATCTA
CTTCACAAGCTCTCTGCATTCCGGGGTGCCCTCACGCTTCTCTGGTTC
CGGCTCCGGGACAGATTTCACACTCACAATTTCCTCTCTGCAGCCCGA
AGATTTTGCAACTTACTACTGTCAGCAGTATTCTACAGTGCCATGGAC
TTTCGGACAGGGAACCAAGGTCGAGATTAAAGGGCCC
(SEQ ID NO: 344)
ranibizumab
GGCCCAGCCGGCCAGGCGCGAAGTTCAGCTGGTTGAAAGCGGAGGTGG
ACTCGTGCAGCCCGGTGGGTCCCTGAGGCTCTCCTGCGCCGCTAGCGG
ATATGATTTCACTCACTACGGTATGAATTGGGTCCGGCAGGCTCCCGG
CAAAGGTCTGGAATGGGTTGGCTGGATCAACACTTATACTGGGGAGCC
TACCTACGCCGCCGATTTCAAGAGGCGCTTTACTTTCTCACTCGATAC
CTCCAAATCCACAGCCTATCTGCAAATGAATTCCCTGCGCGCCGAAGA
TACCGCAGTCTACTATTGTGCCAAGTATCCCTACTATTATGGGACATC
TCACTGGTACTTCGACGTGTGGGGGCAAGGGACTCTCGTCACTGTGTC
TAGCGGGGGTAGCGCTGGGTCCGGCAGCAGCGGTGGGGCAAGCGGTAG
CGGGGGCGACATTCAGCTGACACAAAGCCCCTCATCCCTGAGCGCTTC
AGTGGGGGACCGCGTGACCATCACCTGTTCCGCCTCCCAGGACATCTC
AAACTACCTGAACTGGTACCAACAAAAACCTGGTAAAGCCCCTAAAGT
TCTGATTTACTTCACAAGCTCTCTCCACTCCGGCGTCCCTTCTAGGTT
TTCTGGTAGCGGTAGCGGAACAGATTTCACTCTGACAATTAGCTCCCT
CCAGCCTGAGGATTTTGCCACTTACTATTGTCAGCAGTATTCCACAGT
GCCCTGGACTTTTGGGCAGGGCACCAAGGTCGAAATCAAGGGGCCC
(SEQ ID NO: 345)
pertuzumab
GGCCCAGCCGGCCAGGCGCGAGGTCCAGCTGGTCGAGAGCGGCGGCGG
GCTGGTTCAACCCGGGGGCTCCCTGCGGCTGTCATGTGCCGCCAGCGG
CTTCACCTTTACTGATTACACAATGGACTGGGTGAGGCAGGCCCCAGG
AAAAGGCCTGGAATGGGTTGCCGACGTGAATCCTAATTCCGGGGGTTC
AATTTACAATCAGCGCTTTAAGGGCCGGTTCACCCTGTCAGTCGACAG
GAGCAAGAATACACTCTATCTCCAGATGAACTCCCTCCGCGCTGAGGA
TACCGCCGTCTATTATTGTGCCCGCAATCTGGGTCCCTCTTTTTACTT
TGACTATTGGGGCCAAGGGACCCTGGTCACCGTCTCTAGCGCCGGTGG
CTCAGGAGGAAGCGGTGGCGCCTCTGGGGCTGGCAGCGGAGGAGGCGA
CATTCAGATGACACAGAGCCCTAGCTCTCTCTCCGCTAGCGTGGGGGA
CAGGGTTACCATAACTTGCAAGGCAAGCCAAGATGTCTCTATTGGTGT
TGCTTGGTACCAGCAAAAGCCTGGAAAGGCTCCTAAACTGCTGATATA
CTCCGCCAGCTACAGGTATACAGGCGTGCCATCCCGGTTCTCAGGTTC
CGGCTCAGGAACAGATTTTACTCTCACCATTTCCAGCCTGCAACCCGA
GGACTTCGCCACATACTATTGCCAGCAGTATTATATATATCCTTACAC
TTTTGGTCAGGGTACTAAAGTGGAGATTAAAGGGCCC
(SEQ ID NO: 346)
naptumomab
GGCCCAGCCGGCCAGGCGCGAGGTGCAGCTCCAACAATCTGGGCCTGA
TCTGGTTAAGCCAGGCGCTTCTGTGAAAATTTCCTGTAAGGCTTCAGG
CTACAGCTTCACTGGCTATTATATGCATTGGGTGAAACAGTCTCCAGG
AAAGGGCCTGGAGTGGATTGGGCGGATCAATCCCAACAATGGAGTCAC
CCTCTACAATCAAAAATTCAAAGATAAAGCTACACTGACCGTCGATAA
AAGCTCAACAACAGCCTACATGGAGCTGAGATCCCTCACCTCCGAGGA
CAGCGCTGTCTACTACTGCGCCAGGTCCACAATGATTACCAATTATGT
GATGGACTACTGGGGTCAGGGAACCTCAGTGACCGTTAGCTCTGGCGG
GTCCGCAGGTAGCGGCTCATCCGGCGGCGCATCCGGGAGCGGAGGGTC
TATTGTCATGACACAGACCCCCACTTCCCTCCTGGTCTCTGCTGGCGA
CAGAGTCACAATCACTTGCAAGGCTAGCCAGAGCGTTTCAAACGACGT
GGCATGGTATCAACAGAAACCCGGCCAATCCCCCAAACTGCTGATTTC
TTACACATCATCCAGATACGCCGGTGTGCCCGATAGGTTTTCTGGTTC
AGGGTATGGAACTGACTTCACTCTCACTATCTCTAGCGTTCAGGCTGA
AGACGCTGCCGTCTACTTCTGCCAGCAAGACTACAACTCTCCTCCTAC
ATTCGGCGGGGGCACAAAGCTGGAGATCAAAGGGCCC
(SEQ ID NO: 347)
tadocizumab
GGCCCAGCCGGCCAGGCGCCAGGTGCAGCTGGTGCAGTCCGGAGCCGA
GGTCAAGAAGCCCGGATCTTCCGTCAAAGTCAGCTGCAAAGCTTCCGG
TTATGCATTCACTAACTACCTCATCGAGTGGGTCCGCCAGGCTCCAGG
ACAGGGACTGGAGTGGATTGGAGTGATCTACCCTGGATCAGGAGGCAC
AAATTATAACGAGAAGTTTAAGGGCAGAGTCACTCTGACCGTCGATGA
ATCCACAAATACAGCTTACATGGAGCTGTCATCACTCCGGAGCGAGGA
CACAGCAGTTTATTTTTGCGCACGCCGCGATGGCAATTACGGGTGGTT
CGCCTATTGGGGGCAGGGTACTCTCGTCACCGTGTCATCAGGTGGGGC
TGGCTCCGGGGCAGGTTCTGGCTCCTCCGGAGCTGGTTCAGGAGACAT
CCAGATGACCCAGACACCCTCCACTCTCTCTGCTTCTGTGGGAGACAG
AGTCACAATCAGCTGCCGGGCTTCCCAGGATATAAACAACTACCTGAA
CTGGTACCAGCAGAAGCCTGGGAAGGCCCCCAAGCTGCTGATCTACTA
TACATCCACTCTGCACAGCGGAGTTCCTAGCCGCTTCAGCGGATCCGG
TAGCGGGACCGACTATACCCTGACCATCTCAAGCCTGCAGCCCGATGA
CTTCGCCACATACTTCTGTCAGCAGGGAAACACCCTCCCATGGACATT
CGGTCAAGGAACTAAAGTTGAGGTTAAAGGGCCC
(SEQ ID NO: 348)
efungumab
GGCCCAGCCGGCCAGGCGCGAAGTTCAACTGGTTGAGAGCGGTGCCGA
GGTGAAGAAGCCTGGAGAGTCTCTGAGAATTAGCTGTAAGGGCTCTGG
CTGCATCATCTCATCTTATTGGATTTCATGGGTTAGACAGATGCCCGG
CAAAGGACTGGAATGGATGGGCAAGATAGACCCTGGTGACTCCTACAT
CAATTATTCCCCTTCTTTTCAGGGGCATGTCACAATCTCCGCAGACAA
GAGCATCAACACAGCATATCTCCAGTGGAATTCACTGAAAGCCTCCGA
CACAGCCATGTACTATTGCGCAAGAGGAGGGAGGGACTTCGGAGACTC
TTTTGACTACTGGGGGCAGGGGACTCTGGTGACAGTGTCTAGCGGCGG
GTCAGGAGGATCCGGTGGAGCCTCTGGCGCTGGAAGCGGCGGCGGAGA
TGTGGTCATGACTCAATCCCCTTCCTTTCTGTCAGCATTCGTGGGCGA
TAGGATCACTATTACTTGTCGCGCCTCTTCTGGCATCTCCAGATATCT
GGCTTGGTACCAGCAAGCTCCCGGAAAGGCCCCTAAGCTGCTCATATA
TGCCGCCTCCACCCTCCAGACTGGAGTGCCCAGCCGGTTTAGCGGTAG
CGGTTCCGGTACCGAGTTTACCCTCACCATTAACTCTCTGCAGCCAGA
AGACTTCGCCACATATTACTGTCAACACCTCAACTCCTATCCTCTCAC
TTTCGGCGGCGGGACCAAAGTCGATATTAAGGGGCCC
(SEQ ID NO: 349)
Abagovomab
GGCCCAGCCGGCCAGGCGCCAAGTTAAACTGCAGGAGAGCGGAGCCGA
ACTCGCCAGACCCGGAGCTTCTGTGAAACTGAGCTGCAAAGCTTCTGG
CTATACTTTTACCAATTATTGGATGCAATGGGTGAAGCAGAGGCCAGG
ACAGGGACTGGACTGGATCGGAGCTATCTATCCTGGAGACGGCAATAC
TCGGTACACACACAAATTTAAGGGGAAAGCTACCCTGACCGCTGATAA
GTCATCATCTACCGCCTACATGCAGCTGAGCTCCCTGGCTTCAGAGGA
CAGCGGCGTTTACTATTGCGCACGCGGCGAGGGAAACTATGCATGGTT
TGCATACTGGGGGCAGGGGACCACCGTGACTGTGTCCTCAGGGGGGAG
CGCTGGTAGCGGTTCCAGCGGCGGGGCCAGCGGTTCCGGGGGGGACAT
CGAGCTCACTCAGTCTCCTGCAAGCCTGTCAGCATCAGTTGGGGAGAC
AGTTACCATCACCTGCCAGGCATCCGAAAATATATACAGCTACCTCGC
ATGGCATCAGCAAAAGCAGGGTAAAAGCCCTCAGCTCCTGGTTTATAA
TGCTAAAACCCTGGCTGGAGGCGTCTCTTCAAGATTTAGCGGGAGCGG
CTCCGGGACCCACTTCTCACTGAAAATAAAGTCCCTGCAACCAGAGGA
TTTTGGTATTTACTATTGTCAGCACCACTACGGCATACTCCCAACCTT
CGGAGGGGGAACTAAGCTGGAAATCAAGGGGCCC
(SEQ ID NO: 350)
Motavizumab
GGCCCAGCCGGCCAGGCGCCAGGTTACCCTGCGCGAGAGCGGGCCTGC
TCTGGTGAAACCCACTCAGACCCTGACTCTGACCTGCACATTCTCTGG
CTTTTCCCTCTCTACTGCCGGAATGTCAGTGGGATGGATCCGCCAGCC
TCCTGGCAAAGCTCTGGAGTGGCTCGCTGATATTTGGTGGGACGATAA
AAAGCATTATAATCCATCTCTGAAGGACCGCCTCACCATCAGCAAGGA
CACTAGCAAGAATCAGGTGGTTCTCAAGGTGACCAATATGGACCCAGC
TGATACCGCTACCTACTACTGTGCCAGGGACATGATCTTCAACTTCTA
TTTTGACGTGTGGGGTCAGGGCACCACCGTCACCGTTAGCTCTGGGGG
AGCCGGTAGCGGGGCCGGGAGCGGGAGCAGCGGCGCAGGCTCTGGAGA
TATACAGATGACTCAGAGCCCCTCTACCCTGTCTGCTTCCGTGGGCGA
CCGGGTCACCATCACATGCTCCGCCTCTAGCCGCGTCGGTTATATGCA
TTGGTACCAGCAGAAGCCCGGCAAGGCACCCAAACTCCTCATTTATGA
CACCTCCAAGCTGGCCTCTGGAGTTCCCTCTCGGTTTTCCGGAAGCGG
TAGCGGCACCGAGTTCACACTGACCATCTCCTCTCTCCAGCCAGATGA
TTTCGCCACATATTATTGCTTCCAGGGCAGCGGGTATCCTTTTACATT
TGGTGGGGGAACTAAAGTGGAGATCAAAGGGCCC
(SEQ ID NO: 351)
bavituximab
GGCCCAGCCGGCCAGGCGCGAGGTGCAACTCCAGCAGTCTGGTCCCGA
GCTGGAGAAGCCCGGCGCCAGCGTGAAGCTGTCATGTAAAGCCAGCGG
GTACTCATTCACTGGCTATAATATGAACTGGGTGAAACAGTCACATGG
TAAGAGCCTGGAATGGATCGGCCATATTGACCCCTATTACGGTGACAC
TTCTTATAACCAAAAATTCAGGGGTAAGGCCACCCTGACCGTGGACAA
ATCTAGCAGCACAGCCTATATGCAGCTCAAATCCCTGACATCAGAAGA
CAGCGCTGTTTATTATTGTGTGAAAGGCGGGTACTACGGTCATTGGTA
TTTCGACGTGTGGGGCGCCGGGACCACTGTGACTGTGTCCTCTGGCGG
ATCTGGCGGCTCTGGCGGGGCCTCCGGAGCCGGATCTGGGGGCGGCGA
CATTCAGATGACACAATCACCATCTTCTCTGTCCGCTTCCCTGGGTGA
GCGCGTCTCCCTCACATGCCGGGCTTCTCAGGACATAGGCAGCTCCCT
CAACTGGCTGCAACAGGGTCCAGACGGTACTATCAAGCGGCTCATTTA
TGCTACCTCTAGCCTGGATTCAGGCGTGCCCAAAAGGTTTTCTGGATC
TCGGTCCGGCTCAGACTATTCCCTCACTATTTCTTCTCTCGAAAGCGA
GGATTTCGTGGACTATTACTGTCTGCAGTACGTGAGCTCACCTCCTAC
TTTCGGGGCAGGCACCAAACTCGAACTGAAGGGGCCC
(SEQ ID NO: 352)
lexatumumab
GGCCCAGCCGGCCAGGCGCGAAGTTCAGCTGGTCCAGTCAGGAGGAGG
GGTCGAACGGCCCGGCGGATCTCTGCGGCTGTCCTGCGCCGCCAGCGG
CTTCACATTCGATGATTACGGTATGAGCTGGGTTAGACAAGCTCCAGG
GAAAGGACTGGAGTGGGTGTCCGGCATCAATTGGAACGGTGGCAGCAC
AGGCTATGCTGATAGCGTCAAGGGCAGAGTTACAATCAGCAGAGACAA
TGCCAAGAACTCTCTGTATCTCCAGATGAACTCCCTGAGGGCTGAAGA
TACCGCAGTCTATTATTGCGCCAAAATTCTGGGAGCCGGAAGAGGATG
GTACTTTGATCTCTGGGGGAAAGGAACTACAGTCACAGTGTCTGGGGG
CAGCGCAGGCAGCGGCTCCAGCGGCGGGGCTTCCGGATCAGGAGGGTC
CTCCGAGCTCACTCAGGACCCAGCTGTGTCTGTCGCCCTCGGGCAGAC
TGTGCGGATCACTTGTCAGGGAGATTCCCTCCGCTCCTATTATGCCTC
CTGGTACCAGCAGAAACCTGGCCAGGCCCCCGTGCTGGTCATCTACGG
CAAAAATAATCGCCCATCAGGCATTCCCGACCGGTTTAGCGGATCTTC
TTCCGGGAATACTGCCTCTCTGACAATTACTGGTGCCCAAGCTGAGGA
TGAGGCCGATTACTACTGTAACAGCCGCGACAGCTCAGGAAACCACGT
GGTGTTCGGGGGCGGAACTAAGCTCACCGTGCTGGGGCCC
(SEQ ID NO: 353)
ibalizumab
GGCCCAGCCGGCCAGGCGCCAGGTGCAGCTGCAACAATCCGGCCCCGA
GGTTGTGAAACCAGGCGCCTCTGTGAAGATGTCTTGCAAGGCCTCAGG
CTATACATTCACCAGCTATGTGATTCACTGGGTGCGCCAGAAACCAGG
ACAGGGTCTCGATTGGATTGGCTATATTAACCCTTACAATGATGGTAC
AGACTATGACGAGAAGTTTAAAGGCAAGGCCACACTGACAAGCGATAC
CTCTACTAGCACCGCCTATATGGAGCTCAGCTCCCTCCGGTCAGAAGA
CACCGCTGTGTATTATTGTGCCAGAGAAAAAGATAATTATGCTACAGG
CGCTTGGTTCGCCTACTGGGGACAGGGGACTCTCGTGACTGTGTCAAG
CGGTGGAGCCGGGTCCGGCGCCGGCTCTGGTTCCAGCGGGGCCGGTTC
CGGGGACATTGTGATGACCCAGTCTCCAGATAGCCTGGCTGTGTCTCT
GGGCGAGAGGGTGACAATGAATTGTAAGTCCTCACAAAGCCTCCTGTA
TTCTACCAATCAGAAGAACTACCTGGCTTGGTATCAACAGAAGCCAGG
CCAATCTCCCAAGCTCCTCATTTATTGGGCTTCCACAAGGGAGTCCGG
CGTGCCAGACCGGTTTAGCGGATCCGGCTCCGGCACTGATTTCACCCT
CACCATCAGCTCCGTTCAAGCCGAAGATGTGGCCGTCTACTACTGCCA
GCAATATTATTCCTATCGCACCTTTGGCGGAGGGACTAAACTGGAGAT
TAAGGGGCCC (SEQ ID NO: 354)
tenatumomab
GGCCCAGCCGGCCAGGCGCGAGATCCAACTCCAGCAGTCTGGACCTGA
GCTGGTGAAGCCAGGTGCCTCTGTGAAGGTGTCATGCAAAGCTTCCGG
CTATGCATTTACATCTTACAATATGTATTGGGTGAAGCAATCACATGG
CAAGAGCCTGGAGTGGATTGGCTATATTGATCCATATAATGGCGTGAC
CTCTTACAACCAGAAATTCAAGGGGAAGGCTACCCTCACAGTTGACAA
GTCTTCTTCTACTGCCTATATGCACCTCAATTCACTGACATCTGAGGA
CTCTGCCGTGTATTATTGCGCTAGGGGTGGAGGAAGCATCTACTATGC
CATGGACTATTGGGGACAAGGGACCAGCGTGACTGTCTCAAGCGGCGG
CTCTGGCGGCAGCGGCGGCGCCAGCGGCGCAGGCTCCGGGGGGGGAGA
TATTGTGATGACACAGGCCGCACCTTCCGTGCCTGTGACCCCTGGGGA
GTCAGTGAGCATCAGCTGCCGCTCCTCCAAGTCCCTGCTGCATTCCAA
TGGCAATACCTATCTCTATTGGTTCCTCCAGAGACCAGGACAATCCCC
ACAGCTGCTGATCTACAGAATGTCCAACCTCGCATCTGGAGTCCCTGA
CCGGTTCTCAGGCAGCGGTAGCGGCACCGCATTTACTCTGCGGATTTC
TAGGGTGGAGGCCGAAGATGTGGGTGTGTACTACTGTATGCAACACCT
GGAGTATCCCCTGACTTTTGGAGCCGGAACCAAGCTCGAACTGAAGGG
GCCC (SEQ ID NO: 355)
canakinumab
GGCCCAGCCGGCCAGGCGCCAGGTGCAACTCGTGGAATCTGGAGGCGG
CGTCGTGCAGCCCGGGAGGTCTCTGCGGCTGTCATGTGCAGCTTCAGG
CTTCACTTTCAGCGTCTATGGTATGAACTGGGTGAGACAGGCACCTGG
AAAAGGACTCGAATGGGTGGCCATCATCTGGTACGACGGCGACAACCA
ATACTACGCCGACTCCGTCAAGGGGAGATTCACAATTTCACGCGATAA
CTCCAAAAATACACTGTACCTCCAGATGAACGGCCTGAGAGCTGAGGA
CACAGCCGTTTATTACTGTGCCAGGGACCTCCGGACCGGACCCTTCGA
CTATTGGGGACAGGGGACACTGGTCACAGTGTCAAGCGCTTCCGGAGG
GTCTGCAGGGTCCGGATCCAGCGGGGGGGCTTCAGGGAGCGGAGGGGA
GATCGTTCTGACTCAGTCTCCAGACTTTCAGTCTGTCACACCAAAGGA
AAAGGTCACCATCACTTGCCGGGCCTCACAATCCATCGGTTCTAGCCT
GCACTGGTATCAGCAGAAACCAGACCAGTCCCCCAAGCTGCTCATCAA
GTACGCTTCACAGTCTTTCAGCGGCGTCCCATCCAGGTTCTCCGGCTC
CGGTTCCGGCACAGACTTCACTCTGACCATCAATAGCCTCGAAGCTGA
AGACGCTGCTGCTTATTACTGTCACCAAAGCAGCTCTCTGCCCTTTAC
TTTTGGTCCTGGCACAAAGGTGGACATTAAGGGGCCC
(SEQ ID NO: 356)
etaracizumab
GGCCCAGCCGGCCAGGCGCCAGGTGCAGCTGGTGGAAAGCGGTGGCGG
TGTCGTGCAGCCCGGCCGCAGCCTGAGACTCTCCTGCGCTGCATCAGG
TTTTACATTTTCTAGCTACGATATGTCTTGGGTCCGGCAGGCACCAGG
AAAGGGGCTGGAGTGGGTGGCTAAAGTTTCTTCCGGAGGGGGGAGCAC
CTACTATCTCGACACTGTTCAGGGCCGGTTCACTATATCCCGGGACAA
TTCTAAGAATACACTGTACCTGCAGATGAATTCTCTGAGGGCAGAAGA
TACCGCTGTGTACTATTGTGCACGGCATCTGCACGGATCCTTCGCTTC
CTGGGGACAGGGCACTACTGTCACCGTTTCTAGCGGCGGTGCTGGATC
TGGAGCTGGATCAGGGTCCTCTGGAGCTGGCTCAGGTGAGATCGTGCT
GACCCAAAGCCCTGCTACCCTGAGCCTCTCCCCAGGAGAGCGGGCAAC
ACTGTCTTGTCAGGCATCTCAATCAATTAGCAACTTCCTGCATTGGTA
CCAACAGCGGCCAGGCCAAGCCCCTAGGCTGCTCATTAGATACAGGTC
CCAATCAATTAGCGGAATACCAGCCAGGTTTTCCGGCTCTGGATCCGG
TACCGACTTCACCCTCACCATCTCTTCCCTGGAACCCGAAGACTTCGC
CGTGTATTACTGTCAGCAGTCTGGGTCTTGGCCTCTGACATTCGGAGG
TGGAACTAAAGTGGAAATCAAAGGGCCC
(SEQ ID NO: 357)
otelixizumab
GGCCCAGCCGGCCAGGCGCGAAGTGCAGCTGCTGGAAAGCGGCGGCGG
GCTGGTCCAGCCCGGCGGATCCCTGAGACTGTCATGTGCCGCCAGCGG
TTTCACTTTTAGCTCATTTCCAATGGCCTGGGTTCGGCAGGCACCAGG
AAAAGGCCTCGAATGGGTGTCCACAATATCAACTTCTGGCGGTAGAAC
ATACTATAGGGACTCCGTGAAGGGCAGATTTACCATTTCCCGGGATAA
TAGCAAGAATACACTGTATCTGCAGATGAATTCACTGAGGGCTGAAGA
TACAGCCGTGTATTATTGCGCCAAATTTCGCCAGTATTCTGGCGGCTT
TGACTACTGGGGACAGGGCACTCTCGTCACAGTGAGCTCTGGCGGGTC
CGGAGGCTCTGGCGGCGCCTCAGGCGCAGGCTCCGGAGGCGGCGACAT
TCAGCTCACTCAACCCAACAGCGTGTCAACTTCTCTGGGATCCACCGT
GAAGCTGTCCTGTACTCTCAGCTCTGGGAATATCGAAAATAACTACGT
GCATTGGTACCAGCTCTATGAGGGGCGGAGCCCCACTACCATGATTTA
TGACGACGATAAACGCCCTGACGGTGTGCCTGATAGATTTTCTGGCAG
CATCGATCGGTCTAGCAATAGCGCATTCCTGACTATCCATAATGTGGC
AATCGAGGATGAGGCTATCTACTTCTGTCACTCCTATGTGAGCTCCTT
CAACGTCTTCGGTGGCGGCACAAAACTGACTGTTCTCGGGCCC
(SEQ ID NO: 358)
Panobacumab
GGCCCAGCCGGCCAGGCGCGAAGAACAGGTTGTTGAGTCAGGGGGCGG
ATTTGTGCAGCCTGGAGGATCTCTGAGACTCAGCTGCGCAGCCAGCGG
CTTCACCTTTTCACCATACTGGATGCACTGGGTGAGACAAGCTCCTGG
CAAGGGACTCGTCTGGGTGTCACGGATTAATTCTGACGGATCAACATA
CTACGCAGACTCAGTCAAAGGAAGGTTTACCATATCCAGAGATAACGC
TAGAAACACACTGTATCTGCAGATGAACTCACTCAGAGCTGAGGATAC
AGCAGTTTACTACTGTGCAAGAGACCGGTATTATGGTCCTGAGATGTG
GGGCCAGGGCACAATGGTGACCGTTAGCTCTGGCGGCGCAGGCTCTGG
GGCTGGATCAGGAAGCTCCGGTGCTGGTAGCGGCGATGTGGTGATGAC
CCAGTCTCCACTCAGCCTCCCCGTTACACTCGGGCAACCCGCCTCTAT
TTCTTGCCGCTCCTCCCAATCCCTCGTGTACTCTGACGGCAATACATA
CCTGAATTGGTTCCAGCAGAGACCTGGGCAGTCACCAAGGAGACTCAT
TTACAAGGTGAGCAATCGCGACAGCGGGGTGCCCGACCGGTTCAGCGG
CAGCGGCTCAGGGACCGATTTTACCCTCAAGATTTCAAGGGTGGAAGC
TGAAGATGTGGGAGTCTATTATTGTATGCAGGGCACCCACTGGCCCCT
GACATTTGGCGGCGGGACAAAGGTCGAGATCAAGGGGCCC
(SEQ ID NO: 359)
gantenerumab
GGCCCAGCCGGCCAGGCGCCAGGTCGAGCTGGTGGAGTCTGGCGGGGG
GCTGGTGCAACCTGGGGGAAGCCTGAGGCTGTCCTGCGCTGCATCAGG
GTTCACATTCTCTAGCTATGCAATGTCCTGGGTGAGGCAGGCCCCTGG
AAAAGGACTGGAGTGGGTCTCTGCAATCAATGCCTCTGGCACCCGCAC
TTATTATGCTGACAGCGTCAAGGGGAGGTTTACTATTTCTAGGGATAA
CTCTAAAAATACCCTGTACCTCCAGATGAACTCACTCAGGGCCGAGGA
TACTGCAGTTTACTATTGCGCTAGGGGTAAAGGTAACACCCACAAGCC
TTACGGATATGTGAGGTACTTCGACGTGTGGGGGCAGGGAACCGGTGG
CTCCGGCGGAAGCGGGGGAGCTTCCGGGGCTGGCTCTGGTGGGGGCGA
CATCGTGCTCACCCAGTCCCCAGCCACTCTGAGCCTGAGCCCTGGAGA
AAGAGCAACACTGTCTTGCCGGGCCTCCCAGTCCGTTTCCAGCAGCTA
CCTGGCCTGGTATCAGCAGAAACCAGGCCAGGCACCAAGGCTCCTGAT
CTATGGTGCCTCTTCCAGAGCAACCGGCGTGCCTGCTCGGTTCTCCGG
GTCCGGCTCAGGGACCGACTTCACACTGACTATATCCTCCCTGGAGCC
AGAGGACTTTGCCACATACTATTGTCTGCAAATCTACAATATGCCCAT
TACCTTTGGCCAGGGTACCAAAGTCGAGATCAAGGGGCCC
(SEQ ID NO: 360)
milatuzumab
GGCCCAGCCGGCCAGGCGCCAGGTCCAGCTGCAGCAGTCTGGATCCGA
GCTCAAAAAGCCCGGAGCCAGCGTTAAGGTTTCCTGCAAAGCCTCTGG
CTATACCTTCACTAATTACGGTGTGAACTGGATTAAGCAGGCCCCAGG
CCAGGGGCTCCAATGGATGGGCTGGATAAACCCTAATACTGGAGAGCC
TACTTTCGACGATGATTTCAAGGGGCGCTTCGCCTTCTCTCTGGATAC
CTCCGTGTCAACTGCCTACCTCCAGATCTCAAGCCTGAAAGCCGACGA
TACTGCCGTGTACTTCTGTTCTAGGTCCAGAGGGAAGAACGAGGCCTG
GTTCGCATACTGGGGTCAGGGGACACTGGTGACTGTGAGCTCTGGAGG
ATCAGCAGGGTCAGGGTCTTCCGGCGGGGCTAGCGGCTCAGGGGGCGA
CATTCAGCTCACCCAATCACCACTGTCTCTGCCCGTGACCCTCGGACA
GCCCGCTTCAATCTCATGCCGGTCTTCTCAGTCACTCGTCCATCGGAA
CGGCAACACTTATCTGCACTGGTTTCAACAGCGGCCAGGCCAATCTCC
CCGCCTGCTGATTTACACTGTGAGCAATCGGTTCTCAGGTGTTCCTGA
CAGATTTAGCGGGAGCGGTAGCGGCACTGATTTTACTCTGAAGATTTC
CCGCGTCGAAGCCGAGGACGTCGGGGTGTACTTTTGCAGCCAGAGCTC
TCATGTGCCCCCCACCTTCGGCGCAGGGACACGCCTGGAAATTAAGGG
GCCC (SEQ ID NO: 361)
veltuzumab
GGCCCAGCCGGCCAGGCGCCAGGTGCAGCTGCAGCAATCTGGCGCCGA
AGTGAAAAAACCAGGTTCCTCCGTCAAGGTGAGCTGCAAGGCCTCCGG
CTACACCTTTACCTCATACAACATGCACTGGGTGAAACAAGCTCCTGG
TCAGGGCCTGGAGTGGATTGGCGCAATCTATCCCGGGAATGGCGACAC
TTCTTATAACCAAAAGTTCAAAGGAAAGGCCACACTCACAGCCGACGA
AAGCACCAATACTGCCTACATGGAGCTGTCTAGCCTCCGCTCTGAGGA
TACTGCCTTCTACTACTGTGCTCGGTCCACTTACTACGGGGGGGATTG
GTACTTCGATGTGTGGGGGCAAGGCACTACTGTCACAGTTTCTTCTGG
GGGGGCCGGGAGCGGGGCCGGAAGCGGCAGCTCCGGCGCAGGCTCCGG
GGATATCCAGCTGACACAGAGCCCTTCATCACTCTCCGCCTCTGTTGG
AGATAGAGTCACAATGACTTGTAGGGCCTCCTCTTCCGTGTCATACAT
CCACTGGTTCCAGCAGAAGCCCGGTAAGGCTCCCAAGCCTTGGATTTA
TGCCACATCCAATCTGGCCTCAGGTGTGCCCGTCCGCTTCTCCGGTAG
CGGATCTGGGACTGATTATACTTTCACAATTAGCTCTCTGCAGCCAGA
AGATATTGCAACTTACTATTGCCAACAGTGGACATCCAATCCTCCTAC
TTTTGGAGGGGGGACTAAGCTCGAAATAAAGGGGCCC
(SEQ ID NO: 362)
Tanezumab
GGCCCAGCCGGCCAGGCGCCAGGTTCAGCTCCAAGAGTCAGGTCCTGG
GCTGGTTAAGCCTTCTGAGACACTGAGCCTGACCTGCACCGTTAGCGG
CTTCTCCCTGATCGGCTACGATCTGAACTGGATTCGGCAGCCACCCGG
AAAGGGCCTGGAATGGATTGGCATAATCTGGGGAGACGGGACAACTGA
CTATAATTCTGCCGTTAAGTCACGCGTGACCATATCTAAAGACACAAG
CAAGAACCAGTTCAGCCTGAAACTGTCCTCAGTCACAGCAGCAGATAC
TGCTGTGTATTACTGTGCCCGCGGGGGCTATTGGTACGCTACCTCATA
TTACTTTGATTACTGGGGGCAGGGCACCCTGGTGACCGTCTCCTCTGG
AGGCTCTGGTGGGTCTGGAGGAGCATCTGGGGCCGGGAGCGGCGGGGG
GGATATTCAGATGACTCAATCACCCTCAAGCCTCTCAGCCTCAGTCGG
GGACCGGGTGACAATCACCTGTAGGGCTTCACAAAGCATATCCAACAA
TCTGAATTGGTACCAGCAAAAACCAGGAAAAGCCCCAAAACTCCTGAT
ATACTATACCTCCCGGTTCCACAGCGGGGTGCCTAGCAGGTTCAGCGG
CTCCGGCAGCGGCACTGATTTCACTTTCACCATTTCCTCCCTGCAACC
AGAGGACATTGCAACTTATTATTGCCAGCAGGAGCATACCCTGCCATA
TACTTTCGGCCAGGGTACAAAGCTGGAGATAAAGGGGCCC
(SEQ ID NO: 363)
anrukinzumab
GGCCCAGCCGGCCAGGCGCGAAGTGCAACTGGTCGAAAGCGGGGGTGG
ACTGGTGCAGCCTGGGGGCAGCCTGCGCCTGAGCTGTGCAGCTTCAGG
CTTTACCTTCATCAGCTACGCTATGTCTTGGGTGAGACAGGCCCCCGG
AAAAGGACTCGAATGGGTGGCTAGCATCTCAAGCGGTGGCAATACATA
CTACCCCGACAGCGTCAAGGGCCGGTTTACCATCTCACGCGACAATGC
CAAGAATTCCCTGTACCTGCAGATGAACTCCCTGCGCGCTGAAGATAC
AGCCGTCTATTATTGCGCTCGGCTGGACGGCTACTACTTTGGCTTCGC
ATACTGGGGCCAGGGGACCCTGGTGACAGTCAGCTCCGGGGGGAGCGC
CGGCTCAGGGTCCTCCGGTGGTGCCTCTGGCTCAGGGGGGGACATTCA
AATGACACAGAGCCCCTCTTCTCTCTCAGCTAGCGTGGGCGACCGCGT
TACAATTACTTGCAAAGCCAGCGAATCCGTCGATAACTATGGGAAGTC
CCTGATGCACTGGTATCAACAGAAACCTGGAAAGGCTCCCAAACTGCT
CATCTACCGGGCTTCAAACCTGGAGAGCGGTGTGCCCTCACGGTTCTC
CGGATCTGGAAGCGGGACTGACTTTACCCTCACCATCTCCTCACTCCA
ACCAGAGGATTTCGCTACATATTATTGCCAGCAATCTAACGAGGATCC
ATGGACATTCGGGGGGGGCACAAAGGTTGAAATCAAGGGGCCC
(SEQ ID NO: 364)
ustekinumab
GGCCCAGCCGGCCAGGCGCGAGGTGCAACTCGTCCAGAGCGGCGCCGA
GGTTAAGAAGCCTGGCGAGTCCCTGAAAATTTCCTGCAAAGGCAGCGG
GTACTCTTTCACTACATACTGGCTGGGTTGGGTGCGGCAGATGCCCGG
GAAGGGGCTGGATTGGATCGGCATAATGTCCCCAGTGGATTCAGACAT
ACGCTATAGCCCCTCCTTCCAGGGTCAGGTGACCATGAGCGTCGATAA
GAGCATTACTACCGCCTACCTCCAGTGGAATTCCCTGAAGGCCTCTGA
TACAGCCATGTACTACTGCGCCCGCAGACGCCCAGGACAGGGATACTT
CGACTTCTGGGGCCAGGGAACCCTCGTGACCGTTTCAAGCGGCGGGGC
AGGGTCTGGCGCAGGAAGCGGCAGCAGCGGAGCCGGATCTGGGGATAT
TCAGATGACCCAGTCTCCTTCTTCCCTCTCTGCTAGCGTCGGCGATAG
GGTTACAATCACTTGCAGGGCCAGCCAGGGCATATCATCTTGGCTGGC
TTGGTATCAGCAGAAGCCAGAAAAGGCCCCTAAGAGCCTCATATATGC
TGCCAGCTCCCTGCAGTCCGGCGTGCCCTCCCGCTTCTCAGGCTCAGG
TTCAGGGACAGACTTCACACTGACAATCTCCTCCCTCCAGCCAGAGGA
TTTCGCCACCTATTATTGCCAACAGTACAATATCTACCCTTACACCTT
TGGCCAGGGCACCAAACTGGAAATCAAGGGGCCC
(SEQ ID NO: 365)
dacetuzumab
GGCCCAGCCGGCCAGGCGCGAAGTGCAACTGGTGGAGTCTGGGGGAGG
CCTGGTTCAGCCCGGTGGGAGCCTGCGGCTGTCCTGCGCCGCTTCCGG
CTACTCATTCACCGGATACTACATCCATTGGGTGAGGCAGGCCCCTGG
GAAGGGCCTGGAATGGGTGGCTAGAGTCATTCCTAATGCCGGTGGAAC
AAGCTACAATCAGAAATTCAAGGGGCGGTTTACCCTGAGCGTTGACAA
CTCTAAGAATACTGCATATCTGCAGATGAACTCTCTGCGGGCCGAGGA
CACCGCCGTGTATTACTGCGCCAGGGAAGGAATCTATTGGTGGGGCCA
AGGTACCCTGGTGACAGTCTCTTCCGGGGGCTCAGGAGGATCTGGAGG
TGCATCCGGCGCCGGAAGCGGAGGGGGCGACATCCAGATGACACAGTC
CCCTTCTTCTCTCTCTGCATCCGTTGGAGATAGAGTTACAATTACTTG
TCGGAGCTCTCAGTCACTGGTGCACAGCAACGGTAACACATTCCTGCA
CTGGTACCAGCAGAAACCTGGCAAAGCCCCTAAGCTGCTGATATACAC
AGTCTCCAACCGGTTCTCTGGAGTGCCCTCCAGGTTTTCAGGAAGCGG
GTCAGGGACAGACTTTACCCTGACTATCTCCTCTCTGCAACCTGAGGA
TTTCGCCACCTATTTCTGCAGCCAAACTACCCATGTTCCCTGGACTTT
TGGTCAGGGGACCAAGGTTGAGATCAAGGGGCCC
(SEQ ID NO: 366)
Alacizumab
GGCCCAGCCGGCCAGGCGCGAAGTCCAACTCGTGGAGTCCGGGGGAGG
CCTGGTGCAGCCCGGTGGGAGCCTGAGGCTCTCCTGTGCCGCCAGCGG
CTTCACATTCTCTTCCTACGGTATGTCATGGGTCAGGCAGGCCCCCGG
AAAAGGCCTGGAATGGGTCGCAACCATAACATCCGGCGGCAGCTATAC
ATACTACGTGGATAGCGTTAAGGGGAGGTTCACAATTTCCCGGGACAA
CGCCAAAAACACACTGTACCTGCAGATGAACTCTCTGCGGGCCGAGGA
TACCGCTGTGTACTATTGCGTGAGGATAGGCGAAGATGCTCTGGACTA
CTGGGGACAGGGGACTCTGGTCACAGTGTCAAGCGGCGGCAGCGCCGG
CTCAGGTAGCTCTGGGGGTGCCTCTGGATCCGGCGGCGATATCCAGAT
GACACAATCTCCTTCCAGCCTGTCCGCCTCCGTGGGTGACAGGGTGAC
CATTACATGTAGAGCATCACAGGACATCGCAGGGTCCCTGAATTGGCT
GCAACAAAAGCCTGGGAAAGCTATCAAAAGGCTGATTTACGCAACAAG
CTCTCTCGACAGCGGCGTTCCTAAGAGATTCTCTGGCTCTAGGTCAGG
AAGCGATTATACCCTGACTATCTCTAGCCTCCAGCCTGAAGATTTTGC
CACTTATTATTGCCTCCAGTACGGGTCTTTCCCACCTACCTTTGGTCA
GGGCACAAAAGTCGAGATAAAAGGGCCC
(SEQ ID NO: 367)
tigatuzumab
GGCCCAGCCGGCCAGGCGCGAAGTTCAGCTGGTGGAGTCCGGGGGGGG
TCTGGTCCAGCCAGGAGGTTCACTCCGCCTCTCTTGCGCAGCCTCAGG
CTTCACCTTTAGCTCTTACGTGATGTCCTGGGTCAGGCAGGCCCCTGG
CAAGGGTCTCGAATGGGTTGCCACAATCTCTTCAGGCGGAAGCTACAC
CTACTATCCCGACTCTGTTAAAGGAAGATTCACAATTTCCAGAGATAA
CGCCAAAAACACACTGTACCTGCAAATGAATTCACTGAGAGCTGAGGA
TACTGCTGTGTACTACTGCGCCAGACGCGGTGACTCCATGATCACCAC
CGACTATTGGGGTCAGGGGACTCTGGTCACCGTGTCATCCGGGGGAGC
CGGGAGCGGGGCTGGCAGCGGATCTTCTGGAGCAGGTTCTGGCGACAT
CCAGATGACACAAAGCCCTTCATCCCTCTCTGCATCTGTCGGCGATCG
CGTGACTATAACCTGCAAAGCCTCCCAGGACGTTGGAACTGCCGTTGC
TTGGTACCAGCAGAAACCCGGCAAGGCACCTAAGCTGCTGATCTACTG
GGCTAGCACAAGGCATACTGGGGTGCCCAGCCGCTTCTCCGGTTCCGG
CAGCGGTACAGATTTCACACTCACTATTAGCTCTCTGCAGCCTGAAGA
CTTCGCCACCTACTATTGCCAGCAGTACTCTAGCTACCGGACCTTCGG
ACAGGGAACAAAAGTGGAGATCAAGGGGCCC
(SEQ ID NO: 368)
Racotumomab
GGCCCAGCCGGCCAGGCGCCAGGTGCAGCTGCAGCAGTCCGGCGCCGA
GCTGGTGAAGCCAGGTGCATCTGTTAAGCTGTCCTGCAAGGCATCCGG
CTATACTTTCACCTCCTACGATATCAACTGGGTTCGGCAGAGGCCTGA
GCAAGGACTGGAGTGGATTGGGTGGATCTTCCCCGGAGATGGATCTAC
CAAGTATAACGAGAAGTTCAAGGGGAAAGCCACCCTGACCACAGATAA
AAGCTCAAGCACCGCCTATATGCAGCTCTCTCGGCTGACATCTGAAGA
TTCTGCCGTCTATTTTTGCGCTCGGGAGGACTACTACGACAACTCATA
TTATTTTGACTACTGGGGTCAGGGGACAACACTCACTGTCTCCAGCGG
CGGCTCAGGTGGGAGCGGCGGGGCTTCTGGTGCCGGATCCGGAGGCGG
TGATATCCAGATGACCCAGACAACTTCAAGCCTGTCCGCCTCACTGGG
GGATCGGGTCACCATTTCTTGCAGAGCCTCTCAGGATATCAGCAATTA
CCTGAATTGGTACCAGCAAAAACCCGATGGAACAGTGAAACTGCTGAT
CTACTACACATCTCGGCTGCATAGCGGAGTGCCCTCCAGGTTCAGCGG
CTCCGGGTCTGGCACAGACTACAGCCTGACCATCAGCAACCTGGAACA
GGAGGACATTGCCACCTATTTTTGTCAACAAGGAAATACCCTCCCTTG
GACATTTGGGGGAGGCACCAAGCTGGAAATTAAGGGGCCC
(SEQ ID NO: 369)
conatumumab
GGCCCAGCCGGCCAGGCGCCAGGTGCAACTCCAGGAATCCGGTCCCGG
CCTGGTGAAGCCATCTCAGACACTGTCCCTGACCTGCACAGTTTCCGG
CGGCAGCATCTCTAGCGGAGACTATTTCTGGTCCTGGATCAGACAGCT
CCCAGGCAAGGGCCTGGAGTGGATAGGGCATATTCATAACTCTGGAAC
AACCTACTATAATCCCTCTCTCAAATCACGGGTTACTATCTCCGTGGA
CACTTCCAAGAAACAGTTCTCCCTCAGACTGTCCTCAGTTACCGCAGC
CGACACCGCTGTGTATTACTGCGCAAGGGACAGGGGGGGCGACTATTA
CTACGGCATGGACGTGTGGGGCCAAGGTACAACTGTTACCGTTTCCTC
AGGTGGATCAGCCGGCAGCGGATCTTCTGGTGGCGCCTCCGGATCTGG
CGGAGAAATTGTGCTCACTCAATCCCCAGGGACACTGTCCCTCAGCCC
TGGCGAACGGGCCACTCTGTCCTGCAGGGCTAGCCAGGGCATTAGCCG
GAGCTACCTGGCCTGGTATCAGCAAAAGCCTGGGCAGGCCCCCTCTCT
GCTGATCTATGGTGCATCCTCCCGCGCCACCGGGATCCCTGACAGATT
TTCCGGATCCGGTAGCGGTACAGACTTCACTCTGACAATTTCCCGCCT
GGAGCCCGAGGATTTTGCTGTGTATTACTGCCAGCAATTTGGTTCTTC
ACCATGGACCTTTGGTCAAGGGACAAAGGTGGAAATAAAGGGGCCC
(SEQ ID NO: 370)
afutuzumab
GGCCCAGCCGGCCAGGCGCCAGGTCCAGCTGGTTCAAAGCGGAGCCGA
GGTTAAAAAACCTGGTTCTAGCGTGAAAGTGAGCTGCAAGGCCTCTGG
CTACGCATTCTCTTACAGCTGGATCAATTGGGTGCGCCAGGCCCCAGG
TCAGGGTCTGGAGTGGATGGGCAGGATCTTTCCAGGAGACGGAGATAC
CGATTACAACGGCAAGTTTAAAGGGAGGGTGACTATAACCGCTGACAA
GAGCACTTCAACAGCCTATATGGAACTCAGCTCTCTCAGAAGCGAGGA
TACAGCAGTCTACTATTGTGCTCGGAATGTCTTTGACGGGTACTGGCT
GGTGTACTGGGGCCAGGGAACCCTGGTCACAGTTAGCAGCGCAGGTGG
GGCCGGCTCTGGGGCAGGGAGCGGCTCCTCTGGCGCCGGCAGCGGGGA
CATAGTGATGACACAAACTCCTCTGTCTCTGCCAGTTACCCCCGGAGA
ACCCGCCAGCATTTCTTGTAGATCCTCTAAAAGCCTGCTGCATAGCAA
TGGGATCACCTACCTGTACTGGTATCTGCAGAAACCCGGCCAATCCCC
TCAGCTGCTGATTTACCAAATGTCCAACCTGGTGTCAGGAGTCCCAGA
TCGGTTCAGCGGATCCGGAAGCGGTACTGATTTTACCCTCAAAATATC
AAGGGTGGAAGCCGAGGACGTGGGCGTGTACTATTGCGCCCAGAATCT
GGAACTCCCTTATACATTCGGAGGCGGCACAAAAGTGGAAATAAAAGG
GCCC (SEQ ID NO: 380)
oportuzumab
GGCCCAGCCGGCCAGGCGCGAGGTGCAGCTGGTGCAAAGCGGGCCAGG
CCTCGTCCAGCCTGGGGGATCTGTTAGAATCTCATGTGCTGCCTCAGG
ATATACTTTTACAAACTATGGAATGAATTGGGTGAAGCAGGCACCTGG
GAAGGGCCTGGAGTGGATGGGTTGGATTAACACTTATACAGGCGAATC
AACATATGCCGACTCCTTTAAGGGCCGGTTCACCTTTTCTCTCGACAC
TTCCGCCAGCGCCGCCTACCTGCAAATCAACAGCCTGAGGGCCGAAGA
TACTGCCGTGTATTATTGCGCAAGATTTGCTATTAAGGGGGACTACTG
GGGTCAAGGGACCCTGCTGACAGTGTCCAGCGGCGGGAGCGGCGGTTC
CGGCGGAGCTTCCGGAGCCGGGTCCGGCGGAGGGGATATTCAGATGAC
CCAGTCACCCAGCAGCCTCTCTGCATCTGTGGGGGACAGGGTGACCAT
CACCTGTAGATCAACAAAATCTCTGCTGCATAGCAACGGAATCACTTA
CCTGTACTGGTATCAGCAGAAGCCTGGCAAAGCCCCAAAACTGCTGAT
CTATCAGATGTCCAATCTCGCATCTGGCGTCCCATCTAGGTTTAGCTC
CTCCGGCTCCGGTACAGACTTCACCCTGACCATATCAAGCCTGCAGCC
AGAGGACTTTGCCACTTACTATTGCGCTCAGAATCTCGAAATCCCTAG
GACATTTGGACAGGGCACAAAGGTCGAACTGAAAGGGCCC
(SEQ ID NO: 390)
citatuzumab
GGCCCAGCCGGCCAGGCGCGAGGTTCAACTCGTCCAATCTGGCCCTGG
GCTCGTCCAGCCCGGGGGATCCGTCCGCATCTCCTGCGCCGCCTCTGG
CTATACCTTCACTAATTATGGCATGAACTGGGTTAAACAGGCCCCAGG
CAAAGGTCTGGAGTGGATGGGCTGGATTAATACCTATACCGGCGAGTC
CACATACGCCGATAGCTTTAAGGGGAGGTTCACTTTCAGCCTCGATAC
CAGCGCTTCAGCAGCATACCTGCAGATTAACTCTCTGCGCGCCGAAGA
TACCGCTGTCTACTATTGCGCCCGGTTCGCTATTAAGGGGGATTACTG
GGGGCAGGGCACACTCCTGACCGTTTCAAGCGGCGGGTCCGCCGGCTC
CGGCTCATCTGGCGGGGCATCTGGGAGCGGAGGGGACATACAAATGAC
ACAGTCTCCAAGCTCTCTGAGCGCTTCTGTGGGGGATCGCGTCACCAT
TACATGCAGATCCACAAAATCCCTGCTGCATAGCAATGGCATTACTTA
TCTGTATTGGTACCAGCAGAAACCTGGCAAAGCTCCCAAACTGCTGAT
ATACCAGATGTCCAATCTGGCCTCCGGTGTTCCCAGCAGATTCTCAAG
CTCCGGCAGCGGGACAGACTTTACTCTGACCATCAGCAGCCTGCAGCC
CGAGGATTTCGCCACTTACTACTGCGCTCAGAACCTGGAAATCCCAAG
AACATTTGGCCAGGGCACTAAGGTTGAACTGAAGGGGCCC
(SEQ ID NO: 391)
siltuximab
GGCCCAGCCGGCCAGGCGCGAGGTGCAGCTGGTTGAGTCTGGTGGGAA
ACTGCTCAAGCCCGGAGGCTCACTGAAGCTGTCTTGTGCTGCTTCTGG
CTTTACCTTCAGCAGCTTCGCAATGTCTTGGTTTCGGCAAAGCCCAGA
GAAGCGCCTGGAGTGGGTTGCCGAGATATCTTCTGGAGGGTCATACAC
CTACTACCCCGACACTGTTACAGGTCGGTTCACCATCTCCAGGGATAA
TGCCAAGAATACCCTGTATCTGGAGATGTCTTCTCTCAGGTCAGAAGA
TACCGCTATGTACTATTGCGCTAGAGGTCTCTGGGGTTATTATGCACT
CGATTACTGGGGCCAGGGTACTAGCGTCACAGTGTCCTCTGGTGGGGC
CGGCTCTGGAGCCGGGAGCGGGTCAAGCGGAGCCGGATCTGGCCAGAT
TGTCCTCATCCAGTCCCCCGCCATCATGTCTGCTTCTCCAGGAGAGAA
GGTCACCATGACATGTTCCGCATCATCCTCCGTTTCTTACATGTATTG
GTATCAGCAGAAGCCAGGCTCTAGCCCACGCCTGCTGATCTATGACAC
TTCTAACCTCGCCTCCGGAGTGCCCGTGCGCTTTTCCGGCTCAGGCAG
CGGAACATCATATAGCCTGACCATAAGCCGCATGGAAGCCGAGGATGC
CGCAACCTATTATTGTCAACAGTGGTCAGGGTATCCCTACACATTCGG
GGGAGGCACCAAACTGGAAATTAAGGGGCCC
(SEQ ID NO: 392)
rafivirumab
GGCCCAGCCGGCCAGGCGCCAAGTGCAGCTGGTTCAGTCCGGGGCCGA
AGTCAAGAAGCCTGGGTCTAGCGTGAAGGTCTCTTGCAAAGCCAGCGG
GGGAACTTTCAACCGGTATACTGTTAACTGGGTGCGGCAAGCTCCTGG
CCAGGGACTGGAGTGGATGGGGGGAATCATCCCCATATTTGGAACCGC
TAACTATGCACAGCGCTTCCAGGGCAGACTGACTATAACCGCAGATGA
GTCCACCTCAACCGCCTACATGGAGCTGTCCTCTCTGCGGTCCGACGA
TACAGCCGTGTACTTTTGCGCCCGGGAGAACCTGGACAACTCTGGCAC
TTACTATTACTTCAGCGGCTGGTTCGACCCTTGGGGACAAGGCACCAG
CGTCACAGTCTCATCTGGCGGTTCTGGGGGGAGCGGCGGCGCTTCTGG
GGCCGGAAGCGGTGGCGGTCAGAGCGCACTGACCCAGCCTCGCAGCGT
CTCCGGCTCCCCTGGGCAGAGCGTGACAATATCTTGTACAGGCACCTC
CTCCGATATCGGGGGGTATAATTTCGTGTCATGGTACCAGCAACATCC
CGGCAAAGCCCCAAAGCTGATGATCTACGACGCCACTAAGAGGCCTTC
CGGGGTGCCCGATAGGTTCAGCGGGAGCAAATCTGGTAATACTGCCTC
ACTGACTATATCAGGCCTGCAGGCAGAAGACGAGGCAGATTATTACTG
CTGTTCTTACGCCGGTGACTACACACCTGGTGTGGTGTTTGGGGGCGG
CACCAAGCTGACTGTGCTGGGGCCC (SEQ ID NO: 393)
Foravirumab
GGCCCAGCCGGCCAGGCGCCAGGTCCAGCTGGTCGAGTCTGGCGGAGG
CGCCGTGCAGCCCGGGAGGTCCCTGAGACTGTCTTGCGCTGCTTCAGG
TTTCACTTTTTCTTCCTACGGCATGCACTGGGTCCGCCAAGCTCCTGG
AAAGGGACTGGAATGGGTCGCCGTCATACTGTACGACGGGAGCGACAA
GTTTTATGCCGATTCAGTGAAGGGTCGGTTTACTATTTCACGCGATAA
TTCCAAGAACACACTGTATCTGCAGATGAATTCCCTGCGGGCTGAAGA
TACAGCCGTGTACTACTGTGCAAAAGTGGCCGTGGCAGGGACTCACTT
TGACTATTGGGGCCAGGGGACTCTGGTGACTGTGTCCTCTGCAGGCGG
TTCCGCCGGCTCTGGCTCCAGCGGGGGCGCTTCAGGCTCCGGGGGCGA
TATCCAAATGACCCAAAGCCCATCCTCACTCTCCGCCTCTGTTGGCGA
TAGAGTCACTATTACCTGCAGGGCCTCTCAGGGGATCCGCAATGATCT
CGGATGGTACCAGCAGAAACCCGGAAAAGCTCCAAAACTGCTGATATA
CGCAGCTTCTTCTCTGCAGTCCGGGGTCCCCTCCCGGTTCTCCGGTAG
CGGTTCTGGAACCGACTTTACACTGACTATATCCTCTCTCCAGCCTGA
AGACTTCGCTACATATTACTGCCAGCAGCTGAACAGCTACCCTCCCAC
ATTCGGCGGCGGTACTAAGGTGGAAATCAAAGGGCCC
(SEQ ID NO: 394)
Farletuzumab
GGCCCAGCCGGCCAGGCGCGAAGTTCAGCTCGTGGAGTCTGGCGGAGG
CGTGGTCCAACCTGGCAGGTCCCTGAGGCTGTCTTGTTCTGCCAGCGG
ATTTACATTTTCCGGGTACGGACTGTCCTGGGTCAGACAGGCTCCAGG
GAAAGGCCTCGAATGGGTGGCAATGATCTCTAGCGGAGGCTCATACAC
CTATTACGCCGACTCCGTCAAGGGGCGCTTCGCCATCAGCAGAGATAA
TGCAAAGAATACTCTCTTCCTCCAGATGGATTCTCTCCGGCCCGAGGA
CACCGGTGTGTACTTCTGTGCTCGCCATGGGGATGACCCAGCCTGGTT
TGCTTACTGGGGCCAGGGAACTCCTGTGACCGTTTCTAGCGGGGGGGC
TGGCAGCGGGGCCGGTTCAGGTTCTTCCGGCGCCGGCTCCGGGGACAT
CCAGCTCACTCAGAGCCCATCTTCACTGTCAGCATCCGTCGGAGATAG
AGTGACTATAACCTGTTCAGTGTCCTCATCAATCAGCTCCAACAATCT
GCACTGGTACCAGCAGAAACCAGGAAAGGCACCAAAACCCTGGATATA
CGGCACCTCAAATCTGGCTTCCGGTGTGCCTTCCAGATTCTCAGGGAG
CGGATCCGGCACCGACTACACCTTTACAATCAGCTCCCTGCAGCCCGA
GGACATTGCAACATACTACTGTCAACAGTGGAGCTCCTATCCCTATAT
GTACACCTTCGGACAGGGAACAAAGGTTGAGATTAAAGGGCCC
(SEQ ID NO: 395)
Elotuzumab
GGCCCAGCCGGCCAGGCGCGAGGTGCAGCTCGTCGAGTCCGGAGGCGG
CCTGGTTCAGCCTGGCGGGTCTCTCCGCCTGTCCTGCGCCGCCTCCGG
ATTCGACTTTAGCAGATACTGGATGTCCTGGGTGAGACAGGCTCCTGG
AAAAGGACTCGAATGGATCGGGGAGATCAACCCCGATTCTTCCACCAT
CAACTACGCACCTAGCCTGAAAGATAAATTCATCATTTCCAGAGACAA
TGCCAAAAATTCACTGTACCTCCAAATGAACAGCCTGAGAGCTGAGGA
TACTGCTGTCTACTACTGCGCTAGGCCCGATGGGAATTACTGGTACTT
CGATGTGTGGGGGCAGGGCACTCTGGTTACCGTGTCATCAGGTGGCTC
CGGAGGGTCCGGCGGCGCAAGCGGAGCCGGATCCGGCGGAGGAGACAT
CCAGATGACACAGTCTCCATCCAGCCTCAGCGCCTCCGTTGGCGATCG
GGTGACAATCACCTGCAAGGCCTCACAGGACGTCGGAATCGCCGTTGC
TTGGTATCAACAAAAGCCCGGGAAGGTCCCCAAGCTGCTGATTTATTG
GGCCTCTACACGGCACACAGGTGTTCCAGATCGCTTCTCTGGTAGCGG
CTCCGGAACCGACTTTACTCTGACTATATCTTCTCTGCAGCCCGAGGA
TGTGGCCACTTACTACTGTCAGCAATATAGCTCCTACCCATACACTTT
TGGCCAGGGGACAAAAGTGGAGATCAAAGGGCCC
(SEQ ID NO: 396)
necitumumab
GGCCCAGCCGGCCAGGCGCCAGGTGCAGCTGCAAGAATCAGGGCCAGG
ACTCGTCAAACCCTCTCAAACACTGTCTCTGACTTGTACCGTGTCTGG
GGGCTCCATCTCATCCGGGGATTACTACTGGTCATGGATCAGGCAACC
ACCTGGCAAAGGTCTGGAGTGGATTGGCTATATCTACTACTCTGGGTC
AACCGATTATAACCCAAGCCTCAAGTCTCGGGTTACAATGAGCGTGGA
TACTAGCAAGAATCAATTCTCACTCAAGGTGAACTCTGTTACTGCCGC
TGACACCGCCGTGTACTATTGCGCTCGGGTCTCTATCTTCGGTGTGGG
GACCTTTGACTATTGGGGTCAAGGAACACTGGTCACTGTTTCAAGCGG
CGGCTCTGCAGGGTCAGGCTCATCCGGAGGCGCCTCCGGCTCTGGCGG
CGAAATAGTGATGACTCAGTCACCAGCTACTCTGTCCCTCTCCCCTGG
AGAGAGGGCTACACTCTCTTGCCGCGCCTCACAGTCTGTGAGCAGCTA
CCTCGCTTGGTACCAGCAGAAACCAGGTCAGGCCCCCCGGCTGCTGAT
CTATGACGCTAGCAATCGGGCTACTGGCATCCCCGCCAGATTTTCTGG
ATCTGGGTCAGGCACCGACTTCACACTGACTATAAGCTCACTGGAGCC
CGAAGACTTCGCCGTGTATTACTGCCATCAGTATGGAAGCACCCCCCT
GACCTTTGGGGGTGGTACCAAAGCCGAGATTAAGGGGCCC
(SEQ ID NO: 397)
figitumumab
GGCCCAGCCGGCCAGGCGCGAGGTTCAGCTCCTGGAGTCCGGGGGCGG
ACTGGTGCAGCCCGGGGGCTCACTGAGGCTGAGCTGCACAGCCTCTGG
CTTCACATTTAGCTCCTACGCCATGAATTGGGTGAGACAAGCCCCTGG
AAAGGGGCTGGAGTGGGTGTCTGCTATTTCAGGCTCAGGGGGGACAAC
CTTTTATGCCGACAGCGTGAAGGGCAGGTTCACCATTTCACGCGATAA
CTCACGCACTACCCTCTATCTGCAGATGAATTCCCTGCGGGCAGAAGA
CACAGCCGTCTATTATTGTGCAAAAGACCTGGGATGGTCTGACTCATA
TTATTATTATTATGGGATGGATGTTTGGGGGCAGGGGACCACCGTGAC
CGTCAGCAGCGGCGGGGCAGGATCTGGGGCCGGGTCTGGCTCATCAGG
GGCCGGTTCTGGGGATATACAGATGACCCAGTTCCCATCATCTCTCTC
AGCCTCTGTCGGGGATAGGGTTACCATTACTTGCAGAGCCAGCCAGGG
AATCAGAAATGATCTGGGCTGGTATCAACAGAAACCAGGTAAAGCCCC
CAAGAGGCTCATCTACGCCGCATCCCGCCTGCATCGGGGAGTCCCTTC
ACGCTTTTCCGGCTCTGGCTCAGGTACCGAGTTCACTCTCACTATTTC
CAGCCTCCAGCCAGAGGATTTTGCAACCTACTACTGCCTGCAACATAA
TTCTTATCCCTGTTCATTTGGTCAGGGCACAAAGCTCGAAATTAAGGG
GCCC (SEQ ID NO: 398)
Robatumumab
GGCCCAGCCGGCCAGGCGCGAAGTCCAACTGGTTCAGTCCGGGGGCGG
CCTGGTGAAACCCGGCGGCTCCCTGAGGCTCTCATGCGCCGCCAGCGG
ATTTACTTTTTCCTCATTTGCCATGCACTGGGTGAGGCAGGCACCAGG
AAAAGGACTGGAGTGGATCAGCGTCATTGATACAAGAGGTGCAACATA
TTACGCTGACAGCGTGAAGGGGAGATTTACAATTAGCCGCGATAACGC
CAAGAACTCCCTGTACCTGCAGATGAACTCCCTGCGGGCTGAAGACAC
AGCCGTGTACTATTGTGCAAGGCTGGGTAATTTTTATTACGGCATGGA
CGTTTGGGGGCAGGGGACTACTGTGACAGTTTCCTCAGGGGGGAGCGG
GGGGAGCGGGGGGGCTAGCGGCGCTGGCTCCGGAGGGGGAGAGATCGT
CCTGACACAGTCACCCGGGACTCTGTCTGTGAGCCCTGGCGAGAGAGC
AACTCTGTCATGCAGGGCCAGCCAAAGCATCGGCTCATCTCTGCACTG
GTACCAGCAGAAACCCGGTCAGGCCCCACGCCTGCTGATCAAATATGC
CAGCCAGAGCCTGTCAGGCATTCCTGACAGATTTTCTGGGAGCGGATC
AGGAACAGATTTCACACTCACAATATCCAGGCTGGAGCCCGAAGACTT
CGCTGTCTACTACTGCCACCAGTCCAGCAGACTCCCTCACACCTTCGG
GCAAGGGACAAAGGTCGAAATTAAAGGGCCC
(SEQ ID NO: 399)
vedolizumab
GGCCCAGCCGGCCAGGCGCCAGGTGCAGCTGGTCCAATCTGGTGCAGA
AGTGAAGAAACCTGGAGCTTCCGTGAAGGTGAGCTGTAAGGGGTCTGG
GTATACCTTTACAAGCTATTGGATGCATTGGGTGAGACAAGCCCCCGG
CCAGCGCCTCGAATGGATCGGGGAAATTGACCCTTCTGAATCTAACAC
TAACTACAATCAGAAATTTAAGGGGAGAGTGACCCTGACCGTGGACAT
TTCAGCTTCTACTGCCTACATGGAACTGTCCAGCCTGCGCTCTGAGGA
CACAGCCGTTTACTATTGTGCCCGGGGCGGGTACGACGGTTGGGACTA
TGCCATTGACTACTGGGGGCAAGGAACCCTGGTTACAGTCTCAAGCGG
TGGAAGCGCCGGTTCAGGTTCCTCAGGAGGGGCCTCAGGGTCAGGCGG
AGATGTCGTGATGACCCAATCTCCACTGAGCCTGCCTGTTACTCCCGG
CGAGCCCGCATCAATCAGCTGCAGATCCTCTCAATCCCTGGCTAAGAG
CTATGGAAATACCTACCTGTCATGGTACCTCCAGAAGCCTGGCCAATC
ACCCCAGCTGCTGATCTACGGCATTTCAAACAGATTCAGCGGCGTGCC
TGATCGCTTCTCCGGTTCAGGGTCTGGTACTGATTTCACACTGAAGAT
CTCTCGGGTGGAGGCAGAGGATGTGGGCGTCTACTACTGTCTCCAGGG
TACACACCAGCCATATACTTTCGGGCAAGGGACAAAGGTCGAGATCAA
GGGGCCC(SEQ ID NO: 400)

Table 12 depicts synthesized sequences.

TABLE 13

| Name | Sequence |
|---|---|
| mTFP1-BtsI-20-0 | ATATAGATGCCGTCCTAGCGAATCCTTGCGTCAATGGTTC |
| | GCAGTGTTTTTGCTTCAGTCAGATTCGCGGTACCATGGTG |
| | AGCAAGGGCGAGGAAACCACAATGGGCGTAATCAAGCCC |
| | GACATGAAGATCAAGCTGAAGATGGAGCACTGCCGTGTA |
| | AAATCCGAGAACCCTGGGCACAGGAAAGATACTT |
| | (SEQ ID NO: 401) |
| mTFP1-BtsI-20-1 | ATATAGATGCCGTCCTAGCGAATCCTTGCGTCAATGGTTC |
| | GCAGTGCATGAAGATCAAGCTGAAGATGGAGGGCAACGT |
| | GAATGGCCACGCCTTCGTGATCGAGGGCGAGGGCGAGG |
| | GCAAGCCCTACGACGGCACCAACACCACTGCCGTGTAAA |
| | ATCCGAGAACCCTGGGCACAGGAAAGATACTT |
| | (SEQ ID NO: 402) |
| mTFP1-BtsI-20-2 | ATATAGATGCCGTCCTAGCGAATCCTTGCGTCAATGGTTC |
| | GCAGTGGCCCTACGACGGCACCAACACCATCAACCTGGA |
| | GGTGAAGGAGGGAGCCCCCCTGCCCTTCTCCTACGACAT |
| | TCTGACCACCGCGTTCGCCTACACTGCCGTGTAAAATCCG |
| | AGAACCCTGGGCACAGGAAAGATACTT |
| | (SEQ ID NO: 403) |
| mTFP1-BtsI-20-3 | ATATAGATGCCGTCCTAGCGAATCCTTGCGTCAATGGTTC |
| | GCAGTGGACCACCGCGTTCGCCTACGGCAACAGGGCCTT |
| | CACCAAGTACCCCGACGACATCCCCAACTACTTCAAGCAG |
| | TCCTTCCCCGAGGGCTACTCTTCACTGCCGTGTAAAATCC |
| | GAGAACCCTGGGCACAGGAAAGATACTT |
| | (SEQ ID NO: 404) |
| mTFP1-BtsI-20-4 | ATATAGATGCCGTCCTAGCGAATCCTTGCGTCAATGGTTC |
| | GCAGTGCTTCCCCGAGGGCTACTCTTGGGAGCGCACCAT |
| | GACCTTCGAGGACAAGGGCATCGTGAAGGTGAAGTCCGA |
| | CATCTCCATGGAGGAGGACTCCTTCACTGCCGTGTAAAAT |
| | CCGAGAACCCTGGGCACAGGAAAGATACTT |
| | (SEQ ID NO: 405) |
| mTFP1-BtsI-20-5 | ATATAGATGCCGTCCTAGCGAATCCTTGCGTCAATGGTTC |
| | GCAGTGCTCCATGGAGGAGGACTCCTTCATCTACGAGATA |
| | CACCTCAAGGGCGAGAACTTCCCCCCCAACGGCCCCGTG |
| | ATGCAGAAAAAGACCACCGGCTGGGCACTGCCGTGTAAA |
| | ATCCGAGAACCCTGGGCACAGGAAAGATACTT |
| | (SEQ ID NO: 406) |
| mTFP1-BtsI-20-6 | ATATAGATGCCGTCCTAGCGAATCCTTGCGTCAATGGTTC |
| | GCAGTGGCAGAAAAAGACCACCGGCTGGGACGCCTCCAC |
| | CGAGAGGATGTACGTGCGCGACGGCGTGCTGAAGGGCG |
| | ACGTCAAGCACAAGCTGCTGCTGGAGGGCACTGCCGTGT |
| | AAAATCCGAGAACCCTGGGCACAGGAAAGATACTT |
| | (SEQ ID NO: 407) |
| mTFP1-BtsI-20-7 | ATATAGATGCCGTCCTAGCGAATCCTTGCGTCAATGGTTC |
| | GCAGTGGCACAAGCTGCTGCTGGAGGGCGGCGGCCACC |
| | ACCGCGTTGACTTCAAGACCATCTACAGGGCCAAGAAGG |
| | CGGTGAAGCTGCCCGACTATCACTTTGTCACTGCCGTGTA |
| | AAATCCGAGAACCCTGGGCACAGGAAAGATACTT |
| | (SEQ ID NO: 408) |
| mTFP1-BtsI-20-8 | ATATAGATGCCGTCCTAGCGAATCCTTGCGTCAATGGTTC |
| | GCAGTGAAGCTGCCCGACTATCACTTTGTGGACCACCGC |
| | ATCGAGATCCTGAACCACGACAAGGACTACAACAAGGTG |
| | ACCGTTTACGAGAGCGCCGTGGCCACTGCCGTGTAAAAT |
| | CCGAGAACCCTGGGCACAGGAAAGATACTT |
| | (SEQ ID NO: 409) |
| mTFP1-BtsI-20-9 | ATATAGATGCCGTCCTAGCGAATCCTTGCGTCAATGGTT |
| | CGCAGTGGTTTACGAGAGCGCCGTGGCCCGCAACTCCA |
| | CCGACGGCATGGACGAGCTGTACAAGTAAAAGCTTCCG |
| | GGATTCAGTGATTGAACTTCACTGCCGTGTAAAATCCGA |
| | GAACCCTGGGCACAGGAAAGATACTT |
| | (SEQ ID NO: 410) |
| mCitrine-BtsI-20-0 | ATATAGATGCCGTCCTAGCGTGTCGTGCCTCTTTATCTGT |
| | GCAGTGTTGTCGAGTCCTATGTAACCGTGGTACCATGGT |
| | GAGCAAGGGCGAGGAGCTGTTCACCGGGGTGGTGCCC |
| | ATCCTGGTCGAGCTGGACGGCGACACTGCCATTTCCGA |
| | TACACCGAAGCTGGGCACAGGAAAGATACTT |
| | (SEQ ID NO: 411) |
| mCitrine-BtsI-20-1 | ATATAGATGCCGTCCTAGCGTGTCGTGCCTCTTTATCTGT |
| | GCAGTGGGTCGAGCTGGACGGCGACGTAAACGGCCACA |
| | AGTTCAGCGTGTCCGGCGAGGGCGAGGGCGATGCCACC |
| | TACGGCAAGCTGACCCTGAAGTTCATCTGCCACTGCCAT |
| | TTCCGATACACCGAAGCTGGGCACAGGAAAGATACTT |
| | (SEQ ID NO: 412) |
| mCitrine-BtsI-20-2 | ATATAGATGCCGTCCTAGCGTGTCGTGCCTCTTTATCTGT |
| | GCAGTGAAGCTGACCCTGAAGTTCATCTGCACCACCGGC |
| | AAGCTGCCCGTGCCCTGGCCCACCCTCGTGACCACCTTC |
| | GGCTACGGCCTGATGTGCTTCGCCCACTGCCATTTCCGA |
| | TACACCGAAGCTGGGCACAGGAAAGATACTT |
| | (SEQ ID NO: 413) |
| mCitrine-BtsI-20-3 | ATATAGATGCCGTCCTAGCGTGTCGTGCCTCTTTATCTG |
| | TGCAGTGACGGCCTGATGTGCTTCGCCCGCTACCCCGAC |
| | CACATGAAGCAGCACGACTTCTTCAAGTCCGCCATGCCC |
| | GAAGGCTACGTCCAGGAGCGCACCCACTGCCATTTCCGA |
| | TACACCGAAGCTGGGCACAGGAAAGATACTT |
| | (SEQ ID NO: 414) |
| mCitrine-BtsI-20-4 | ATATAGATGCCGTCCTAGCGTGTCGTGCCTCTTTATCTGT |
| | GCAGTGCTACGTCCAGGAGCGCACCATCTTCTTCAAGGA |
| | CGACGGCAACTACAAGACCCGCGCCGAGGTGAAGTTCG |
| | AGGGCGACACCCTGGTGAACCGCATCGAGCACTGCCAT |
| | TTCCGATACACCGAAGCTGGGCACAGGAAAGATACTT |
| | (SEQ ID NO: 415) |
| mCitrine-BtsI-20-5 | ATATAGATGCCGTCCTAGCGTGTCGTGCCTCTTTATCTGT |
| | GCAGTGACCCTGGTGAACCGCATCGAGCTGAAGGGCATC |
| | GACTTCAAGGAGGACGGCAACATCCTGGGGCACAAGCT |
| | GGAGTACAACTACAACAGCCACAACGTCTCACTGCCATT |
| | TCCGATACACCGAAGCTGGGCACAGGAAAGATACTT |
| | (SEQ ID NO: 416) |
| mCitrine-BtsI-20-6 | ATATAGATGCCGTCCTAGCGTGTCGTGCCTCTTTATCTGT |
| | GCAGTGACAACTACAACAGCCACAACGTCTATATCATGG |
| | CCGACAAGCAGAAGAACGGCATCAAGGTGAACTTCAAGA |
| | TCCGCCACAACATCGAGGACGGCAGCACTGCCATTTCC |
| | GATACACCGAAGCTGGGCACAGGAAAGATACTT |
| | (SEQ ID NO: 417) |
| mCitrine-BtsI-20-7 | ATATAGATGCCGTCCTAGCGTGTCGTGCCTCTTTATCTGT |
| | GCAGTGCCACAACATCGAGGACGGCAGCGTGCAGCTCG |
| | CCGACCACTACCAGCAGAACACCCCCATCGGCGACGGC |
| | CCCGTGCTGCTGCCCGACAACCACTACCTGCACTGCCA |
| | TTTCCGATACACCGAAGCTGGGCACAGGAAAGATACTT |
| | (SEQ ID NO: 418) |
| mCitrine-BtsI-20-8 | ATATAGATGCCGTCCTAGCGTGTCGTGCCTCTTTATCTG |
| | TGCAGTGCTGCCCGACAACCACTACCTGAGCTACCAGTC |
| | CAAACTGAGCAAAGACCCCAACGAGAAGCGCGATCACA |
| | TGGTCCTGCTGGAGTTCGTGACCGCCGCACTGCCATTT |
| | CCGATACACCGAAGCTGGGCACAGGAAAGATACTT |
| | (SEQ ID NO: 419) |
| mCitrine-BtsI-20-9 | ATATAGATGCCGTCCTAGCGTGTCGTGCCTCTTTATCTG |
| | TGCAGTGTGCTGGAGTTCGTGACCGCCGCCGGGATCA |
| | CTCTCGGCATGGACGAGCTGTACAAGTAAAAGCTTTGA |
| | AGATATGACGACCCCTGTTCACTGCCATTTCCGATACAC |
| | CGAAGCTGGGCACAGGAAAGATACTT (SEQ ID NO: 420) |
| mApple-BtsI-20-0 | ATATAGATGCCGTCCTAGCGATTTAAACGGTGAGGTGT |
| | GCGCAGTGTTGTAAGATGGAAGCCGGGATAGGTACCA |
| | TGGTGAGCAAGGGCGAGGAGAATAACATGGCCATCAT |
| | CAAGGAGTTCATGCGCTTCAAGGTGCACATGGACACT |
| | GCTGATAGCCAGCGAAACGATATGGGCACAGGAAAG |
| | ATACTT (SEQ ID NO: 421) |
| mApple-BtsI-20-1 | ATATAGATGCCGTCCTAGCGATTTAAACGGTGAGGTGT |
| | GCGCAGTGTGCGCTTCAAGGTGCACATGGAGGGCTCC |
| | GTGAACGGCCACGAGTTCGAGATCGAGGGCGAGGGC |
| | GAGGGCCGCCCCTACGAGGCCTTTCAGACCGCCACTG |
| | CTGATAGCCAGCGAAACGATATGGGCACAGGAAAGAT |
| | ACTT (SEQ ID NO: 422) |
| mApple-BtsI-20-2 | ATATAGATGCCGTCCTAGCGATTTAAACGGTGAGGTGT |
| | GCGCAGTGCCTACGAGGCCTTTCAGACCGCTAAGCTG |
| | AAGGTGACCAAGGGTGGCCCCCTGCCCTTCGCCTGGG |
| | ACATCCTGTCCCCTCAGTTCATGTACGGCTCCACACTG |
| | CTGATAGCCAGCGAAACGATATGGGCACAGGAAAGAT |
| | ACTT (SEQ ID NO: 423) |
| mApple-BtsI-20-3 | ATATAGATGCCGTCCTAGCGATTTAAACGGTGAGGTGT |
| | GCGCAGTGCCCCTCAGTTCATGTACGGCTCCAAGGTCT |
| | ACATTAAGCACCCAGCCGACATCCCCGACTACTTCAAG |
| | CTGTCCTTCCCCGAGGGCTTCAGGTGGGAGCCACTGCT |
| | GATAGCCAGCGAAACGATATGGGCACAGGAAAGATAC |
| | TT (SEQ ID NO: 424) |
| mApple-BtsI-20-4 | ATATAGATGCCGTCCTAGCGATTTAAACGGTGAGGTGT |
| | GCGCAGTGCCGAGGGCTTCAGGTGGGAGCGCGTGATG |
| | AACTTCGAGGACGGCGGCATTATTCACGTTAACCAGGA |
| | CTCCTCCCTGCAGGACGGCGTGTTCATCTACACACTGC |
| | TGATAGCCAGCGAAACGATATGGGCACAGGAAAGATA |
| | CTT (SEQ ID NO: 425) |
| mApple-BtsI-20-5 | ATATAGATGCCGTCCTAGCGATTTAAACGGTGAGGTGT |
| | GCGCAGTGCAGGACGGCGTGTTCATCTACAAGGTGAA |
| | GCTGCGCGGCACCAACTTCCCCTCCGACGGCCCCGTAA |
| | TGCAGAAAAAGACCATGGGCTGGGAGGCCACTGCTGA |
| | TAGCCAGCGAAACGATATGGGCACAGGAAAGATACTT |
| | (SEQ ID NO: 426) |
| mApple-BtsI-20-6 | ATATAGATGCCGTCCTAGCGATTTAAACGGTGAGGTGT |
| | GCGCAGTGAAGACCATGGGCTGGGAGGCCTCCGAGG |
| | AGCGGATGTACCCCGAGGACGGCGCCTTAAAGAGCGA |
| | GATCAAAAAGAGGCTGAAGCTGAAGGACGGCGCACTG |
| | CTGATAGCCAGCGAAACGATATGGGCACAGGAAAGAT |
| | ACTT (SEQ ID NO: 427) |
| mApple-BtsI-20-7 | ATATAGATGCCGTCCTAGCGATTTAAACGGTGAGGTGT |
| | GCGCAGTGAGGCTGAAGCTGAAGGACGGCGGCCACTA |
| | CGCCGCCGAGGTCAAGACCACCTACAAGGCCAAGAAG |
| | CCCGTGCAGCTGCCCGGCGCCTACATCGTCGACCACT |
| | GCTGATAGCCAGCGAAACGATATGGGCACAGGAAAG |
| | ATACTT (SEQ ID NO: 428) |
| mApple-BtsI-20-8 | ATATAGATGCCGTCCTAGCGATTTAAACGGTGAGGTGT |
| | GCGCAGTGCCGGCGCCTACATCGTCGACATCAAGTTG |
| | GACATCGTGTCCCACAACGAGGACTACACCATCGTGG |
| | AACAGTACGAACGCGCCGAGGGCCGCCACTCCACCAC |
| | TGCTGATAGCCAGCGAAACGATATGGGCACAGGAAAG |
| | ATACTT (SEQ ID NO: 429) |
| mApple-BtsI-20-9 | ATATAGATGCCGTCCTAGCGATTTAAACGGTGAGGTGT |
| | GCGCAGTGCGAGGGCCGCCACTCCACCGGCGGCATGG |
| | ACGAGCTGTACAAGTAAAAGCTTTTCCACAGCTCTATGA |
| | GGTGTTCACTGCTGATAGCCAGCGAAACGATATGGGC |
| | ACAGGAAAGATACTT (SEQ ID NO: 430) |
| mut3-BspQI-20-0 | ATATAGATGCCGTCCTAGCGCATCCGATGGTGGTGTAG |
| | ATGCTCTTCTTTTGGTGTCGCAACATGATCTACGGTACC |
| | ATGGTGAGCAAGGGCGAGGAGAATAACATGGCCATCA |
| | TCAAGGAGTTCATGCGCTTCAAGGTGCAGAAGAGCGGA |
| | GAACGGTCAACTATCCATGGGCACAGGAAAGATACTT |
| | (SEQ ID NO: 431) |
| mut3-BspQI-20-1 | ATATAGATGCCGTCCTAGCGCATCCGATGGTGGTGTAG |
| | ATGCTCTTCCAAGGAGTTCATGCGCTTCAAGGTGCACA |
| | TGGAGGGCTCCGTGAACGGCCACGAGTTCGAGATCGA |
| | GGGCGAGGGCGAGGGCCGCCCCTACGAGGGAAGAGC |
| | GGAGAACGGTCAACTATCCATGGGCACAGGAAAGATA |
| | CTT (SEQ ID NO: 432) |
| mut3-BspQI-20-2 | ATATAGATGCCGTCCTAGCGCATCCGATGGTGGTGTAG |
| | ATGCTCTTCGGCGAGGGCCGCCCCTACGAGGCCTTTCA |
| | GACCGCTAAGCTGAAGGTGACCAAGGGTGGCCCCCTG |
| | CCCTTCGCCTGGGACATCCTGTCCCCGAAGAGCGGAG |
| | AACGGTCAACTATCCATGGGCACAGGAAAGATACTT |
| | (SEQ ID NO: 433) |
| mut3-BspQI-20-3 | ATATAGATGCCGTCCTAGCGCATCCGATGGTGGTGTAG |
| | ATGCTCTTCCTTCGCCTGGGACATCCTGTCCCCTCAGTT |
| | CATGTACGGCTCCAAGGTCTACATTAAGCACCCAGCCG |
| | ACATCCCCGACTACTTCAAGCTGTCCTTGAAGAGCGG |
| | AGAACGGTCAACTATCCATGGGCACAGGAAAGATACTT |
| | (SEQ ID NO: 434) |
| mut3-BspQI-20-4 | ATATAGATGCCGTCCTAGCGCATCCGATGGTGGTGTAG |
| | ATGCTCTTCCATCCCCGACTACTTCAAGCTGTCCTTCCC |
| | CGAGGGCTTCAGGTGGGAGCGCGTGATGAACTTCGAG |
| | GACGGCGGCATTATTCACGTTAACCAGGAGAAGAGCG |
| | GAGAACGGTCAACTATCCATGGGCACAGGAAAGATAC |
| | TT (SEQ ID NO: 435) |
| mut3-BspQI-20-5 | ATATAGATGCCGTCCTAGCGCATCCGATGGTGGTGTAG |
| | ATGCTCTTCACGGCGGCATTATTCACGTTAACCAGGACT |
| | CCTCCCTGCAGGACGGCGTGTTCATCTACAAGGTGAAG |
| | CTGCGCGGCACCAACTTCCCCTCCGACGGAAGAGCGGA |
| | GAACGGTCAACTATCCATGGGCACAGGAAAGATACTT |
| | (SEQ ID NO: 436) |
| mut3-BspQI-20-6 | ATATAGATGCCGTCCTAGCGCATCCGATGGTGGTGTAG |
| | ATGCTCTTCCGCGGCACCAACTTCCCCTCCGACGGCCCC |
| | GTAATGCAGAAAAAGACCATGGGCTGGGAGGCCTCCGA |
| | GGAGCGGATGTACCCCGAGGACGGCGAAGAGCGGAGA |
| | ACGGTCAACTATCCATGGGCACAGGAAAGATACTT |
| | (SEQ ID NO: 437) |
| mut3-BspQI-20-7 | ATATAGATGCCGTCCTAGCGCATCCGATGGTGGTGTAG |
| | ATGCTCTTCGGAGCGGATGTACCCCGAGGACGGCGCCT |
| | TAAAGAGCGAGATCAAAAAGAGGCTGAAGCTGAAGGAC |
| | GGCGGCCACTACGCCGCCGAGGTGAAGAGCGGAGAAC |
| | GGTCAACTATCCATGGGCACAGGAAAGATACTT |
| | (SEQ ID NO: 438) |
| mut3-BspQI-20-8 | ATATAGATGCCGTCCTAGCGCATCCGATGGTGGTGTAG |
| | ATGCTCTTCGCGGCCACTACGCCGCCGAGGTCAAGACC |
| | ACCTACAAGGCCAAGAAGCCCGTGCAGCTGCCCGGCGC |
| | CTACATCGTCGACATCAAGTTGGACATCGGAAGAGCGG |
| | AGAACGGTCAACTATCCATGGGCACAGGAAAGATACTT |
| | (SEQ ID NO: 439) |
| mut3-BspQI-20-9 | ATATAGATGCCGTCCTAGCGCATCCGATGGTGGTGTAG |
| | ATGCTCTTCTACATCGTCGACATCAAGTTGGACATCGTG |
| | TCCCACAACGAGGACTACACCATCGTGGAACAGTACGA |
| | ACGCGCCGAGGGCCGCCACTCCACCGGCGAAGAGCGG |
| | AGAACGGTCAACTATCCATGGGCACAGGAAAGATACTT |
| | (SEQ ID NO: 440) |
| mut3-BspQI-20-10 | ATATAGATGCCGTCCTAGCGCATCCGATGGTGGTGTAG |
| | ATGCTCTTCCGAGGGCCGCCACTCCACCGGCGGCATGG |
| | ACGAGCTGTACAAGTAAAAGCTTGCAAACATGACTAGG |
| | AACCGTTTTGAAGAGCGGAGAACGGTCAACTATCCATG |
| | GGCACAGGAAAGATACTT (SEQ ID NO: 441) |
| trastuzumab-BtsI-20-0 | CCCTTTAATCAGATGCGTCGCTTAAACCGGCCAACATAC |
| | CGCAGTGTTGCTTATTCGTGCCGTGTTATGGCCCAGCCG |
| | GCCAGGCGCGAAGTGCAGCTGGTGGAGTCAGGCGGTG |
| | GACTGGTGCAGCCAGGAGGTTCCCTGCACTGCTCGAAA |
| | GGAACGAGTAGCATGGTCGCCCTTATTACTACCA |
| | (SEQ ID NO: 442) |
| trastuzumab-BtsI-20-1 | CCCTTTAATCAGATGCGTCGCTTAAACCGGCCAACATAC |
| | CGCAGTGTGCAGCCAGGAGGTTCCCTGAGACTCTCATG |
| | CGCAGCAAGCGGTTTTAATATCAAGGACACTTATATACA |
| | CTGGGTGCGCCAAGCCCCCGGAAAGCACTGCTCGAAAG |
| | GAACGAGTAGCATGGTCGCCCTTATTACTACCA |
| | (SEQ ID NO: 443) |
| trastuzumab-BtsI-20-2 | CCCTTTAATCAGATGCGTCGCTTAAACCGGCCAACATAC |
| | CGCAGTGCGCCAAGCCCCCGGAAAGGGTCTGGAGTGG |
| | GTGGCCAGAATATACCCCACAAACGGCTATACCAGGTA |
| | CGCAGATTCAGTGAAGGGGAGATTCACCACTGCTCGAA |
| | AGGAACGAGTAGCATGGTCGCCCTTATTACTACCA |
| | (SEQ ID NO: 444) |
| trastuzumab-BtsI-20-3 | CCCTTTAATCAGATGCGTCGCTTAAACCGGCCAACATAC |
| | CGCAGTGAGATTCAGTGAAGGGGAGATTCACCATAAGC |
| | GCTGACACATCTAAGAATACTGCTTACCTGCAAATGAAT |
| | TCCCTGAGGGCAGAGGATACAGCTGCACTGCTCGAAAG |
| | GAACGAGTAGCATGGTCGCCCTTATTACTACCA |
| | (SEQ ID NO: 445) |
| trastuzumab-BtsI-20-4 | CCCTTTAATCAGATGCGTCGCTTAAACCGGCCAACATAC |
| | CGCAGTGCTGAGGGCAGAGGATACAGCTGTTTATTACT |
| | GCAGCCGGTGGGGCGGAGATGGCTTTTACGCCATGGAC |
| | TATTGGGGGCAGGGAACCCTGGTCACCCACTGCTCGAA |
| | AGGAACGAGTAGCATGGTCGCCCTTATTACTACCA |
| | (SEQ ID NO: 446) |
| trastuzumab-BtsI-20-5 | CCCTTTAATCAGATGCGTCGCTTAAACCGGCCAACATAC |
| | CGCAGTGGGCAGGGAACCCTGGTCACCGTTTCCAGCG |
| | GTGGGTCAGGGGGCAGCGGCGGCGCCAGCGGAGCAG |
| | GGAGCGGTGGAGGCGATATCCAAATGACACACTGCTCG |
| | AAAGGAACGAGTAGCATGGTCGCCCTTATTACTACCA |
| | (SEQ ID NO: 447) |
| trastuzumab-BtsI-20-6 | CCCTTTAATCAGATGCGTCGCTTAAACCGGCCAACATA |
| | CCGCAGTGGGTGGAGGCGATATCCAAATGACACAGTC |
| | CCCCTCTAGCCTGAGCGCCAGCGTCGGTGACAGGGTG |
| | ACCATTACATGCAGGGCCTCTCAGGACACTGCTCGAAA |
| | GGAACGAGTAGCATGGTCGCCCTTATTACTACCA |
| | (SEQ ID NO: 448) |
| trastuzumab-BtsI-20-7 | CCCTTTAATCAGATGCGTCGCTTAAACCGGCCAACATA |
| | CCGCAGTGTACATGCAGGGCCTCTCAGGATGTTAATAC |
| | TGCCGTTGCATGGTACCAGCAGAAGCCCGGGAAGGCA |
| | CCAAAGCTGCTGATCTATTCCGCTTCCTCACTGCTCGA |
| | AAGGAACGAGTAGCATGGTCGCCCTTATTACTACCA |
| | (SEQ ID NO: 449) |
| trastuzumab-BtsI-20-8 | CCCTTTAATCAGATGCGTCGCTTAAACCGGCCAACAT |
| | ACCGCAGTGAGCTGCTGATCTATTCCGCTTCCTTTCT |
| | GTACAGCGGAGTGCCTAGCAGGTTTTCCGGATCTCG |
| | CAGCGGAACTGATTTTACACTCACCATCAGCAGCACT |
| | GCTCGAAAGGAACGAGTAGCATGGTCGCCCTTATTA |
| | CTACCA (SEQ ID NO: 450) |
| trastuzumab-BtsI-20-9 | CCCTTTAATCAGATGCGTCGCTTAAACCGGCCAACAT |
| | ACCGCAGTGACTGATTTTACACTCACCATCAGCAGCC |
| | TCCAACCTGAGGATTTTGCCACCTATTATTGCCAGCA |
| | ACACTACACCACTCCACCCACTTTCGGCCACTGCTC |
| | GAAAGGAACGAGTAGCATGGTCGCCCTTATTACTAC |
| | CA (SEQ ID NO: 451) |
| trastuzumab-BtsI-20-10 | CCCTTTAATCAGATGCGTCGCTTAAACCGGCCAACA |
| | TACCGCAGTGCACCACTCCACCCACTTTCGGCCAG |
| | GGAACTAAGGTGGAAATAAAAGGGCCCGGGCACA |
| | GCAATCAAAAGTATTCACTGCTCGAAAGGAACGAG |
| | TAGCATGGTCGCCCTTATTACTACCA (SEQ ID NO: 452) |
| Cetuximab-BtsI-20-0 | CCCTTTAATCAGATGCGTCGTGCTCTTTATTCGTTG |
| | CGTCGCAGTGTTTTTGCTTCAGTCAGATTCGCGGC |
| | CCAGCCGGCCAGGCGCCAGGTTCAGCTCAAGCAG |
| | TCTGGACCCGGACTGGTGCAGCCCTCTCAGTCTCT |
| | CCACTGCAGAACGAAGCACCGATAAGAGGTCGCC |
| | CTTATTACTACCA (SEQ ID NO: 453) |
| Cetuximab-BtsI-20-1 | CCCTTTAATCAGATGCGTCGTGCTCTTTATTCGTTG |
| | CGTCGCAGTGGTGCAGCCCTCTCAGTCTCTCTCTA |
| | TCACCTGCACAGTGTCTGGTTTCTCTCTCACCAAC |
| | TACGGGGTCCATTGGGTTCGGCAGTCCCCAGGGA |
| | ACACTGCAGAACGAAGCACCGATAAGAGGTCGCC |
| | CTTATTACTACCA (SEQ ID NO: 454) |
| Cetuximab-BtsI-20-2 | CCCTTTAATCAGATGCGTCGTGCTCTTTATTCGTT |
| | GCGTCGCAGTGTCGGCAGTCCCCAGGGAAAGGG |
| | CTCGAATGGCTGGGCGTGATCTGGTCCGGCGGCA |
| | ATACCGACTACAACACCCCATTTACTTCCAGGCTG |
| | TCAACACTGCAGAACGAAGCACCGATAAGAGGTC |
| | GCCCTTATTACTACCA (SEQ ID NO: 455) |
| Cetuximab-BtsI-20-3 | CCCTTTAATCAGATGCGTCGTGCTCTTTATTCGTT |
| | GCGTCGCAGTGCCCCATTTACTTCCAGGCTGTCA |
| | ATTAATAAGGACAATTCTAAGAGCCAGGTCTTCTT |
| | TAAGATGAACTCTCTCCAGTCTAATGATACTGCCA |
| | TCCACTGCAGAACGAAGCACCGATAAGAGGTCG |
| | CCCTTATTACTACCA (SEQ ID NO: 456) |
| Cetuximab-BtsI-20-4 | CCCTTTAATCAGATGCGTCGTGCTCTTTATTCGTT |
| | GCGTCGCAGTGTCTCCAGTCTAATGATACTGCCA |
| | TCTACTACTGTGCCCGGGCACTCACATACTACGA |
| | TTATGAATTCGCTTACTGGGGCCAGGGCACCCTC |
| | GTCACACTGCAGAACGAAGCACCGATAAGAGGTC |
| | GCCCTTATTACTACCA (SEQ ID NO: 457) |
| Cetuximab-BtsI-20-5 | CCCTTTAATCAGATGCGTCGTGCTCTTTATTCGTT |
| | GCGTCGCAGTGGGCCAGGGCACCCTCGTCACCG |
| | TGAGCGCAGGAGGATCTGCTGGCTCTGGGTCAA |
| | GCGGTGGCGCTTCCGGCTCAGGGGGAGACATCC |
| | TGCTCACTGCAGAACGAAGCACCGATAAGAGGT |
| | CGCCCTTATTACTACCA (SEQ ID NO: 458) |
| Cetuximab-BtsI-20-6 | CCCTTTAATCAGATGCGTCGTGCTCTTTATTCGTT |
| | GCGTCGCAGTGGCTCAGGGGGAGACATCCTGCT |
| | CACCCAGAGCCCCGTGATTCTGTCCGTTAGCCCC |
| | GGAGAACGCGTTTCTTTTAGCTGTCGCGCATCTC |
| | AGAGCCACTGCAGAACGAAGCACCGATAAGAGG |
| | TCGCCCTTATTACTACCA (SEQ ID NO: 459) |
| Cetuximab-BtsI-20-7 | CCCTTTAATCAGATGCGTCGTGCTCTTTATTCGTT |
| | GCGTCGCAGTGAGCTGTCGCGCATCTCAGAGCA |
| | TCGGTACCAACATTCACTGGTATCAGCAGCGGAC |
| | CGACGGGAGCCCTCGCCTCCTGATAAAATATGCT |
| | TCTGACACTGCAGAACGAAGCACCGATAAGAGGT |
| | CGCCCTTATTACTACCA (SEQ ID NO: 460) |
| Cetuximab-BtsI-20-8 | CCCTTTAATCAGATGCGTCGTGCTCTTTATTCGTT |
| | GCGTCGCAGTGTCGCCTCCTGATAAAATATGCTT |
| | CTGAGTCAATTAGCGGTATCCCCTCCAGATTTAG |
| | CGGGAGCGGTTCTGGGACCGATTTCACACTGAG |
| | CATCACACTGCAGAACGAAGCACCGATAAGAGG |
| | TCGCCCTTATTACTACCA (SEQ ID NO: 461) |
| Cetuximab-BtsI-20-9 | CCCTTTAATCAGATGCGTCGTGCTCTTTATTCGTT |
| | GCGTCGCAGTGGGACCGATTTCACACTGAGCATC |
| | AACTCTGTGGAGTCTGAAGATATCGCTGATTATTA |
| | CTGTCAGCAAAACAACAATTGGCCTACCACCTTCG |
| | GCACTGCAGAACGAAGCACCGATAAGAGGTCGC |
| | CCTTATTACTACCA (SEQ ID NO: 462) |
| Cetuximab-BtsI-20-10 | CCCTTTAATCAGATGCGTCGTGCTCTTTATTCGTT |
| | GCGTCGCAGTGAACAATTGGCCTACCACCTTCGG |
| | CGCCGGCACCAAGCTGGAACTGAAAGGGCCCCC |
| | GGGATTCAGTGATTGAACTTCACTGCAGAACGAA |
| | GCACCGATAAGAGGTCGCCCTTATTACTACCA |
| | (SEQ ID NO: 463) |
| alemtuzumab-BtsI-20-0 | CCCTTTAATCAGATGCGTCGTGAGCCTTATGATT |
| | TCCCGTGCAGTGTTGTCGAGTCCTATGTAACCGT |
| | GGCCCAGCCGGCCAGGCGCCAAGTTCAGCTCCA |
| | GGAGTCAGGTCCTGGTCTGGTGAGACCATCCCA |
| | GACCCCACTGCGCTCATTCAGGAAAACGGACGG |
| | TCGCCCTTATTACTACCA (SEQ ID NO: 464) |
| alemtuzumab-BtsI-20-1 | CCCTTTAATCAGATGCGTCGTGAGCCTTATGATTT |
| | CCCGTGCAGTGCTGGTGAGACCATCCCAGACCCT |
| | CTCTCTCACTTGTACCGTTTCCGGCTTCACATTCA |
| | CCGATTTCTATATGAACTGGGTTAGGCAACCACCA |
| | CACTGCGCTCATTCAGGAAAACGGACGGTCGCCC |
| | TTATTACTACCA (SEQ ID NO: 465) |
| alemtuzumab-BtsI-20-2 | CCCTTTAATCAGATGCGTCGTGAGCCTTATGATTT |
| | CCCGTGCAGTGGAACTGGGTTAGGCAACCACCAG |
| | GCCGGGGGCTGGAATGGATCGGTTTTATCAGAGA |
| | TAAAGCCAAGGGATATACTACTGAGTACAACCCC |
| | TCTGCACTGCGCTCATTCAGGAAAACGGACGGTC |
| | GCCCTTATTACTACCA (SEQ ID NO: 466) |
| alemtuzumab-BtsI-20-3 | CCCTTTAATCAGATGCGTCGTGAGCCTTATGATTT |
| | CCCGTGCAGTGATACTACTGAGTACAACCCCTCT |
| | GTGAAGGGTCGGGTGACCATGCTGGTTGACACAA |
| | GCAAGAATCAATTTTCACTCCGGCTGTCATCTGTG |
| | ACACACTGCGCTCATTCAGGAAAACGGACGGTCG |
| | CCCTTATTACTACCA (SEQ ID NO: 467) |
| alemtuzumab-BtsI-20-4 | CCCTTTAATCAGATGCGTCGTGAGCCTTATGATTT |
| | CCCGTGCAGTGCTCCGGCTGTCATCTGTGACAGC |
| | TGCTGATACAGCAGTTTATTATTGCGCAAGGGAAG |
| | GACATACTGCCGCTCCTTTCGACTATTGGGGCCA |
| | GGCACTGCGCTCATTCAGGAAAACGGACGGTCGC |
| | CCTTATTACTACCA (SEQ ID NO: 468) |
| alemtuzumab-BtsI-20-5 | CCCTTTAATCAGATGCGTCGTGAGCCTTATGATTT |
| | CCCGTGCAGTGTCCTTTCGACTATTGGGGCCAGG |
| | GTTCACTCGTCACAGTCTCTTCAGGTGGGGCCGG |
| | CTCAGGAGCCGGGAGCGGGTCATCTGGAGCCGG |
| | CCACTGCGCTCATTCAGGAAAACGGACGGTCGCC |
| | CTTATTACTACCA (SEQ ID NO: 469) |
| alemtuzumab-BtsI-20-6 | CCCTTTAATCAGATGCGTCGTGAGCCTTATGATTT |
| | CCCGTGCAGTGGCGGGTCATCTGGAGCCGGCTCC |
| | GGGGATATCCAGATGACCCAGTCACCCTCTTCAC |
| | TCAGCGCCAGCGTGGGCGATCGCGTTACCATCAC |
| | ATGCCACTGCGCTCATTCAGGAAAACGGACGGTC |
| | GCCCTTATTACTACCA (SEQ ID NO: 470) |
| alemtuzumab-BtsI-20-7 | CCCTTTAATCAGATGCGTCGTGAGCCTTATGATTT |
| | CCCGTGCAGTGGGCGATCGCGTTACCATCACATG |
| | CAAAGCTTCTCAGAACATTGACAAATACCTGAATT |
| | GGTACCAACAGAAGCCCGGCAAGGCCCCCAAACT |
| | CCTCACTGCGCTCATTCAGGAAAACGGACGGTCG |
| | CCCTTATTACTACCA (SEQ ID NO: 471) |
| alemtuzumab-BtsI-20-8 | CCCTTTAATCAGATGCGTCGTGAGCCTTATGATTT |
| | CCCGTGCAGTGGGCAAGGCCCCCAAACTCCTCAT |
| | ATACAATACAAACAATCTGCAGACCGGCGTGCCA |
| | TCCCGCTTCTCAGGATCAGGCAGCGGCACTGACT |
| | TTACCACTGCGCTCATTCAGGAAAACGGACGGTC |
| | GCCCTTATTACTACCA (SEQ ID NO: 472) |
| alemtuzumab-BtsI-20-9 | CCCTTTAATCAGATGCGTCGTGAGCCTTATGATTT |
| | CCCGTGCAGTGGGCAGCGGCACTGACTTTACTTT |
| | CACAATCAGCAGCCTGCAACCAGAGGACATCGCC |
| | ACATATTACTGTCTCCAGCATATCTCCCGCCCTCG |
| | GACCACTGCGCTCATTCAGGAAAACGGACGGTCG |
| | CCCTTATTACTACCA (SEQ ID NO: 473) |
| alemtuzumab-BtsI-20-10 | CCCTTTAATCAGATGCGTCGTGAGCCTTATGATT |
| | TCCCGTGCAGTGGCATATCTCCCGCCCTCGGAC |
| | ATTCGGCCAAGGTACAAAGGTGGAGATTAAAGG |
| | GCCCTGAAGATATGACGACCCCTGTTCACTGCG |
| | CTCATTCAGGAAAACGGACGGTCGCCCTTATTA |
| | CTACCA (SEQ ID NO: 474) |
| bevacizumab-BtsI-20-0 | CCCTTTAATCAGATGCGTCGCGTTCTAAACGGCT |
| | AGATGCGCAGTGTTGTAAGATGGAAGCCGGGAT |
| | AGGCCCAGCCGGCCAGGCGCGAAGTGCAACTG |
| | GTTGAAAGCGGTGGGGGCCTGGTGCAGCCTGG |
| | TGGATCACTGCACTGCGGAAAGGGGAAAGACAG |
| | ACTGGTCGCCCTTATTACTACCA (SEQ ID NO: 475) |
| bevacizumab-BtsI-20-1 | CCCTTTAATCAGATGCGTCGCGTTCTAAACGGCT |
| | AGATGCGCAGTGGTGCAGCCTGGTGGATCACTG |
| | AGACTCTCCTGCGCCGCCAGCGGTTACACCTTC |
| | ACCAACTATGGTATGAATTGGGTTAGACAAGCAC |
| | CTGGAAACACTGCGGAAAGGGGAAAGACAGACT |
| | GGTCGCCCTTATTACTACCA (SEQ ID NO: 476) |
| bevacizumab-BtsI-20-2 | CCCTTTAATCAGATGCGTCGCGTTCTAAACGGCT |
| | AGATGCGCAGTGTGGGTTAGACAAGCACCTGGA |
| | AAGGGACTGGAGTGGGTTGGCTGGATAAATACA |
| | TATACAGGCGAGCCAACATATGCAGCTGACTTTA |
| | AGCGGACACTGCGGAAAGGGGAAAGACAGACT |
| | GGTCGCCCTTATTACTACCA (SEQ ID NO: 477) |
| bevacizumab-BtsI-20-3 | CCCTTTAATCAGATGCGTCGCGTTCTAAACGGCT |
| | AGATGCGCAGTGATATGCAGCTGACTTTAAGCG |
| | GAGGTTTACCTTCTCACTGGACACATCCAAGTCT |
| | ACTGCTTACCTGCAGATGAACTCACTCCGGGCTG |
| | AGGCACTGCGGAAAGGGGAAAGACAGACTGGTC |
| | GCCCTTATTACTACCA (SEQ ID NO: 478) |
| bevacizumab-BtsI-20-4 | CCCTTTAATCAGATGCGTCGCGTTCTAAACGGCT |
| | AGATGCGCAGTGTGAACTCACTCCGGGCTGAGG |
| | ATACAGCCGTTTACTATTGCGCCAAGTATCCCCA |
| | TTACTATGGTTCCAGCCACTGGTACTTCGATGTC |
| | TGGGGCCACTGCGGAAAGGGGAAAGACAGACT |
| | GGTCGCCCTTATTACTACCA (SEQ ID NO: 479) |
| bevacizumab-BtsI-20-5 | CCCTTTAATCAGATGCGTCGCGTTCTAAACGGCT |
| | AGATGCGCAGTGCACTGGTACTTCGATGTCTGG |
| | GGCCAGGGAACTCTGGTGACTGGGGGGTCCGG |
| | GGGCTCCGGAGGGGCCTCCGGAGCAGGATCCG |
| | GCGGACACTGCGGAAAGGGGAAAGACAGACTG |
| | GTCGCCCTTATTACTACCA (SEQ ID NO: 480) |
| bevacizumab-BtsI-20-6 | CCCTTTAATCAGATGCGTCGCGTTCTAAACGGC |
| | TAGATGCGCAGTGCGGAGCAGGATCCGGCGGA |
| | GGTGACATACAGATGACCCAGTCTCCATCCTCT |
| | CTGAGCGCCTCTGTGGGCGATCGCGTCACTAT |
| | TACCTGTTCTGCACTGCGGAAAGGGGAAAGAC |
| | AGACTGGTCGCCCTTATTACTACCA |
| | (SEQ ID NO: 481) |
| bevacizumab-BtsI-20-7 | CCCTTTAATCAGATGCGTCGCGTTCTAAACGGC |
| | TAGATGCGCAGTGATCGCGTCACTATTACCTGT |
| | TCTGCATCTCAGGATATTAGCAACTATCTGAAT |
| | TGGTATCAGCAGAAGCCAGGTAAGGCACCAAA |
| | AGTTCTGATCCACTGCGGAAAGGGGAAAGACA |
| | GACTGGTCGCCCTTATTACTACCA |
| | (SEQ ID NO: 482) |
| bevacizumab-BtsI-20-8 | CCCTTTAATCAGATGCGTCGCGTTCTAAACGGC |
| | TAGATGCGCAGTGAGGTAAGGCACCAAAAGTT |
| | CTGATCTACTTCACAAGCTCTCTGCATTCCGGG |
| | GTGCCCTCACGCTTCTCTGGTTCCGGCTCCGGG |
| | ACAGATTTCACACTGCGGAAAGGGGAAAGACA |
| | GACTGGTCGCCCTTATTACTACCA |
| | (SEQ ID NO: 483) |
| bevacizumab-BtsI-20-9 | CCCTTTAATCAGATGCGTCGCGTTCTAAACGGC |
| | TAGATGCGCAGTGCCGGCTCCGGGACAGATTT |
| | CACACTCACAATTTCCTCTCTGCAGCCCGAAGA |
| | TTTTGCAACTTACTACTGTCAGCAGTATTCTACA |
| | GTGCCATGGCACTGCGGAAAGGGGAAAGACAG |
| | ACTGGTCGCCCTTATTACTACCA |
| | (SEQ ID NO: 484) |
| bevacizumab-BtsI-20-10 | CCCTTTAATCAGATGCGTCGCGTTCTAAACGGC |
| | TAGATGCGCAGTGCAGCAGTATTCTACAGTGCC |
| | ATGGACTTTCGGACAGGGAACCAAGGTCGAGA |
| | TTAAAGGGCCCTTCCACAGCTCTATGAGGTGTT |
| | CACTGCGGAAAGGGGAAAGACAGACTGGTCGC |
| | CCTTATTACTACCA (SEQ ID NO: 485) |
| ranibizumab-BtsI-20-0 | CCCTTTAATCAGATGCGTCGGTATCCGAAGCGT |
| | GGAGTATGCAGTGTTGGTGTCGCAACATGATCT |
| | ACGGCCCAGCCGGCCAGGCGCGAAGTTCAGCT |
| | GGTTGAAAGCGGAGGTGGACTCGTGCAGCCCG |
| | GTGGGTCCCTGACACTGCTTGACTCCTACGCAT |
| | ACCTGGGTCGCCCTTATTACTACCA |
| | (SEQ ID NO: 486) |
| ranibizumab-BtsI-20-1 | CCCTTTAATCAGATGCGTCGGTATCCGAAGCGT |
| | GGAGTATGCAGTGAGCCCGGTGGGTCCCTGAG |
| | GCTCTCCTGCGCCGCTAGCGGATATGATTTCAC |
| | TCACTACGGTATGAATTGGGTCCGGCAGGCTCC |
| | CGGCAAAGGTCCACTGCTTGACTCCTACGCATA |
| | CCTGGGTCGCCCTTATTACTACCA |
| | (SEQ ID NO: 487) |
| ranibizumab-BtsI-20-2 | CCCTTTAATCAGATGCGTCGGTATCCGAAGCGT |
| | GGAGTATGCAGTGCAGGCTCCCGGCAAAGGTC |
| | TGGAATGGGTTGGCTGGATCAACACTTATACTG |
| | GGGAGCCTACCTACGCCGCCGATTTCAAGAGG |
| | CGCTTTACTTTCCACTGCTTGACTCCTACGCATA |
| | CCTGGGTCGCCCTTATTACTACCA |
| | (SEQ ID NO: 488) |
| ranibizumab-BtsI-20-3 | CCCTTTAATCAGATGCGTCGGTATCCGAAGCGT |
| | GGAGTATGCAGTGGATTTCAAGAGGCGCTTTAC |
| | TTTCTCACTCGATACCTCCAAATCCACAGCCTAT |
| | CTGCAAATGAATTCCCTGCGCGCCGAAGATACC |
| | GCAGTCTACCACTGCTTGACTCCTACGCATACC |
| | TGGGTCGCCCTTATTACTACCA |
| | (SEQ ID NO: 489) |
| ranibizumab-BtsI-20-4 | CCCTTTAATCAGATGCGTCGGTATCCGAAGCGT |
| | GGAGTATGCAGTGCGCCGAAGATACCGCAGTC |
| | TACTATTGTGCCAAGTATCCCTACTATTATGGGA |
| | CATCTCACTGGTACTTCGACGTGTGGGGGCAAG |
| | GGACTCTCGTCACTGCTTGACTCCTACGCATACC |
| | TGGGTCGCCCTTATTACTACCA |
| | (SEQ ID NO: 490) |
| ranibizumab-BtsI-20-5 | CCCTTTAATCAGATGCGTCGGTATCCGAAGCGT |
| | GGAGTATGCAGTGTGGGGGCAAGGGACTCTCG |
| | TCACTGTGTCTAGCGGGGGTAGCGCTGGGTCCG |
| | GCAGCAGCGGTGGGGCAAGCGGTAGCGGGGGC |
| | GACATTCAGCTGCACTGCTTGACTCCTACGCATA |
| | CCTGGGTCGCCCTTATTACTACCA |
| | (SEQ ID NO: 491) |
| ranibizumab-BtsI-20-6 | CCCTTTAATCAGATGCGTCGGTATCCGAAGCGT |
| | GGAGTATGCAGTGGCGGGGGCGACATTCAGCT |
| | GACACAAAGCCCCTCATCCCTGAGCGCTTCAGT |
| | GGGGGACCGCGTGACCATCACCTGTTCCGCCT |
| | CCCAGGACATCTCACTGCTTGACTCCTACGCAT |
| | ACCTGGGTCGCCCTTATTACTACCA |
| | (SEQ ID NO: 492) |
| ranibizumab-BtsI-20-7 | CCCTTTAATCAGATGCGTCGGTATCCGAAGCGT |
| | GGAGTATGCAGTGTTCCGCCTCCCAGGACATCT |
| | CAAACTACCTGAACTGGTACCAACAAAAACCTG |
| | GTAAAGCCCCTAAAGTTCTGATTTACTTCACAAG |
| | CTCTCTCCACCACTGCTTGACTCCTACGCATAC |
| | CTGGGTCGCCCTTATTACTACCA |
| | (SEQ ID NO: 493) |
| ranibizumab-BtsI-20-8 | CCCTTTAATCAGATGCGTCGGTATCCGAAGCGT |
| | GGAGTATGCAGTGGATTTACTTCACAAGCTCTC |
| | TCCACTCCGGCGTCCCTTCTAGGTTTTCTGGTA |
| | GCGGTAGCGGAACAGATTTCACTCTGACAATTA |
| | GCTCCCTCCACACTGCTTGACTCCTACGCATAC |
| | CTGGGTCGCCCTTATTACTACCA |
| | (SEQ ID NO: 494) |
| ranibizumab-BtsI-20-9 | CCCTTTAATCAGATGCGTCGGTATCCGAAGCGT |
| | GGAGTATGCAGTGCACTCTGACAATTAGCTCCC |
| | TCCAGCCTGAGGATTTTGCCACTTACTATTGTC |
| | AGCAGTATTCCACAGTGCCCTGGACTTTTGGGC |
| | AGGGCACCAACACTGCTTGACTCCTACGCATAC |
| | CTGGGTCGCCCTTATTACTACCA |
| | (SEQ ID NO: 495) |
| ranibizumab-BtsI-20-10 | CCCTTTAATCAGATGCGTCGGTATCCGAAGCGT |
| | GGAGTATGCAGTGACTTTTGGGCAGGGCACCA |
| | AGGTCGAAATCAAGGGGCCCGCAAACATGACT |
| | AGGAACCGTTCACTGCTTGACTCCTACGCATAC |
| | CTGGGTCGCCCTTATTACTACCA |
| | (SEQ ID NO: 496) |
| pertuzumab-BtsI-20-0 | CCCTTTAATCAGATGCGTCGCTTGTTATGGACG |
| | AGTTGCCGCAGTGTTGTGCTAAGTCACACTGTT |
| | GGGGCCCAGCCGGCCAGGCGCGAGGTCCAGC |
| | TGGTCGAGAGCGGCGGCGGGCTGGTTCAACCC |
| | GGGGGCTCACTGCCAGTATGAACGCGCCATTAA |
| | GGTCGCCCTTATTACTACCA (SEQ ID NO: 497) |
| pertuzumab-BtsI-20-1 | CCCTTTAATCAGATGCGTCGCTTGTTATGGACGA |
| | GTTGCCGCAGTGCTGGTTCAACCCGGGGGCTCC |
| | CTGCGGCTGTCATGTGCCGCCAGCGGCTTCACC |
| | TTTACTGATTACACAATGGACTGGGTGAGGCAGG |
| | CCCACTGCCAGTATGAACGCGCCATTAAGGTCGC |
| | CCTTATTACTACCA (SEQ ID NO: 498) |
| pertuzumab-BtsI-20-2 | CCCTTTAATCAGATGCGTCGCTTGTTATGGACGA |
| | GTTGCCGCAGTGTGGACTGGGTGAGGCAGGCCC |
| | CAGGAAAAGGCCTGGAATGGGTTGCCGACGTGA |
| | ATCCTAATTCCGGGGGTTCAATTTACAATCAGCG |
| | CTTTAAGGGCCACTGCCAGTATGAACGCGCCAT |
| | TAAGGTCGCCCTTATTACTACCA |
| | (SEQ ID NO: 499) |
| pertuzumab-BtsI-20-3 | CCCTTTAATCAGATGCGTCGCTTGTTATGGACGA |
| | GTTGCCGCAGTGTCAATTTACAATCAGCGCTTTA |
| | AGGGCCGGTTCACCCTGTCAGTCGACAGGAGCA |
| | AGAATACACTCTATCTCCAGATGAACTCCCTCCG |
| | CGCCACTGCCAGTATGAACGCGCCATTAAGGTCG |
| | CCCTTATTACTACCA (SEQ ID NO: 500) |
| pertuzumab-BtsI-20-4 | CCCTTTAATCAGATGCGTCGCTTGTTATGGACGA |
| | GTTGCCGCAGTGCCAGATGAACTCCCTCCGCGCT |
| | GAGGATACCGCCGTCTATTATTGTGCCCGCAATC |
| | TGGGTCCCTCTTTTTACTTTGACTATTGGGGCCAA |
| | GGGACACTGCCAGTATGAACGCGCCATTAAGGT |
| | CGCCCTTATTACTACCA (SEQ ID NO: 501) |
| pertuzumab-BtsI-20-5 | CCCTTTAATCAGATGCGTCGCTTGTTATGGACGA |
| | GTTGCCGCAGTGACTTTGACTATTGGGGCCAAG |
| | GGACCCTGGTCACCGTCTCTAGCGCCGGTGGCT |
| | CAGGAGGAAGCGGTGGCGCCTCTGGGGCTGGC |
| | AGCGGAGGACACTGCCAGTATGAACGCGCCATT |
| | AAGGTCGCCCTTATTACTACCA (SEQ ID NO: 502) |
| pertuzumab-BtsI-20-6 | CCCTTTAATCAGATGCGTCGCTTGTTATGGACGA |
| | GTTGCCGCAGTGGGGGCTGGCAGCGGAGGAGG |
| | CGACATTCAGATGACACAGAGCCCTAGCTCTCT |
| | CTCCGCTAGCGTGGGGGACAGGGTTACCATAAC |
| | TTGCAAGGCACACTGCCAGTATGAACGCGCCAT |
| | TAAGGTCGCCCTTATTACTACCA |
| | (SEQ ID NO: 503) |
| pertuzumab-BtsI-20-7 | CCCTTTAATCAGATGCGTCGCTTGTTATGGACGA |
| | GTTGCCGCAGTGCAGGGTTACCATAACTTGCAA |
| | GGCAAGCCAAGATGTCTCTATTGGTGTTGCTTG |
| | GTACCAGCAAAAGCCTGGAAAGGCTCCTAAACT |
| | GCTGATATCACTGCCAGTATGAACGCGCCATTA |
| | AGGTCGCCCTTATTACTACCA (SEQ ID NO: 504) |
| pertuzumab-BtsI-20-8 | CCCTTTAATCAGATGCGTCGCTTGTTATGGACGA |
| | GTTGCCGCAGTGGAAAGGCTCCTAAACTGCTGA |
| | TATACTCCGCCAGCTACAGGTATACAGGCGTGC |
| | CATCCCGGTTCTCAGGTTCCGGCTCAGGAACAG |
| | ATTTTACTCACTGCCAGTATGAACGCGCCATTAA |
| | GGTCGCCCTTATTACTACCA (SEQ ID NO: 505) |
| pertuzumab-BtsI-20-9 | CCCTTTAATCAGATGCGTCGCTTGTTATGGACGA |
| | GTTGCCGCAGTGTCCGGCTCAGGAACAGATTTT |
| | ACTCTCACCATTTCCAGCCTGCAACCCGAGGACT |
| | TCGCCACATACTATTGCCAGCAGTATTATATATAT |
| | CCTTACACTGCCAGTATGAACGCGCCATTAAGG |
| | TCGCCCTTATTACTACCA (SEQ ID NO: 506) |
| pertuzumab-BtsI-20-10 | CCCTTTAATCAGATGCGTCGCTTGTTATGGACGA |
| | GTTGCCGCAGTGTATTGCCAGCAGTATTATATAT |
| | ATCCTTACACTTTTGGTCAGGGTACTAAAGTGGA |
| | GATTAAAGGGCCCCCGGGACGAGATTAGTACAA |
| | TTCACTGCCAGTATGAACGCGCCATTAAGGTCGC |
| | CCTTATTACTACCA (SEQ ID NO: 507) |
| naptumomab-BtsI-20-0 | CCCTTTAATCAGATGCGTCGCCAAAGATTCAACC |
| | GTCCTGGCAGTGTTTCTAAACAGTTAGGCCCAGG |
| | GGCCCAGCCGGCCAGGCGCGAGGTGCAGCTCCA |
| | ACAATCTGGGCCTGATCTGGTTAAGCCAGGCGCT |
| | TCTGTGCACTGCTCCGTCCTGAAATGGCTAATGG |
| | TCGCCCTTATTACTACCA (SEQ ID NO: 508) |
| naptumomab-BtsI-20-1 | CCCTTTAATCAGATGCGTCGCCAAAGATTCAACC |
| | GTCCTGGCAGTGGGTTAAGCCAGGCGCTTCTGT |
| | GAAAATTTCCTGTAAGGCTTCAGGCTACAGCTT |
| | CACTGGCTATTATATGCATTGGGTGAAACAGTC |
| | TCCAGGACACTGCTCCGTCCTGAAATGGCTAAT |
| | GGTCGCCCTTATTACTACCA (SEQ ID NO: 509) |
| naptumomab-BtsI-20-2 | CCCTTTAATCAGATGCGTCGCCAAAGATTCAACC |
| | GTCCTGGCAGTGATTGGGTGAAACAGTCTCCAG |
| | GAAAGGGCCTGGAGTGGATTGGGCGGATCAATC |
| | CCAACAATGGAGTCACCCTCTACAATCAAAAATT |
| | CAAAGATCACTGCTCCGTCCTGAAATGGCTAATG |
| | GTCGCCCTTATTACTACCA (SEQ ID NO: 510) |
| naptumomab-BtsI-20-3 | CCCTTTAATCAGATGCGTCGCCAAAGATTCAACC |
| | GTCCTGGCAGTGTCACCCTCTACAATCAAAAATT |
| | CAAAGATAAAGCTACACTGACCGTCGATAAAAGC |
| | TCAACAACAGCCTACATGGAGCTGAGATCCCTCA |
| | CCTCCCACTGCTCCGTCCTGAAATGGCTAATGGT |
| | CGCCCTTATTACTACCA (SEQ ID NO: 511) |
| naptumomab-BtsI-20-4 | CCCTTTAATCAGATGCGTCGCCAAAGATTCAACC |
| | GTCCTGGCAGTGTGGAGCTGAGATCCCTCACCT |
| | CCGAGGACAGCGCTGTCTACTACTGCGCCAGGT |
| | CCACAATGATTACCAATTATGTGATGGACTACTG |
| | GGGTCAGCACTGCTCCGTCCTGAAATGGCTAAT |
| | GGTCGCCCTTATTACTACCA (SEQ ID NO: 512) |
| naptumomab-BtsI-20-5 | CCCTTTAATCAGATGCGTCGCCAAAGATTCAACC |
| | GTCCTGGCAGTGATGTGATGGACTACTGGGGTC |
| | AGGGAACCTCAGTGACCGTTAGCTCTGGCGGGT |
| | CCGCAGGTAGCGGCTCATCCGGCGGCGCATCCG |
| | GGAGCGGAGCACTGCTCCGTCCTGAAATGGCTA |
| | ATGGTCGCCCTTATTACTACCA (SEQ ID NO: 513) |
| naptumomab-BtsI-20-6 | CCCTTTAATCAGATGCGTCGCCAAAGATTCAACC |
| | GTCCTGGCAGTGGCGCATCCGGGAGCGGAGGG |
| | TCTATTGTCATGACACAGACCCCCACTTCCCTCC |
| | TGGTCTCTGCTGGCGACAGAGTCACAATCACTT |
| | GCAAGGCTCACTGCTCCGTCCTGAAATGGCTAA |
| | TGGTCGCCCTTATTACTACCA (SEQ ID NO: 514) |
| naptumomab-BtsI-20-7 | CCCTTTAATCAGATGCGTCGCCAAAGATTCAACC |
| | GTCCTGGCAGTGAGAGTCACAATCACTTGCAAGG |
| | CTAGCCAGAGCGTTTCAAACGACGTGGCATGGT |
| | ATCAACAGAAACCCGGCCAATCCCCCAAACTGCT |
| | GATTTCACTGCTCCGTCCTGAAATGGCTAATGG |
| | TCGCCCTTATTACTACCA (SEQ ID NO: 515) |
| naptumomab-BtsI-20-8 | CCCTTTAATCAGATGCGTCGCCAAAGATTCAACCG |
| | TCCTGGCAGTGCCAATCCCCCAAACTGCTGATTT |
| | CTTACACATCATCCAGATACGCCGGTGTGCCCGA |
| | TAGGTTTTCTGGTTCAGGGTATGGAACTGACTTC |
| | ACTCCACTGCTCCGTCCTGAAATGGCTAATGGTC |
| | GCCCTTATTACTACCA (SEQ ID NO: 516) |
| naptumomab-BtsI-20-9 | CCCTTTAATCAGATGCGTCGCCAAAGATTCAACC |
| | GTCCTGGCAGTGCAGGGTATGGAACTGACTTCAC |
| | TCTCACTATCTCTAGCGTTCAGGCTGAAGACGCT |
| | GCCGTCTACTTCTGCCAGCAAGACTACAACTCTC |
| | CTCCTCACTGCTCCGTCCTGAAATGGCTAATGGT |
| | CGCCCTTATTACTACCA (SEQ ID NO: 517) |
| naptumomab-BtsI-20-10 | CCCTTTAATCAGATGCGTCGCCAAAGATTCAACC |
| | GTCCTGGCAGTGCAGCAAGACTACAACTCTCCTC |
| | CTACATTCGGCGGGGGCACAAAGCTGGAGATCA |
| | AAGGGCCCCACGCCAGTTGTGAACATAATTCACT |
| | GCTCCGTCCTGAAATGGCTAATGGTCGCCCTTAT |
| | TACTACCA (SEQ ID NO: 518) |
| tadocizumab-BtsI-20-0 | CCCTTTAATCAGATGCGTCGTATTCATGCTTGGA |
| | CGGACTGCAGTGTTGTCTTTATACTTGCCTGCCG |
| | GGCCCAGCCGGCCAGGCGCCAGGTGCAGCTGG |
| | TGCAGTCCGGAGCCGAGGTCAAGAAGCCCGGA |
| | TCTTCCGTCACTGCTCCAACAAGCGGTACATAGT |
| | GGTCGCCCTTATTACTACCA (SEQ ID NO: 509) |
| tadocizumab-BtsI-20-1 | CCCTTTAATCAGATGCGTCGTATTCATGCTTGGA |
| | CGGACTGCAGTGGTCAAGAAGCCCGGATCTTCC |
| | GTCAAAGTCAGCTGCAAAGCTTCCGGTTATGCA |
| | TTCACTAACTACCTCATCGAGTGGGTCCGCCAG |
| | GCTCACTGCTCCAACAAGCGGTACATAGTGGTC |
| | GCCCTTATTACTACCA (SEQ ID NO: 520) |
| tadocizumab-BtsI-20-2 | CCCTTTAATCAGATGCGTCGTATTCATGCTTGG |
| | ACGGACTGCAGTGCGAGTGGGTCCGCCAGGCT |
| | CCAGGACAGGGACTGGAGTGGATTGGAGTGAT |
| | CTACCCTGGATCAGGAGGCACAAATTATAACG |
| | AGAAGTTTAAGGGCAGCACTGCTCCAACAAGC |
| | GGTACATAGTGGTCGCCCTTATTACTACCA |
| | (SEQ ID NO: 521) |
| tadocizumab-BtsI-20-3 | CCCTTTAATCAGATGCGTCGTATTCATGCTTGGA |
| | CGGACTGCAGTGCAAATTATAACGAGAAGTTTA |
| | AGGGCAGAGTCACTCTGACCGTCGATGAATCCA |
| | CAAATACAGCTTACATGGAGCTGTCATCACTCC |
| | GGAGCGCACTGCTCCAACAAGCGGTACATAGT |
| | GGTCGCCCTTATTACTACCA (SEQ ID NO: 522) |
| tadocizumab-BtsI-20-4 | CCCTTTAATCAGATGCGTCGTATTCATGCTTGGA |
| | CGGACTGCAGTGGAGCTGTCATCACTCCGGAGC |
| | GAGGACACAGCAGTTTATTTTTGCGCACGCCGC |
| | GATGGCAATTACGGGTGGTTCGCCTATTGGGGG |
| | CAGGGTACCACTGCTCCAACAAGCGGTACATAG |
| | TGGTCGCCCTTATTACTACCA (SEQ ID NO: 523) |
| tadocizumab-BtsI-20-5 | CCCTTTAATCAGATGCGTCGTATTCATGCTTGGA |
| | CGGACTGCAGTGCGCCTATTGGGGGCAGGGTAC |
| | TCTCGTCACCGTGTCATCAGGTGGGGCTGGCTC |
| | CGGGGCAGGTTCTGGCTCCTCCGGAGCTGGTTC |
| | AGGAGACACACTGCTCCAACAAGCGGTACATA |
| | GTGGTCGCCCTTATTACTACCA (SEQ ID NO: 524) |
| tadocizumab-BtsI-20-6 | CCCTTTAATCAGATGCGTCGTATTCATGCTTGGA |
| | CGGACTGCAGTGCCGGAGCTGGTTCAGGAGACA |
| | TCCAGATGACCCAGACACCCTCCACTCTCTCTGC |
| | TTCTGTGGGAGACAGAGTCACAATCAGCTGCCGG |
| | GCCACTGCTCCAACAAGCGGTACATAGTGGTCG |
| | CCCTTATTACTACCA (SEQ ID NO: 525) |
| tadocizumab-BtsI-20-7 | CCCTTTAATCAGATGCGTCGTATTCATGCTTGGA |
| | CGGACTGCAGTGGTCACAATCAGCTGCCGGGCT |
| | TCCCAGGATATAAACAACTACCTGAACTGGTACC |
| | AGCAGAAGCCTGGGAAGGCCCCCAAGCTGCTGA |
| | TCTACTACACTGCTCCAACAAGCGGTACATAGTG |
| | GTCGCCCTTATTACTACCA (SEQ ID NO: 526) |
| tadocizumab-BtsI-20-8 | CCCTTTAATCAGATGCGTCGTATTCATGCTTGGA |
| | CGGACTGCAGTGGCCCCCAAGCTGCTGATCTAC |
| | TATACATCCACTCTGCACAGCGGAGTTCCTAGCC |
| | GCTTCAGCGGATCCGGTAGCGGGACCGACTATA |
| | CCCTGACCACTGCTCCAACAAGCGGTACATAGT |
| | GGTCGCCCTTATTACTACCA (SEQ ID NO: 527) |
| tadocizumab-BtsI-20-9 | CCCTTTAATCAGATGCGTCGTATTCATGCTTGGA |
| | CGGACTGCAGTGGCGGGACCGACTATACCCTGA |
| | CCATCTCAAGCCTGCAGCCCGATGACTTCGCCAC |
| | ATACTTCTGTCAGCAGGGAAACACCCTCCCATGG |
| | ACATCACTGCTCCAACAAGCGGTACATAGTGGTC |
| | GCCCTTATTACTACCA (SEQ ID NO: 528) |
| tadocizumab-BtsI-20-10 | CCCTTTAATCAGATGCGTCGTATTCATGCTTGGA |
| | CGGACTGCAGTGGGAAACACCCTCCCATGGACA |
| | TTCGGTCAAGGAACTAAAGTTGAGGTTAAAGGG |
| | CCCCAAAGGCCAAATCAGTTCCATTCACTGCTCC |
| | AACAAGCGGTACATAGTGGTCGCCCTTATTACT |
| | ACCA (SEQ ID NO: 529) |
| efungumab-BtsI-20-0 | CCCTTTAATCAGATGCGTCGATCGACAATGGTAT |
| | GGCTGAGCAGTGTTCACCGCGATCAATACAACTT |
| | GGCCCAGCCGGCCAGGCGCGAAGTTCAACTGGT |
| | TGAGAGCGGTGCCGAGGTGAAGAAGCCTGGAGA |
| | GTCTCTCACTGCAGGAGTGGCTAGGAGACATAGG |
| | TCGCCCTTATTACTACCA (SEQ ID NO: 530) |
| efungumab-BtsI-20-1 | CCCTTTAATCAGATGCGTCGATCGACAATGGTAT |
| | GGCTGAGCAGTGGTGAAGAAGCCTGGAGAGTCT |
| | CTGAGAATTAGCTGTAAGGGCTCTGGCTGCATCA |
| | TCTCATCTTATTGGATTTCATGGGTTAGACAGAT |
| | GCCCGGCACTGCAGGAGTGGCTAGGAGACATA |
| | GGTCGCCCTTATTACTACCA (SEQ ID NO: 531) |
| efungumab-BtsI-20-2 | CCCTTTAATCAGATGCGTCGATCGACAATGGTAT |
| | GGCTGAGCAGTGTTCATGGGTTAGACAGATGCC |
| | CGGCAAAGGACTGGAATGGATGGGCAAGATAG |
| | ACCCTGGTGACTCCTACATCAATTATTCCCCTTCT |
| | TTTCAGGGGCCACTGCAGGAGTGGCTAGGAGAC |
| | ATAGGTCGCCCTTATTACTACCA (SEQ ID NO: 532) |
| efungumab-BtsI-20-3 | CCCTTTAATCAGATGCGTCGATCGACAATGGTAT |
| | GGCTGAGCAGTGTCAATTATTCCCCTTCTTTTCA |
| | GGGGCATGTCACAATCTCCGCAGACAAGAGCAT |
| | CAACACAGCATATCTCCAGTGGAATTCACTGAAA |
| | GCCTCCCACTGCAGGAGTGGCTAGGAGACATAG |
| | GTCGCCCTTATTACTACCA (SEQ ID NO: 533) |
| efungumab-BtsI-20-4 | CCCTTTAATCAGATGCGTCGATCGACAATGGTAT |
| | GGCTGAGCAGTGCAGTGGAATTCACTGAAAGCC |
| | TCCGACACAGCCATGTACTATTGCGCAAGAGGA |
| | GGGAGGGACTTCGGAGACTCTTTTGACTACTGG |
| | GGGCAGGCACTGCAGGAGTGGCTAGGAGACAT |
| | AGGTCGCCCTTATTACTACCA (SEQ ID NO: 534) |
| efungumab-BtsI-20-5 | CCCTTTAATCAGATGCGTCGATCGACAATGGTAT |
| | GGCTGAGCAGTGCTCTTTTGACTACTGGGGGCA |
| | GGGGACTCTGGTGACAGTGTCTAGCGGCGGGTC |
| | AGGAGGATCCGGTGGAGCCTCTGGCGCTGGAA |
| | GCGGCACTGCAGGAGTGGCTAGGAGACATAGG |
| | TCGCCCTTATTACTACCA (SEQ ID NO: 535) |
| efungumab-BtsI-20-6 | CCCTTTAATCAGATGCGTCGATCGACAATGGTAT |
| | GGCTGAGCAGTGCCTCTGGCGCTGGAAGCGGCG |
| | GCGGAGATGTGGTCATGACTCAATCCCCTTCCT |
| | TTCTGTCAGCATTCGTGGGCGATAGGATCACTA |
| | TTACTTGTCACTGCAGGAGTGGCTAGGAGACAT |
| | AGGTCGCCCTTATTACTACCA (SEQ ID NO: 536) |
| efungumab-BtsI-20-7 | CCCTTTAATCAGATGCGTCGATCGACAATGGTAT |
| | GGCTGAGCAGTGTGGGCGATAGGATCACTATTA |
| | CTTGTCGCGCCTCTTCTGGCATCTCCAGATATCT |
| | GGCTTGGTACCAGCAAGCTCCCGGAAAGGCCCC |
| | TAAGCTGCACTGCAGGAGTGGCTAGGAGACAT |
| | AGGTCGCCCTTATTACTACCA (SEQ ID NO: 537) |
| efungumab-BtsI-20-8 | CCCTTTAATCAGATGCGTCGATCGACAATGGTAT |
| | GGCTGAGCAGTGCCCGGAAAGGCCCCTAAGCTG |
| | CTCATATATGCCGCCTCCACCCTCCAGACTGGAG |
| | TGCCCAGCCGGTTTAGCGGTAGCGGTTCCGGTA |
| | CCGACACTGCAGGAGTGGCTAGGAGACATAGGT |
| | CGCCCTTATTACTACCA (SEQ ID NO: 538) |
| efungumab-BtsI-20-9 | CCCTTTAATCAGATGCGTCGATCGACAATGGTAT |
| | GGCTGAGCAGTGCGGTAGCGGTTCCGGTACCGA |
| | GTTTACCCTCACCATTAACTCTCTGCAGCCAGAA |
| | GACTTCGCCACATATTACTGTCAACACCTCAACT |
| | CCTATCCACTGCAGGAGTGGCTAGGAGACATAG |
| | GTCGCCCTTATTACTACCA (SEQ ID NO: 539) |
| efungumab-BtsI-20-10 | CCCTTTAATCAGATGCGTCGATCGACAATGGTAT |
| | GGCTGAGCAGTGACTGTCAACACCTCAACTCCTA |
| | TCCTCTCACTTTCGGCGGCGGGACCAAAGTCGA |
| | TATTAAGGGGCCCGGTGCATGGGAGGAACTAT |
| | ATTCACTGCAGGAGTGGCTAGGAGACATAGGTC |
| | GCCCTTATTACTACCA (SEQ ID NO: 540) |
| Abagovomab-BtsI-20-0 | CCCTTTAATCAGATGCGTCGGTCCTAGTGAGGAAT |
| | ACCGGGCAGTGTTTTCGGATAGACTCAGGAAGCG |
| | GCCCAGCCGGCCAGGCGCCAAGTTAAACTGCAG |
| | GAGAGCGGAGCCGAACTCGCCAGACCCGGAGCT |
| | TCTGTGCACTGCTAGGATCTGCGATTCTTCGGGG |
| | TCGCCCTTATTACTACCA (SEQ ID NO: 541) |
| Abagovomab-BtsI-20-1 | CCCTTTAATCAGATGCGTCGGTCCTAGTGAGGAAT |
| | ACCGGGCAGTGCCAGACCCGGAGCTTCTGTGAAA |
| | CTGAGCTGCAAAGCTTCTGGCTATACTTTTACCAA |
| | TTATTGGATGCAATGGGTGAAGCAGAGGCCAGGA |
| | CAGCACTGCTAGGATCTGCGATTCTTCGGGGTCGC |
| | CCTTATTACTACCA (SEQ ID NO: 542) |
| Abagovomab-BtsI-20-2 | CCCTTTAATCAGATGCGTCGGTCCTAGTGAGGAAT |
| | ACCGGGCAGTGGTGAAGCAGAGGCCAGGACAGG |
| | GACTGGACTGGATCGGAGCTATCTATCCTGGAGA |
| | CGGCAATACTCGGTACACACACAAATTTAAGGGG |
| | AAAGCTACACTGCTAGGATCTGCGATTCTTCGGGG |
| | TCGCCCTTATTACTACCA (SEQ ID NO: 543) |
| Abagovomab-BtsI-20-3 | CCCTTTAATCAGATGCGTCGGTCCTAGTGAGGAAT |
| | ACCGGGCAGTGCACACACAAATTTAAGGGGAAAG |
| | CTACCCTGACCGCTGATAAGTCATCATCTACCGCC |
| | TACATGCAGCTGAGCTCCCTGGCTTCAGAGGACAG |
| | CGCACTGCTAGGATCTGCGATTCTTCGGGGTCGC |
| | CCTTATTACTACCA (SEQ ID NO: 544) |
| Abagovomab-BtsI-20-4 | CCCTTTAATCAGATGCGTCGGTCCTAGTGAGGAA |
| | TACCGGGCAGTGTCCCTGGCTTCAGAGGACAGC |
| | GGCGTTTACTATTGCGCACGCGGCGAGGGAAAC |
| | TATGCATGGTTTGCATACTGGGGGCAGGGGACC |
| | ACCGTGACTCACTGCTAGGATCTGCGATTCTTCG |
| | GGGTCGCCCTTATTACTACCA (SEQ ID NO: 555) |
| Abagovomab-BtsI-20-5 | CCCTTTAATCAGATGCGTCGGTCCTAGTGAGGAAT |
| | ACCGGGCAGTGGGCAGGGGACCACCGTGACTGTG |
| | TCCTCAGGGGGGAGCGCTGGTAGCGGTTCCAGCG |
| | GCGGGGCCAGCGGTTCCGGGGGGGACATCGAGC |
| | TCACTCACTGCTAGGATCTGCGATTCTTCGGGGTC |
| | GCCCTTATTACTACCA (SEQ ID NO: 556) |
| Abagovomab-BtsI-20-6 | CCCTTTAATCAGATGCGTCGGTCCTAGTGAGGAAT |
| | ACCGGGCAGTGGGGGGGGACATCGAGCTCACTC |
| | AGTCTCCTGCAAGCCTGTCAGCATCAGTTGGGGA |
| | GACAGTTACCATCACCTGCCAGGCATCCGAAAATA |
| | TATACACTGCTAGGATCTGCGATTCTTCGGGGTCG |
| | CCCTTATTACTACCA (SEQ ID NO: 557) |
| Abagovomab-BtsI-20-7 | CCCTTTAATCAGATGCGTCGGTCCTAGTGAGGAAT |
| | ACCGGGCAGTGCTGCCAGGCATCCGAAAATATAT |
| | ACAGCTACCTCGCATGGCATCAGCAAAAGCAGGG |
| | TAAAAGCCCTCAGCTCCTGGTTTATAATGCTAAAA |
| | CCCCACTGCTAGGATCTGCGATTCTTCGGGGTCGC |
| | CCTTATTACTACCA (SEQ ID NO: 558) |
| Abagovomab-BtsI-20-8 | CCCTTTAATCAGATGCGTCGGTCCTAGTGAGGAAT |
| | ACCGGGCAGTGCAGCTCCTGGTTTATAATGCTAAA |
| | ACCCTGGCTGGAGGCGTCTCTTCAAGATTTAGCGG |
| | GAGCGGCTCCGGGACCCACTTCTCACTGAAAATA |
| | AACACTGCTAGGATCTGCGATTCTTCGGGGTCGC |
| | CCTTATTACTACCA (SEQ ID NO: 559) |
| Abagovomab-BtsI-20-9 | CCCTTTAATCAGATGCGTCGGTCCTAGTGAGGAAT |
| | ACCGGGCAGTGGGGACCCACTTCTCACTGAAAAT |
| | AAAGTCCCTGCAACCAGAGGATTTTGGTATTTACT |
| | ATTGTCAGCACCACTACGGCATACTCCCAACCTTC |
| | GGCACTGCTAGGATCTGCGATTCTTCGGGGTCGC |
| | CCTTATTACTACCA (SEQ ID NO: 560) |
| Abagovomab-BtsI-20-10 | CCCTTTAATCAGATGCGTCGGTCCTAGTGAGGAAT |
| | ACCGGGCAGTGTACGGCATACTCCCAACCTTCGGA |
| | GGGGGAACTAAGCTGGAAATCAAGGGGCCCTGC |
| | ATGGGTCTGTCTATTGTTTCACTGCTAGGATCTGC |
| | GATTCTTCGGGGTCGCCCTTATTACTACCA |
| | (SEQ ID NO: 561) |
| Motavizumab-BtsI-20-0 | CCCTTTAATCAGATGCGTCGTTAGATAGGTGTGTA |
| | GGCGCGCAGTGTTCCATTGATAGATTCGCTCGCG |
| | GCCCAGCCGGCCAGGCGCCAGGTTACCCTGCGC |
| | GAGAGCGGGCCTGCTCTGGTGAAACCCACTCAGA |
| | CCCTGCACTGCGTCAGCTAGTACGCACCTTAGGT |
| | CGCCCTTATTACTACCA (SEQ ID NO: 562) |
| Motavizumab-BtsI-20-1 | CCCTTTAATCAGATGCGTCGTTAGATAGGTGTGTA |
| | GGCGCGCAGTGTGGTGAAACCCACTCAGACCCTG |
| | ACTCTGACCTGCACATTCTCTGGCTTTTCCCTCTC |
| | TACTGCCGGAATGTCAGTGGGATGGATCCGCCAC |
| | ACTGCGTCAGCTAGTACGCACCTTAGGTCGCCCT |
| | TATTACTACCA (SEQ ID NO: 563) |
| Motavizumab-BtsI-20-2 | CCCTTTAATCAGATGCGTCGTTAGATAGGTGTGTA |
| | GGCGCGCAGTGTCAGTGGGATGGATCCGCCAGC |
| | CTCCTGGCAAAGCTCTGGAGTGGCTCGCTGATATT |
| | TGGTGGGACGATAAAAAGCATTATAATCCATCTCT |
| | GAAGGACCACTGCGTCAGCTAGTACGCACCTTAG |
| | GTCGCCCTTATTACTACCA (SEQ ID NO: 564) |
| Motavizumab-BtsI-20-3 | CCCTTTAATCAGATGCGTCGTTAGATAGGTGTGTA |
| | GGCGCGCAGTGAAAGCATTATAATCCATCTCTGAA |
| | GGACCGCCTCACCATCAGCAAGGACACTAGCAAG |
| | AATCAGGTGGTTCTCAAGGTGACCAATATGGACCC |
| | AGCACTGCGTCAGCTAGTACGCACCTTAGGTCGCC |
| | CTTATTACTACCA (SEQ ID NO: 565) |
| Motavizumab-BtsI-20-4 | CCCTTTAATCAGATGCGTCGTTAGATAGGTGTGTAG |
| | GCGCGCAGTGTCAAGGTGACCAATATGGACCCAGC |
| | TGATACCGCTACCTACTACTGTGCCAGGGACATGAT |
| | CTTCAACTTCTATTTTGACGTGTGGGGTCAGGGCAC |
| | TGCGTCAGCTAGTACGCACCTTAGGTCGCCCTTATT |
| | ACTACCA (SEQ ID NO: 566) |
| Motavizumab-BtsI-20-5 | CCCTTTAATCAGATGCGTCGTTAGATAGGTGTGTAG |
| | GCGCGCAGTGTATTTTGACGTGTGGGGTCAGGGCA |
| | CCACCGTCACCGTTAGCTCTGGGGGAGCCGGTAGC |
| | GGGGCCGGGAGCGGGAGCAGCGGCGCAGGCTCTG |
| | GAGCACTGCGTCAGCTAGTACGCACCTTAGGTCGCC |
| | CTTATTACTACCA (SEQ ID NO: 567) |
| Motavizumab-BtsI-20-6 | CCCTTTAATCAGATGCGTCGTTAGATAGGTGTGTAG |
| | GCGCGCAGTGGCGGCGCAGGCTCTGGAGATATACA |
| | GATGACTCAGAGCCCCTCTACCCTGTCTGCTTCCGT |
| | GGGCGACCGGGTCACCATCACATGCTCCGCCCACT |
| | GCGTCAGCTAGTACGCACCTTAGGTCGCCCTTATT |
| | ACTACCA (SEQ ID NO: 568) |
| Motavizumab-BtsI-20-7 | CCCTTTAATCAGATGCGTCGTTAGATAGGTGTGTAG |
| | GCGCGCAGTGGTCACCATCACATGCTCCGCCTCTAG |
| | CCGCGTCGGTTATATGCATTGGTACCAGCAGAAGC |
| | CCGGCAAGGCACCCAAACTCCTCATTTATGACACCA |
| | CTGCGTCAGCTAGTACGCACCTTAGGTCGCCCTTA |
| | TTACTACCA (SEQ ID NO: 569) |
| Motavizumab-BtsI-20-8 | CCCTTTAATCAGATGCGTCGTTAGATAGGTGTGTAG |
| | GCGCGCAGTGGCACCCAAACTCCTCATTTATGACAC |
| | CTCCAAGCTGGCCTCTGGAGTTCCCTCTCGGTTTTC |
| | CGGAAGCGGTAGCGGCACCGAGTTCACACTGACCA |
| | CTGCGTCAGCTAGTACGCACCTTAGGTCGCCCTTAT |
| | TACTACCA (SEQ ID NO: 570) |
| Motavizumab-BtsI-20-9 | CCCTTTAATCAGATGCGTCGTTAGATAGGTGTGTAG |
| | GCGCGCAGTGCGGCACCGAGTTCACACTGACCATC |
| | TCCTCTCTCCAGCCAGATGATTTCGCCACATATTATT |
| | GCTTCCAGGGCAGCGGGTATCCTTTTACATTTGCAC |
| | TGCGTCAGCTAGTACGCACCTTAGGTCGCCCTTATT |
| | ACTACCA (SEQ ID NO: 571) |
| Motavizumab-BtsI-20-10 | CCCTTTAATCAGATGCGTCGTTAGATAGGTGTGTAG |
| | GCGCGCAGTGGCAGCGGGTATCCTTTTACATTTGG |
| | TGGGGGAACTAAAGTGGAGATCAAAGGGCCCCTCC |
| | TATGCTAGCTCGACTCTTCACTGCGTCAGCTAGTAC |
| | GCACCTTAGGTCGCCCTTATTACTACCA |
| | (SEQ ID NO: 572) |
| bavituximab-BtsI-20-0 | CCCTTTAATCAGATGCGTCGTTCCGTTTATGCTTTC |
| | CAGCGCAGTGTTTTTTCTACTTTCCGGCTTGCGGC |
| | CCAGCCGGCCAGGCGCGAGGTGCAACTCCAGCAG |
| | TCTGGTCCCGAGCTGGAGAAGCCCGGCGCCCACT |
| | GCCTCGCTCTAAACTCCAAGGAGGTCGCCCTTATT |
| | ACTACCA (SEQ ID NO: 573) |
| bavituximab-BtsI-20-1 | CCCTTTAATCAGATGCGTCGTTCCGTTTATGCTTTC |
| | CAGCGCAGTGCTGGAGAAGCCCGGCGCCAGCGTG |
| | AAGCTGTCATGTAAAGCCAGCGGGTACTCATTCACT |
| | GGCTATAATATGAACTGGGTGAAACAGTCACATGG |
| | CACTGCCTCGCTCTAAACTCCAAGGAGGTCGCCCT |
| | TATTACTACCA (SEQ ID NO: 574) |
| bavituximab-BtsI-20-2 | CCCTTTAATCAGATGCGTCGTTCCGTTTATGCTTTC |
| | CAGCGCAGTGGAACTGGGTGAAACAGTCACATGG |
| | TAAGAGCCTGGAATGGATCGGCCATATTGACCCCT |
| | ATTACGGTGACACTTCTTATAACCAAAAATTCAGGG |
| | GTAACACTGCCTCGCTCTAAACTCCAAGGAGGTCG |
| | CCCTTATTACTACCA (SEQ ID NO: 575) |
| bavituximab-BtsI-20-3 | CCCTTTAATCAGATGCGTCGTTCCGTTTATGCTTTC |
| | CAGCGCAGTGCTTCTTATAACCAAAAATTCAGGGG |
| | TAAGGCCACCCTGACCGTGGACAAATCTAGCAGCA |
| | CAGCCTATATGCAGCTCAAATCCCTGACATCAGAA |
| | CACTGCCTCGCTCTAAACTCCAAGGAGGTCGCCCT |
| | TATTACTACCA (SEQ ID NO: 576) |
| bavituximab-BtsI-20-4 | CCCTTTAATCAGATGCGTCGTTCCGTTTATGCTTTC |
| | CAGCGCAGTGCAGCTCAAATCCCTGACATCAGAAG |
| | ACAGCGCTGTTTATTATTGTGTGAAAGGCGGGTAC |
| | TACGGTCATTGGTATTTCGACGTGTGGGGCGCCAC |
| | TGCCTCGCTCTAAACTCCAAGGAGGTCGCCCTTAT |
| | TACTACCA (SEQ ID NO: 577) |
| bavituximab-BtsI-20-5 | CCCTTTAATCAGATGCGTCGTTCCGTTTATGCTTTC |
| | CAGCGCAGTGGTATTTCGACGTGTGGGGCGCCGG |
| | GACCACTGTGACTGTGTCCTCTGGCGGATCTGGCG |
| | GCTCTGGCGGGGCCTCCGGAGCCGGATCTGGGGG |
| | CGCACTGCCTCGCTCTAAACTCCAAGGAGGTCGCC |
| | CTTATTACTACCA (SEQ ID NO: 578) |
| bavituximab-BtsI-20-6 | CCCTTTAATCAGATGCGTCGTTCCGTTTATGCTTTC |
| | CAGCGCAGTGGGAGCCGGATCTGGGGGCGGCGA |
| | CATTCAGATGACACAATCACCATCTTCTCTGTCCGC |
| | TTCCCTGGGTGAGCGCGTCTCCCTCACATGCCGGG |
| | CCACTGCCTCGCTCTAAACTCCAAGGAGGTCGCCC |
| | TTATTACTACCA (SEQ ID NO: 579) |
| bavituximab-BtsI-20-7 | CCCTTTAATCAGATGCGTCGTTCCGTTTATGCTTTC |
| | CAGCGCAGTGGTCTCCCTCACATGCCGGGCTTCTC |
| | AGGACATAGGCAGCTCCCTCAACTGGCTGCAACAG |
| | GGTCCAGACGGTACTATCAAGCGGCTCATTTATGC |
| | CACTGCCTCGCTCTAAACTCCAAGGAGGTCGCCCT |
| | TATTACTACCA (SEQ ID NO: 580) |
| bavituximab-BtsI-20-8 | CCCTTTAATCAGATGCGTCGTTCCGTTTATGCTTTC |
| | CAGCGCAGTGGTACTATCAAGCGGCTCATTTATGC |
| | TACCTCTAGCCTGGATTCAGGCGTGCCCAAAAGGT |
| | TTTCTGGATCTCGGTCCGGCTCAGACTATTCCCTC |
| | ACTGCCTCGCTCTAAACTCCAAGGAGGTCGCCCTT |
| | ATTACTACCA (SEQ ID NO: 581) |
| bavituximab-BtsI-20-9 | CCCTTTAATCAGATGCGTCGTTCCGTTTATGCTTTC |
| | CAGCGCAGTGCGGTCCGGCTCAGACTATTCCCTC |
| | ACTATTTCTTCTCTCGAAAGCGAGGATTTCGTGGA |
| | CTATTACTGTCTGCAGTACGTGAGCTCACCTCCTCA |
| | CTGCCTCGCTCTAAACTCCAAGGAGGTCGCCCTTA |
| | TTACTACCA (SEQ ID NO: 582) |
| bavituximab-BtsI-20-10 | CCCTTTAATCAGATGCGTCGTTCCGTTTATGCTTTC |
| | CAGCGCAGTGGCAGTACGTGAGCTCACCTCCTACT |
| | TTCGGGGCAGGCACCAAACTCGAACTGAAGGGGC |
| | CCATGGTAAGAAGCTCCCACAATTCACTGCCTCGC |
| | TCTAAACTCCAAGGAGGTCGCCCTTATTACTACCA |
| | (SEQ ID NO: 583) |
| lexatumumab-BtsI-20-0 | CCCTTTAATCAGATGCGTCGGTATAGTTTGTGCGGT |
| | GGTCGCAGTGTTATGACTATTGGGGTCGTACCGGC |
| | CCAGCCGGCCAGGCGCGAAGTTCAGCTGGTCCAGT |
| | CAGGAGGAGGGGTCGAACGGCCCGGCGGATCTCT |
| | GCACTGCCGAAGGTGTAGGGGATTGATGGTCGCCC |
| | TTATTACTACCA (SEQ ID NO: 584) |
| lexatumumab-BtsI-20-1 | CCCTTTAATCAGATGCGTCGGTATAGTTTGTGCGGT |
| | GGTCGCAGTGCGGCCCGGCGGATCTCTGCGGCTG |
| | TCCTGCGCCGCCAGCGGCTTCACATTCGATGATTA |
| | CGGTATGAGCTGGGTTAGACAAGCTCCAGGGAAAG |
| | GACACTGCCGAAGGTGTAGGGGATTGATGGTCGCC |
| | CTTATTACTACCA (SEQ ID NO: 585) |
| lexatumumab-BtsI-20-2 | CCCTTTAATCAGATGCGTCGGTATAGTTTGTGCGGT |
| | GGTCGCAGTGGGTTAGACAAGCTCCAGGGAAAGGA |
| | CTGGAGTGGGTGTCCGGCATCAATTGGAACGGTGG |
| | CAGCACAGGCTATGCTGATAGCGTCAAGGGCAGAG |
| | CACTGCCGAAGGTGTAGGGGATTGATGGTCGCCCT |
| | TATTACTACCA (SEQ ID NO: 586) |
| lexatumumab-BtsI-20-3 | CCCTTTAATCAGATGCGTCGGTATAGTTTGTGCGGT |
| | GGTCGCAGTGGCTGATAGCGTCAAGGGCAGAGTT |
| | ACAATCAGCAGAGACAATGCCAAGAACTCTCTGTA |
| | TCTCCAGATGAACTCCCTGAGGGCTGAAGATACCG |
| | CACTGCCGAAGGTGTAGGGGATTGATGGTCGCCCT |
| | TATTACTACCA (SEQ ID NO: 587) |
| lexatumumab-BtsI-20-4 | CCCTTTAATCAGATGCGTCGGTATAGTTTGTGCGGT |
| | GGTCGCAGTGCTCCCTGAGGGCTGAAGATACCGCA |
| | GTCTATTATTGCGCCAAAATTCTGGGAGCCGGAAG |
| | AGGATGGTACTTTGATCTCTGGGGGAAAGGAACTA |
| | CACTGCCGAAGGTGTAGGGGATTGATGGTCGCCCT |
| | TATTACTACCA (SEQ ID NO: 588) |
| lexatumumab-BtsI-20-5 | CCCTTTAATCAGATGCGTCGGTATAGTTTGTGCGGT |
| | GGTCGCAGTGTGATCTCTGGGGGAAAGGAACTACA |
| | GTCACAGTGTCTGGGGGCAGCGCAGGCAGCGGCT |
| | CCAGCGGCGGGGCTTCCGGATCAGGAGGGTCCTCC |
| | GCACTGCCGAAGGTGTAGGGGATTGATGGTCGCC |
| | CTTATTACTACCA (SEQ ID NO: 589) |
| lexatumumab-BtsI-20-6 | CCCTTTAATCAGATGCGTCGGTATAGTTTGTGCGGT |
| | GGTCGCAGTGTCCGGATCAGGAGGGTCCTCCGAGC |
| | TCACTCAGGACCCAGCTGTGTCTGTCGCCCTCGGGC |
| | AGACTGTGCGGATCACTTGTCAGGGAGATTCCCTCA |
| | CTGCCGAAGGTGTAGGGGATTGATGGTCGCCCTTA |
| | TTACTACCA (SEQ ID NO: 590) |
| lexatumumab-BtsI-20-7 | CCCTTTAATCAGATGCGTCGGTATAGTTTGTGCGGT |
| | GGTCGCAGTGGATCACTTGTCAGGGAGATTCCCTC |
| | CGCTCCTATTATGCCTCCTGGTACCAGCAGAAACCT |
| | GGCCAGGCCCCCGTGCTGGTCATCTACGGCAAAAC |
| | ACTGCCGAAGGTGTAGGGGATTGATGGTCGCCCT |
| | TATTACTACCA (SEQ ID NO: 591) |
| lexatumumab-BtsI-20-8 | CCCTTTAATCAGATGCGTCGGTATAGTTTGTGCGGT |
| | GGTCGCAGTGGTGCTGGTCATCTACGGCAAAAATA |
| | ATCGCCCATCAGGCATTCCCGACCGGTTTAGCGGA |
| | TCTTCTTCCGGGAATACTGCCTCTCTGACAATTACC |
| | ACTGCCGAAGGTGTAGGGGATTGATGGTCGCCCTT |
| | ATTACTACCA (SEQ ID NO: 592) |
| lexatumumab-BtsI-20-9 | CCCTTTAATCAGATGCGTCGGTATAGTTTGTGCGGT |
| | GGTCGCAGTGGGGAATACTGCCTCTCTGACAATTA |
| | CTGGTGCCCAAGCTGAGGATGAGGCCGATTACTAC |
| | TGTAACAGCCGCGACAGCTCAGGAAACCACGTGGT |
| | CACTGCCGAAGGTGTAGGGGATTGATGGTCGCCC |
| | TTATTACTACCA (SEQ ID NO: 593) |
| lexatumumab-BtsI-20-10 | CCCTTTAATCAGATGCGTCGGTATAGTTTGTGCGGT |
| | GGTCGCAGTGACAGCTCAGGAAACCACGTGGTGTT |
| | CGGGGGCGGAACTAAGCTCACCGTGCTGGGGCCCC |
| | TATGGTCATTCCCGTACGATTCACTGCCGAAGGTGT |
| | AGGGGATTGATGGTCGCCCTTATTACTACCA |
| | (SEQ ID NO: 594) |
| ibalizumab-BtsI-20-0 | CCCTTTAATCAGATGCGTCGTCAGCCTTTCATTGAT |
| | TGCGGCAGTGTTTCGACAATAGTTGAGCCCTTGGCC |
| | CAGCCGGCCAGGCGCCAGGTGCAGCTGCAACAAT |
| | CCGGCCCCGAGGTTGTGAAACCAGGCGCCTCTGCA |
| | CTGCCGAGCTACGGTATCAAGGAAGGTCGCCCTTA |
| | TTACTACCA (SEQ ID NO: 595) |
| ibalizumab-BtsI-20-1 | CCCTTTAATCAGATGCGTCGTCAGCCTTTCATTGAT |
| | TGCGGCAGTGTGTGAAACCAGGCGCCTCTGTGAAG |
| | ATGTCTTGCAAGGCCTCAGGCTATACATTCACCAGC |
| | TATGTGATTCACTGGGTGCGCCAGAAACCAGGCAC |
| | TGCCGAGCTACGGTATCAAGGAAGGTCGCCCTTAT |
| | TACTACCA (SEQ ID NO: 596) |
| ibalizumab-BtsI-20-2 | CCCTTTAATCAGATGCGTCGTCAGCCTTTCATTGAT |
| | TGCGGCAGTGTGGGTGCGCCAGAAACCAGGACAG |
| | GGTCTCGATTGGATTGGCTATATTAACCCTTACAAT |
| | GATGGTACAGACTATGACGAGAAGTTTAAAGGCAA |
| | GGCACTGCCGAGCTACGGTATCAAGGAAGGTCGCC |
| | CTTATTACTACCA (SEQ ID NO: 597) |
| ibalizumab-BtsI-20-3 | CCCTTTAATCAGATGCGTCGTCAGCCTTTCATTGAT |
| | TGCGGCAGTGTATGACGAGAAGTTTAAAGGCAAGG |
| | CCACACTGACAAGCGATACCTCTACTAGCACCGCC |
| | TATATGGAGCTCAGCTCCCTCCGGTCAGAAGACAC |
| | CGCACTGCCGAGCTACGGTATCAAGGAAGGTCGCC |
| | CTTATTACTACCA (SEQ ID NO: 598) |
| ibalizumab-BtsI-20-4 | CCCTTTAATCAGATGCGTCGTCAGCCTTTCATTGAT |
| | TGCGGCAGTGTCCCTCCGGTCAGAAGACACCGCTG |
| | TGTATTATTGTGCCAGAGAAAAAGATAATTATGCTA |
| | CAGGCGCTTGGTTCGCCTACTGGGGACAGGGGAC |
| | TCCACTGCCGAGCTACGGTATCAAGGAAGGTCGCC |
| | CTTATTACTACCA (SEQ ID NO: 599) |
| ibalizumab-BtsI-20-5 | CCCTTTAATCAGATGCGTCGTCAGCCTTTCATTGAT |
| | TGCGGCAGTGGCCTACTGGGGACAGGGGACTCTC |
| | GTGACTGTGTCAAGCGGTGGAGCCGGGTCCGGCG |
| | CCGGCTCTGGTTCCAGCGGGGCCGGTTCCGGGGA |
| | CATTGTCACTGCCGAGCTACGGTATCAAGGAAGGT |
| | CGCCCTTATTACTACCA (SEQ ID NO: 600) |
| ibalizumab-BtsI-20-6 | CCCTTTAATCAGATGCGTCGTCAGCCTTTCATTGAT |
| | TGCGGCAGTGGCCGGTTCCGGGGACATTGTGATG |
| | ACCCAGTCTCCAGATAGCCTGGCTGTGTCTCTGGG |
| | CGAGAGGGTGACAATGAATTGTAAGTCCTCACAAA |
| | GCCTCCACTGCCGAGCTACGGTATCAAGGAAGGT |
| | CGCCCTTATTACTACCA (SEQ ID NO: 601) |
| ibalizumab-BtsI-20-7 | CCCTTTAATCAGATGCGTCGTCAGCCTTTCATTGAT |
| | TGCGGCAGTGTGAATTGTAAGTCCTCACAAAGCCT |
| | CCTGTATTCTACCAATCAGAAGAACTACCTGGCTTG |
| | GTATCAACAGAAGCCAGGCCAATCTCCCAAGCTCC |
| | TCACTGCCGAGCTACGGTATCAAGGAAGGTCGCCC |
| | TTATTACTACCA (SEQ ID NO: 602) |
| ibalizumab-BtsI-20-8 | CCCTTTAATCAGATGCGTCGTCAGCCTTTCATTGAT |
| | TGCGGCAGTGCAGGCCAATCTCCCAAGCTCCTCAT |
| | TTATTGGGCTTCCACAAGGGAGTCCGGCGTGCCAG |
| | ACCGGTTTAGCGGATCCGGCTCCGGCACTGATTTC |
| | ACCACTGCCGAGCTACGGTATCAAGGAAGGTCGCC |
| | CTTATTACTACCA (SEQ ID NO: 603) |
| ibalizumab-BtsI-20-9 | CCCTTTAATCAGATGCGTCGTCAGCCTTTCATTGAT |
| | TGCGGCAGTGCGGCTCCGGCACTGATTTCACCCTC |
| | ACCATCAGCTCCGTTCAAGCCGAAGATGTGGCCGT |
| | CTACTACTGCCAGCAATATTATTCCTATCGCACCTT |
| | TCACTGCCGAGCTACGGTATCAAGGAAGGTCGCCC |
| | TTATTACTACCA (SEQ ID NO: 604) |
| ibalizumab-BtsI-20-10 | CCCTTTAATCAGATGCGTCGTCAGCCTTTCATTGAT |
| | TGCGGCAGTGCAGCAATATTATTCCTATCGCACCTT |
| | TGGCGGAGGGACTAAACTGGAGATTAAGGGGCCC |
| | TAATCGGCTACGTTGTGTCTTTCACTGCCGAGCTAC |
| | GGTATCAAGGAAGGTCGCCCTTATTACTACCA |
| | (SEQ ID NO: 605) |
| tenatumomab-BtsI-20-0 | CCCTTTAATCAGATGCGTCGAGGGTCGTGGTTAAA |
| | GGTACGCAGTGTTGAGCCATGTGAAATGTGTGTGG |
| | CCCAGCCGGCCAGGCGCGAGATCCAACTCCAGCA |
| | GTCTGGACCTGAGCTGGTGAAGCCAGGTGCCTCTG |
| | CACTGCCTAACGACCGGAAAGAAACGGGTCGCCCT |
| | TATTACTACCA (SEQ ID NO: 606) |
| tenatumomab-BtsI-20-1 | CCCTTTAATCAGATGCGTCGAGGGTCGTGGTTAAAG |
| | GTACGCAGTGGGTGAAGCCAGGTGCCTCTGTGAAG |
| | GTGTCATGCAAAGCTTCCGGCTATGCATTTACATCT |
| | TACAATATGTATTGGGTGAAGCAATCACATGGCAAG |
| | CACTGCCTAACGACCGGAAAGAAACGGGTCGCCCT |
| | TATTACTACCA (SEQ ID NO: 607) |
| tenatumomab-BtsI-20-2 | CCCTTTAATCAGATGCGTCGAGGGTCGTGGTTAAAG |
| | GTACGCAGTGGGGTGAAGCAATCACATGGCAAGAG |
| | CCTGGAGTGGATTGGCTATATTGATCCATATAATGG |
| | CGTGACCTCTTACAACCAGAAATTCAAGGGGAAGG |
| | CCACTGCCTAACGACCGGAAAGAAACGGGTCGCCC |
| | TTATTACTACCA (SEQ ID NO: 608) |
| tenatumomab-BtsI-20-3 | CCCTTTAATCAGATGCGTCGAGGGTCGTGGTTAAAG |
| | GTACGCAGTGCAACCAGAAATTCAAGGGGAAGGCT |
| | ACCCTCACAGTTGACAAGTCTTCTTCTACTGCCTATA |
| | TGCACCTCAATTCACTGACATCTGAGGACTCTGCCC |
| | ACTGCCTAACGACCGGAAAGAAACGGGTCGCCCTTA |
| | TTACTACCA (SEQ ID NO: 609) |
| tenatumomab-BtsI-20-4 | CCCTTTAATCAGATGCGTCGAGGGTCGTGGTTAAAG |
| | GTACGCAGTGTCACTGACATCTGAGGACTCTGCCGT |
| | GTATTATTGCGCTAGGGGTGGAGGAAGCATCTACTA |
| | TGCCATGGACTATTGGGGACAAGGGACCAGCGCAC |
| | TGCCTAACGACCGGAAAGAAACGGGTCGCCCTTATT |
| | ACTACCA (SEQ ID NO: 610) |
| tenatumomab-BtsI-20-5 | CCCTTTAATCAGATGCGTCGAGGGTCGTGGTTAAAG |
| | GTACGCAGTGATTGGGGACAAGGGACCAGCGTGAC |
| | TGTCTCAAGCGGCGGCTCTGGCGGCAGCGGCGGCG |
| | CCAGCGGCGCAGGCTCCGGGGGGGGAGATATTGT |
| | GATCACTGCCTAACGACCGGAAAGAAACGGGTCGC |
| | CCTTATTACTACCA (SEQ ID NO: 611) |
| tenatumomab-BtsI-20-6 | CCCTTTAATCAGATGCGTCGAGGGTCGTGGTTAAAG |
| | GTACGCAGTGCCGGGGGGGGAGATATTGTGATGAC |
| | ACAGGCCGCACCTTCCGTGCCTGTGACCCCTGGGG |
| | AGTCAGTGAGCATCAGCTGCCGCTCCTCCAAGTCC |
| | CTCACTGCCTAACGACCGGAAAGAAACGGGTCGCC |
| | CTTATTACTACCA (SEQ ID NO: 612) |
| tenatumomab-BtsI-20-7 | CCCTTTAATCAGATGCGTCGAGGGTCGTGGTTAAAG |
| | GTACGCAGTGTGCCGCTCCTCCAAGTCCCTGCTGCA |
| | TTCCAATGGCAATACCTATCTCTATTGGTTCCTCCAG |
| | AGACCAGGACAATCCCCACAGCTGCTGATCTACACA |
| | CTGCCTAACGACCGGAAAGAAACGGGTCGCCCTTAT |
| | TACTACCA (SEQ ID NO: 613) |
| tenatumomab-BtsI-20-8 | CCCTTTAATCAGATGCGTCGAGGGTCGTGGTTAAAG |
| | GTACGCAGTGTCCCCACAGCTGCTGATCTACAGAAT |
| | GTCCAACCTCGCATCTGGAGTCCCTGACCGGTTCTC |
| | AGGCAGCGGTAGCGGCACCGCATTTACTCTGCGCAC |
| | TGCCTAACGACCGGAAAGAAACGGGTCGCCCTTATT |
| | ACTACCA (SEQ ID NO: 614) |
| tenatumomab-BtsI-20-9 | CCCTTTAATCAGATGCGTCGAGGGTCGTGGTTAAAG |
| | GTACGCAGTGGCGGCACCGCATTTACTCTGCGGATT |
| | TCTAGGGTGGAGGCCGAAGATGTGGGTGTGTACTA |
| | CTGTATGCAACACCTGGAGTATCCCCTGACTTTTGG |
| | CACTGCCTAACGACCGGAAAGAAACGGGTCGCCCT |
| | TATTACTACCA (SEQ ID NO: 615) |
| tenatumomab-BtsI-20-10 | CCCTTTAATCAGATGCGTCGAGGGTCGTGGTTAAAG |
| | GTACGCAGTGCCTGGAGTATCCCCTGACTTTTGGAG |
| | CCGGAACCAAGCTCGAACTGAAGGGGCCCTGACTC |
| | GATCCTTTAGTCCGTTCACTGCCTAACGACCGGAAA |
| | GAAACGGGTCGCCCTTATTACTACCA |
| | (SEQ ID NO: 616) |
| canakinumab-BtsI-20-0 | CCCTTTAATCAGATGCGTCGTGCAAGTGTACAAAT |
| | CCAGCGCAGTGTTCGTATACGTAAGGGTTCCGAG |
| | GCCCAGCCGGCCAGGCGCCAGGTGCAACTCGTG |
| | GAATCTGGAGGCGGCGTCGTGCAGCCCGGGAGG |
| | TCTCTGCACTGCTAGGAAAGGGATCACCGTTCGG |
| | TCGCCCTTATTACTACCA (SEQ ID NO: 617) |
| canakinumab-BtsI-20-1 | CCCTTTAATCAGATGCGTCGTGCAAGTGTACAAAT |
| | CCAGCGCAGTGGCAGCCCGGGAGGTCTCTGCGGC |
| | TGTCATGTGCAGCTTCAGGCTTCACTTTCAGCGTC |
| | TATGGTATGAACTGGGTGAGACAGGCACCTGGAA |
| | AAGCACTGCTAGGAAAGGGATCACCGTTCGGTCG |
| | CCCTTATTACTACCA (SEQ ID NO: 618) |
| canakinumab-BtsI-20-2 | CCCTTTAATCAGATGCGTCGTGCAAGTGTACAAAT |
| | CCAGCGCAGTGGTGAGACAGGCACCTGGAAAAGG |
| | ACTCGAATGGGTGGCCATCATCTGGTACGACGGC |
| | GACAACCAATACTACGCCGACTCCGTCAAGGGGA |
| | GATTCACTGCTAGGAAAGGGATCACCGTTCGGTC |
| | GCCCTTATTACTACCA (SEQ ID NO: 619) |
| canakinumab-BtsI-20-3 | CCCTTTAATCAGATGCGTCGTGCAAGTGTACAAAT |
| | CCAGCGCAGTGCCGACTCCGTCAAGGGGAGATTC |
| | ACAATTTCACGCGATAACTCCAAAAATACACTGTA |
| | CCTCCAGATGAACGGCCTGAGAGCTGAGGACACA |
| | GCACTGCTAGGAAAGGGATCACCGTTCGGTCGCC |
| | CTTATTACTACCA (SEQ ID NO: 620) |
| canakinumab-BtsI-20-4 | CCCTTTAATCAGATGCGTCGTGCAAGTGTACAAAT |
| | CCAGCGCAGTGGGCCTGAGAGCTGAGGACACAG |
| | CCGTTTATTACTGTGCCAGGGACCTCCGGACCGG |
| | ACCCTTCGACTATTGGGGACAGGGGACACTGGTC |
| | ACAGTCACTGCTAGGAAAGGGATCACCGTTCGGT |
| | CGCCCTTATTACTACCA (SEQ ID NO: 621) |
| canakinumab-BtsI-20-5 | CCCTTTAATCAGATGCGTCGTGCAAGTGTACAAAT |
| | CCAGCGCAGTGACAGGGGACACTGGTCACAGTGT |
| | CAAGCGCTTCCGGAGGGTCTGCAGGGTCCGGATC |
| | CAGCGGGGGGGCTTCAGGGAGCGGAGGGGAGAT |
| | CGTTCCACTGCTAGGAAAGGGATCACCGTTCGGT |
| | CGCCCTTATTACTACCA (SEQ ID NO: 622) |
| canakinumab-BtsI-20-6 | CCCTTTAATCAGATGCGTCGTGCAAGTGTACAAAT |
| | CCAGCGCAGTGGAGCGGAGGGGAGATCGTTCTGA |
| | CTCAGTCTCCAGACTTTCAGTCTGTCACACCAAAG |
| | GAAAAGGTCACCATCACTTGCCGGGCCTCACAATC |
| | CACACTGCTAGGAAAGGGATCACCGTTCGGTCGC |
| | CCTTATTACTACCA (SEQ ID NO: 623) |
| canakinumab-BtsI-20-7 | CCCTTTAATCAGATGCGTCGTGCAAGTGTACAAAT |
| | CCAGCGCAGTGTTGCCGGGCCTCACAATCCATCG |
| | GTTCTAGCCTGCACTGGTATCAGCAGAAACCAGAC |
| | CAGTCCCCCAAGCTGCTCATCAAGTACGCTTCACA |
| | GTCACTGCTAGGAAAGGGATCACCGTTCGGTCGC |
| | CCTTATTACTACCA |
| canakinumab-BtsI-20-8 | CCCTTTAATCAGATGCGTCGTGCAAGTGTACAAAT |
| | CCAGCGCAGTGTGCTCATCAAGTACGCTTCACAGT |
| | CTTTCAGCGGCGTCCCATCCAGGTTCTCCGGCTCC |
| | GGTTCCGGCACAGACTTCACTCTGACCATCAATAG |
| | CCTCACTGCTAGGAAAGGGATCACCGTTCGGTCGC |
| | CCTTATTACTACCA (SEQ ID NO: 624) |
| canakinumab-BtsI-20-9 | CCCTTTAATCAGATGCGTCGTGCAAGTGTACAAAT |
| | CCAGCGCAGTGGACTTCACTCTGACCATCAATAGC |
| | CTCGAAGCTGAAGACGCTGCTGCTTATTACTGTC |
| | ACCAAAGCAGCTCTCTGCCCTTTACTTTTGGTCC |
| | TGGCACTGCTAGGAAAGGGATCACCGTTCGGTC |
| | GCCCTTATTACTACCA (SEQ ID NO: 625) |
| canakinumab-BtsI-20-10 | CCCTTTAATCAGATGCGTCGTGCAAGTGTACAAAT |
| | CCAGCGCAGTGTCTGCCCTTTACTTTTGGTCCTGG |
| | CACAAAGGTGGACATTAAGGGGCCCACGCTTTGT |
| | GTTATCCGATGTTCACTGCTAGGAAAGGGATCAC |
| | CGTTCGGTCGCCCTTATTACTACCA |
| | (SEQ ID NO: 626) |
| etaracizumab-BtsI-20-0 | CCCTTTAATCAGATGCGTCGCTTAAGGTTTGCCCA |
| | TTCCCGCAGTGTTTTATGATGTCCGGATACCCGG |
| | GCCCAGCCGGCCAGGCGCCAGGTGCAGCTGGTG |
| | GAAAGCGGTGGCGGTGTCGTGCAGCCCGGCCGC |
| | AGCCTGAGACTCACTGCACACCGTGGAAGCTATA |
| | ACAGGTCGCCCTTATTACTACCA |
| | (SEQ ID NO: 627) |
| etaracizumab-BtsI-20-1 | CCCTTTAATCAGATGCGTCGCTTAAGGTTTGCCCA |
| | TTCCCGCAGTGCGGCCGCAGCCTGAGACTCTCCT |
| | GCGCTGCATCAGGTTTTACATTTTCTAGCTACGAT |
| | ATGTCTTGGGTCCGGCAGGCACCAGGAAAGGGGC |
| | TGGAGTGGGCACTGCACACCGTGGAAGCTATAA |
| | CAGGTCGCCCTTATTACTACCA |
| | (SEQ ID NO: 628) |
| etaracizumab-BtsI-20-2 | CCCTTTAATCAGATGCGTCGCTTAAGGTTTGCCCA |
| | TTCCCGCAGTGCAGGAAAGGGGCTGGAGTGGGT |
| | GGCTAAAGTTTCTTCCGGAGGGGGGAGCACCTA |
| | CTATCTCGACACTGTTCAGGGCCGGTTCACTATA |
| | TCCCGGGACAATCACTGCACACCGTGGAAGCTA |
| | TAACAGGTCGCCCTTATTACTACCA |
| | (SEQ ID NO: 629) |
| etaracizumab-BtsI-20-3 | CCCTTTAATCAGATGCGTCGCTTAAGGTTTGCCCA |
| | TTCCCGCAGTGCGGTTCACTATATCCCGGGACAA |
| | TTCTAAGAATACACTGTACCTGCAGATGAATTCTC |
| | TGAGGGCAGAAGATACCGCTGTGTACTATTGTGC |
| | ACGGCATCTCACTGCACACCGTGGAAGCTATAAC |
| | AGGTCGCCCTTATTACTACCA (SEQ ID NO: 630) |
| etaracizumab-BtsI-20-4 | CCCTTTAATCAGATGCGTCGCTTAAGGTTTGCCCA |
| | TTCCCGCAGTGTGTGTACTATTGTGCACGGCATCT |
| | GCACGGATCCTTCGCTTCCTGGGGACAGGGCACT |
| | ACTGTCACCGTTTCTAGCGGCGGTGCTGGATCTG |
| | GAGCTGGATCACTGCACACCGTGGAAGCTATAAC |
| | AGGTCGCCCTTATTACTACCA (SEQ ID NO: 631) |
| etaracizumab-BtsI-20-5 | CCCTTTAATCAGATGCGTCGCTTAAGGTTTGCCCA |
| | TTCCCGCAGTGGTGCTGGATCTGGAGCTGGATCA |
| | GGGTCCTCTGGAGCTGGCTCAGGTGAGATCGTGC |
| | TGACCCAAAGCCCTGCTACCCTGAGCCTCTCCCCA |
| | GGAGAGCACTGCACACCGTGGAAGCTATAACAGG |
| | TCGCCCTTATTACTACCA (SEQ ID NO: 632) |
| etaracizumab-BtsI-20-6 | CCCTTTAATCAGATGCGTCGCTTAAGGTTTGCCCA |
| | TTCCCGCAGTGCTGAGCCTCTCCCCAGGAGAGCG |
| | GGCAACACTGTCTTGTCAGGCATCTCAATCAATTA |
| | GCAACTTCCTGCATTGGTACCAACAGCGGCCAGG |
| | CCACACTGCACACCGTGGAAGCTATAACAGGTCG |
| | CCCTTATTACTACCA (SEQ ID NO: 633) |
| etaracizumab-BtsI-20-7 | CCCTTTAATCAGATGCGTCGCTTAAGGTTTGCCCA |
| | TTCCCGCAGTGCCAACAGCGGCCAGGCCAAGCCC |
| | CTAGGCTGCTCATTAGATACAGGTCCCAATCAATT |
| | AGCGGAATACCAGCCAGGTTTTCCGGCTCTGGAT |
| | CCGGTACCGCACTGCACACCGTGGAAGCTATAAC |
| | AGGTCGCCCTTATTACTACCA (SEQ ID NO: 634) |
| etaracizumab-BtsI-20-8 | CCCTTTAATCAGATGCGTCGCTTAAGGTTTGCCCA |
| | TTCCCGCAGTGCCGGCTCTGGATCCGGTACCGAC |
| | TTCACCCTCACCATCTCTTCCCTGGAACCCGAAGA |
| | CTTCGCCGTGTATTACTGTCAGCAGTCTGGGTCTT |
| | GGCCTCTGCACTGCACACCGTGGAAGCTATAACA |
| | GGTCGCCCTTATTACTACCA (SEQ ID NO: 635) |
| etaracizumab-BtsI-20-9 | CCCTTTAATCAGATGCGTCGCTTAAGGTTTGCCCA |
| | TTCCCGCAGTGCAGTCTGGGTCTTGGCCTCTGACA |
| | TTCGGAGGTGGAACTAAAGTGGAAATCAAAGGGC |
| | CCACCACGGTGGAGTATACATCTTCACTGCACAC |
| | CGTGGAAGCTATAACAGGTCGCCCTTATTACTACCA |
| | (SEQ ID NO: 636) |
| otelixizumab-BtsI-20-0 | CCCTTTAATCAGATGCGTCGTGGTTCGTTAGTCGA |
| | TCTCCGCAGTGTTTCTTAGAAATCCACGGGTCCGG |
| | CCCAGCCGGCCAGGCGCGAAGTGCAGCTGCTGG |
| | AAAGCGGCGGCGGGCTGGTCCAGCCCGGCGGAT |
| | CCCTGACACTGCGACCCAGTAAAATCCCGTCTGG |
| | TCGCCCTTATTACTACCA (SEQ ID NO: 637) |
| otelixizumab-BtsI-20-1 | CCCTTTAATCAGATGCGTCGTGGTTCGTTAGTCGA |
| | TCTCCGCAGTGAGCCCGGCGGATCCCTGAGACTG |
| | TCATGTGCCGCCAGCGGTTTCACTTTTAGCTCATT |
| | TCCAATGGCCTGGGTTCGGCAGGCACCAGGAAAA |
| | GGCCCACTGCGACCCAGTAAAATCCCGTCTGGTC |
| | GCCCTTATTACTACCA (SEQ ID NO: 638) |
| otelixizumab-BtsI-20-2 | CCCTTTAATCAGATGCGTCGTGGTTCGTTAGTCGA |
| | TCTCCGCAGTGGGCAGGCACCAGGAAAAGGCCT |
| | CGAATGGGTGTCCACAATATCAACTTCTGGCGGT |
| | AGAACATACTATAGGGACTCCGTGAAGGGCAGAT |
| | TTACCACACTGCGACCCAGTAAAATCCCGTCTGG |
| | TCGCCCTTATTACTACCA (SEQ ID NO: 639) |
| otelixizumab-BtsI-20-3 | CCCTTTAATCAGATGCGTCGTGGTTCGTTAGTCGA |
| | TCTCCGCAGTGACTCCGTGAAGGGCAGATTTACC |
| | ATTTCCCGGGATAATAGCAAGAATACACTGTATCT |
| | GCAGATGAATTCACTGAGGGCTGAAGATACAGCC |
| | GTGTACACTGCGACCCAGTAAAATCCCGTCTGGT |
| | CGCCCTTATTACTACCA (SEQ ID NO: 640) |
| otelixizumab-BtsI-20-4 | CCCTTTAATCAGATGCGTCGTGGTTCGTTAGTCGA |
| | TCTCCGCAGTGGGGCTGAAGATACAGCCGTGTAT |
| | TATTGCGCCAAATTTCGCCAGTATTCTGGCGGCTT |
| | TGACTACTGGGGACAGGGCACTCTCGTCACAGT |
| | GAGCTCACTGCGACCCAGTAAAATCCCGTCTGGT |
| | CGCCCTTATTACTACCA (SEQ ID NO: 641) |
| otelixizumab-BtsI-20-5 | CCCTTTAATCAGATGCGTCGTGGTTCGTTAGTCGA |
| | TCTCCGCAGTGGGGCACTCTCGTCACAGTGAGCT |
| | CTGGCGGGTCCGGAGGCTCTGGCGGCGCCTCAG |
| | GCGCAGGCTCCGGAGGCGGCGACATTCAGCTCA |
| | CTCAACCCACTGCGACCCAGTAAAATCCCGTCTG |
| | GTCGCCCTTATTACTACCA (SEQ ID NO: 643) |
| otelixizumab-BtsI-20-6 | CCCTTTAATCAGATGCGTCGTGGTTCGTTAGTCGA |
| | TCTCCGCAGTGGGCGACATTCAGCTCACTCAACC |
| | CAACAGCGTGTCAACTTCTCTGGGATCCACCGTG |
| | AAGCTGTCCTGTACTCTCAGCTCTGGGAATATCGA |
| | AAATCACTGCGACCCAGTAAAATCCCGTCTGGTC |
| | GCCCTTATTACTACCA (SEQ ID NO: 644) |
| otelixizumab-BtsI-20-7 | CCCTTTAATCAGATGCGTCGTGGTTCGTTAGTCGA |
| | TCTCCGCAGTGCTCAGCTCTGGGAATATCGAAAA |
| | TAACTACGTGCATTGGTACCAGCTCTATGAGGGG |
| | CGGAGCCCCACTACCATGATTTATGACGACGATA |
| | AACGCCCCACTGCGACCCAGTAAAATCCCGTCTG |
| | GTCGCCCTTATTACTACCA (SEQ ID NO: 645) |
| otelixizumab-BtsI-20-8 | CCCTTTAATCAGATGCGTCGTGGTTCGTTAGTCGA |
| | TCTCCGCAGTGATGATTTATGACGACGATAAACGC |
| | CCTGACGGTGTGCCTGATAGATTTTCTGGCAGCAT |
| | CGATCGGTCTAGCAATAGCGCATTCCTGACTATCC |
| | ATCACTGCGACCCAGTAAAATCCCGTCTGGTCGCC |
| | CTTATTACTACCA (SEQ ID NO: 646) |
| otelixizumab-BtsI-20-9 | CCCTTTAATCAGATGCGTCGTGGTTCGTTAGTCGA |
| | TCTCCGCAGTGAATAGCGCATTCCTGACTATCCAT |
| | AATGTGGCAATCGAGGATGAGGCTATCTACTTCTG |
| | TCACTCCTATGTGAGCTCCTTCAACGTCTTCGGTG |
| | GCACTGCGACCCAGTAAAATCCCGTCTGGTCGCC |
| | CTTATTACTACCA (SEQ ID NO: 647) |
| otelixizumab-BtsI-20-10 | CCCTTTAATCAGATGCGTCGTGGTTCGTTAGTCGA |
| | TCTCCGCAGTGAGCTCCTTCAACGTCTTCGGTGGC |
| | GGCACAAAACTGACTGTTCTCGGGCCCGGCACCA |
| | GGTACATATCTCATTCACTGCGACCCAGTAAAATC |
| | CCGTCTGGTCGCCCTTATTACTACCA |
| | (SEQ ID NO: 648) |
| Panobacumab-BtsI-20-0 | CCCTTTAATCAGATGCGTCGTATTTTGTAGAGCGT |
| | TCGCGGCAGTGTTGAAGGGTGGATCATCGTACTG |
| | GCCCAGCCGGCCAGGCGCGAAGAACAGGTTGTT |
| | GAGTCAGGGGGCGGATTTGTGCAGCCTGGAGGA |
| | TCTCTGCACTGCCAAGACTTGCGAAGCAAAGAGG |
| | TCGCCCTTATTACTACCA (SEQ ID NO: 649) |
| Panobacumab-BtsI-20-1 | CCCTTTAATCAGATGCGTCGTATTTTGTAGAGCGT |
| | TCGCGGCAGTGGTGCAGCCTGGAGGATCTCTGAG |
| | ACTCAGCTGCGCAGCCAGCGGCTTCACCTTTTCA |
| | CCATACTGGATGCACTGGGTGAGACAAGCTCCTG |
| | GCCACTGCCAAGACTTGCGAAGCAAAGAGGTCG |
| | CCCTTATTACTACCA (SEQ ID NO: 650) |
| Panobacumab-BtsI-20-2 | CCCTTTAATCAGATGCGTCGTATTTTGTAGAGCGT |
| | TCGCGGCAGTGCTGGGTGAGACAAGCTCCTGGC |
| | AAGGGACTCGTCTGGGTGTCACGGATTAATTCTG |
| | ACGGATCAACATACTACGCAGACTCAGTCAAAGG |
| | AAGGTCACTGCCAAGACTTGCGAAGCAAAGAGG |
| | TCGCCCTTATTACTACCA (SEQ ID NO: 651) |
| Panobacumab-BtsI-20-3 | CCCTTTAATCAGATGCGTCGTATTTTGTAGAGCGT |
| | TCGCGGCAGTGACGCAGACTCAGTCAAAGGAAGG |
| | TTTACCATATCCAGAGATAACGCTAGAAACACACT |
| | GTATCTGCAGATGAACTCACTCAGAGCTGAGGAT |
| | ACAGCACTGCCAAGACTTGCGAAGCAAAGAGGTC |
| | GCCCTTATTACTACCA (SEQ ID NO: 652) |
| Panobacumab-BtsI-20-4 | CCCTTTAATCAGATGCGTCGTATTTTGTAGAGCGT |
| | TCGCGGCAGTGAACTCACTCAGAGCTGAGGATAC |
| | AGCAGTTTACTACTGTGCAAGAGACCGGTATTAT |
| | GGTCCTGAGATGTGGGGCCAGGGCACAATGGT |
| | GCACTGCCAAGACTTGCGAAGCAAAGAGGTCGC |
| | CCTTATTACTACCA (SEQ ID NO: 653) |
| Panobacumab-BtsI-20-5 | CCCTTTAATCAGATGCGTCGTATTTTGTAGAGCGT |
| | TCGCGGCAGTGGGGCCAGGGCACAATGGTGACC |
| | GTTAGCTCTGGCGGCGCAGGCTCTGGGGCTGGA |
| | TCAGGAAGCTCCGGTGCTGGTAGCGGCGATGTG |
| | GTGATGACACTGCCAAGACTTGCGAAGCAAAGA |
| | GGTCGCCCTTATTACTACCA (SEQ ID NO: 654) |
| Panobacumab-BtsI-20-6 | CCCTTTAATCAGATGCGTCGTATTTTGTAGAGCGT |
| | TCGCGGCAGTGTAGCGGCGATGTGGTGATGACC |
| | CAGTCTCCACTCAGCCTCCCCGTTACACTCGGGC |
| | AACCCGCCTCTATTTCTTGCCGCTCCTCCCAATCC |
| | CTCGCACTGCCAAGACTTGCGAAGCAAAGAGGT |
| | CGCCCTTATTACTACCA (SEQ ID NO: 655) |
| Panobacumab-BtsI-20-7 | CCCTTTAATCAGATGCGTCGTATTTTGTAGAGCGT |
| | TCGCGGCAGTGGCCGCTCCTCCCAATCCCTCGTG |
| | TACTCTGACGGCAATACATACCTGAATTGGTTCCA |
| | GCAGAGACCTGGGCAGTCACCAAGGAGACTCATT |
| | TACCACTGCCAAGACTTGCGAAGCAAAGAGGTCG |
| | CCCTTATTACTACCA (SEQ ID NO: 656) |
| Panobacumab-BtsI-20-8 | CCCTTTAATCAGATGCGTCGTATTTTGTAGAGCGT |
| | TCGCGGCAGTGCAGTCACCAAGGAGACTCATTTA |
| | CAAGGTGAGCAATCGCGACAGCGGGGTGCCCGA |
| | CCGGTTCAGCGGCAGCGGCTCAGGGACCGATTTT |
| | ACCCTCACTGCCAAGACTTGCGAAGCAAAGAGGT |
| | CGCCCTTATTACTACCA (SEQ ID NO: 657) |
| Panobacumab-BtsI-20-9 | CCCTTTAATCAGATGCGTCGTATTTTGTAGAGCGT |
| | TCGCGGCAGTGCGGCTCAGGGACCGATTTTACCC |
| | TCAAGATTTCAAGGGTGGAAGCTGAAGATGTGGG |
| | AGTCTATTATTGTATGCAGGGCACCCACTGGCCC |
| | ACTGCCAAGACTTGCGAAGCAAAGAGGTCGCCCT |
| | TATTACTACCA (SEQ ID NO: 658) |
| Panobacumab-BtsI-20-10 | CCCTTTAATCAGATGCGTCGTATTTTGTAGAGCGT |
| | TCGCGGCAGTGTGCAGGGCACCCACTGGCCCCT |
| | GACATTTGGCGGCGGGACAAAGGTCGAGATCAA |
| | GGGGCCCACAACGATAGGCCCAAGAATTTCACT |
| | GCCAAGACTTGCGAAGCAAAGAGGTCGCCCTTA |
| | TTACTACCA (SEQ ID NO: 659) |
| gantenerumab-BtsI-20-0 | CCCTTTAATCAGATGCGTCGTTCTGTAAGTTTCG |
| | TCGGGAGCAGTGTTGGCTGTTAGTTTTAGAGCC |
| | GGGCCCAGCCGGCCAGGCGCCAGGTCGAGCTG |
| | GTGGAGTCTGGCGGGGGGCTGGTGCAACCTGG |
| | GGGAAGCCTGCACTGCTAGTGAGGTGCGGTGTT |
| | TAGGGTCGCCCTTATTACTACCA |
| | (SEQ ID NO: 660) |
| gantenerumab-BtsI-20-1 | CCCTTTAATCAGATGCGTCGTTCTGTAAGTTTCG |
| | TCGGGAGCAGTGTGCAACCTGGGGGAAGCCTG |
| | AGGCTGTCCTGCGCTGCATCAGGGTTCACATTC |
| | TCTAGCTATGCAATGTCCTGGGTGAGGCAGGCC |
| | CCTGGAAAACACTGCTAGTGAGGTGCGGTGTTT |
| | AGGGTCGCCCTTATTACTACCA |
| | (SEQ ID NO: 661) |
| gantenerumab-BtsI-20-2 | CCCTTTAATCAGATGCGTCGTTCTGTAAGTTTCG |
| | TCGGGAGCAGTGAGGCAGGCCCCTGGAAAAGG |
| | ACTGGAGTGGGTCTCTGCAATCAATGCCTCTGG |
| | CACCCGCACTTATTATGCTGACAGCGTCAAGGG |
| | GAGGTTTACCACTGCTAGTGAGGTGCGGTGTTT |
| | AGGGTCGCCCTTATTACTACCA |
| | (SEQ ID NO: 662) |
| gantenerumab-BtsI-20-3 | CCCTTTAATCAGATGCGTCGTTCTGTAAGTTTCG |
| | TCGGGAGCAGTGCAGCGTCAAGGGGAGGTTTA |
| | CTATTTCTAGGGATAACTCTAAAAATACCCTGTA |
| | CCTCCAGATGAACTCACTCAGGGCCGAGGATAC |
| | TGCAGTTTCACTGCTAGTGAGGTGCGGTGTTTA |
| | GGGTCGCCCTTATTACTACCA (SEQ ID NO: 663) |
| gantenerumab-BtsI-20-4 | CCCTTTAATCAGATGCGTCGTTCTGTAAGTTTCG |
| | TCGGGAGCAGTGGGGCCGAGGATACTGCAGTT |
| | TACTATTGCGCTAGGGGTAAAGGTAACACCCAC |
| | AAGCCTTACGGATATGTGAGGTACTTCGACGTG |
| | TGGGGGCCACTGCTAGTGAGGTGCGGTGTTTAG |
| | GGTCGCCCTTATTACTACCA (SEQ ID NO: 664) |
| gantenerumab-BtsI-20-5 | CCCTTTAATCAGATGCGTCGTTCTGTAAGTTTCG |
| | TCGGGAGCAGTGAGGTACTTCGACGTGTGGGG |
| | GCAGGGAACCGGTGGCTCCGGCGGAAGCGGGG |
| | GAGCTTCCGGGGCTGGCTCTGGTGGGGGCGACA |
| | TCGTGCACTGCTAGTGAGGTGCGGTGTTTAGGG |
| | TCGCCCTTATTACTACCA (SEQ ID NO: 665) |
| gantenerumab-BtsI-20-6 | CCCTTTAATCAGATGCGTCGTTCTGTAAGTTTCG |
| | TCGGGAGCAGTGTGGTGGGGGCGACATCGTGC |
| | TCACCCAGTCCCCAGCCACTCTGAGCCTGAGCC |
| | CTGGAGAAAGAGCAACACTGTCTTGCCGGGCCT |
| | CCCAGTCCGCACTGCTAGTGAGGTGCGGTGTTT |
| | AGGGTCGCCCTTATTACTACCA (SEQ ID NO: 666) |
| gantenerumab-BtsI-20-7 | CCCTTTAATCAGATGCGTCGTTCTGTAAGTTTCGT |
| | CGGGAGCAGTGGCCGGGCCTCCCAGTCCGTTTC |
| | CAGCAGCTACCTGGCCTGGTATCAGCAGAAACCA |
| | GGCCAGGCACCAAGGCTCCTGATCTATGGTGCCT |
| | CTTCCCACTGCTAGTGAGGTGCGGTGTTTAGGGT |
| | CGCCCTTATTACTACCA (SEQ ID NO: 667) |
| gantenerumab-BtsI-20-8 | CCCTTTAATCAGATGCGTCGTTCTGTAAGTTTCGT |
| | CGGGAGCAGTGCTCCTGATCTATGGTGCCTCTTC |
| | CAGAGCAACCGGCGTGCCTGCTCGGTTCTCCGGG |
| | TCCGGCTCAGGGACCGACTTCACACTGACTATAT |
| | CCTCCACTGCTAGTGAGGTGCGGTGTTTAGGGTC |
| | GCCCTTATTACTACCA (SEQ ID NO: 668) |
| gantenerumab-BtsI-20-9 | CCCTTTAATCAGATGCGTCGTTCTGTAAGTTTCGT |
| | CGGGAGCAGTGACCGACTTCACACTGACTATATC |
| | CTCCCTGGAGCCAGAGGACTTTGCCACATACTAT |
| | TGTCTGCAAATCTACAATATGCCCATTACCTTTGG |
| | CCACACTGCTAGTGAGGTGCGGTGTTTAGGGTCG |
| | CCCTTATTACTACCA (SEQ ID NO: 669) |
| gantenerumab-BtsI-20-10 | CCCTTTAATCAGATGCGTCGTTCTGTAAGTTTCGT |
| | CGGGAGCAGTGCAATATGCCCATTACCTTTGGCC |
| | AGGGTACCAAAGTCGAGATCAAGGGGCCCACGA |
| | CGGCTGTATATGGTTTTTCACTGCTAGTGAGGTG |
| | CGGTGTTTAGGGTCGCCCTTATTACTACCA |
| | (SEQ ID NO: 670) |
| milatuzumab-BtsI-20-0 | CCCTTTAATCAGATGCGTCGTTGACGTACGTAGG |
| | TTCTCCGCAGTGTTAGTGGTGTAGTGGCTTCTAC |
| | GGCCCAGCCGGCCAGGCGCCAGGTCCAGCTGCA |
| | GCAGTCTGGATCCGAGCTCAAAAAGCCCGGAGC |
| | CAGCGCACTGCGCGTCAGTGTAGTTGTGTTCGGT |
| | CGCCCTTATTACTACCA (SEQ ID NO: 671) |
| milatuzumab-BtsI-20-1 | CCCTTTAATCAGATGCGTCGTTGACGTACGTAGG |
| | TTCTCCGCAGTGCAAAAAGCCCGGAGCCAGCGTT |
| | AAGGTTTCCTGCAAAGCCTCTGGCTATACCTTCAC |
| | TAATTACGGTGTGAACTGGATTAAGCAGGCCCCA |
| | GGCCCACTGCGCGTCAGTGTAGTTGTGTTCGGTC |
| | GCCCTTATTACTACCA (SEQ ID NO: 672) |
| milatuzumab-BtsI-20-2 | CCCTTTAATCAGATGCGTCGTTGACGTACGTAGG |
| | TTCTCCGCAGTGTGGATTAAGCAGGCCCCAGGC |
| | CAGGGGCTCCAATGGATGGGCTGGATAAACCCT |
| | AATACTGGAGAGCCTACTTTCGACGATGATTTCA |
| | AGGGGCGCCACTGCGCGTCAGTGTAGTTGTGTT |
| | CGGTCGCCCTTATTACTACCA (SEQ ID NO: 673) |
| milatuzumab-BtsI-20-3 | CCCTTTAATCAGATGCGTCGTTGACGTACGTAGG |
| | TTCTCCGCAGTGTCGACGATGATTTCAAGGGGCG |
| | CTTCGCCTTCTCTCTGGATACCTCCGTGTCAACTG |
| | CCTACCTCCAGATCTCAAGCCTGAAAGCCGACGA |
| | TACTGCCACTGCGCGTCAGTGTAGTTGTGTTCGG |
| | TCGCCCTTATTACTACCA (SEQ ID NO: 674) |
| milatuzumab-BtsI-20-4 | CCCTTTAATCAGATGCGTCGTTGACGTACGTAGG |
| | TTCTCCGCAGTGAGCCTGAAAGCCGACGATACTG |
| | CCGTGTACTTCTGTTCTAGGTCCAGAGGGAAGAA |
| | CGAGGCCTGGTTCGCATACTGGGGTCAGGGGAC |
| | ACTGGTGACACTGCGCGTCAGTGTAGTTGTGTTC |
| | GGTCGCCCTTATTACTACCA (SEQ ID NO: 675) |
| milatuzumab-BtsI-20-5 | CCCTTTAATCAGATGCGTCGTTGACGTACGTAGG |
| | TTCTCCGCAGTGGGGGTCAGGGGACACTGGTGA |
| | CTGTGAGCTCTGGAGGATCAGCAGGGTCAGGGT |
| | CTTCCGGCGGGGCTAGCGGCTCAGGGGGCGAC |
| | ATTCAGCTCACTGCGCGTCAGTGTAGTTGTGTTC |
| | GGTCGCCCTTATTACTACCA (SEQ ID NO: 676) |
| milatuzumab-BtsI-20-6 | CCCTTTAATCAGATGCGTCGTTGACGTACGTAGG |
| | TTCTCCGCAGTGCTCAGGGGGCGACATTCAGCTC |
| | ACCCAATCACCACTGTCTCTGCCCGTGACCCTCG |
| | GACAGCCCGCTTCAATCTCATGCCGGTCTTCTCA |
| | GTCACCACTGCGCGTCAGTGTAGTTGTGTTCGGT |
| | CGCCCTTATTACTACCA (SEQ ID NO: 677) |
| milatuzumab-BtsI-20-7 | CCCTTTAATCAGATGCGTCGTTGACGTACGTAGG |
| | TTCTCCGCAGTGTCATGCCGGTCTTCTCAGTCAC |
| | TCGTCCATCGGAACGGCAACACTTATCTGCACTG |
| | GTTTCAACAGCGGCCAGGCCAATCTCCCCGCCTG |
| | CTGCACTGCGCGTCAGTGTAGTTGTGTTCGGTCG |
| | CCCTTATTACTACCA (SEQ ID NO: 678) |
| milatuzumab-BtsI-20-8 | CCCTTTAATCAGATGCGTCGTTGACGTACGTAGG |
| | TTCTCCGCAGTGGCCAATCTCCCCGCCTGCTGAT |
| | TTACACTGTGAGCAATCGGTTCTCAGGTGTTCCT |
| | GACAGATTTAGCGGGAGCGGTAGCGGCACTGAT |
| | TTTACTCTCACTGCGCGTCAGTGTAGTTGTGTTC |
| | GGTCGCCCTTATTACTACCA (SEQ ID NO: 679) |
| milatuzumab-BtsI-20-9 | CCCTTTAATCAGATGCGTCGTTGACGTACGTAGG |
| | TTCTCCGCAGTGCGGTAGCGGCACTGATTTTACT |
| | CTGAAGATTTCCCGCGTCGAAGCCGAGGACGTC |
| | GGGGTGTACTTTTGCAGCCAGAGCTCTCATGTGC |
| | CCCCCCACTGCGCGTCAGTGTAGTTGTGTTCGG |
| | TCGCCCTTATTACTACCA (SEQ ID NO: 680) |
| milatuzumab-BtsI-20-10 | CCCTTTAATCAGATGCGTCGTTGACGTACGTAGG |
| | TTCTCCGCAGTGCAGAGCTCTCATGTGCCCCCCA |
| | CCTTCGGCGCAGGGACACGCCTGGAAATTAAGG |
| | GGCCCCATCGGGTGGGATTTAGCTATTCACTGCG |
| | CGTCAGTGTAGTTGTGTTCGGTCGCCCTTATTAC |
| | TACCA (SEQ ID NO: 681) |
| veltuzumab-BtsI-20-0 | CCCTTTAATCAGATGCGTCGGAGATGAGTAGACG |
| | AGTGGGGCAGTGTTCTCAGAGGGAGTTCAACTGT |
| | GGCCCAGCCGGCCAGGCGCCAGGTGCAGCTGCA |
| | GCAATCTGGCGCCGAAGTGAAAAAACCAGGTTCC |
| | TCCGTCCACTGCTAATGCGAGTCAGTGACCATGG |
| | TCGCCCTTATTACTACCA (SEQ ID NO: 682) |
| veltuzumab-BtsI-20-1 | CCCTTTAATCAGATGCGTCGGAGATGAGTAGACG |
| | AGTGGGGCAGTGGTGAAAAAACCAGGTTCCTCC |
| | GTCAAGGTGAGCTGCAAGGCCTCCGGCTACACCT |
| | TTACCTCATACAACATGCACTGGGTGAAACAAGC |
| | TCCTGGCACTGCTAATGCGAGTCAGTGACCATG |
| | GTCGCCCTTATTACTACCA (SEQ ID NO: 683) |
| veltuzumab-BtsI-20-2 | CCCTTTAATCAGATGCGTCGGAGATGAGTAGACG |
| | AGTGGGGCAGTGCACTGGGTGAAACAAGCTCCTG |
| | GTCAGGGCCTGGAGTGGATTGGCGCAATCTATCC |
| | CGGGAATGGCGACACTTCTTATAACCAAAAGTTC |
| | AAAGGCACTGCTAATGCGAGTCAGTGACCATGGT |
| | CGCCCTTATTACTACCA (SEQ ID NO: 684) |
| veltuzumab-BtsI-20-3 | CCCTTTAATCAGATGCGTCGGAGATGAGTAGACG |
| | AGTGGGGCAGTGCGACACTTCTTATAACCAAAAG |
| | TTCAAAGGAAAGGCCACACTCACAGCCGACGAAA |
| | GCACCAATACTGCCTACATGGAGCTGTCTAGCCT |
| | CCGCCACTGCTAATGCGAGTCAGTGACCATGGTC |
| | GCCCTTATTACTACCA (SEQ ID NO: 685) |
| veltuzumab-BtsI-20-4 | CCCTTTAATCAGATGCGTCGGAGATGAGTAGACG |
| | AGTGGGGCAGTGACATGGAGCTGTCTAGCCTCC |
| | GCTCTGAGGATACTGCCTTCTACTACTGTGCTCG |
| | GTCCACTTACTACGGGGGGGATTGGTACTTCGA |
| | TGTGTGGCACTGCTAATGCGAGTCAGTGACCAT |
| | GGTCGCCCTTATTACTACCA (SEQ ID NO: 686) |
| veltuzumab-BtsI-20-5 | CCCTTTAATCAGATGCGTCGGAGATGAGTAGACG |
| | AGTGGGGCAGTGGGGGATTGGTACTTCGATGTG |
| | TGGGGGCAAGGCACTACTGTCACAGTTTCTTCTG |
| | GGGGGGCCGGGAGCGGGGCCGGAAGCGGCAGC |
| | TCCACTGCTAATGCGAGTCAGTGACCATGGTCGC |
| | CCTTATTACTACCA (SEQ ID NO: 687) |
| veltuzumab-BtsI-20-6 | CCCTTTAATCAGATGCGTCGGAGATGAGTAGACG |
| | AGTGGGGCAGTGGGCCGGAAGCGGCAGCTCCGG |
| | CGCAGGCTCCGGGGATATCCAGCTGACACAGAG |
| | CCCTTCATCACTCTCCGCCTCTGTTGGAGATAGAG |
| | TCACAACACTGCTAATGCGAGTCAGTGACCATGG |
| | TCGCCCTTATTACTACCA (SEQ ID NO: 688) |
| veltuzumab-BtsI-20-7 | CCCTTTAATCAGATGCGTCGGAGATGAGTAGACG |
| | AGTGGGGCAGTGGCCTCTGTTGGAGATAGAGTC |
| | ACAATGACTTGTAGGGCCTCCTCTTCCGTGTCAT |
| | ACATCCACTGGTTCCAGCAGAAGCCCGGTAAGGC |
| | TCCACTGCTAATGCGAGTCAGTGACCATGGTCGC |
| | CCTTATTACTACCA (SEQ ID NO: 689) |
| veltuzumab-BtsI-20-8 | CCCTTTAATCAGATGCGTCGGAGATGAGTAGACG |
| | AGTGGGGCAGTGGCAGAAGCCCGGTAAGGCTCC |
| | CAAGCCTTGGATTTATGCCACATCCAATCTGGCCT |
| | CAGGTGTGCCCGTCCGCTTCTCCGGTAGCGGATC |
| | TGGGACCACTGCTAATGCGAGTCAGTGACCATGG |
| | TCGCCCTTATTACTACCA (SEQ ID NO: 690) |
| veltuzumab-BtsI-20-9 | CCCTTTAATCAGATGCGTCGGAGATGAGTAGACG |
| | AGTGGGGCAGTGTCCGGTAGCGGATCTGGGACT |
| | GATTATACTTTCACAATTAGCTCTCTGCAGCCAGA |
| | AGATATTGCAACTTACTATTGCCAACAGTGGACA |
| | TCCACACTGCTAATGCGAGTCAGTGACCATGGTC |
| | GCCCTTATTACTACCA (SEQ ID NO: 691) |
| veltuzumab-BtsI-20-10 | CCCTTTAATCAGATGCGTCGGAGATGAGTAGACG |
| | AGTGGGGCAGTGCTATTGCCAACAGTGGACATC |
| | CAATCCTCCTACTTTTGGAGGGGGGACTAAGCTC |
| | GAAATAAAGGGGCCCAGTCAAAACTGTAACCGC |
| | ACTTCACTGCTAATGCGAGTCAGTGACCATGGTC |
| | GCCCTTATTACTACCA (SEQ ID NO: 692) |
| Tanezumab-BtsI-20-0 | CCCTTTAATCAGATGCGTCGCTTTGGGCTTTCAGA |
| | TGAGCGCAGTGTTTTTGGCAGATCATTAACGGCG |
| | GCCCAGCCGGCCAGGCGCCAGGTTCAGCTCCAA |
| | GAGTCAGGTCCTGGGCTGGTTAAGCCTTCTGAGA |
| | CACTGCACTGCCCGACCGACAGAAATCTTTGGGT |
| | CGCCCTTATTACTACCA (SEQ ID NO: 693) |
| Tanezumab-BtsI-20-1 | CCCTTTAATCAGATGCGTCGCTTTGGGCTTTCAGA |
| | TGAGCGCAGTGCTGGTTAAGCCTTCTGAGACACT |
| | GAGCCTGACCTGCACCGTTAGCGGCTTCTCCCTG |
| | ATCGGCTACGATCTGAACTGGATTCGGCAGCCAC |
| | CACTGCCCGACCGACAGAAATCTTTGGGTCGCCC |
| | TTATTACTACCA (SEQ ID NO: 694) |
| Tanezumab-BtsI-20-2 | CCCTTTAATCAGATGCGTCGCTTTGGGCTTTCAGA |
| | TGAGCGCAGTGGAACTGGATTCGGCAGCCACCCG |
| | GAAAGGGCCTGGAATGGATTGGCATAATCTGGGG |
| | AGACGGGACAACTGACTATAATTCTGCCGTTAAGT |
| | CACGCGCACTGCCCGACCGACAGAAATCTTTGGG |
| | TCGCCCTTATTACTACCA (SEQ ID NO: 695) |
| Tanezumab-BtsI-20-3 | CCCTTTAATCAGATGCGTCGCTTTGGGCTTTCAGA |
| | TGAGCGCAGTGACTATAATTCTGCCGTTAAGTCAC |
| | GCGTGACCATATCTAAAGACACAAGCAAGAACCA |
| | GTTCAGCCTGAAACTGTCCTCAGTCACAGCAGCA |
| | GCACTGCCCGACCGACAGAAATCTTTGGGTCGCC |
| | CTTATTACTACCA (SEQ ID NO: 696) |
| Tanezumab-BtsI-20-4 | CCCTTTAATCAGATGCGTCGCTTTGGGCTTTCAGA |
| | TGAGCGCAGTGCTGTCCTCAGTCACAGCAGCAGA |
| | TACTGCTGTGTATTACTGTGCCCGCGGGGGCTAT |
| | TGGTACGCTACCTCATATTACTTTGATTACTGGGG |
| | GCAGCACTGCCCGACCGACAGAAATCTTTGGGTC |
| | GCCCTTATTACTACCA (SEQ ID NO: 697) |
| Tanezumab-BtsI-20-5 | CCCTTTAATCAGATGCGTCGCTTTGGGCTTTCAGA |
| | TGAGCGCAGTGATATTACTTTGATTACTGGGGGC |
| | AGGGCACCCTGGTGACCGTCTCCTCTGGAGGCTC |
| | TGGTGGGTCTGGAGGAGCATCTGGGGCCGGGACA |
| | CTGCCCGACCGACAGAAATCTTTGGGTCGCCCTTA |
| | TTACTACCA (SEQ ID NO: 698) |
| Tanezumab-BtsI-20-6 | CCCTTTAATCAGATGCGTCGCTTTGGGCTTTCAGA |
| | TGAGCGCAGTGGAGCATCTGGGGCCGGGAGCGG |
| | CGGGGGGGATATTCAGATGACTCAATCACCCTCA |
| | AGCCTCTCAGCCTCAGTCGGGGACCGGGTGACAA |
| | TCACCCACTGCCCGACCGACAGAAATCTTTGGGTC |
| | GCCCTTATTACTACCA (SEQ ID NO: 699) |
| Tanezumab-BtsI-20-7 | CCCTTTAATCAGATGCGTCGCTTTGGGCTTTCAGA |
| | TGAGCGCAGTGGGGGACCGGGTGACAATCACCT |
| | GTAGGGCTTCACAAAGCATATCCAACAATCTGAAT |
| | TGGTACCAGCAAAAACCAGGAAAAGCCCCAAAAC |
| | TCCTCACTGCCCGACCGACAGAAATCTTTGGGTC |
| | GCCCTTATTACTACCA (SEQ ID NO: 700) |
| Tanezumab-BtsI-20-8 | CCCTTTAATCAGATGCGTCGCTTTGGGCTTTCAGA |
| | TGAGCGCAGTGACCAGGAAAAGCCCCAAAACTCC |
| | TGATATACTATACCTCCCGGTTCCACAGCGGGGT |
| | GCCTAGCAGGTTCAGCGGCTCCGGCAGCGGCAC |
| | TGATTCACTGCCCGACCGACAGAAATCTTTGGGT |
| | CGCCCTTATTACTACCA (SEQ ID NO: 701) |
| Tanezumab-BtsI-20-9 | CCCTTTAATCAGATGCGTCGCTTTGGGCTTTCAGA |
| | TGAGCGCAGTGCCGGCAGCGGCACTGATTTCACT |
| | TTCACCATTTCCTCCCTGCAACCAGAGGACATTGC |
| | AACTTATTATTGCCAGCAGGAGCATACCCTGCCAT |
| | ATCACTGCCCGACCGACAGAAATCTTTGGGTCGC |
| | CCTTATTACTACCA (SEQ ID NO: 702) |
| Tanezumab-BtsI-20-10 | CCCTTTAATCAGATGCGTCGCTTTGGGCTTTCAGA |
| | TGAGCGCAGTGGCAGGAGCATACCCTGCCATATA |
| | CTTTCGGCCAGGGTACAAAGCTGGAGATAAAGGG |
| | GCCCCTGTCACCCTATGTAGTCCCTTCACTGCCCG |
| | ACCGACAGAAATCTTTGGGTCGCCCTTATTACTAC |
| | CA (SEQ ID NO: 703) |
| anrukinzumab-BtsI-20-0 | CCCTTTAATCAGATGCGTCGTGTCATATGCTAACG |
| | TCCGTGCAGTGTTTATGATCTCCGTACACGAGCGG |
| | CCCAGCCGGCCAGGCGCGAAGTGCAACTGGTCG |
| | AAAGCGGGGGTGGACTGGTGCAGCCTGGGGGCA |
| | CACTGCTTCCGCTAAGAAAGTAGCCAGGTCGCCC |
| | TTATTACTACCA (SEQ ID NO: 704) |
| anrukinzumab-BtsI-20-1 | CCCTTTAATCAGATGCGTCGTGTCATATGCTAACG |
| | TCCGTGCAGTGTGGTGCAGCCTGGGGGCAGCCT |
| | GCGCCTGAGCTGTGCAGCTTCAGGCTTTACCTTC |
| | ATCAGCTACGCTATGTCTTGGGTGAGACAGGCCC |
| | CCCACTGCTTCCGCTAAGAAAGTAGCCAGGTCGC |
| | CCTTATTACTACCA (SEQ ID NO: 705) |
| anrukinzumab-BtsI-20-2 | CCCTTTAATCAGATGCGTCGTGTCATATGCTAACG |
| | TCCGTGCAGTGCTTGGGTGAGACAGGCCCCCGG |
| | AAAAGGACTCGAATGGGTGGCTAGCATCTCAAGC |
| | GGTGGCAATACATACTACCCCGACAGCGTCAAGG |
| | GCCGGTCACTGCTTCCGCTAAGAAAGTAGCCAGG |
| | TCGCCCTTATTACTACCA (SEQ ID NO: 706) |
| anrukinzumab-BtsI-20-3 | CCCTTTAATCAGATGCGTCGTGTCATATGCTAACG |
| | TCCGTGCAGTGACAGCGTCAAGGGCCGGTTTACC |
| | ATCTCACGCGACAATGCCAAGAATTCCCTGTACCT |
| | GCAGATGAACTCCCTGCGCGCTGAAGATACAGCC |
| | GTCTCACTGCTTCCGCTAAGAAAGTAGCCAGGTCG |
| | CCCTTATTACTACCA (SEQ ID NO: 707) |
| anrukinzumab-BtsI-20-4 | CCCTTTAATCAGATGCGTCGTGTCATATGCTAACG |
| | TCCGTGCAGTGCGCGCTGAAGATACAGCCGTCTA |
| | TTATTGCGCTCGGCTGGACGGCTACTACTTTGGCT |
| | TCGCATACTGGGGCCAGGGGACCCTGGTGACAGT |
| | CAGCCACTGCTTCCGCTAAGAAAGTAGCCAGGTC |
| | GCCCTTATTACTACCA (SEQ ID NO: 708) |
| anrukinzumab-BtsI-20-5 | CCCTTTAATCAGATGCGTCGTGTCATATGCTAACG |
| | TCCGTGCAGTGGGGACCCTGGTGACAGTCAGCTC |
| | CGGGGGGAGCGCCGGCTCAGGGTCCTCCGGTGG |
| | TGCCTCTGGCTCAGGGGGGGACATTCAAATGACA |
| | CAGAGCCACTGCTTCCGCTAAGAAAGTAGCCAGG |
| | TCGCCCTTATTACTACCA (SEQ ID NO: 709) |
| anrukinzumab-BtsI-20-6 | CCCTTTAATCAGATGCGTCGTGTCATATGCTAACG |
| | TCCGTGCAGTGGGGGGACATTCAAATGACACAGA |
| | GCCCCTCTTCTCTCTCAGCTAGCGTGGGCGACCGC |
| | GTTACAATTACTTGCAAAGCCAGCGAATCCGTCGA |
| | TAACACTGCTTCCGCTAAGAAAGTAGCCAGGTCGC |
| | CCTTATTACTACCA (SEQ ID NO: 710) |
| anrukinzumab-BtsI-20-7 | CCCTTTAATCAGATGCGTCGTGTCATATGCTAACG |
| | TCCGTGCAGTGAGCCAGCGAATCCGTCGATAACT |
| | ATGGGAAGTCCCTGATGCACTGGTATCAACAGAA |
| | ACCTGGAAAGGCTCCCAAACTGCTCATCTACCGG |
| | GCTCACTGCTTCCGCTAAGAAAGTAGCCAGGTCG |
| | CCCTTATTACTACCA (SEQ ID NO: 711) |
| anrukinzumab-BtsI-20-8 | CCCTTTAATCAGATGCGTCGTGTCATATGCTAACG |
| | TCCGTGCAGTGCAAACTGCTCATCTACCGGGCTT |
| | CAAACCTGGAGAGCGGTGTGCCCTCACGGTTCTC |
| | CGGATCTGGAAGCGGGACTGACTTTACCCTCACC |
| | ATCTCCACTGCTTCCGCTAAGAAAGTAGCCAGGT |
| | CGCCCTTATTACTACCA (SEQ ID NO: 712) |
| anrukinzumab-BtsI-20-9 | CCCTTTAATCAGATGCGTCGTGTCATATGCTAACG |
| | TCCGTGCAGTGGACTGACTTTACCCTCACCATCTC |
| | CTCACTCCAACCAGAGGATTTCGCTACATATTATT |
| | GCCAGCAATCTAACGAGGATCCATGGACATTCGG |
| | GGCACTGCTTCCGCTAAGAAAGTAGCCAGGTCGC |
| | CCTTATTACTACCA (SEQ ID NO: 713) |
| anrukinzumab-BtsI-20-10 | CCCTTTAATCAGATGCGTCGTGTCATATGCTAACG |
| | TCCGTGCAGTGCGAGGATCCATGGACATTCGGGG |
| | GGGGCACAAAGGTTGAAATCAAGGGGCCCACTTC |
| | TTTGGAACGACAACGTTCACTGCTTCCGCTAAGAA |
| | AGTAGCCAGGTCGCCCTTATTACTACCA |
| | (SEQ ID NO: 714) |
| ustekinumab-BtsI-20-0 | CCCTTTAATCAGATGCGTCGTTGCGACATCACAAT |
| | TCTCGGCAGTGTTAGTGCCATGTTATCCCTGAAGG |
| | CCCAGCCGGCCAGGCGCGAGGTGCAACTCGTCCA |
| | GAGCGGCGCCGAGGTTAAGAAGCCTGGCGAGTCC |
| | CCACTGCACGCATGAAGTCTCGAAGTAGGTCGCCC |
| | TTATTACTACCA (SEQ ID NO: 715) |
| ustekinumab-BtsI-20-1 | CCCTTTAATCAGATGCGTCGTTGCGACATCACAAT |
| | TCTCGGCAGTGGTTAAGAAGCCTGGCGAGTCCCT |
| | GAAAATTTCCTGCAAAGGCAGCGGGTACTCTTTCA |
| | CTACATACTGGCTGGGTTGGGTGCGGCAGATGCC |
| | ACTGCACGCATGAAGTCTCGAAGTAGGTCGCCCT |
| | TATTACTACCA (SEQ ID NO: 716) |
| ustekinumab-BtsI-20-2 | CCCTTTAATCAGATGCGTCGTTGCGACATCACAAT |
| | TCTCGGCAGTGGGGTTGGGTGCGGCAGATGCCCG |
| | GGAAGGGGCTGGATTGGATCGGCATAATGTCCCC |
| | AGTGGATTCAGACATACGCTATAGCCCCTCCTTCC |
| | AGGCACTGCACGCATGAAGTCTCGAAGTAGGTCG |
| | CCCTTATTACTACCA (SEQ ID NO: 717) |
| ustekinumab-BtsI-20-3 | CCCTTTAATCAGATGCGTCGTTGCGACATCACAAT |
| | TCTCGGCAGTGACGCTATAGCCCCTCCTTCCAGGG |
| | TCAGGTGACCATGAGCGTCGATAAGAGCATTACT |
| | ACCGCCTACCTCCAGTGGAATTCCCTGAAGGCCT |
| | CTGCACTGCACGCATGAAGTCTCGAAGTAGGTCG |
| | CCCTTATTACTACCA (SEQ ID NO: 718) |
| ustekinumab-BtsI-20-4 | CCCTTTAATCAGATGCGTCGTTGCGACATCACAAT |
| | TCTCGGCAGTGGTGGAATTCCCTGAAGGCCTCTG |
| | ATACAGCCATGTACTACTGCGCCCGCAGACGCCC |
| | AGGACAGGGATACTTCGACTTCTGGGGCCAGGGA |
| | CACTGCACGCATGAAGTCTCGAAGTAGGTCGCCC |
| | TTATTACTACCA (SEQ ID NO: 719) |
| ustekinumab-BtsI-20-5 | CCCTTTAATCAGATGCGTCGTTGCGACATCACAAT |
| | TCTCGGCAGTGTCGACTTCTGGGGCCAGGGAACC |
| | CTCGTGACCGTTTCAAGCGGCGGGGCAGGGTCTG |
| | GCGCAGGAAGCGGCAGCAGCGGAGCCGGATCTG |
| | CACTGCACGCATGAAGTCTCGAAGTAGGTCGCCC |
| | TTATTACTACCA (SEQ ID NO: 720) |
| ustekinumab-BtsI-20-6 | CCCTTTAATCAGATGCGTCGTTGCGACATCACAAT |
| | TCTCGGCAGTGAGCAGCGGAGCCGGATCTGGGGA |
| | TATTCAGATGACCCAGTCTCCTTCTTCCCTCTCTG |
| | CTAGCGTCGGCGATAGGGTTACAATCACTTGCAG |
| | GGCCACTGCACGCATGAAGTCTCGAAGTAGGTCG |
| | CCCTTATTACTACCA (SEQ ID NO: 721) |
| ustekinumab-BtsI-20-7 | CCCTTTAATCAGATGCGTCGTTGCGACATCACAAT |
| | TCTCGGCAGTGTAGGGTTACAATCACTTGCAGGG |
| | CCAGCCAGGGCATATCATCTTGGCTGGCTTGGTA |
| | TCAGCAGAAGCCAGAAAAGGCCCCTAAGAGCCTC |
| | ATATCACTGCACGCATGAAGTCTCGAAGTAGGTC |
| | GCCCTTATTACTACCA (SEQ ID NO: 722) |
| ustekinumab-BtsI-20-8 | CCCTTTAATCAGATGCGTCGTTGCGACATCACAAT |
| | TCTCGGCAGTGAAGGCCCCTAAGAGCCTCATATAT |
| | GCTGCCAGCTCCCTGCAGTCCGGCGTGCCCTCCC |
| | GCTTCTCAGGCTCAGGTTCAGGGACAGACTTCAC |
| | ACTCACTGCACGCATGAAGTCTCGAAGTAGGTCG |
| | CCCTTATTACTACCA (SEQ ID NO: 723) |
| ustekinumab-BtsI-20-9 | CCCTTTAATCAGATGCGTCGTTGCGACATCACAAT |
| | TCTCGGCAGTGAGGTTCAGGGACAGACTTCACAC |
| | TGACAATCTCCTCCCTCCAGCCAGAGGATTTCGCC |
| | ACCTATTATTGCCAACAGTACAATATCTACCCTTA |
| | CACCTTCACTGCACGCATGAAGTCTCGAAGTAGG |
| | TCGCCCTTATTACTACCA (SEQ ID NO: 724) |
| ustekinumab-BtsI-20-10 | CCCTTTAATCAGATGCGTCGTTGCGACATCACAAT |
| | TCTCGGCAGTGAACAGTACAATATCTACCCTTACA |
| | CCTTTGGCCAGGGCACCAAACTGGAAATCAAGGG |
| | GCCCGGGTCCGTATATGTGTGACTTTCACTGCACG |
| | CATGAAGTCTCGAAGTAGGTCGCCCTTATTACTAC |
| | CA (SEQ ID NO: 725) |
| dacetuzumab-BtsI-20-0 | CCCTTTAATCAGATGCGTCGTCAGTATGGCGTCTT |
| | GAAGTGCAGTGTTTTATACATCTGGACGCCTCCGG |
| | CCCAGCCGGCCAGGCGCGAAGTGCAACTGGTGGA |
| | GTCTGGGGGAGGCCTGGTTCAGCCCGGTGGGACA |
| | CTGCCATAATAGAGGTCGGGCCATGGTCGCCCTTA |
| | TTACTACCA (SEQ ID NO: 726) |
| dacetuzumab-BtsI-20-1 | CCCTTTAATCAGATGCGTCGTCAGTATGGCGTCTT |
| | GAAGTGCAGTGCTGGTTCAGCCCGGTGGGAGCCT |
| | GCGGCTGTCCTGCGCCGCTTCCGGCTACTCATTC |
| | ACCGGATACTACATCCATTGGGTGAGGCAGGCCC |
| | CACTGCCATAATAGAGGTCGGGCCATGGTCGCCC |
| | TTATTACTACCA (SEQ ID NO: 727) |
| dacetuzumab-BtsI-20-2 | CCCTTTAATCAGATGCGTCGTCAGTATGGCGTCTT |
| | GAAGTGCAGTGCCATTGGGTGAGGCAGGCCCCTG |
| | GGAAGGGCCTGGAATGGGTGGCTAGAGTCATTCC |
| | TAATGCCGGTGGAACAAGCTACAATCAGAAATTCA |
| | AGGGGCCACTGCCATAATAGAGGTCGGGCCATGG |
| | TCGCCCTTATTACTACCA (SEQ ID NO: 728) |
| dacetuzumab-BtsI-20-3 | CCCTTTAATCAGATGCGTCGTCAGTATGGCGTCTT |
| | GAAGTGCAGTGCAAGCTACAATCAGAAATTCAAG |
| | GGGCGGTTTACCCTGAGCGTTGACAACTCTAAGA |
| | ATACTGCATATCTGCAGATGAACTCTCTGCGGGCC |
| | GCACTGCCATAATAGAGGTCGGGCCATGGTCGCC |
| | CTTATTACTACCA (SEQ ID NO: 729) |
| dacetuzumab-BtsI-20-4 | CCCTTTAATCAGATGCGTCGTCAGTATGGCGTCTT |
| | GAAGTGCAGTGCAGATGAACTCTCTGCGGGCCGA |
| | GGACACCGCCGTGTATTACTGCGCCAGGGAAGGA |
| | ATCTATTGGTGGGGCCAAGGTACCCTGGTGACAG |
| | TCTCACTGCCATAATAGAGGTCGGGCCATGGTCG |
| | CCCTTATTACTACCA (SEQ ID NO: 730) |
| dacetuzumab-BtsI-20-5 | CCCTTTAATCAGATGCGTCGTCAGTATGGCGTCTT |
| | GAAGTGCAGTGCCAAGGTACCCTGGTGACAGTCT |
| | CTTCCGGGGGCTCAGGAGGATCTGGAGGTGCATC |
| | CGGCGCCGGAAGCGGAGGGGGCGACATCCAGAT |
| | GACACCACTGCCATAATAGAGGTCGGGCCATGGT |
| | CGCCCTTATTACTACCA (SEQ ID NO: 731) |
| dacetuzumab-BtsI-20-6 | CCCTTTAATCAGATGCGTCGTCAGTATGGCGTCTT |
| | GAAGTGCAGTGGGGGGCGACATCCAGATGACACA |
| | GTCCCCTTCTTCTCTCTCTGCATCCGTTGGAGATA |
| | GAGTTACAATTACTTGTCGGAGCTCTCAGTCACTG |
| | GTCACTGCCATAATAGAGGTCGGGCCATGGTCGC |
| | CCTTATTACTACCA (SEQ ID NO: 732) |
| dacetuzumab-BtsI-20-7 | CCCTTTAATCAGATGCGTCGTCAGTATGGCGTCTT |
| | GAAGTGCAGTGGTCGGAGCTCTCAGTCACTGGTG |
| | CACAGCAACGGTAACACATTCCTGCACTGGTACCA |
| | GCAGAAACCTGGCAAAGCCCCTAAGCTGCTGATA |
| | TACCACTGCCATAATAGAGGTCGGGCCATGGTCG |
| | CCCTTATTACTACCA (SEQ ID NO: 733) |
| dacetuzumab-BtsI-20-8 | CCCTTTAATCAGATGCGTCGTCAGTATGGCGTCTT |
| | GAAGTGCAGTGAAAGCCCCTAAGCTGCTGATATA |
| | CACAGTCTCCAACCGGTTCTCTGGAGTGCCCTCCA |
| | GGTTTTCAGGAAGCGGGTCAGGGACAGACTTTAC |
| | CCCACTGCCATAATAGAGGTCGGGCCATGGTCGC |
| | CCTTATTACTACCA (SEQ ID NO: 734) |
| dacetuzumab-BtsI-20-9 | CCCTTTAATCAGATGCGTCGTCAGTATGGCGTCTT |
| | GAAGTGCAGTGCGGGTCAGGGACAGACTTTACCC |
| | TGACTATCTCCTCTCTGCAACCTGAGGATTTCGCC |
| | ACCTATTTCTGCAGCCAAACTACCCATGTTCCCTG |
| | GCACTGCCATAATAGAGGTCGGGCCATGGTCGCC |
| | CTTATTACTACCA (SEQ ID NO: 735) |
| dacetuzumab-BtsI-20-10 | CCCTTTAATCAGATGCGTCGTCAGTATGGCGTCTT |
| | GAAGTGCAGTGGCCAAACTACCCATGTTCCCTGG |
| | ACTTTTGGTCAGGGGACCAAGGTTGAGATCAAGG |
| | GGCCCCGCCATAATAGGGGTTCTCTTTCACTGCCA |
| | TAATAGAGGTCGGGCCATGGTCGCCCTTATTACT |
| | ACCA (SEQ ID NO: 736) |
| Alacizumab-BtsI-20-0 | CCCTTTAATCAGATGCGTCGTCATGTCGTGACCAG |
| | TAGACGCAGTGTTTCCTCGATTCTCCAATCAGGGG |
| | CCCAGCCGGCCAGGCGCGAAGTCCAACTCGTGGA |
| | GTCCGGGGGAGGCCTGGTGCAGCCCGGTGGGAG |
| | CCTGAGGCTCCACTGCGACGAAGTTCACTAGACCC |
| | AGGTCGCCCTTATTACTACCA (SEQ ID NO: 737) |
| Alacizumab-BtsI-20-1 | CCCTTTAATCAGATGCGTCGTCATGTCGTGACCAG |
| | TAGACGCAGTGCGGTGGGAGCCTGAGGCTCTCCT |
| | GTGCCGCCAGCGGCTTCACATTCTCTTCCTACGGT |
| | ATGTCATGGGTCAGGCAGGCCCCCGGAAAAGGCC |
| | TGGAATGGGCACTGCGACGAAGTTCACTAGACCC |
| | AGGTCGCCCTTATTACTACCA (SEQ ID NO: 738) |
| Alacizumab-BtsI-20-2 | CCCTTTAATCAGATGCGTCGTCATGTCGTGACCAG |
| | TAGACGCAGTGCCCGGAAAAGGCCTGGAATGGGT |
| | CGCAACCATAACATCCGGCGGCAGCTATACATACT |
| | ACGTGGATAGCGTTAAGGGGAGGTTCACAATTTC |
| | CCGGGACACACTGCGACGAAGTTCACTAGACCCA |
| | GGTCGCCCTTATTACTACCA (SEQ ID NO: 739) |
| Alacizumab-BtsI-20-3 | CCCTTTAATCAGATGCGTCGTCATGTCGTGACCAG |
| | TAGACGCAGTGGAGGTTCACAATTTCCCGGGACA |
| | ACGCCAAAAACACACTGTACCTGCAGATGAACTC |
| | TCTGCGGGCCGAGGATACCGCTGTGTACTATTGC |
| | GTGAGGATAGCACTGCGACGAAGTTCACTAGACC |
| | CAGGTCGCCCTTATTACTACCA (SEQ ID NO: 740) |
| Alacizumab-BtsI-20-4 | CCCTTTAATCAGATGCGTCGTCATGTCGTGACCAG |
| | TAGACGCAGTGCTGTGTACTATTGCGTGAGGATA |
| | GGCGAAGATGCTCTGGACTACTGGGGACAGGGG |
| | ACTCTGGTCACAGTGTCAAGCGGCGGCAGCGCC |
| | GGCTCAGGTAGCCACTGCGACGAAGTTCACTAGA |
| | CCCAGGTCGCCCTTATTACTACCA |
| | (SEQ ID NO: 741) |
| Alacizumab-BtsI-20-5 | CCCTTTAATCAGATGCGTCGTCATGTCGTGACCA |
| | GTAGACGCAGTGAGCGCCGGCTCAGGTAGCTCT |
| | GGGGGTGCCTCTGGATCCGGCGGCGATATCCAG |
| | ATGACACAATCTCCTTCCAGCCTGTCCGCCTCCG |
| | TGGGTGACAGGGTCACTGCGACGAAGTTCACTA |
| | GACCCAGGTCGCCCTTATTACTACCA |
| | (SEQ ID NO: 742) |
| Alacizumab-BtsI-20-6 | CCCTTTAATCAGATGCGTCGTCATGTCGTGACCA |
| | GTAGACGCAGTGGCCTCCGTGGGTGACAGGGTG |
| | ACCATTACATGTAGAGCATCACAGGACATCGCAG |
| | GGTCCCTGAATTGGCTGCAACAAAAGCCTGGGA |
| | AAGCTATCAAAAGCACTGCGACGAAGTTCACTAG |
| | ACCCAGGTCGCCCTTATTACTACCA |
| | (SEQ ID NO: 743) |
| Alacizumab-BtsI-20-7 | CCCTTTAATCAGATGCGTCGTCATGTCGTGACCA |
| | GTAGACGCAGTGAAAGCCTGGGAAAGCTATCAA |
| | AAGGCTGATTTACGCAACAAGCTCTCTCGACAGC |
| | GGCGTTCCTAAGAGATTCTCTGGCTCTAGGTCAG |
| | GAAGCGATTATACACTGCGACGAAGTTCACTAGA |
| | CCCAGGTCGCCCTTATTACTACCA |
| | (SEQ ID NO: 744) |
| Alacizumab-BtsI-20-8 | CCCTTTAATCAGATGCGTCGTCATGTCGTGACC |
| | AGTAGACGCAGTGGCTCTAGGTCAGGAAGCGA |
| | TTATACCCTGACTATCTCTAGCCTCCAGCCTGA |
| | AGATTTTGCCACTTATTATTGCCTCCAGTACGGG |
| | TCTTTCCCACCTACACTGCGACGAAGTTCACTAG |
| | ACCCAGGTCGCCCTTATTACTACCA |
| | (SEQ ID NO: 745) |
| Alacizumab-BtsI-20-9 | CCCTTTAATCAGATGCGTCGTCATGTCGTGACC |
| | AGTAGACGCAGTGCAGTACGGGTCTTTCCCACC |
| | TACCTTTGGTCAGGGCACAAAAGTCGAGATAAA |
| | AGGGCCCCGCATGTTTTAGCCTAACGATTCACT |
| | GCGACGAAGTTCACTAGACCCAGGTCGCCCTTA |
| | TTACTACCA |
| | (SEQ ID NO: 746) |
| tigatuzumab-BtsI-20-0 | CCCTTTAATCAGATGCGTCGAACTAACGGATTT |
| | AAGCGCGGCAGTGTTGCTTAACGCATTTCAAGC |
| | ACGGCCCAGCCGGCCAGGCGCGAAGTTCAGCT |
| | GGTGGAGTCCGGGGGGGGTCTGGTCCAGCCAG |
| | GAGGTTCACTCCACTGCCGGACGAAGCAACATA |
| | TGTTGGTCGCCCTTATTACTACCA |
| | (SEQ ID NO: 747) |
| tigatuzumab-BtsI-20-1 | CCCTTTAATCAGATGCGTCGAACTAACGGATTT |
| | AAGCGCGGCAGTGGTCCAGCCAGGAGGTTCAC |
| | TCCGCCTCTCTTGCGCAGCCTCAGGCTTCACCT |
| | TTAGCTCTTACGTGATGTCCTGGGTCAGGCAGG |
| | CCCCACTGCCGGACGAAGCAACATATGTTGGTC |
| | GCCCTTATTACTACCA (SEQ ID NO: 748) |
| tigatuzumab-BtsI-20-2 | CCCTTTAATCAGATGCGTCGAACTAACGGATTT |
| | AAGCGCGGCAGTGCCTGGGTCAGGCAGGCCCC |
| | TGGCAAGGGTCTCGAATGGGTTGCCACAATCT |
| | CTTCAGGCGGAAGCTACACCTACTATCCCGAC |
| | TCTGTTAAAGGAACACTGCCGGACGAAGCAAC |
| | ATATGTTGGTCGCCCTTATTACTACCA |
| | (SEQ ID NO: 749) |
| tigatuzumab-BtsI-20-3 | CCCTTTAATCAGATGCGTCGAACTAACGGATTT |
| | AAGCGCGGCAGTGTACTATCCCGACTCTGTTA |
| | AAGGAAGATTCACAATTTCCAGAGATAACGCCA |
| | AAAACACACTGTACCTGCAAATGAATTCACTGA |
| | GAGCTGAGGACACTGCCGGACGAAGCAACATA |
| | TGTTGGTCGCCCTTATTACTACCA |
| | (SEQ ID NO: 750) |
| tigatuzumab-BtsI-20-4 | CCCTTTAATCAGATGCGTCGAACTAACGGATTT |
| | AAGCGCGGCAGTGAATGAATTCACTGAGAGCT |
| | GAGGATACTGCTGTGTACTACTGCGCCAGACG |
| | CGGTGACTCCATGATCACCACCGACTATTGGG |
| | GTCAGGGGACTCACTGCCGGACGAAGCAACAT |
| | ATGTTGGTCGCCCTTATTACTACCA |
| | (SEQ ID NO: 751) |
| tigatuzumab-BtsI-20-5 | CCCTTTAATCAGATGCGTCGAACTAACGGATTT |
| | AAGCGCGGCAGTGCCGACTATTGGGGTCAGGG |
| | GACTCTGGTCACCGTGTCATCCGGGGGAGCCG |
| | GGAGCGGGGCTGGCAGCGGATCTTCTGGAGCA |
| | GGTTCTGGCGCACTGCCGGACGAAGCAACATA |
| | TGTTGGTCGCCCTTATTACTACCA |
| | (SEQ ID NO: 752) |
| tigatuzumab-BtsI-20-6 | CCCTTTAATCAGATGCGTCGAACTAACGGATTT |
| | AAGCGCGGCAGTGTCTTCTGGAGCAGGTTCTG |
| | GCGACATCCAGATGACACAAAGCCCTTCATCCC |
| | TCTCTGCATCTGTCGGCGATCGCGTGACTATAA |
| | CCTGCAAAGCCACTGCCGGACGAAGCAACATA |
| | TGTTGGTCGCCCTTATTACTACCA |
| | (SEQ ID NO: 753) |
| tigatuzumab-BtsI-20-7 | CCCTTTAATCAGATGCGTCGAACTAACGGATTT |
| | AAGCGCGGCAGTGTCGCGTGACTATAACCTGC |
| | AAAGCCTCCCAGGACGTTGGAACTGCCGTTGC |
| | TTGGTACCAGCAGAAACCCGGCAAGGCACCTA |
| | AGCTGCTGATCTCACTGCCGGACGAAGCAACA |
| | TATGTTGGTCGCCCTTATTACTACCA |
| | (SEQ ID NO: 754) |
| tigatuzumab-BtsI-20-8 | CCCTTTAATCAGATGCGTCGAACTAACGGATTT |
| | AAGCGCGGCAGTGAAGGCACCTAAGCTGCTGA |
| | TCTACTGGGCTAGCACAAGGCATACTGGGGTG |
| | CCCAGCCGCTTCTCCGGTTCCGGCAGCGGTAC |
| | AGATTTCACACCACTGCCGGACGAAGCAACAT |
| | ATGTTGGTCGCCCTTATTACTACCA |
| | (SEQ ID NO: 755) |
| tigatuzumab-BtsI-20-9 | CCCTTTAATCAGATGCGTCGAACTAACGGATTT |
| | AAGCGCGGCAGTGCGGCAGCGGTACAGATTTC |
| | ACACTCACTATTAGCTCTCTGCAGCCTGAAGAC |
| | TTCGCCACCTACTATTGCCAGCAGTACTCTAGC |
| | TACCGGACCTCACTGCCGGACGAAGCAACATA |
| | TGTTGGTCGCCCTTATTACTACCA |
| | (SEQ ID NO: 756) |
| tigatuzumab-BtsI-20-10 | CCCTTTAATCAGATGCGTCGAACTAACGGATTT |
| | AAGCGCGGCAGTGAGCAGTACTCTAGCTACCG |
| | GACCTTCGGACAGGGAACAAAAGTGGAGATCA |
| | AGGGGCCCGTAGGCTGAACGACCTATCATTCA |
| | CTGCCGGACGAAGCAACATATGTTGGTCGCCC |
| | TTATTACTACCA (SEQ ID NO: 757) |
| Racotumomab-BtsI-20-0 | CCCTTTAATCAGATGCGTCGCATTTTCTGTTCC |
| | CCAGTGGGCAGTGTTCTTTTATGTTCCTCGCA |
| | GGGGGCCCAGCCGGCCAGGCGCCAGGTGCAG |
| | CTGCAGCAGTCCGGCGCCGAGCTGGTGAAGC |
| | CAGGTGCATCTGTTCACTGCGGGGTGACAATC |
| | TAACTCGAGGTCGCCCTTATTACTACCA |
| | (SEQ ID NO: 758) |
| Racotumomab-BtsI-20-1 | CCCTTTAATCAGATGCGTCGCATTTTCTGTTCC |
| | CCAGTGGGCAGTGGGTGAAGCCAGGTGCATC |
| | TGTTAAGCTGTCCTGCAAGGCATCCGGCTATA |
| | CTTTCACCTCCTACGATATCAACTGGGTTCGGC |
| | AGAGGCCCACTGCGGGGTGACAATCTAACTCG |
| | AGGTCGCCCTTATTACTACCA |
| | (SEQ ID NO: 759) |
| Racotumomab-BtsI-20-2 | CCCTTTAATCAGATGCGTCGCATTTTCTGTTCC |
| | CCAGTGGGCAGTGACTGGGTTCGGCAGAGGC |
| | CTGAGCAAGGACTGGAGTGGATTGGGTGGAT |
| | CTTCCCCGGAGATGGATCTACCAAGTATAACG |
| | AGAAGTTCAAGGGGAACACTGCGGGGTGACA |
| | ATCTAACTCGAGGTCGCCCTTATTACTACCA |
| | (SEQ ID NO: 760) |
| Racotumomab-BtsI-20-3 | CCCTTTAATCAGATGCGTCGCATTTTCTGTTCC |
| | CCAGTGGGCAGTGAAGTATAACGAGAAGTTCA |
| | AGGGGAAAGCCACCCTGACCACAGATAAAAGC |
| | TCAAGCACCGCCTATATGCAGCTCTCTCGGCT |
| | GACATCTGAAGACACTGCGGGGTGACAATCTA |
| | ACTCGAGGTCGCCCTTATTACTACCA |
| | (SEQ ID NO: 761) |
| Racotumomab-BtsI-20-4 | CCCTTTAATCAGATGCGTCGCATTTTCTGTTCC |
| | CCAGTGGGCAGTGGCTCTCTCGGCTGACATCT |
| | GAAGATTCTGCCGTCTATTTTTGCGCTCGGGAG |
| | GACTACTACGACAACTCATATTATTTTGACTAC |
| | TGGGGTCAGGGCACTGCGGGGTGACAATCTAA |
| | CTCGAGGTCGCCCTTATTACTACCA |
| | (SEQ ID NO: 762) |
| Racotumomab-BtsI-20-5 | CCCTTTAATCAGATGCGTCGCATTTTCTGTTCC |
| | CCAGTGGGCAGTGATTATTTTGACTACTGGGG |
| | TCAGGGGACAACACTCACTGTCTCCAGCGGCG |
| | GCTCAGGTGGGAGCGGCGGGGCTTCTGGTGCC |
| | GGATCCGGCACTGCGGGGTGACAATCTAACTC |
| | GAGGTCGCCCTTATTACTACCA |
| | (SEQ ID NO: 763) |
| Racotumomab-BtsI-20-6 | CCCTTTAATCAGATGCGTCGCATTTTCTGTTCC |
| | CCAGTGGGCAGTGGCTTCTGGTGCCGGATCCG |
| | GAGGCGGTGATATCCAGATGACCCAGACAACT |
| | TCAAGCCTGTCCGCCTCACTGGGGGATCGGGT |
| | CACCATTTCTTGCACTGCGGGGTGACAATCTA |
| | ACTCGAGGTCGCCCTTATTACTACCA |
| | (SEQ ID NO: 764) |
| Racotumomab-BtsI-20-7 | CCCTTTAATCAGATGCGTCGCATTTTCTGTTCC |
| | CCAGTGGGCAGTGGGGGATCGGGTCACCATT |
| | TCTTGCAGAGCCTCTCAGGATATCAGCAATTAC |
| | CTGAATTGGTACCAGCAAAAACCCGATGGAAC |
| | AGTGAAACTGCTCACTGCGGGGTGACAATCTA |
| | ACTCGAGGTCGCCCTTATTACTACCA |
| | (SEQ ID NO: 765) |
| Racotumomab-BtsI-20-8 | CCCTTTAATCAGATGCGTCGCATTTTCTGTTCC |
| | CCAGTGGGCAGTGACCCGATGGAACAGTGAA |
| | ACTGCTGATCTACTACACATCTCGGCTGCATA |
| | GCGGAGTGCCCTCCAGGTTCAGCGGCTCCGG |
| | GTCTGGCACAGACTCACTGCGGGGTGACAAT |
| | CTAACTCGAGGTCGCCCTTATTACTACCA |
| | (SEQ ID NO: 766) |
| Racotumomab-BtsI-20-9 | CCCTTTAATCAGATGCGTCGCATTTTCTGTTCC |
| | CCAGTGGGCAGTGTCCGGGTCTGGCACAGAC |
| | TACAGCCTGACCATCAGCAACCTGGAACAGGA |
| | GGACATTGCCACCTATTTTTGTCAACAAGGAAA |
| | TACCCTCCCTTGCACTGCGGGGTGACAATCTA |
| | ACTCGAGGTCGCCCTTATTACTACCA |
| | (SEQ ID NO: 767) |
| Racotumomab-BtsI-20-10 | CCCTTTAATCAGATGCGTCGCATTTTCTGTTCC |
| | CCAGTGGGCAGTGTCAACAAGGAAATACCCTC |
| | CCTTGGACATTTGGGGGAGGCACCAAGCTGGA |
| | AATTAAGGGGCCCAGTGCTTATGAAAGTCCCG |
| | ATTCACTGCGGGGTGACAATCTAACTCGAGGT |
| | CGCCCTTATTACTACCA (SEQ ID NO: 768) |
| conatumumab-BtsI-20-0 | CCCTTTAATCAGATGCGTCGATTTGCCTAACCA |
| | CTCCACTGCAGTGTTGTGGGCGTTAGCAAATT |
| | ACAGGCCCAGCCGGCCAGGCGCCAGGTGCAA |
| | CTCCAGGAATCCGGTCCCGGCCTGGTGAAGCC |
| | ATCTCAGACACTGTCACTGCACTGTACCGAAAA |
| | GCTCTGAGGTCGCCCTTATTACTACCA |
| | (SEQ ID NO: 769) |
| conatumumab-BtsI-20-1 | CCCTTTAATCAGATGCGTCGATTTGCCTAACCA |
| | CTCCACTGCAGTGTGGTGAAGCCATCTCAGAC |
| | ACTGTCCCTGACCTGCACAGTTTCCGGCGGCA |
| | GCATCTCTAGCGGAGACTATTTCTGGTCCTGG |
| | ATCAGACAGCTCCCACTGCACTGTACCGAAAA |
| | GCTCTGAGGTCGCCCTTATTACTACCA |
| | (SEQ ID NO: 770) |
| conatumumab-BtsI-20-2 | CCCTTTAATCAGATGCGTCGATTTGCCTAACCA |
| | CTCCACTGCAGTGTGGTCCTGGATCAGACAGC |
| | TCCCAGGCAAGGGCCTGGAGTGGATAGGGCA |
| | TATTCATAACTCTGGAACAACCTACTATAATCC |
| | CTCTCTCAAATCACGGGCACTGCACTGTACCGA |
| | AAAGCTCTGAGGTCGCCCTTATTACTACCA |
| | (SEQ ID NO: 771) |
| conatumumab-BtsI-20-3 | CCCTTTAATCAGATGCGTCGATTTGCCTAACCAC |
| | TCCACTGCAGTGTACTATAATCCCTCTCTCAAAT |
| | CACGGGTTACTATCTCCGTGGACACTTCCAAGA |
| | AACAGTTCTCCCTCAGACTGTCCTCAGTTACCGC |
| | AGCCGCACTGCACTGTACCGAAAAGCTCTGAGG |
| | TCGCCCTTATTACTACCA (SEQ ID NO: 772) |
| conatumumab-BtsI-20-4 | CCCTTTAATCAGATGCGTCGATTTGCCTAACCA |
| | CTCCACTGCAGTGCTGTCCTCAGTTACCGCAGC |
| | CGACACCGCTGTGTATTACTGCGCAAGGGACAG |
| | GGGGGGCGACTATTACTACGGCATGGACGTGTG |
| | GGGCCAAGGTCACTGCACTGTACCGAAAAGCTC |
| | TGAGGTCGCCCTTATTACTACCA |
| | (SEQ ID NO: 773) |
| conatumumab-BtsI-20-5 | CCCTTTAATCAGATGCGTCGATTTGCCTAACCAC |
| | TCCACTGCAGTGTGGACGTGTGGGGCCAAGGTA |
| | CAACTGTTACCGTTTCCTCAGGTGGATCAGCCG |
| | GCAGCGGATCTTCTGGTGGCGCCTCCGGATCTG |
| | GCGGAGAAACACTGCACTGTACCGAAAAGCTCT |
| | GAGGTCGCCCTTATTACTACCA |
| | (SEQ ID NO: 774) |
| conatumumab-BtsI-20-6 | CCCTTTAATCAGATGCGTCGATTTGCCTAACCAC |
| | TCCACTGCAGTGCTCCGGATCTGGCGGAGAAAT |
| | TGTGCTCACTCAATCCCCAGGGACACTGTCCCT |
| | CAGCCCTGGCGAACGGGCCACTCTGTCCTGCAG |
| | GGCTAGCCACTGCACTGTACCGAAAAGCTCTGA |
| | GGTCGCCCTTATTACTACCA |
| | (SEQ ID NO: 775) |
| conatumumab-BtsI-20-7 | CCCTTTAATCAGATGCGTCGATTTGCCTAACCAC |
| | TCCACTGCAGTGCACTCTGTCCTGCAGGGCTAG |
| | CCAGGGCATTAGCCGGAGCTACCTGGCCTGGTA |
| | TCAGCAAAAGCCTGGGCAGGCCCCCTCTCTGCT |
| | GATCTATGGCACTGCACTGTACCGAAAAGCTCT |
| | GAGGTCGCCCTTATTACTACCA |
| | (SEQ ID NO: 776) |
| conatumumab-BtsI-20-8 | CCCTTTAATCAGATGCGTCGATTTGCCTAACCAC |
| | TCCACTGCAGTGGGCCCCCTCTCTGCTGATCTA |
| | TGGTGCATCCTCCCGCGCCACCGGGATCCCTGA |
| | CAGATTTTCCGGATCCGGTAGCGGTACAGACTTC |
| | ACTCTGACCACTGCACTGTACCGAAAAGCTCTGA |
| | GGTCGCCCTTATTACTACCA |
| | (SEQ ID NO: 777) |
| conatumumab-BtsI-20-9 | CCCTTTAATCAGATGCGTCGATTTGCCTAACCAC |
| | TCCACTGCAGTGTAGCGGTACAGACTTCACTCT |
| | GACAATTTCCCGCCTGGAGCCCGAGGATTTTGC |
| | TGTGTATTACTGCCAGCAATTTGGTTCTTCACCA |
| | TGGACCTTCACTGCACTGTACCGAAAAGCTCTG |
| | AGGTCGCCCTTATTACTACCA |
| | (SEQ ID NO: 778) |
| conatumumab-BtsI-20-10 | CCCTTTAATCAGATGCGTCGATTTGCCTAACCAC |
| | TCCACTGCAGTGATTTGGTTCTTCACCATGGACC |
| | TTTGGTCAAGGGACAAAGGTGGAAATAAAGGGG |
| | CCCCCGAACTGGACGCATAAAATTTCACTGCACT |
| | GTACCGAAAAGCTCTGAGGTCGCCCTTATTACTA |
| | CCA (SEQ ID NO: 779) |
| afutuzumab-BtsI-20-0 | CCCTTTAATCAGATGCGTCGTGACTTATGAACCT |
| | TTGCGCGCAGTGTTAGAGATTATTAGGCGTGGG |
| | GGGCCCAGCCGGCCAGGCGCCAGGTCCAGCTG |
| | GTTCAAAGCGGAGCCGAGGTTAAAAAACCTGGT |
| | TCTAGCGTGAACACTGCATTAACGACTACTCCTG |
| | GGCGGTCGCCCTTATTACTACCA |
| | (SEQ ID NO: 780) |
| afutuzumab-BtsI-20-1 | CCCTTTAATCAGATGCGTCGTGACTTATGAACCT |
| | TTGCGCGCAGTGTAAAAAACCTGGTTCTAGCGT |
| | GAAAGTGAGCTGCAAGGCCTCTGGCTACGCATT |
| | CTCTTACAGCTGGATCAATTGGGTGCGCCAGGC |
| | CCCAGGTCAGCACTGCATTAACGACTACTCCTG |
| | GGCGGTCGCCCTTATTACTACCA |
| | (SEQ ID NO: 781) |
| afutuzumab-BtsI-20-2 | CCCTTTAATCAGATGCGTCGTGACTTATGAACCT |
| | TTGCGCGCAGTGCGCCAGGCCCCAGGTCAGGGT |
| | CTGGAGTGGATGGGCAGGATCTTTCCAGGAGAC |
| | GGAGATACCGATTACAACGGCAAGTTTAAAGGG |
| | AGGGTGACTACACTGCATTAACGACTACTCCTGG |
| | GCGGTCGCCCTTATTACTACCA |
| | (SEQ ID NO: 782) |
| afutuzumab-BtsI-20-3 | CCCTTTAATCAGATGCGTCGTGACTTATGAACCT |
| | TTGCGCGCAGTGGCAAGTTTAAAGGGAGGGTGA |
| | CTATAACCGCTGACAAGAGCACTTCAACAGCCT |
| | ATATGGAACTCAGCTCTCTCAGAAGCGAGGATAC |
| | AGCAGTCTCACTGCATTAACGACTACTCCTGGGC |
| | GGTCGCCCTTATTACTACCA (SEQ ID NO: 783) |
| afutuzumab-BtsI-20-4 | CCCTTTAATCAGATGCGTCGTGACTTATGAACCT |
| | TTGCGCGCAGTGCAGAAGCGAGGATACAGCAGT |
| | CTACTATTGTGCTCGGAATGTCTTTGACGGGTAC |
| | TGGCTGGTGTACTGGGGCCAGGGAACCCTGGTC |
| | ACAGTTAGCCACTGCATTAACGACTACTCCTGGG |
| | CGGTCGCCCTTATTACTACCA (SEQ ID NO: 784) |
| afutuzumab-BtsI-20-5 | CCCTTTAATCAGATGCGTCGTGACTTATGAACCT |
| | TTGCGCGCAGTGAGGGAACCCTGGTCACAGTTA |
| | GCAGCGCAGGTGGGGCCGGCTCTGGGGCAGGG |
| | AGCGGCTCCTCTGGCGCCGGCAGCGGGGACATA |
| | GTGATGACACACACTGCATTAACGACTACTCCTG |
| | GGCGGTCGCCCTTATTACTACCA |
| | (SEQ ID NO: 785) |
| afutuzumab-BtsI-20-6 | CCCTTTAATCAGATGCGTCGTGACTTATGAACCT |
| | TTGCGCGCAGTGAGCGGGGACATAGTGATGACA |
| | CAAACTCCTCTGTCTCTGCCAGTTACCCCCGGAG |
| | AACCCGCCAGCATTTCTTGTAGATCCTCTAAAAG |
| | CCTGCTGCCACTGCATTAACGACTACTCCTGGGC |
| | GGTCGCCCTTATTACTACCA (SEQ ID NO: 786) |
| afutuzumab-BtsI-20-7 | CCCTTTAATCAGATGCGTCGTGACTTATGAACCT |
| | TTGCGCGCAGTGTGTAGATCCTCTAAAAGCCTG |
| | CTGCATAGCAATGGGATCACCTACCTGTACTGG |
| | TATCTGCAGAAACCCGGCCAATCCCCTCAGCTG |
| | CTGATTTACACTGCATTAACGACTACTCCTGGGC |
| | GGTCGCCCTTATTACTACCA (SEQ ID NO: 787) |
| afutuzumab-BtsI-20-8 | CCCTTTAATCAGATGCGTCGTGACTTATGAACCT |
| | TTGCGCGCAGTGAATCCCCTCAGCTGCTGATTT |
| | ACCAAATGTCCAACCTGGTGTCAGGAGTCCCAG |
| | ATCGGTTCAGCGGATCCGGAAGCGGTACTGATT |
| | TTACCCTCAACACTGCATTAACGACTACTCCTGG |
| | GCGGTCGCCCTTATTACTACCA |
| | (SEQ ID NO: 788) |
| afutuzumab-BtsI-20-9 | CCCTTTAATCAGATGCGTCGTGACTTATGAACCT |
| | TTGCGCGCAGTGGAAGCGGTACTGATTTTACCC |
| | TCAAAATATCAAGGGTGGAAGCCGAGGACGTGG |
| | GCGTGTACTATTGCGCCCAGAATCTGGAACTCCC |
| | TTATACATTCACTGCATTAACGACTACTCCTGGG |
| | CGGTCGCCCTTATTACTACCA (SEQ ID NO: 789) |
| afutuzumab-BtsI-20-10 | CCCTTTAATCAGATGCGTCGTGACTTATGAACCT |
| | TTGCGCGCAGTGCAGAATCTGGAACTCCCTTATA |
| | CATTCGGAGGCGGCACAAAAGTGGAAATAAAAG |
| | GGCCCTGAAGGGAAATACCAGCCTTTTCACTGCA |
| | TTAACGACTACTCCTGGGCGGTCGCCCTTATTAC |
| | TACCA (SEQ ID NO: 790) |
| oportuzumab-BtsI-20-0 | CCCTTTAATCAGATGCGTCGATAGGATTAGCTGAT |
| | GGGCCGCAGTGTTTAGGATTACTGCTCGGTGACG |
| | GCCCAGCCGGCCAGGCGCGAGGTGCAGCTGGTG |
| | CAAAGCGGGCCAGGCCTCGTCCAGCCTGGGGGAT |
| | CTGTTACACTGCGACCTTAGTCGGAACACAGAGGT |
| | CGCCCTTATTACTACCA (SEQ ID NO: 791) |
| oportuzumab-BtsI-20-1 | CCCTTTAATCAGATGCGTCGATAGGATTAGCTGAT |
| | GGGCCGCAGTGTCCAGCCTGGGGGATCTGTTAGA |
| | ATCTCATGTGCTGCCTCAGGATATACTTTTACAAA |
| | CTATGGAATGAATTGGGTGAAGCAGGCACCTGGG |
| | CACTGCGACCTTAGTCGGAACACAGAGGTCGCCC |
| | TTATTACTACCA (SEQ ID NO: 792) |
| oportuzumab-BtsI-20-2 | CCCTTTAATCAGATGCGTCGATAGGATTAGCTGAT |
| | GGGCCGCAGTGTGGGTGAAGCAGGCACCTGGGA |
| | AGGGCCTGGAGTGGATGGGTTGGATTAACACTTA |
| | TACAGGCGAATCAACATATGCCGACTCCTTTAAGG |
| | GCCCACTGCGACCTTAGTCGGAACACAGAGGTCG |
| | CCCTTATTACTACCA (SEQ ID NO: 793) |
| oportuzumab-BtsI-20-3 | CCCTTTAATCAGATGCGTCGATAGGATTAGCTGAT |
| | GGGCCGCAGTGATATGCCGACTCCTTTAAGGGCC |
| | GGTTCACCTTTTCTCTCGACACTTCCGCCAGCGCC |
| | GCCTACCTGCAAATCAACAGCCTGAGGGCCGACA |
| | CTGCGACCTTAGTCGGAACACAGAGGTCGCCCTT |
| | ATTACTACCA (SEQ ID NO: 794) |
| oportuzumab-BtsI-20-4 | CCCTTTAATCAGATGCGTCGATAGGATTAGCTGAT |
| | GGGCCGCAGTGTCAACAGCCTGAGGGCCGAAGAT |
| | ACTGCCGTGTATTATTGCGCAAGATTTGCTATTAAG |
| | GGGGACTACTGGGGTCAAGGGACCCTGCTGACAG |
| | TGCACTGCGACCTTAGTCGGAACACAGAGGTCGC |
| | CCTTATTACTACCA (SEQ ID NO: 795) |
| oportuzumab-BtsI-20-5 | CCCTTTAATCAGATGCGTCGATAGGATTAGCTGAT |
| | GGGCCGCAGTGCAAGGGACCCTGCTGACAGTGTC |
| | CAGCGGCGGGAGCGGCGGTTCCGGCGGAGCTTC |
| | CGGAGCCGGGTCCGGCGGAGGGGATATTCAGAT |
| | GACCCAGCACTGCGACCTTAGTCGGAACACAGAG |
| | GTCGCCCTTATTACTACCA (SEQ ID NO: 796) |
| oportuzumab-BtsI-20-6 | CCCTTTAATCAGATGCGTCGATAGGATTAGCTGAT |
| | GGGCCGCAGTGCGGAGGGGATATTCAGATGACCC |
| | AGTCACCCAGCAGCCTCTCTGCATCTGTGGGGGAC |
| | AGGGTGACCATCACCTGTAGATCAACAAAATCTCT |
| | GCCACTGCGACCTTAGTCGGAACACAGAGGTCGC |
| | CCTTATTACTACCA (SEQ ID NO: 797) |
| oportuzumab-BtsI-20-7 | CCCTTTAATCAGATGCGTCGATAGGATTAGCTGAT |
| | GGGCCGCAGTGTCACCTGTAGATCAACAAAATCTC |
| | TGCTGCATAGCAACGGAATCACTTACCTGTACTGG |
| | TATCAGCAGAAGCCTGGCAAAGCCCCAAAACTGC |
| | CACTGCGACCTTAGTCGGAACACAGAGGTCGCCC |
| | TTATTACTACCA (SEQ ID NO: 798) |
| oportuzumab-BtsI-20-8 | CCCTTTAATCAGATGCGTCGATAGGATTAGCTGAT |
| | GGGCCGCAGTGCCTGGCAAAGCCCCAAAACTGCT |
| | GATCTATCAGATGTCCAATCTCGCATCTGGCGTCC |
| | CATCTAGGTTTAGCTCCTCCGGCTCCGGTACAGAC |
| | TTCACTGCGACCTTAGTCGGAACACAGAGGTCGCC |
| | CTTATTACTACCA (SEQ ID NO: 799) |
| oportuzumab-BtsI-20-9 | CCCTTTAATCAGATGCGTCGATAGGATTAGCTGAT |
| | GGGCCGCAGTGTCCGGCTCCGGTACAGACTTCAC |
| | CCTGACCATATCAAGCCTGCAGCCAGAGGACTTTG |
| | CCACTTACTATTGCGCTCAGAATCTCGAAATCCCTA |
| | GCACTGCGACCTTAGTCGGAACACAGAGGTCGCC |
| | CTTATTACTACCA (SEQ ID NO: 800) |
| oportuzumab-BtsI-20-10 | CCCTTTAATCAGATGCGTCGATAGGATTAGCTGATG |
| | GGCCGCAGTGGCGCTCAGAATCTCGAAATCCCTAG |
| | GACATTTGGACAGGGCACAAAGGTCGAACTGAAAG |
| | GGCCCGCCTAGCAACCAACAGTATGTTCACTGCGA |
| | CCTTAGTCGGAACACAGAGGTCGCCCTTATTACTAC |
| | CA (SEQ ID NO: 801) |
| citatuzumab-BtsI-20-0 | CCCTTTAATCAGATGCGTCGTGAGATTCGGGACTAT |
| | TCGGGCAGTGTTTCGCGTGAGTGGTTCATATAGGCC |
| | CAGCCGGCCAGGCGCGAGGTTCAACTCGTCCAATC |
| | TGGCCCTGGGCTCGTCCAGCCCGGGGGATCCGTCA |
| | CTGCGGTCGGAGTCTAACAACAGAGGTCGCCCTTAT |
| | TACTACCA (SEQ ID NO: 802) |
| citatuzumab-BtsI-20-1 | CCCTTTAATCAGATGCGTCGTGAGATTCGGGACTAT |
| | TCGGGCAGTGCCAGCCCGGGGGATCCGTCCGCATC |
| | TCCTGCGCCGCCTCTGGCTATACCTTCACTAATTAT |
| | GGCATGAACTGGGTTAAACAGGCCCCAGGCACACT |
| | GCGGTCGGAGTCTAACAACAGAGGTCGCCCTTATT |
| | ACTACCA (SEQ ID NO: 803) |
| citatuzumab-BtsI-20-2 | CCCTTTAATCAGATGCGTCGTGAGATTCGGGACTAT |
| | TCGGGCAGTGGGGTTAAACAGGCCCCAGGCAAAGG |
| | TCTGGAGTGGATGGGCTGGATTAATACCTATACCGG |
| | CGAGTCCACATACGCCGATAGCTTTAAGGGGAGGCA |
| | CTGCGGTCGGAGTCTAACAACAGAGGTCGCCCTTAT |
| | TACTACCA (SEQ ID NO: 804) |
| citatuzumab-BtsI-20-3 | CCCTTTAATCAGATGCGTCGTGAGATTCGGGACTAT |
| | TCGGGCAGTGACGCCGATAGCTTTAAGGGGAGGTT |
| | CACTTTCAGCCTCGATACCAGCGCTTCAGCAGCATA |
| | CCTGCAGATTAACTCTCTGCGCGCCGAAGATACCCA |
| | CTGCGGTCGGAGTCTAACAACAGAGGTCGCCCTTAT |
| | TACTACCA (SEQ ID NO: 805) |
| citatuzumab-BtsI-20-4 | CCCTTTAATCAGATGCGTCGTGAGATTCGGGACTAT |
| | TCGGGCAGTGCTCTGCGCGCCGAAGATACCGCTGT |
| | CTACTATTGCGCCCGGTTCGCTATTAAGGGGGATTA |
| | CTGGGGGCAGGGCACACTCCTGACCGTTTCAAGCC |
| | ACTGCGGTCGGAGTCTAACAACAGAGGTCGCCCTT |
| | ATTACTACCA (SEQ ID NO: 806) |
| citatuzumab-BtsI-20-5 | CCCTTTAATCAGATGCGTCGTGAGATTCGGGACTAT |
| | TCGGGCAGTGGGCACACTCCTGACCGTTTCAAGCG |
| | GCGGGTCCGCCGGCTCCGGCTCATCTGGCGGGGCA |
| | TCTGGGAGCGGAGGGGACATACAAATGACACAGTC |
| | CACTGCGGTCGGAGTCTAACAACAGAGGTCGCCCT |
| | TATTACTACCA (SEQ ID NO: 807) |
| citatuzumab-BtsI-20-6 | CCCTTTAATCAGATGCGTCGTGAGATTCGGGACTAT |
| | TCGGGCAGTGGAGGGGACATACAAATGACACAGTC |
| | TCCAAGCTCTCTGAGCGCTTCTGTGGGGGATCGCGT |
| | CACCATTACATGCAGATCCACAAAATCCCTGCTGCA |
| | CTGCGGTCGGAGTCTAACAACAGAGGTCGCCCTTAT |
| | TACTACCA (SEQ ID NO: 808) |
| citatuzumab-BtsI-20-7 | CCCTTTAATCAGATGCGTCGTGAGATTCGGGACTAT |
| | TCGGGCAGTGTGCAGATCCACAAAATCCCTGCTGCA |
| | TAGCAATGGCATTACTTATCTGTATTGGTACCAGCA |
| | GAAACCTGGCAAAGCTCCCAAACTGCTGATATACAC |
| | TGCGGTCGGAGTCTAACAACAGAGGTCGCCCTTATT |
| | ACTACCA (SEQ ID NO: 809) |
| citatuzumab-BtsI-20-8 | CCCTTTAATCAGATGCGTCGTGAGATTCGGGACTAT |
| | TCGGGCAGTGCAAAGCTCCCAAACTGCTGATATAC |
| | CAGATGTCCAATCTGGCCTCCGGTGTTCCCAGCAG |
| | ATTCTCAAGCTCCGGCAGCGGGACAGACTTTACTC |
| | CACTGCGGTCGGAGTCTAACAACAGAGGTCGCCCT |
| | TATTACTACCA (SEQ ID NO: 810) |
| citatuzumab-BtsI-20-9 | CCCTTTAATCAGATGCGTCGTGAGATTCGGGACTAT |
| | TCGGGCAGTGGGCAGCGGGACAGACTTTACTCTGA |
| | CCATCAGCAGCCTGCAGCCCGAGGATTTCGCCACTT |
| | ACTACTGCGCTCAGAACCTGGAAATCCCAAGAACCA |
| | CTGCGGTCGGAGTCTAACAACAGAGGTCGCCCTTAT |
| | TACTACCA (SEQ ID NO: 811) |
| citatuzumab-BtsI-20-10 | CCCTTTAATCAGATGCGTCGTGAGATTCGGGACTAT |
| | TCGGGCAGTGTCAGAACCTGGAAATCCCAAGAACA |
| | TTTGGCCAGGGCACTAAGGTTGAACTGAAGGGGCC |
| | CAACGGCGGAATCCAGTATATTTCACTGCGGTCGGA |
| | GTCTAACAACAGAGGTCGCCCTTATTACTACCA |
| | (SEQ ID NO: 812) |
| siltuximab-BtsI-20-0 | CCCTTTAATCAGATGCGTCGTTGGTTAGTACACGG |
| | GACTCGCAGTGTTCAATAGATACCCACCCGTCAGG |
| | CCCAGCCGGCCAGGCGCGAGGTGCAGCTGGTTGA |
| | GTCTGGTGGGAAACTGCTCAAGCCCGGAGGCTCA |
| | CTGCACTGCAGTCCCAAGTTCAGACGTACGGTCGC |
| | CCTTATTACTACCA (SEQ ID NO: 813) |
| siltuximab-BtsI-20-1 | CCCTTTAATCAGATGCGTCGTTGGTTAGTACACGGG |
| | ACTCGCAGTGCAAGCCCGGAGGCTCACTGAAGCTG |
| | TCTTGTGCTGCTTCTGGCTTTACCTTCAGCAGCTTCG |
| | CAATGTCTTGGTTTCGGCAAAGCCCAGAGAACACTG |
| | CAGTCCCAAGTTCAGACGTACGGTCGCCCTTATTAC |
| | TACCA (SEQ ID NO: 814) |
| siltuximab-BtsI-20-2 | CCCTTTAATCAGATGCGTCGTTGGTTAGTACACG |
| | GGACTCGCAGTGGGTTTCGGCAAAGCCCAGAGA |
| | AGCGCCTGGAGTGGGTTGCCGAGATATCTTCTGG |
| | AGGGTCATACACCTACTACCCCGACACTGTTACA |
| | GGTCGGCACTGCAGTCCCAAGTTCAGACGTACG |
| | GTCGCCCTTATTACTACCA (SEQ ID NO: 815) |
| siltuximab-BtsI-20-3 | CCCTTTAATCAGATGCGTCGTTGGTTAGTACACGG |
| | GACTCGCAGTGACCCCGACACTGTTACAGGTCGG |
| | TTCACCATCTCCAGGGATAATGCCAAGAATACCCT |
| | GTATCTGGAGATGTCTTCTCTCAGGTCAGAAGATA |
| | CCGCCACTGCAGTCCCAAGTTCAGACGTACGGTC |
| | GCCCTTATTACTACCA (SEQ ID NO: 816) |
| siltuximab-BtsI-20-4 | CCCTTTAATCAGATGCGTCGTTGGTTAGTACACGG |
| | GACTCGCAGTGTCTTCTCTCAGGTCAGAAGATACC |
| | GCTATGTACTATTGCGCTAGAGGTCTCTGGGGTTA |
| | TTATGCACTCGATTACTGGGGCCAGGGTACTAGCG |
| | TCACTGCAGTCCCAAGTTCAGACGTACGGTCGCCC |
| | TTATTACTACCA (SEQ ID NO: 817) |
| siltuximab-BtsI-20-5 | CCCTTTAATCAGATGCGTCGTTGGTTAGTACACGG |
| | GACTCGCAGTGTGGGGCCAGGGTACTAGCGTCAC |
| | AGTGTCCTCTGGTGGGGCCGGCTCTGGAGCCGGG |
| | AGCGGGTCAAGCGGAGCCGGATCTGGCCAGATTG |
| | TCCTCACTGCAGTCCCAAGTTCAGACGTACGGTCG |
| | CCCTTATTACTACCA (SEQ ID NO: 818) |
| siltuximab-BtsI-20-6 | CCCTTTAATCAGATGCGTCGTTGGTTAGTACACGG |
| | GACTCGCAGTGGCCGGATCTGGCCAGATTGTCCTC |
| | ATCCAGTCCCCCGCCATCATGTCTGCTTCTCCAGG |
| | AGAGAAGGTCACCATGACATGTTCCGCATCATCCT |
| | CCACTGCAGTCCCAAGTTCAGACGTACGGTCGCCC |
| | TTATTACTACCA (SEQ ID NO: 819) |
| siltuximab-BtsI-20-7 | CCCTTTAATCAGATGCGTCGTTGGTTAGTACACGG |
| | GACTCGCAGTGCATGACATGTTCCGCATCATCCTC |
| | CGTTTCTTACATGTATTGGTATCAGCAGAAGCCAG |
| | GCTCTAGCCCACGCCTGCTGATCTATGACACTTCT |
| | ACACTGCAGTCCCAAGTTCAGACGTACGGTCGCCC |
| | TTATTACTACCA (SEQ ID NO: 820) |
| siltuximab-BtsI-20-8 | CCCTTTAATCAGATGCGTCGTTGGTTAGTACACGG |
| | GACTCGCAGTGCGCCTGCTGATCTATGACACTTCT |
| | AACCTCGCCTCCGGAGTGCCCGTGCGCTTTTCCGG |
| | CTCAGGCAGCGGAACATCATATAGCCTGACCATAA |
| | GCCGCACTGCAGTCCCAAGTTCAGACGTACGGTC |
| | GCCCTTATTACTACCA (SEQ ID NO: 821) |
| siltuximab-BtsI-20-9 | CCCTTTAATCAGATGCGTCGTTGGTTAGTACACGG |
| | GACTCGCAGTGAACATCATATAGCCTGACCATAAG |
| | CCGCATGGAAGCCGAGGATGCCGCAACCTATTAT |
| | TGTCAACAGTGGTCAGGGTATCCCTACACATTCGG |
| | GGCACTGCAGTCCCAAGTTCAGACGTACGGTCGC |
| | CCTTATTACTACCA (SEQ ID NO: 822) |
| siltuximab-BtsI-20-10 | CCCTTTAATCAGATGCGTCGTTGGTTAGTACACGG |
| | GACTCGCAGTGCAGGGTATCCCTACACATTCGGG |
| | GGAGGCACCAAACTGGAAATTAAGGGGCCCAGTG |
| | CCAAGGGTTCATAAGTTTCACTGCAGTCCCAAGTT |
| | CAGACGTACGGTCGCCCTTATTACTACCA |
| | (SEQ ID NO: 823) |
| rafivirumab-BtsI-20-0 | CCCTTTAATCAGATGCGTCGATTTGTGTATCGAG |
| | GCTCGTGCAGTGTTATATATCCGCCGTTGTACGT |
| | GGCCCAGCCGGCCAGGCGCCAAGTGCAGCTGGT |
| | TCAGTCCGGGGCCGAAGTCAAGAAGCCTGGGTC |
| | TAGCGTGCACTGCGGTTAAACAATCGCGTGTCTG |
| | GTCGCCCTTATTACTACCA (SEQ ID NO: 824) |
| rafivirumab-BtsI-20-1 | CCCTTTAATCAGATGCGTCGATTTGTGTATCGAG |
| | GCTCGTGCAGTGAAGAAGCCTGGGTCTAGCGTG |
| | AAGGTCTCTTGCAAAGCCAGCGGGGGAACTTTC |
| | AACCGGTATACTGTTAACTGGGTGCGGCAAGCT |
| | CCTGGCCAGGGCACTGCGGTTAAACAATCGCG |
| | TGTCTGGTCGCCCTTATTACTACCA |
| | (SEQ ID NO: 825) |
| rafivirumab-BtsI-20-2 | CCCTTTAATCAGATGCGTCGATTTGTGTATCGAG |
| | GCTCGTGCAGTGCGGCAAGCTCCTGGCCAGGGA |
| | CTGGAGTGGATGGGGGGAATCATCCCCATATTT |
| | GGAACCGCTAACTATGCACAGCGCTTCCAGGGC |
| | AGACTGACTATCACTGCGGTTAAACAATCGCGTG |
| | TCTGGTCGCCCTTATTACTACCA |
| | (SEQ ID NO: 826) |
| rafivirumab-BtsI-20-3 | CCCTTTAATCAGATGCGTCGATTTGTGTATCGAG |
| | GCTCGTGCAGTGGCTTCCAGGGCAGACTGACTA |
| | TAACCGCAGATGAGTCCACCTCAACCGCCTACAT |
| | GGAGCTGTCCTCTCTGCGGTCCGACGATACAGC |
| | CGTGTACTTTCACTGCGGTTAAACAATCGCGTGT |
| | CTGGTCGCCCTTATTACTACCA |
| | (SEQ ID NO: 827) |
| rafivirumab-BtsI-20-4 | CCCTTTAATCAGATGCGTCGATTTGTGTATCGAG |
| | GCTCGTGCAGTGCCGACGATACAGCCGTGTACT |
| | TTTGCGCCCGGGAGAACCTGGACAACTCTGGCA |
| | CTTACTATTACTTCAGCGGCTGGTTCGACCCTTG |
| | GGGACAAGGCCACTGCGGTTAAACAATCGCGTG |
| | TCTGGTCGCCCTTATTACTACCA |
| | (SEQ ID NO: 828) |
| rafivirumab-BtsI-20-5 | CCCTTTAATCAGATGCGTCGATTTGTGTATCGAG |
| | GCTCGTGCAGTGTTCGACCCTTGGGGACAAGGC |
| | ACCAGCGTCACAGTCTCATCTGGCGGTTCTGGG |
| | GGGAGCGGCGGCGCTTCTGGGGCCGGAAGCGG |
| | TGGCGGTCAGAGCACTGCGGTTAAACAATCGCG |
| | TGTCTGGTCGCCCTTATTACTACCA |
| | (SEQ ID NO: 829) |
| rafivirumab-BtsI-20-6 | CCCTTTAATCAGATGCGTCGATTTGTGTATCGAG |
| | GCTCGTGCAGTGAAGCGGTGGCGGTCAGAGCG |
| | CACTGACCCAGCCTCGCAGCGTCTCCGGCTCCC |
| | CTGGGCAGAGCGTGACAATATCTTGTACAGGCA |
| | CCTCCTCCGACACTGCGGTTAAACAATCGCGTGT |
| | CTGGTCGCCCTTATTACTACCA |
| | (SEQ ID NO: 830) |
| rafivirumab-BtsI-20-7 | CCCTTTAATCAGATGCGTCGATTTGTGTATCGAG |
| | GCTCGTGCAGTGCTTGTACAGGCACCTCCTCCGA |
| | TATCGGGGGGTATAATTTCGTGTCATGGTACCAG |
| | CAACATCCCGGCAAAGCCCCAAAGCTGATGATCT |
| | ACGACGCCCACTGCGGTTAAACAATCGCGTGTCT |
| | GGTCGCCCTTATTACTACCA (SEQ ID NO: 831) |
| rafivirumab-BtsI-20-8 | CCCTTTAATCAGATGCGTCGATTTGTGTATCGAG |
| | GCTCGTGCAGTGCCAAAGCTGATGATCTACGAC |
| | GCCACTAAGAGGCCTTCCGGGGTGCCCGATAGG |
| | TTCAGCGGGAGCAAATCTGGTAATACTGCCTCA |
| | CTGACTATATCAGGCACTGCGGTTAAACAATCGC |
| | GTGTCTGGTCGCCCTTATTACTACCA |
| | (SEQ ID NO: 832) |
| rafivirumab-BtsI-20-9 | CCCTTTAATCAGATGCGTCGATTTGTGTATCGAG |
| | GCTCGTGCAGTGTAATACTGCCTCACTGACTATA |
| | TCAGGCCTGCAGGCAGAAGACGAGGCAGATTAT |
| | TACTGCTGTTCTTACGCCGGTGACTACACACCTG |
| | GTGTGGCACTGCGGTTAAACAATCGCGTGTCTG |
| | GTCGCCCTTATTACTACCA (SEQ ID NO: 833) |
| rafivirumab-BtsI-20-10 | CCCTTTAATCAGATGCGTCGATTTGTGTATCGAG |
| | GCTCGTGCAGTGGGTGACTACACACCTGGTGTG |
| | GTGTTTGGGGGCGGCACCAAGCTGACTGTGCTG |
| | GGGCCCACCGAACGGCATACATCTATTTCACTG |
| | CGGTTAAACAATCGCGTGTCTGGTCGCCCTTATT |
| | ACTACCA (SEQ ID NO: 834) |
| Foravirumab-BtsI-20-0 | CCCTTTAATCAGATGCGTCGATCGTTCCCCATCA |
| | CATTCTGCAGTGTTCGAGAGTCTCCCACGATATC |
| | GGCCCAGCCGGCCAGGCGCCAGGTCCAGCTGGT |
| | CGAGTCTGGCGGAGGCGCCGTGCAGCCCGGGAG |
| | GTCCCTCACTGCTAAGTGCTCAAAACGAACGGGG |
| | TCGCCCTTATTACTACCA (SEQ ID NO: 835) |
| Foravirumab-BtsI-20-1 | CCCTTTAATCAGATGCGTCGATCGTTCCCCATCA |
| | CATTCTGCAGTGGCAGCCCGGGAGGTCCCTGAG |
| | ACTGTCTTGCGCTGCTTCAGGTTTCACTTTTTCTT |
| | CCTACGGCATGCACTGGGTCCGCCAAGCTCCTG |
| | GAAAGGCACTGCTAAGTGCTCAAAACGAACGGG |
| | GTCGCCCTTATTACTACCA (SEQ ID NO: 836) |
| Foravirumab-BtsI-20-2 | CCCTTTAATCAGATGCGTCGATCGTTCCCCATCA |
| | CATTCTGCAGTGTCCGCCAAGCTCCTGGAAAGG |
| | GACTGGAATGGGTCGCCGTCATACTGTACGACG |
| | GGAGCGACAAGTTTTATGCCGATTCAGTGAAGG |
| | GTCGGTTTCACTGCTAAGTGCTCAAAACGAACG |
| | GGGTCGCCCTTATTACTACCA (SEQ ID NO: 837) |
| Foravirumab-BtsI-20-3 | CCCTTTAATCAGATGCGTCGATCGTTCCCCATCAC |
| | ATTCTGCAGTGCCGATTCAGTGAAGGGTCGGTTT |
| | ACTATTTCACGCGATAATTCCAAGAACACACTGTA |
| | TCTGCAGATGAATTCCCTGCGGGCTGAAGATACA |
| | GCCCACTGCTAAGTGCTCAAAACGAACGGGGTCG |
| | CCCTTATTACTACCA (SEQ ID NO: 838) |
| Foravirumab-BtsI-20-4 | CCCTTTAATCAGATGCGTCGATCGTTCCCCATCAC |
| | ATTCTGCAGTGCCTGCGGGCTGAAGATACAGCCG |
| | TGTACTACTGTGCAAAAGTGGCCGTGGCAGGGAC |
| | TCACTTTGACTATTGGGGCCAGGGGACTCTGGTG |
| | ACTGCACTGCTAAGTGCTCAAAACGAACGGGGTC |
| | GCCCTTATTACTACCA (SEQ ID NO: 839) |
| Foravirumab-BtsI-20-5 | CCCTTTAATCAGATGCGTCGATCGTTCCCCATCAC |
| | ATTCTGCAGTGGCCAGGGGACTCTGGTGACTGTG |
| | TCCTCTGCAGGCGGTTCCGCCGGCTCTGGCTCCA |
| | GCGGGGGCGCTTCAGGCTCCGGGGGCGATATCC |
| | AAATGCACTGCTAAGTGCTCAAAACGAACGGGGT |
| | CGCCCTTATTACTACCA (SEQ ID NO: 840) |
| Foravirumab-BtsI-20-6 | CCCTTTAATCAGATGCGTCGATCGTTCCCCATCAC |
| | ATTCTGCAGTGTCCGGGGGCGATATCCAAATGAC |
| | CCAAAGCCCATCCTCACTCTCCGCCTCTGTTGGCG |
| | ATAGAGTCACTATTACCTGCAGGGCCTCTCAGGCA |
| | CTGCTAAGTGCTCAAAACGAACGGGGTCGCCCTT |
| | ATTACTACCA (SEQ ID NO: 841) |
| Foravirumab-BtsI-20-7 | CCCTTTAATCAGATGCGTCGATCGTTCCCCATCAC |
| | ATTCTGCAGTGTACCTGCAGGGCCTCTCAGGGGA |
| | TCCGCAATGATCTCGGATGGTACCAGCAGAAACC |
| | CGGAAAAGCTCCAAAACTGCTGATATACGCAGCT |
| | TCTTCACTGCTAAGTGCTCAAAACGAACGGGGTC |
| | GCCCTTATTACTACCA (SEQ ID NO: 842) |
| Foravirumab-BtsI-20-8 | CCCTTTAATCAGATGCGTCGATCGTTCCCCATCAC |
| | ATTCTGCAGTGAACTGCTGATATACGCAGCTTCTT |
| | CTCTGCAGTCCGGGGTCCCCTCCCGGTTCTCCGG |
| | TAGCGGTTCTGGAACCGACTTTACACTGACTATAT |
| | CCTCTCACTGCTAAGTGCTCAAAACGAACGGGGTC |
| | GCCCTTATTACTACCA (SEQ ID NO: 843) |
| Foravirumab-BtsI-20-9 | CCCTTTAATCAGATGCGTCGATCGTTCCCCATCAC |
| | ATTCTGCAGTGACCGACTTTACACTGACTATATCC |
| | TCTCTCCAGCCTGAAGACTTCGCTACATATTACTG |
| | CCAGCAGCTGAACAGCTACCCTCCCACATTCGGC |
| | CACTGCTAAGTGCTCAAAACGAACGGGGTCGCCC |
| | TTATTACTACCA (SEQ ID NO: 844) |
| Foravirumab-BtsI-20-10 | CCCTTTAATCAGATGCGTCGATCGTTCCCCATCAC |
| | ATTCTGCAGTGCAGCTACCCTCCCACATTCGGCG |
| | GCGGTACTAAGGTGGAAATCAAAGGGCCCCAAAG |
| | TGCGGAAAACAGAGATTCACTGCTAAGTGCTCAA |
| | AACGAACGGGGTCGCCCTTATTACTACCA (SEQ ID NO: 845) |
| Farletuzumab-BtsI-20-0 | CCCTTTAATCAGATGCGTCGATTACCATGTTATCG |
| | GGCGAGCAGTGTTATTCAGTTGGTCTTACGGGTG |
| | GCCCAGCCGGCCAGGCGCGAAGTTCAGCTCGTG |
| | GAGTCTGGCGGAGGCGTGGTCCAACCTGGCAGG |
| | TCCCACTGCAATCTTGCGTTCCCTAACCTGGTCGC |
| | CCTTATTACTACCA (SEQ ID NO: 846) |
| Farletuzumab-BtsI-20-1 | CCCTTTAATCAGATGCGTCGATTACCATGTTATCG |
| | GGCGAGCAGTGTGGTCCAACCTGGCAGGTCCCTG |
| | AGGCTGTCTTGTTCTGCCAGCGGATTTACATTTTC |
| | CGGGTACGGACTGTCCTGGGTCAGACAGGCTCCA |
| | GGGACACTGCAATCTTGCGTTCCCTAACCTGGTC |
| | GCCCTTATTACTACCA (SEQ ID NO: 847) |
| Farletuzumab-BtsI-20-2 | CCCTTTAATCAGATGCGTCGATTACCATGTTATCG |
| | GGCGAGCAGTGGGGTCAGACAGGCTCCAGGGAA |
| | AGGCCTCGAATGGGTGGCAATGATCTCTAGCGGA |
| | GGCTCATACACCTATTACGCCGACTCCGTCAAGG |
| | GGCACTGCAATCTTGCGTTCCCTAACCTGGTCGCC |
| | CTTATTACTACCA (SEQ ID NO: 848) |
| Farletuzumab-BtsI-20-3 | CCCTTTAATCAGATGCGTCGATTACCATGTTATCG |
| | GGCGAGCAGTGACGCCGACTCCGTCAAGGGGCG |
| | CTTCGCCATCAGCAGAGATAATGCAAAGAATACT |
| | CTCTTCCTCCAGATGGATTCTCTCCGGCCCGAGG |
| | ACACTGCAATCTTGCGTTCCCTAACCTGGTCGCC |
| | CTTATTACTACCA (SEQ ID NO: 849) |
| Farletuzumab-BtsI-20-4 | CCCTTTAATCAGATGCGTCGATTACCATGTTATCG |
| | GGCGAGCAGTGATTCTCTCCGGCCCGAGGACACC |
| | GGTGTGTACTTCTGTGCTCGCCATGGGGATGACC |
| | CAGCCTGGTTTGCTTACTGGGGCCAGGGAACTCC |
| | TGTGACACTGCAATCTTGCGTTCCCTAACCTGGTC |
| | GCCCTTATTACTACCA (SEQ ID NO: 850) |
| Farletuzumab-BtsI-20-5 | CCCTTTAATCAGATGCGTCGATTACCATGTTATCG |
| | GGCGAGCAGTGGGGCCAGGGAACTCCTGTGACC |
| | GTTTCTAGCGGGGGGGCTGGCAGCGGGGCCGGT |
| | TCAGGTTCTTCCGGCGCCGGCTCCGGGGACATCC |
| | AGCTCACCACTGCAATCTTGCGTTCCCTAACCTG |
| | GTCGCCCTTATTACTACCA (SEQ ID NO: 851) |
| Farletuzumab-BtsI-20-6 | CCCTTTAATCAGATGCGTCGATTACCATGTTATCG |
| | GGCGAGCAGTGTCCGGGGACATCCAGCTCACTC |
| | AGAGCCCATCTTCACTGTCAGCATCCGTCGGAGA |
| | TAGAGTGACTATAACCTGTTCAGTGTCCTCATCAA |
| | TCAGCCACTGCAATCTTGCGTTCCCTAACCTGGTC |
| | GCCCTTATTACTACCA (SEQ ID NO: 852) |
| Farletuzumab-BtsI-20-7 | CCCTTTAATCAGATGCGTCGATTACCATGTTATCG |
| | GGCGAGCAGTGCTGTTCAGTGTCCTCATCAATCA |
| | GCTCCAACAATCTGCACTGGTACCAGCAGAAACC |
| | AGGAAAGGCACCAAAACCCTGGATATACGGCAC |
| | CTCAAACACTGCAATCTTGCGTTCCCTAACCTGG |
| | TCGCCCTTATTACTACCA (SEQ ID NO: 853) |
| Farletuzumab-BtsI-20-8 | CCCTTTAATCAGATGCGTCGATTACCATGTTATC |
| | GGGCGAGCAGTGCCCTGGATATACGGCACCTC |
| | AAATCTGGCTTCCGGTGTGCCTTCCAGATTCTC |
| | AGGGAGCGGATCCGGCACCGACTACACCTTTA |
| | CAATCAGCTCCCACTGCAATCTTGCGTTCCCTAA |
| | CCTGGTCGCCCTTATTACTACCA (SEQ ID NO: 854) |
| Farletuzumab-BtsI-20-9 | CCCTTTAATCAGATGCGTCGATTACCATGTTATC |
| | GGGCGAGCAGTGCGACTACACCTTTACAATCAG |
| | CTCCCTGCAGCCCGAGGACATTGCAACATACTA |
| | CTGTCAACAGTGGAGCTCCTATCCCTATATGTAC |
| | ACCTTCGGACCACTGCAATCTTGCGTTCCCTAAC |
| | CTGGTCGCCCTTATTACTACCA (SEQ ID NO: 855) |
| Farletuzumab-BtsI-20-10 | CCCTTTAATCAGATGCGTCGATTACCATGTTATC |
| | GGGCGAGCAGTGCTATCCCTATATGTACACCTT |
| | CGGACAGGGAACAAAGGTTGAGATTAAAGGGCC |
| | CACCGGGAAAGACGAATAACTTTCACTGCAATC |
| | TTGCGTTCCCTAACCTGGTCGCCCTTATTACTAC |
| | CA (SEQ ID NO: 856) |
| Elotuzumab-BtsI-20-0 | CCCTTTAATCAGATGCGTCGTCGGTGGATATGAC |
| | GTAACCGCAGTGTTGGATTGCAACGTCAGGAAAT |
| | GGCCCAGCCGGCCAGGCGCGAGGTGCAGCTCG |
| | TCGAGTCCGGAGGCGGCCTGGTTCAGCCTGGCG |
| | GGTCACTGCAGATAACGAGCACAGTCTGGGGTC |
| | GCCCTTATTACTACCA (SEQ ID NO: 857) |
| Elotuzumab-BtsI-20-1 | CCCTTTAATCAGATGCGTCGTCGGTGGATATGAC |
| | GTAACCGCAGTGCTGGTTCAGCCTGGCGGGTCT |
| | CTCCGCCTGTCCTGCGCCGCCTCCGGATTCGACT |
| | TTAGCAGATACTGGATGTCCTGGGTGAGACAGGC |
| | TCCTGGCACTGCAGATAACGAGCACAGTCTGGGG |
| | TCGCCCTTATTACTACCA (SEQ ID NO: 858) |
| Elotuzumab-BtsI-20-2 | CCCTTTAATCAGATGCGTCGTCGGTGGATATGAC |
| | GTAACCGCAGTGCTGGGTGAGACAGGCTCCTGG |
| | AAAAGGACTCGAATGGATCGGGGAGATCAACCC |
| | CGATTCTTCCACCATCAACTACGCACCTAGCCTG |
| | AAAGATCACTGCAGATAACGAGCACAGTCTGGGG |
| | TCGCCCTTATTACTACCA (SEQ ID NO: 859) |
| Elotuzumab-BtsI-20-3 | CCCTTTAATCAGATGCGTCGTCGGTGGATATGAC |
| | GTAACCGCAGTGACTACGCACCTAGCCTGAAAG |
| | ATAAATTCATCATTTCCAGAGACAATGCCAAAAA |
| | TTCACTGTACCTCCAAATGAACAGCCTGAGAGCT |
| | GAGGATCACTGCAGATAACGAGCACAGTCTGGG |
| | GTCGCCCTTATTACTACCA (SEQ ID NO: 860) |
| Elotuzumab-BtsI-20-4 | CCCTTTAATCAGATGCGTCGTCGGTGGATATGAC |
| | GTAACCGCAGTGAACAGCCTGAGAGCTGAGGAT |
| | ACTGCTGTCTACTACTGCGCTAGGCCCGATGGGA |
| | ATTACTGGTACTTCGATGTGTGGGGGCAGGGCA |
| | CTCTGGTCACTGCAGATAACGAGCACAGTCTGG |
| | GGTCGCCCTTATTACTACCA (SEQ ID NO: 861) |
| Elotuzumab-BtsI-20-5 | CCCTTTAATCAGATGCGTCGTCGGTGGATATGAC |
| | GTAACCGCAGTGGGGGGCAGGGCACTCTGGTTA |
| | CCGTGTCATCAGGTGGCTCCGGAGGGTCCGGCG |
| | GCGCAAGCGGAGCCGGATCCGGCGGAGGAGACA |
| | TCCAGATGCACTGCAGATAACGAGCACAGTCTGG |
| | GGTCGCCCTTATTACTACCA (SEQ ID NO: 862) |
| Elotuzumab-BtsI-20-6 | CCCTTTAATCAGATGCGTCGTCGGTGGATATGAC |
| | GTAACCGCAGTGCGGCGGAGGAGACATCCAGAT |
| | GACACAGTCTCCATCCAGCCTCAGCGCCTCCGTT |
| | GGCGATCGGGTGACAATCACCTGCAAGGCCTCA |
| | CAGGACGCACTGCAGATAACGAGCACAGTCTGG |
| | GGTCGCCCTTATTACTACCA (SEQ ID NO: 863) |
| Elotuzumab-BtsI-20-7 | CCCTTTAATCAGATGCGTCGTCGGTGGATATGAC |
| | GTAACCGCAGTGCTGCAAGGCCTCACAGGACGT |
| | CGGAATCGCCGTTGCTTGGTATCAACAAAAGCCC |
| | GGGAAGGTCCCCAAGCTGCTGATTTATTGGGCC |
| | TCTACACCACTGCAGATAACGAGCACAGTCTGG |
| | GGTCGCCCTTATTACTACCA (SEQ ID NO: 864) |
| Elotuzumab-BtsI-20-8 | CCCTTTAATCAGATGCGTCGTCGGTGGATATGA |
| | CGTAACCGCAGTGCTGCTGATTTATTGGGCCTC |
| | TACACGGCACACAGGTGTTCCAGATCGCTTCTC |
| | TGGTAGCGGCTCCGGAACCGACTTTACTCTGAC |
| | TATATCTTCCACTGCAGATAACGAGCACAGTCTG |
| | GGGTCGCCCTTATTACTACCA (SEQ ID NO: 865) |
| Elotuzumab-BtsI-20-9 | CCCTTTAATCAGATGCGTCGTCGGTGGATATGAC |
| | GTAACCGCAGTGGAACCGACTTTACTCTGACTAT |
| | ATCTTCTCTGCAGCCCGAGGATGTGGCCACTTAC |
| | TACTGTCAGCAATATAGCTCCTACCCATACACTTT |
| | TGGCCACTGCAGATAACGAGCACAGTCTGGGGTC |
| | GCCCTTATTACTACCA (SEQ ID NO: 866) |
| Elotuzumab-BtsI-20-10 | CCCTTTAATCAGATGCGTCGTCGGTGGATATGAC |
| | GTAACCGCAGTGTAGCTCCTACCCATACACTTTT |
| | GGCCAGGGGACAAAAGTGGAGATCAAAGGGCCC |
| | GCTTCGTGGAGATTCCTGTATTCACTGCAGATAA |
| | CGAGCACAGTCTGGGGTCGCCCTTATTACTACCA |
| | (SEQ ID NO: 867) |
| necitumumab-BtsI-20-0 | CCCTTTAATCAGATGCGTCGGGTCAGATGGTTTA |
| | CATGCGGCAGTGTTGAATGTTGCAGACTGGAAGG |
| | GGCCCAGCCGGCCAGGCGCCAGGTGCAGCTGCA |
| | AGAATCAGGGCCAGGACTCGTCAAACCCTCTCAA |
| | ACACTGCACTGCATCGCGGATAGAGAACAACTGG |
| | TCGCCCTTATTACTACCA (SEQ ID NO: 868) |
| necitumumab-BtsI-20-1 | CCCTTTAATCAGATGCGTCGGGTCAGATGGTTTA |
| | CATGCGGCAGTGCTCGTCAAACCCTCTCAAACAC |
| | TGTCTCTGACTTGTACCGTGTCTGGGGGCTCCAT |
| | CTCATCCGGGGATTACTACTGGTCATGGATCAGG |
| | CAACCCACTGCATCGCGGATAGAGAACAACTGGT |
| | CGCCCTTATTACTACCA (SEQ ID NO: 869) |
| necitumumab-BtsI-20-2 | CCCTTTAATCAGATGCGTCGGGTCAGATGGTTTA |
| | CATGCGGCAGTGTACTGGTCATGGATCAGGCAAC |
| | CACCTGGCAAAGGTCTGGAGTGGATTGGCTATAT |
| | CTACTACTCTGGGTCAACCGATTATAACCCAAGCC |
| | TCAACACTGCATCGCGGATAGAGAACAACTGGTC |
| | GCCCTTATTACTACCA (SEQ ID NO: 870) |
| necitumumab-BtsI-20-3 | CCCTTTAATCAGATGCGTCGGGTCAGATGGTTTA |
| | CATGCGGCAGTGAACCGATTATAACCCAAGCCTC |
| | AAGTCTCGGGTTACAATGAGCGTGGATACTAGCA |
| | AGAATCAATTCTCACTCAAGGTGAACTCTGTTACT |
| | GCCGCACTGCATCGCGGATAGAGAACAACTGGTC |
| | GCCCTTATTACTACCA (SEQ ID NO: 871) |
| necitumumab-BtsI-20-4 | CCCTTTAATCAGATGCGTCGGGTCAGATGGTTT |
| | ACATGCGGCAGTGTCAAGGTGAACTCTGTTACT |
| | GCCGCTGACACCGCCGTGTACTATTGCGCTCGG |
| | GTCTCTATCTTCGGTGTGGGGACCTTTGACTATT |
| | GGGGTCAAGCACTGCATCGCGGATAGAGAACAA |
| | CTGGTCGCCCTTATTACTACCA (SEQ ID NO: 872) |
| necitumumab-BtsI-20-5 | CCCTTTAATCAGATGCGTCGGGTCAGATGGTTT |
| | ACATGCGGCAGTGGGGACCTTTGACTATTGGGG |
| | TCAAGGAACACTGGTCACTGTTTCAAGCGGCGG |
| | CTCTGCAGGGTCAGGCTCATCCGGAGGCGCCT |
| | CCGCACTGCATCGCGGATAGAGAACAACTGGTC |
| | GCCCTTATTACTACCA (SEQ ID NO: 873) |
| necitumumab-BtsI-20-6 | CCCTTTAATCAGATGCGTCGGGTCAGATGGTTT |
| | ACATGCGGCAGTGCATCCGGAGGCGCCTCCGG |
| | CTCTGGCGGCGAAATAGTGATGACTCAGTCACC |
| | AGCTACTCTGTCCCTCTCCCCTGGAGAGAGGGC |
| | TACACTCTCCACTGCATCGCGGATAGAGAACAA |
| | CTGGTCGCCCTTATTACTACCA (SEQ ID NO: 874) |
| necitumumab-BtsI-20-7 | CCCTTTAATCAGATGCGTCGGGTCAGATGGTTT |
| | ACATGCGGCAGTGCCTGGAGAGAGGGCTACAC |
| | TCTCTTGCCGCGCCTCACAGTCTGTGAGCAGCT |
| | ACCTCGCTTGGTACCAGCAGAAACCAGGTCAGG |
| | CCCCCCACTGCATCGCGGATAGAGAACAACTGG |
| | TCGCCCTTATTACTACCA (SEQ ID NO: 875) |
| necitumumab-BtsI-20-8 | CCCTTTAATCAGATGCGTCGGGTCAGATGGTTT |
| | ACATGCGGCAGTGGAAACCAGGTCAGGCCCCC |
| | CGGCTGCTGATCTATGACGCTAGCAATCGGGCT |
| | ACTGGCATCCCCGCCAGATTTTCTGGATCTGGG |
| | TCAGGCACCACTGCATCGCGGATAGAGAACAAC |
| | TGGTCGCCCTTATTACTACCA (SEQ ID NO: 876) |
| necitumumab-BtsI-20-9 | CCCTTTAATCAGATGCGTCGGGTCAGATGGTTT |
| | ACATGCGGCAGTGTTTCTGGATCTGGGTCAGGC |
| | ACCGACTTCACACTGACTATAAGCTCACTGGAG |
| | CCCGAAGACTTCGCCGTGTATTACTGCCATCAG |
| | TATGGAAGCACACTGCATCGCGGATAGAGAAC |
| | AACTGGTCGCCCTTATTACTACCA |
| | (SEQ ID NO: 877) |
| necitumumab-BtsI-20-10 | CCCTTTAATCAGATGCGTCGGGTCAGATGGTTTA |
| | CATGCGGCAGTGTATTACTGCCATCAGTATGGAA |
| | GCACCCCCCTGACCTTTGGGGGTGGTACCAAAGC |
| | CGAGATTAAGGGGCCCATCTAGTAACAAGCCCGA |
| | GGTTCACTGCATCGCGGATAGAGAACAACTGGTC |
| | GCCCTTATTACTACCA (SEQ ID NO: 878) |
| figitumumab-BtsI-20-0 | CCCTTTAATCAGATGCGTCGTCTCGTTCGAAAAT |
| | CATCGCGCAGTGTTGTCCATGAATACAACACCG |
| | GGGCCCAGCCGGCCAGGCGCGAGGTTCAGCTC |
| | CTGGAGTCCGGGGGCGGACTGGTGCAGCCCGG |
| | GGGCTCACTGACACTGCGTCACCGGCGAGATTT |
| | AATCGGTCGCCCTTATTACTACCA (SEQ ID NO: 879) |
| figitumumab-BtsI-20-1 | CCCTTTAATCAGATGCGTCGTCTCGTTCGAAAAT |
| | CATCGCGCAGTGAGCCCGGGGGCTCACTGAGGC |
| | TGAGCTGCACAGCCTCTGGCTTCACATTTAGCTC |
| | CTACGCCATGAATTGGGTGAGACAAGCCCCTGG |
| | AAAGGGGCACTGCGTCACCGGCGAGATTTAATC |
| | GGTCGCCCTTATTACTACCA (SEQ ID NO: 880) |
| figitumumab-BtsI-20-2 | CCCTTTAATCAGATGCGTCGTCTCGTTCGAAAAT |
| | CATCGCGCAGTGGAGACAAGCCCCTGGAAAGGG |
| | GCTGGAGTGGGTGTCTGCTATTTCAGGCTCAGG |
| | GGGGACAACCTTTTATGCCGACAGCGTGAAGGG |
| | CAGGTTCACCCACTGCGTCACCGGCGAGATTTA |
| | ATCGGTCGCCCTTATTACTACCA (SEQ ID NO: 881) |
| figitumumab-BtsI-20-3 | CCCTTTAATCAGATGCGTCGTCTCGTTCGAAAAT |
| | CATCGCGCAGTGAGCGTGAAGGGCAGGTTCACC |
| | ATTTCACGCGATAACTCACGCACTACCCTCTATC |
| | TGCAGATGAATTCCCTGCGGGCAGAAGACACAG |
| | CCGTCTATTACACTGCGTCACCGGCGAGATTTA |
| | ATCGGTCGCCCTTATTACTACCA |
| | (SEQ ID NO: 882) |
| figitumumab-BtsI-20-4 | CCCTTTAATCAGATGCGTCGTCTCGTTCGAAAAT |
| | CATCGCGCAGTGGGCAGAAGACACAGCCGTCT |
| | ATTATTGTGCAAAAGACCTGGGATGGTCTGACT |
| | CATATTATTATTATTATGGGATGGATGTTTGGGG |
| | GCAGGGGCACTGCGTCACCGGCGAGATTTAAT |
| | CGGTCGCCCTTATTACTACCA (SEQ ID NO: 883) |
| figitumumab-BtsI-20-5 | CCCTTTAATCAGATGCGTCGTCTCGTTCGAAAA |
| | TCATCGCGCAGTGATGGATGTTTGGGGGCAGG |
| | GGACCACCGTGACCGTCAGCAGCGGCGGGGC |
| | AGGATCTGGGGCCGGGTCTGGCTCATCAGGGG |
| | CCGGTTCTGGCACTGCGTCACCGGCGAGATTT |
| | AATCGGTCGCCCTTATTACTACCA (SEQ ID NO: 884) |
| figitumumab-BtsI-20-6 | CCCTTTAATCAGATGCGTCGTCTCGTTCGAAAAT |
| | CATCGCGCAGTGCATCAGGGGCCGGTTCTGGGG |
| | ATATACAGATGACCCAGTTCCCATCATCTCTCTC |
| | AGCCTCTGTCGGGGATAGGGTTACCATTACTTGC |
| | AGAGCCAGCACTGCGTCACCGGCGAGATTTAAT |
| | CGGTCGCCCTTATTACTACCA (SEQ ID NO: 885) |
| figitumumab-BtsI-20-7 | CCCTTTAATCAGATGCGTCGTCTCGTTCGAAAAT |
| | CATCGCGCAGTGGGTTACCATTACTTGCAGAGC |
| | CAGCCAGGGAATCAGAAATGATCTGGGCTGGTA |
| | TCAACAGAAACCAGGTAAAGCCCCCAAGAGGCT |
| | CATCTACGCCACTGCGTCACCGGCGAGATTTAA |
| | TCGGTCGCCCTTATTACTACCA (SEQ ID NO: 886) |
| figitumumab-BtsI-20-8 | CCCTTTAATCAGATGCGTCGTCTCGTTCGAAAAT |
| | CATCGCGCAGTGGCCCCCAAGAGGCTCATCTAC |
| | GCCGCATCCCGCCTGCATCGGGGAGTCCCTTCA |
| | CGCTTTTCCGGCTCTGGCTCAGGTACCGAGTTCA |
| | CTCTCACTACACTGCGTCACCGGCGAGATTTAAT |
| | CGGTCGCCCTTATTACTACCA (SEQ ID NO: 887) |
| figitumumab-BtsI-20-9 | CCCTTTAATCAGATGCGTCGTCTCGTTCGAAAAT |
| | CATCGCGCAGTGCAGGTACCGAGTTCACTCTCA |
| | CTATTTCCAGCCTCCAGCCAGAGGATTTTGCAAC |
| | CTACTACTGCCTGCAACATAATTCTTATCCCTGT |
| | TCATTTGGTCACACTGCGTCACCGGCGAGATTT |
| | AATCGGTCGCCCTTATTACTACCA (SEQ ID NO: 888) |
| figitumumab-BtsI-20-10 | CCCTTTAATCAGATGCGTCGTCTCGTTCGAAAAT |
| | CATCGCGCAGTGTAATTCTTATCCCTGTTCATTT |
| | GGTCAGGGCACAAAGCTCGAAATTAAGGGGCCC |
| | AGTACGTTGGACGGAAGAATTTCACTGCGTCAC |
| | CGGCGAGATTTAATCGGTCGCCCTTATTACTAC |
| | CA (SEQ ID NO: 889) |
| Robatumumab-BtsI-20-0 | CCCTTTAATCAGATGCGTCGTGCAAATGTGAG |
| | GTAGCAACGCAGTGTTTCGAACAATTTGCGAT |
| | ACCCGGCCCAGCCGGCCAGGCGCGAAGTCCA |
| | ACTGGTTCAGTCCGGGGGCGGCCTGGTGAAA |
| | CCCGGCGGCTCACTGCAACGCAAGCGAAAAC |
| | TACAAGGTCGCCCTTATTACTACCA (SEQ ID NO: 890) |
| Robatumumab-BtsI-20-1 | CCCTTTAATCAGATGCGTCGTGCAAATGTGAG |
| | GTAGCAACGCAGTGCTGGTGAAACCCGGCGG |
| | CTCCCTGAGGCTCTCATGCGCCGCCAGCGGAT |
| | TTACTTTTTCCTCATTTGCCATGCACTGGGTGA |
| | GGCAGGCACCAGGCACTGCAACGCAAGCGAA |
| | AACTACAAGGTCGCCCTTATTACTACCA |
| | (SEQ ID NO: 891) |
| Robatumumab-BtsI-20-2 | CCCTTTAATCAGATGCGTCGTGCAAATGTGAG |
| | GTAGCAACGCAGTGGGGTGAGGCAGGCACCA |
| | GGAAAAGGACTGGAGTGGATCAGCGTCATTG |
| | ATACAAGAGGTGCAACATATTACGCTGACAGC |
| | GTGAAGGGGAGATTTCACTGCAACGCAAGCG |
| | AAAACTACAAGGTCGCCCTTATTACTACCA |
| | (SEQ ID NO: 892) |
| Robatumumab-BtsI-20-3 | CCCTTTAATCAGATGCGTCGTGCAAATGTGAG |
| | GTAGCAACGCAGTGTGACAGCGTGAAGGGGA |
| | GATTTACAATTAGCCGCGATAACGCCAAGAAC |
| | TCCCTGTACCTGCAGATGAACTCCCTGCGGGC |
| | TGAAGACACAGCACTGCAACGCAAGCGAAAAC |
| | TACAAGGTCGCCCTTATTACTACCA |
| | (SEQ ID NO: 893) |
| Robatumumab-BtsI-20-4 | CCCTTTAATCAGATGCGTCGTGCAAATGTGAG |
| | GTAGCAACGCAGTGCCCTGCGGGCTGAAGAC |
| | ACAGCCGTGTACTATTGTGCAAGGCTGGGTAA |
| | TTTTTATTACGGCATGGACGTTTGGGGGCAGG |
| | GGACTACTGTGACACACTGCAACGCAAGCGAA |
| | AACTACAAGGTCGCCCTTATTACTACCA |
| | (SEQ ID NO: 894) |
| Robatumumab-BtsI-20-5 | CCCTTTAATCAGATGCGTCGTGCAAATGTGAG |
| | GTAGCAACGCAGTGGGGGCAGGGGACTACTG |
| | TGACAGTTTCCTCAGGGGGGAGCGGGGGGAG |
| | CGGGGGGGCTAGCGGCGCTGGCTCCGGAGG |
| | GGGAGAGATCGTCCTCACTGCAACGCAAGCG |
| | AAAACTACAAGGTCGCCCTTATTACTACCA |
| | (SEQ ID NO: 895) |
| Robatumumab-BtsI-20-6 | CCCTTTAATCAGATGCGTCGTGCAAATGTGAG |
| | GTAGCAACGCAGTGCCGGAGGGGGAGAGATC |
| | GTCCTGACACAGTCACCCGGGACTCTGTCTGT |
| | GAGCCCTGGCGAGAGAGCAACTCTGTCATGCA |
| | GGGCCAGCCACACTGCAACGCAAGCGAAAACT |
| | ACAAGGTCGCCCTTATTACTACCA |
| | (SEQ ID NO: 896) |
| Robatumumab-BtsI-20-7 | CCCTTTAATCAGATGCGTCGTGCAAATGTGAG |
| | GTAGCAACGCAGTGCTGTCATGCAGGGCCAG |
| | CCAAAGCATCGGCTCATCTCTGCACTGGTACC |
| | AGCAGAAACCCGGTCAGGCCCCACGCCTGCT |
| | GATCAAATATGCCAGCACTGCAACGCAAGCGA |
| | AAACTACAAGGTCGCCCTTATTACTACCA |
| | (SEQ ID NO: 897) |
| Robatumumab-BtsI-20-8 | CCCTTTAATCAGATGCGTCGTGCAAATGTGAG |
| | GTAGCAACGCAGTGACGCCTGCTGATCAAATA |
| | TGCCAGCCAGAGCCTGTCAGGCATTCCTGACA |
| | GATTTTCTGGGAGCGGATCAGGAACAGATTTC |
| | ACACTCACAATATCACTGCAACGCAAGCGAAAA |
| | CTACAAGGTCGCCCTTATTACTACCA |
| | (SEQ ID NO: 898) |
| Robatumumab-BtsI-20-9 | CCCTTTAATCAGATGCGTCGTGCAAATGTGAG |
| | GTAGCAACGCAGTGAGGAACAGATTTCACAC |
| | TCACAATATCCAGGCTGGAGCCCGAAGACTTC |
| | GCTGTCTACTACTGCCACCAGTCCAGCAGACT |
| | CCCTCACACCTTCGCACTGCAACGCAAGCGAA |
| | AACTACAAGGTCGCCCTTATTACTACCA (SEQ ID NO: 899) |
| Robatumumab-BtsI-20-10 | CCCTTTAATCAGATGCGTCGTGCAAATGTGAGGT |
| | AGCAACGCAGTGAGCAGACTCCCTCACACCTTC |
| | GGGCAAGGGACAAAGGTCGAAATTAAAGGGCCC |
| | GAGGCCCACTCGTATGATTATTCACTGCAACGCA |
| | AGCGAAAACTACAAGGTCGCCCTTATTACTACCA |
| | (SEQ ID NO: 900) |
| vedolizumab-BtsI-20-0 | CCCTTTAATCAGATGCGTCGAAAGTCAAAGTGCG |
| | TTTCGTGCAGTGTTAAGTGCACATTTCGTTTCGAG |
| | GCCCAGCCGGCCAGGCGCCAGGTGCAGCTGGTC |
| | CAATCTGGTGCAGAAGTGAAGAAACCTGGAGCTT |
| | CCGTGAACACTGCGGCTATGAGAGAGCAACACA |
| | GGTCGCCCTTATTACTACCA (SEQ ID NO: 901) |
| vedolizumab-BtsI-20-1 | CCCTTTAATCAGATGCGTCGAAAGTCAAAGTGCGT |
| | TTCGTGCAGTGAGAAACCTGGAGCTTCCGTGAAG |
| | GTGAGCTGTAAGGGGTCTGGGTATACCTTTACAA |
| | GCTATTGGATGCATTGGGTGAGACAAGCCCCCGG |
| | CCACTGCGGCTATGAGAGAGCAACACAGGTCGCC |
| | CTTATTACTACCA (SEQ ID NO: 902) |
| vedolizumab-BtsI-20-2 | CCCTTTAATCAGATGCGTCGAAAGTCAAAGTGCGT |
| | TTCGTGCAGTGGGTGAGACAAGCCCCCGGCCAGC |
| | GCCTCGAATGGATCGGGGAAATTGACCCTTCTGA |
| | ATCTAACACTAACTACAATCAGAAATTTAAGGGGA |
| | GAGTGACCACTGCGGCTATGAGAGAGCAACACAG |
| | GTCGCCCTTATTACTACCA (SEQ ID NO: 903) |
| vedolizumab-BtsI-20-3 | CCCTTTAATCAGATGCGTCGAAAGTCAAAGTGCGT |
| | TTCGTGCAGTGAATCAGAAATTTAAGGGGAGAGTG |
| | ACCCTGACCGTGGACATTTCAGCTTCTACTGCCTA |
| | CATGGAACTGTCCAGCCTGCGCTCTGAGGACACA |
| | GCCGCACTGCGGCTATGAGAGAGCAACACAGGTC |
| | GCCCTTATTACTACCA (SEQ ID NO: 904) |
| vedolizumab-BtsI-20-4 | CCCTTTAATCAGATGCGTCGAAAGTCAAAGTGCGT |
| | TTCGTGCAGTGTGCGCTCTGAGGACACAGCCGTT |
| | TACTATTGTGCCCGGGGCGGGTACGACGGTTGGG |
| | ACTATGCCATTGACTACTGGGGGCAAGGAACCCT |
| | GGTTACCACTGCGGCTATGAGAGAGCAACACAGG |
| | TCGCCCTTATTACTACCA (SEQ ID NO: 905) |
| vedolizumab-BtsI-20-5 | CCCTTTAATCAGATGCGTCGAAAGTCAAAGTGCGT |
| | TTCGTGCAGTGGGGGCAAGGAACCCTGGTTACAG |
| | TCTCAAGCGGTGGAAGCGCCGGTTCAGGTTCCTC |
| | AGGAGGGGCCTCAGGGTCAGGCGGAGATGTCGT |
| | GATGACCCACTGCGGCTATGAGAGAGCAACACAG |
| | GTCGCCCTTATTACTACCA (SEQ ID NO: 906) |
| vedolizumab-BtsI-20-6 | CCCTTTAATCAGATGCGTCGAAAGTCAAAGTGCGT |
| | TTCGTGCAGTGAGGCGGAGATGTCGTGATGACCC |
| | AATCTCCACTGAGCCTGCCTGTTACTCCCGGCGAG |
| | CCCGCATCAATCAGCTGCAGATCCTCTCAATCCCT |
| | GGCTCACTGCGGCTATGAGAGAGCAACACAGGTC |
| | GCCCTTATTACTACCA (SEQ ID NO: 907) |
| vedolizumab-BtsI-20-7 | CCCTTTAATCAGATGCGTCGAAAGTCAAAGTGCGT |
| | TTCGTGCAGTGTGCAGATCCTCTCAATCCCTGGCT |
| | AAGAGCTATGGAAATACCTACCTGTCATGGTACCT |
| | CCAGAAGCCTGGCCAATCACCCCAGCTGCTGATC |
| | TACGCACTGCGGCTATGAGAGAGCAACACAGGTC |
| | GCCCTTATTACTACCA (SEQ ID NO: 908) |
| vedolizumab-BtsI-20-8 | CCCTTTAATCAGATGCGTCGAAAGTCAAAGTGCGT |
| | TTCGTGCAGTGTCACCCCAGCTGCTGATCTACGGC |
| | ATTTCAAACAGATTCAGCGGCGTGCCTGATCGCTT |
| | CTCCGGTTCAGGGTCTGGTACTGATTTCACACTGA |
| | AGACACTGCGGCTATGAGAGAGCAACACAGGTCG |
| | CCCTTATTACTACCA (SEQ ID NO: 909) |
| vedolizumab-BtsI-20-9 | CCCTTTAATCAGATGCGTCGAAAGTCAAAGTGCGT |
| | TTCGTGCAGTGTCTGGTACTGATTTCACACTGAAG |
| | ATCTCTCGGGTGGAGGCAGAGGATGTGGGCGTCT |
| | ACTACTGTCTCCAGGGTACACACCAGCCATATACT |
| | TTCGGCACTGCGGCTATGAGAGAGCAACACAGGT |
| | CGCCCTTATTACTACCA (SEQ ID NO: 910) |
| vedolizumab-BtsI-20-10 | CCCTTTAATCAGATGCGTCGAAAGTCAAAGTGCGT |
| | TTCGTGCAGTGGTACACACCAGCCATATACTTTCG |
| | GGCAAGGGACAAAGGTCGAGATCAAGGGGCCCAC |
| | CGGTCAATTCTACCAACTTTCACTGCGGCTATGAGA |
| | GAGCAACACAGGTCGCCCTTATTACTACCA |
| | (SEQ ID NO: 911) |

Table 13 depicts oligonucleotides constructed on chips.

### REFERENCES

Leproust, E. M. et al. Synthesis of high-quality libraries of long (150mer) oligonucleotides by a novel depurination controlled process. Nucleic Acids Res. 38, 2522-2540 (2010).
Patwardhan, R. P. et al. High-resolution analysis of DNA regulatory elements by synthetic saturation mutagenesis. Nature Biotech. 27, 1173-1175 (2009).
Schlabach, M. R. et al. Synthetic design of strong promoters. P. Natl. Acad. Sci. USA 107, 2538-2543 (2010).
Li, J. B. et al. Multiplex padlock targeted sequencing reveals human hypermutable CpG variations. Genome Res. 19, 1606-1615 (2009).
Li, J. B. et al. Genome-wide identification of human RNA editing sites by parallel DNA capturing and sequencing. Science 324, 1210-1213 (2009).
Borovkov, A. Y. et al. High-quality gene assembly directly from unpurified mixtures of microarray-synthesized oligonucleotides. Nuc. Acids Res. E-publication (doi: 10.1093/nar/gkq677) (2010).
Borovkov et al., U.S. Patent Application No. 2009/0305233.
Church et al., U.S. Patent Application No. 2006/0014167.
Church et al., U.S. Patent Application No. 2006/0127920.
Church et al., U.S. Patent Application No. 2006/0194214.
Church et al., U.S. Patent Application No. 2006/0281113.
Ai, H et al. (2006) Biochem. J. 400:531.
Griesbeck et al. (2001) J. Biol. Chem. 276:29188.
Shaner et al. (2008) Nat. Methods 5:545.
Burland (1999) Meth. Mol. Biol. 132, 71.

### Example II

### Methods Summary

Reanalysis of OLS Pool Error Rates

Church et al., U.S. Patent Application No. A previously published data set was re-analyzed to determine sequencing error rates (Slater and Birney (2005) BMC Bioinformatics 6:31). Briefly, the dataset was derived from high-throughput sequencing using the Illumina Genome Analyzer platform of a 53,777 150mer OLS pool. Two sequencing runs were performed; the first before any amplification, and the second after two rounds of ten cycles of PCR (20 cycles total). As the previous analyses were mostly looking for distribution effects, the existing data as re-analyzed to get an estimate of error rates pre- and post-PCR amplification. The dataset was realigned using Exonerate to allow for gapped alignments and analysis of indels (Li H. Maq: mapping and assembly with qualities, Welcome Trust Sanger Institute (2010), available at Worldwide Website: maq.sourceforge.net). Specifically, an affine local alignment model that is equivalent to the classic Smith-Waterman-Gotoh alignment was used having a gap extension penalty of −5. The full refine option was used to allow for dynamic programming based optimization of the alignment. These reads were solely mapped on base calls by the Illumina platform. These alignments were used to count mismatches, deletions, and insertions as compared to the designed sequences. However, since base-calling can be more error prone on next generation platforms than traditional Sanger-based approaches, the results were filtered based only on high-quality base-calls (Phred scores of 30 or above or >99.9% accuracy). This was accomplished by converting Illumina quality scores to Phred values using the Maq utility sol2sanger (Id.) and only using statistics from base calls of Phred 30 or higher. All error rate analysis scripts were implemented in Python. While this method provided an estimate for error rates, without intending to be bound by scientific theory, unmapped reads may have higher error rates and thus underestimating the total average error rate. In addition, base-calling errors might still overestimate the error rate. Finally, using only high-quality base calls, which usually occur only in the first 10 bases of a read, might only reflect error rates on the 5′ end of the synthesized oligonucleotide.

### Design and Synthesis of OLS Pools

The 13,000 oligos in the first OLS library (“OLS Pool 1”) were broken up into 12 separately amplifiable subpools (“assembly subpools). Each assembly subpool was defined by unique 20 bp priming sites that flanked each of the oligos in the pool. The priming sites were designed to minimize amplification of oligos not in the particular assembly subpool. This was done by designing set of orthogonal 20-mers (“assembly-specific primers”) using a set of 240,000 orthogonal 25-mers designed by Xu et al. ((2009) Proc. Natl. Acad. Sci. USA 106:2289) as a seed. From these sequences 20-mers with 3′ sequence ending in thymidine or ‘GATC’ were selected for the forward and reverse primers respectively. Melting temperatures between 62-64° C. and low primer secondary structure of the primers were screened. After the additional filtering, 12 pairs of forward and reverse primers were chosen to be the assembly-specific primers. The 13,000 oligos in the second OLS library (“OLS Pool 2”) were broken up into 11 subpools corresponding to 11 sets of up to 96 assemblies (“plate subpools”), which were further divided into a total of 836 assembly subpools. A new set of orthogonal primers was designed similarly to the previous set (without the GATC and thymidine constraints) but further filtered to remove Type IIS restriction sites, secondary structure, primer dimers, and self-dimers. The final set of primer pairs was distributed among the plate-specific primers, assembly-specific primers, and construction primers

OLS pools were synthesized by Agilent Technologies. Costs of OLS pools were a function of the number of unique oligos synthesized and of the length of the oligos (less than $0.01 per final assembled base-pair for all scales used herein). OLS Pools 1 and 2 were independently synthesized, cleaved, and delivered as lyophilized, approximately 1-10 picomole pools.

### Amplification and Processing of OLS Subpools

Lyophilized DNA from OLS Pools 1 and 2 were resuspended in 500 μL TE. Assembly subpools were amplified from 1 μL of OLS Pool 1 in a 50 μL qPCR reaction using the KAPA SYBR FAST qPCR kit (Kapa Biosystems). A secondary 20 mL PCR amplification using Taq polymerase was performed from the primary amplification product. The barcode primer sites were removed using a technique previously described (Porreca et al. (2007) Nat. Methods 4:931). In brief, the forward primers contained a phosphorothioate bond at the 5′ end and the last nucleotide on the 3′ end was a deoxyuridine; the reverse primers contained a DpnII recognition site (‘GATC’) at the 3′ end and a phosphorylated 5′ end. PCR amplification was followed by λ exonuclease digestion of 5′ phosphorylated strands, hybridization of the 3′ primer site to its complement, and cleavage of the 5′ and 3′ primer sites using USER enzyme mix and DpnII (New England Biolabs), respectively. Plate subpools were amplified from 1 μL of OLS Pool 2 in 50 μL Phusion polymerase PCR reactions. Assembly subpools were amplified from the plate subpools by 100 μL Phusion polymerase PCR reactions. A BtsI digest removed the forward and reverse primer sites.

### Assembly of Fluorescent Proteins

GFPmut3 (Carmack et al. (1996) Gene 173:33) was assembled from the OLS Pool 1 assembly subpools by PCR. The GFP43 and GFP35 subpools were designed such there was full overlap between neighboring oligos during assembly, with average overlaps of 43 bp and 35 bp for GFP43 and GFP35, respectively. For the first set of assemblies, 330 pg of the GF43 subpool or 40 pg of the GFP35 subpool were used per 20 μL Phusion polymerase PCR assembly. The full-length product was gel-isolated, amplified using Phusion polymerase, and cloned into pZE21 after a HindIII/KpnI digest. The second set of assemblies was built using a similar procedure, except that the assembly PCR used 170 pg or 190 pg of GFP43 and GFP35 subpools, respectively; and the gel-isolated product was not re-amplified prior to cloning.

Oligonucleotides for mTFP1, mCitrine, and mApple were designed such that there was on average a 20 bp overlap between adjacent oligonucleotides. The proteins were built from OLS Pool 2 assembly subpools by first performing a KOD polymerase pre-assembly reaction that was done in the absence of construction primers followed by a KOD polymerase assembly PCR in which the construction primers were included. ErrASE error correction was then performed on aliquots of the synthesis products following the manufacturer's instructions. The assembled product was digested with HindIII and KpnI and cloned into pZE21. Sequencing of clones was performed by Beckman Coulter Genomics.

### ErrASE

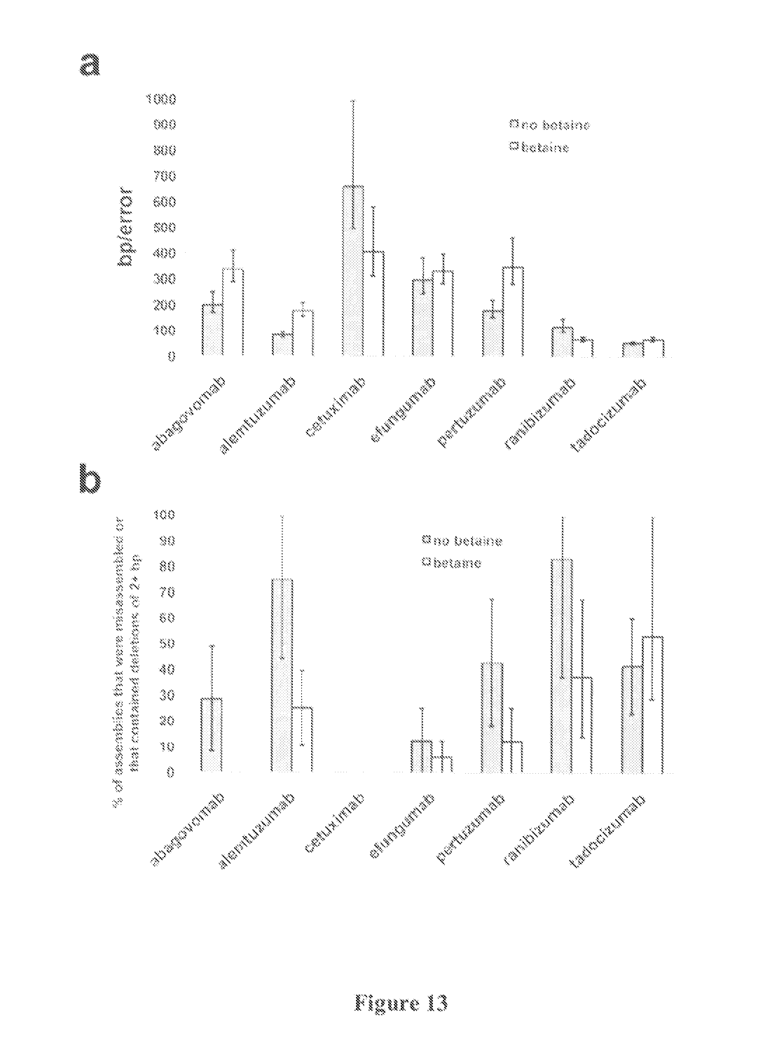

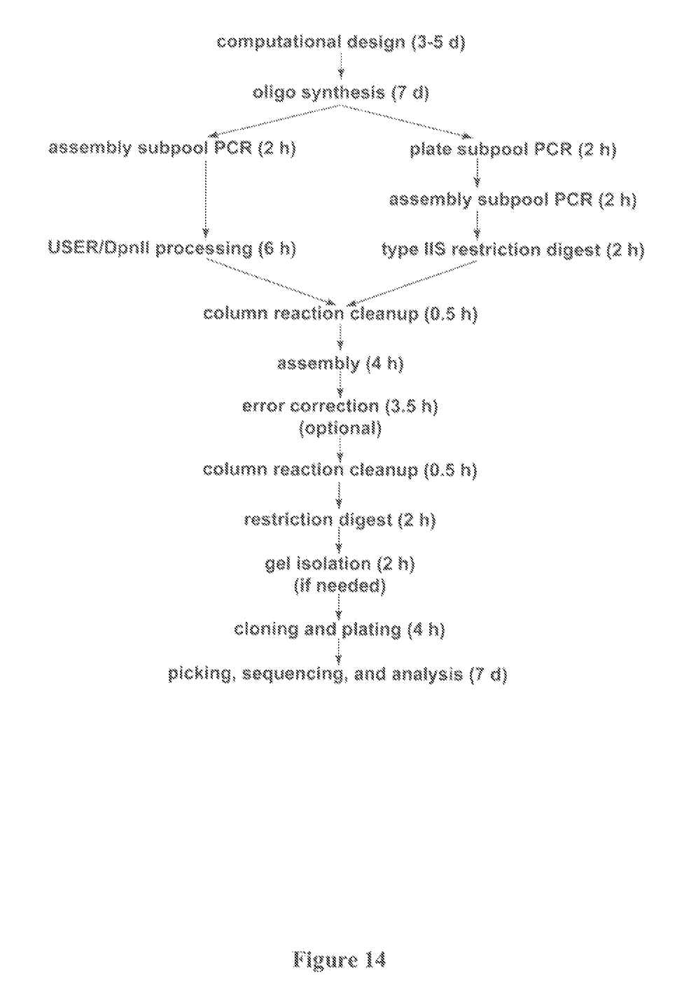

Six aliquots of 10-50 ng of each assembled gene was added to 10 μL of PCR buffer (the effects of including betaine in the buffer were also examined, see FIG. 13). Heteroduplexes were formed by denaturing at 95° C. and slowly cooling to room temperature. Each aliquot was then used to resuspend six different lyophilized ErrASE mixtures of increasing stringency provided by the manufacturer. After a 1-2 hour room temperature incubation, the assemblies were re-amplified and visualized on an agarose gel. Of the reactions that resulted in a correctly-sized band, the one that used the most stringent ErrASE protocol was selected for cloning.

### Flow Cytometry

Fluorescent cell fractions of the cloned libraries of assembly products were quantified using a BD LSR Fortessa flow cytometer either a 488 nm laser with a 530 nm filter (30 nm bandpass) or a 561 nm laser with a 610 nm filter (20 nm bandpass).

### Synthesis of Antibodies

125 ng of each antibody assembly pool was pre-assembled in 20 μL KOD pre-assembly reactions. Nine amplification protocols were then tested for the ability to amplify the 42 antibody pre-assemblies into full-length genes. An attempt was made to clone 8 constructs from the best assembly protocol (afutuzumab, efungumab, ibalizumab, oportuzumab, panobacumab, robatumumab, ustekinumab, and vedolizumab; see Supplementary FIG. 12A and Table 3). The eight assemblies were error-corrected using ErrASE, gel-isolated, re-amplified using Phusion polymerase, gel-isolated again, and cloned into pSecTag2A after an ApaI/SfiI digest. Sequencing was performed by Genewiz. All but oportuzumab cloned successfully. The experiment was then repeated, increasing the amount of assembly pool DNA in the pre-assembly reaction to 400 ng. A different set of 8 constructs was selected from this second set of assemblies for cloning (abagovomab, alemtuzumab, ranibizumab, cetuximab, efungumab, pertuzumab, tadocizumab, and trastuzumab; see FIG. 2D and Table 3). Using the same methods as with the first set of cloned antibodies, this second set was error-corrected, gel-isolated, cloned, and sequenced.

### Example III

### Detailed Methods

### OLS Pool Overall Design

The first OLS library (OLS Pools 1) consisted of 12 separately amplifiable assembly subpools. Of the 13,000 oligonucleotides (oligos) that were made in OLS Pool 1, there were two subpools, GFP43 and GFP35, that were designed to each synthesize the mut3 variant of GFP (GFPmut3b) (Cormack et al. (1996) Gene 173:33). GFP43 consisted of 18 oligos while GFP35 had 22. The individual subpools assembled into 779 bp constructs, of which 719 bp could be cloned and verified downstream after restriction digest. Two other subpools were used as amplification controls (Control 1 and 2) and contained 10 and 5 130mers, respectively. The remaining 12,945 OLS Pool 1 oligos consisted of 130mers having homology to the E. coli genome that was split into 8 separate amplification subpools. The OLS array was synthesized, processed from the chip, and delivered as an approximately 1-10 pmol lyophilized pool of oligos by Agilent Technologies (Carlsbad, Calif.).

### Design of GFPmut3 Assembly Subpools

Forward and reverse GFPmut3 assembly oligos were designed to have complete overlap, as well as a bridging oligonucleotide to allow for tests with both circular ligation assembly and PCR assembly protocols (Bang and Church (2008) Nat. Methods 5:37). The overlap lengths were 43 bp and 35 bp for GFP43 and GFP35, respectively. An algorithm that automatically splits the constructed sequences into adjacent annealing segments of similar melting temperatures was developed that was loosely based on the Gene2Oligo design method (Rouillard et al. (2004) Nucleic Acids Res. 32:W176). Briefly, the algorithm first adds random DNA sequence on the ends of the constructed gene to allow for leeway on the first and last annealing segment. Next, the algorithm enumerates all possible overlap regions for the gene to be constructed that fall within a certain length range and sorts them into bins based on their start position. The mean melting temperature is calculated for all overlap regions, and regions that do not fall within a defined temperature deviation are removed. Bins are sorted in order based on minimal deviation from the mean melting temperature. The program then recursively attempts to construct the gene from left to right by picking the first region from the top of the list. If a particular position has no annealing regions (no regions match the melting temperature), the program backtracks and picks the next valid annealing region and tries again. Once a valid set of annealing regions is designed, the algorithm designs oligos that span two adjacent annealing regions alternating between the sense and antisense strands. Finally, a bridging oligo that spans the first and last segment is designed. The requirement of a bridging oligo necessitates that an even number of annealing regions are designed and the algorithm takes this into account.

The GFP43 subpool was designed using a seed overlap region size of 43, size variability of ±2, and a temperature variability of 4.5° C. The resultant designs had 18 oligos with a mean melting temperature of 72.5° C. with a 1.8° C. average deviation. The GFP35 subpool was designed using a seed overlap region size of 35, size variability of ±4, and temperature variability of 3° C. The resultant designs had 22 oligos with a mean melting temperature of 69.6° C. with a 1.6° C. average deviation. Finally, a pool of oligos, GFP20, were designed that were made using column-based synthesis and which could construct GFPmut3. The GFP20 design used a seed overlap region size of 20, size variability of 3, and a temperature variability of 5° C. The resultant designs had 40 oligonucleotides with a mean melting temperature of 56.3° C. with a 1.0° C. average deviation.

### Design of Subpool Assembly-Specific Primers

There was a total of 12 assembly subpools designed for OLS Pool 1. Orthogonal primers were selected from a set of 240,000 previously designed orthogonal 25mer barcodes designed for yeast genomic hybridization studies (Xu et al. (2009) Proc. Natl. Acad. Sci. USA 106:2289). Briefly, each barcode was searched for reverse primers for 20mers that end in ‘GATC’. Forward primers were selected from barcode primers that end in ‘T’. Both forward and reverse primer sets were screened for melting temperatures between 62° C. and 64° C. calculated using the nearest neighbor method (SantaLucia (1998) Proc. Natl. Acad. Sci. USA 95:1460; SantaLucia and Hicks (2004) Ann. Rev. Bioph. Biom. 33:415). Primers were then screened by BLAT for hits (tilesize=6, stepsize=1, minMatch=1) against one another, as well as against the E. coli genome (Kent (2002) Genome Res. 12:656). Primers with greater than 1 self-hit, or 3 E. coli genome hits were removed. Secondary structures were then calculated using UNAFold, and any primers containing folding energies less than 0 kcal/mol were removed (Markham and Zuker (2008) Meth. Mol. Biol. 453:3). Primers pairs were then screened using MultiPLX to obtain a group of orthogonal primers, from which 12 primers were chosen to be assembly-specific primers (Kaplinski et al. (2005) Bioinformatics 21:1701). All scripts were written in Python and used several BioPython utilities (Cock (2009) Bioinformatics 25:1422).

### Assembly Subpool Amplification

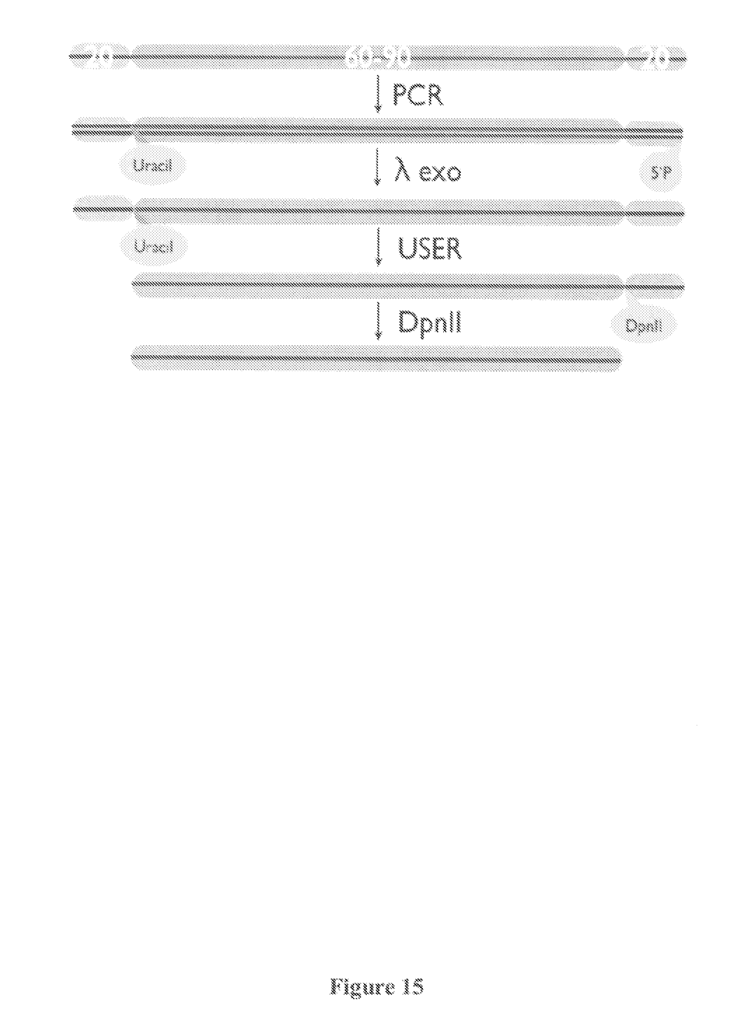

Lyophilized DNA recovered from OLS Pool 1 (approximately 1 pmol total DNA) was resuspended in 500 μL TE Buffer. Each of the four assembly subpools (GFP43, GFP35, Control 1, and Control 2) were amplified in 50 μL reactions using the KAPAprep protocol (all italicized PCR protocols are named and described in the PCR protocol Table at the end of this supplement) with the appropriate assembly-specific primers and 1 μL of the reconstituted OLS Pool 1. These PCR reactions were monitored by real-time PCR and were stopped before reaching plateau fluorescence levels to prevent over-amplification (between 35-45 cycles). Two replicates were pooled and purified using QIAquick PCR Purification Kit (QIAGEN Inc., Valencia, Calif.). The resultant subpools were size verified and quantified on gels to give between 20 and 35 ng/μL of DNA in 30 μL total. 20 μL of each subpool was re-amplified in 20 mL total volume spread split into two 96-well plates using the TaqPrep protocol with chemically modified assembly-specific primers (see FIG. 15 for details). Samples were spun down in Amicon Ultra-15 mL Centrifugal Filter with Ultracel-10 membrane at 4,000 g in a swinging bucket rotor, washed in 13 mL TE Buffer, and recovered into 350 μL total volume. 40 μL of 1 AU/mL QIAGEN Protease was added to each sample, and shaken at 800 rpm in a Thermomixer R (Eppendorf AG, Hamburg Germany) at 37° C. for 40 min, and then 20 min at 70° C. to heat inactive. 70 μL of RapidClean Protein Removal Resin (Advantsa, Menlo Park Calif.) was added, mixed for 15 seconds, and spun down at 24,000 g in an Eppendorf Centrifuge 5424 for 5 minutes, and the supernatant was removed. The supernatant was rewashed in water in an Amicon Ultra-0.5 mL Centrifugal Filter with Ultracel-10 membrane and volume adjusted to 450 μL.

### Assembly Subpool Processing

Purified samples from above were treated with lambda exonuclease (Enzymatics) to make them single stranded. 445 μL of the filtrate, 150 μL 10× lambda exonuclease buffer, 805 μL water, and 100 μL lambda exonuclease was incubated at 37° C. for 40 minutes and 20° C. for 20 minutes and shaken at 800 rpm in a Thermomixer R. Samples were spun down in Amicon Ultra-0.5 mL Centrifugal Filter with Ultracel-3 membrane and washed with water and recovered in 350 μL water. 300 μL of each sample was then processed with 1250 U of DpnII (New England Biolabs, Ipswich, Mass.), 125 U USER Enzyme (New England Biolabs), and 3 nanomoles of the guide oligo (the reverse subpool amplification primer without a 5′ phosphate) in 2.5 mL of 1× DpnII buffer, and incubated at 800 rpm at 37° C. Samples were then filtered in an Amicon Ultra-15 mL 3 kDa filter, washed first with 2 mL TE, and then with 4 mL water. The ssDNA product was recovered in 130 μL for control subpools 1 and 2, and 50 μL for GFP43 and GFP35 assembly subpools.

### First OLS Pool 1 Assemblies

### Assembly

GFPmut3b was assembled from column-synthesized oligos (IDT, Coralville, Iowa) by amplifying 1 μL of a pool of 19 reverse oligos (1.05 μM each) and 20 forward oligos (1 μM each) in a 20 μL reaction using the Phu1 protocol with the forward and reverse construction primers (GFPfwd and GFPrev, IDT). The reaction was heated to 98° C. for 30 seconds, followed by 30 cycles of 98° C. for 5 seconds, 51° C. for 10 seconds, and 72° for 30 seconds. This was followed by a final extension of 72° C. for 10 minutes.

The concentrations of the assembly subpools were determined using a Nanodrop 2000c spectrophotometer (Thermo Scientific, Wilmington, Del.), as were all measurements of DNA concentration described in the methods infra. GFP43 and GFP35 assembly subpools were assembled into GFPmut3 by amplifying 330 pg of GFP43 or 40 pg of GFP35 in a 20 μL reaction using the Phu1 protocol with the forward and reverse construction primers (GFPfwd and GFPrev). The full-length products from both assemblies were isolated by running 18 μL of the assembly PCR on four lanes of a 2% EX E-Gel (Invitrogen, Carlsbad, Calif.) and extracting the DNA using a QIAquick Gel Extraction Kit (QIAGEN). This yielded 4 ng and 6 ng of GFPmut3 built from subpools GFP43 and GFP35, respectively—both in 50 μL EB buffer (10 mM Tris-Cl, pH 8.5). 1 μL of the gel-isolated DNA was amplified in 20 μL reactions using the Phu1 protocol. Each gel-isolated assembly was amplified in 24 different PCR reactions. The amplification products were cleaned up using a QIAquick PCR Purification Kit.

### Cloning

For screening all fluorescent proteins, the expression plasmid pZE21 (Lutz and Bujard (1997) Nucleic Acids Res. 25:1203) was used. 10-beta (New England Biolabs) E. coli cells transformed with the plasmid were streaked out on LB agar plates containing 50 μg/mL kanamycin. A single colony was then grown for 17 hr in 2 mL LB with 50 μg/mL kanamycin and thereafter kept at 4° C. for less than 60 hours. This culture was back-diluted by adding 100 μL to 100 mL of fresh LB/kanamycin medium and grown for 17 hours at 37° C. and stored at 4° C. for 3 hours. The plasmid was isolated using QIAprep Spin Miniprep Kit (QIAGEN).

GFPmut3b was amplified from 9-10 ng of pZE21G (Isaacs et al. (2004) Nat. Biotechnol. 22:841) in 50 μL reactions using the Phu2 protocol with the primers GFPfwd2 and GFPrev2. The products were cleaned up using a QIAquick PCR Purification Kit. To generate the stock of control GFPmut3 used in all subsequent fluorescent protein cloning experiments, 10-20 ng of the amplified product was re-amplified in 50 μL reactions using the Phut protocol (except that dNTPs from Kapa Biosystems were used), again using primers GFPfwd2 and GFPrev2. The products were cleaned up using a QIAquick PCR Purification Kit.

4.9 μg of GFP43 assembly, 5.8 μg of GFP35 assembly, 4.2 μg of GFP20 assembly, 2.7 μg of the GFP control, and 2.7 μg of pZE21 were digested in separate 50 μL reactions that consisted of 1× NEBuffer 2 (500 mM NaCl, 100 mM Tris-HCl, 100 mM MgCl₂, 10 mM dithiothreitol, pH 7.9; New England Biolabs), 100 ng/μL bovine serum albumin (New England Biolabs), 0.4 units/μL of HindIII (New England Biolabs), and 0.54 units/μL KpnI (New England Biolabs). The assemblies were digested at 37° C. for 3 h while shaking at 800 rpm in a Thermomixer R. After GFP control and pZE21 were digested for 2.5 hours at 37° C., 1 μL of 20 units/μL DpnI (New England Biolabs) was added to the GFP control digests and 1 μL of 5 units/μL Antarctic phosphatase (New England Biolabs) and 5.6 μL 10× Antarctic phosphatase buffer (New England Biolabs) were added to the pZE21 digests. The GFP control and plasmid were kept at 37° C. for 30 minutes while shaking at 800 rpm in a Thermomixer R. The enzymes in all reactions were heat inactivated at 65° C. for 20 minutes while shaking at 800 rpm in a Thermomixer R. The products were cleaned up using a QIAquick PCR Purification Kit.

HindIII/KpnI digested assemblies from GFP43, GFP35 or GFP20 were cloned as follows. 180 ng of one of the inserts and 40 ng of HindIII/KpnI digested pZE21 were diluted in 8.5 μL water. 1 μL of 10×T4 ligase buffer (New England Biolabs) was added, and the reaction was heated to 37° C. for 5 minutes. The reaction was brought down to room temperature, and 0.5 μL of 400 units/μL of T4 DNA ligase (New England Biolabs) was rapidly added. The ligation was then allowed to proceed for 10 minutes at 25° C. The enzyme was heat-inactivated for 15-25 minutes at 65° C. All thermal steps were conducted with shaking at 800 rpm in a Thermomixer R. A 25 nm mixed cellulose ester membrane (Millipore, Billerica, Mass.) was used to dialyze the ligation product against a 1.000-fold greater volume of water for 5-15 min. 2 μL of the dialyzed ligation product was added to 50 μL freshly thawed NEB 10-beta electrocompetent E. coli cells (New England Biolabs), and the mixture was briefly incubated on ice. Electroporation was performed with one pulse of 1.8 kV using Gene Pulser cuvettes with a 0.1 cm electrode gap (Bio-Rad, Hercules, Calif.) in a MicroPulser (BioRad). The cells were suspend in 1 mL LB medium and incubated at 37° C. for 70 minutes. A fraction of each culture was then plated onto 50 μg/mL kanamycin LB agar plates and grown overnight at 37° C. The 1 mL non-selective culture was stored at 4° C. for 23 hours, after which 1 μL was inoculated into 1 mL of 50 μg/mL LB that was subsequently grown overnight at 37° C.

### Flow Cytometry

For each cloning reaction, 10 μL of the overnight culture in selective medium was added to 1 mL 50 kanamycin and grown at 37° C. for 1-2 hours. The fluorescent cell fraction was then quantified using a BD LSRFortessa flow cytometer (BD Biosciences, San Jose, Calif.) using a 488 nm blue laser and a FITC detector (530 nm filter with 30 nm bandpass).

### Sequencing

Colonies were randomly picked from selective agar cultures corresponding to each ligation reaction. Each colony was inoculated into 200 μL of 50 μg/mL LB and grown overnight at 32° C. Each 200 μL overnight culture was split into two 100 μL aliquots, and 100 μL 30% glycerol in water was added to each aliquot. The stocks were immediately placed into −80° C. storage. Dideoxy sequencing of one of the two 200 μL glycerol stocks was performed by Beckman Coulter Genomics (Danvers, Mass.) using the following primers: forward-5′ ATAAAAATAGGCGTATCACGAGGC (SEQ ID NO:912); reverse-5′ CGGCGGATTTGTCCTACTCAG (SEQ ID NO:913). The second glycerol stock was kept to make possible the recovery of sequenced clones.

### Second OLS Pool 1 Assemblies

### Assembly

170 pg of the GFP43 and 190 pg of the GFP35 assembly subpools were assembled into GFPmut3 in separate 20 μL reactions using the Phu1 protocol with the construction primers (GFPfwd and GFPrev). The full length products were isolated from a 2% agarose gel using a QIAquick Gel Extraction Kit, with the product of 23 GFP43 assembly reactions concentrated into 50 μL EB buffer, and 70 GFP35 assembly reactions concentrated into 135 μL EB buffer. 10 μL of the assembly products were then digested in 50 μL KpnI/HindIII reactions identical to the one described during the cloning of the first set OLS Pool 1 assemblies (except for the lack of the 65° C. heat inactivation step). The digested products were cleaned up using a MinElute PCR Purification Kit (QIAGEN).

### Cloning

Using a 2% EX E-Gel and a quantitative DNA ladder, the concentrations of GFPmut3 assemblies from GFP43 and GFP35 were determined to be 14 ng/4 and 35 ng/μL, respectively. The PCR-amplified KpnI/HindIII-digested 40 ng/μL GFPmut3 stock prepared during the first assembly experiment was used as a positive control, and the 180 ng/μL stock of KpnI/HindIII-digested pZE21 prepared during the same experiment was used as the cloning vector. Electrocompetent E. coli cells were prepared by concentrating a 2 L culture of NEB 5-alpha cells (New England Biolabs) into 50 mL of water.

14 ng of GFP43 and 35 ng of GFP35 were each added to 180 ng of vector and were ligated in a 10 μL T4 ligase reaction the products of which were electroporated into NEB 5-alpha cells following the protocol described in the cloning of the first OLS Chip 1 constructs. After an outgrowth of 37° C. for 70 min, 100 μL of the culture was diluted into 900 μL of LB with 50 μL/mL kanamycin, and another fraction was plated onto 50 μg/mL kanamycin LB agar plates. Both the plated cells and the cells in liquid culture were grown overnight at 37° C.

### Flow Cytometry

20 μL of each overnight culture of the non-error corrected constructs was diluted into 2 mL 50 μg/μL kanamycin LB and grown at 37° C. for 2 hours. The fluorescent cell fraction was then quantified using a BD LSRFortessa.

### Sequencing

Random clones were grown overnight in LB, made into glycerol stocks, and sequenced by Beckman Coulter Genomics following the protocol described in the sequencing of the first OLS Chip 1 constructs.

### Error Correction

HindIII/DpnI-digested assemblies (840 pg of GFP43 and 380 pg of GFP35) were amplified in separate 20 μL reactions following the Phu3 protocol and using the primers GFPfwd3 and GFPrev3. Each assembly was amplified in four 20 μL reactions, which were subsequently pooled and cleaned up in a single QIAquick PCR Purification Kit column.

Error correction using ErrASE (Novici Biotech, Vacaville, Calif.) was performed using a slight variation of the manufacturer's protocol. In brief, either 2.8-2.9 mg of GFP protein assembly were added to separate 50 μL reactions consisting of 0.9× Phusion HF buffer with 180 μM dNTPs (Enzymatics). Each reaction was heated to 98° C. for 1 minute, cooled to 0° C. for 5 minutes, kept at 37° C. for 5 minutes, and subsequently stored and handled at 4° C. 10 μL of the reaction was then added to each of first five of the six decreasingly stringent ErrASE reactions, and the mix was incubated at 25° C. for 1 hour while shaking at 800 rpm in a Thermomix R. 2 μL of the ErrASE reactions were then re-amplified in 50 μL reactions using the Phu3 protocol with the primers GFPfwd3 and GFPrev3.

### Post-ErrASE Cloning, Flow Cytometry and Sequencing

The highest stringency ErrASE reaction that resulted in a PCR product (#2 for both assemblies) was cleaned up using a QIAquick PCR Purification Kit. 260 ng of GFP43 and 960 ng of GFP35 were digested in 40 μL reactions with 4 μL NEBuffer 2, 0.4 μL bovine serum albumin, 0.5 μL HindIII (20 units/4), 1.4 μL KpnI (10 units/4), and water. The error-corrected constructs were digested at 37° C. for 2 h while shaking at 800 rpm in a Thermomixer R. Although electrophoresis on an agarose gel detected only the single, correct band, the constructs were gel isolated using a QIAquick Gel Extraction Kit in order to remove any undetected misassemblies.

20 ng of pZE21 and either 35 ng of gel-isolated GFP43, 65 ng of gel-isolated GFP35, or 70 ng of control GFP (same prep as was used during the previous ligation experiments) were diluted in 8.5 μL water. The DNA was then ligated in a 10 μL T4 ligase reaction the products of which were electroporated into NEB 5-alpha cells following the protocol described in the cloning of the first OLS Chip 1 constructs. After an outgrowth of 37° C. for 65 minutes, 400 μL of the culture was diluted into 2 mL of LB with 50 μL/mL kanamycin, and another fraction was plated onto 50 μg/mL kanamycin LB agar plates. Both the plated cells and the cells in liquid culture were grown overnight at 37° C.

For each overnight culture, 5 μL was diluted into 500 μL 50 kanamycin LB and grown at 37° C. for 1.5 hour. The fluorescent cell fraction was then quantified using the BD LSRFortessa flow cytometer. The fluorescent fraction of each overnight culture was measured across 7-8 technical replicates. The data from one replicate per culture was removed from the analysis due to obvious fluidics-mediated sample carryover between the last wells and the first wells of the different experiments conditions.

Random clones were grown overnight in LB, made into glycerol stocks, and sequenced by Beckman Coulter Genomics following the protocol described in the sequencing of the first OLS Chip 1 constructs (except that the overnight culture was performed at 37° C.).

### OLS Pool 2

### Overall Design

The pool of oligos from the second OLS chip (OLS Pool 2) was designed specifically for gene synthesis applications. In total, the chip encoded 12,998 oligonucleotides encoding 2,456,706 nucleotides of synthetic DNA. OLS Pool 2 was split into 11 plate subpools, which were further divided into a total of 836 assembly subpools. The 836 potential assemblies encoded 869,125 bp of DNA after all primer processing steps.

### Redesign of Orthogonal Primers

Initial experiments began by scaling up the primer design method for OLS Pool 1 to allow for the design of 3,000 orthogonal primer pairs. The same set of 240,000 orthogonal barcodes as in OLS Pool 1 was used. In order to facilitate current and possible future downstream cloning and processing steps, primers containing restriction enzyme recognitions sites to the following enzymes were removed: AatII, BsaI, BsmBI, SapL BsrDI, BtsI, Earl, BspQI, BbsI, BspMI, BfuAI, NmeAIII, BamHI, NotI, EcoRI, KpnI, HindIII, XbaI, SpeI, PstI, Pad, and SbfI. Then, all primers with melting temperature below 60° C. and above 64° C. were removed to facilitate melting temperature matching of forward and reverse primers. Finally, an algorithm was implemented that screens primers for primer dimer formation that follows the AutoDimer program (Vallone and Butler (2004) BioTechniques 37:226), though giving double weight to the terminal 10 bases on the 3′ end. All primers with a score greater than 3 were removed. After these screens, 155,608 primers remained. A BLAST library was constructed of all synthesized genes on the chip (except the fluorescent proteins), each oligo was screened against the library using BLAT (tileSize=6, stepSize=1, minMatch=2, maxGap=4), and any primers with hits were removed leaving 70,498 primers. A second BLAST library was constructed from the remaining primers, and a network elimination algorithm as described in the orthogonal barcode paper was applied (tileSize=6, stepSize=1, −minMatch=1, maxGap=4)(Li and Elledge (2007) Nat. Methods 4:251). This resulted in 8275 remaining primers, which were screened for formation of secondary structure (ΔG greater than −2). Finally, the 7738 remaining primers were aligned using clustalw2 (default options for DNA(slow)), clustered, and a phylogenetic tree was generated. This tree was traversed to find 200 nodes that were distant from one another and contained at least 30 primers each. Then, one primer from each batch was chosen. Primers were sorted on melting temperature, and then paired provided that they pass a primer dimer test (filtered dimers with a score greater than 4). The final output was a set of 3,000 pairs of orthogonal primers, grouped in sets of 100. The first set was reserved as plate-specific primers (skpp1-100), the second set was reserved for construction primers (skpp101-200), and each remaining set was used in order for assembly-specific primers.

### Construct Designs

Automated algorithms were written to split constructs into oligonucleotide segments with partial overlaps to allow for stringent PCR assembly. Given a desired overlap size, allowable leeway on the size and position of the overlaps, and a melting temperature range, and Type IIs restriction enzyme site, the program automates the process of turning full-length gene constructs into oligonucleotides to be synthesized on the OLS platform. Briefly, the algorithm starts by padding the sequence with the proper construction primers. Then, the construct is evenly divided among the number of necessary oligonucleotides to construct the whole sequence, automatically determining the starting overlap positions. These overlap positions are screened for melting temperature falling within the defined length range, secondary structure formation ((AG greater than −3), and self dimer formation (score greater than 3) (see orthogonal primer design section). If these conditions are not met, the overlap lengths and positions are progressively varied and rechecked according to the predefined boundaries set at the beginning of the run. Once an overlap set is found that satisfies all the conditions, the final oligonucleotides are defined, and then flanked with the proper Type IIs restriction sites followed by the assembly-specific and plate-specific primer sequences. All sequences are rechecked for proper restriction enzyme cutting to make sure additional restriction sites were not added by adding primer sequences (in which case, the program pads with arbitrary sequence to remove the restriction site).

64 assemblies were designed that encoded three codon-optimized fluorescent proteins, mTFP114, mCitrine15, and mApple16. Codon-optimization was done using a custom algorithm that randomly assigned codons weighted to their natural frequencies in the E. coli genome as defined by the Kazusa Codon Usage Database (Worldwide Web Site: kazusa.or.jp/codon/). Each protein (mApple was synthesized twice for each of these conditions) was fed through the algorithm varying overlap length (15,18,22,25 bp average overlaps) and fixing Type IIs cutters (BtsI and BspQI), or varying Type IIs restriction enzyme sites (BtsI, BspQI, BsrDI, EarI, BsaI, BsmBI, SapI, BbsI) and fixing average overlap lengths. The allowable melting temperature ranges were: 15 bp overlap—50-55° C.; 18 bp overlap—53-58° C.; 20 bp overlap—58-62° C.; 22 bp overlap—58-65° C.; 25 bp overlap—65-72° C. In addition, the overlap length leeway was set to ±3, and position leeway to ±5. These 64 assemblies were designed to be amplified together using a single plate-specific amplification, and then individually using assembly-specific primers. The assembly of one of the conditions, which is from the BtsI with 20 bp overlap, is illustrated further herein.

The 42 antibody assemblies were designed as described in the Examples above (V region sequences were obtained from the IMGT database (Lefranc et al. (2009) Nucleic Acids Res. 37:D1006). Amino acid sequences for the antibodies were codon optimized for human expression using the same algorithm and settings as the fluorescent protein designs in the 20 bp overlap, BtsI restriction enzyme condition. The segments of the 42 antibodies were flanked by different plate-specific pool primers than the fluorescent proteins, and individually addressable using assembly-specific primers.

Fluorescent Proteins from OLS Pool 2

### Amplification of Plate and Assembly Subpools

As with the OLS Pool 1, oligos were synthesized, processed from the chip, lyophilized, and then reconstituted in 500 μL TE buffer. Plate subpools were made by amplifying 1 μL of OLS Pool 2 oligos in 50 μL reactions with the Phu4 PCR protocol using the forward and reverse plate-specific primers (skpp1 F and skpp1R). Fluorescent protein assembly subpools pools were amplified from the plate pool by including 20 mL of the plate subpool in 100 μL reactions that used the Phu4 protocol (except that the number of cycles was increased to 30) with the correct forward and reverse assembly-specific primers (skpp201F-skpp204F and skpp201R-skpp204R). The products were cleaned up using a QIAquick PCR Purification Kit, with the elution step conducted using 0.25×EB buffer diluted in water. The resulting DNA concentration of the assemblies was approximately 90 ng/4.

### Assembly

2 μL of each fluorescent protein assembly subpool were pre-assembled in 20 μL reactions following the KODpre protocol. 5 μL of each pre-assembly reaction was then assembled in 50 μL reactions following the KOD1 protocol and using the appropriate forward and reverse construction primers (skpp101F-skpp142F and skpp101R-skpp142R). The products were cleaned up using a MinElute PCR Purification Kit.

### Cloning

180 ng of mTFP1 assembly, 1.6 μg of mCitrine assembly, or 190 ng of mApple assembly were digested with HindIII and KpnI in 50 μL reactions identical to the one described for the cloning of the OLS Pool 1 constructs (except that the length of digest was 2 hours rather than 3 hours). The digested products were cleaned up using a MinElute PCR Purification Kit. The PCR-amplified KpnI/HindIII-digested 40 ng/μL GFPmut3 stock prepared during the first OLS Pool 1 assembly experiment was used as a positive control, and the 180 ng/μL stock of KpnI/HindIII-digested pZE21 prepared during the same earlier experiment was used as the cloning vector. Electrocompetent E. coli cells were prepared by concentrating a 2 L culture of NEB 5-alpha cells into 50 mL of water.

40 ng of pZE21 and either 60 ng of mTFP-BtsI-20 assembly, 90 ng of mCitrine-BtsI-20 assembly, 30 ng of mApple-BtsI-20, or 180 ng of control GFP were diluted in 8.5 μL water. The DNA was then ligated in a 10 μL T4 ligase reaction the products of which were electroporated into NEB 5-alpha cells following the protocol described in the cloning of the first OLS Chip 1 constructs. After an outgrowth of 37° C. for 70 minutes, 100 μL of the culture was diluted into 900 μL of LB with 50 μL/mL kanamycin, and another fraction was plated onto 50 μg/mL kanamycin LB agar plates. Both the plated cells and the cells in liquid culture were grown overnight at 37° C.

### Flow Cytometry

For each overnight culture, 20 μL was diluted into 2 mL 50 μg/μL kanamycin LB and grown at 37° C. for 2-3 hours. The fluorescent cell fraction was then quantified using a BD LSRFortessa flow cytometer.

### Optimizing ErrASE

### Error Correction

Error correction using ErrASE was performed using the manufacturer's instructions.

In brief, 2.4 μg of each fluorescent protein assembly (described above) were added to separate 60 μL reactions consisting of KOD polymerase buffer with 200 μM NTPs (EMD Chemicals) and 1.46 μM MgSO₄. Each reaction was heated to 98° C. for 1 minute, cooled to 0° C. for 5 minutes, kept at 37° C. for 5 minutes, and subsequently stored and handled at 4° C. 10 μL of the reaction was then added to each of the six ErrASE reactions of decreasing stringency, and the mix was incubated at 25° C. for 1-2 hours. The ErrASE reactions were then re-amplified by adding 2 μL to a 50 μL amplification reaction identical to KOD PCR used to assemble the fluorescent proteins.

### Cloning

Following error correction the amplifications that produced a band the size of a full-length assembly were cleaned up using a QIAquick PCR Purification Kit, with the DNA eluted into 30 μL of EB buffer. The error-corrected products were then digested with HindIII and KpnI in 50 μL reactions identical to the one described for the cloning of the OLS Pool 1 constructs. The digest was done at 37° C. for 3 hours while shaking at 800 rpm in a Thermomixer R. The digested products were cleaned up using a MinElute PCR Purification Kit. The PCR-amplified KpnI/HindIII-digested 40 ng/μL GFPmut3 stock prepared during the first OLS 1 assembly experiment was used as a positive control, and the 180 ng/μL stock of KpnI/HindIII-digested pZE21 prepared during the same earlier experiment was used as the cloning vector. Electrocompetent E. coli cells were prepared by concentrating a 2 L culture of NEB 5-alpha cells into 50 mL of water.

40 ng of pZE21 and 100-180 ng/μL of the inserts were ligated in a 10 μL T4 ligase reaction the products of which were electroporated into NEB 5-alpha cells following the protocol described in the cloning of the first OLS Chip 1 constructs. After electroporation the cells were outgrown in 1 mL of non-selective LB for 37° C. for 70 min, of which 100 μL was diluted into 900 μL of 50 ng/mL kanamycin LB and grown overnight at 37° C.

### Flow Cytometry

For each overnight culture, 20 μL was diluted into 2 mL 50 ng/mL kanamycin LB and grown at 37° C. for 2-3 hours. The fluorescent cell fraction was then quantified using a BD LSRFortessa flow cytometer using both a 488 nm blue laser with a FITC detector (530 nm filter with 30 nm bandpass) and a 561 nm yellow laser with a Texas Red detector (610 nm filter with a 20 nm bandpass).

Antibodies from the Second OLS Chip—First Set of Assemblies

### Amplification and Processing of Antibody Assembly Pools

Plate-specific assembly pools were amplified from the full set of 12,998 OLS 2 oligos in 50 μL Phu4 reactions with 1 μL OLS and using the plate-specific amplification primers skpp2F and skpp2R. To make antibody assembly subpools, 20 ng of the plate subpool was amplified in 100 μL reactions following the Phu5 protocol and using the appropriate forward and reverse amplification primers (skpp301F-skpp342F and skpp301R-skpp342R). The reaction was cleaned up using a QIAquick PCR Purification Kit, with each 100 μL reaction concentrated into 30 EB buffer. 30 μL of the amplified antibody assembly subpools were digested with BtsI in 40 μL reactions with 1× NEBuffer 4 (50 mM potassium acetate, 20 mM Tris acetate, 10 mM magnesium acetate, 1 mM DTT, pH 7.9; New England Biolabs), 125 ng/μL bovine serum albumin (New England Biolabs), and 0.5 units/4 BtsI (New England Biolabs). The reaction was cleaned up using a MinElute PCR Purification Kit.

### Assembly Optimization

125 ng of each antibody assembly subpool were pre-assembled in separate 20 μL reactions following the KODpre protocol. The assembly protocols have been named to facilitate cross-referencing with FIG. 10.

KOD-low: For each antibody, 100 nL of the pre-assembly reaction that has undergone the 15 thermal cycles but on which the final 72° C. extension had not been performed was amplified in a 50 μL KOD1 reaction using the appropriate construction primers (skpp101F-skpp142F and skpp101R-skpp142R).

KOD-high: For each antibody, 2 μL the full pre-assembly reaction was amplified in a 50 μL KOD1 reaction using the appropriate construction primers (skpp101F-skpp142F and skpp 101R-skpp142R).

KODXL65 and KODXL60: For each antibody, 100 nL the assembly reaction was amplified in 20 μL KODXL reactions using the appropriate forward and reverse construction primers. KODXL65 followed to the KODXL protocol exactly (with an annealing temperature of 65° C.), while KODXL60 used a 60° C. annealing temperature instead.

Phusion72, Phusion67, and Phusion62: For each antibody, 100 nL the assembly reaction was amplified in 20 μL Phu6 reactions with the appropriate forward and reverse construction primers. Phusion62 followed the Phu6 protocol exactly (using an annealing temperature of 62° C.), while Phusion72 and Phusion67 used annealing temperatures of 72° C. and 67° C., respectively.

Phusion67B, and Phusion62B: For each antibody, 100 nL the assembly reaction was amplified in 20 μL Phu6B reactions with the appropriate forward and reverse construction primers. Phusion62B followed the Phu6B protocol exactly (with the annealing temperature of 62° C.), while Phusion67B used an annealing temperature of 67° C.

### Amplification and Error Correction of a Subset of Antibodies

Based on the quality of the assemblies from the amplification optimization experiments, the following eight antibodies were chosen for cloning and characterization: efungumab, ibalizumab, panobacumab, ustekinumab, afutuzumab, oportuzumab, robatumumab, and vedolizumab. 10 mL of each pre-assembly was assembled in two 50 μL reactions following the Phu6B protocol using the appropriate forward and reverse primers. The reactions were cleaned up using a QIAquick PCR Purification Kit.

Error correction using ErrASE was performed as follows. 2 μL of each of the eight antibodies chosen were run a 2% E-Gel EX (Invitrogen) and reamplified by gel-stab PCR. Specifically, a 10 μL pipette tip was used to puncture the gel at the location of the desired product. The stab was mixed up and down in 10 μL of water, and the water was heated to 65° C. for 2 minutes. 2.5 μL of the gel-isolated product diluted in water was then amplified in a 50 μL Phu6B reaction. The following amount of the 8 antibody products were added to separate reactions consisting of KOD polymerase buffer (EMD chemicals, Gibbstown, N.J.) containing 200 μM NTPs (EMD chemicals, Gibbstown, N.J.) and 1.46 μM MgSO4: 920 ng of efungumab, 630 ng of ibalizumab, 190 ng of panobacumab, 910 ng of ustekinumab, 210 ng of afutuzumab, 360 ng of oportuzumab, 420 ng of robatumumab, and 910 ng of vedolizumab. Each reaction was heated to 98° C. for 1 minute, cooled to 0° C. for 5 minutes, kept at 37° C. for 5 minute, and subsequently stored and handled at 4° C. 10 μL of the reaction was added to each of the six ErrASE reactions, and the mix was incubated at 25° C. for 1 hour. The ErrASE reactions were then re-amplified by adding 2.5 μL of each ErrASE reaction to a 50 μL Phu7B reaction which used the appropriate construction primers.

### Cloning

The ErrASE-treated antibody assemblies were cleaned up using a QIAquick PCR Cleanup Kit, with the DNA eluted into 30 μL EB buffer. The 30 μL of DNA was then digested in a 100 μL reaction in FastDigest Buffer (Fermentas, Burligton, ON, Canada) that contained 4 μL of FastDigest ApaI (Fermentas) and 6 μL of FastDigest SfI (Fermentas). The reaction was kept first at 37° C. for 30 minutes, and then at 50° C. for 1 hour. The reactions were shaken at 800 rpm using a Thermomixer R during both thermal steps. 50 μg of the expression plasmid pSecTag2A (Invitrogen) was digested in a 100 μL of ApaI/SfiI digest similar to the one used to digest the antibody assemblies. Both the digested constructs and the digested plasmid were gel-isolated from a 2% agarose gel using a MinElute Gel Extraction Kit.

140-200 ng of one of the eight digested constructs and 90 ng of the digested plasmid were ligated in a 10 μL T4 ligase reaction the products of which were electroporated into NEB 5-alpha cells following the protocol described in the cloning of the first OLS Chip 1 constructs (with the following change: the 65° C. heat inactivation of the ligation was performed for only 10 minutes). The electroporated cells were suspended in 1 mL 2×YT medium, incubated at 37° C. for 45 min, and grown overnight on 50 μg/mL carbenicillin LB agar plates.

### Sequencing

After a night of growth, the plates with the cloned products were sent to GENEWIZ (South Plainfield, N.J.) for dideoxy sequencing. The following primers were used: forward: CMV-fwd (5′ CGCAAATGGGCGGTAGGCGTG) (SEQ ID NO:914); reverse: BGHR (5′ TAGAAGGCACAGTCGAGG) (SEQ ID NO:915). The trace files were analyzed using Lasergene 818. Deletions of more than two consecutive bases were counted as single errors. Clones that had errors in greater than 50% of the sequence were counted as misassemblies. Clones that did not have full sequence coverage between the two reads or that had traces that indicated that multiple clones were sequenced in the same reaction were counted as bad reads.

Antibodies from the Second OLS Chip—Second Set of Assemblies

### Amplification and Processing of Antibody Assembly Pools

Plate-specific assembly pools were amplified from the full set of 12,998 OLS 2 oligos in 50 μL Phu4 reactions with 1 μL OLS and using the plate-specific amplification primers skpp2F and skpp2R. To make antibody assembly subpools, 20 nL of the plate subpool was amplified in 100 μL reactions following the Phu5 protocol and using the appropriate forward and reverse amplification primers (skpp301F-skpp342F and skpp301R-skpp342R). The reaction was cleaned up using a QJAquick PCR Purification Kit, with four reactions concentrated into 120 μL EB buffer.

119 μL (2.2-15.9 μg) of the antibody assembly subpools were digested with BtsI in 129 μL reactions with 0.3× NEBuffer 4, 39 ng/μL bovine serum albumin (New England Biolabs), and 0.12 units/μL BtsI (New England Biolabs). The digest was performed at 55° C. at 2 hours while shaking at 1,000 rpm in the Thermomixer R. Each reaction was cleaned up using a MinElute PCR Purification Kit, with an elution into 15 of μL EB buffer. The resulting DNA concentrations ranged between 65 and 465 ng/μL, and were subsequently normalized to 50 ng/μL by adding EB buffer.

### Assembly

400 ng of each antibody assembly subpool were pre-assembled in separate 20 μL reactions following the KOD pre-protocol (except without the final 5 minutes at 72° C. extension). 10 nL of each pre-assembly reaction was then assembled into full-length genes using 50 μL Phu7B reactions (except that the 72° C. step during cycling was extended to 20 seconds) with the appropriate construction primers. Each pre-assembly was assembled in four separate reactions which were subsequently pooled. 185 μL of the assemblies were cleaned up using the QIAquick 96 PCR Purification Kit (QIAGEN), eluting into 60 μL EB with a final yield of 10-80 ng/μL.

The two antibodies that did not result in strong bands of the correct size (alacizumab and otelixizumab) were gel-stab isolated and re-amplified as follows. 20 μL of each antibody was run on a 2% E-Gel EX. A 10 μL pipette tip was used to puncture the gel at the location of the desired product. The stab was mixed up and down in 10 μL of water, and the water was heated to 60° C. for 5-20 minutes while being shaken at 750-800 rpm by the Thermomixer R. 1 μL the water containing the gel-isolated assemblies was then amplified in a 20 μL Phu8B reaction.

### Error Correction

Error correction using ErrASE was performed as described previously. In brief, 400 ng of abagovomab, 520 ng of alemtuzumab, 670 ng of cetuximab, 610 ng of efungumab, 310 ng of pertuzumab, 640 ng of ranibizumab, 240 ng of tadocizumab, or 660 ng of trastuzumab assembly were added to separate reactions consisting of HF Phusion buffer with 200 μM of each dNTP (Enzymatics) and either 1.5 M or no betaine (USB) (except for trastuzumab, which was error corrected only in a reaction lacking betaine). Each reaction was heated to 98° C. for 1 minute, cooled to 0° C. for 5 minutes, kept at 37° C. for 5 minutes, and subsequently stored and handled at 4° C. 10 μL of the reaction was added to each of the six ErrASE reactions, and the mix was incubated at 25° C. for 1 hour. The ErrASE reactions were then re-amplified by adding 2 μL of each ErrASE reaction to a 50 μL Phu8B reaction that used the appropriate construction primers.

### Cloning

10 μg of pSecTag2A was digested in a 50 μL reaction in NEBuffer 4 with 100 ng/μL bovine serum albumin (NEB) and 2 units/μL ApaI (NEB). The digest was done for 1 hour at 25° C. with shaking at 800 rpm by the Thermomixer R. At the conclusion, 2.5 μL (50 units) of SflI (NEB) were added, and another digest was performed for 1 hour at 50° C. with shaking at 800 rpm. 0.4 μL (2 units) of Antarctic phosphatase (NEB) and 5 μL of Antarctic phosphatase buffer were then added, and the reaction was allowed to proceed at 37° C. for 1 hour with 800 rpm shaking. The enzymes were inactivated by heating to 70° C. for 5 minutes while shaking at 800 rpm.

The best ErrASE reactions were cleaned up using a QIAquick PCR Cleanup Kit, with the DNA eluted into 30 μL EB buffer. 29 μL (0.15-1.95 μL of each assembly were digested in 50 μL reactions with NEBuffer, 100 ng/μL bovine serum albumin (NEB), and 0.8 units/μL ApaI (NEB). After 1 hour at 25° C. with 800 rpm shaking, 0.5 μL (10 units) of SfiI were added and the reaction was completed with 1 hour at 50° C. with 800 rpm shaking.

Both the digested constructs and the digested plasmid were gel-isolated from a 2% agarose gel using a MinElute Gel Extraction Kit. 60-175 ng of each of the digested constructs and 25 ng of the digested plasmid were ligated in a 10 μL T4 ligase reaction the products of which were electroporated into NEB 5-alpha cells following the protocol described in the cloning of the first OLS Chip 1 constructs. The electroporated cells were suspended in 1 mL EB medium, incubated at 37° C. for 70 minutes, and grown overnight on 50 μg/mL carbenicillin LB agar plates. Clones were picked, sequenced and analyzed as described in the cloning of the first set of antibody assemblies from the second OLS chip.

TABLE 14

| | | | | | Other | |
|---|---|---|---|---|---|---|
| Name | Buffer | Polymerase | Primers | dNTPs | Components | Thermocycling |
| KAPA- | 1x KAPA | Included | 500 nM | Included in | | 95° C.-1 min |
| prep | SYBR FAST | in Master | each | Master Mix | | cycle till plateau: |
| | qPCR | Mix | | | | (95° C.-10 s |
| | Master Mix | | | | | 62° C.-30 s) |
| | (Kapa | | | | | using BioRad CFX96 (Bio- |
| | Biosystems, | | | | | Rad Laboratories, |
| | Woburn | | | | | Hercules CA) |
| | MA) | | | | | |
| TaqPrep | 1x Taq | 0.02 U/μL | 500 nM | 200 μM each | | 94° C.-3 min |
| | Polymerase | Taq | each | (Enzymatics) | | 35 cycles of: |
| | (Enzymatics, | (Enzymatics) | | | | (94° C.-10 s |
| | Beverly | | | | | 62° C.-60 s) |
| | MA) | | | | | 72-5 min |
| | | | | | | using DNA Engine Tetrad |
| | | | | | | 2 (Bio-Rad) |
| Phu1 | 1x Phusion | 0.02 U/μL | 500 nM | 200 μM each | | 98° C.-30 s |
| | HF | Phusion | each | (Enzymatics) | | 30 cycles of: |
| | (Finnzymes, | (Finnzymes) | | | | (98° C.-5 s |
| | Woburn, | | | | | 51° C.-10 s |
| | MA) | | | | | 72° C.-30 s) |
| | | | | | | 72-10 min |
| | | | | | | using Tetrad 2 |
| Phu2 | 1x Phusion | 0.02 U/μL | 500 nM | 200 μM each | | 98° C.-30 s |
| | HF | Phusion | each | (Enzymatics) | | 30 cycles of: |
| | | | | | | (98° C.-5 s |
| | | | | | | 72° C.-30 s) |
| | | | | | | 72-10 min |
| | | | | | | using Tetrad 2 |
| Phu3 | 1x Phusion | 0.02 U/μL | 250 nM | 200 μM each | | 98° C.-30 s |
| | HF | Phusion | each | (Enzymatics) | | 30 cycles of: |
| | | | | | | (98° C.-5 s |
| | | | | | | 72° C.-30 s) |
| | | | | | | 72-5 min |
| | | | | | | using Tetrad 2 |
| Phu4 | 1x Phusion | 0.02 U/μL | 500 nM | 200 μM each | | 98° C.-30 s |
| | HF | Phusion | each | (Enzymatics) | | 25 cycles of: |
| | | | | | | (98° C.-5 s |
| | | | | | | 65° C.-10 s |
| | | | | | | 72° C.-10 s) |
| | | | | | | 72-5 min |
| | | | | | | using Tetrad 2 |
| Phu5 | 1x Phusion | 0.02 U/μL | 1 μM | 200 μM | | 98° C.-30 s |
| | HF | Phusion | each | (Enzymatics) | | 30 cycles of: |
| | | | | | | (98° C.-5 s |
| | | | | | | 65° C.-10 s |
| | | | | | | 72° C.-10 s) |
| | | | | | | 72-5 min |
| | | | | | | using Tetrad 2 |
| Phu6 | 1x Phusion | 0.02 U/μL | 500 nM | 200 μM each | | 98° C.-30 s |
| | HF | Phusion | each | (Enzymatics) | | 25 cycles of: |
| | | | | | | (98° C.-5 s |
| | | | | | | 62° C.-5 s |
| | | | | | | 72° C.-10 s) |
| | | | | | | 72-10 min |
| | | | | | | using Tetrad 2 |
| Phu6B | 1x Phusion | 0.02 U/μL | 500 nM | 200 μM each | 2M betaine | 98° C.-30 s |
| | HF | Phusion | each | (Enzymatics) | (USB, | 25 cycles of: |
| | | | | | Cleveland OH) | (98° C.-5 s |
| | | | | | | 62° C.-5 s |
| | | | | | | 72° C.-10 s) |
| | | | | | | 72-10 min |
| | | | | | | using Tetrad 2 |
| Phu7B | 1x Phusion | 0.02 U/μL | 500 nM | 200 μM each | 2M betaine | 98° C.-30 s |
| | HF | Phusion | each | (Enzymatics) | (USB) | 25 cycles of: |
| | | | | | | (98° C.-5 s |
| | | | | | | 62° C.-10 s |
| | | | | | | 72° C.-15 s) |
| | | | | | | 72-5 min |
| | | | | | | using Tetrad 2 |
| Phu8B | 1x Phusion | 0.02 U/μL | 500 nM | 200 μM each | 2M betaine | 98° C.-30 s |
| | HF | Phusion | each | (Enzymatics) | (USB) | 30 cycles of: |
| | | | | | | (98° C.-5 s |
| | | | | | | 62° C.-10 s |
| | | | | | | 72° C.-20 s) |
| | | | | | | 72-5 min |
| | | | | | | using Tetrad 2 |
| KODpre | 1x KOD | 0.02 U/μL | | 200 μM each | 1.5 mM | 95° C.-2 min |
| | Polymerase | KOD | | (EMD | MgSO₄ (EMD | 15 cycles of: |
| | (EMD | (EMD | | Chemicals) | Chemicals) | (95° C.-20 s |
| | Chemicals, | Chemicals) | | | | 70° C.-1 s |
| | Gibbstown | | | | | 0.5° C./s ramp to 50° C. |
| | NJ) | | | | | 50° C.-30 s |
| | | | | | | 72° C.-20 s) |
| | | | | | | 72-5 min |
| | | | | | | using Tetrad 2 |
| KOD1 | 1x KOD | 0.02 U/μL | 200 nM | 200 μM each | 1.5 mM | 95° C.-2 min |
| | Polymerase | KOD | each | (EMD | MgSO₄ (EMD | 25 cycles of: |
| | | | | Chemicals) | Chemicals) | (95° C.-20 s |
| | | | | | | 60° C.-30 s |
| | | | | | | 72° C.-20 s) |
| | | | | | | 72-5 min |
| | | | | | | using Tetrad 2 |
| KODXL | KOD XL | 0.05 U/μL | 400 nM | 200 μM each | | 94° C.-30 s |
| | Polymerase | KOD XL | | (EMD | | 25 cycles of: |
| | (EMB | (EMB | | Chemicals) | | (94° C.-20 s |
| | Chemicals) | Chemicals) | | | | 65° C.-5 s |
| | | | | | | 74° C.-30 s) |
| | | | | | | 74-10 min |
| | | | | | | using Tetrad 2 |

Table 14 sets forth PCR methods described herein.

What is claimed is:

1. A microarray comprising at least 5,000 different oligonucleotide sequences attached thereto,
wherein each oligonucleotide sequence is a member of one of a plurality of oligonucleotide sets, and each oligonucleotide set is specific for a nucleic acid sequence of interest,
wherein each oligonucleotide sequence that is a member of a particular oligonucleotide set includes a pair of orthogonal primer binding sites having a sequence that is unique to said oligonucleotide set, and
wherein the nucleic acid sequence of interest is at least 500 nucleotides in length.

2. The microarray of claim 1, wherein at least 50 oligonucleotide sets are provided, and wherein each set is specific for a unique nucleic acid sequence of interest.

3. The microarray of claim 1, wherein at least 100 oligonucleotide sets are provided, and wherein each set is specific for a unique nucleic acid sequence of interest.

4. The microarray of claim 1, wherein the oligonucleotide sequence of interest is at least 1,000 nucleotides in length.

5. The microarray of claim 1, wherein the oligonucleotide sequence of interest is at least 2,500 nucleotides in length.

6. The microarray of claim 1, wherein the oligonucleotide sequence of interest is at least 5,000 nucleotides in length.

7. The microarray of claim 1, wherein the nucleic acid sequence of interest is a DNA sequence.

8. The microarray of claim 7, wherein the DNA sequence is selected from the group consisting of a regulatory element, a gene, a pathway and a genome.

9. The microarray of claim 1, comprising at least 10,000 different oligonucleotide sequences attached thereto.

10. The microarray of claim 1, wherein an oligonucleotide set is specific for a single nucleic acid sequence of interest.

11. A microarray comprising at least 10,000 different oligonucleotide sequences attached thereto,
wherein each oligonucleotide sequence is a member of one of at least 50 oligonucleotide sets, and each oligonucleotide set is specific for a nucleic acid sequence of interest,
wherein each oligonucleotide sequence that is a member of a particular oligonucleotide set includes a pair of orthogonal primer binding sites having a sequence that is unique to said oligonucleotide set, and
wherein each nucleic acid sequence of interest is at least 2,500 nucleotides in length.

12. A method of synthesizing a nucleic acid sequence of interest comprising the steps of:
providing at least 5,000 different oligonucleotide sequences, wherein each oligonucleotide sequence is a member of one of a plurality of oligonucleotide sets, and each oligonucleotide set is specific for a nucleic acid sequences of interest, and wherein each oligonucleotide sequence includes a pair of orthogonal primer binding sites having a sequence that is unique to a single oligonucleotide set;
amplifying an oligonucleotide set using orthogonal primers that hybridize to the orthogonal primer binding sites unique to the set;
removing the orthogonal primer binding sites from the amplified oligonucleotide set; and
assembling the amplified oligonucleotide set into a nucleic acid sequence of interest that is at least 500 nucleotides in length.

13. The method of claim 12, wherein the nucleic acid sequence of interest is at least 1,000 nucleotides in length.

14. The method of claim 12, wherein the nucleic acid sequence of interest is at least 2,500 nucleotides in length.

15. The method of claim 12, wherein the nucleic acid sequence of interest is at least 5,000 nucleotides in length.

16. The method of claim 12, wherein the nucleic acid sequence of interest is a DNA sequence.

17. The method of claim 16, wherein the DNA sequence is selected from the group consisting of a regulatory element, a gene, a pathway and a genome.

18. The method of claim 12, wherein 50 oligonucleotide sets are provided, and wherein each set is specific for a unique nucleic acid sequence of interest.

19. The method of claim 12, wherein 100 oligonucleotide sets are provided, and wherein each set is specific for a unique nucleic acid sequence of interest.

20. The method of claim 12, wherein 500 oligonucleotide sets are provided, and wherein each set is specific for a unique nucleic acid sequence of interest.

21. The method of claim 12, wherein 750 oligonucleotide sets are provided, and wherein each set is specific for a unique nucleic acid sequence of interest.

22. The method of claim 12, wherein 1,000 oligonucleotide sets are provided, and wherein each set is specific for a unique nucleic acid sequence of interest.

23. The method of claim 12, wherein the 5,000 different oligonucleotide sequences are provided on a microarray.

24. The method of claim 23, wherein the 5,000 different oligonucleotide sequences are removed from the microarray prior to the step of amplifying.


SEQUENCE LISTING (889 sequences)

SEQ ID NO: 1 - protein, 17 aa, Artificial
  Synthetic construct designed to act as linker
GGSGGSGGASGAGSGGG

SEQ ID NO: 2 - protein, 17 aa, Artificial
  Synthetic construct designed to act as linker
GGSAGSGSSGGASGSGG

SEQ ID NO: 3 - protein, 16 aa, Artificial
  Synthetic construct designed to act as linker
GAGSGAGSGSSGAGSG

SEQ ID NO: 4 - protein, 17 aa, Artificial
  Synthetic construct designed to act as linker
GGSGGSGGASGAGSGGG

SEQ ID NO: 5 - protein, 17 aa, Artificial
  Synthetic construct designed to act as linker
GGSGGSGGASGAGSGGG

SEQ ID NO: 6 - protein, 17 aa, Artificial
  Synthetic construct designed to act as linker
GGSGGSGGASGAGSGGG

SEQ ID NO: 7 - protein, 17 aa, Artificial
  Synthetic construct designed to act as linker
GGSGGSGGASGAGSGGG

SEQ ID NO: 8 - protein, 17 aa, Artificial
  Synthetic construct designed to act as linker
GGSGGSGGASGAGSGGG

SEQ ID NO: 9 - protein, 17 aa, Artificial
  Synthetic construct designed to act as linker
GGSGGSGGASGAGSGGG

SEQ ID NO: 10 - protein, 17 aa, Artificial
  Synthetic construct designed to act as linker
GGSGGSGGASGAGSGGG

SEQ ID NO: 11 - protein, 17 aa, Artificial
  Synthetic construct designed to act as linker
GGSGGSGGASGAGSGGG

SEQ ID NO: 12 - protein, 17 aa, Artificial
  Synthetic construct designed to act as linker
GGSGGSGGASGAGSGGG

SEQ ID NO: 13 - protein, 17 aa, Artificial
  Synthetic construct designed to act as linker
GGSGGSGGASGAGSGGG

SEQ ID NO: 14 - protein, 17 aa, Artificial
  Synthetic construct designed to act as linker
GGSGGSGGASGAGSGGG

SEQ ID NO: 15 - protein, 17 aa, Artificial
  Synthetic construct designed to act as linker
GGSGGSGGASGAGSGGG

SEQ ID NO: 16 - protein, 17 aa, Artificial
  Synthetic construct designed to act as linker
GGSGGSGGASGAGSGGG

SEQ ID NO: 17 - protein, 17 aa, Artificial
  Synthetic construct designed to act as linker
GGSGGSGGASGAGSGGG

SEQ ID NO: 18 - protein, 17 aa, Artificial
  Synthetic construct designed to act as linker
GGSGGSGGASGAGSGGG

SEQ ID NO: 19 - protein, 17 aa, Artificial
  Synthetic construct designed to act as linker
GGSAGSGSSGGASGSGG

SEQ ID NO: 20 - protein, 17 aa, Artificial
  Synthetic construct designed to act as linker
GGSAGSGSSGGASGSGG

SEQ ID NO: 21 - protein, 17 aa, Artificial
  Synthetic construct designed to act as linker
GGSAGSGSSGGASGSGG

SEQ ID NO: 22 - protein, 17 aa, Artificial
  Synthetic construct designed to act as linker
GGSAGSGSSGGASGSGG

SEQ ID NO: 23 - protein, 17 aa, Artificial
  Synthetic construct designed to act as linker
GGSAGSGSSGGASGSGG

SEQ ID NO: 24 - protein, 17 aa, Artificial
  Synthetic construct designed to act as linker
GGSAGSGSSGGASGSGG

SEQ ID NO: 25 - protein, 17 aa, Artificial
  Synthetic construct designed to act as linker
GGSAGSGSSGGASGSGG

SEQ ID NO: 26 - protein, 17 aa, Artificial
  Synthetic construct designed to act as linker
GGSAGSGSSGGASGSGG

SEQ ID NO: 27 - protein, 17 aa, Artificial
  Synthetic construct designed to act as linker
GGSAGSGSSGGASGSGG

SEQ ID NO: 28 - protein, 17 aa, Artificial
  Synthetic construct designed to act as linker
GGSAGSGSSGGASGSGG

SEQ ID NO: 29 - protein, 17 aa, Artificial
  Synthetic construct designed to act as linker
GGSAGSGSSGGASGSGG

SEQ ID NO: 30 - protein, 17 aa, Artificial
  Synthetic construct designed to act as linker
GGSAGSGSSGGASGSGG

SEQ ID NO: 31 - protein, 17 aa, Artificial
  Synthetic construct designed to act as linker
GGSAGSGSSGGASGSGG

SEQ ID NO: 32 - protein, 17 aa, Artificial
  Synthetic construct designed to act as linker
GGSAGSGSSGGASGSGG

SEQ ID NO: 33 - protein, 16 aa, Artificial
  Synthetic construct designed to act as linker
GAGSGAGSGSSGAGSG

SEQ ID NO: 34 - protein, 16 aa, Artificial
  Synthetic construct designed to act as linker
GAGSGAGSGSSGAGSG

SEQ ID NO: 35 - protein, 16 aa, Artificial
  Synthetic construct designed to act as linker
GAGSGAGSGSSGAGSG

SEQ ID NO: 36 - protein, 16 aa, Artificial
  Synthetic construct designed to act as linker
GAGSGAGSGSSGAGSG

SEQ ID NO: 37 - protein, 16 aa, Artificial
  Synthetic construct designed to act as linker
GAGSGAGSGSSGAGSG

SEQ ID NO: 38 - protein, 16 aa, Artificial
  Synthetic construct designed to act as linker
GAGSGAGSGSSGAGSG

SEQ ID NO: 39 - protein, 16 aa, Artificial
  Synthetic construct designed to act as linker
GAGSGAGSGSSGAGSG

SEQ ID NO: 40 - protein, 16 aa, Artificial
  Synthetic construct designed to act as linker
GAGSGAGSGSSGAGSG

SEQ ID NO: 41 - protein, 16 aa, Artificial
  Synthetic construct designed to act as linker
GAGSGAGSGSSGAGSG

SEQ ID NO: 42 - protein, 16 aa, Artificial
  Synthetic construct designed to act as linker
GAGSGAGSGSSGAGSG

SEQ ID NO: 43 - protein, 16 aa, Artificial
  Synthetic construct designed to act as linker
GAGSGAGSGSSGAGSG

SEQ ID NO: 44 - protein, 16 aa, Artificial
  Synthetic construct designed to act as linker
GAGSGAGSGSSGAGSG

SEQ ID NO: 45 - protein, 16 aa, Artificial
  Synthetic construct designed to act as linker
GAGSGAGSGSSGAGSG

SEQ ID NO: 46 - DNA, 20 bp, Artificial
  Primer
aacacgtccgtcctagaact

SEQ ID NO: 47 - DNA, 20 bp, Artificial
  Primer
agtgttgagcgtaaccaagt

SEQ ID NO: 48 - DNA, 20 bp, Artificial
  Primer
aagcaagattctcgtcggat

SEQ ID NO: 49 - DNA, 20 bp, Artificial
  Primer
tctaatctagcgcgacgtct

SEQ ID NO: 50 - DNA, 20 bp, Artificial
  Primer
gcaagcggtacactcagatc

SEQ ID NO: 51 - DNA, 20 bp, Artificial
  Primer
caggagttgtctaggcgatc

SEQ ID NO: 52 - DNA, 20 bp, Artificial
  Primer
tgtaaggcacatctcggatc

SEQ ID NO: 53 - DNA, 20 bp, Artificial
  Primer
ccacaagaggcgctatgatc

SEQ ID NO: 54 - DNA, 122 bp, Artificial
  Primer
aacacgtccgtcctagaactgatagggtgactgctttcgcgtacaggtaccatgagtaaaggagaagaacttttcactggagttgtcccaattcttgttgaagatctgagtgtaccgcttgc

SEQ ID NO: 55 - DNA, 128 bp, Artificial
  Primer
aacacgtccgtcctagaactttagatggtgatgttaatgggcacaaattttctgtcagtggagagggtgaaggtgatgcaacatacggaaaacttacccttaaatttagatctgagtgtaccgcttgc

SEQ ID NO: 56 - DNA, 122 bp, Artificial
  Primer
aacacgtccgtcctagaacttttgcactactggaaaactacctgttccatggccaacacttgtcactactttcggttatggtgttcaatgctttgcgagatagatctgagtgtaccgcttgc

SEQ ID NO: 57 - DNA, 123 bp, Artificial
  Primer
aacacgtccgtcctagaactcccagatcatatgaaacagcatgactttttcaagagtgccatgcccgaaggttatgtacaggaaagaactatatttttcaaaggatctgagtgtaccgcttgc

SEQ ID NO: 58 - DNA, 126 bp, Artificial
  Primer
aacacgtccgtcctagaactatgacgggaactacaagacacgtgctgaagtcaagtttgaaggtgatacccttgttaatagaatcgagttaaaaggtattgattttgatctgagtgtaccgcttgc

SEQ ID NO: 59 - DNA, 130 bp, Artificial
  Primer
aacacgtccgtcctagaactaaagaagatggaaacattcttggacacaaattggaatacaactataactcacacaatgtatacatcatggcagacaaacaaaagaatggagatctgagtgtaccgcttgc

SEQ ID NO: 60 - DNA, 130 bp, Artificial
  Primer
aacacgtccgtcctagaactatcaaagttaacttcaaaattagacacaacattgaagatggaagcgttcaactagcagaccattatcaacaaaatactccaattggcgatgatctgagtgtaccgcttgc

SEQ ID NO: 61 - DNA, 128 bp, Artificial
  Primer
aacacgtccgtcctagaactggccctgtccttttaccagacaaccattacctgtccacacaatctgccctttcgaaagatcccaacgaaaagagagaccacatggtccgatctgagtgtaccgcttgc

SEQ ID NO: 62 - DNA, 130 bp, Artificial
  Primer
aacacgtccgtcctagaactttcttgagtttgtaacagctgctgggattacacatggcatggatgaactatacaaataaaagcttacttcttctcggtcgcatgaggctggatctgagtgtaccgcttgc

SEQ ID NO: 63 - DNA, 124 bp, Artificial
  Primer
aacacgtccgtcctagaactctccactgacagaaaatttgtgcccattaacatcaccatctaattcaacaagaattgggacaactccagtgaaaagttcttctcgatctgagtgtaccgcttgc

SEQ ID NO: 64 - DNA, 126 bp, Artificial
  Primer
aacacgtccgtcctagaactaagtgttggccatggaacaggtagttttccagtagtgcaaataaatttaagggtaagttttccgtatgttgcatcaccttcaccctgatctgagtgtaccgcttgc

SEQ ID NO: 65 - DNA, 123 bp, Artificial
  Primer
aacacgtccgtcctagaactatggcactcttgaaaaagtcatgctgtttcatatgatctgggtatctcgcaaagcattgaacaccataaccgaaagtagtgacgatctgagtgtaccgcttgc

SEQ ID NO: 66 - DNA, 122 bp, Artificial
  Primer
aacacgtccgtcctagaactttcaaacttgacttcagcacgtgtcttgtagttcccgtcatctttgaaaaatatagttctttcctgtacataaccttcgggcgatctgagtgtaccgcttgc

SEQ ID NO: 67 - DNA, 130 bp, Artificial
  Primer
aacacgtccgtcctagaactatagttgtattccaatttgtgtccaagaatgtttccatcttctttaaaatcaataccttttaactcgattctattaacaagggtatcaccgatctgagtgtaccgcttgc

SEQ ID NO: 68 - DNA, 130 bp, Artificial
  Primer
aacacgtccgtcctagaactgcttccatcttcaatgttgtgtctaattttgaagttaactttgattccattcttttgtttgtctgccatgatgtatacattgtgtgagttgatctgagtgtaccgcttgc

SEQ ID NO: 69 - DNA, 130 bp, Artificial
  Primer
aacacgtccgtcctagaactagattgtgtggacaggtaatggttgtctggtaaaaggacagggccatcgccaattggagtattttgttgataatggtctgctagttgaacgatctgagtgtaccgcttgc

SEQ ID NO: 70 - DNA, 128 bp, Artificial
  Primer
aacacgtccgtcctagaactcatccatgccatgtgtaatcccagcagctgttacaaactcaagaaggaccatgtggtctctcttttcgttgggatctttcgaaagggcgatctgagtgtaccgcttgc

SEQ ID NO: 71 - DNA, 126 bp, Artificial
  Primer
aacacgtccgtcctagaactctttactcatggtacctgtacgcgaaagcagtcaccctatccagcctcatgcgaccgagaagaagtaagcttttatttgtatagttgatctgagtgtaccgcttgc

SEQ ID NO: 72 - DNA, 108 bp, Artificial
  Primer
agtgttgagcgtaaccaagtgatagggtgactgctttcgcgtacaggtaccatgagtaaaggagaagaacttttcactggagttgtccgatcgcctagacaactcctg

SEQ ID NO: 73 - DNA, 111 bp, Artificial
  Primer
agtgttgagcgtaaccaagtcaattcttgttgaattagatggtgatgttaatgggcacaaattttctgtcagtggagagggtgaaggtgatgatcgcctagacaactcctg

SEQ ID NO: 74 - DNA, 110 bp, Artificial
  Primer
agtgttgagcgtaaccaagtgcaacatacggaaaacttacccttaaatttatttgcactactggaaaactacctgttccatggccaacacgatcgcctagacaactcctg

SEQ ID NO: 75 - DNA, 108 bp, Artificial
  Primer
agtgttgagcgtaaccaagtttgtcactactttcggttatggtgttcaatgctttgcgagatacccagatcatatgaaacagcatgacgatcgcctagacaactcctg

SEQ ID NO: 76 - DNA, 108 bp, Artificial
  Primer
agtgttgagcgtaaccaagttttttcaagagtgccatgcccgaaggttatgtacaggaaagaactatatttttcaaagatgacgggaagatcgcctagacaactcctg

SEQ ID NO: 77 - DNA, 109 bp, Artificial
  Primer
agtgttgagcgtaaccaagtctacaagacacgtgctgaagtcaagtttgaaggtgatacccttgttaatagaatcgagttaaaaggtatgatcgcctagacaactcctg

SEQ ID NO: 78 - DNA, 117 bp, Artificial
  Primer
agtgttgagcgtaaccaagttgattttaaagaagatggaaacattcttggacacaaattggaatacaactataactcacacaatgtatacatcatgggatcgcctagacaactcctg

SEQ ID NO: 79 - DNA, 113 bp, Artificial
  Primer
agtgttgagcgtaaccaagtcagacaaacaaaagaatggaatcaaagttaacttcaaaattagacacaacattgaagatggaagcgttcaactgatcgcctagacaactcctg

SEQ ID NO: 80 - DNA, 110 bp, Artificial
  Primer
agtgttgagcgtaaccaagtagcagaccattatcaacaaaatactccaattggcgatggccctgtccttttaccagacaaccattacctggatcgcctagacaactcctg

SEQ ID NO: 81 - DNA, 111 bp, Artificial
  Primer
agtgttgagcgtaaccaagttccacacaatctgccctttcgaaagatcccaacgaaaagagagaccacatggtccttcttgagtttgtaacgatcgcctagacaactcctg

SEQ ID NO: 82 - DNA, 114 bp, Artificial
  Primer
agtgttgagcgtaaccaagtagctgctgggattacacatggcatggatgaactatacaaataaaagcttacttcttctcggtcgcatgaggctggatcgcctagacaactcctg

SEQ ID NO: 83 - DNA, 116 bp, Artificial
  Primer
agtgttgagcgtaaccaagttgtgcccattaacatcaccatctaattcaacaagaattgggacaactccagtgaaaagttcttctcctttactcatgatcgcctagacaactcctg

SEQ ID NO: 84 - DNA, 111 bp, Artificial
  Primer
agtgttgagcgtaaccaagtagtgcaaataaatttaagggtaagttttccgtatgttgcatcaccttcaccctctccactgacagaaaattgatcgcctagacaactcctg

SEQ ID NO: 85 - DNA, 106 bp, Artificial
  Primer
agtgttgagcgtaaccaagtaaagcattgaacaccataaccgaaagtagtgacaagtgttggccatggaacaggtagttttccagtgatcgcctagacaactcctg

SEQ ID NO: 86 - DNA, 104 bp, Artificial
  Primer
agtgttgagcgtaaccaagtcataaccttcgggcatggcactcttgaaaaagtcatgctgtttcatatgatctgggtatctcgcgatcgcctagacaactcctg

SEQ ID NO: 87 - DNA, 108 bp, Artificial
  Primer
agtgttgagcgtaaccaagtttcaaacttgacttcagcacgtgtcttgtagttcccgtcatctttgaaaaatatagttctttcctgtagatcgcctagacaactcctg

SEQ ID NO: 88 - DNA, 116 bp, Artificial
  Primer
agtgttgagcgtaaccaagtatttgtgtccaagaatgtttccatcttctttaaaatcaataccttttaactcgattctattaacaagggtatcaccgatcgcctagacaactcctg

SEQ ID NO: 89 - DNA, 118 bp, Artificial
  Primer
agtgttgagcgtaaccaagtttttgaagttaactttgattccattcttttgtttgtctgccatgatgtatacattgtgtgagttatagttgtattccagatcgcctagacaactcctg

SEQ ID NO: 90 - DNA, 111 bp, Artificial
  Primer
agtgttgagcgtaaccaagtatcgccaattggagtattttgttgataatggtctgctagttgaacgcttccatcttcaatgttgtgtctaagatcgcctagacaactcctg

SEQ ID NO: 91 - DNA, 105 bp, Artificial
  Primer
agtgttgagcgtaaccaagtttgggatctttcgaaagggcagattgtgtggacaggtaatggttgtctggtaaaaggacagggccgatcgcctagacaactcctg

SEQ ID NO: 92 - DNA, 115 bp, Artificial
  Primer
agtgttgagcgtaaccaagttatagttcatccatgccatgtgtaatcccagcagctgttacaaactcaagaaggaccatgtggtctctcttttcggatcgcctagacaactcctg

SEQ ID NO: 93 - DNA, 109 bp, Artificial
  Primer
agtgttgagcgtaaccaagtggtacctgtacgcgaaagcagtcaccctatccagcctcatgcgaccgagaagaagtaagcttttatttggatcgcctagacaactcctg

SEQ ID NO: 94 - DNA, 130 bp, Artificial
  Primer
aagcaagattctcgtcggatccggacgactttattacagcgaaggaaaggtatactgaaatttaaaaaacgtagttaaacgattgcgttcaaatatttaatccttccggcgatccgagatgtgccttaca

SEQ ID NO: 95 - DNA, 130 bp, Artificial
  Primer
aagcaagattctcgtcggatgggattgtacccaatccacgctcttttttatagagaagatgacgttaaattggccagatattgtcgatgataatttgcaggctgcggttggatccgagatgtgccttaca

SEQ ID NO: 96 - DNA, 130 bp, Artificial
  Primer
aagcaagattctcgtcggatctctggaggcaagcttagcgcctctgttttatttttccatcagatagcgcttaactgaacaaggcttgtgcatgagcaataccgtctctcgatccgagatgtgccttaca

SEQ ID NO: 97 - DNA, 130 bp, Artificial
  Primer
aagcaagattctcgtcggataatccgcaacaaatcccgccagaaatcgcggcgttaattaattaagtatcctatgcaaaaagttgtcctcgcaaccggcaatgtcggtaagatccgagatgtgccttaca

SEQ ID NO: 98 - DNA, 130 bp, Artificial
  Primer
aagcaagattctcgtcggatgtggagcgtttgttacagcagttacgcactggcgcgccggtttaacgcgtgagtcgataaagaggatgatttatgagcagaacgatttttgatccgagatgtgccttaca

SEQ ID NO: 99 - DNA, 130 bp, Artificial
  Primer
aagcaagattctcgtcggatgccaccatttgattcgctcggcggtgccgctggagatgaacctgagttaactggtattaaatctgcttttcatacaatcggtaacgcttggatccgagatgtgccttaca

SEQ ID NO: 100 - DNA, 130 bp, Artificial
  Primer
aagcaagattctcgtcggatactgagtcagccgagaagaatttccccgcttattcgcaccttccttaaatcaggtcatacgcttcgagatacttaacgccaaacaccagcgatccgagatgtgccttaca

SEQ ID NO: 101 - DNA, 130 bp, Artificial
  Primer
aagcaagattctcgtcggattggttgatgcagaaaaagcgattacggattttatgaccgcgcgtggttatcactaatcaaaaatggaaatgcccgatcgccaggaccggggatccgagatgtgccttaca

SEQ ID NO: 102 - DNA, 130 bp, Artificial
  Primer
aagcaagattctcgtcggatttctctgtctatgagagccgttaaaacgactctcatagattttattaatagcaaaatataaaccgtccccaaaaaagccaccaaccacaagatccgagatgtgccttaca

SEQ ID NO: 103 - DNA, 130 bp, Artificial
  Primer
aagcaagattctcgtcggatagggttaacaggctttccaaatggtgtccttaggtttcacgacgttaataaaccggaatcgccatcgctccatgtgctaaacagtatcgcgatccgagatgtgccttaca

SEQ ID NO: 104 - DNA, 130 bp, Artificial
  Primer
tctaatctagcgcgacgtctgcatcgtaaagaacattttgaggcatttcagtcagttgctcaatgtacctataaccagaccgttcagctggatattacggcctttttaaagatcatagcgcctcttgtgg

SEQ ID NO: 105 - DNA, 130 bp, Artificial
  Primer
tctaatctagcgcgacgtctcgcgattaaattccaacatggatgctgatttatatgggtataaatgggctcgcgataatgtcgggcaatcaggtgcgacaatctatcgctgatcatagcgcctcttgtgg

SEQ ID NO: 106 - DNA, 130 bp, Artificial
  Primer
tctaatctagcgcgacgtctccaaatgacatgttttctgctactgacaggtggggatagagcgcttaagactgaaacaccataccaacgccgcgttctgctggcggagtggatcatagcgcctcttgtgg

SEQ ID NO: 107 - DNA, 130 bp, Artificial
  Primer
tctaatctagcgcgacgtctggaaacagctatgaccatgattacggattcactggccgtcgtttgacaacgtcgtgactgggaaaaccctggcgttacccaacttaatcggatcatagcgcctcttgtgg

SEQ ID NO: 108 - DNA, 130 bp, Artificial
  Primer
tctaatctagcgcgacgtctagcctttctgggttcagttcgttgagccaggccgagaacctgatgcaagtttatcagcaagcacgccttagtaacccggaattgcgtaaggatcatagcgcctcttgtgg

SEQ ID NO: 109 - DNA, 35 bp, Artificial
  Primer
gatagggtgactgctttcgcgtacaggtaccatga

SEQ ID NO: 110 - DNA, 40 bp, Artificial
  Primer
gtaaaggagaagaacttttcactggagttgtcccaattct

SEQ ID NO: 111 - DNA, 42 bp, Artificial
  Primer
tgttgaattagatggtgatgttaatgggcacaaattttctgt

SEQ ID NO: 112 - DNA, 43 bp, Artificial
  Primer
aacttacccttaaatttatttgcactactggaaaactacctgt

SEQ ID NO: 113 - DNA, 37 bp, Artificial
  Primer
tccatggccaacacttgtcactactttcggttatggt

SEQ ID NO: 114 - DNA, 37 bp, Artificial; identical to SEQ ID NO: 113
  Primer

SEQ ID NO: 115 - DNA, 37 bp, Artificial
  Primer
catgactttttcaagagtgccatgcccgaaggttatg

SEQ ID NO: 116 - DNA, 42 bp, Artificial
  Primer
tacaggaaagaactatatttttcaaagatgacgggaactaca

SEQ ID NO: 117 - DNA, 37 bp, Artificial
  Primer
agacacgtgctgaagtcaagtttgaaggtgataccct

SEQ ID NO: 118 - DNA, 46 bp, Artificial
  Primer
tgttaatagaatcgagttaaaaggtattgattttaaagaagatgga

SEQ ID NO: 119 - DNA, 44 bp, Artificial
  Primer
aacattcttggacacaaattggaatacaactataactcacacaa

SEQ ID NO: 120 - DNA, 43 bp, Artificial
  Primer
tgtatacatcatggcagacaaacaaaagaatggaatcaaagtt

SEQ ID NO: 121 - DNA, 41 bp, Artificial
  Primer
aacttcaaaattagacacaacattgaagatggaagcgttca

SEQ ID NO: 122 - DNA, 40 bp, Artificial
  Primer
actagcagaccattatcaacaaaatactccaattggcgat

SEQ ID NO: 123 - DNA, 36 bp, Artificial
  Primer
ggccctgtccttttaccagacaaccattacctgtcc

SEQ ID NO: 124 - DNA, 37 bp, Artificial
  Primer
acacaatctgccctttcgaaagatcccaacgaaaaga

SEQ ID NO: 125 - DNA, 36 bp, Artificial
  Primer
gagaccacatggtccttcttgagtttgtaacagctg

SEQ ID NO: 126 - DNA, 41 bp, Artificial
  Primer
ctgggattacacatggcatggatgaactatacaaataaaag

SEQ ID NO: 127 - DNA, 35 bp, Artificial
  Primer
cttacttcttctcggtcgcatgaggctgatcagcg

SEQ ID NO: 128 - DNA, 39 bp, Artificial
  Primer
gtgaaaagttcttctcctttactcatggtacctgtacgc

SEQ ID NO: 129 - DNA, 41 bp, Artificial
  Primer
taacatcaccatctaattcaacaagaattgggacaactcca

SEQ ID NO: 130 - DNA, 36 bp, Artificial
  Primer
cttcaccctctccactgacagaaaatttgtgcccat

SEQ ID NO: 131 - DNA, 41 bp, Artificial
  Primer
gcaaataaatttaagggtaagttttccgtatgttgcatcac

SEQ ID NO: 132 - DNA, 37 bp, Artificial
  Primer
caagtgttggccatggaacaggtagttttccagtagt

SEQ ID NO: 133 - DNA, 38 bp, Artificial
  Primer
tctcgcaaagcattgaacaccataaccgaaagtagtga

SEQ ID NO: 134 - DNA, 41 bp, Artificial
  Primer
gcactcttgaaaaagtcatgctgtttcatatgatctgggta

SEQ ID NO: 135 - DNA, 40 bp, Artificial
  Primer
gaaaaatatagttctttcctgtacataaccttcgggcatg

SEQ ID NO: 136 - DNA, 36 bp, Artificial
  Primer
gacttcagcacgtgtcttgtagttcccgtcatcttt

SEQ ID NO: 137 - DNA, 43 bp, Artificial
  Primer
cttttaactcgattctattaacaagggtatcaccttcaaactt

SEQ ID NO: 138 - DNA, 44 bp, Artificial
  Primer
caatttgtgtccaagaatgtttccatcttctttaaaatcaatac

SEQ ID NO: 139 - DNA, 43 bp, Artificial
  Primer
tgtctgccatgatgtatacattgtgtgagttatagttgtattc

SEQ ID NO: 140 - DNA, 46 bp, Artificial
  Primer
atgttgtgtctaattttgaagttaactttgattccattcttttgtt

SEQ ID NO: 141 - DNA, 38 bp, Artificial
  Primer
gttgataatggtctgctagttgaacgcttccatcttca

SEQ ID NO: 142 - DNA, 37 bp, Artificial
  Primer
ggtaaaaggacagggccatcgccaattggagtatttt

SEQ ID NO: 143 - DNA, 36 bp, Artificial
  Primer
gaaagggcagattgtgtggacaggtaatggttgtct

SEQ ID NO: 144 - DNA, 37 bp, Artificial
  Primer
aaggaccatgtggtctctcttttcgttgggatctttc

SEQ ID NO: 145 - DNA, 37 bp, Artificial
  Primer
tgccatgtgtaatcccagcagctgttacaaactcaag

SEQ ID NO: 146 - DNA, 41 bp, Artificial
  Primer
cgaccgagaagaagtaagcttttatttgtatagttcatcca

SEQ ID NO: 147 - DNA, 35 bp, Artificial
  Primer
gaaagcagtcaccctatccgctgatcagcctcatg

SEQ ID NO: 148 - DNA, 25 bp, Artificial
  Primer
gatagggtgactgctttcgcgtaca

SEQ ID NO: 149 - DNA, 25 bp, Artificial
  Primer
cagcctcatgcgaccgagaagaagt

SEQ ID NO: 150 - DNA, 39 bp, Artificial
  Primer
gatcggtaccatgagtaaaggagaagaacttttcactgg

SEQ ID NO: 151 - DNA, 39 bp, Artificial
  Primer
gatcaagcttttatttgtatagttcatccatgccatgtg

SEQ ID NO: 152 - DNA, 18 bp, Artificial
  Primer
gatagggtgactgctttc

SEQ ID NO: 153 - DNA, 35 bp, Artificial
  Primer
aagcttttatttgtatagttcatccatgccatgtg

SEQ ID NO: 154 - DNA, 779 bp, Artificial
  Synthesized GFPmut3 sequence
gatagggtgactgctttcgcgtacaggtaccatgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcggttatggtgttcaatgctttgcgagatacccagatcatatgaaacagcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaataaaagcttacttcttctcggtcgcatgaggctg

SEQ ID NO: 155 - DNA, 20 bp, Artificial
  Primer
atatagatgccgtcctagcg

SEQ ID NO: 156 - DNA, 20 bp, Artificial
  Primer
aagtatctttcctgtgccca

SEQ ID NO: 157 - DNA, 20 bp, Artificial
  Primer
ccctttaatcagatgcgtcg

SEQ ID NO: 158 - DNA, 20 bp, Artificial
  Primer
tggtagtaataagggcgacc

SEQ ID NO: 159 - DNA, 20 bp, Artificial
  Primer
aatccttgcgtcaatggttc

SEQ ID NO: 160 - DNA, 20 bp, Artificial
  Primer
gggttctcggattttacacg

SEQ ID NO: 161 - DNA, 20 bp, Artificial
  Primer
tgtcgtgcctctttatctgt

SEQ ID NO: 162 - DNA, 20 bp, Artificial
  Primer
gcttcggtgtatcggaaatg

SEQ ID NO: 163 - DNA, 20 bp, Artificial
  Primer
atttaaacggtgaggtgtgc

SEQ ID NO: 164 - DNA, 20 bp, Artificial
  Primer
tatcgtttcgctggctatca

SEQ ID NO: 165 - DNA, 20 bp, Artificial
  Primer
gttcaatcactgaatcccgg

SEQ ID NO: 166 - DNA, 20 bp, Artificial
  Primer
gtcgagtcctatgtaaccgt

SEQ ID NO: 167 - DNA, 20 bp, Artificial
  Primer
caggggtcgtcatatcttca

SEQ ID NO: 168 - DNA, 20 bp, Artificial
  Primer
gtaagatggaagccgggata

SEQ ID NO: 169 - DNA, 20 bp, Artificial
  Primer
cacctcatagagctgtggaa

SEQ ID NO: 170 - DNA, 20 bp, Artificial
  Primer
cttaaaccggccaacatacc

SEQ ID NO: 171 - DNA, 20 bp, Artificial
  Primer
tgctctttattcgttgcgtc

SEQ ID NO: 172 - DNA, 20 bp, Artificial
  Primer
tgagccttatgatttcccgt

SEQ ID NO: 173 - DNA, 20 bp, Artificial
  Primer
cgttctaaacggctagatgc

SEQ ID NO: 174 - DNA, 20 bp, Artificial
  Primer
gtatccgaagcgtggagtat

SEQ ID NO: 175 - DNA, 20 bp, Artificial
  Primer
cttgttatggacgagttgcc

SEQ ID NO: 176 - DNA, 20 bp, Artificial
  Primer
ccaaagattcaaccgtcctg

SEQ ID NO: 177 - DNA, 20 bp, Artificial
  Primer
tattcatgcttggacggact

SEQ ID NO: 178 - DNA, 20 bp, Artificial
  Primer
atcgacaatggtatggctga

SEQ ID NO: 179 - DNA, 20 bp, Artificial
  Primer
gtcctagtgaggaataccgg

SEQ ID NO: 180 - DNA, 20 bp, Artificial
  Primer
ttagataggtgtgtaggcgc

SEQ ID NO: 181 - DNA, 20 bp, Artificial
  Primer
ttccgtttatgctttccagc

SEQ ID NO: 182 - DNA, 20 bp, Artificial
  Primer
gtatagtttgtgcggtggtc

SEQ ID NO: 183 - DNA, 20 bp, Artificial
  Primer
tcagcctttcattgattgcg

SEQ ID NO: 184 - DNA, 20 bp, Artificial
  Primer
agggtcgtggttaaaggtac

SEQ ID NO: 185 - DNA, 20 bp, Artificial
  Primer
tgcaagtgtacaaatccagc

SEQ ID NO: 186 - DNA, 20 bp, Artificial
  Primer
cttaaggtttgcccattccc

SEQ ID NO: 187 - DNA, 20 bp, Artificial
  Primer
tggttcgttagtcgatctcc

SEQ ID NO: 188 - DNA, 20 bp, Artificial
  Primer
tattttgtagagcgttcgcg

SEQ ID NO: 189 - DNA, 20 bp, Artificial
  Primer
ttctgtaagtttcgtcggga

SEQ ID NO: 190 - DNA, 20 bp, Artificial
  Primer
ttgacgtacgtaggttctcc

SEQ ID NO: 191 - DNA, 20 bp, Artificial
  Primer
gagatgagtagacgagtggg

SEQ ID NO: 192 - DNA, 20 bp, Artificial
  Primer
ctttgggctttcagatgagc

SEQ ID NO: 193 - DNA, 20 bp, Artificial
  Primer
tgtcatatgctaacgtccgt

SEQ ID NO: 194 - DNA, 20 bp, Artificial
  Primer
ttgcgacatcacaattctcg

SEQ ID NO: 195 - DNA, 20 bp, Artificial
  Primer
tcagtatggcgtcttgaagt

SEQ ID NO: 196 - DNA, 20 bp, Artificial
  Primer
tcatgtcgtgaccagtagac

SEQ ID NO: 197 - DNA, 20 bp, Artificial
  Primer
aactaacggatttaagcgcg

SEQ ID NO: 198 - DNA, 20 bp, Artificial
  Primer
cattttctgttccccagtgg

SEQ ID NO: 199 - DNA, 20 bp, Artificial
  Primer
atttgcctaaccactccact

SEQ ID NO: 200 - DNA, 20 bp, Artificial
  Primer
tgacttatgaacctttgcgc

SEQ ID NO: 201 - DNA, 20 bp, Artificial
  Primer
ataggattagctgatgggcc

SEQ ID NO: 202 - DNA, 20 bp, Artificial
  Primer
tgagattcgggactattcgg

SEQ ID NO: 203 - DNA, 20 bp, Artificial
  Primer
ttggttagtacacgggactc

SEQ ID NO: 204 - DNA, 20 bp, Artificial
  Primer
atttgtgtatcgaggctcgt

SEQ ID NO: 205 - DNA, 20 bp, Artificial
  Primer
atcgttccccatcacattct

SEQ ID NO: 206 - DNA, 20 bp, Artificial
  Primer
attaccatgttatcgggcga

SEQ ID NO: 207 - DNA, 20 bp, Artificial
  Primer
tcggtggatatgacgtaacc

SEQ ID NO: 208 - DNA, 20 bp, Artificial
  Primer
ggtcagatggtttacatgcg

SEQ ID NO: 209 - DNA, 20 bp, Artificial
  Primer
tctcgttcgaaaatcatcgc

SEQ ID NO: 210 - DNA, 20 bp, Artificial
  Primer
tgcaaatgtgaggtagcaac

SEQ ID NO: 211 - DNA, 20 bp, Artificial
  Primer
aaagtcaaagtgcgtttcgt

SEQ ID NO: 212 - DNA, 20 bp, Artificial
  Primer
atgctactcgttcctttcga

SEQ ID NO: 213 - DNA, 20 bp, Artificial
  Primer
tcttatcggtgcttcgttct

SEQ ID NO: 214 - DNA, 20 bp, Artificial
  Primer
gtccgttttcctgaatgagc

SEQ ID NO: 215 - DNA, 20 bp, Artificial
  Primer
agtctgtctttcccctttcc

SEQ ID NO: 216 - DNA, 20 bp, Artificial
  Primer
caggtatgcgtaggagtcaa

SEQ ID NO: 217 - DNA, 20 bp, Artificial
  Primer
ttaatggcgcgttcatactg

SEQ ID NO: 218 - DNA, 20 bp, Artificial
  Primer
attagccatttcaggacgga

SEQ ID NO: 219 - DNA, 20 bp, Artificial
  Primer
actatgtaccgcttgttgga

SEQ ID NO: 220 - DNA, 20 bp, Artificial
  Primer
tatgtctcctagccactcct

SEQ ID NO: 221 - DNA, 20 bp, Artificial
  Primer
ccgaagaatcgcagatccta

SEQ ID NO: 222 - DNA, 20 bp, Artificial
  Primer
taaggtgcgtactagctgac

SEQ ID NO: 223 - DNA, 20 bp, Artificial
  Primer
tccttggagtttagagcgag

SEQ ID NO: 224 - DNA, 20 bp, Artificial
  Primer
atcaatcccctacaccttcg

SEQ ID NO: 225 - DNA, 20 bp, Artificial
  Primer
ttccttgataccgtagctcg

SEQ ID NO: 226 - DNA, 20 bp, Artificial
  Primer
cgtttctttccggtcgttag

SEQ ID NO: 227 - DNA, 20 bp, Artificial
  Primer
gaacggtgatccctttccta

SEQ ID NO: 228 - DNA, 20 bp, Artificial
  Primer
tgttatagcttccacggtgt

SEQ ID NO: 229 - DNA, 20 bp, Artificial
  Primer
agacgggattttactgggtc

SEQ ID NO: 230 - DNA, 20 bp, Artificial
  Primer
tctttgcttcgcaagtcttg

SEQ ID NO: 231 - DNA, 20 bp, Artificial
  Primer
ctaaacaccgcacctcacta

SEQ ID NO: 232 - DNA, 20 bp, Artificial
  Primer
gaacacaactacactgacgc

SEQ ID NO: 233 - DNA, 20 bp, Artificial
  Primer
atggtcactgactcgcatta

SEQ ID NO: 234 - DNA, 20 bp, Artificial
  Primer
caaagatttctgtcggtcgg

SEQ ID NO: 235 - DNA, 20 bp, Artificial
  Primer
tggctactttcttagcggaa

SEQ ID NO: 236 - DNA, 20 bp, Artificial
  Primer
tacttcgagacttcatgcgt

SEQ ID NO: 237 - DNA, 20 bp, Artificial
  Primer
atggcccgacctctattatg

SEQ ID NO: 238 - DNA, 20 bp, Artificial
  Primer
tgggtctagtgaacttcgtc

SEQ ID NO: 239 - DNA, 20 bp, Artificial
  Primer
aacatatgttgcttcgtccg

SEQ ID NO: 240 - DNA, 20 bp, Artificial
  Primer
tcgagttagattgtcacccc

SEQ ID NO: 241 - DNA, 20 bp, Artificial
  Primer
tcagagcttttcggtacagt

SEQ ID NO: 242 - DNA, 20 bp, Artificial
  Primer
gcccaggagtagtcgttaat

SEQ ID NO: 243 - DNA, 20 bp, Artificial
  Primer
tctgtgttccgactaaggtc

SEQ ID NO: 244 - DNA, 20 bp, Artificial
  Primer
tctgttgttagactccgacc

SEQ ID NO: 245 - DNA, 20 bp, Artificial
  Primer
gtacgtctgaacttgggact

SEQ ID NO: 246 - DNA, 20 bp, Artificial
  Primer
agacacgcgattgtttaacc

SEQ ID NO: 247 - DNA, 20 bp, Artificial
  Primer
ccgttcgttttgagcactta

SEQ ID NO: 248 - DNA, 20 bp, Artificial
  Primer
aggttagggaacgcaagatt

SEQ ID NO: 249 - DNA, 20 bp, Artificial
  Primer
ccagactgtgctcgttatct

SEQ ID NO: 250 - DNA, 20 bp, Artificial
  Primer
agttgttctctatccgcgat

SEQ ID NO: 251 - DNA, 20 bp, Artificial
  Primer
gattaaatctcgccggtgac

SEQ ID NO: 252 - DNA, 20 bp, Artificial
  Primer
ttgtagttttcgcttgcgtt

SEQ ID NO: 253 - DNA, 20 bp, Artificial
  Primer
tgtgttgctctctcatagcc

SEQ ID NO: 254 - DNA, 20 bp, Artificial
  Primer
gcttattcgtgccgtgttat

SEQ ID NO: 255 - DNA, 20 bp, Artificial
  Primer
tttgcttcagtcagattcgc

SEQ ID NO: 256 - DNA, 20 bp, Artificial
  Primer
gtcgagtcctatgtaaccgt

SEQ ID NO: 257 - DNA, 20 bp, Artificial
  Primer
gtaagatggaagccgggata

SEQ ID NO: 258 - DNA, 20 bp, Artificial
  Primer
ggtgtcgcaacatgatctac

SEQ ID NO: 259 - DNA, 20 bp, Artificial
  Primer
gtgctaagtcacactgttgg

SEQ ID NO: 260 - DNA, 20 bp, Artificial
  Primer
tctaaacagttaggcccagg

SEQ ID NO: 261 - DNA, 20 bp, Artificial
  Primer
gtctttatacttgcctgccg

SEQ ID NO: 262 - DNA, 20 bp, Artificial
  Primer
caccgcgatcaatacaactt

SEQ ID NO: 263 - DNA, 20 bp, Artificial
  Primer
ttcggatagactcaggaagc

SEQ ID NO: 264 - DNA, 20 bp, Artificial
  Primer
ccattgatagattcgctcgc

SEQ ID NO: 265 - DNA, 20 bp, Artificial
  Primer
ttttctactttccggcttgc

SEQ ID NO: 266 - DNA, 20 bp, Artificial
  Primer
atgactattggggtcgtacc

SEQ ID NO: 267 - DNA, 20 bp, Artificial
  Primer
tcgacaatagttgagccctt

SEQ ID NO: 268 - DNA, 20 bp, Artificial
  Primer
gagccatgtgaaatgtgtgt

SEQ ID NO: 269 - DNA, 20 bp, Artificial
  Primer
cgtatacgtaagggttccga

SEQ ID NO: 270 - DNA, 20 bp, Artificial
  Primer
ttatgatgtccggatacccg

SEQ ID NO: 271 - DNA, 20 bp, Artificial
  Primer
tcttagaaatccacgggtcc

SEQ ID NO: 272 - DNA, 20 bp, Artificial
  Primer
gaagggtggatcatcgtact

SEQ ID NO: 273 - DNA, 20 bp, Artificial
  Primer
ggctgttagttttagagccg

SEQ ID NO: 274 - DNA, 20 bp, Artificial
  Primer
agtggtgtagtggcttctac

SEQ ID NO: 275 - DNA, 20 bp, Artificial
  Primer
ctcagagggagttcaactgt

SEQ ID NO: 276 - DNA, 20 bp, Artificial
  Primer
tttggcagatcattaacggc

SEQ ID NO: 277 - DNA, 20 bp, Artificial
  Primer
tatgatctccgtacacgagc

SEQ ID NO: 278 - DNA, 20 bp, Artificial
  Primer
agtgccatgttatccctgaa

SEQ ID NO: 279 - DNA, 20 bp, Artificial
  Primer
ttatacatctggacgcctcc

SEQ ID NO: 280 - DNA, 20 bp, Artificial
  Primer
tcctcgattctccaatcagg

SEQ ID NO: 281 - DNA, 20 bp, Artificial
  Primer
gcttaacgcatttcaagcac

SEQ ID NO: 282 - DNA, 20 bp, Artificial
  Primer
cttttatgttcctcgcaggg

SEQ ID NO: 283 - DNA, 20 bp, Artificial
  Primer
gtgggcgttagcaaattaca

SEQ ID NO: 284 - DNA, 20 bp, Artificial
  Primer
agagattattaggcgtgggg

SEQ ID NO: 285 - DNA, 20 bp, Artificial
  Primer
taggattactgctcggtgac

SEQ ID NO: 286 - DNA, 20 bp, Artificial
  Primer
tcgcgtgagtggttcatata

SEQ ID NO: 287 - DNA, 20 bp, Artificial
  Primer
caatagatacccacccgtca

SEQ ID NO: 288 - DNA, 20 bp, Artificial
  Primer
atatatccgccgttgtacgt

SEQ ID NO: 289 - DNA, 20 bp, Artificial
  Primer
cgagagtctcccacgatatc

SEQ ID NO: 290 - DNA, 20 bp, Artificial
  Primer
attcagttggtcttacgggt

SEQ ID NO: 291 - DNA, 20 bp, Artificial
  Primer
ggattgcaacgtcaggaaat

SEQ ID NO: 292 - DNA, 20 bp, Artificial
  Primer
gaatgttgcagactggaagg

SEQ ID NO: 293 - DNA, 20 bp, Artificial
  Primer
gtccatgaatacaacaccgg

SEQ ID NO: 294 - DNA, 20 bp, Artificial
  Primer
tcgaacaatttgcgataccc

SEQ ID NO: 295 - DNA, 20 bp, Artificial
  Primer
aagtgcacatttcgtttcga

SEQ ID NO: 296 - DNA, 20 bp, Artificial
  Primer
tacttttgattgctgtgccc

SEQ ID NO: 297 - DNA, 20 bp, Artificial
  Primer
gttcaatcactgaatcccgg

SEQ ID NO: 298 - DNA, 20 bp, Artificial
  Primer
caggggtcgtcatatcttca

SEQ ID NO: 299 - DNA, 20 bp, Artificial
  Primer
cacctcatagagctgtggaa

SEQ ID NO: 300 - DNA, 20 bp, Artificial
  Primer
cggttcctagtcatgtttgc

SEQ ID NO: 301 - DNA, 20 bp, Artificial
  Primer
ttgtactaatctcgtcccgg

SEQ ID NO: 302 - DNA, 20 bp, Artificial
  Primer
ttatgttcacaactggcgtg

SEQ ID NO: 303 - DNA, 20 bp, Artificial
  Primer
tggaactgatttggcctttg

SEQ ID NO: 304 - DNA, 20 bp, Artificial
  Primer
tatagttcctcccatgcacc

SEQ ID NO: 305 - DNA, 20 bp, Artificial
  Primer
acaatagacagacccatgca

SEQ ID NO: 306 - DNA, 20 bp, Artificial
  Primer
gagtcgagctagcataggag

SEQ ID NO: 307 - DNA, 20 bp, Artificial
  Primer
ttgtgggagcttcttaccat

SEQ ID NO: 308 - DNA, 20 bp, Artificial
  Primer
tcgtacgggaatgaccatag

SEQ ID NO: 309 - DNA, 20 bp, Artificial
  Primer
agacacaacgtagccgatta

SEQ ID NO: 310 - DNA, 20 bp, Artificial
  Primer
cggactaaaggatcgagtca

SEQ ID NO: 311 - DNA, 20 bp, Artificial
  Primer
catcggataacacaaagcgt

SEQ ID NO: 312 - DNA, 20 bp, Artificial
  Primer
gatgtatactccaccgtggt

SEQ ID NO: 313 - DNA, 20 bp, Artificial
  Primer
tgagatatgtacctggtgcc

SEQ ID NO: 314 - DNA, 20 bp, Artificial
  Primer
attcttgggcctatcgttgt

SEQ ID NO: 315 - DNA, 20 bp, Artificial
  Primer
aaaccatatacagccgtcgt

SEQ ID NO: 316 - DNA, 20 bp, Artificial
  Primer
tagctaaatcccacccgatg

SEQ ID NO: 317 - DNA, 20 bp, Artificial
  Primer
gtgcggttacagttttgact

SEQ ID NO: 318 - DNA, 20 bp, Artificial
  Primer
gggactacatagggtgacag

SEQ ID NO: 319 - DNA, 20 bp, Artificial
  Primer
cgttgtcgttccaaagaagt

SEQ ID NO: 320 - DNA, 20 bp, Artificial
  Primer
agtcacacatatacggaccc

SEQ ID NO: 321 - DNA, 20 bp, Artificial
  Primer
agagaacccctattatggcg

SEQ ID NO: 322 - DNA, 20 bp, Artificial
  Primer
tcgttaggctaaaacatgcg

SEQ ID NO: 323 - DNA, 20 bp, Artificial
  Primer
tgataggtcgttcagcctac

SEQ ID NO: 324 - DNA, 20 bp, Artificial
  Primer
tcgggactttcataagcact

SEQ ID NO: 325 - DNA, 20 bp, Artificial
  Primer
attttatgcgtccagttcgg

SEQ ID NO: 326 - DNA, 20 bp, Artificial
  Primer
aaggctggtatttcccttca

SEQ ID NO: 327 - DNA, 20 bp, Artificial
  Primer
catactgttggttgctaggc

SEQ ID NO: 328 - DNA, 20 bp, Artificial
  Primer
atatactggattccgccgtt

SEQ ID NO: 329 - DNA, 20 bp, Artificial
  Primer
acttatgaacccttggcact

SEQ ID NO: 330 - DNA, 20 bp, Artificial
  Primer
atagatgtatgccgttcggt

SEQ ID NO: 331 - DNA, 20 bp, Artificial
  Primer
tctctgttttccgcactttg

SEQ ID NO: 332 - DNA, 20 bp, Artificial
  Primer
agttattcgtctttcccggt

SEQ ID NO: 333 - DNA, 20 bp, Artificial
  Primer
tacaggaatctccacgaagc

SEQ ID NO: 334 - DNA, 20 bp, Artificial
  Primer
cctcgggcttgttactagat

SEQ ID NO: 335 - DNA, 20 bp, Artificial
  Primer
attcttccgtccaacgtact

SEQ ID NO: 336 - DNA, 20 bp, Artificial
  Primer
taatcatacgagtgggcctc

SEQ ID NO: 337 - DNA, 20 bp, Artificial
  Primer
agttggtagaattgaccggt

SEQ ID NO: 338 - DNA, 723 bp, Artificial
  Primer
ggtaccatggtgagcaagggcgaggaaaccacaatgggcgtaatcaagcccgacatgaagatcaagctgaagatggagggcaacgtgaatggccacgccttcgtgatcgagggcgagggcgagggcaagccctacgacggcaccaacaccatcaacctggaggtgaaggagggagcccccctgcccttctcctacgacattctgaccaccgcgttcgcctacggcaacagggccttcaccaagtaccccgacgacatccccaactacttcaagcagtccttccccgagggctactcttgggagcgcaccatgaccttcgaggacaagggcatcgtgaaggtgaagtccgacatctccatggaggaggactccttcatctacgagatacacctcaagggcgagaacttcccccccaacggccccgtgatgcagaaaaagaccaccggctgggacgcctccaccgagaggatgtacgtgcgcgacggcgtgctgaagggcgacgtcaagcacaagctgctgctggagggcggcggccaccaccgcgttgacttcaagaccatctacagggccaagaaggcggtgaagctgcccgactatcactttgtggaccaccgcatcgagatcctgaaccacgacaaggactacaacaaggtgaccgtttacgagagcgccgtggcccgcaactccaccgacggcatggacgagctgtacaagtaaaagctt

SEQ ID NO: 339 - DNA, 732 bp, Artificial
  Primer
ggtaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccaaactgagcaaagaccccaacgagaagcgcgatcacatggtcctgctggagttcgtgaccgccgccgggatcactctcggcatggacgagctgtacaagtaaaagctt

SEQ ID NO: 340 - DNA, 723 bp, Artificial
  Primer
ggtaccatggtgagcaagggcgaggagaataacatggccatcatcaaggagttcatgcgcttcaaggtgcacatggagggctccgtgaacggccacgagttcgagatcgagggcgagggcgagggccgcccctacgaggcctttcagaccgctaagctgaaggtgaccaagggtggccccctgcccttcgcctgggacatcctgtcccctcagttcatgtacggctccaaggtctacattaagcacccagccgacatccccgactacttcaagctgtccttccccgagggcttcaggtgggagcgcgtgatgaacttcgaggacggcggcattattcacgttaaccaggactcctccctgcaggacggcgtgttcatctacaaggtgaagctgcgcggcaccaacttcccctccgacggccccgtaatgcagaaaaagaccatgggctgggaggcctccgaggagcggatgtaccccgaggacggcgccttaaagagcgagatcaaaaagaggctgaagctgaaggacggcggccactacgccgccgaggtcaagaccacctacaaggccaagaagcccgtgcagctgcccggcgcctacatcgtcgacatcaagttggacatcgtgtcccacaacgaggactacaccatcgtggaacagtacgaacgcgccgagggccgccactccaccggcggcatggacgagctgtacaagtaaaagctt

SEQ ID NO: 341 - DNA, 757 bp, Artificial
  Primer
ggcccagccggccaggcgcgaagtgcagctggtggagtcaggcggtggactggtgcagccaggaggttccctgagactctcatgcgcagcaagcggttttaatatcaaggacacttatatacactgggtgcgccaagcccccggaaagggtctggagtgggtggccagaatataccccacaaacggctataccaggtacgcagattcagtgaaggggagattcaccataagcgctgacacatctaagaatactgcttacctgcaaatgaattccctgagggcagaggatacagctgtttattactgcagccggtggggcggagatggcttttacgccatggactattgggggcagggaaccctggtcaccgtttccagcggtgggtcagggggcagcggcggcgccagcggagcagggagcggtggaggcgatatccaaatgacacagtccccctctagcctgagcgccagcgtcggtgacagggtgaccattacatgcagggcctctcaggatgttaatactgccgttgcatggtaccagcagaagcccgggaaggcaccaaagctgctgatctattccgcttcctttctgtacagcggagtgcctagcaggttttccggatctcgcagcggaactgattttacactcaccatcagcagcctccaacctgaggattttgccacctattattgccagcaacactacaccactccacccactttcggccagggaactaaggtggaaataaaagggccc

SEQ ID NO: 342 - DNA, 754 bp, Artificial
  Primer
ggcccagccggccaggcgccaggttcagctcaagcagtctggacccggactggtgcagccctctcagtctctctctatcacctgcacagtgtctggtttctctctcaccaactacggggtccattgggttcggcagtccccagggaaagggctcgaatggctgggcgtgatctggtccggcggcaataccgactacaacaccccatttacttccaggctgtcaattaataaggacaattctaagagccaggtcttctttaagatgaactctctccagtctaatgatactgccatctactactgtgcccgggcactcacatactacgattatgaattcgcttactggggccagggcaccctcgtcaccgtgagcgcaggaggatctgctggctctgggtcaagcggtggcgcttccggctcagggggagacatcctgctcacccagagccccgtgattctgtccgttagccccggagaacgcgtttcttttagctgtcgcgcatctcagagcatcggtaccaacattcactggtatcagcagcggaccgacgggagccctcgcctcctgataaaatatgcttctgagtcaattagcggtatcccctccagatttagcgggagcggttctgggaccgatttcacactgagcatcaactctgtggagtctgaagatatcgctgattattactgtcagcaaaacaacaattggcctaccaccttcggcgccggcaccaagctggaactgaaagggccc

SEQ ID NO: 343 - DNA, 760 bp, Artificial
  Primer
ggcccagccggccaggcgccaagttcagctccaggagtcaggtcctggtctggtgagaccatcccagaccctctctctcacttgtaccgtttccggcttcacattcaccgatttctatatgaactgggttaggcaaccaccaggccgggggctggaatggatcggttttatcagagataaagccaagggatatactactgagtacaacccctctgtgaagggtcgggtgaccatgctggttgacacaagcaagaatcaattttcactccggctgtcatctgtgacagctgctgatacagcagtttattattgcgcaagggaaggacatactgccgctcctttcgactattggggccagggttcactcgtcacagtctcttcaggtggggccggctcaggagccgggagcgggtcatctggagccggctccggggatatccagatgacccagtcaccctcttcactcagcgccagcgtgggcgatcgcgttaccatcacatgcaaagcttctcagaacattgacaaatacctgaattggtaccaacagaagcccggcaaggcccccaaactcctcatatacaatacaaacaatctgcagaccggcgtgccatcccgcttctcaggatcaggcagcggcactgactttactttcacaatcagcagcctgcaaccagaggacatcgccacatattactgtctccagcatatctcccgccctcggacattcggccaaggtacaaaggtggagattaaagggccc

SEQ ID NO: 344 - DNA, 757 bp, Artificial
  Primer
ggcccagccggccaggcgcgaagtgcaactggttgaaagcggtgggggcctggtgcagcctggtggatcactgagactctcctgcgccgccagcggttacaccttcaccaactatggtatgaattgggttagacaagcacctggaaagggactggagtgggttggctggataaatacatatacaggcgagccaacatatgcagctgactttaagcggaggtttaccttctcactggacacatccaagtctactgcttacctgcagatgaactcactccgggctgaggatacagccgtttactattgcgccaagtatccccattactatggttccagccactggtacttcgatgtctggggccagggaactctggtgactggggggtccgggggctccggaggggcctccggagcaggatccggcggaggtgacatacagatgacccagtctccatcctctctgagcgcctctgtgggcgatcgcgtcactattacctgttctgcatctcaggatattagcaactatctgaattggtatcagcagaagccaggtaaggcaccaaaagttctgatctacttcacaagctctctgcattccggggtgccctcacgcttctctggttccggctccgggacagatttcacactcacaatttcctctctgcagcccgaagattttgcaacttactactgtcagcagtattctacagtgccatggactttcggacagggaaccaaggtcgagattaaagggccc

SEQ ID NO: 345 - DNA, 766 bp, Artificial
  Primer
ggcccagccggccaggcgcgaagttcagctggttgaaagcggaggtggactcgtgcagcccggtgggtccctgaggctctcctgcgccgctagcggatatgatttcactcactacggtatgaattgggtccggcaggctcccggcaaaggtctggaatgggttggctggatcaacacttatactggggagcctacctacgccgccgatttcaagaggcgctttactttctcactcgatacctccaaatccacagcctatctgcaaatgaattccctgcgcgccgaagataccgcagtctactattgtgccaagtatccctactattatgggacatctcactggtacttcgacgtgtgggggcaagggactctcgtcactgtgtctagcgggggtagcgctgggtccggcagcagcggtggggcaagcggtagcgggggcgacattcagctgacacaaagcccctcatccctgagcgcttcagtgggggaccgcgtgaccatcacctgttccgcctcccaggacatctcaaactacctgaactggtaccaacaaaaacctggtaaagcccctaaagttctgatttacttcacaagctctctccactccggcgtcccttctaggttttctggtagcggtagcggaacagatttcactctgacaattagctccctccagcctgaggattttgccacttactattgtcagcagtattccacagtgccctggacttttgggcagggcaccaaggtcgaaatcaaggggccc

SEQ ID NO: 346 - DNA, 757 bp, Artificial
  Primer
ggcccagccggccaggcgcgaggtccagctggtcgagagcggcggcgggctggttcaacccgggggctccctgcggctgtcatgtgccgccagcggcttcacctttactgattacacaatggactgggtgaggcaggccccaggaaaaggcctggaatgggttgccgacgtgaatcctaattccgggggttcaatttacaatcagcgctttaagggccggttcaccctgtcagtcgacaggagcaagaatacactctatctccagatgaactccctccgcgctgaggataccgccgtctattattgtgcccgcaatctgggtccctctttttactttgactattggggccaagggaccctggtcaccgtctctagcgccggtggctcaggaggaagcggtggcgcctctggggctggcagcggaggaggcgacattcagatgacacagagccctagctctctctccgctagcgtgggggacagggttaccataacttgcaaggcaagccaagatgtctctattggtgttgcttggtaccagcaaaagcctggaaaggctcctaaactgctgatatactccgccagctacaggtatacaggcgtgccatcccggttctcaggttccggctcaggaacagattttactctcaccatttccagcctgcaacccgaggacttcgccacatactattgccagcagtattatatatatccttacacttttggtcagggtactaaagtggagattaaagggccc

SEQ ID NO: 347 - DNA, 757 bp, Artificial
  Primer
ggcccagccggccaggcgcgaggtgcagctccaacaatctgggcctgatctggttaagccaggcgcttctgtgaaaatttcctgtaaggcttcaggctacagcttcactggctattatatgcattgggtgaaacagtctccaggaaagggcctggagtggattgggcggatcaatcccaacaatggagtcaccctctacaatcaaaaattcaaagataaagctacactgaccgtcgataaaagctcaacaacagcctacatggagctgagatccctcacctccgaggacagcgctgtctactactgcgccaggtccacaatgattaccaattatgtgatggactactggggtcagggaacctcagtgaccgttagctctggcgggtccgcaggtagcggctcatccggcggcgcatccgggagcggagggtctattgtcatgacacagacccccacttccctcctggtctctgctggcgacagagtcacaatcacttgcaaggctagccagagcgtttcaaacgacgtggcatggtatcaacagaaacccggccaatcccccaaactgctgatttcttacacatcatccagatacgccggtgtgcccgataggttttctggttcagggtatggaactgacttcactctcactatctctagcgttcaggctgaagacgctgccgtctacttctgccagcaagactacaactctcctcctacattcggcgggggcacaaagctggagatcaaagggccc

SEQ ID NO: 348 - DNA, 754 bp, Artificial
  Primer
ggcccagccggccaggcgccaggtgcagctggtgcagtccggagccgaggtcaagaagcccggatcttccgtcaaagtcagctgcaaagcttccggttatgcattcactaactacctcatcgagtgggtccgccaggctccaggacagggactggagtggattggagtgatctaccctggatcaggaggcacaaattataacgagaagtttaagggcagagtcactctgaccgtcgatgaatccacaaatacagcttacatggagctgtcatcactccggagcgaggacacagcagtttatttttgcgcacgccgcgatggcaattacgggtggttcgcctattgggggcagggtactctcgtcaccgtgtcatcaggtggggctggctccggggcaggttctggctcctccggagctggttcaggagacatccagatgacccagacaccctccactctctctgcttctgtgggagacagagtcacaatcagctgccgggcttcccaggatataaacaactacctgaactggtaccagcagaagcctgggaaggcccccaagctgctgatctactatacatccactctgcacagcggagttcctagccgcttcagcggatccggtagcgggaccgactataccctgaccatctcaagcctgcagcccgatgacttcgccacatacttctgtcagcagggaaacaccctcccatggacattcggtcaaggaactaaagttgaggttaaagggccc

SEQ ID NO: 349 - DNA, 757 bp, Artificial
  Primer
ggcccagccggccaggcgcgaagttcaactggttgagagcggtgccgaggtgaagaagcctggagagtctctgagaattagctgtaagggctctggctgcatcatctcatcttattggatttcatgggttagacagatgcccggcaaaggactggaatggatgggcaagatagaccctggtgactcctacatcaattattccccttcttttcaggggcatgtcacaatctccgcagacaagagcatcaacacagcatatctccagtggaattcactgaaagcctccgacacagccatgtactattgcgcaagaggagggagggacttcggagactcttttgactactgggggcaggggactctggtgacagtgtctagcggcgggtcaggaggatccggtggagcctctggcgctggaagcggcggcggagatgtggtcatgactcaatccccttcctttctgtcagcattcgtgggcgataggatcactattacttgtcgcgcctcttctggcatctccagatatctggcttggtaccagcaagctcccggaaaggcccctaagctgctcatatatgccgcctccaccctccagactggagtgcccagccggtttagcggtagcggttccggtaccgagtttaccctcaccattaactctctgcagccagaagacttcgccacatattactgtcaacacctcaactcctatcctctcactttcggcggcgggaccaaagtcgatattaaggggccc

SEQ ID NO: 350 - DNA, 754 bp, Artificial
  Primer
ggcccagccggccaggcgccaagttaaactgcaggagagcggagccgaactcgccagacccggagcttctgtgaaactgagctgcaaagcttctggctatacttttaccaattattggatgcaatgggtgaagcagaggccaggacagggactggactggatcggagctatctatcctggagacggcaatactcggtacacacacaaatttaaggggaaagctaccctgaccgctgataagtcatcatctaccgcctacatgcagctgagctccctggcttcagaggacagcggcgtttactattgcgcacgcggcgagggaaactatgcatggtttgcatactgggggcaggggaccaccgtgactgtgtcctcaggggggagcgctggtagcggttccagcggcggggccagcggttccgggggggacatcgagctcactcagtctcctgcaagcctgtcagcatcagttggggagacagttaccatcacctgccaggcatccgaaaatatatacagctacctcgcatggcatcagcaaaagcagggtaaaagccctcagctcctggtttataatgctaaaaccctggctggaggcgtctcttcaagatttagcgggagcggctccgggacccacttctcactgaaaataaagtccctgcaaccagaggattttggtatttactattgtcagcaccactacggcatactcccaaccttcggagggggaactaagctggaaatcaaggggccc

SEQ ID NO: 351 - DNA, 754 bp, Artificial
  Primer
ggcccagccggccaggcgccaggttaccctgcgcgagagcgggcctgctctggtgaaacccactcagaccctgactctgacctgcacattctctggcttttccctctctactgccggaatgtcagtgggatggatccgccagcctcctggcaaagctctggagtggctcgctgatatttggtgggacgataaaaagcattataatccatctctgaaggaccgcctcaccatcagcaaggacactagcaagaatcaggtggttctcaaggtgaccaatatggacccagctgataccgctacctactactgtgccagggacatgatcttcaacttctattttgacgtgtggggtcagggcaccaccgtcaccgttagctctgggggagccggtagcggggccgggagcgggagcagcggcgcaggctctggagatatacagatgactcagagcccctctaccctgtctgcttccgtgggcgaccgggtcaccatcacatgctccgcctctagccgcgtcggttatatgcattggtaccagcagaagcccggcaaggcacccaaactcctcatttatgacacctccaagctggcctctggagttccctctcggttttccggaagcggtagcggcaccgagttcacactgaccatctcctctctccagccagatgatttcgccacatattattgcttccagggcagcgggtatccttttacatttggtgggggaactaaagtggagatcaaagggccc

SEQ ID NO: 352 - DNA, 757 bp, Artificial
  Primer
ggcccagccggccaggcgcgaggtgcaactccagcagtctggtcccgagctggagaagcccggcgccagcgtgaagctgtcatgtaaagccagcgggtactcattcactggctataatatgaactgggtgaaacagtcacatggtaagagcctggaatggatcggccatattgacccctattacggtgacacttcttataaccaaaaattcaggggtaaggccaccctgaccgtggacaaatctagcagcacagcctatatgcagctcaaatccctgacatcagaagacagcgctgtttattattgtgtgaaaggcgggtactacggtcattggtatttcgacgtgtggggcgccgggaccactgtgactgtgtcctctggcggatctggcggctctggcggggcctccggagccggatctgggggcggcgacattcagatgacacaatcaccatcttctctgtccgcttccctgggtgagcgcgtctccctcacatgccgggcttctcaggacataggcagctccctcaactggctgcaacagggtccagacggtactatcaagcggctcatttatgctacctctagcctggattcaggcgtgcccaaaaggttttctggatctcggtccggctcagactattccctcactatttcttctctcgaaagcgaggatttcgtggactattactgtctgcagtacgtgagctcacctcctactttcggggcaggcaccaaactcgaactgaaggggccc

SEQ ID NO: 353 - DNA, 760 bp, Artificial
  Primer
ggcccagccggccaggcgcgaagttcagctggtccagtcaggaggaggggtcgaacggcccggcggatctctgcggctgtcctgcgccgccagcggcttcacattcgatgattacggtatgagctgggttagacaagctccagggaaaggactggagtgggtgtccggcatcaattggaacggtggcagcacaggctatgctgatagcgtcaagggcagagttacaatcagcagagacaatgccaagaactctctgtatctccagatgaactccctgagggctgaagataccgcagtctattattgcgccaaaattctgggagccggaagaggatggtactttgatctctgggggaaaggaactacagtcacagtgtctgggggcagcgcaggcagcggctccagcggcggggcttccggatcaggagggtcctccgagctcactcaggacccagctgtgtctgtcgccctcgggcagactgtgcggatcacttgtcagggagattccctccgctcctattatgcctcctggtaccagcagaaacctggccaggcccccgtgctggtcatctacggcaaaaataatcgcccatcaggcattcccgaccggtttagcggatcttcttccgggaatactgcctctctgacaattactggtgcccaagctgaggatgaggccgattactactgtaacagccgcgacagctcaggaaaccacgtggtgttcgggggcggaactaagctcaccgtgctggggccc

SEQ ID NO: 354 - DNA, 778 bp, Artificial
  Primer
ggcccagccggccaggcgccaggtgcagctgcaacaatccggccccgaggttgtgaaaccaggcgcctctgtgaagatgtcttgcaaggcctcaggctatacattcaccagctatgtgattcactgggtgcgccagaaaccaggacagggtctcgattggattggctatattaacccttacaatgatggtacagactatgacgagaagtttaaaggcaaggccacactgacaagcgatacctctactagcaccgcctatatggagctcagctccctccggtcagaagacaccgctgtgtattattgtgccagagaaaaagataattatgctacaggcgcttggttcgcctactggggacaggggactctcgtgactgtgtcaagcggtggagccgggtccggcgccggctctggttccagcggggccggttccggggacattgtgatgacccagtctccagatagcctggctgtgtctctgggcgagagggtgacaatgaattgtaagtcctcacaaagcctcctgtattctaccaatcagaagaactacctggcttggtatcaacagaagccaggccaatctcccaagctcctcatttattgggcttccacaagggagtccggcgtgccagaccggtttagcggatccggctccggcactgatttcaccctcaccatcagctccgttcaagccgaagatgtggccgtctactactgccagcaatattattcctatcgcacctttggcggagggactaaactggagattaaggggccc

SEQ ID NO: 355 - DNA, 772 bp, Artificial
  Primer
ggcccagccggccaggcgcgagatccaactccagcagtctggacctgagctggtgaagccaggtgcctctgtgaaggtgtcatgcaaagcttccggctatgcatttacatcttacaatatgtattgggtgaagcaatcacatggcaagagcctggagtggattggctatattgatccatataatggcgtgacctcttacaaccagaaattcaaggggaaggctaccctcacagttgacaagtcttcttctactgcctatatgcacctcaattcactgacatctgaggactctgccgtgtattattgcgctaggggtggaggaagcatctactatgccatggactattggggacaagggaccagcgtgactgtctcaagcggcggctctggcggcagcggcggcgccagcggcgcaggctccggggggggagatattgtgatgacacaggccgcaccttccgtgcctgtgacccctggggagtcagtgagcatcagctgccgctcctccaagtccctgctgcattccaatggcaatacctatctctattggttcctccagagaccaggacaatccccacagctgctgatctacagaatgtccaacctcgcatctggagtccctgaccggttctcaggcagcggtagcggcaccgcatttactctgcggatttctagggtggaggccgaagatgtgggtgtgtactactgtatgcaacacctggagtatcccctgacttttggagccggaaccaagctcgaactgaaggggccc

SEQ ID NO: 356 - DNA, 757 bp, Artificial
  Primer
ggcccagccggccaggcgccaggtgcaactcgtggaatctggaggcggcgtcgtgcagcccgggaggtctctgcggctgtcatgtgcagcttcaggcttcactttcagcgtctatggtatgaactgggtgagacaggcacctggaaaaggactcgaatgggtggccatcatctggtacgacggcgacaaccaatactacgccgactccgtcaaggggagattcacaatttcacgcgataactccaaaaatacactgtacctccagatgaacggcctgagagctgaggacacagccgtttattactgtgccagggacctccggaccggacccttcgactattggggacaggggacactggtcacagtgtcaagcgcttccggagggtctgcagggtccggatccagcgggggggcttcagggagcggaggggagatcgttctgactcagtctccagactttcagtctgtcacaccaaaggaaaaggtcaccatcacttgccgggcctcacaatccatcggttctagcctgcactggtatcagcagaaaccagaccagtcccccaagctgctcatcaagtacgcttcacagtctttcagcggcgtcccatccaggttctccggctccggttccggcacagacttcactctgaccatcaatagcctcgaagctgaagacgctgctgcttattactgtcaccaaagcagctctctgccctttacttttggtcctggcacaaaggtggacattaaggggccc

SEQ ID NO: 357 - DNA, 748 bp, Artificial
  Primer
ggcccagccggccaggcgccaggtgcagctggtggaaagcggtggcggtgtcgtgcagcccggccgcagcctgagactctcctgcgctgcatcaggttttacattttctagctacgatatgtcttgggtccggcaggcaccaggaaaggggctggagtgggtggctaaagtttcttccggaggggggagcacctactatctcgacactgttcagggccggttcactatatcccgggacaattctaagaatacactgtacctgcagatgaattctctgagggcagaagataccgctgtgtactattgtgcacggcatctgcacggatccttcgcttcctggggacagggcactactgtcaccgtttctagcggcggtgctggatctggagctggatcagggtcctctggagctggctcaggtgagatcgtgctgacccaaagccctgctaccctgagcctctccccaggagagcgggcaacactgtcttgtcaggcatctcaatcaattagcaacttcctgcattggtaccaacagcggccaggccaagcccctaggctgctcattagatacaggtcccaatcaattagcggaataccagccaggttttccggctctggatccggtaccgacttcaccctcaccatctcttccctggaacccgaagacttcgccgtgtattactgtcagcagtctgggtcttggcctctgacattcggaggtggaactaaagtggaaatcaaagggccc

SEQ ID NO: 358 - DNA, 763 bp, Artificial
  Primer
ggcccagccggccaggcgcgaagtgcagctgctggaaagcggcggcgggctggtccagcccggcggatccctgagactgtcatgtgccgccagcggtttcacttttagctcatttccaatggcctgggttcggcaggcaccaggaaaaggcctcgaatgggtgtccacaatatcaacttctggcggtagaacatactatagggactccgtgaagggcagatttaccatttcccgggataatagcaagaatacactgtatctgcagatgaattcactgagggctgaagatacagccgtgtattattgcgccaaatttcgccagtattctggcggctttgactactggggacagggcactctcgtcacagtgagctctggcgggtccggaggctctggcggcgcctcaggcgcaggctccggaggcggcgacattcagctcactcaacccaacagcgtgtcaacttctctgggatccaccgtgaagctgtcctgtactctcagctctgggaatatcgaaaataactacgtgcattggtaccagctctatgaggggcggagccccactaccatgatttatgacgacgataaacgccctgacggtgtgcctgatagattttctggcagcatcgatcggtctagcaatagcgcattcctgactatccataatgtggcaatcgaggatgaggctatctacttctgtcactcctatgtgagctccttcaacgtcttcggtggcggcacaaaactgactgttctcgggccc

SEQ ID NO: 359 - DNA, 760 bp, Artificial
  Primer
ggcccagccggccaggcgcgaagaacaggttgttgagtcagggggcggatttgtgcagcctggaggatctctgagactcagctgcgcagccagcggcttcaccttttcaccatactggatgcactgggtgagacaagctcctggcaagggactcgtctgggtgtcacggattaattctgacggatcaacatactacgcagactcagtcaaaggaaggtttaccatatccagagataacgctagaaacacactgtatctgcagatgaactcactcagagctgaggatacagcagtttactactgtgcaagagaccggtattatggtcctgagatgtggggccagggcacaatggtgaccgttagctctggcggcgcaggctctggggctggatcaggaagctccggtgctggtagcggcgatgtggtgatgacccagtctccactcagcctccccgttacactcgggcaacccgcctctatttcttgccgctcctcccaatccctcgtgtactctgacggcaatacatacctgaattggttccagcagagacctgggcagtcaccaaggagactcatttacaaggtgagcaatcgcgacagcggggtgcccgaccggttcagcggcagcggctcagggaccgattttaccctcaagatttcaagggtggaagctgaagatgtgggagtctattattgtatgcagggcacccactggcccctgacatttggcggcgggacaaaggtcgagatcaaggggccc

SEQ ID NO: 360 - DNA, 760 bp, Artificial
  Primer
ggcccagccggccaggcgccaggtcgagctggtggagtctggcggggggctggtgcaacctgggggaagcctgaggctgtcctgcgctgcatcagggttcacattctctagctatgcaatgtcctgggtgaggcaggcccctggaaaaggactggagtgggtctctgcaatcaatgcctctggcacccgcacttattatgctgacagcgtcaaggggaggtttactatttctagggataactctaaaaataccctgtacctccagatgaactcactcagggccgaggatactgcagtttactattgcgctaggggtaaaggtaacacccacaagccttacggatatgtgaggtacttcgacgtgtgggggcagggaaccggtggctccggcggaagcgggggagcttccggggctggctctggtgggggcgacatcgtgctcacccagtccccagccactctgagcctgagccctggagaaagagcaacactgtcttgccgggcctcccagtccgtttccagcagctacctggcctggtatcagcagaaaccaggccaggcaccaaggctcctgatctatggtgcctcttccagagcaaccggcgtgcctgctcggttctccgggtccggctcagggaccgacttcacactgactatatcctccctggagccagaggactttgccacatactattgtctgcaaatctacaatatgcccattacctttggccagggtaccaaagtcgagatcaaggggccc

SEQ ID NO: 361 - DNA, 772 bp, Artificial
  Primer
ggcccagccggccaggcgccaggtccagctgcagcagtctggatccgagctcaaaaagcccggagccagcgttaaggtttcctgcaaagcctctggctataccttcactaattacggtgtgaactggattaagcaggccccaggccaggggctccaatggatgggctggataaaccctaatactggagagcctactttcgacgatgatttcaaggggcgcttcgccttctctctggatacctccgtgtcaactgcctacctccagatctcaagcctgaaagccgacgatactgccgtgtacttctgttctaggtccagagggaagaacgaggcctggttcgcatactggggtcaggggacactggtgactgtgagctctggaggatcagcagggtcagggtcttccggcggggctagcggctcagggggcgacattcagctcacccaatcaccactgtctctgcccgtgaccctcggacagcccgcttcaatctcatgccggtcttctcagtcactcgtccatcggaacggcaacacttatctgcactggtttcaacagcggccaggccaatctccccgcctgctgatttacactgtgagcaatcggttctcaggtgttcctgacagatttagcgggagcggtagcggcactgattttactctgaagatttcccgcgtcgaagccgaggacgtcggggtgtacttttgcagccagagctctcatgtgccccccaccttcggcgcagggacacgcctggaaattaaggggccc

SEQ ID NO: 362 - DNA, 757 bp, Artificial
  Primer
ggcccagccggccaggcgccaggtgcagctgcagcaatctggcgccgaagtgaaaaaaccaggttcctccgtcaaggtgagctgcaaggcctccggctacacctttacctcatacaacatgcactgggtgaaacaagctcctggtcagggcctggagtggattggcgcaatctatcccgggaatggcgacacttcttataaccaaaagttcaaaggaaaggccacactcacagccgacgaaagcaccaatactgcctacatggagctgtctagcctccgctctgaggatactgccttctactactgtgctcggtccacttactacgggggggattggtacttcgatgtgtgggggcaaggcactactgtcacagtttcttctgggggggccgggagcggggccggaagcggcagctccggcgcaggctccggggatatccagctgacacagagcccttcatcactctccgcctctgttggagatagagtcacaatgacttgtagggcctcctcttccgtgtcatacatccactggttccagcagaagcccggtaaggctcccaagccttggatttatgccacatccaatctggcctcaggtgtgcccgtccgcttctccggtagcggatctgggactgattatactttcacaattagctctctgcagccagaagatattgcaacttactattgccaacagtggacatccaatcctcctacttttggaggggggactaagctcgaaataaaggggccc

SEQ ID NO: 363 - DNA, 760 bp, Artificial
  Primer
ggcccagccggccaggcgccaggttcagctccaagagtcaggtcctgggctggttaagccttctgagacactgagcctgacctgcaccgttagcggcttctccctgatcggctacgatctgaactggattcggcagccacccggaaagggcctggaatggattggcataatctggggagacgggacaactgactataattctgccgttaagtcacgcgtgaccatatctaaagacacaagcaagaaccagttcagcctgaaactgtcctcagtcacagcagcagatactgctgtgtattactgtgcccgcgggggctattggtacgctacctcatattactttgattactgggggcagggcaccctggtgaccgtctcctctggaggctctggtgggtctggaggagcatctggggccgggagcggcgggggggatattcagatgactcaatcaccctcaagcctctcagcctcagtcggggaccgggtgacaatcacctgtagggcttcacaaagcatatccaacaatctgaattggtaccagcaaaaaccaggaaaagccccaaaactcctgatatactatacctcccggttccacagcggggtgcctagcaggttcagcggctccggcagcggcactgatttcactttcaccatttcctccctgcaaccagaggacattgcaacttattattgccagcaggagcataccctgccatatactttcggccagggtacaaagctggagataaaggggccc

SEQ ID NO: 364 - DNA, 763 bp, Artificial
  Primer
ggcccagccggccaggcgcgaagtgcaactggtcgaaagcgggggtggactggtgcagcctgggggcagcctgcgcctgagctgtgcagcttcaggctttaccttcatcagctacgctatgtcttgggtgagacaggcccccggaaaaggactcgaatgggtggctagcatctcaagcggtggcaatacatactaccccgacagcgtcaagggccggtttaccatctcacgcgacaatgccaagaattccctgtacctgcagatgaactccctgcgcgctgaagatacagccgtctattattgcgctcggctggacggctactactttggcttcgcatactggggccaggggaccctggtgacagtcagctccggggggagcgccggctcagggtcctccggtggtgcctctggctcagggggggacattcaaatgacacagagcccctcttctctctcagctagcgtgggcgaccgcgttacaattacttgcaaagccagcgaatccgtcgataactatgggaagtccctgatgcactggtatcaacagaaacctggaaaggctcccaaactgctcatctaccgggcttcaaacctggagagcggtgtgccctcacggttctccggatctggaagcgggactgactttaccctcaccatctcctcactccaaccagaggatttcgctacatattattgccagcaatctaacgaggatccatggacattcggggggggcacaaaggttgaaatcaaggggccc

SEQ ID NO: 365 - DNA, 754 bp, Artificial
  Primer
ggcccagccggccaggcgcgaggtgcaactcgtccagagcggcgccgaggttaagaagcctggcgagtccctgaaaatttcctgcaaaggcagcgggtactctttcactacatactggctgggttgggtgcggcagatgcccgggaaggggctggattggatcggcataatgtccccagtggattcagacatacgctatagcccctccttccagggtcaggtgaccatgagcgtcgataagagcattactaccgcctacctccagtggaattccctgaaggcctctgatacagccatgtactactgcgcccgcagacgcccaggacagggatacttcgacttctggggccagggaaccctcgtgaccgtttcaagcggcggggcagggtctggcgcaggaagcggcagcagcggagccggatctggggatattcagatgacccagtctccttcttccctctctgctagcgtcggcgatagggttacaatcacttgcagggccagccagggcatatcatcttggctggcttggtatcagcagaagccagaaaaggcccctaagagcctcatatatgctgccagctccctgcagtccggcgtgccctcccgcttctcaggctcaggttcagggacagacttcacactgacaatctcctccctccagccagaggatttcgccacctattattgccaacagtacaatatctacccttacacctttggccagggcaccaaactggaaatcaaggggccc

SEQ ID NO: 366 - DNA, 754 bp, Artificial
  Primer
ggcccagccggccaggcgcgaagtgcaactggtggagtctgggggaggcctggttcagcccggtgggagcctgcggctgtcctgcgccgcttccggctactcattcaccggatactacatccattgggtgaggcaggcccctgggaagggcctggaatgggtggctagagtcattcctaatgccggtggaacaagctacaatcagaaattcaaggggcggtttaccctgagcgttgacaactctaagaatactgcatatctgcagatgaactctctgcgggccgaggacaccgccgtgtattactgcgccagggaaggaatctattggtggggccaaggtaccctggtgacagtctcttccgggggctcaggaggatctggaggtgcatccggcgccggaagcggagggggcgacatccagatgacacagtccccttcttctctctctgcatccgttggagatagagttacaattacttgtcggagctctcagtcactggtgcacagcaacggtaacacattcctgcactggtaccagcagaaacctggcaaagcccctaagctgctgatatacacagtctccaaccggttctctggagtgccctccaggttttcaggaagcgggtcagggacagactttaccctgactatctcctctctgcaacctgaggatttcgccacctatttctgcagccaaactacccatgttccctggacttttggtcaggggaccaaggttgagatcaaggggccc

SEQ ID NO: 367 - DNA, 748 bp, Artificial
  Primer
ggcccagccggccaggcgcgaagtccaactcgtggagtccgggggaggcctggtgcagcccggtgggagcctgaggctctcctgtgccgccagcggcttcacattctcttcctacggtatgtcatgggtcaggcaggcccccggaaaaggcctggaatgggtcgcaaccataacatccggcggcagctatacatactacgtggatagcgttaaggggaggttcacaatttcccgggacaacgccaaaaacacactgtacctgcagatgaactctctgcgggccgaggataccgctgtgtactattgcgtgaggataggcgaagatgctctggactactggggacaggggactctggtcacagtgtcaagcggcggcagcgccggctcaggtagctctgggggtgcctctggatccggcggcgatatccagatgacacaatctccttccagcctgtccgcctccgtgggtgacagggtgaccattacatgtagagcatcacaggacatcgcagggtccctgaattggctgcaacaaaagcctgggaaagctatcaaaaggctgatttacgcaacaagctctctcgacagcggcgttcctaagagattctctggctctaggtcaggaagcgattataccctgactatctctagcctccagcctgaagattttgccacttattattgcctccagtacgggtctttcccacctacctttggtcagggcacaaaagtcgagataaaagggccc

SEQ ID NO: 368 - DNA, 751 bp, Artificial
  Primer
ggcccagccggccaggcgcgaagttcagctggtggagtccggggggggtctggtccagccaggaggttcactccgcctctcttgcgcagcctcaggcttcacctttagctcttacgtgatgtcctgggtcaggcaggcccctggcaagggtctcgaatgggttgccacaatctcttcaggcggaagctacacctactatcccgactctgttaaaggaagattcacaatttccagagataacgccaaaaacacactgtacctgcaaatgaattcactgagagctgaggatactgctgtgtactactgcgccagacgcggtgactccatgatcaccaccgactattggggtcaggggactctggtcaccgtgtcatccgggggagccgggagcggggctggcagcggatcttctggagcaggttctggcgacatccagatgacacaaagcccttcatccctctctgcatctgtcggcgatcgcgtgactataacctgcaaagcctcccaggacgttggaactgccgttgcttggtaccagcagaaacccggcaaggcacctaagctgctgatctactgggctagcacaaggcatactggggtgcccagccgcttctccggttccggcagcggtacagatttcacactcactattagctctctgcagcctgaagacttcgccacctactattgccagcagtactctagctaccggaccttcggacagggaacaaaagtggagatcaaggggccc

SEQ ID NO: 369 - DNA, 760 bp, Artificial
  Primer
ggcccagccggccaggcgccaggtgcagctgcagcagtccggcgccgagctggtgaagccaggtgcatctgttaagctgtcctgcaaggcatccggctatactttcacctcctacgatatcaactgggttcggcagaggcctgagcaaggactggagtggattgggtggatcttccccggagatggatctaccaagtataacgagaagttcaaggggaaagccaccctgaccacagataaaagctcaagcaccgcctatatgcagctctctcggctgacatctgaagattctgccgtctatttttgcgctcgggaggactactacgacaactcatattattttgactactggggtcaggggacaacactcactgtctccagcggcggctcaggtgggagcggcggggcttctggtgccggatccggaggcggtgatatccagatgacccagacaacttcaagcctgtccgcctcactgggggatcgggtcaccatttcttgcagagcctctcaggatatcagcaattacctgaattggtaccagcaaaaacccgatggaacagtgaaactgctgatctactacacatctcggctgcatagcggagtgccctccaggttcagcggctccgggtctggcacagactacagcctgaccatcagcaacctggaacaggaggacattgccacctatttttgtcaacaaggaaataccctcccttggacatttgggggaggcaccaagctggaaattaaggggccc

SEQ ID NO: 370 - DNA, 766 bp, Artificial
  Primer
ggcccagccggccaggcgccaggtgcaactccaggaatccggtcccggcctggtgaagccatctcagacactgtccctgacctgcacagtttccggcggcagcatctctagcggagactatttctggtcctggatcagacagctcccaggcaagggcctggagtggatagggcatattcataactctggaacaacctactataatccctctctcaaatcacgggttactatctccgtggacacttccaagaaacagttctccctcagactgtcctcagttaccgcagccgacaccgctgtgtattactgcgcaagggacagggggggcgactattactacggcatggacgtgtggggccaaggtacaactgttaccgtttcctcaggtggatcagccggcagcggatcttctggtggcgcctccggatctggcggagaaattgtgctcactcaatccccagggacactgtccctcagccctggcgaacgggccactctgtcctgcagggctagccagggcattagccggagctacctggcctggtatcagcaaaagcctgggcaggccccctctctgctgatctatggtgcatcctcccgcgccaccgggatccctgacagattttccggatccggtagcggtacagacttcactctgacaatttcccgcctggagcccgaggattttgctgtgtattactgccagcaatttggttcttcaccatggacctttggtcaagggacaaaggtggaaataaaggggccc

SEQ ID NO: 371 - DNA, 168 bp, Artificial
  Synthesized sequence
ccctttaatcagatgcgtcgtgcaaatgtgaggtagcaacgcagtgagcagactccctcacaccttcgggcaagggacaaaggtcgaaattaaagggcccgaggcccactcgtatgattattcactgcaacgcaagcgaaaactacaaggtcgcccttattactacca

SEQ ID NO: 372 - DNA, 189 bp, Artificial
  Synthesized sequence
ccctttaatcagatgcgtcgaaagtcaaagtgcgtttcgtgcagtgttaagtgcacatttcgtttcgaggcccagccggccaggcgccaggtgcagctggtccaatctggtgcagaagtgaagaaacctggagcttccgtgaacactgcggctatgagagagcaacacaggtcgcccttattactacca

SEQ ID NO: 373 - DNA, 184 bp, Artificial
  Synthesized sequence
ccctttaatcagatgcgtcgaaagtcaaagtgcgtttcgtgcagtgagaaacctggagcttccgtgaaggtgagctgtaaggggtctgggtatacctttacaagctattggatgcattgggtgagacaagcccccggccactgcggctatgagagagcaacacaggtcgcccttattactacca

SEQ ID NO: 374 - DNA, 191 bp, Artificial
  Synthesized sequence
ccctttaatcagatgcgtcgaaagtcaaagtgcgtttcgtgcagtgggtgagacaagcccccggccagcgcctcgaatggatcggggaaattgacccttctgaatctaacactaactacaatcagaaatttaaggggagagtgaccactgcggctatgagagagcaacacaggtcgcccttattactacca

SEQ ID NO: 375 - DNA, 189 bp, Artificial
  Synthesized sequence
ccctttaatcagatgcgtcgaaagtcaaagtgcgtttcgtgcagtgaatcagaaatttaaggggagagtgaccctgaccgtggacatttcagcttctactgcctacatggaactgtccagcctgcgctctgaggacacagccgcactgcggctatgagagagcaacacaggtcgcccttattactacca

SEQ ID NO: 376 - DNA, 189 bp, Artificial
  Synthesized sequence
ccctttaatcagatgcgtcgaaagtcaaagtgcgtttcgtgcagtgtgcgctctgaggacacagccgtttactattgtgcccggggcgggtacgacggttgggactatgccattgactactgggggcaaggaaccctggttaccactgcggctatgagagagcaacacaggtcgcccttattactacca

SEQ ID NO: 377 - DNA, 189 bp, Artificial
  Synthesized sequence
ccctttaatcagatgcgtcgaaagtcaaagtgcgtttcgtgcagtgggggcaaggaaccctggttacagtctcaagcggtggaagcgccggttcaggttcctcaggaggggcctcagggtcaggcggagatgtcgtgatgacccactgcggctatgagagagcaacacaggtcgcccttattactacca

SEQ ID NO: 378 - DNA, 189 bp, Artificial
  Synthesized sequence
ccctttaatcagatgcgtcgaaagtcaaagtgcgtttcgtgcagtgaggcggagatgtcgtgatgacccaatctccactgagcctgcctgttactcccggcgagcccgcatcaatcagctgcagatcctctcaatccctggctcactgcggctatgagagagcaacacaggtcgcccttattactacca

SEQ ID NO: 379 - DNA, 189 bp, Artificial
  Synthesized sequence
ccctttaatcagatgcgtcgaaagtcaaagtgcgtttcgtgcagtgtgcagatcctctcaatccctggctaagagctatggaaatacctacctgtcatggtacctccagaagcctggccaatcaccccagctgctgatctacgcactgcggctatgagagagcaacacaggtcgcccttattactacca

SEQ ID NO: 380 - DNA, 772 bp, Artificial
  Primer
ggcccagccggccaggcgccaggtccagctggttcaaagcggagccgaggttaaaaaacctggttctagcgtgaaagtgagctgcaaggcctctggctacgcattctcttacagctggatcaattgggtgcgccaggccccaggtcagggtctggagtggatgggcaggatctttccaggagacggagataccgattacaacggcaagtttaaagggagggtgactataaccgctgacaagagcacttcaacagcctatatggaactcagctctctcagaagcgaggatacagcagtctactattgtgctcggaatgtctttgacgggtactggctggtgtactggggccagggaaccctggtcacagttagcagcgcaggtggggccggctctggggcagggagcggctcctctggcgccggcagcggggacatagtgatgacacaaactcctctgtctctgccagttacccccggagaacccgccagcatttcttgtagatcctctaaaagcctgctgcatagcaatgggatcacctacctgtactggtatctgcagaaacccggccaatcccctcagctgctgatttaccaaatgtccaacctggtgtcaggagtcccagatcggttcagcggatccggaagcggtactgattttaccctcaaaatatcaagggtggaagccgaggacgtgggcgtgtactattgcgcccagaatctggaactcccttatacattcggaggcggcacaaaagtggaaataaaagggccc

SEQ ID NO: 381 - DNA, 189 bp, Artificial
  Synthesized sequence
ccctttaatcagatgcgtcgaaagtcaaagtgcgtttcgtgcagtgtcaccccagctgctgatctacggcatttcaaacagattcagcggcgtgcctgatcgcttctccggttcagggtctggtactgatttcacactgaagacactgcggctatgagagagcaacacaggtcgcccttattactacca

SEQ ID NO: 382 - DNA, 190 bp, Artificial
  Synthesized sequence
ccctttaatcagatgcgtcgaaagtcaaagtgcgtttcgtgcagtgtctggtactgatttcacactgaagatctctcgggtggaggcagaggatgtgggcgtctactactgtctccagggtacacaccagccatatactttcggcactgcggctatgagagagcaacacaggtcgcccttattactacca

SEQ ID NO: 383 - DNA, 170 bp, Artificial
  Synthesized sequence
ccctttaatcagatgcgtcgaaagtcaaagtgcgtttcgtgcagtggtacacaccagccatatactttcgggcaagggacaaaggtcgagatcaaggggcccaccggtcaattctaccaactttcactgcggctatgagagagcaacacaggtcgcccttattactacca

SEQ ID NO: 384 - DNA, 24 bp, Artificial
  Primer
ataaaaataggcgtatcacgaggc

SEQ ID NO: 385 - DNA, 21 bp, Artificial
  Primer
cggcggatttgtcctactcag

SEQ ID NO: 386 - DNA, 21 bp, Artificial
  Primer
cgcaaatgggcggtaggcgtg

SEQ ID NO: 387 - DNA, 18 bp, Artificial
  Primer
tagaaggcacagtcgagg

SEQ ID NO: 388 - DNA, 35 bp, Artificial
  Primer
cagtggagagggtgaaggtgatgcaacatacggaa

SEQ ID NO: 389 - DNA, 20 bp, Artificial
  Primer
tttgcttcagtcagattcgc

SEQ ID NO: 390 - DNA, 760 bp, Artificial
  Primer
ggcccagccggccaggcgcgaggtgcagctggtgcaaagcgggccaggcctcgtccagcctgggggatctgttagaatctcatgtgctgcctcaggatatacttttacaaactatggaatgaattgggtgaagcaggcacctgggaagggcctggagtggatgggttggattaacacttatacaggcgaatcaacatatgccgactcctttaagggccggttcaccttttctctcgacacttccgccagcgccgcctacctgcaaatcaacagcctgagggccgaagatactgccgtgtattattgcgcaagatttgctattaagggggactactggggtcaagggaccctgctgacagtgtccagcggcgggagcggcggttccggcggagcttccggagccgggtccggcggaggggatattcagatgacccagtcacccagcagcctctctgcatctgtgggggacagggtgaccatcacctgtagatcaacaaaatctctgctgcatagcaacggaatcacttacctgtactggtatcagcagaagcctggcaaagccccaaaactgctgatctatcagatgtccaatctcgcatctggcgtcccatctaggtttagctcctccggctccggtacagacttcaccctgaccatatcaagcctgcagccagaggactttgccacttactattgcgctcagaatctcgaaatccctaggacatttggacagggcacaaaggtcgaactgaaagggccc

SEQ ID NO: 391 - DNA, 760 bp, Artificial
  Primer
ggcccagccggccaggcgcgaggttcaactcgtccaatctggccctgggctcgtccagcccgggggatccgtccgcatctcctgcgccgcctctggctataccttcactaattatggcatgaactgggttaaacaggccccaggcaaaggtctggagtggatgggctggattaatacctataccggcgagtccacatacgccgatagctttaaggggaggttcactttcagcctcgataccagcgcttcagcagcatacctgcagattaactctctgcgcgccgaagataccgctgtctactattgcgcccggttcgctattaagggggattactgggggcagggcacactcctgaccgtttcaagcggcgggtccgccggctccggctcatctggcggggcatctgggagcggaggggacatacaaatgacacagtctccaagctctctgagcgcttctgtgggggatcgcgtcaccattacatgcagatccacaaaatccctgctgcatagcaatggcattacttatctgtattggtaccagcagaaacctggcaaagctcccaaactgctgatataccagatgtccaatctggcctccggtgttcccagcagattctcaagctccggcagcgggacagactttactctgaccatcagcagcctgcagcccgaggatttcgccacttactactgcgctcagaacctggaaatcccaagaacatttggccagggcactaaggttgaactgaaggggccc

SEQ ID NO: 392 - DNA, 751 bp, Artificial
  Primer
ggcccagccggccaggcgcgaggtgcagctggttgagtctggtgggaaactgctcaagcccggaggctcactgaagctgtcttgtgctgcttctggctttaccttcagcagcttcgcaatgtcttggtttcggcaaagcccagagaagcgcctggagtgggttgccgagatatcttctggagggtcatacacctactaccccgacactgttacaggtcggttcaccatctccagggataatgccaagaataccctgtatctggagatgtcttctctcaggtcagaagataccgctatgtactattgcgctagaggtctctggggttattatgcactcgattactggggccagggtactagcgtcacagtgtcctctggtggggccggctctggagccgggagcgggtcaagcggagccggatctggccagattgtcctcatccagtcccccgccatcatgtctgcttctccaggagagaaggtcaccatgacatgttccgcatcatcctccgtttcttacatgtattggtatcagcagaagccaggctctagcccacgcctgctgatctatgacacttctaacctcgcctccggagtgcccgtgcgcttttccggctcaggcagcggaacatcatatagcctgaccataagccgcatggaagccgaggatgccgcaacctattattgtcaacagtggtcagggtatccctacacattcgggggaggcaccaaactggaaattaaggggccc

SEQ ID NO: 393 - DNA, 793 bp, Artificial
  Primer
ggcccagccggccaggcgccaagtgcagctggttcagtccggggccgaagtcaagaagcctgggtctagcgtgaaggtctcttgcaaagccagcgggggaactttcaaccggtatactgttaactgggtgcggcaagctcctggccagggactggagtggatggggggaatcatccccatatttggaaccgctaactatgcacagcgcttccagggcagactgactataaccgcagatgagtccacctcaaccgcctacatggagctgtcctctctgcggtccgacgatacagccgtgtacttttgcgcccgggagaacctggacaactctggcacttactattacttcagcggctggttcgacccttggggacaaggcaccagcgtcacagtctcatctggcggttctggggggagcggcggcgcttctggggccggaagcggtggcggtcagagcgcactgacccagcctcgcagcgtctccggctcccctgggcagagcgtgacaatatcttgtacaggcacctcctccgatatcggggggtataatttcgtgtcatggtaccagcaacatcccggcaaagccccaaagctgatgatctacgacgccactaagaggccttccggggtgcccgataggttcagcgggagcaaatctggtaatactgcctcactgactatatcaggcctgcaggcagaagacgaggcagattattactgctgttcttacgccggtgactacacacctggtgtggtgtttgggggcggcaccaagctgactgtgctggggccc

SEQ ID NO: 394 - DNA, 757 bp, Artificial
  Primer
ggcccagccggccaggcgccaggtccagctggtcgagtctggcggaggcgccgtgcagcccgggaggtccctgagactgtcttgcgctgcttcaggtttcactttttcttcctacggcatgcactgggtccgccaagctcctggaaagggactggaatgggtcgccgtcatactgtacgacgggagcgacaagttttatgccgattcagtgaagggtcggtttactatttcacgcgataattccaagaacacactgtatctgcagatgaattccctgcgggctgaagatacagccgtgtactactgtgcaaaagtggccgtggcagggactcactttgactattggggccaggggactctggtgactgtgtcctctgcaggcggttccgccggctctggctccagcgggggcgcttcaggctccgggggcgatatccaaatgacccaaagcccatcctcactctccgcctctgttggcgatagagtcactattacctgcagggcctctcaggggatccgcaatgatctcggatggtaccagcagaaacccggaaaagctccaaaactgctgatatacgcagcttcttctctgcagtccggggtcccctcccggttctccggtagcggttctggaaccgactttacactgactatatcctctctccagcctgaagacttcgctacatattactgccagcagctgaacagctaccctcccacattcggcggcggtactaaggtggaaatcaaagggccc

SEQ ID NO: 395 - DNA, 763 bp, Artificial
  Primer
ggcccagccggccaggcgcgaagttcagctcgtggagtctggcggaggcgtggtccaacctggcaggtccctgaggctgtcttgttctgccagcggatttacattttccgggtacggactgtcctgggtcagacaggctccagggaaaggcctcgaatgggtggcaatgatctctagcggaggctcatacacctattacgccgactccgtcaaggggcgcttcgccatcagcagagataatgcaaagaatactctcttcctccagatggattctctccggcccgaggacaccggtgtgtacttctgtgctcgccatggggatgacccagcctggtttgcttactggggccagggaactcctgtgaccgtttctagcgggggggctggcagcggggccggttcaggttcttccggcgccggctccggggacatccagctcactcagagcccatcttcactgtcagcatccgtcggagatagagtgactataacctgttcagtgtcctcatcaatcagctccaacaatctgcactggtaccagcagaaaccaggaaaggcaccaaaaccctggatatacggcacctcaaatctggcttccggtgtgccttccagattctcagggagcggatccggcaccgactacacctttacaatcagctccctgcagcccgaggacattgcaacatactactgtcaacagtggagctcctatccctatatgtacaccttcggacagggaacaaaggttgagattaaagggccc

SEQ ID NO: 396 - DNA, 754 bp, Artificial
  Primer
ggcccagccggccaggcgcgaggtgcagctcgtcgagtccggaggcggcctggttcagcctggcgggtctctccgcctgtcctgcgccgcctccggattcgactttagcagatactggatgtcctgggtgagacaggctcctggaaaaggactcgaatggatcggggagatcaaccccgattcttccaccatcaactacgcacctagcctgaaagataaattcatcatttccagagacaatgccaaaaattcactgtacctccaaatgaacagcctgagagctgaggatactgctgtctactactgcgctaggcccgatgggaattactggtacttcgatgtgtgggggcagggcactctggttaccgtgtcatcaggtggctccggagggtccggcggcgcaagcggagccggatccggcggaggagacatccagatgacacagtctccatccagcctcagcgcctccgttggcgatcgggtgacaatcacctgcaaggcctcacaggacgtcggaatcgccgttgcttggtatcaacaaaagcccgggaaggtccccaagctgctgatttattgggcctctacacggcacacaggtgttccagatcgcttctctggtagcggctccggaaccgactttactctgactatatcttctctgcagcccgaggatgtggccacttactactgtcagcaatatagctcctacccatacacttttggccaggggacaaaagtggagatcaaagggccc

SEQ ID NO: 397 - DNA, 760 bp, Artificial
  Primer
ggcccagccggccaggcgccaggtgcagctgcaagaatcagggccaggactcgtcaaaccctctcaaacactgtctctgacttgtaccgtgtctgggggctccatctcatccggggattactactggtcatggatcaggcaaccacctggcaaaggtctggagtggattggctatatctactactctgggtcaaccgattataacccaagcctcaagtctcgggttacaatgagcgtggatactagcaagaatcaattctcactcaaggtgaactctgttactgccgctgacaccgccgtgtactattgcgctcgggtctctatcttcggtgtggggacctttgactattggggtcaaggaacactggtcactgtttcaagcggcggctctgcagggtcaggctcatccggaggcgcctccggctctggcggcgaaatagtgatgactcagtcaccagctactctgtccctctcccctggagagagggctacactctcttgccgcgcctcacagtctgtgagcagctacctcgcttggtaccagcagaaaccaggtcaggccccccggctgctgatctatgacgctagcaatcgggctactggcatccccgccagattttctggatctgggtcaggcaccgacttcacactgactataagctcactggagcccgaagacttcgccgtgtattactgccatcagtatggaagcacccccctgacctttgggggtggtaccaaagccgagattaaggggccc

SEQ ID NO: 398 - DNA, 772 bp, Artificial
  Primer
ggcccagccggccaggcgcgaggttcagctcctggagtccgggggcggactggtgcagcccgggggctcactgaggctgagctgcacagcctctggcttcacatttagctcctacgccatgaattgggtgagacaagcccctggaaaggggctggagtgggtgtctgctatttcaggctcaggggggacaaccttttatgccgacagcgtgaagggcaggttcaccatttcacgcgataactcacgcactaccctctatctgcagatgaattccctgcgggcagaagacacagccgtctattattgtgcaaaagacctgggatggtctgactcatattattattattatgggatggatgtttgggggcaggggaccaccgtgaccgtcagcagcggcggggcaggatctggggccgggtctggctcatcaggggccggttctggggatatacagatgacccagttcccatcatctctctcagcctctgtcggggatagggttaccattacttgcagagccagccagggaatcagaaatgatctgggctggtatcaacagaaaccaggtaaagcccccaagaggctcatctacgccgcatcccgcctgcatcggggagtcccttcacgcttttccggctctggctcaggtaccgagttcactctcactatttccagcctccagccagaggattttgcaacctactactgcctgcaacataattcttatccctgttcatttggtcagggcacaaagctcgaaattaaggggccc

SEQ ID NO: 399 - DNA, 751 bp, Artificial
  Primer
ggcccagccggccaggcgcgaagtccaactggttcagtccgggggcggcctggtgaaacccggcggctccctgaggctctcatgcgccgccagcggatttactttttcctcatttgccatgcactgggtgaggcaggcaccaggaaaaggactggagtggatcagcgtcattgatacaagaggtgcaacatattacgctgacagcgtgaaggggagatttacaattagccgcgataacgccaagaactccctgtacctgcagatgaactccctgcgggctgaagacacagccgtgtactattgtgcaaggctgggtaatttttattacggcatggacgtttgggggcaggggactactgtgacagtttcctcaggggggagcggggggagcgggggggctagcggcgctggctccggagggggagagatcgtcctgacacagtcacccgggactctgtctgtgagccctggcgagagagcaactctgtcatgcagggccagccaaagcatcggctcatctctgcactggtaccagcagaaacccggtcaggccccacgcctgctgatcaaatatgccagccagagcctgtcaggcattcctgacagattttctgggagcggatcaggaacagatttcacactcacaatatccaggctggagcccgaagacttcgctgtctactactgccaccagtccagcagactccctcacaccttcgggcaagggacaaaggtcgaaattaaagggccc

SEQ ID NO: 400 - DNA, 775 bp, Artificial
  Primer
ggcccagccggccaggcgccaggtgcagctggtccaatctggtgcagaagtgaagaaacctggagcttccgtgaaggtgagctgtaaggggtctgggtatacctttacaagctattggatgcattgggtgagacaagcccccggccagcgcctcgaatggatcggggaaattgacccttctgaatctaacactaactacaatcagaaatttaaggggagagtgaccctgaccgtggacatttcagcttctactgcctacatggaactgtccagcctgcgctctgaggacacagccgtttactattgtgcccggggcgggtacgacggttgggactatgccattgactactgggggcaaggaaccctggttacagtctcaagcggtggaagcgccggttcaggttcctcaggaggggcctcagggtcaggcggagatgtcgtgatgacccaatctccactgagcctgcctgttactcccggcgagcccgcatcaatcagctgcagatcctctcaatccctggctaagagctatggaaatacctacctgtcatggtacctccagaagcctggccaatcaccccagctgctgatctacggcatttcaaacagattcagcggcgtgcctgatcgcttctccggttcagggtctggtactgatttcacactgaagatctctcgggtggaggcagaggatgtgggcgtctactactgtctccagggtacacaccagccatatactttcgggcaagggacaaaggtcgagatcaaggggccc

SEQ ID NO: 401 - DNA, 192 bp, Artificial
  Synthesized sequence
atatagatgccgtcctagcgaatccttgcgtcaatggttcgcagtgtttttgcttcagtcagattcgcggtaccatggtgagcaagggcgaggaaaccacaatgggcgtaatcaagcccgacatgaagatcaagctgaagatggagcactgccgtgtaaaatccgagaaccctgggcacaggaaagatactt

SEQ ID NO: 402 - DNA, 188 bp, Artificial
  Synthesized sequence
atatagatgccgtcctagcgaatccttgcgtcaatggttcgcagtgcatgaagatcaagctgaagatggagggcaacgtgaatggccacgccttcgtgatcgagggcgagggcgagggcaagccctacgacggcaccaacaccactgccgtgtaaaatccgagaaccctgggcacaggaaagatactt

SEQ ID NO: 403 - DNA, 185 bp, Artificial
  Synthesized sequence
atatagatgccgtcctagcgaatccttgcgtcaatggttcgcagtggccctacgacggcaccaacaccatcaacctggaggtgaaggagggagcccccctgcccttctcctacgacattctgaccaccgcgttcgcctacactgccgtgtaaaatccgagaaccctgggcacaggaaagatactt

SEQ ID NO: 404 - DNA, 187 bp, Artificial
  Synthesized sequence
atatagatgccgtcctagcgaatccttgcgtcaatggttcgcagtggaccaccgcgttcgcctacggcaacagggccttcaccaagtaccccgacgacatccccaactacttcaagcagtccttccccgagggctactcttcactgccgtgtaaaatccgagaaccctgggcacaggaaagatactt

SEQ ID NO: 405 - DNA, 188 bp, Artificial
  Synthesized sequence
atatagatgccgtcctagcgaatccttgcgtcaatggttcgcagtgcttccccgagggctactcttgggagcgcaccatgaccttcgaggacaagggcatcgtgaaggtgaagtccgacatctccatggaggaggactccttcactgccgtgtaaaatccgagaaccctgggcacaggaaagatactt

SEQ ID NO: 406 - DNA, 190 bp, Artificial
  Synthesized sequence
atatagatgccgtcctagcgaatccttgcgtcaatggttcgcagtgctccatggaggaggactccttcatctacgagatacacctcaagggcgagaacttcccccccaacggccccgtgatgcagaaaaagaccaccggctgggcactgccgtgtaaaatccgagaaccctgggcacaggaaagatactt

SEQ ID NO: 407 - DNA, 191 bp, Artificial
  Synthesized sequence
atatagatgccgtcctagcgaatccttgcgtcaatggttcgcagtggcagaaaaagaccaccggctgggacgcctccaccgagaggatgtacgtgcgcgacggcgtgctgaagggcgacgtcaagcacaagctgctgctggagggcactgccgtgtaaaatccgagaaccctgggcacaggaaagatactt

SEQ ID NO: 408 - DNA, 191 bp, Artificial
  Synthesized sequence
atatagatgccgtcctagcgaatccttgcgtcaatggttcgcagtggcacaagctgctgctggagggcggcggccaccaccgcgttgacttcaagaccatctacagggccaagaaggcggtgaagctgcccgactatcactttgtcactgccgtgtaaaatccgagaaccctgggcacaggaaagatactt

SEQ ID NO: 409 - DNA, 187 bp, Artificial
  Synthesized sequence
atatagatgccgtcctagcgaatccttgcgtcaatggttcgcagtgaagctgcccgactatcactttgtggaccaccgcatcgagatcctgaaccacgacaaggactacaacaaggtgaccgtttacgagagcgccgtggccactgccgtgtaaaatccgagaaccctgggcacaggaaagatactt

SEQ ID NO: 410 - DNA, 180 bp, Artificial
  Synthesized sequence
atatagatgccgtcctagcgaatccttgcgtcaatggttcgcagtggtttacgagagcgccgtggcccgcaactccaccgacggcatggacgagctgtacaagtaaaagcttccgggattcagtgattgaacttcactgccgtgtaaaatccgagaaccctgggcacaggaaagatactt

SEQ ID NO: 411 - DNA, 185 bp, Artificial
  Synthesized sequence
atatagatgccgtcctagcgtgtcgtgcctctttatctgtgcagtgttgtcgagtcctatgtaaccgtggtaccatggtgagcaagggcgaggagctgttcaccggggtggtgcccatcctggtcgagctggacggcgacactgccatttccgatacaccgaagctgggcacaggaaagatactt

SEQ ID NO: 412 - DNA, 192 bp, Artificial
  Synthesized sequence
atatagatgccgtcctagcgtgtcgtgcctctttatctgtgcagtgggtcgagctggacggcgacgtaaacggccacaagttcagcgtgtccggcgagggcgagggcgatgccacctacggcaagctgaccctgaagttcatctgccactgccatttccgatacaccgaagctgggcacaggaaagatactt

SEQ ID NO: 413 - DNA, 188 bp, Artificial
  Synthesized sequence
atatagatgccgtcctagcgtgtcgtgcctctttatctgtgcagtgaagctgaccctgaagttcatctgcaccaccggcaagctgcccgtgccctggcccaccctcgtgaccaccttcggctacggcctgatgtgcttcgcccactgccatttccgatacaccgaagctgggcacaggaaagatactt

SEQ ID NO: 414 - DNA, 187 bp, Artificial
  Synthesized sequence
atatagatgccgtcctagcgtgtcgtgcctctttatctgtgcagtgacggcctgatgtgcttcgcccgctaccccgaccacatgaagcagcacgacttcttcaagtccgccatgcccgaaggctacgtccaggagcgcacccactgccatttccgatacaccgaagctgggcacaggaaagatactt

SEQ ID NO: 415 - DNA, 192 bp, Artificial
  Synthesized sequence
atatagatgccgtcctagcgtgtcgtgcctctttatctgtgcagtgctacgtccaggagcgcaccatcttcttcaaggacgacggcaactacaagacccgcgccgaggtgaagttcgagggcgacaccctggtgaaccgcatcgagcactgccatttccgatacaccgaagctgggcacaggaaagatactt

SEQ ID NO: 416 - DNA, 192 bp, Artificial
  Synthesized sequence
atatagatgccgtcctagcgtgtcgtgcctctttatctgtgcagtgaccctggtgaaccgcatcgagctgaagggcatcgacttcaaggaggacggcaacatcctggggcacaagctggagtacaactacaacagccacaacgtctcactgccatttccgatacaccgaagctgggcacaggaaagatactt

SEQ ID NO: 417 - DNA, 189 bp, Artificial
  Synthesized sequence
atatagatgccgtcctagcgtgtcgtgcctctttatctgtgcagtgacaactacaacagccacaacgtctatatcatggccgacaagcagaagaacggcatcaaggtgaacttcaagatccgccacaacatcgaggacggcagcactgccatttccgatacaccgaagctgggcacaggaaagatactt

SEQ ID NO: 418 - DNA, 192 bp, Artificial
  Synthesized sequence
atatagatgccgtcctagcgtgtcgtgcctctttatctgtgcagtgccacaacatcgaggacggcagcgtgcagctcgccgaccactaccagcagaacacccccatcggcgacggccccgtgctgctgcccgacaaccactacctgcactgccatttccgatacaccgaagctgggcacaggaaagatactt

SEQ ID NO: 419 - DNA, 189 bp, Artificial
  Synthesized sequence
atatagatgccgtcctagcgtgtcgtgcctctttatctgtgcagtgctgcccgacaaccactacctgagctaccagtccaaactgagcaaagaccccaacgagaagcgcgatcacatggtcctgctggagttcgtgaccgccgcactgccatttccgatacaccgaagctgggcacaggaaagatactt

SEQ ID NO: 420 - DNA, 179 bp, Artificial
  Synthesized sequence
atatagatgccgtcctagcgtgtcgtgcctctttatctgtgcagtgtgctggagttcgtgaccgccgccgggatcactctcggcatggacgagctgtacaagtaaaagctttgaagatatgacgacccctgttcactgccatttccgatacaccgaagctgggcacaggaaagatactt

SEQ ID NO: 421 - DNA, 191 bp, Artificial
  Synthesized sequence
atatagatgccgtcctagcgatttaaacggtgaggtgtgcgcagtgttgtaagatggaagccgggataggtaccatggtgagcaagggcgaggagaataacatggccatcatcaaggagttcatgcgcttcaaggtgcacatggacactgctgatagccagcgaaacgatatgggcacaggaaagatactt

SEQ ID NO: 422 - DNA, 189 bp, Artificial
  Synthesized sequence
atatagatgccgtcctagcgatttaaacggtgaggtgtgcgcagtgtgcgcttcaaggtgcacatggagggctccgtgaacggccacgagttcgagatcgagggcgagggcgagggccgcccctacgaggcctttcagaccgccactgctgatagccagcgaaacgatatgggcacaggaaagatactt

SEQ ID NO: 423 - DNA, 191 bp, Artificial
  Synthesized sequence
atatagatgccgtcctagcgatttaaacggtgaggtgtgcgcagtgcctacgaggcctttcagaccgctaagctgaaggtgaccaagggtggccccctgcccttcgcctgggacatcctgtcccctcagttcatgtacggctccacactgctgatagccagcgaaacgatatgggcacaggaaagatactt

SEQ ID NO: 424 - DNA, 191 bp, Artificial
  Synthesized sequence
atatagatgccgtcctagcgatttaaacggtgaggtgtgcgcagtgcccctcagttcatgtacggctccaaggtctacattaagcacccagccgacatccccgactacttcaagctgtccttccccgagggcttcaggtgggagccactgctgatagccagcgaaacgatatgggcacaggaaagatactt

SEQ ID NO: 425 - DNA, 191 bp, Artificial
  Synthesized sequence
atatagatgccgtcctagcgatttaaacggtgaggtgtgcgcagtgccgagggcttcaggtgggagcgcgtgatgaacttcgaggacggcggcattattcacgttaaccaggactcctccctgcaggacggcgtgttcatctacacactgctgatagccagcgaaacgatatgggcacaggaaagatactt

SEQ ID NO: 426 - DNA, 187 bp, Artificial
  Synthesized sequence
atatagatgccgtcctagcgatttaaacggtgaggtgtgcgcagtgcaggacggcgtgttcatctacaaggtgaagctgcgcggcaccaacttcccctccgacggccccgtaatgcagaaaaagaccatgggctgggaggccactgctgatagccagcgaaacgatatgggcacaggaaagatactt

SEQ ID NO: 427 - DNA, 189 bp, Artificial
  Synthesized sequence
atatagatgccgtcctagcgatttaaacggtgaggtgtgcgcagtgaagaccatgggctgggaggcctccgaggagcggatgtaccccgaggacggcgccttaaagagcgagatcaaaaagaggctgaagctgaaggacggcgcactgctgatagccagcgaaacgatatgggcacaggaaagatactt

SEQ ID NO: 428 - DNA, 191 bp, Artificial
  Synthesized sequence
atatagatgccgtcctagcgatttaaacggtgaggtgtgcgcagtgaggctgaagctgaaggacggcggccactacgccgccgaggtcaagaccacctacaaggccaagaagcccgtgcagctgcccggcgcctacatcgtcgaccactgctgatagccagcgaaacgatatgggcacaggaaagatactt

SEQ ID NO: 429 - DNA, 192 bp, Artificial
  Synthesized sequence
atatagatgccgtcctagcgatttaaacggtgaggtgtgcgcagtgccggcgcctacatcgtcgacatcaagttggacatcgtgtcccacaacgaggactacaccatcgtggaacagtacgaacgcgccgagggccgccactccaccactgctgatagccagcgaaacgatatgggcacaggaaagatactt

SEQ ID NO: 430 - DNA, 166 bp, Artificial
  Synthesized sequence
atatagatgccgtcctagcgatttaaacggtgaggtgtgcgcagtgcgagggccgccactccaccggcggcatggacgagctgtacaagtaaaagcttttccacagctctatgaggtgttcactgctgatagccagcgaaacgatatgggcacaggaaagatactt

SEQ ID NO: 431 - DNA, 189 bp, Artificial
  Synthesized sequence
atatagatgccgtcctagcgcatccgatggtggtgtagatgctcttcttttggtgtcgcaacatgatctacggtaccatggtgagcaagggcgaggagaataacatggccatcatcaaggagttcatgcgcttcaaggtgcagaagagcggagaacggtcaactatccatgggcacaggaaagatactt

SEQ ID NO: 432 - DNA, 189 bp, Artificial
  Synthesized sequence
atatagatgccgtcctagcgcatccgatggtggtgtagatgctcttccaaggagttcatgcgcttcaaggtgcacatggagggctccgtgaacggccacgagttcgagatcgagggcgagggcgagggccgcccctacgagggaagagcggagaacggtcaactatccatgggcacaggaaagatactt

SEQ ID NO: 433 - DNA, 186 bp, Artificial
  Synthesized sequence
atatagatgccgtcctagcgcatccgatggtggtgtagatgctcttcggcgagggccgcccctacgaggcctttcagaccgctaagctgaaggtgaccaagggtggccccctgcccttcgcctgggacatcctgtccccgaagagcggagaacggtcaactatccatgggcacaggaaagatactt

SEQ ID NO: 434 - DNA, 190 bp, Artificial
  Synthesized sequence
atatagatgccgtcctagcgcatccgatggtggtgtagatgctcttccttcgcctgggacatcctgtcccctcagttcatgtacggctccaaggtctacattaagcacccagccgacatccccgactacttcaagctgtccttgaagagcggagaacggtcaactatccatgggcacaggaaagatactt

SEQ ID NO: 435 - DNA, 190 bp, Artificial
  Synthesized sequence
atatagatgccgtcctagcgcatccgatggtggtgtagatgctcttccatccccgactacttcaagctgtccttccccgagggcttcaggtgggagcgcgtgatgaacttcgaggacggcggcattattcacgttaaccaggagaagagcggagaacggtcaactatccatgggcacaggaaagatactt

SEQ ID NO: 436 - DNA, 190 bp, Artificial
  Synthesized sequence
atatagatgccgtcctagcgcatccgatggtggtgtagatgctcttcacggcggcattattcacgttaaccaggactcctccctgcaggacggcgtgttcatctacaaggtgaagctgcgcggcaccaacttcccctccgacggaagagcggagaacggtcaactatccatgggcacaggaaagatactt

SEQ ID NO: 437 - DNA, 187 bp, Artificial
  Synthesized sequence
atatagatgccgtcctagcgcatccgatggtggtgtagatgctcttccgcggcaccaacttcccctccgacggccccgtaatgcagaaaaagaccatgggctgggaggcctccgaggagcggatgtaccccgaggacggcgaagagcggagaacggtcaactatccatgggcacaggaaagatactt

SEQ ID NO: 438 - DNA, 184 bp, Artificial
  Synthesized sequence
atatagatgccgtcctagcgcatccgatggtggtgtagatgctcttcggagcggatgtaccccgaggacggcgccttaaagagcgagatcaaaaagaggctgaagctgaaggacggcggccactacgccgccgaggtgaagagcggagaacggtcaactatccatgggcacaggaaagatactt

SEQ ID NO: 439 - DNA, 190 bp, Artificial
  Synthesized sequence
atatagatgccgtcctagcgcatccgatggtggtgtagatgctcttcgcggccactacgccgccgaggtcaagaccacctacaaggccaagaagcccgtgcagctgcccggcgcctacatcgtcgacatcaagttggacatcggaagagcggagaacggtcaactatccatgggcacaggaaagatactt

SEQ ID NO: 440 - DNA, 190 bp, Artificial
  Synthesized sequence
atatagatgccgtcctagcgcatccgatggtggtgtagatgctcttctacatcgtcgacatcaagttggacatcgtgtcccacaacgaggactacaccatcgtggaacagtacgaacgcgccgagggccgccactccaccggcgaagagcggagaacggtcaactatccatgggcacaggaaagatactt

SEQ ID NO: 441 - DNA, 170 bp, Artificial
  Synthesized sequence
atatagatgccgtcctagcgcatccgatggtggtgtagatgctcttccgagggccgccactccaccggcggcatggacgagctgtacaagtaaaagcttgcaaacatgactaggaaccgttttgaagagcggagaacggtcaactatccatgggcacaggaaagatactt

SEQ ID NO: 442 - DNA, 187 bp, Artificial
  Synthesized sequence
ccctttaatcagatgcgtcgcttaaaccggccaacataccgcagtgttgcttattcgtgccgtgttatggcccagccggccaggcgcgaagtgcagctggtggagtcaggcggtggactggtgcagccaggaggttccctgcactgctcgaaaggaacgagtagcatggtcgcccttattactacca

SEQ ID NO: 443 - DNA, 187 bp, Artificial
  Synthesized sequence
ccctttaatcagatgcgtcgcttaaaccggccaacataccgcagtgtgcagccaggaggttccctgagactctcatgcgcagcaagcggttttaatatcaaggacacttatatacactgggtgcgccaagcccccggaaagcactgctcgaaaggaacgagtagcatggtcgcccttattactacca

SEQ ID NO: 444 - DNA, 187 bp, Artificial
  Synthesized sequence
ccctttaatcagatgcgtcgcttaaaccggccaacataccgcagtgcgccaagcccccggaaagggtctggagtgggtggccagaatataccccacaaacggctataccaggtacgcagattcagtgaaggggagattcaccactgctcgaaaggaacgagtagcatggtcgcccttattactacca

SEQ ID NO: 445 - DNA, 187 bp, Artificial
  Sythesized sequence
ccctttaatcagatgcgtcgcttaaaccggccaacataccgcagtgagattcagtgaaggggagattcaccataagcgctgacacatctaagaatactgcttacctgcaaatgaattccctgagggcagaggatacagctgcactgctcgaaaggaacgagtagcatggtcgcccttattactacca

SEQ ID NO: 446 - DNA, 188 bp, Artificial
  Synthesized sequence
ccctttaatcagatgcgtcgcttaaaccggccaacataccgcagtgctgagggcagaggatacagctgtttattactgcagccggtggggcggagatggcttttacgccatggactattgggggcagggaaccctggtcacccactgctcgaaaggaacgagtagcatggtcgcccttattactacca

SEQ ID NO: 447 - DNA, 187 bp, Artificial
  Synthesized sequence
ccctttaatcagatgcgtcgcttaaaccggccaacataccgcagtgggcagggaaccctggtcaccgtttccagcggtgggtcagggggcagcggcggcgccagcggagcagggagcggtggaggcgatatccaaatgacacactgctcgaaaggaacgagtagcatggtcgcccttattactacca

SEQ ID NO: 448 - DNA, 184 bp, Artificial
  Synthesized sequence
ccctttaatcagatgcgtcgcttaaaccggccaacataccgcagtgggtggaggcgatatccaaatgacacagtccccctctagcctgagcgccagcgtcggtgacagggtgaccattacatgcagggcctctcaggacactgctcgaaaggaacgagtagcatggtcgcccttattactacca

SEQ ID NO: 449 - DNA, 187 bp, Artificial
  Synthesized sequence
ccctttaatcagatgcgtcgcttaaaccggccaacataccgcagtgtacatgcagggcctctcaggatgttaatactgccgttgcatggtaccagcagaagcccgggaaggcaccaaagctgctgatctattccgcttcctcactgctcgaaaggaacgagtagcatggtcgcccttattactacca

SEQ ID NO: 450 - DNA, 189 bp, Artificial
  Synthesized sequence
ccctttaatcagatgcgtcgcttaaaccggccaacataccgcagtgagctgctgatctattccgcttcctttctgtacagcggagtgcctagcaggttttccggatctcgcagcggaactgattttacactcaccatcagcagcactgctcgaaaggaacgagtagcatggtcgcccttattactacca

SEQ ID NO: 451 - DNA, 185 bp, Artificial
  Synthesized sequence
ccctttaatcagatgcgtcgcttaaaccggccaacataccgcagtgactgattttacactcaccatcagcagcctccaacctgaggattttgccacctattattgccagcaacactacaccactccacccactttcggccactgctcgaaaggaacgagtagcatggtcgcccttattactacca

SEQ ID NO: 452 - DNA, 166 bp, Artificial
  Synthesized sequence
ccctttaatcagatgcgtcgcttaaaccggccaacataccgcagtgcaccactccacccactttcggccagggaactaaggtggaaataaaagggcccgggcacagcaatcaaaagtattcactgctcgaaaggaacgagtagcatggtcgcccttattactacca

SEQ ID NO: 453 - DNA, 187 bp, Artificial
  Synthesized sequence
ccctttaatcagatgcgtcgtgctctttattcgttgcgtcgcagtgtttttgcttcagtcagattcgcggcccagccggccaggcgccaggttcagctcaagcagtctggacccggactggtgcagccctctcagtctctccactgcagaacgaagcaccgataagaggtcgcccttattactacca

SEQ ID NO: 454 - DNA, 187 bp, Artificial
  Synthesized sequence
ccctttaatcagatgcgtcgtgctctttattcgttgcgtcgcagtggtgcagccctctcagtctctctctatcacctgcacagtgtctggtttctctctcaccaactacggggtccattgggttcggcagtccccagggaacactgcagaacgaagcaccgataagaggtcgcccttattactacca

SEQ ID NO: 455 - DNA, 187 bp, Artificial
  Synthesized sequence
ccctttaatcagatgcgtcgtgctctttattcgttgcgtcgcagtgtcggcagtccccagggaaagggctcgaatggctgggcgtgatctggtccggcggcaataccgactacaacaccccatttacttccaggctgtcaacactgcagaacgaagcaccgataagaggtcgcccttattactacca

SEQ ID NO: 456 - DNA, 187 bp, Artificial
  Synthesized sequence
ccctttaatcagatgcgtcgtgctctttattcgttgcgtcgcagtgccccatttacttccaggctgtcaattaataaggacaattctaagagccaggtcttctttaagatgaactctctccagtctaatgatactgccatccactgcagaacgaagcaccgataagaggtcgcccttattactacca

SEQ ID NO: 457 - DNA, 187 bp, Artificial
  Synthesized sequence
ccctttaatcagatgcgtcgtgctctttattcgttgcgtcgcagtgtctccagtctaatgatactgccatctactactgtgcccgggcactcacatactacgattatgaattcgcttactggggccagggcaccctcgtcacactgcagaacgaagcaccgataagaggtcgcccttattactacca

SEQ ID NO: 458 - DNA, 184 bp, Artificial
  Synthesized sequence
ccctttaatcagatgcgtcgtgctctttattcgttgcgtcgcagtgggccagggcaccctcgtcaccgtgagcgcaggaggatctgctggctctgggtcaagcggtggcgcttccggctcagggggagacatcctgctcactgcagaacgaagcaccgataagaggtcgcccttattactacca

SEQ ID NO: 459 - DNA, 187 bp, Artificial
  Synthesized sequence
ccctttaatcagatgcgtcgtgctctttattcgttgcgtcgcagtggctcagggggagacatcctgctcacccagagccccgtgattctgtccgttagccccggagaacgcgtttcttttagctgtcgcgcatctcagagccactgcagaacgaagcaccgataagaggtcgcccttattactacca

SEQ ID NO: 460 - DNA, 187 bp, Artificial
  Synthesized sequence
ccctttaatcagatgcgtcgtgctctttattcgttgcgtcgcagtgagctgtcgcgcatctcagagcatcggtaccaacattcactggtatcagcagcggaccgacgggagccctcgcctcctgataaaatatgcttctgacactgcagaacgaagcaccgataagaggtcgcccttattactacca

SEQ ID NO: 461 - DNA, 187 bp, Artificial
  Synthesized sequence
ccctttaatcagatgcgtcgtgctctttattcgttgcgtcgcagtgtcgcctcctgataaaatatgcttctgagtcaattagcggtatcccctccagatttagcgggagcggttctgggaccgatttcacactgagcatcacactgcagaacgaagcaccgataagaggtcgcccttattactacca

SEQ ID NO: 462 - DNA, 186 bp, Artificial
  Synthesized sequence
ccctttaatcagatgcgtcgtgctctttattcgttgcgtcgcagtgggaccgatttcacactgagcatcaactctgtggagtctgaagatatcgctgattattactgtcagcaaaacaacaattggcctaccaccttcggcactgcagaacgaagcaccgataagaggtcgcccttattactacca

SEQ ID NO: 463 - DNA, 168 bp, Artificial
  Synthesized sequence
ccctttaatcagatgcgtcgtgctctttattcgttgcgtcgcagtgaacaattggcctaccaccttcggcgccggcaccaagctggaactgaaagggcccccgggattcagtgattgaacttcactgcagaacgaagcaccgataagaggtcgcccttattactacca

SEQ ID NO: 464 - DNA, 185 bp, Artificial
  Synthesized sequence
ccctttaatcagatgcgtcgtgagccttatgatttcccgtgcagtgttgtcgagtcctatgtaaccgtggcccagccggccaggcgccaagttcagctccaggagtcaggtcctggtctggtgagaccatcccagaccccactgcgctcattcaggaaaacggacggtcgcccttattactacca

SEQ ID NO: 465 - DNA, 185 bp, Artificial
  Synhesized sequence
ccctttaatcagatgcgtcgtgagccttatgatttcccgtgcagtgctggtgagaccatcccagaccctctctctcacttgtaccgtttccggcttcacattcaccgatttctatatgaactgggttaggcaaccaccacactgcgctcattcaggaaaacggacggtcgcccttattactacca

SEQ ID NO: 466 - DNA, 187 bp, Artificial
  Synthesized sequence
ccctttaatcagatgcgtcgtgagccttatgatttcccgtgcagtggaactgggttaggcaaccaccaggccgggggctggaatggatcggttttatcagagataaagccaagggatatactactgagtacaacccctctgcactgcgctcattcaggaaaacggacggtcgcccttattactacca

SEQ ID NO: 467 - DNA, 187 bp, Artificial
  Synthesized sequence
ccctttaatcagatgcgtcgtgagccttatgatttcccgtgcagtgatactactgagtacaacccctctgtgaagggtcgggtgaccatgctggttgacacaagcaagaatcaattttcactccggctgtcatctgtgacacactgcgctcattcaggaaaacggacggtcgcccttattactacca

SEQ ID NO: 468 - DNA, 186 bp, Artificial
  Synthesized sequence
ccctttaatcagatgcgtcgtgagccttatgatttcccgtgcagtgctccggctgtcatctgtgacagctgctgatacagcagtttattattgcgcaagggaaggacatactgccgctcctttcgactattggggccaggcactgcgctcattcaggaaaacggacggtcgcccttattactacca

SEQ ID NO: 469 - DNA, 183 bp, Artificial
  Synthesized sequence
ccctttaatcagatgcgtcgtgagccttatgatttcccgtgcagtgtcctttcgactattggggccagggttcactcgtcacagtctcttcaggtggggccggctcaggagccgggagcgggtcatctggagccggccactgcgctcattcaggaaaacggacggtcgcccttattactacca

SEQ ID NO: 470 - DNA, 187 bp, Artificial
  Synthesized sequence
ccctttaatcagatgcgtcgtgagccttatgatttcccgtgcagtggcgggtcatctggagccggctccggggatatccagatgacccagtcaccctcttcactcagcgccagcgtgggcgatcgcgttaccatcacatgccactgcgctcattcaggaaaacggacggtcgcccttattactacca

SEQ ID NO: 471 - DNA, 187 bp, Artificial
  Synthesized sequence
ccctttaatcagatgcgtcgtgagccttatgatttcccgtgcagtgggcgatcgcgttaccatcacatgcaaagcttctcagaacattgacaaatacctgaattggtaccaacagaagcccggcaaggcccccaaactcctcactgcgctcattcaggaaaacggacggtcgcccttattactacca

SEQ ID NO: 472 - DNA, 187 bp, Artificial
  Synthesized sequence
ccctttaatcagatgcgtcgtgagccttatgatttcccgtgcagtgggcaaggcccccaaactcctcatatacaatacaaacaatctgcagaccggcgtgccatcccgcttctcaggatcaggcagcggcactgactttaccactgcgctcattcaggaaaacggacggtcgcccttattactacca

SEQ ID NO: 473 - DNA, 187 bp, Artificial
  Synthesized sequence
ccctttaatcagatgcgtcgtgagccttatgatttcccgtgcagtgggcagcggcactgactttactttcacaatcagcagcctgcaaccagaggacatcgccacatattactgtctccagcatatctcccgccctcggaccactgcgctcattcaggaaaacggacggtcgcccttattactacca

SEQ ID NO: 474 - DNA, 172 bp, Artificial
  Synthesized sequence
ccctttaatcagatgcgtcgtgagccttatgatttcccgtgcagtggcatatctcccgccctcggacattcggccaaggtacaaaggtggagattaaagggccctgaagatatgacgacccctgttcactgcgctcattcaggaaaacggacggtcgcccttattactacca

SEQ ID NO: 475 - DNA, 187 bp, Artificial
  Synthesized sequence
ccctttaatcagatgcgtcgcgttctaaacggctagatgcgcagtgttgtaagatggaagccgggataggcccagccggccaggcgcgaagtgcaactggttgaaagcggtgggggcctggtgcagcctggtggatcactgcactgcggaaaggggaaagacagactggtcgcccttattactacca

SEQ ID NO: 476 - DNA, 187 bp, Artificial
  Synthesized sequence
ccctttaatcagatgcgtcgcgttctaaacggctagatgcgcagtggtgcagcctggtggatcactgagactctcctgcgccgccagcggttacaccttcaccaactatggtatgaattgggttagacaagcacctggaaacactgcggaaaggggaaagacagactggtcgcccttattactacca

SEQ ID NO: 477 - DNA, 186 bp, Artificial
  Synthesized sequence
ccctttaatcagatgcgtcgcgttctaaacggctagatgcgcagtgtgggttagacaagcacctggaaagggactggagtgggttggctggataaatacatatacaggcgagccaacatatgcagctgactttaagcggacactgcggaaaggggaaagacagactggtcgcccttattactacca

SEQ ID NO: 478 - DNA, 184 bp, Artificial
  Synthesized sequence
ccctttaatcagatgcgtcgcgttctaaacggctagatgcgcagtgatatgcagctgactttaagcggaggtttaccttctcactggacacatccaagtctactgcttacctgcagatgaactcactccgggctgaggcactgcggaaaggggaaagacagactggtcgcccttattactacca

SEQ ID NO: 479 - DNA, 187 bp, Artificial
  Synthesized sequence
ccctttaatcagatgcgtcgcgttctaaacggctagatgcgcagtgtgaactcactccgggctgaggatacagccgtttactattgcgccaagtatccccattactatggttccagccactggtacttcgatgtctggggccactgcggaaaggggaaagacagactggtcgcccttattactacca

SEQ ID NO: 480 - DNA, 182 bp, Artificial
  Synthesized sequence
ccctttaatcagatgcgtcgcgttctaaacggctagatgcgcagtgcactggtacttcgatgtctggggccagggaactctggtgactggggggtccgggggctccggaggggcctccggagcaggatccggcggacactgcggaaaggggaaagacagactggtcgcccttattactacca

SEQ ID NO: 481 - DNA, 187 bp, Artificial
  Synthesized sequence
ccctttaatcagatgcgtcgcgttctaaacggctagatgcgcagtgcggagcaggatccggcggaggtgacatacagatgacccagtctccatcctctctgagcgcctctgtgggcgatcgcgtcactattacctgttctgcactgcggaaaggggaaagacagactggtcgcccttattactacca

SEQ ID NO: 482 - DNA, 187 bp, Artificial
  Synthesized sequence
ccctttaatcagatgcgtcgcgttctaaacggctagatgcgcagtgatcgcgtcactattacctgttctgcatctcaggatattagcaactatctgaattggtatcagcagaagccaggtaaggcaccaaaagttctgatccactgcggaaaggggaaagacagactggtcgcccttattactacca

SEQ ID NO: 483 - DNA, 187 bp, Artificial
  Synthesized sequence
ccctttaatcagatgcgtcgcgttctaaacggctagatgcgcagtgaggtaaggcaccaaaagttctgatctacttcacaagctctctgcattccggggtgccctcacgcttctctggttccggctccgggacagatttcacactgcggaaaggggaaagacagactggtcgcccttattactacca

SEQ ID NO: 484 - DNA, 187 bp, Artificial
  Synthesized sequence
ccctttaatcagatgcgtcgcgttctaaacggctagatgcgcagtgccggctccgggacagatttcacactcacaatttcctctctgcagcccgaagattttgcaacttactactgtcagcagtattctacagtgccatggcactgcggaaaggggaaagacagactggtcgcccttattactacca

SEQ ID NO: 485 - DNA, 177 bp, Artificial
  Synthesized sequence
ccctttaatcagatgcgtcgcgttctaaacggctagatgcgcagtgcagcagtattctacagtgccatggactttcggacagggaaccaaggtcgagattaaagggcccttccacagctctatgaggtgttcactgcggaaaggggaaagacagactggtcgcccttattactacca

SEQ ID NO: 486 - DNA, 188 bp, Artificial
  Synthesized sequence
ccctttaatcagatgcgtcggtatccgaagcgtggagtatgcagtgttggtgtcgcaacatgatctacggcccagccggccaggcgcgaagttcagctggttgaaagcggaggtggactcgtgcagcccggtgggtccctgacactgcttgactcctacgcatacctgggtcgcccttattactacca

SEQ ID NO: 487 - DNA, 188 bp, Artificial
  Synthesized sequence
ccctttaatcagatgcgtcggtatccgaagcgtggagtatgcagtgagcccggtgggtccctgaggctctcctgcgccgctagcggatatgatttcactcactacggtatgaattgggtccggcaggctcccggcaaaggtccactgcttgactcctacgcatacctgggtcgcccttattactacca

SEQ ID NO: 488 - DNA, 188 bp, Artificial
  Synthesized sequence
ccctttaatcagatgcgtcggtatccgaagcgtggagtatgcagtgcaggctcccggcaaaggtctggaatgggttggctggatcaacacttatactggggagcctacctacgccgccgatttcaagaggcgctttactttccactgcttgactcctacgcatacctgggtcgcccttattactacca

SEQ ID NO: 489 - DNA, 188 bp, Artificial
  Synthesized sequence
ccctttaatcagatgcgtcggtatccgaagcgtggagtatgcagtggatttcaagaggcgctttactttctcactcgatacctccaaatccacagcctatctgcaaatgaattccctgcgcgccgaagataccgcagtctaccactgcttgactcctacgcatacctgggtcgcccttattactacca

SEQ ID NO: 490 - DNA, 188 bp, Artificial
  Synthesized sequence
ccctttaatcagatgcgtcggtatccgaagcgtggagtatgcagtgcgccgaagataccgcagtctactattgtgccaagtatccctactattatgggacatctcactggtacttcgacgtgtgggggcaagggactctcgtcactgcttgactcctacgcatacctgggtcgcccttattactacca

SEQ ID NO: 491 - DNA, 188 bp, Artificial
  Synthesized sequence
ccctttaatcagatgcgtcggtatccgaagcgtggagtatgcagtgtgggggcaagggactctcgtcactgtgtctagcgggggtagcgctgggtccggcagcagcggtggggcaagcggtagcgggggcgacattcagctgcactgcttgactcctacgcatacctgggtcgcccttattactacca

SEQ ID NO: 492 - DNA, 188 bp, Artificial
  Synthesized sequence
ccctttaatcagatgcgtcggtatccgaagcgtggagtatgcagtggcgggggcgacattcagctgacacaaagcccctcatccctgagcgcttcagtgggggaccgcgtgaccatcacctgttccgcctcccaggacatctcactgcttgactcctacgcatacctgggtcgcccttattactacca

SEQ ID NO: 493 - DNA, 189 bp, Artificial
  Synthesized sequence
ccctttaatcagatgcgtcggtatccgaagcgtggagtatgcagtgttccgcctcccaggacatctcaaactacctgaactggtaccaacaaaaacctggtaaagcccctaaagttctgatttacttcacaagctctctccaccactgcttgactcctacgcatacctgggtcgcccttattactacca

SEQ ID NO: 494 - DNA, 188 bp, Artificial
  Synthesized sequence
ccctttaatcagatgcgtcggtatccgaagcgtggagtatgcagtggatttacttcacaagctctctccactccggcgtcccttctaggttttctggtagcggtagcggaacagatttcactctgacaattagctccctccacactgcttgactcctacgcatacctgggtcgcccttattactacca

SEQ ID NO: 495 - DNA, 188 bp, Artificial
  Synthesized sequence
ccctttaatcagatgcgtcggtatccgaagcgtggagtatgcagtgcactctgacaattagctccctccagcctgaggattttgccacttactattgtcagcagtattccacagtgccctggacttttgggcagggcaccaacactgcttgactcctacgcatacctgggtcgcccttattactacca

SEQ ID NO: 496 - DNA, 153 bp, Artificial
  Synthesized sequence
ccctttaatcagatgcgtcggtatccgaagcgtggagtatgcagtgacttttgggcagggcaccaaggtcgaaatcaaggggcccgcaaacatgactaggaaccgttcactgcttgactcctacgcatacctgggtcgcccttattactacca

SEQ ID NO: 497 - DNA, 182 bp, Artificial
  Synthesized sequence
ccctttaatcagatgcgtcgcttgttatggacgagttgccgcagtgttgtgctaagtcacactgttggggcccagccggccaggcgcgaggtccagctggtcgagagcggcggcgggctggttcaacccgggggctcactgccagtatgaacgcgccattaaggtcgcccttattactacca

SEQ ID NO: 498 - DNA, 182 bp, Artificial
  Synthesized sequence
ccctttaatcagatgcgtcgcttgttatggacgagttgccgcagtgctggttcaacccgggggctccctgcggctgtcatgtgccgccagcggcttcacctttactgattacacaatggactgggtgaggcaggcccactgccagtatgaacgcgccattaaggtcgcccttattactacca

SEQ ID NO: 499 - DNA, 190 bp, Artificial
  Synthesized sequence
ccctttaatcagatgcgtcgcttgttatggacgagttgccgcagtgtggactgggtgaggcaggccccaggaaaaggcctggaatgggttgccgacgtgaatcctaattccgggggttcaatttacaatcagcgctttaagggccactgccagtatgaacgcgccattaaggtcgcccttattactacca

SEQ ID NO: 500 - DNA, 184 bp, Artificial
  Synthesized sequence
ccctttaatcagatgcgtcgcttgttatggacgagttgccgcagtgtcaatttacaatcagcgctttaagggccggttcaccctgtcagtcgacaggagcaagaatacactctatctccagatgaactccctccgcgccactgccagtatgaacgcgccattaaggtcgcccttattactacca

SEQ ID NO: 501 - DNA, 187 bp, Artificial
  Synthesized sequence
ccctttaatcagatgcgtcgcttgttatggacgagttgccgcagtgccagatgaactccctccgcgctgaggataccgccgtctattattgtgcccgcaatctgggtccctctttttactttgactattggggccaagggacactgccagtatgaacgcgccattaaggtcgcccttattactacca

SEQ ID NO: 502 - DNA, 187 bp, Artificial
  Synthesized sequence
ccctttaatcagatgcgtcgcttgttatggacgagttgccgcagtgactttgactattggggccaagggaccctggtcaccgtctctagcgccggtggctcaggaggaagcggtggcgcctctggggctggcagcggaggacactgccagtatgaacgcgccattaaggtcgcccttattactacca

SEQ ID NO: 503 - DNA, 188 bp, Artificial
  Synthesized sequence
ccctttaatcagatgcgtcgcttgttatggacgagttgccgcagtgggggctggcagcggaggaggcgacattcagatgacacagagccctagctctctctccgctagcgtgggggacagggttaccataacttgcaaggcacactgccagtatgaacgcgccattaaggtcgcccttattactacca

SEQ ID NO: 504 - DNA, 187 bp, Artificial
  Synthesized sequence
ccctttaatcagatgcgtcgcttgttatggacgagttgccgcagtgcagggttaccataacttgcaaggcaagccaagatgtctctattggtgttgcttggtaccagcaaaagcctggaaaggctcctaaactgctgatatcactgccagtatgaacgcgccattaaggtcgcccttattactacca

SEQ ID NO: 505 - DNA, 187 bp, Artificial
  Synthesized sequence
ccctttaatcagatgcgtcgcttgttatggacgagttgccgcagtggaaaggctcctaaactgctgatatactccgccagctacaggtatacaggcgtgccatcccggttctcaggttccggctcaggaacagattttactcactgccagtatgaacgcgccattaaggtcgcccttattactacca

SEQ ID NO: 506 - DNA, 187 bp, Artificial
  Synthesized sequence
ccctttaatcagatgcgtcgcttgttatggacgagttgccgcagtgtccggctcaggaacagattttactctcaccatttccagcctgcaacccgaggacttcgccacatactattgccagcagtattatatatatccttacactgccagtatgaacgcgccattaaggtcgcccttattactacca

SEQ ID NO: 507 - DNA, 183 bp, Artificial
  Synthesized sequence
ccctttaatcagatgcgtcgcttgttatggacgagttgccgcagtgtattgccagcagtattatatatatccttacacttttggtcagggtactaaagtggagattaaagggcccccgggacgagattagtacaattcactgccagtatgaacgcgccattaaggtcgcccttattactacca

SEQ ID NO: 508 - DNA, 187 bp, Artificial
  Synthesized sequence
ccctttaatcagatgcgtcgccaaagattcaaccgtcctggcagtgtttctaaacagttaggcccaggggcccagccggccaggcgcgaggtgcagctccaacaatctgggcctgatctggttaagccaggcgcttctgtgcactgctccgtcctgaaatggctaatggtcgcccttattactacca

SEQ ID NO: 509 - DNA, 186 bp, Artificial
  Synthesized sequence
ccctttaatcagatgcgtcgccaaagattcaaccgtcctggcagtgggttaagccaggcgcttctgtgaaaatttcctgtaaggcttcaggctacagcttcactggctattatatgcattgggtgaaacagtctccaggacactgctccgtcctgaaatggctaatggtcgcccttattactacca

SEQ ID NO: 510 - DNA, 187 bp, Artificial
  Synthesized sequence
ccctttaatcagatgcgtcgccaaagattcaaccgtcctggcagtgattgggtgaaacagtctccaggaaagggcctggagtggattgggcggatcaatcccaacaatggagtcaccctctacaatcaaaaattcaaagatcactgctccgtcctgaaatggctaatggtcgcccttattactacca

SEQ ID NO: 511 - DNA, 187 bp, Artificial
  Synthesized sequence
ccctttaatcagatgcgtcgccaaagattcaaccgtcctggcagtgtcaccctctacaatcaaaaattcaaagataaagctacactgaccgtcgataaaagctcaacaacagcctacatggagctgagatccctcacctcccactgctccgtcctgaaatggctaatggtcgcccttattactacca

SEQ ID NO: 512 - DNA, 187 bp, Artificial
  Synthesized sequence
ccctttaatcagatgcgtcgccaaagattcaaccgtcctggcagtgtggagctgagatccctcacctccgaggacagcgctgtctactactgcgccaggtccacaatgattaccaattatgtgatggactactggggtcagcactgctccgtcctgaaatggctaatggtcgcccttattactacca

SEQ ID NO: 513 - DNA, 188 bp, Artificial
  Synthesized sequence
ccctttaatcagatgcgtcgccaaagattcaaccgtcctggcagtgatgtgatggactactggggtcagggaacctcagtgaccgttagctctggcgggtccgcaggtagcggctcatccggcggcgcatccgggagcggagcactgctccgtcctgaaatggctaatggtcgcccttattactacca

SEQ ID NO: 514 - DNA, 187 bp, Artificial
  Synthesized sequence
ccctttaatcagatgcgtcgccaaagattcaaccgtcctggcagtggcgcatccgggagcggagggtctattgtcatgacacagacccccacttccctcctggtctctgctggcgacagagtcacaatcacttgcaaggctcactgctccgtcctgaaatggctaatggtcgcccttattactacca

SEQ ID NO: 515 - DNA, 186 bp, Artificial
  Synthesized sequence
ccctttaatcagatgcgtcgccaaagattcaaccgtcctggcagtgagagtcacaatcacttgcaaggctagccagagcgtttcaaacgacgtggcatggtatcaacagaaacccggccaatcccccaaactgctgatttcactgctccgtcctgaaatggctaatggtcgcccttattactacca

SEQ ID NO: 516 - DNA, 187 bp, Artificial
  Synthesized sequence
ccctttaatcagatgcgtcgccaaagattcaaccgtcctggcagtgccaatcccccaaactgctgatttcttacacatcatccagatacgccggtgtgcccgataggttttctggttcagggtatggaactgacttcactccactgctccgtcctgaaatggctaatggtcgcccttattactacca

SEQ ID NO: 517 - DNA, 187 bp, Artificial
  Synthesized sequence
ccctttaatcagatgcgtcgccaaagattcaaccgtcctggcagtgcagggtatggaactgacttcactctcactatctctagcgttcaggctgaagacgctgccgtctacttctgccagcaagactacaactctcctcctcactgctccgtcctgaaatggctaatggtcgcccttattactacca

SEQ ID NO: 518 - DNA, 177 bp, Artificial
  Synthesized sequence
ccctttaatcagatgcgtcgccaaagattcaaccgtcctggcagtgcagcaagactacaactctcctcctacattcggcgggggcacaaagctggagatcaaagggccccacgccagttgtgaacataattcactgctccgtcctgaaatggctaatggtcgcccttattactacca

SEQ ID NO: 519 - DNA, 186 bp, Artificial
  Synthesized sequence
ccctttaatcagatgcgtcgtattcatgcttggacggactgcagtgttgtctttatacttgcctgccgggcccagccggccaggcgccaggtgcagctggtgcagtccggagccgaggtcaagaagcccggatcttccgtcactgctccaacaagcggtacatagtggtcgcccttattactacca

SEQ ID NO: 520 - DNA, 182 bp, Artificial
  Synthesized sequence
ccctttaatcagatgcgtcgtattcatgcttggacggactgcagtggtcaagaagcccggatcttccgtcaaagtcagctgcaaagcttccggttatgcattcactaactacctcatcgagtgggtccgccaggctcactgctccaacaagcggtacatagtggtcgcccttattactacca

SEQ ID NO: 521 - DNA, 191 bp, Artificial
  Synthesized sequence
ccctttaatcagatgcgtcgtattcatgcttggacggactgcagtgcgagtgggtccgccaggctccaggacagggactggagtggattggagtgatctaccctggatcaggaggcacaaattataacgagaagtttaagggcagcactgctccaacaagcggtacatagtggtcgcccttattactacca

SEQ ID NO: 522 - DNA, 185 bp, Artificial
  Synthesized sequence
ccctttaatcagatgcgtcgtattcatgcttggacggactgcagtgcaaattataacgagaagtttaagggcagagtcactctgaccgtcgatgaatccacaaatacagcttacatggagctgtcatcactccggagcgcactgctccaacaagcggtacatagtggtcgcccttattactacca

SEQ ID NO: 523 - DNA, 187 bp, Artificial
  Synthesized sequence
ccctttaatcagatgcgtcgtattcatgcttggacggactgcagtggagctgtcatcactccggagcgaggacacagcagtttatttttgcgcacgccgcgatggcaattacgggtggttcgcctattgggggcagggtaccactgctccaacaagcggtacatagtggtcgcccttattactacca

SEQ ID NO: 524 - DNA, 187 bp, Artificial
  Synthesized sequence
ccctttaatcagatgcgtcgtattcatgcttggacggactgcagtgcgcctattgggggcagggtactctcgtcaccgtgtcatcaggtggggctggctccggggcaggttctggctcctccggagctggttcaggagacacactgctccaacaagcggtacatagtggtcgcccttattactacca

SEQ ID NO: 525 - DNA, 183 bp, Artificial
  Synthesized sequence
ccctttaatcagatgcgtcgtattcatgcttggacggactgcagtgccggagctggttcaggagacatccagatgacccagacaccctccactctctctgcttctgtgggagacagagtcacaatcagctgccgggccactgctccaacaagcggtacatagtggtcgcccttattactacca

SEQ ID NO: 526 - DNA, 187 bp, Artificial
  Synthesized sequence
ccctttaatcagatgcgtcgtattcatgcttggacggactgcagtggtcacaatcagctgccgggcttcccaggatataaacaactacctgaactggtaccagcagaagcctgggaaggcccccaagctgctgatctactacactgctccaacaagcggtacatagtggtcgcccttattactacca

SEQ ID NO: 527 - DNA, 187 bp, Artificial
  Synthesized sequence
ccctttaatcagatgcgtcgtattcatgcttggacggactgcagtggcccccaagctgctgatctactatacatccactctgcacagcggagttcctagccgcttcagcggatccggtagcgggaccgactataccctgaccactgctccaacaagcggtacatagtggtcgcccttattactacca

SEQ ID NO: 528 - DNA, 185 bp, Artificial
  Synthesized sequence
ccctttaatcagatgcgtcgtattcatgcttggacggactgcagtggcgggaccgactataccctgaccatctcaagcctgcagcccgatgacttcgccacatacttctgtcagcagggaaacaccctcccatggacatcactgctccaacaagcggtacatagtggtcgcccttattactacca

SEQ ID NO: 529 - DNA, 171 bp, Artificial
  Synthesized sequence
ccctttaatcagatgcgtcgtattcatgcttggacggactgcagtgggaaacaccctcccatggacattcggtcaaggaactaaagttgaggttaaagggccccaaaggccaaatcagttccattcactgctccaacaagcggtacatagtggtcgcccttattactacca

SEQ ID NO: 530 - DNA, 186 bp, Artificial
  Synthesized sequence
ccctttaatcagatgcgtcgatcgacaatggtatggctgagcagtgttcaccgcgatcaatacaacttggcccagccggccaggcgcgaagttcaactggttgagagcggtgccgaggtgaagaagcctggagagtctctcactgcaggagtggctaggagacataggtcgcccttattactacca

SEQ ID NO: 531 - DNA, 187 bp, Artificial
  Synthesized sequence
ccctttaatcagatgcgtcgatcgacaatggtatggctgagcagtggtgaagaagcctggagagtctctgagaattagctgtaagggctctggctgcatcatctcatcttattggatttcatgggttagacagatgcccggcactgcaggagtggctaggagacataggtcgcccttattactacca

SEQ ID NO: 532 - DNA, 190 bp, Artificial
  Synthesized sequence
ccctttaatcagatgcgtcgatcgacaatggtatggctgagcagtgttcatgggttagacagatgcccggcaaaggactggaatggatgggcaagatagaccctggtgactcctacatcaattattccccttcttttcaggggccactgcaggagtggctaggagacataggtcgcccttattactacca

SEQ ID NO: 533 - DNA, 187 bp, Artificial
  Synthesized sequence
ccctttaatcagatgcgtcgatcgacaatggtatggctgagcagtgtcaattattccccttcttttcaggggcatgtcacaatctccgcagacaagagcatcaacacagcatatctccagtggaattcactgaaagcctcccactgcaggagtggctaggagacataggtcgcccttattactacca

SEQ ID NO: 534 - DNA, 186 bp, Artificial
  Synthesized sequence
ccctttaatcagatgcgtcgatcgacaatggtatggctgagcagtgcagtggaattcactgaaagcctccgacacagccatgtactattgcgcaagaggagggagggacttcggagactcttttgactactgggggcaggcactgcaggagtggctaggagacataggtcgcccttattactacca

SEQ ID NO: 535 - DNA, 182 bp, Artificial
  Synthesized sequence
ccctttaatcagatgcgtcgatcgacaatggtatggctgagcagtgctcttttgactactgggggcaggggactctggtgacagtgtctagcggcgggtcaggaggatccggtggagcctctggcgctggaagcggcactgcaggagtggctaggagacataggtcgcccttattactacca

SEQ ID NO: 536 - DNA, 187 bp, Artificial
  Synthesized sequence
ccctttaatcagatgcgtcgatcgacaatggtatggctgagcagtgcctctggcgctggaagcggcggcggagatgtggtcatgactcaatccccttcctttctgtcagcattcgtgggcgataggatcactattacttgtcactgcaggagtggctaggagacataggtcgcccttattactacca

SEQ ID NO: 537 - DNA, 187 bp, Artificial
  Synthesized sequence
ccctttaatcagatgcgtcgatcgacaatggtatggctgagcagtgtgggcgataggatcactattacttgtcgcgcctcttctggcatctccagatatctggcttggtaccagcaagctcccggaaaggcccctaagctgcactgcaggagtggctaggagacataggtcgcccttattactacca

SEQ ID NO: 538 - DNA, 184 bp, Artificial
  Synthesized sequence
ccctttaatcagatgcgtcgatcgacaatggtatggctgagcagtgcccggaaaggcccctaagctgctcatatatgccgcctccaccctccagactggagtgcccagccggtttagcggtagcggttccggtaccgacactgcaggagtggctaggagacataggtcgcccttattactacca

SEQ ID NO: 539 - DNA, 187 bp, Artificial
  Synthesized sequence
ccctttaatcagatgcgtcgatcgacaatggtatggctgagcagtgcggtagcggttccggtaccgagtttaccctcaccattaactctctgcagccagaagacttcgccacatattactgtcaacacctcaactcctatccactgcaggagtggctaggagacataggtcgcccttattactacca

SEQ ID NO: 540 - DNA, 182 bp, Artificial
  Synthesized sequence
ccctttaatcagatgcgtcgatcgacaatggtatggctgagcagtgactgtcaacacctcaactcctatcctctcactttcggcggcgggaccaaagtcgatattaaggggcccggtgcatgggaggaactatattcactgcaggagtggctaggagacataggtcgcccttattactacca

SEQ ID NO: 541 - DNA, 187 bp, Artificial
  Synthesized sequence
ccctttaatcagatgcgtcggtcctagtgaggaataccgggcagtgttttcggatagactcaggaagcggcccagccggccaggcgccaagttaaactgcaggagagcggagccgaactcgccagacccggagcttctgtgcactgctaggatctgcgattcttcggggtcgcccttattactacca

SEQ ID NO: 542 - DNA, 187 bp, Artificial
  Synthesized sequence
ccctttaatcagatgcgtcggtcctagtgaggaataccgggcagtgccagacccggagcttctgtgaaactgagctgcaaagcttctggctatacttttaccaattattggatgcaatgggtgaagcagaggccaggacagcactgctaggatctgcgattcttcggggtcgcccttattactacca

SEQ ID NO: 543 - DNA, 189 bp, Artificial
  Synthesized sequence
ccctttaatcagatgcgtcggtcctagtgaggaataccgggcagtggtgaagcagaggccaggacagggactggactggatcggagctatctatcctggagacggcaatactcggtacacacacaaatttaaggggaaagctacactgctaggatctgcgattcttcggggtcgcccttattactacca

SEQ ID NO: 544 - DNA, 187 bp, Artificial
  Synthesized sequence
ccctttaatcagatgcgtcggtcctagtgaggaataccgggcagtgcacacacaaatttaaggggaaagctaccctgaccgctgataagtcatcatctaccgcctacatgcagctgagctccctggcttcagaggacagcgcactgctaggatctgcgattcttcggggtcgcccttattactacca

SEQ ID NO: 545 - DNA, 182 bp, Artificial
  Synthesized sequence
ccctttaatcagatgcgtcgtgcaaatgtgaggtagcaacgcagtgtttcgaacaatttgcgatacccggcccagccggccaggcgcgaagtccaactggttcagtccgggggcggcctggtgaaacccggcggctcactgcaacgcaagcgaaaactacaaggtcgcccttattactacca

SEQ ID NO: 546 - DNA, 187 bp, Artificial
  Synthesized sequence
ccctttaatcagatgcgtcgtgcaaatgtgaggtagcaacgcagtgctggtgaaacccggcggctccctgaggctctcatgcgccgccagcggatttactttttcctcatttgccatgcactgggtgaggcaggcaccaggcactgcaacgcaagcgaaaactacaaggtcgcccttattactacca

SEQ ID NO: 547 - DNA, 187 bp, Artificial
  Synthesized sequence
ccctttaatcagatgcgtcgtgcaaatgtgaggtagcaacgcagtggggtgaggcaggcaccaggaaaaggactggagtggatcagcgtcattgatacaagaggtgcaacatattacgctgacagcgtgaaggggagatttcactgcaacgcaagcgaaaactacaaggtcgcccttattactacca

SEQ ID NO: 548 - DNA, 184 bp, Artificial
  Synthesized sequence
ccctttaatcagatgcgtcgtgcaaatgtgaggtagcaacgcagtgtgacagcgtgaaggggagatttacaattagccgcgataacgccaagaactccctgtacctgcagatgaactccctgcgggctgaagacacagcactgcaacgcaagcgaaaactacaaggtcgcccttattactacca

SEQ ID NO: 549 - DNA, 187 bp, Artificial
  Synthesized sequence
ccctttaatcagatgcgtcgtgcaaatgtgaggtagcaacgcagtgccctgcgggctgaagacacagccgtgtactattgtgcaaggctgggtaatttttattacggcatggacgtttgggggcaggggactactgtgacacactgcaacgcaagcgaaaactacaaggtcgcccttattactacca

SEQ ID NO: 550 - DNA, 185 bp, Artificial
  Synthesized sequence
ccctttaatcagatgcgtcgtgcaaatgtgaggtagcaacgcagtgggggcaggggactactgtgacagtttcctcaggggggagcggggggagcgggggggctagcggcgctggctccggagggggagagatcgtcctcactgcaacgcaagcgaaaactacaaggtcgcccttattactacca

SEQ ID NO: 551 - DNA, 183 bp, Artificial
  Synthesized sequence
ccctttaatcagatgcgtcgtgcaaatgtgaggtagcaacgcagtgccggagggggagagatcgtcctgacacagtcacccgggactctgtctgtgagccctggcgagagagcaactctgtcatgcagggccagccacactgcaacgcaagcgaaaactacaaggtcgcccttattactacca

SEQ ID NO: 552 - DNA, 187 bp, Artificial
  Synthesized sequence
ccctttaatcagatgcgtcgtgcaaatgtgaggtagcaacgcagtgctgtcatgcagggccagccaaagcatcggctcatctctgcactggtaccagcagaaacccggtcaggccccacgcctgctgatcaaatatgccagcactgcaacgcaagcgaaaactacaaggtcgcccttattactacca

SEQ ID NO: 553 - DNA, 187 bp, Artificial
  Synthesized sequence
ccctttaatcagatgcgtcgtgcaaatgtgaggtagcaacgcagtgacgcctgctgatcaaatatgccagccagagcctgtcaggcattcctgacagattttctgggagcggatcaggaacagatttcacactcacaatatcactgcaacgcaagcgaaaactacaaggtcgcccttattactacca

SEQ ID NO: 554 - DNA, 187 bp, Artificial
  Synthesized sequence
ccctttaatcagatgcgtcgtgcaaatgtgaggtagcaacgcagtgaggaacagatttcacactcacaatatccaggctggagcccgaagacttcgctgtctactactgccaccagtccagcagactccctcacaccttcgcactgcaacgcaagcgaaaactacaaggtcgcccttattactacca

SEQ ID NO: 555 - DNA, 188 bp, Artificial
  Synthesized sequence
ccctttaatcagatgcgtcggtcctagtgaggaataccgggcagtgtccctggcttcagaggacagcggcgtttactattgcgcacgcggcgagggaaactatgcatggtttgcatactgggggcaggggaccaccgtgactcactgctaggatctgcgattcttcggggtcgcccttattactacca

SEQ ID NO: 556 - DNA, 187 bp, Artificial
  Synthesized sequence
ccctttaatcagatgcgtcggtcctagtgaggaataccgggcagtgggcaggggaccaccgtgactgtgtcctcaggggggagcgctggtagcggttccagcggcggggccagcggttccgggggggacatcgagctcactcactgctaggatctgcgattcttcggggtcgcccttattactacca

SEQ ID NO: 557 - DNA, 187 bp, Artificial
  Synthesized sequence
ccctttaatcagatgcgtcggtcctagtgaggaataccgggcagtggggggggacatcgagctcactcagtctcctgcaagcctgtcagcatcagttggggagacagttaccatcacctgccaggcatccgaaaatatatacactgctaggatctgcgattcttcggggtcgcccttattactacca

SEQ ID NO: 558 - DNA, 187 bp, Artificial
  Synthesized sequence
ccctttaatcagatgcgtcggtcctagtgaggaataccgggcagtgctgccaggcatccgaaaatatatacagctacctcgcatggcatcagcaaaagcagggtaaaagccctcagctcctggtttataatgctaaaaccccactgctaggatctgcgattcttcggggtcgcccttattactacca

SEQ ID NO: 559 - DNA, 187 bp, Artificial
  Synthesized sequence
ccctttaatcagatgcgtcggtcctagtgaggaataccgggcagtgcagctcctggtttataatgctaaaaccctggctggaggcgtctcttcaagatttagcgggagcggctccgggacccacttctcactgaaaataaacactgctaggatctgcgattcttcggggtcgcccttattactacca

SEQ ID NO: 560 - DNA, 187 bp, Artificial
  Synthesized sequence
ccctttaatcagatgcgtcggtcctagtgaggaataccgggcagtggggacccacttctcactgaaaataaagtccctgcaaccagaggattttggtatttactattgtcagcaccactacggcatactcccaaccttcggcactgctaggatctgcgattcttcggggtcgcccttattactacca

SEQ ID NO: 561 - DNA, 168 bp, Artificial
  Synthesized sequence
ccctttaatcagatgcgtcggtcctagtgaggaataccgggcagtgtacggcatactcccaaccttcggagggggaactaagctggaaatcaaggggccctgcatgggtctgtctattgtttcactgctaggatctgcgattcttcggggtcgcccttattactacca

SEQ ID NO: 562 - DNA, 187 bp, Artificial
  Synthesized sequence
ccctttaatcagatgcgtcgttagataggtgtgtaggcgcgcagtgttccattgatagattcgctcgcggcccagccggccaggcgccaggttaccctgcgcgagagcgggcctgctctggtgaaacccactcagaccctgcactgcgtcagctagtacgcaccttaggtcgcccttattactacca

SEQ ID NO: 563 - DNA, 183 bp, Artificial
  Synthesized sequence
ccctttaatcagatgcgtcgttagataggtgtgtaggcgcgcagtgtggtgaaacccactcagaccctgactctgacctgcacattctctggcttttccctctctactgccggaatgtcagtgggatggatccgccacactgcgtcagctagtacgcaccttaggtcgcccttattactacca

SEQ ID NO: 564 - DNA, 191 bp, Artificial
  Synthesized sequence
ccctttaatcagatgcgtcgttagataggtgtgtaggcgcgcagtgtcagtgggatggatccgccagcctcctggcaaagctctggagtggctcgctgatatttggtgggacgataaaaagcattataatccatctctgaaggaccactgcgtcagctagtacgcaccttaggtcgcccttattactacca

SEQ ID NO: 565 - DNA, 187 bp, Artificial
  Synthesized sequence
ccctttaatcagatgcgtcgttagataggtgtgtaggcgcgcagtgaaagcattataatccatctctgaaggaccgcctcaccatcagcaaggacactagcaagaatcaggtggttctcaaggtgaccaatatggacccagcactgcgtcagctagtacgcaccttaggtcgcccttattactacca

SEQ ID NO: 566 - DNA, 186 bp, Artificial
  Synthesized sequence
ccctttaatcagatgcgtcgttagataggtgtgtaggcgcgcagtgtcaaggtgaccaatatggacccagctgataccgctacctactactgtgccagggacatgatcttcaacttctattttgacgtgtggggtcagggcactgcgtcagctagtacgcaccttaggtcgcccttattactacca

SEQ ID NO: 567 - DNA, 153 bp, Artificial
  Synthesized sequence
gcgcgcagtgtattttgacgtgtggggtcagggcaccaccgtcaccgttagctctgggggagccggtagcggggccgggagcgggagcagcggcgcaggctctggagcactgcgtcagctagtacgcaccttaggtcgcccttattactacca

SEQ ID NO: 568 - DNA, 184 bp, Artificial
  Synthesized sequence
ccctttaatcagatgcgtcgttagataggtgtgtaggcgcgcagtggcggcgcaggctctggagatatacagatgactcagagcccctctaccctgtctgcttccgtgggcgaccgggtcaccatcacatgctccgcccactgcgtcagctagtacgcaccttaggtcgcccttattactacca

SEQ ID NO: 569 - DNA, 187 bp, Artificial
  Synthesized sequence
ccctttaatcagatgcgtcgttagataggtgtgtaggcgcgcagtggtcaccatcacatgctccgcctctagccgcgtcggttatatgcattggtaccagcagaagcccggcaaggcacccaaactcctcatttatgacaccactgcgtcagctagtacgcaccttaggtcgcccttattactacca

SEQ ID NO: 570 - DNA, 187 bp, Artificial
  Synthesized sequence
ccctttaatcagatgcgtcgttagataggtgtgtaggcgcgcagtggcacccaaactcctcatttatgacacctccaagctggcctctggagttccctctcggttttccggaagcggtagcggcaccgagttcacactgaccactgcgtcagctagtacgcaccttaggtcgcccttattactacca

SEQ ID NO: 571 - DNA, 187 bp, Artificial
  Synthesized sequence
ccctttaatcagatgcgtcgttagataggtgtgtaggcgcgcagtgcggcaccgagttcacactgaccatctcctctctccagccagatgatttcgccacatattattgcttccagggcagcgggtatccttttacatttgcactgcgtcagctagtacgcaccttaggtcgcccttattactacca

SEQ ID NO: 572 - DNA, 170 bp, Artificial
  Synthesized sequence
ccctttaatcagatgcgtcgttagataggtgtgtaggcgcgcagtggcagcgggtatccttttacatttggtgggggaactaaagtggagatcaaagggcccctcctatgctagctcgactcttcactgcgtcagctagtacgcaccttaggtcgcccttattactacca

SEQ ID NO: 573 - DNA, 181 bp, Artificial
  Synthesized sequence
ccctttaatcagatgcgtcgttccgtttatgctttccagcgcagtgttttttctactttccggcttgcggcccagccggccaggcgcgaggtgcaactccagcagtctggtcccgagctggagaagcccggcgcccactgcctcgctctaaactccaaggaggtcgcccttattactacca

SEQ ID NO: 574 - DNA, 187 bp, Artificial
  Synthesized sequence
ccctttaatcagatgcgtcgttccgtttatgctttccagcgcagtgctggagaagcccggcgccagcgtgaagctgtcatgtaaagccagcgggtactcattcactggctataatatgaactgggtgaaacagtcacatggcactgcctcgctctaaactccaaggaggtcgcccttattactacca

SEQ ID NO: 575 - DNA, 191 bp, Artificial
  Synthesized sequence
ccctttaatcagatgcgtcgttccgtttatgctttccagcgcagtggaactgggtgaaacagtcacatggtaagagcctggaatggatcggccatattgacccctattacggtgacacttcttataaccaaaaattcaggggtaacactgcctcgctctaaactccaaggaggtcgcccttattactacca

SEQ ID NO: 576 - DNA, 187 bp, Artificial
  Synthesized sequence
ccctttaatcagatgcgtcgttccgtttatgctttccagcgcagtgcttcttataaccaaaaattcaggggtaaggccaccctgaccgtggacaaatctagcagcacagcctatatgcagctcaaatccctgacatcagaacactgcctcgctctaaactccaaggaggtcgcccttattactacca

SEQ ID NO: 577 - DNA, 184 bp, Artificial
  Synthesized sequence
ccctttaatcagatgcgtcgttccgtttatgctttccagcgcagtgcagctcaaatccctgacatcagaagacagcgctgtttattattgtgtgaaaggcgggtactacggtcattggtatttcgacgtgtggggcgccactgcctcgctctaaactccaaggaggtcgcccttattactacca

SEQ ID NO: 578 - DNA, 187 bp, Artificial
  Synthesized sequence
ccctttaatcagatgcgtcgttccgtttatgctttccagcgcagtggtatttcgacgtgtggggcgccgggaccactgtgactgtgtcctctggcggatctggcggctctggcggggcctccggagccggatctgggggcgcactgcctcgctctaaactccaaggaggtcgcccttattactacca

SEQ ID NO: 579 - DNA, 187 bp, Artificial
  Synthesized sequence
ccctttaatcagatgcgtcgttccgtttatgctttccagcgcagtgggagccggatctgggggcggcgacattcagatgacacaatcaccatcttctctgtccgcttccctgggtgagcgcgtctccctcacatgccgggccactgcctcgctctaaactccaaggaggtcgcccttattactacca

SEQ ID NO: 580 - DNA, 187 bp, Artificial
  Synthesized sequence
ccctttaatcagatgcgtcgttccgtttatgctttccagcgcagtggtctccctcacatgccgggcttctcaggacataggcagctccctcaactggctgcaacagggtccagacggtactatcaagcggctcatttatgccactgcctcgctctaaactccaaggaggtcgcccttattactacca

SEQ ID NO: 581 - DNA, 186 bp, Artificial
  Synthesized sequence
ccctttaatcagatgcgtcgttccgtttatgctttccagcgcagtggtactatcaagcggctcatttatgctacctctagcctggattcaggcgtgcccaaaaggttttctggatctcggtccggctcagactattccctcactgcctcgctctaaactccaaggaggtcgcccttattactacca

SEQ ID NO: 582 - DNA, 185 bp, Artificial
  Synthesized sequence
ccctttaatcagatgcgtcgttccgtttatgctttccagcgcagtgcggtccggctcagactattccctcactatttcttctctcgaaagcgaggatttcgtggactattactgtctgcagtacgtgagctcacctcctcactgcctcgctctaaactccaaggaggtcgcccttattactacca

SEQ ID NO: 583 - DNA, 175 bp, Artificial
  Synthesized sequence
ccctttaatcagatgcgtcgttccgtttatgctttccagcgcagtggcagtacgtgagctcacctcctactttcggggcaggcaccaaactcgaactgaaggggcccatggtaagaagctcccacaattcactgcctcgctctaaactccaaggaggtcgcccttattactacca

SEQ ID NO: 584 - DNA, 187 bp, Artificial
  Synthesized sequence
ccctttaatcagatgcgtcggtatagtttgtgcggtggtcgcagtgttatgactattggggtcgtaccggcccagccggccaggcgcgaagttcagctggtccagtcaggaggaggggtcgaacggcccggcggatctctgcactgccgaaggtgtaggggattgatggtcgcccttattactacca

SEQ ID NO: 585 - DNA, 188 bp, Artificial
  Synthesized sequence
ccctttaatcagatgcgtcggtatagtttgtgcggtggtcgcagtgcggcccggcggatctctgcggctgtcctgcgccgccagcggcttcacattcgatgattacggtatgagctgggttagacaagctccagggaaaggacactgccgaaggtgtaggggattgatggtcgcccttattactacca

SEQ ID NO: 586 - DNA, 187 bp, Artificial
  Synthesized sequence
ccctttaatcagatgcgtcggtatagtttgtgcggtggtcgcagtgggttagacaagctccagggaaaggactggagtgggtgtccggcatcaattggaacggtggcagcacaggctatgctgatagcgtcaagggcagagcactgccgaaggtgtaggggattgatggtcgcccttattactacca

SEQ ID NO: 587 - DNA, 186 bp, Artificial
  Synthesized sequence
ccctttaatcagatgcgtcggtatagtttgtgcggtggtcgcagtggctgatagcgtcaagggcagagttacaatcagcagagacaatgccaagaactctctgtatctccagatgaactccctgagggctgaagataccgcactgccgaaggtgtaggggattgatggtcgcccttattactacca

SEQ ID NO: 588 - DNA, 187 bp, Artificial
  Synthesized sequence
ccctttaatcagatgcgtcggtatagtttgtgcggtggtcgcagtgctccctgagggctgaagataccgcagtctattattgcgccaaaattctgggagccggaagaggatggtactttgatctctgggggaaaggaactacactgccgaaggtgtaggggattgatggtcgcccttattactacca

SEQ ID NO: 589 - DNA, 187 bp, Artificial
  Synthesized sequence
ccctttaatcagatgcgtcggtatagtttgtgcggtggtcgcagtgtgatctctgggggaaaggaactacagtcacagtgtctgggggcagcgcaggcagcggctccagcggcggggcttccggatcaggagggtcctccgcactgccgaaggtgtaggggattgatggtcgcccttattactacca

SEQ ID NO: 590 - DNA, 187 bp, Artificial
  Synthesized sequence
ccctttaatcagatgcgtcggtatagtttgtgcggtggtcgcagtgtccggatcaggagggtcctccgagctcactcaggacccagctgtgtctgtcgccctcgggcagactgtgcggatcacttgtcagggagattccctcactgccgaaggtgtaggggattgatggtcgcccttattactacca

SEQ ID NO: 591 - DNA, 187 bp, Artificial
  Synthesized sequence
ccctttaatcagatgcgtcggtatagtttgtgcggtggtcgcagtggatcacttgtcagggagattccctccgctcctattatgcctcctggtaccagcagaaacctggccaggcccccgtgctggtcatctacggcaaaacactgccgaaggtgtaggggattgatggtcgcccttattactacca

SEQ ID NO: 592 - DNA, 187 bp, Artificial
  Synthesized sequence
ccctttaatcagatgcgtcggtatagtttgtgcggtggtcgcagtggtgctggtcatctacggcaaaaataatcgcccatcaggcattcccgaccggtttagcggatcttcttccgggaatactgcctctctgacaattaccactgccgaaggtgtaggggattgatggtcgcccttattactacca

SEQ ID NO: 593 - DNA, 187 bp, Artificial
  Synthesized sequence
ccctttaatcagatgcgtcggtatagtttgtgcggtggtcgcagtggggaatactgcctctctgacaattactggtgcccaagctgaggatgaggccgattactactgtaacagccgcgacagctcaggaaaccacgtggtcactgccgaaggtgtaggggattgatggtcgcccttattactacca

SEQ ID NO: 594 - DNA, 173 bp, Artificial
  Synthesized sequence
ccctttaatcagatgcgtcggtatagtttgtgcggtggtcgcagtgacagctcaggaaaccacgtggtgttcgggggcggaactaagctcaccgtgctggggcccctatggtcattcccgtacgattcactgccgaaggtgtaggggattgatggtcgcccttattactacca

SEQ ID NO: 595 - DNA, 185 bp, Artificial
  Synthesized sequence
ccctttaatcagatgcgtcgtcagcctttcattgattgcggcagtgtttcgacaatagttgagcccttggcccagccggccaggcgccaggtgcagctgcaacaatccggccccgaggttgtgaaaccaggcgcctctgcactgccgagctacggtatcaaggaaggtcgcccttattactacca

SEQ ID NO: 596 - DNA, 185 bp, Artificial
  Synthesized sequence
ccctttaatcagatgcgtcgtcagcctttcattgattgcggcagtgtgtgaaaccaggcgcctctgtgaagatgtcttgcaaggcctcaggctatacattcaccagctatgtgattcactgggtgcgccagaaaccaggcactgccgagctacggtatcaaggaaggtcgcccttattactacca

SEQ ID NO: 597 - DNA, 189 bp, Artificial
  Synthetic sequence
ccctttaatcagatgcgtcgtcagcctttcattgattgcggcagtgtgggtgcgccagaaaccaggacagggtctcgattggattggctatattaacccttacaatgatggtacagactatgacgagaagtttaaaggcaaggcactgccgagctacggtatcaaggaaggtcgcccttattactacca

SEQ ID NO: 598 - DNA, 189 bp, Artificial
  Synthesized sequence
ccctttaatcagatgcgtcgtcagcctttcattgattgcggcagtgtatgacgagaagtttaaaggcaaggccacactgacaagcgatacctctactagcaccgcctatatggagctcagctccctccggtcagaagacaccgcactgccgagctacggtatcaaggaaggtcgcccttattactacca

SEQ ID NO: 599 - DNA, 189 bp, Artificial
  Synthesized sequence
ccctttaatcagatgcgtcgtcagcctttcattgattgcggcagtgtccctccggtcagaagacaccgctgtgtattattgtgccagagaaaaagataattatgctacaggcgcttggttcgcctactggggacaggggactccactgccgagctacggtatcaaggaaggtcgcccttattactacca

SEQ ID NO: 600 - DNA, 190 bp, Artificial
  Synthesized sequence
ccctttaatcagatgcgtcgtcagcctttcattgattgcggcagtggcctactggggacaggggactctcgtgactgtgtcaagcggtggagccgggtccggcgccggctctggttccagcggggccggttccggggacattgtcactgccgagctacggtatcaaggaaggtcgcccttattactacca

SEQ ID NO: 601 - DNA, 191 bp, Artificial
  Synthesized sequence
ccctttaatcagatgcgtcgtcagcctttcattgattgcggcagtggccggttccggggacattgtgatgacccagtctccagatagcctggctgtgtctctgggcgagagggtgacaatgaattgtaagtcctcacaaagcctccactgccgagctacggtatcaaggaaggtcgcccttattactacca

SEQ ID NO: 602 - DNA, 189 bp, Artificial
  Synthesized sequence
ccctttaatcagatgcgtcgtcagcctttcattgattgcggcagtgtgaattgtaagtcctcacaaagcctcctgtattctaccaatcagaagaactacctggcttggtatcaacagaagccaggccaatctcccaagctcctcactgccgagctacggtatcaaggaaggtcgcccttattactacca

SEQ ID NO: 603 - DNA, 189 bp, Artificial
  Synthesized sequence
ccctttaatcagatgcgtcgtcagcctttcattgattgcggcagtgcaggccaatctcccaagctcctcatttattgggcttccacaagggagtccggcgtgccagaccggtttagcggatccggctccggcactgatttcaccactgccgagctacggtatcaaggaaggtcgcccttattactacca

SEQ ID NO: 604 - DNA, 189 bp, Artificial
  Synthesized sequence
ccctttaatcagatgcgtcgtcagcctttcattgattgcggcagtgcggctccggcactgatttcaccctcaccatcagctccgttcaagccgaagatgtggccgtctactactgccagcaatattattcctatcgcacctttcactgccgagctacggtatcaaggaaggtcgcccttattactacca

SEQ ID NO: 605 - DNA, 174 bp, Artificial
  Synthesized sequence
ccctttaatcagatgcgtcgtcagcctttcattgattgcggcagtgcagcaatattattcctatcgcacctttggcggagggactaaactggagattaaggggccctaatcggctacgttgtgtctttcactgccgagctacggtatcaaggaaggtcgcccttattactacca

SEQ ID NO: 606 - DNA, 185 bp, Artificial
  Synthesized sequence
ccctttaatcagatgcgtcgagggtcgtggttaaaggtacgcagtgttgagccatgtgaaatgtgtgtggcccagccggccaggcgcgagatccaactccagcagtctggacctgagctggtgaagccaggtgcctctgcactgcctaacgaccggaaagaaacgggtcgcccttattactacca

SEQ ID NO: 607 - DNA, 189 bp, Artificial
  Synthesized sequence
ccctttaatcagatgcgtcgagggtcgtggttaaaggtacgcagtgggtgaagccaggtgcctctgtgaaggtgtcatgcaaagcttccggctatgcatttacatcttacaatatgtattgggtgaagcaatcacatggcaagcactgcctaacgaccggaaagaaacgggtcgcccttattactacca

SEQ ID NO: 608 - DNA, 189 bp, Artificial
  Synthesized sequence
ccctttaatcagatgcgtcgagggtcgtggttaaaggtacgcagtggggtgaagcaatcacatggcaagagcctggagtggattggctatattgatccatataatggcgtgacctcttacaaccagaaattcaaggggaaggccactgcctaacgaccggaaagaaacgggtcgcccttattactacca

SEQ ID NO: 609 - DNA, 189 bp, Artificial
  Synthesized sequence
ccctttaatcagatgcgtcgagggtcgtggttaaaggtacgcagtgcaaccagaaattcaaggggaaggctaccctcacagttgacaagtcttcttctactgcctatatgcacctcaattcactgacatctgaggactctgcccactgcctaacgaccggaaagaaacgggtcgcccttattactacca

SEQ ID NO: 610 - DNA, 186 bp, Artificial
  Synthesized sequence
ccctttaatcagatgcgtcgagggtcgtggttaaaggtacgcagtgtcactgacatctgaggactctgccgtgtattattgcgctaggggtggaggaagcatctactatgccatggactattggggacaagggaccagcgcactgcctaacgaccggaaagaaacgggtcgcccttattactacca

SEQ ID NO: 611 - DNA, 189 bp, Artificial
  Synthesized sequence
ccctttaatcagatgcgtcgagggtcgtggttaaaggtacgcagtgattggggacaagggaccagcgtgactgtctcaagcggcggctctggcggcagcggcggcgccagcggcgcaggctccggggggggagatattgtgatcactgcctaacgaccggaaagaaacgggtcgcccttattactacca

SEQ ID NO: 612 - DNA, 189 bp, Artificial
  Synthesized sequence
ccctttaatcagatgcgtcgagggtcgtggttaaaggtacgcagtgccggggggggagatattgtgatgacacaggccgcaccttccgtgcctgtgacccctggggagtcagtgagcatcagctgccgctcctccaagtccctcactgcctaacgaccggaaagaaacgggtcgcccttattactacca

SEQ ID NO: 613 - DNA, 189 bp, Artificial
  Synthesized sequence
ccctttaatcagatgcgtcgagggtcgtggttaaaggtacgcagtgtgccgctcctccaagtccctgctgcattccaatggcaatacctatctctattggttcctccagagaccaggacaatccccacagctgctgatctacacactgcctaacgaccggaaagaaacgggtcgcccttattactacca

SEQ ID NO: 614 - DNA, 187 bp, Artificial
  Synthesized sequence
ccctttaatcagatgcgtcgagggtcgtggttaaaggtacgcagtgtccccacagctgctgatctacagaatgtccaacctcgcatctggagtccctgaccggttctcaggcagcggtagcggcaccgcatttactctgcgcactgcctaacgaccggaaagaaacgggtcgcccttattactacca

SEQ ID NO: 615 - DNA, 189 bp, Artificial
  Synthesized sequence
ccctttaatcagatgcgtcgagggtcgtggttaaaggtacgcagtggcggcaccgcatttactctgcggatttctagggtggaggccgaagatgtgggtgtgtactactgtatgcaacacctggagtatcccctgacttttggcactgcctaacgaccggaaagaaacgggtcgcccttattactacca

SEQ ID NO: 616 - DNA, 169 bp, Artificial
  Synthesized sequence
ccctttaatcagatgcgtcgagggtcgtggttaaaggtacgcagtgcctggagtatcccctgacttttggagccggaaccaagctcgaactgaaggggccctgactcgatcctttagtccgttcactgcctaacgaccggaaagaaacgggtcgcccttattactacca

SEQ ID NO: 617 - DNA, 187 bp, Artificial
  Synthesized sequence
ccctttaatcagatgcgtcgtgcaagtgtacaaatccagcgcagtgttcgtatacgtaagggttccgaggcccagccggccaggcgccaggtgcaactcgtggaatctggaggcggcgtcgtgcagcccgggaggtctctgcactgctaggaaagggatcaccgttcggtcgcccttattactacca

SEQ ID NO: 618 - DNA, 187 bp, Artificial
  Synthesized sequence
ccctttaatcagatgcgtcgtgcaagtgtacaaatccagcgcagtggcagcccgggaggtctctgcggctgtcatgtgcagcttcaggcttcactttcagcgtctatggtatgaactgggtgagacaggcacctggaaaagcactgctaggaaagggatcaccgttcggtcgcccttattactacca

SEQ ID NO: 619 - DNA, 187 bp, Artificial
  Synthesized sequence
ccctttaatcagatgcgtcgtgcaagtgtacaaatccagcgcagtggtgagacaggcacctggaaaaggactcgaatgggtggccatcatctggtacgacggcgacaaccaatactacgccgactccgtcaaggggagattcactgctaggaaagggatcaccgttcggtcgcccttattactacca

SEQ ID NO: 620 - DNA, 185 bp, Artificial
  Synthesized sequence
ccctttaatcagatgcgtcgtgcaagtgtacaaatccagcgcagtgccgactccgtcaaggggagattcacaatttcacgcgataactccaaaaatacactgtacctccagatgaacggcctgagagctgaggacacagcactgctaggaaagggatcaccgttcggtcgcccttattactacca

SEQ ID NO: 621 - DNA, 187 bp, Artificial
  Synthesized sequence
ccctttaatcagatgcgtcgtgcaagtgtacaaatccagcgcagtgggcctgagagctgaggacacagccgtttattactgtgccagggacctccggaccggacccttcgactattggggacaggggacactggtcacagtcactgctaggaaagggatcaccgttcggtcgcccttattactacca

SEQ ID NO: 622 - DNA, 187 bp, Artificial
  Synthesized sequence
ccctttaatcagatgcgtcgtgcaagtgtacaaatccagcgcagtgacaggggacactggtcacagtgtcaagcgcttccggagggtctgcagggtccggatccagcgggggggcttcagggagcggaggggagatcgttccactgctaggaaagggatcaccgttcggtcgcccttattactacca

SEQ ID NO: 623 - DNA, 187 bp, Artificial
  Synthesized sequence
ccctttaatcagatgcgtcgtgcaagtgtacaaatccagcgcagtggagcggaggggagatcgttctgactcagtctccagactttcagtctgtcacaccaaaggaaaaggtcaccatcacttgccgggcctcacaatccacactgctaggaaagggatcaccgttcggtcgcccttattactacca

SEQ ID NO: 624 - DNA, 189 bp, Artificial
  Synthesized sequence
ccctttaatcagatgcgtcgtgcaagtgtacaaatccagcgcagtgtgctcatcaagtacgcttcacagtctttcagcggcgtcccatccaggttctccggctccggttccggcacagacttcactctgaccatcaatagcctcactgctaggaaagggatcaccgttcggtcgcccttattactacca

SEQ ID NO: 625 - DNA, 187 bp, Artificial
  Synthesized sequence
ccctttaatcagatgcgtcgtgcaagtgtacaaatccagcgcagtggacttcactctgaccatcaatagcctcgaagctgaagacgctgctgcttattactgtcaccaaagcagctctctgccctttacttttggtcctggcactgctaggaaagggatcaccgttcggtcgcccttattactacca

SEQ ID NO: 626 - DNA, 163 bp, Artificial
  Synthesized sequence
ccctttaatcagatgcgtcgtgcaagtgtacaaatccagcgcagtgtctgccctttacttttggtcctggcacaaaggtggacattaaggggcccacgctttgtgttatccgatgttcactgctaggaaagggatcaccgttcggtcgcccttattactacca

SEQ ID NO: 627 - DNA, 192 bp, Artificial
  Synthesized sequence
ccctttaatcagatgcgtcgcttaaggtttgcccattcccgcagtgttttatgatgtccggatacccgggcccagccggccaggcgccaggtgcagctggtggaaagcggtggcggtgtcgtgcagcccggccgcagcctgagactcactgcacaccgtggaagctataacaggtcgcccttattactacca

SEQ ID NO: 628 - DNA, 193 bp, Artificial
  Synthesized sequence
ccctttaatcagatgcgtcgcttaaggtttgcccattcccgcagtgcggccgcagcctgagactctcctgcgctgcatcaggttttacattttctagctacgatatgtcttgggtccggcaggcaccaggaaaggggctggagtgggcactgcacaccgtggaagctataacaggtcgcccttattactacca

SEQ ID NO: 629 - DNA, 193 bp, Artificial
  Synthesized sequence
ccctttaatcagatgcgtcgcttaaggtttgcccattcccgcagtgcaggaaaggggctggagtgggtggctaaagtttcttccggaggggggagcacctactatctcgacactgttcagggccggttcactatatcccgggacaatcactgcacaccgtggaagctataacaggtcgcccttattactacca

SEQ ID NO: 630 - DNA, 193 bp, Artificial
  Synthesized sequence
ccctttaatcagatgcgtcgcttaaggtttgcccattcccgcagtgcggttcactatatcccgggacaattctaagaatacactgtacctgcagatgaattctctgagggcagaagataccgctgtgtactattgtgcacggcatctcactgcacaccgtggaagctataacaggtcgcccttattactacca

SEQ ID NO: 631 - DNA, 193 bp, Artificial
  Synthesized sequence
ccctttaatcagatgcgtcgcttaaggtttgcccattcccgcagtgtgtgtactattgtgcacggcatctgcacggatccttcgcttcctggggacagggcactactgtcaccgtttctagcggcggtgctggatctggagctggatcactgcacaccgtggaagctataacaggtcgcccttattactacca

SEQ ID NO: 632 - DNA, 190 bp, Artificial
  Synthesized sequence
ccctttaatcagatgcgtcgcttaaggtttgcccattcccgcagtggtgctggatctggagctggatcagggtcctctggagctggctcaggtgagatcgtgctgacccaaagccctgctaccctgagcctctccccaggagagcactgcacaccgtggaagctataacaggtcgcccttattactacca

SEQ ID NO: 633 - DNA, 187 bp, Artificial
  Synthesized sequence
ccctttaatcagatgcgtcgcttaaggtttgcccattcccgcagtgctgagcctctccccaggagagcgggcaacactgtcttgtcaggcatctcaatcaattagcaacttcctgcattggtaccaacagcggccaggccacactgcacaccgtggaagctataacaggtcgcccttattactacca

SEQ ID NO: 634 - DNA, 193 bp, Artificial
  Synthesized sequence
ccctttaatcagatgcgtcgcttaaggtttgcccattcccgcagtgccaacagcggccaggccaagcccctaggctgctcattagatacaggtcccaatcaattagcggaataccagccaggttttccggctctggatccggtaccgcactgcacaccgtggaagctataacaggtcgcccttattactacca

SEQ ID NO: 635 - DNA, 193 bp, Artificial
  Synthesized sequence
ccctttaatcagatgcgtcgcttaaggtttgcccattcccgcagtgccggctctggatccggtaccgacttcaccctcaccatctcttccctggaacccgaagacttcgccgtgtattactgtcagcagtctgggtcttggcctctgcactgcacaccgtggaagctataacaggtcgcccttattactacca

SEQ ID NO: 636 - DNA, 174 bp, Artificial
  Synthesized sequence
ccctttaatcagatgcgtcgcttaaggtttgcccattcccgcagtgcagtctgggtcttggcctctgacattcggaggtggaactaaagtggaaatcaaagggcccaccacggtggagtatacatcttcactgcacaccgtggaagctataacaggtcgcccttattactacca

SEQ ID NO: 637 - DNA, 188 bp, Artificial
  Synthesized sequence
ccctttaatcagatgcgtcgtggttcgttagtcgatctccgcagtgtttcttagaaatccacgggtccggcccagccggccaggcgcgaagtgcagctgctggaaagcggcggcgggctggtccagcccggcggatccctgacactgcgacccagtaaaatcccgtctggtcgcccttattactacca

SEQ ID NO: 638 - DNA, 188 bp, Artificial
  Synthesized sequence
ccctttaatcagatgcgtcgtggttcgttagtcgatctccgcagtgagcccggcggatccctgagactgtcatgtgccgccagcggtttcacttttagctcatttccaatggcctgggttcggcaggcaccaggaaaaggcccactgcgacccagtaaaatcccgtctggtcgcccttattactacca

SEQ ID NO: 639 - DNA, 188 bp, Artificial
  Synthesized sequence
ccctttaatcagatgcgtcgtggttcgttagtcgatctccgcagtgggcaggcaccaggaaaaggcctcgaatgggtgtccacaatatcaacttctggcggtagaacatactatagggactccgtgaagggcagatttaccacactgcgacccagtaaaatcccgtctggtcgcccttattactacca

SEQ ID NO: 640 - DNA, 189 bp, Artificial
  Synthesized sequence
ccctttaatcagatgcgtcgtggttcgttagtcgatctccgcagtgactccgtgaagggcagatttaccatttcccgggataatagcaagaatacactgtatctgcagatgaattcactgagggctgaagatacagccgtgtacactgcgacccagtaaaatcccgtctggtcgcccttattactacca

SEQ ID NO: 641 - DNA, 188 bp, Artificial
  Synthesized sequence
ccctttaatcagatgcgtcgtggttcgttagtcgatctccgcagtggggctgaagatacagccgtgtattattgcgccaaatttcgccagtattctggcggctttgactactggggacagggcactctcgtcacagtgagctcactgcgacccagtaaaatcccgtctggtcgcccttattactacca

SEQ ID NO: 642 - DNA, 187 bp, Artificial
  Synthesized sequence
ccctttaatcagatgcgtcgtgcaagtgtacaaatccagcgcagtgttgccgggcctcacaatccatcggttctagcctgcactggtatcagcagaaaccagaccagtcccccaagctgctcatcaagtacgcttcacagtcactgctaggaaagggatcaccgttcggtcgcccttattactacca

SEQ ID NO: 643 - DNA, 188 bp, Artificial
  Synthesized sequence
ccctttaatcagatgcgtcgtggttcgttagtcgatctccgcagtggggcactctcgtcacagtgagctctggcgggtccggaggctctggcggcgcctcaggcgcaggctccggaggcggcgacattcagctcactcaacccactgcgacccagtaaaatcccgtctggtcgcccttattactacca

SEQ ID NO: 644 - DNA, 188 bp, Artificial
  Synthesized sequence
ccctttaatcagatgcgtcgtggttcgttagtcgatctccgcagtgggcgacattcagctcactcaacccaacagcgtgtcaacttctctgggatccaccgtgaagctgtcctgtactctcagctctgggaatatcgaaaatcactgcgacccagtaaaatcccgtctggtcgcccttattactacca

SEQ ID NO: 645 - DNA, 190 bp, Artificial
  Synthesized sequence
ccctttaatcagatgcgtcgtggttcgttagtcgatctccgcagtgctcagctctgggaatatcgaaaataactacgtgcattggtaccagctctatgaggggcggagccccactaccatgatttatgacgacgataaacgccccactgcgacccagtaaaatcccgtctggtcgcccttattactacca

SEQ ID NO: 646 - DNA, 188 bp, Artificial
  Synthesized sequence
ccctttaatcagatgcgtcgtggttcgttagtcgatctccgcagtgatgatttatgacgacgataaacgccctgacggtgtgcctgatagattttctggcagcatcgatcggtctagcaatagcgcattcctgactatccatcactgcgacccagtaaaatcccgtctggtcgcccttattactacca

SEQ ID NO: 647 - DNA, 187 bp, Artificial
  Synthesized sequence
ccctttaatcagatgcgtcgtggttcgttagtcgatctccgcagtgaatagcgcattcctgactatccataatgtggcaatcgaggatgaggctatctacttctgtcactcctatgtgagctccttcaacgtcttcggtggcactgcgacccagtaaaatcccgtctggtcgcccttattactacca

SEQ ID NO: 648 - DNA, 165 bp, Artificial
  Synthesized sequence
ccctttaatcagatgcgtcgtggttcgttagtcgatctccgcagtgagctccttcaacgtcttcggtggcggcacaaaactgactgttctcgggcccggcaccaggtacatatctcattcactgcgacccagtaaaatcccgtctggtcgcccttattactacca

SEQ ID NO: 649 - DNA, 187 bp, Artificial
  Synthesized sequence
ccctttaatcagatgcgtcgtattttgtagagcgttcgcggcagtgttgaagggtggatcatcgtactggcccagccggccaggcgcgaagaacaggttgttgagtcagggggcggatttgtgcagcctggaggatctctgcactgccaagacttgcgaagcaaagaggtcgcccttattactacca

SEQ ID NO: 650 - DNA, 185 bp, Artificial
  Synthesized sequence
ccctttaatcagatgcgtcgtattttgtagagcgttcgcggcagtggtgcagcctggaggatctctgagactcagctgcgcagccagcggcttcaccttttcaccatactggatgcactgggtgagacaagctcctggccactgccaagacttgcgaagcaaagaggtcgcccttattactacca

SEQ ID NO: 651 - DNA, 187 bp, Artificial
  Synthesized sequence
ccctttaatcagatgcgtcgtattttgtagagcgttcgcggcagtgctgggtgagacaagctcctggcaagggactcgtctgggtgtcacggattaattctgacggatcaacatactacgcagactcagtcaaaggaaggtcactgccaagacttgcgaagcaaagaggtcgcccttattactacca

SEQ ID NO: 652 - DNA, 188 bp, Artificial
  Synthesized sequence
ccctttaatcagatgcgtcgtattttgtagagcgttcgcggcagtgacgcagactcagtcaaaggaaggtttaccatatccagagataacgctagaaacacactgtatctgcagatgaactcactcagagctgaggatacagcactgccaagacttgcgaagcaaagaggtcgcccttattactacca

SEQ ID NO: 653 - DNA, 182 bp, Artificial
  Synthesized sequence
ccctttaatcagatgcgtcgtattttgtagagcgttcgcggcagtgaactcactcagagctgaggatacagcagtttactactgtgcaagagaccggtattatggtcctgagatgtggggccagggcacaatggtgcactgccaagacttgcgaagcaaagaggtcgcccttattactacca

SEQ ID NO: 654 - DNA, 187 bp, Artificial
  Synthesized sequence
ccctttaatcagatgcgtcgtattttgtagagcgttcgcggcagtggggccagggcacaatggtgaccgttagctctggcggcgcaggctctggggctggatcaggaagctccggtgctggtagcggcgatgtggtgatgacactgccaagacttgcgaagcaaagaggtcgcccttattactacca

SEQ ID NO: 655 - DNA, 187 bp, Artificial
  Synthesized sequence
ccctttaatcagatgcgtcgtattttgtagagcgttcgcggcagtgtagcggcgatgtggtgatgacccagtctccactcagcctccccgttacactcgggcaacccgcctctatttcttgccgctcctcccaatccctcgcactgccaagacttgcgaagcaaagaggtcgcccttattactacca

SEQ ID NO: 656 - DNA, 187 bp, Artificial
  Synthesized sequence
ccctttaatcagatgcgtcgtattttgtagagcgttcgcggcagtggccgctcctcccaatccctcgtgtactctgacggcaatacatacctgaattggttccagcagagacctgggcagtcaccaaggagactcatttaccactgccaagacttgcgaagcaaagaggtcgcccttattactacca

SEQ ID NO: 657 - DNA, 187 bp, Artificial
  Synthesized sequence
ccctttaatcagatgcgtcgtattttgtagagcgttcgcggcagtgcagtcaccaaggagactcatttacaaggtgagcaatcgcgacagcggggtgcccgaccggttcagcggcagcggctcagggaccgattttaccctcactgccaagacttgcgaagcaaagaggtcgcccttattactacca

SEQ ID NO: 658 - DNA, 182 bp, Artificial
  Synthesized sequence
ccctttaatcagatgcgtcgtattttgtagagcgttcgcggcagtgcggctcagggaccgattttaccctcaagatttcaagggtggaagctgaagatgtgggagtctattattgtatgcagggcacccactggcccactgccaagacttgcgaagcaaagaggtcgcccttattactacca

SEQ ID NO: 659 - DNA, 176 bp, Artificial
  Synthesized sequence
ccctttaatcagatgcgtcgtattttgtagagcgttcgcggcagtgtgcagggcacccactggcccctgacatttggcggcgggacaaaggtcgagatcaaggggcccacaacgataggcccaagaatttcactgccaagacttgcgaagcaaagaggtcgcccttattactacca

SEQ ID NO: 660 - DNA, 187 bp, Artificial
  Synthesized sequence
ccctttaatcagatgcgtcgttctgtaagtttcgtcgggagcagtgttggctgttagttttagagccgggcccagccggccaggcgccaggtcgagctggtggagtctggcggggggctggtgcaacctgggggaagcctgcactgctagtgaggtgcggtgtttagggtcgcccttattactacca

SEQ ID NO: 661 - DNA, 187 bp, Artificial
  Synthesized sequence
ccctttaatcagatgcgtcgttctgtaagtttcgtcgggagcagtgtgcaacctgggggaagcctgaggctgtcctgcgctgcatcagggttcacattctctagctatgcaatgtcctgggtgaggcaggcccctggaaaacactgctagtgaggtgcggtgtttagggtcgcccttattactacca

SEQ ID NO: 662 - DNA, 187 bp, Artificial
  Synthesized sequence
ccctttaatcagatgcgtcgttctgtaagtttcgtcgggagcagtgaggcaggcccctggaaaaggactggagtgggtctctgcaatcaatgcctctggcacccgcacttattatgctgacagcgtcaaggggaggtttaccactgctagtgaggtgcggtgtttagggtcgcccttattactacca

SEQ ID NO: 663 - DNA, 187 bp, Artificial
  Synthesized sequence
ccctttaatcagatgcgtcgttctgtaagtttcgtcgggagcagtgcagcgtcaaggggaggtttactatttctagggataactctaaaaataccctgtacctccagatgaactcactcagggccgaggatactgcagtttcactgctagtgaggtgcggtgtttagggtcgcccttattactacca

SEQ ID NO: 664 - DNA, 185 bp, Artificial
  Synthesized sequence
ccctttaatcagatgcgtcgttctgtaagtttcgtcgggagcagtggggccgaggatactgcagtttactattgcgctaggggtaaaggtaacacccacaagccttacggatatgtgaggtacttcgacgtgtgggggccactgctagtgaggtgcggtgtttagggtcgcccttattactacca

SEQ ID NO: 665 - DNA, 182 bp, Artificial
  Synthesized sequence
ccctttaatcagatgcgtcgttctgtaagtttcgtcgggagcagtgaggtacttcgacgtgtgggggcagggaaccggtggctccggcggaagcgggggagcttccggggctggctctggtgggggcgacatcgtgcactgctagtgaggtgcggtgtttagggtcgcccttattactacca

SEQ ID NO: 666 - DNA, 187 bp, Artificial
  Synthesized sequence
ccctttaatcagatgcgtcgttctgtaagtttcgtcgggagcagtgtggtgggggcgacatcgtgctcacccagtccccagccactctgagcctgagccctggagaaagagcaacactgtcttgccgggcctcccagtccgcactgctagtgaggtgcggtgtttagggtcgcccttattactacca

SEQ ID NO: 667 - DNA, 187 bp, Artificial
  Synthesized sequence
ccctttaatcagatgcgtcgttctgtaagtttcgtcgggagcagtggccgggcctcccagtccgtttccagcagctacctggcctggtatcagcagaaaccaggccaggcaccaaggctcctgatctatggtgcctcttcccactgctagtgaggtgcggtgtttagggtcgcccttattactacca

SEQ ID NO: 668 - DNA, 187 bp, Artificial
  Synthesized sequence
ccctttaatcagatgcgtcgttctgtaagtttcgtcgggagcagtgctcctgatctatggtgcctcttccagagcaaccggcgtgcctgctcggttctccgggtccggctcagggaccgacttcacactgactatatcctccactgctagtgaggtgcggtgtttagggtcgcccttattactacca

SEQ ID NO: 669 - DNA, 187 bp, Artificial
  Synthesized sequence
ccctttaatcagatgcgtcgttctgtaagtttcgtcgggagcagtgaccgacttcacactgactatatcctccctggagccagaggactttgccacatactattgtctgcaaatctacaatatgcccattacctttggccacactgctagtgaggtgcggtgtttagggtcgcccttattactacca

SEQ ID NO: 670 - DNA, 166 bp, Artificial
  Synthesized sequence
ccctttaatcagatgcgtcgttctgtaagtttcgtcgggagcagtgcaatatgcccattacctttggccagggtaccaaagtcgagatcaaggggcccacgacggctgtatatggtttttcactgctagtgaggtgcggtgtttagggtcgcccttattactacca

SEQ ID NO: 671 - DNA, 185 bp, Artificial
  Synthesized sequence
ccctttaatcagatgcgtcgttgacgtacgtaggttctccgcagtgttagtggtgtagtggcttctacggcccagccggccaggcgccaggtccagctgcagcagtctggatccgagctcaaaaagcccggagccagcgcactgcgcgtcagtgtagttgtgttcggtcgcccttattactacca

SEQ ID NO: 672 - DNA, 187 bp, Artificial
  Synthesized sequence
ccctttaatcagatgcgtcgttgacgtacgtaggttctccgcagtgcaaaaagcccggagccagcgttaaggtttcctgcaaagcctctggctataccttcactaattacggtgtgaactggattaagcaggccccaggcccactgcgcgtcagtgtagttgtgttcggtcgcccttattactacca

SEQ ID NO: 673 - DNA, 188 bp, Artificial
  Synthesized sequence
ccctttaatcagatgcgtcgttgacgtacgtaggttctccgcagtgtggattaagcaggccccaggccaggggctccaatggatgggctggataaaccctaatactggagagcctactttcgacgatgatttcaaggggcgccactgcgcgtcagtgtagttgtgttcggtcgcccttattactacca

SEQ ID NO: 674 - DNA, 189 bp, Artificial
  Synthesized sequence
ccctttaatcagatgcgtcgttgacgtacgtaggttctccgcagtgtcgacgatgatttcaaggggcgcttcgccttctctctggatacctccgtgtcaactgcctacctccagatctcaagcctgaaagccgacgatactgccactgcgcgtcagtgtagttgtgttcggtcgcccttattactacca

SEQ ID NO: 675 - DNA, 189 bp, Artificial
  Synthesized sequence
ccctttaatcagatgcgtcgttgacgtacgtaggttctccgcagtgagcctgaaagccgacgatactgccgtgtacttctgttctaggtccagagggaagaacgaggcctggttcgcatactggggtcaggggacactggtgacactgcgcgtcagtgtagttgtgttcggtcgcccttattactacca

SEQ ID NO: 676 - DNA, 186 bp, Artificial
  Synthesized sequence
ccctttaatcagatgcgtcgttgacgtacgtaggttctccgcagtgggggtcaggggacactggtgactgtgagctctggaggatcagcagggtcagggtcttccggcggggctagcggctcagggggcgacattcagctcactgcgcgtcagtgtagttgtgttcggtcgcccttattactacca

SEQ ID NO: 677 - DNA, 187 bp, Artificial
  Synthesized sequence
ccctttaatcagatgcgtcgttgacgtacgtaggttctccgcagtgctcagggggcgacattcagctcacccaatcaccactgtctctgcccgtgaccctcggacagcccgcttcaatctcatgccggtcttctcagtcaccactgcgcgtcagtgtagttgtgttcggtcgcccttattactacca

SEQ ID NO: 678 - DNA, 185 bp, Artificial
  Synthesized sequence
ccctttaatcagatgcgtcgttgacgtacgtaggttctccgcagtgtcatgccggtcttctcagtcactcgtccatcggaacggcaacacttatctgcactggtttcaacagcggccaggccaatctccccgcctgctgcactgcgcgtcagtgtagttgtgttcggtcgcccttattactacca

SEQ ID NO: 679 - DNA, 189 bp, Artificial
  Synthesized sequence
ccctttaatcagatgcgtcgttgacgtacgtaggttctccgcagtggccaatctccccgcctgctgatttacactgtgagcaatcggttctcaggtgttcctgacagatttagcgggagcggtagcggcactgattttactctcactgcgcgtcagtgtagttgtgttcggtcgcccttattactacca

SEQ ID NO: 680 - DNA, 186 bp, Artificial
  Synthesized sequence
ccctttaatcagatgcgtcgttgacgtacgtaggttctccgcagtgcggtagcggcactgattttactctgaagatttcccgcgtcgaagccgaggacgtcggggtgtacttttgcagccagagctctcatgtgcccccccactgcgcgtcagtgtagttgtgttcggtcgcccttattactacca

SEQ ID NO: 681 - DNA, 174 bp, Artificial
  Synthesized sequence
ccctttaatcagatgcgtcgttgacgtacgtaggttctccgcagtgcagagctctcatgtgccccccaccttcggcgcagggacacgcctggaaattaaggggccccatcgggtgggatttagctattcactgcgcgtcagtgtagttgtgttcggtcgcccttattactacca

SEQ ID NO: 682 - DNA, 187 bp, Artificial
  Synthesized sequence
ccctttaatcagatgcgtcggagatgagtagacgagtggggcagtgttctcagagggagttcaactgtggcccagccggccaggcgccaggtgcagctgcagcaatctggcgccgaagtgaaaaaaccaggttcctccgtccactgctaatgcgagtcagtgaccatggtcgcccttattactacca

SEQ ID NO: 683 - DNA, 187 bp, Artificial
  Synthesized sequence
ccctttaatcagatgcgtcggagatgagtagacgagtggggcagtggtgaaaaaaccaggttcctccgtcaaggtgagctgcaaggcctccggctacacctttacctcatacaacatgcactgggtgaaacaagctcctggcactgctaatgcgagtcagtgaccatggtcgcccttattactacca

SEQ ID NO: 684 - DNA, 187 bp, Artificial
  Synthesized sequence
ccctttaatcagatgcgtcggagatgagtagacgagtggggcagtgcactgggtgaaacaagctcctggtcagggcctggagtggattggcgcaatctatcccgggaatggcgacacttcttataaccaaaagttcaaaggcactgctaatgcgagtcagtgaccatggtcgcccttattactacca

SEQ ID NO: 685 - DNA, 186 bp, Artificial
  Synthesized sequence
ccctttaatcagatgcgtcggagatgagtagacgagtggggcagtgcgacacttcttataaccaaaagttcaaaggaaaggccacactcacagccgacgaaagcaccaatactgcctacatggagctgtctagcctccgccactgctaatgcgagtcagtgaccatggtcgcccttattactacca

SEQ ID NO: 686 - DNA, 187 bp, Artificial
  Synthesized sequence
ccctttaatcagatgcgtcggagatgagtagacgagtggggcagtgacatggagctgtctagcctccgctctgaggatactgccttctactactgtgctcggtccacttactacgggggggattggtacttcgatgtgtggcactgctaatgcgagtcagtgaccatggtcgcccttattactacca

SEQ ID NO: 687 - DNA, 181 bp, Artificial
  Synthesized sequence
ccctttaatcagatgcgtcggagatgagtagacgagtggggcagtgggggattggtacttcgatgtgtgggggcaaggcactactgtcacagtttcttctgggggggccgggagcggggccggaagcggcagctccactgctaatgcgagtcagtgaccatggtcgcccttattactacca

SEQ ID NO: 688 - DNA, 187 bp, Artificial
  Synthesized sequence
ccctttaatcagatgcgtcggagatgagtagacgagtggggcagtgggccggaagcggcagctccggcgcaggctccggggatatccagctgacacagagcccttcatcactctccgcctctgttggagatagagtcacaacactgctaatgcgagtcagtgaccatggtcgcccttattactacca

SEQ ID NO: 689 - DNA, 183 bp, Artificial
  Synthesized sequence
ccctttaatcagatgcgtcggagatgagtagacgagtggggcagtggcctctgttggagatagagtcacaatgacttgtagggcctcctcttccgtgtcatacatccactggttccagcagaagcccggtaaggctccactgctaatgcgagtcagtgaccatggtcgcccttattactacca

SEQ ID NO: 690 - DNA, 188 bp, Artificial
  Synthesized sequence
ccctttaatcagatgcgtcggagatgagtagacgagtggggcagtggcagaagcccggtaaggctcccaagccttggatttatgccacatccaatctggcctcaggtgtgcccgtccgcttctccggtagcggatctgggaccactgctaatgcgagtcagtgaccatggtcgcccttattactacca

SEQ ID NO: 691 - DNA, 186 bp, Artificial
  Synthesized sequence
ccctttaatcagatgcgtcggagatgagtagacgagtggggcagtgtccggtagcggatctgggactgattatactttcacaattagctctctgcagccagaagatattgcaacttactattgccaacagtggacatccacactgctaatgcgagtcagtgaccatggtcgcccttattactacca

SEQ ID NO: 692 - DNA, 184 bp, Artificial
  Synthesized sequence
ccctttaatcagatgcgtcggagatgagtagacgagtggggcagtgctattgccaacagtggacatccaatcctcctacttttggaggggggactaagctcgaaataaaggggcccagtcaaaactgtaaccgcacttcactgctaatgcgagtcagtgaccatggtcgcccttattactacca

SEQ ID NO: 693 - DNA, 187 bp, Artificial
  Synthesized sequence
ccctttaatcagatgcgtcgctttgggctttcagatgagcgcagtgtttttggcagatcattaacggcggcccagccggccaggcgccaggttcagctccaagagtcaggtcctgggctggttaagccttctgagacactgcactgcccgaccgacagaaatctttgggtcgcccttattactacca

SEQ ID NO: 694 - DNA, 183 bp, Artificial
  Synthesized sequence
ccctttaatcagatgcgtcgctttgggctttcagatgagcgcagtgctggttaagccttctgagacactgagcctgacctgcaccgttagcggcttctccctgatcggctacgatctgaactggattcggcagccaccactgcccgaccgacagaaatctttgggtcgcccttattactacca

SEQ ID NO: 695 - DNA, 190 bp, Artificial
  Synthesized sequence
ccctttaatcagatgcgtcgctttgggctttcagatgagcgcagtggaactggattcggcagccacccggaaagggcctggaatggattggcataatctggggagacgggacaactgactataattctgccgttaagtcacgcgcactgcccgaccgacagaaatctttgggtcgcccttattactacca

SEQ ID NO: 696 - DNA, 185 bp, Artificial
  Synthesized sequence
ccctttaatcagatgcgtcgctttgggctttcagatgagcgcagtgactataattctgccgttaagtcacgcgtgaccatatctaaagacacaagcaagaaccagttcagcctgaaactgtcctcagtcacagcagcagcactgcccgaccgacagaaatctttgggtcgcccttattactacca

SEQ ID NO: 697 - DNA, 188 bp, Artificial
  Synthesized sequence
ccctttaatcagatgcgtcgctttgggctttcagatgagcgcagtgctgtcctcagtcacagcagcagatactgctgtgtattactgtgcccgcgggggctattggtacgctacctcatattactttgattactgggggcagcactgcccgaccgacagaaatctttgggtcgcccttattactacca

SEQ ID NO: 698 - DNA, 181 bp, Artificial
  Synthesized sequence
ccctttaatcagatgcgtcgctttgggctttcagatgagcgcagtgatattactttgattactgggggcagggcaccctggtgaccgtctcctctggaggctctggtgggtctggaggagcatctggggccgggacactgcccgaccgacagaaatctttgggtcgcccttattactacca

SEQ ID NO: 699 - DNA, 187 bp, Artificial
  Synthesized sequence
ccctttaatcagatgcgtcgctttgggctttcagatgagcgcagtggagcatctggggccgggagcggcgggggggatattcagatgactcaatcaccctcaagcctctcagcctcagtcggggaccgggtgacaatcacccactgcccgaccgacagaaatctttgggtcgcccttattactacca

SEQ ID NO: 700 - DNA, 187 bp, Artificial
  Synthesized sequence
ccctttaatcagatgcgtcgctttgggctttcagatgagcgcagtgggggaccgggtgacaatcacctgtagggcttcacaaagcatatccaacaatctgaattggtaccagcaaaaaccaggaaaagccccaaaactcctcactgcccgaccgacagaaatctttgggtcgcccttattactacca

SEQ ID NO: 701 - DNA, 187 bp, Artificial
  Synthesized sequence
ccctttaatcagatgcgtcgctttgggctttcagatgagcgcagtgaccaggaaaagccccaaaactcctgatatactatacctcccggttccacagcggggtgcctagcaggttcagcggctccggcagcggcactgattcactgcccgaccgacagaaatctttgggtcgcccttattactacca

SEQ ID NO: 702 - DNA, 187 bp, Artificial
  Synthesized sequence
ccctttaatcagatgcgtcgctttgggctttcagatgagcgcagtgccggcagcggcactgatttcactttcaccatttcctccctgcaaccagaggacattgcaacttattattgccagcaggagcataccctgccatatcactgcccgaccgacagaaatctttgggtcgcccttattactacca

SEQ ID NO: 703 - DNA, 175 bp, Artificial
  Synthesized sequence
ccctttaatcagatgcgtcgctttgggctttcagatgagcgcagtggcaggagcataccctgccatatactttcggccagggtacaaagctggagataaaggggcccctgtcaccctatgtagtcccttcactgcccgaccgacagaaatctttgggtcgcccttattactacca

SEQ ID NO: 704 - DNA, 182 bp, Artificial
  Synthesized sequence
ccctttaatcagatgcgtcgtgtcatatgctaacgtccgtgcagtgtttatgatctccgtacacgagcggcccagccggccaggcgcgaagtgcaactggtcgaaagcgggggtggactggtgcagcctgggggcacactgcttccgctaagaaagtagccaggtcgcccttattactacca

SEQ ID NO: 705 - DNA, 184 bp, Artificial
  Synthesized sequence
ccctttaatcagatgcgtcgtgtcatatgctaacgtccgtgcagtgtggtgcagcctgggggcagcctgcgcctgagctgtgcagcttcaggctttaccttcatcagctacgctatgtcttgggtgagacaggccccccactgcttccgctaagaaagtagccaggtcgcccttattactacca

SEQ ID NO: 706 - DNA, 188 bp, Artificial
  Synthesized sequence
ccctttaatcagatgcgtcgtgtcatatgctaacgtccgtgcagtgcttgggtgagacaggcccccggaaaaggactcgaatgggtggctagcatctcaagcggtggcaatacatactaccccgacagcgtcaagggccggtcactgcttccgctaagaaagtagccaggtcgcccttattactacca

SEQ ID NO: 707 - DNA, 188 bp, Artificial
  Synthesized sequence
ccctttaatcagatgcgtcgtgtcatatgctaacgtccgtgcagtgacagcgtcaagggccggtttaccatctcacgcgacaatgccaagaattccctgtacctgcagatgaactccctgcgcgctgaagatacagccgtctcactgcttccgctaagaaagtagccaggtcgcccttattactacca

SEQ ID NO: 708 - DNA, 188 bp, Artificial
  Synthesized sequence
ccctttaatcagatgcgtcgtgtcatatgctaacgtccgtgcagtgcgcgctgaagatacagccgtctattattgcgctcggctggacggctactactttggcttcgcatactggggccaggggaccctggtgacagtcagccactgcttccgctaagaaagtagccaggtcgcccttattactacca

SEQ ID NO: 709 - DNA, 188 bp, Artificial
  Synthesized sequence
ccctttaatcagatgcgtcgtgtcatatgctaacgtccgtgcagtggggaccctggtgacagtcagctccggggggagcgccggctcagggtcctccggtggtgcctctggctcagggggggacattcaaatgacacagagccactgcttccgctaagaaagtagccaggtcgcccttattactacca

SEQ ID NO: 710 - DNA, 188 bp, Artificial
  Synthesized sequence
ccctttaatcagatgcgtcgtgtcatatgctaacgtccgtgcagtggggggacattcaaatgacacagagcccctcttctctctcagctagcgtgggcgaccgcgttacaattacttgcaaagccagcgaatccgtcgataacactgcttccgctaagaaagtagccaggtcgcccttattactacca

SEQ ID NO: 711 - DNA, 186 bp, Artificial
  Synthesized sequence
ccctttaatcagatgcgtcgtgtcatatgctaacgtccgtgcagtgagccagcgaatccgtcgataactatgggaagtccctgatgcactggtatcaacagaaacctggaaaggctcccaaactgctcatctaccgggctcactgcttccgctaagaaagtagccaggtcgcccttattactacca

SEQ ID NO: 712 - DNA, 188 bp, Artificial
  Synthesized sequence
ccctttaatcagatgcgtcgtgtcatatgctaacgtccgtgcagtgcaaactgctcatctaccgggcttcaaacctggagagcggtgtgccctcacggttctccggatctggaagcgggactgactttaccctcaccatctccactgcttccgctaagaaagtagccaggtcgcccttattactacca

SEQ ID NO: 713 - DNA, 187 bp, Artificial
  Synthesized sequence
ccctttaatcagatgcgtcgtgtcatatgctaacgtccgtgcagtggactgactttaccctcaccatctcctcactccaaccagaggatttcgctacatattattgccagcaatctaacgaggatccatggacattcggggcactgcttccgctaagaaagtagccaggtcgcccttattactacca

SEQ ID NO: 714 - DNA, 166 bp, Artificial
  Synthesized sequence
ccctttaatcagatgcgtcgtgtcatatgctaacgtccgtgcagtgcgaggatccatggacattcggggggggcacaaaggttgaaatcaaggggcccacttctttggaacgacaacgttcactgcttccgctaagaaagtagccaggtcgcccttattactacca

SEQ ID NO: 715 - DNA, 185 bp, Artificial
  Synthesized sequence
ccctttaatcagatgcgtcgttgcgacatcacaattctcggcagtgttagtgccatgttatccctgaaggcccagccggccaggcgcgaggtgcaactcgtccagagcggcgccgaggttaagaagcctggcgagtccccactgcacgcatgaagtctcgaagtaggtcgcccttattactacca

SEQ ID NO: 716 - DNA, 183 bp, Artificial
  Synthesized sequence
ccctttaatcagatgcgtcgttgcgacatcacaattctcggcagtggttaagaagcctggcgagtccctgaaaatttcctgcaaaggcagcgggtactctttcactacatactggctgggttgggtgcggcagatgccactgcacgcatgaagtctcgaagtaggtcgcccttattactacca

SEQ ID NO: 717 - DNA, 187 bp, Artificial
  Synthesized sequence
ccctttaatcagatgcgtcgttgcgacatcacaattctcggcagtggggttgggtgcggcagatgcccgggaaggggctggattggatcggcataatgtccccagtggattcagacatacgctatagcccctccttccaggcactgcacgcatgaagtctcgaagtaggtcgcccttattactacca

SEQ ID NO: 718 - DNA, 187 bp, Artificial
  Synthesized sequence
ccctttaatcagatgcgtcgttgcgacatcacaattctcggcagtgacgctatagcccctccttccagggtcaggtgaccatgagcgtcgataagagcattactaccgcctacctccagtggaattccctgaaggcctctgcactgcacgcatgaagtctcgaagtaggtcgcccttattactacca

SEQ ID NO: 719 - DNA, 183 bp, Artificial
  Synthesized sequence
ccctttaatcagatgcgtcgttgcgacatcacaattctcggcagtggtggaattccctgaaggcctctgatacagccatgtactactgcgcccgcagacgcccaggacagggatacttcgacttctggggccagggacactgcacgcatgaagtctcgaagtaggtcgcccttattactacca

SEQ ID NO: 720 - DNA, 182 bp, Artificial
  Synthesized sequence
ccctttaatcagatgcgtcgttgcgacatcacaattctcggcagtgtcgacttctggggccagggaaccctcgtgaccgtttcaagcggcggggcagggtctggcgcaggaagcggcagcagcggagccggatctgcactgcacgcatgaagtctcgaagtaggtcgcccttattactacca

SEQ ID NO: 721 - DNA, 187 bp, Artificial
  Synthesized sequence
ccctttaatcagatgcgtcgttgcgacatcacaattctcggcagtgagcagcggagccggatctggggatattcagatgacccagtctccttcttccctctctgctagcgtcggcgatagggttacaatcacttgcagggccactgcacgcatgaagtctcgaagtaggtcgcccttattactacca

SEQ ID NO: 722 - DNA, 187 bp, Artificial
  Synthesized sequence
ccctttaatcagatgcgtcgttgcgacatcacaattctcggcagtgtagggttacaatcacttgcagggccagccagggcatatcatcttggctggcttggtatcagcagaagccagaaaaggcccctaagagcctcatatcactgcacgcatgaagtctcgaagtaggtcgcccttattactacca

SEQ ID NO: 723 - DNA, 187 bp, Artificial
  Synthesized sequence
ccctttaatcagatgcgtcgttgcgacatcacaattctcggcagtgaaggcccctaagagcctcatatatgctgccagctccctgcagtccggcgtgccctcccgcttctcaggctcaggttcagggacagacttcacactcactgcacgcatgaagtctcgaagtaggtcgcccttattactacca

SEQ ID NO: 724 - DNA, 191 bp, Artificial
  Synthesized sequence
ccctttaatcagatgcgtcgttgcgacatcacaattctcggcagtgaggttcagggacagacttcacactgacaatctcctccctccagccagaggatttcgccacctattattgccaacagtacaatatctacccttacaccttcactgcacgcatgaagtctcgaagtaggtcgcccttattactacca

SEQ ID NO: 725 - DNA, 176 bp, Artificial
  Synthesized sequence
ccctttaatcagatgcgtcgttgcgacatcacaattctcggcagtgaacagtacaatatctacccttacacctttggccagggcaccaaactggaaatcaaggggcccgggtccgtatatgtgtgactttcactgcacgcatgaagtctcgaagtaggtcgcccttattactacca

SEQ ID NO: 726 - DNA, 182 bp, Artificial
  Synthesized sequence
ccctttaatcagatgcgtcgtcagtatggcgtcttgaagtgcagtgttttatacatctggacgcctccggcccagccggccaggcgcgaagtgcaactggtggagtctgggggaggcctggttcagcccggtgggacactgccataatagaggtcgggccatggtcgcccttattactacca

SEQ ID NO: 727 - DNA, 183 bp, Artificial
  Synthesized sequence
ccctttaatcagatgcgtcgtcagtatggcgtcttgaagtgcagtgctggttcagcccggtgggagcctgcggctgtcctgcgccgcttccggctactcattcaccggatactacatccattgggtgaggcaggccccactgccataatagaggtcgggccatggtcgcccttattactacca

SEQ ID NO: 728 - DNA, 190 bp, Artificial
  Synthesized sequence
ccctttaatcagatgcgtcgtcagtatggcgtcttgaagtgcagtgccattgggtgaggcaggcccctgggaagggcctggaatgggtggctagagtcattcctaatgccggtggaacaagctacaatcagaaattcaaggggccactgccataatagaggtcgggccatggtcgcccttattactacca

SEQ ID NO: 729 - DNA, 185 bp, Artificial
  Synthesized sequence
ccctttaatcagatgcgtcgtcagtatggcgtcttgaagtgcagtgcaagctacaatcagaaattcaaggggcggtttaccctgagcgttgacaactctaagaatactgcatatctgcagatgaactctctgcgggccgcactgccataatagaggtcgggccatggtcgcccttattactacca

SEQ ID NO: 730 - DNA, 186 bp, Artificial
  Synthesized sequence
ccctttaatcagatgcgtcgtcagtatggcgtcttgaagtgcagtgcagatgaactctctgcgggccgaggacaccgccgtgtattactgcgccagggaaggaatctattggtggggccaaggtaccctggtgacagtctcactgccataatagaggtcgggccatggtcgcccttattactacca

SEQ ID NO: 731 - DNA, 187 bp, Artificial
  Synthesized sequence
ccctttaatcagatgcgtcgtcagtatggcgtcttgaagtgcagtgccaaggtaccctggtgacagtctcttccgggggctcaggaggatctggaggtgcatccggcgccggaagcggagggggcgacatccagatgacaccactgccataatagaggtcgggccatggtcgcccttattactacca

SEQ ID NO: 732 - DNA, 187 bp, Artificial
  Synthesized sequence
ccctttaatcagatgcgtcgtcagtatggcgtcttgaagtgcagtggggggcgacatccagatgacacagtccccttcttctctctctgcatccgttggagatagagttacaattacttgtcggagctctcagtcactggtcactgccataatagaggtcgggccatggtcgcccttattactacca

SEQ ID NO: 733 - DNA, 187 bp, Artificial
  Synthesized sequence
ccctttaatcagatgcgtcgtcagtatggcgtcttgaagtgcagtggtcggagctctcagtcactggtgcacagcaacggtaacacattcctgcactggtaccagcagaaacctggcaaagcccctaagctgctgatataccactgccataatagaggtcgggccatggtcgcccttattactacca

SEQ ID NO: 734 - DNA, 186 bp, Artificial
  Synthesized sequence
ccctttaatcagatgcgtcgtcagtatggcgtcttgaagtgcagtgaaagcccctaagctgctgatatacacagtctccaaccggttctctggagtgccctccaggttttcaggaagcgggtcagggacagactttaccccactgccataatagaggtcgggccatggtcgcccttattactacca

SEQ ID NO: 735 - DNA, 186 bp, Artificial
  Synthesized sequence
ccctttaatcagatgcgtcgtcagtatggcgtcttgaagtgcagtgcgggtcagggacagactttaccctgactatctcctctctgcaacctgaggatttcgccacctatttctgcagccaaactacccatgttccctggcactgccataatagaggtcgggccatggtcgcccttattactacca

SEQ ID NO: 736 - DNA, 176 bp, Artificial
  Synthesized sequence
ccctttaatcagatgcgtcgtcagtatggcgtcttgaagtgcagtggccaaactacccatgttccctggacttttggtcaggggaccaaggttgagatcaaggggccccgccataataggggttctctttcactgccataatagaggtcgggccatggtcgcccttattactacca

SEQ ID NO: 737 - DNA, 193 bp, Artificial
  Synthesized sequence
ccctttaatcagatgcgtcgtcatgtcgtgaccagtagacgcagtgtttcctcgattctccaatcaggggcccagccggccaggcgcgaagtccaactcgtggagtccgggggaggcctggtgcagcccggtgggagcctgaggctccactgcgacgaagttcactagacccaggtcgcccttattactacca

SEQ ID NO: 738 - DNA, 193 bp, Artificial
  Synthesized sequence
ccctttaatcagatgcgtcgtcatgtcgtgaccagtagacgcagtgcggtgggagcctgaggctctcctgtgccgccagcggcttcacattctcttcctacggtatgtcatgggtcaggcaggcccccggaaaaggcctggaatgggcactgcgacgaagttcactagacccaggtcgcccttattactacca

SEQ ID NO: 739 - DNA, 192 bp, Artificial
  Synthesized sequence
ccctttaatcagatgcgtcgtcatgtcgtgaccagtagacgcagtgcccggaaaaggcctggaatgggtcgcaaccataacatccggcggcagctatacatactacgtggatagcgttaaggggaggttcacaatttcccgggacacactgcgacgaagttcactagacccaggtcgcccttattactacca

SEQ ID NO: 740 - DNA, 193 bp, Artificial
  Synthesized sequence
ccctttaatcagatgcgtcgtcatgtcgtgaccagtagacgcagtggaggttcacaatttcccgggacaacgccaaaaacacactgtacctgcagatgaactctctgcgggccgaggataccgctgtgtactattgcgtgaggatagcactgcgacgaagttcactagacccaggtcgcccttattactacca

SEQ ID NO: 741 - DNA, 193 bp, Artificial
  Synthesized sequence
ccctttaatcagatgcgtcgtcatgtcgtgaccagtagacgcagtgctgtgtactattgcgtgaggataggcgaagatgctctggactactggggacaggggactctggtcacagtgtcaagcggcggcagcgccggctcaggtagccactgcgacgaagttcactagacccaggtcgcccttattactacca

SEQ ID NO: 742 - DNA, 193 bp, Artificial
  Synthesized sequence
ccctttaatcagatgcgtcgtcatgtcgtgaccagtagacgcagtgagcgccggctcaggtagctctgggggtgcctctggatccggcggcgatatccagatgacacaatctccttccagcctgtccgcctccgtgggtgacagggtcactgcgacgaagttcactagacccaggtcgcccttattactacca

SEQ ID NO: 743 - DNA, 193 bp, Artificial
  Synthesized sequence
ccctttaatcagatgcgtcgtcatgtcgtgaccagtagacgcagtggcctccgtgggtgacagggtgaccattacatgtagagcatcacaggacatcgcagggtccctgaattggctgcaacaaaagcctgggaaagctatcaaaagcactgcgacgaagttcactagacccaggtcgcccttattactacca

SEQ ID NO: 744 - DNA, 193 bp, Artificial
  Synthesized sequence
ccctttaatcagatgcgtcgtcatgtcgtgaccagtagacgcagtgaaagcctgggaaagctatcaaaaggctgatttacgcaacaagctctctcgacagcggcgttcctaagagattctctggctctaggtcaggaagcgattatacactgcgacgaagttcactagacccaggtcgcccttattactacca

SEQ ID NO: 745 - DNA, 191 bp, Artificial
  Synthesized sequence
ccctttaatcagatgcgtcgtcatgtcgtgaccagtagacgcagtggctctaggtcaggaagcgattataccctgactatctctagcctccagcctgaagattttgccacttattattgcctccagtacgggtctttcccacctacactgcgacgaagttcactagacccaggtcgcccttattactacca

SEQ ID NO: 746 - DNA, 174 bp, Artificial
  Synthesized sequence
ccctttaatcagatgcgtcgtcatgtcgtgaccagtagacgcagtgcagtacgggtctttcccacctacctttggtcagggcacaaaagtcgagataaaagggccccgcatgttttagcctaacgattcactgcgacgaagttcactagacccaggtcgcccttattactacca

SEQ ID NO: 747 - DNA, 187 bp, Artificial
  Synthesized sequence
ccctttaatcagatgcgtcgaactaacggatttaagcgcggcagtgttgcttaacgcatttcaagcacggcccagccggccaggcgcgaagttcagctggtggagtccggggggggtctggtccagccaggaggttcactccactgccggacgaagcaacatatgttggtcgcccttattactacca

SEQ ID NO: 748 - DNA, 180 bp, Artificial
  Synthesized sequence
ccctttaatcagatgcgtcgaactaacggatttaagcgcggcagtggtccagccaggaggttcactccgcctctcttgcgcagcctcaggcttcacctttagctcttacgtgatgtcctgggtcaggcaggccccactgccggacgaagcaacatatgttggtcgcccttattactacca

SEQ ID NO: 749 - DNA, 188 bp, Artificial
  Synthesized sequence
ccctttaatcagatgcgtcgaactaacggatttaagcgcggcagtgcctgggtcaggcaggcccctggcaagggtctcgaatgggttgccacaatctcttcaggcggaagctacacctactatcccgactctgttaaaggaacactgccggacgaagcaacatatgttggtcgcccttattactacca

SEQ ID NO: 750 - DNA, 187 bp, Artificial
  Synthesized sequence
ccctttaatcagatgcgtcgaactaacggatttaagcgcggcagtgtactatcccgactctgttaaaggaagattcacaatttccagagataacgccaaaaacacactgtacctgcaaatgaattcactgagagctgaggacactgccggacgaagcaacatatgttggtcgcccttattactacca

SEQ ID NO: 751 - DNA, 186 bp, Artificial
  Synthesized sequence
ccctttaatcagatgcgtcgaactaacggatttaagcgcggcagtgaatgaattcactgagagctgaggatactgctgtgtactactgcgccagacgcggtgactccatgatcaccaccgactattggggtcaggggactcactgccggacgaagcaacatatgttggtcgcccttattactacca

SEQ ID NO: 752 - DNA, 185 bp, Artificial
  Synthesized sequence
ccctttaatcagatgcgtcgaactaacggatttaagcgcggcagtgccgactattggggtcaggggactctggtcaccgtgtcatccgggggagccgggagcggggctggcagcggatcttctggagcaggttctggcgcactgccggacgaagcaacatatgttggtcgcccttattactacca

SEQ ID NO: 753 - DNA, 187 bp, Artificial
  Synthesized sequence
ccctttaatcagatgcgtcgaactaacggatttaagcgcggcagtgtcttctggagcaggttctggcgacatccagatgacacaaagcccttcatccctctctgcatctgtcggcgatcgcgtgactataacctgcaaagccactgccggacgaagcaacatatgttggtcgcccttattactacca

SEQ ID NO: 754 - DNA, 187 bp, Artificial
  Synthesized sequence
ccctttaatcagatgcgtcgaactaacggatttaagcgcggcagtgtcgcgtgactataacctgcaaagcctcccaggacgttggaactgccgttgcttggtaccagcagaaacccggcaaggcacctaagctgctgatctcactgccggacgaagcaacatatgttggtcgcccttattactacca

SEQ ID NO: 755 - DNA, 186 bp, Artificial
  Synthesized sequence
ccctttaatcagatgcgtcgaactaacggatttaagcgcggcagtgaaggcacctaagctgctgatctactgggctagcacaaggcatactggggtgcccagccgcttctccggttccggcagcggtacagatttcacaccactgccggacgaagcaacatatgttggtcgcccttattactacca

SEQ ID NO: 756 - DNA, 187 bp, Artificial
  Synthesized sequence
ccctttaatcagatgcgtcgaactaacggatttaagcgcggcagtgcggcagcggtacagatttcacactcactattagctctctgcagcctgaagacttcgccacctactattgccagcagtactctagctaccggacctcactgccggacgaagcaacatatgttggtcgcccttattactacca

SEQ ID NO: 757 - DNA, 173 bp, Artificial
  Synthesized sequence
ccctttaatcagatgcgtcgaactaacggatttaagcgcggcagtgagcagtactctagctaccggaccttcggacagggaacaaaagtggagatcaaggggcccgtaggctgaacgacctatcattcactgccggacgaagcaacatatgttggtcgcccttattactacca

SEQ ID NO: 758 - DNA, 187 bp, Artificial
  Synthesized sequence
ccctttaatcagatgcgtcgcattttctgttccccagtgggcagtgttcttttatgttcctcgcagggggcccagccggccaggcgccaggtgcagctgcagcagtccggcgccgagctggtgaagccaggtgcatctgttcactgcggggtgacaatctaactcgaggtcgcccttattactacca

SEQ ID NO: 759 - DNA, 182 bp, Artificial
  Synthesized sequence
ccctttaatcagatgcgtcgcattttctgttccccagtgggcagtgggtgaagccaggtgcatctgttaagctgtcctgcaaggcatccggctatactttcacctcctacgatatcaactgggttcggcagaggcccactgcggggtgacaatctaactcgaggtcgcccttattactacca

SEQ ID NO: 760 - DNA, 189 bp, Artificial
  Synthesized sequence
ccctttaatcagatgcgtcgcattttctgttccccagtgggcagtgactgggttcggcagaggcctgagcaaggactggagtggattgggtggatcttccccggagatggatctaccaagtataacgagaagttcaaggggaacactgcggggtgacaatctaactcgaggtcgcccttattactacca

SEQ ID NO: 761 - DNA, 187 bp, Artificial
  Synthesized sequence
ccctttaatcagatgcgtcgcattttctgttccccagtgggcagtgaagtataacgagaagttcaaggggaaagccaccctgaccacagataaaagctcaagcaccgcctatatgcagctctctcggctgacatctgaagacactgcggggtgacaatctaactcgaggtcgcccttattactacca

SEQ ID NO: 762 - DNA, 188 bp, Artificial
  Synthesized sequence
ccctttaatcagatgcgtcgcattttctgttccccagtgggcagtggctctctcggctgacatctgaagattctgccgtctatttttgcgctcgggaggactactacgacaactcatattattttgactactggggtcagggcactgcggggtgacaatctaactcgaggtcgcccttattactacca

SEQ ID NO: 763 - DNA, 183 bp, Artificial
  Synthesized sequence
ccctttaatcagatgcgtcgcattttctgttccccagtgggcagtgattattttgactactggggtcaggggacaacactcactgtctccagcggcggctcaggtgggagcggcggggcttctggtgccggatccggcactgcggggtgacaatctaactcgaggtcgcccttattactacca

SEQ ID NO: 764 - DNA, 187 bp, Artificial
  Synthesized sequence
ccctttaatcagatgcgtcgcattttctgttccccagtgggcagtggcttctggtgccggatccggaggcggtgatatccagatgacccagacaacttcaagcctgtccgcctcactgggggatcgggtcaccatttcttgcactgcggggtgacaatctaactcgaggtcgcccttattactacca

SEQ ID NO: 765 - DNA, 187 bp, Artificial
  Synthesized sequence
ccctttaatcagatgcgtcgcattttctgttccccagtgggcagtgggggatcgggtcaccatttcttgcagagcctctcaggatatcagcaattacctgaattggtaccagcaaaaacccgatggaacagtgaaactgctcactgcggggtgacaatctaactcgaggtcgcccttattactacca

SEQ ID NO: 766 - DNA, 187 bp, Artificial
  Synthesized sequence
ccctttaatcagatgcgtcgcattttctgttccccagtgggcagtgacccgatggaacagtgaaactgctgatctactacacatctcggctgcatagcggagtgccctccaggttcagcggctccgggtctggcacagactcactgcggggtgacaatctaactcgaggtcgcccttattactacca

SEQ ID NO: 767 - DNA, 187 bp, Artificial
  Synthesized sequence
ccctttaatcagatgcgtcgcattttctgttccccagtgggcagtgtccgggtctggcacagactacagcctgaccatcagcaacctggaacaggaggacattgccacctatttttgtcaacaaggaaataccctcccttgcactgcggggtgacaatctaactcgaggtcgcccttattactacca

SEQ ID NO: 768 - DNA, 178 bp, Artificial
  Synthesized sequence
ccctttaatcagatgcgtcgcattttctgttccccagtgggcagtgtcaacaaggaaataccctcccttggacatttgggggaggcaccaagctggaaattaaggggcccagtgcttatgaaagtcccgattcactgcggggtgacaatctaactcgaggtcgcccttattactacca

SEQ ID NO: 769 - DNA, 188 bp, Artificial
  Synthesized sequence
ccctttaatcagatgcgtcgatttgcctaaccactccactgcagtgttgtgggcgttagcaaattacaggcccagccggccaggcgccaggtgcaactccaggaatccggtcccggcctggtgaagccatctcagacactgtcactgcactgtaccgaaaagctctgaggtcgcccttattactacca

SEQ ID NO: 770 - DNA, 188 bp, Artificial
  Synthesized sequence
ccctttaatcagatgcgtcgatttgcctaaccactccactgcagtgtggtgaagccatctcagacactgtccctgacctgcacagtttccggcggcagcatctctagcggagactatttctggtcctggatcagacagctcccactgcactgtaccgaaaagctctgaggtcgcccttattactacca

SEQ ID NO: 771 - DNA, 192 bp, Artificial
  Synthesized sequence
ccctttaatcagatgcgtcgatttgcctaaccactccactgcagtgtggtcctggatcagacagctcccaggcaagggcctggagtggatagggcatattcataactctggaacaacctactataatccctctctcaaatcacgggcactgcactgtaccgaaaagctctgaggtcgcccttattactacca

SEQ ID NO: 772 - DNA, 186 bp, Artificial
  Synthesized sequence
ccctttaatcagatgcgtcgatttgcctaaccactccactgcagtgtactataatccctctctcaaatcacgggttactatctccgtggacacttccaagaaacagttctccctcagactgtcctcagttaccgcagccgcactgcactgtaccgaaaagctctgaggtcgcccttattactacca

SEQ ID NO: 773 - DNA, 188 bp, Artificial
  Synthesized sequence
ccctttaatcagatgcgtcgatttgcctaaccactccactgcagtgctgtcctcagttaccgcagccgacaccgctgtgtattactgcgcaagggacagggggggcgactattactacggcatggacgtgtggggccaaggtcactgcactgtaccgaaaagctctgaggtcgcccttattactacca

SEQ ID NO: 774 - DNA, 188 bp, Artificial
  Synthesized sequence
ccctttaatcagatgcgtcgatttgcctaaccactccactgcagtgtggacgtgtggggccaaggtacaactgttaccgtttcctcaggtggatcagccggcagcggatcttctggtggcgcctccggatctggcggagaaacactgcactgtaccgaaaagctctgaggtcgcccttattactacca

SEQ ID NO: 775 - DNA, 186 bp, Artificial
  Synthesized sequence
ccctttaatcagatgcgtcgatttgcctaaccactccactgcagtgctccggatctggcggagaaattgtgctcactcaatccccagggacactgtccctcagccctggcgaacgggccactctgtcctgcagggctagccactgcactgtaccgaaaagctctgaggtcgcccttattactacca

SEQ ID NO: 776 - DNA, 188 bp, Artificial
  Synthesized sequence
ccctttaatcagatgcgtcgatttgcctaaccactccactgcagtgcactctgtcctgcagggctagccagggcattagccggagctacctggcctggtatcagcaaaagcctgggcaggccccctctctgctgatctatggcactgcactgtaccgaaaagctctgaggtcgcccttattactacca

SEQ ID NO: 777 - DNA, 188 bp, Artificial
  Synthesized sequence
ccctttaatcagatgcgtcgatttgcctaaccactccactgcagtgggccccctctctgctgatctatggtgcatcctcccgcgccaccgggatccctgacagattttccggatccggtagcggtacagacttcactctgaccactgcactgtaccgaaaagctctgaggtcgcccttattactacca

SEQ ID NO: 778 - DNA, 188 bp, Artificial
  Synthesized sequence
ccctttaatcagatgcgtcgatttgcctaaccactccactgcagtgtagcggtacagacttcactctgacaatttcccgcctggagcccgaggattttgctgtgtattactgccagcaatttggttcttcaccatggaccttcactgcactgtaccgaaaagctctgaggtcgcccttattactacca

SEQ ID NO: 779 - DNA, 172 bp, Artificial
  Synthesized sequence
ccctttaatcagatgcgtcgatttgcctaaccactccactgcagtgatttggttcttcaccatggacctttggtcaagggacaaaggtggaaataaaggggcccccgaactggacgcataaaatttcactgcactgtaccgaaaagctctgaggtcgcccttattactacca

SEQ ID NO: 780 - DNA, 189 bp, Artificial
  Synthesized sequence
ccctttaatcagatgcgtcgtgacttatgaacctttgcgcgcagtgttagagattattaggcgtggggggcccagccggccaggcgccaggtccagctggttcaaagcggagccgaggttaaaaaacctggttctagcgtgaacactgcattaacgactactcctgggcggtcgcccttattactacca

SEQ ID NO: 781 - DNA, 189 bp, Artificial
  Synthesized sequence
ccctttaatcagatgcgtcgtgacttatgaacctttgcgcgcagtgtaaaaaacctggttctagcgtgaaagtgagctgcaaggcctctggctacgcattctcttacagctggatcaattgggtgcgccaggccccaggtcagcactgcattaacgactactcctgggcggtcgcccttattactacca

SEQ ID NO: 782 - DNA, 189 bp, Artificial
  Synthesized sequence
ccctttaatcagatgcgtcgtgacttatgaacctttgcgcgcagtgcgccaggccccaggtcagggtctggagtggatgggcaggatctttccaggagacggagataccgattacaacggcaagtttaaagggagggtgactacactgcattaacgactactcctgggcggtcgcccttattactacca

SEQ ID NO: 783 - DNA, 188 bp, Artificial
  Synthesized sequence
ccctttaatcagatgcgtcgtgacttatgaacctttgcgcgcagtggcaagtttaaagggagggtgactataaccgctgacaagagcacttcaacagcctatatggaactcagctctctcagaagcgaggatacagcagtctcactgcattaacgactactcctgggcggtcgcccttattactacca

SEQ ID NO: 784 - DNA, 189 bp, Artificial
  Synthesized sequence
ccctttaatcagatgcgtcgtgacttatgaacctttgcgcgcagtgcagaagcgaggatacagcagtctactattgtgctcggaatgtctttgacgggtactggctggtgtactggggccagggaaccctggtcacagttagccactgcattaacgactactcctgggcggtcgcccttattactacca

SEQ ID NO: 785 - DNA, 189 bp, Artificial
  Synthesized sequence
ccctttaatcagatgcgtcgtgacttatgaacctttgcgcgcagtgagggaaccctggtcacagttagcagcgcaggtggggccggctctggggcagggagcggctcctctggcgccggcagcggggacatagtgatgacacacactgcattaacgactactcctgggcggtcgcccttattactacca

SEQ ID NO: 786 - DNA, 189 bp, Artificial
  Synthesized sequence
ccctttaatcagatgcgtcgtgacttatgaacctttgcgcgcagtgagcggggacatagtgatgacacaaactcctctgtctctgccagttacccccggagaacccgccagcatttcttgtagatcctctaaaagcctgctgccactgcattaacgactactcctgggcggtcgcccttattactacca

SEQ ID NO: 787 - DNA, 187 bp, Artificial
  Synthesized sequence
ccctttaatcagatgcgtcgtgacttatgaacctttgcgcgcagtgtgtagatcctctaaaagcctgctgcatagcaatgggatcacctacctgtactggtatctgcagaaacccggccaatcccctcagctgctgatttacactgcattaacgactactcctgggcggtcgcccttattactacca

SEQ ID NO: 788 - DNA, 189 bp, Artificial
  Synthesized sequence
ccctttaatcagatgcgtcgtgacttatgaacctttgcgcgcagtgaatcccctcagctgctgatttaccaaatgtccaacctggtgtcaggagtcccagatcggttcagcggatccggaagcggtactgattttaccctcaacactgcattaacgactactcctgggcggtcgcccttattactacca

SEQ ID NO: 789 - DNA, 189 bp, Artificial
  Synthesized sequence
ccctttaatcagatgcgtcgtgacttatgaacctttgcgcgcagtggaagcggtactgattttaccctcaaaatatcaagggtggaagccgaggacgtgggcgtgtactattgcgcccagaatctggaactcccttatacattcactgcattaacgactactcctgggcggtcgcccttattactacca

SEQ ID NO: 790 - DNA, 174 bp, Artificial
  Synthesized sequence
ccctttaatcagatgcgtcgtgacttatgaacctttgcgcgcagtgcagaatctggaactcccttatacattcggaggcggcacaaaagtggaaataaaagggccctgaagggaaataccagccttttcactgcattaacgactactcctgggcggtcgcccttattactacca

SEQ ID NO: 791 - DNA, 188 bp, Artificial
  Synthesized sequence
ccctttaatcagatgcgtcgataggattagctgatgggccgcagtgtttaggattactgctcggtgacggcccagccggccaggcgcgaggtgcagctggtgcaaagcgggccaggcctcgtccagcctgggggatctgttacactgcgaccttagtcggaacacagaggtcgcccttattactacca

SEQ ID NO: 792 - DNA, 184 bp, Artificial
  Synthesized sequence
ccctttaatcagatgcgtcgataggattagctgatgggccgcagtgtccagcctgggggatctgttagaatctcatgtgctgcctcaggatatacttttacaaactatggaatgaattgggtgaagcaggcacctgggcactgcgaccttagtcggaacacagaggtcgcccttattactacca

SEQ ID NO: 793 - DNA, 186 bp, Artificial
  Synthesized sequence
ccctttaatcagatgcgtcgataggattagctgatgggccgcagtgtgggtgaagcaggcacctgggaagggcctggagtggatgggttggattaacacttatacaggcgaatcaacatatgccgactcctttaagggcccactgcgaccttagtcggaacacagaggtcgcccttattactacca

SEQ ID NO: 794 - DNA, 182 bp, Artificial
  Synthesized sequence
ccctttaatcagatgcgtcgataggattagctgatgggccgcagtgatatgccgactcctttaagggccggttcaccttttctctcgacacttccgccagcgccgcctacctgcaaatcaacagcctgagggccgacactgcgaccttagtcggaacacagaggtcgcccttattactacca

SEQ ID NO: 795 - DNA, 187 bp, Artificial
  Synthesized sequence
ccctttaatcagatgcgtcgataggattagctgatgggccgcagtgtcaacagcctgagggccgaagatactgccgtgtattattgcgcaagatttgctattaagggggactactggggtcaagggaccctgctgacagtgcactgcgaccttagtcggaacacagaggtcgcccttattactacca

SEQ ID NO: 796 - DNA, 188 bp, Artificial
  Synthesized sequence
ccctttaatcagatgcgtcgataggattagctgatgggccgcagtgcaagggaccctgctgacagtgtccagcggcgggagcggcggttccggcggagcttccggagccgggtccggcggaggggatattcagatgacccagcactgcgaccttagtcggaacacagaggtcgcccttattactacca

SEQ ID NO: 797 - DNA, 187 bp, Artificial
  Synthesized sequence
ccctttaatcagatgcgtcgataggattagctgatgggccgcagtgcggaggggatattcagatgacccagtcacccagcagcctctctgcatctgtgggggacagggtgaccatcacctgtagatcaacaaaatctctgccactgcgaccttagtcggaacacagaggtcgcccttattactacca

SEQ ID NO: 798 - DNA, 185 bp, Artificial
  Synthesized sequence
ccctttaatcagatgcgtcgataggattagctgatgggccgcagtgtcacctgtagatcaacaaaatctctgctgcatagcaacggaatcacttacctgtactggtatcagcagaagcctggcaaagccccaaaactgccactgcgaccttagtcggaacacagaggtcgcccttattactacca

SEQ ID NO: 799 - DNA, 187 bp, Artificial
  Synthesized sequence
ccctttaatcagatgcgtcgataggattagctgatgggccgcagtgcctggcaaagccccaaaactgctgatctatcagatgtccaatctcgcatctggcgtcccatctaggtttagctcctccggctccggtacagacttcactgcgaccttagtcggaacacagaggtcgcccttattactacca

SEQ ID NO: 800 - DNA, 187 bp, Artificial
  Synthesized sequence
ccctttaatcagatgcgtcgataggattagctgatgggccgcagtgtccggctccggtacagacttcaccctgaccatatcaagcctgcagccagaggactttgccacttactattgcgctcagaatctcgaaatccctagcactgcgaccttagtcggaacacagaggtcgcccttattactacca

SEQ ID NO: 801 - DNA, 179 bp, Artificial
  Synthesized sequence
ccctttaatcagatgcgtcgataggattagctgatgggccgcagtggcgctcagaatctcgaaatccctaggacatttggacagggcacaaaggtcgaactgaaagggcccgcctagcaaccaacagtatgttcactgcgaccttagtcggaacacagaggtcgcccttattactacca

SEQ ID NO: 802 - DNA, 186 bp, Artificial
  Synthesized sequence
ccctttaatcagatgcgtcgtgagattcgggactattcgggcagtgtttcgcgtgagtggttcatataggcccagccggccaggcgcgaggttcaactcgtccaatctggccctgggctcgtccagcccgggggatccgtcactgcggtcggagtctaacaacagaggtcgcccttattactacca

SEQ ID NO: 803 - DNA, 184 bp, Artificial
  Synthesized sequence
ccctttaatcagatgcgtcgtgagattcgggactattcgggcagtgccagcccgggggatccgtccgcatctcctgcgccgcctctggctataccttcactaattatggcatgaactgggttaaacaggccccaggcacactgcggtcggagtctaacaacagaggtcgcccttattactacca

SEQ ID NO: 804 - DNA, 187 bp, Artificial
  Synthesized sequence
ccctttaatcagatgcgtcgtgagattcgggactattcgggcagtggggttaaacaggccccaggcaaaggtctggagtggatgggctggattaatacctataccggcgagtccacatacgccgatagctttaaggggaggcactgcggtcggagtctaacaacagaggtcgcccttattactacca

SEQ ID NO: 805 - DNA, 187 bp, Artificial
  Synthesized sequence
ccctttaatcagatgcgtcgtgagattcgggactattcgggcagtgacgccgatagctttaaggggaggttcactttcagcctcgataccagcgcttcagcagcatacctgcagattaactctctgcgcgccgaagatacccactgcggtcggagtctaacaacagaggtcgcccttattactacca

SEQ ID NO: 806 - DNA, 187 bp, Artificial
  Synthesized sequence
ccctttaatcagatgcgtcgtgagattcgggactattcgggcagtgctctgcgcgccgaagataccgctgtctactattgcgcccggttcgctattaagggggattactgggggcagggcacactcctgaccgtttcaagccactgcggtcggagtctaacaacagaggtcgcccttattactacca

SEQ ID NO: 807 - DNA, 187 bp, Artificial
  Synthesized sequence
ccctttaatcagatgcgtcgtgagattcgggactattcgggcagtgggcacactcctgaccgtttcaagcggcgggtccgccggctccggctcatctggcggggcatctgggagcggaggggacatacaaatgacacagtccactgcggtcggagtctaacaacagaggtcgcccttattactacca

SEQ ID NO: 808 - DNA, 187 bp, Artificial
  Synthesized sequence
ccctttaatcagatgcgtcgtgagattcgggactattcgggcagtggaggggacatacaaatgacacagtctccaagctctctgagcgcttctgtgggggatcgcgtcaccattacatgcagatccacaaaatccctgctgcactgcggtcggagtctaacaacagaggtcgcccttattactacca

SEQ ID NO: 809 - DNA, 187 bp, Artificial
  Synthesized sequence
ccctttaatcagatgcgtcgtgagattcgggactattcgggcagtgtgcagatccacaaaatccctgctgcatagcaatggcattacttatctgtattggtaccagcagaaacctggcaaagctcccaaactgctgatatacactgcggtcggagtctaacaacagaggtcgcccttattactacca

SEQ ID NO: 810 - DNA, 187 bp, Artificial
  Synthesized sequence
ccctttaatcagatgcgtcgtgagattcgggactattcgggcagtgcaaagctcccaaactgctgatataccagatgtccaatctggcctccggtgttcccagcagattctcaagctccggcagcgggacagactttactccactgcggtcggagtctaacaacagaggtcgcccttattactacca

SEQ ID NO: 811 - DNA, 187 bp, Artificial
  Synthesized sequence
ccctttaatcagatgcgtcgtgagattcgggactattcgggcagtgggcagcgggacagactttactctgaccatcagcagcctgcagcccgaggatttcgccacttactactgcgctcagaacctggaaatcccaagaaccactgcggtcggagtctaacaacagaggtcgcccttattactacca

SEQ ID NO: 812 - DNA, 175 bp, Artificial
  Synthesized sequence
ccctttaatcagatgcgtcgtgagattcgggactattcgggcagtgtcagaacctggaaatcccaagaacatttggccagggcactaaggttgaactgaaggggcccaacggcggaatccagtatatttcactgcggtcggagtctaacaacagaggtcgcccttattactacca

SEQ ID NO: 813 - DNA, 187 bp, Artificial
  Synthesized sequence
ccctttaatcagatgcgtcgttggttagtacacgggactcgcagtgttcaatagatacccacccgtcaggcccagccggccaggcgcgaggtgcagctggttgagtctggtgggaaactgctcaagcccggaggctcactgcactgcagtcccaagttcagacgtacggtcgcccttattactacca

SEQ ID NO: 814 - DNA, 185 bp, Artificial
  Synthesized sequence
ccctttaatcagatgcgtcgttggttagtacacgggactcgcagtgcaagcccggaggctcactgaagctgtcttgtgctgcttctggctttaccttcagcagcttcgcaatgtcttggtttcggcaaagcccagagaacactgcagtcccaagttcagacgtacggtcgcccttattactacca

SEQ ID NO: 815 - DNA, 187 bp, Artificial
  Synthesized sequence
ccctttaatcagatgcgtcgttggttagtacacgggactcgcagtgggtttcggcaaagcccagagaagcgcctggagtgggttgccgagatatcttctggagggtcatacacctactaccccgacactgttacaggtcggcactgcagtcccaagttcagacgtacggtcgcccttattactacca

SEQ ID NO: 816 - DNA, 189 bp, Artificial
  Synthesized sequence
ccctttaatcagatgcgtcgttggttagtacacgggactcgcagtgaccccgacactgttacaggtcggttcaccatctccagggataatgccaagaataccctgtatctggagatgtcttctctcaggtcagaagataccgccactgcagtcccaagttcagacgtacggtcgcccttattactacca

SEQ ID NO: 817 - DNA, 187 bp, Artificial
  Synthesized sequence
ccctttaatcagatgcgtcgttggttagtacacgggactcgcagtgtcttctctcaggtcagaagataccgctatgtactattgcgctagaggtctctggggttattatgcactcgattactggggccagggtactagcgtcactgcagtcccaagttcagacgtacggtcgcccttattactacca

SEQ ID NO: 818 - DNA, 187 bp, Artificial
  Synthesized sequence
ccctttaatcagatgcgtcgttggttagtacacgggactcgcagtgtggggccagggtactagcgtcacagtgtcctctggtggggccggctctggagccgggagcgggtcaagcggagccggatctggccagattgtcctcactgcagtcccaagttcagacgtacggtcgcccttattactacca

SEQ ID NO: 819 - DNA, 187 bp, Artificial
  Synthesized sequence
ccctttaatcagatgcgtcgttggttagtacacgggactcgcagtggccggatctggccagattgtcctcatccagtcccccgccatcatgtctgcttctccaggagagaaggtcaccatgacatgttccgcatcatcctccactgcagtcccaagttcagacgtacggtcgcccttattactacca

SEQ ID NO: 820 - DNA, 187 bp, Artificial
  Synthesized sequence
ccctttaatcagatgcgtcgttggttagtacacgggactcgcagtgcatgacatgttccgcatcatcctccgtttcttacatgtattggtatcagcagaagccaggctctagcccacgcctgctgatctatgacacttctacactgcagtcccaagttcagacgtacggtcgcccttattactacca

SEQ ID NO: 821 - DNA, 190 bp, Artificial
  Synthesized sequence
ccctttaatcagatgcgtcgttggttagtacacgggactcgcagtgcgcctgctgatctatgacacttctaacctcgcctccggagtgcccgtgcgcttttccggctcaggcagcggaacatcatatagcctgaccataagccgcactgcagtcccaagttcagacgtacggtcgcccttattactacca

SEQ ID NO: 822 - DNA, 187 bp, Artificial
  Synthesized sequence
ccctttaatcagatgcgtcgttggttagtacacgggactcgcagtgaacatcatatagcctgaccataagccgcatggaagccgaggatgccgcaacctattattgtcaacagtggtcagggtatccctacacattcggggcactgcagtcccaagttcagacgtacggtcgcccttattactacca

SEQ ID NO: 823 - DNA, 167 bp, Artificial
  Synthesized sequence
ccctttaatcagatgcgtcgttggttagtacacgggactcgcagtgcagggtatccctacacattcgggggaggcaccaaactggaaattaaggggcccagtgccaagggttcataagtttcactgcagtcccaagttcagacgtacggtcgcccttattactacca

SEQ ID NO: 824 - DNA, 187 bp, Artificial
  Synthesized sequence
ccctttaatcagatgcgtcgatttgtgtatcgaggctcgtgcagtgttatatatccgccgttgtacgtggcccagccggccaggcgccaagtgcagctggttcagtccggggccgaagtcaagaagcctgggtctagcgtgcactgcggttaaacaatcgcgtgtctggtcgcccttattactacca

SEQ ID NO: 825 - DNA, 190 bp, Artificial
  Synthesized sequence
ccctttaatcagatgcgtcgatttgtgtatcgaggctcgtgcagtgaagaagcctgggtctagcgtgaaggtctcttgcaaagccagcgggggaactttcaaccggtatactgttaactgggtgcggcaagctcctggccagggcactgcggttaaacaatcgcgtgtctggtcgcccttattactacca

SEQ ID NO: 826 - DNA, 190 bp, Artificial
  Synthesized sequence
ccctttaatcagatgcgtcgatttgtgtatcgaggctcgtgcagtgcggcaagctcctggccagggactggagtggatggggggaatcatccccatatttggaaccgctaactatgcacagcgcttccagggcagactgactatcactgcggttaaacaatcgcgtgtctggtcgcccttattactacca

SEQ ID NO: 827 - DNA, 190 bp, Artificial
  Synthesized sequence
ccctttaatcagatgcgtcgatttgtgtatcgaggctcgtgcagtggcttccagggcagactgactataaccgcagatgagtccacctcaaccgcctacatggagctgtcctctctgcggtccgacgatacagccgtgtactttcactgcggttaaacaatcgcgtgtctggtcgcccttattactacca

SEQ ID NO: 828 - DNA, 190 bp, Artificial
  Synthesized sequence
ccctttaatcagatgcgtcgatttgtgtatcgaggctcgtgcagtgccgacgatacagccgtgtacttttgcgcccgggagaacctggacaactctggcacttactattacttcagcggctggttcgacccttggggacaaggccactgcggttaaacaatcgcgtgtctggtcgcccttattactacca

SEQ ID NO: 829 - DNA, 190 bp, Artificial
  Synthesized sequence
ccctttaatcagatgcgtcgatttgtgtatcgaggctcgtgcagtgttcgacccttggggacaaggcaccagcgtcacagtctcatctggcggttctggggggagcggcggcgcttctggggccggaagcggtggcggtcagagcactgcggttaaacaatcgcgtgtctggtcgcccttattactacca

SEQ ID NO: 830 - DNA, 188 bp, Artificial
  Synthesized sequence
ccctttaatcagatgcgtcgatttgtgtatcgaggctcgtgcagtgaagcggtggcggtcagagcgcactgacccagcctcgcagcgtctccggctcccctgggcagagcgtgacaatatcttgtacaggcacctcctccgacactgcggttaaacaatcgcgtgtctggtcgcccttattactacca

SEQ ID NO: 831 - DNA, 190 bp, Artificial
  Synthesized sequence
ccctttaatcagatgcgtcgatttgtgtatcgaggctcgtgcagtgcttgtacaggcacctcctccgatatcggggggtataatttcgtgtcatggtaccagcaacatcccggcaaagccccaaagctgatgatctacgacgcccactgcggttaaacaatcgcgtgtctggtcgcccttattactacca

SEQ ID NO: 832 - DNA, 193 bp, Artificial
  Synthesized sequence
ccctttaatcagatgcgtcgatttgtgtatcgaggctcgtgcagtgccaaagctgatgatctacgacgccactaagaggccttccggggtgcccgataggttcagcgggagcaaatctggtaatactgcctcactgactatatcaggcactgcggttaaacaatcgcgtgtctggtcgcccttattactacca

SEQ ID NO: 833 - DNA, 187 bp, Artificial
  Synthesized sequence
ccctttaatcagatgcgtcgatttgtgtatcgaggctcgtgcagtgtaatactgcctcactgactatatcaggcctgcaggcagaagacgaggcagattattactgctgttcttacgccggtgactacacacctggtgtggcactgcggttaaacaatcgcgtgtctggtcgcccttattactacca

SEQ ID NO: 834 - DNA, 174 bp, Artificial
  Synthesized sequence
ccctttaatcagatgcgtcgatttgtgtatcgaggctcgtgcagtgggtgactacacacctggtgtggtgtttgggggcggcaccaagctgactgtgctggggcccaccgaacggcatacatctatttcactgcggttaaacaatcgcgtgtctggtcgcccttattactacca

SEQ ID NO: 835 - DNA, 186 bp, Artificial
  Synthesized sequence
ccctttaatcagatgcgtcgatcgttccccatcacattctgcagtgttcgagagtctcccacgatatcggcccagccggccaggcgccaggtccagctggtcgagtctggcggaggcgccgtgcagcccgggaggtccctcactgctaagtgctcaaaacgaacggggtcgcccttattactacca

SEQ ID NO: 836 - DNA, 187 bp, Artificial
  Synthesized sequence
ccctttaatcagatgcgtcgatcgttccccatcacattctgcagtggcagcccgggaggtccctgagactgtcttgcgctgcttcaggtttcactttttcttcctacggcatgcactgggtccgccaagctcctggaaaggcactgctaagtgctcaaaacgaacggggtcgcccttattactacca

SEQ ID NO: 837 - DNA, 187 bp, Artificial
  Synthesized sequence
ccctttaatcagatgcgtcgatcgttccccatcacattctgcagtgtccgccaagctcctggaaagggactggaatgggtcgccgtcatactgtacgacgggagcgacaagttttatgccgattcagtgaagggtcggtttcactgctaagtgctcaaaacgaacggggtcgcccttattactacca

SEQ ID NO: 838 - DNA, 187 bp, Artificial
  Synthesized sequence
ccctttaatcagatgcgtcgatcgttccccatcacattctgcagtgccgattcagtgaagggtcggtttactatttcacgcgataattccaagaacacactgtatctgcagatgaattccctgcgggctgaagatacagcccactgctaagtgctcaaaacgaacggggtcgcccttattactacca

SEQ ID NO: 839 - DNA, 187 bp, Artificial
  Synthesized sequence
ccctttaatcagatgcgtcgatcgttccccatcacattctgcagtgcctgcgggctgaagatacagccgtgtactactgtgcaaaagtggccgtggcagggactcactttgactattggggccaggggactctggtgactgcactgctaagtgctcaaaacgaacggggtcgcccttattactacca

SEQ ID NO: 840 - DNA, 187 bp, Artificial
  Synthesized sequences
ccctttaatcagatgcgtcgatcgttccccatcacattctgcagtggccaggggactctggtgactgtgtcctctgcaggcggttccgccggctctggctccagcgggggcgcttcaggctccgggggcgatatccaaatgcactgctaagtgctcaaaacgaacggggtcgcccttattactacca

SEQ ID NO: 841 - DNA, 183 bp, Artificial
  Synthesized sequence
ccctttaatcagatgcgtcgatcgttccccatcacattctgcagtgtccgggggcgatatccaaatgacccaaagcccatcctcactctccgcctctgttggcgatagagtcactattacctgcagggcctctcaggcactgctaagtgctcaaaacgaacggggtcgcccttattactacca

SEQ ID NO: 842 - DNA, 187 bp, Artificial
  Synthesized sequence
ccctttaatcagatgcgtcgatcgttccccatcacattctgcagtgtacctgcagggcctctcaggggatccgcaatgatctcggatggtaccagcagaaacccggaaaagctccaaaactgctgatatacgcagcttcttcactgctaagtgctcaaaacgaacggggtcgcccttattactacca

SEQ ID NO: 843 - DNA, 190 bp, Artificial
  Synthesized sequence
ccctttaatcagatgcgtcgatcgttccccatcacattctgcagtgaactgctgatatacgcagcttcttctctgcagtccggggtcccctcccggttctccggtagcggttctggaaccgactttacactgactatatcctctcactgctaagtgctcaaaacgaacggggtcgcccttattactacca

SEQ ID NO: 844 - DNA, 185 bp, Artificial
  Synthesized sequence
ccctttaatcagatgcgtcgatcgttccccatcacattctgcagtgaccgactttacactgactatatcctctctccagcctgaagacttcgctacatattactgccagcagctgaacagctaccctcccacattcggccactgctaagtgctcaaaacgaacggggtcgcccttattactacca

SEQ ID NO: 845 - DNA, 166 bp, Artificial
  Synthesized sequence
ccctttaatcagatgcgtcgatcgttccccatcacattctgcagtgcagctaccctcccacattcggcggcggtactaaggtggaaatcaaagggccccaaagtgcggaaaacagagattcactgctaagtgctcaaaacgaacggggtcgcccttattactacca

SEQ ID NO: 846 - DNA, 184 bp, Artificial
  Synthesized sequence
ccctttaatcagatgcgtcgattaccatgttatcgggcgagcagtgttattcagttggtcttacgggtggcccagccggccaggcgcgaagttcagctcgtggagtctggcggaggcgtggtccaacctggcaggtcccactgcaatcttgcgttccctaacctggtcgcccttattactacca

SEQ ID NO: 847 - DNA, 188 bp, Artificial
  Synthesized sequence
ccctttaatcagatgcgtcgattaccatgttatcgggcgagcagtgtggtccaacctggcaggtccctgaggctgtcttgttctgccagcggatttacattttccgggtacggactgtcctgggtcagacaggctccagggacactgcaatcttgcgttccctaacctggtcgcccttattactacca

SEQ ID NO: 848 - DNA, 184 bp, Artificial
  Synthesized sequence
ccctttaatcagatgcgtcgattaccatgttatcgggcgagcagtggggtcagacaggctccagggaaaggcctcgaatgggtggcaatgatctctagcggaggctcatacacctattacgccgactccgtcaaggggcactgcaatcttgcgttccctaacctggtcgcccttattactacca

SEQ ID NO: 849 - DNA, 183 bp, Artificial
  Synthesized sequence
ccctttaatcagatgcgtcgattaccatgttatcgggcgagcagtgacgccgactccgtcaaggggcgcttcgccatcagcagagataatgcaaagaatactctcttcctccagatggattctctccggcccgaggacactgcaatcttgcgttccctaacctggtcgcccttattactacca

SEQ ID NO: 850 - DNA, 188 bp, Artificial
  Synthesized sequence
ccctttaatcagatgcgtcgattaccatgttatcgggcgagcagtgattctctccggcccgaggacaccggtgtgtacttctgtgctcgccatggggatgacccagcctggtttgcttactggggccagggaactcctgtgacactgcaatcttgcgttccctaacctggtcgcccttattactacca

SEQ ID NO: 851 - DNA, 188 bp, Artificial
  Synthesized sequence
ccctttaatcagatgcgtcgattaccatgttatcgggcgagcagtggggccagggaactcctgtgaccgtttctagcgggggggctggcagcggggccggttcaggttcttccggcgccggctccggggacatccagctcaccactgcaatcttgcgttccctaacctggtcgcccttattactacca

SEQ ID NO: 852 - DNA, 188 bp, Artificial
  Synthesized sequence
ccctttaatcagatgcgtcgattaccatgttatcgggcgagcagtgtccggggacatccagctcactcagagcccatcttcactgtcagcatccgtcggagatagagtgactataacctgttcagtgtcctcatcaatcagccactgcaatcttgcgttccctaacctggtcgcccttattactacca

SEQ ID NO: 853 - DNA, 188 bp, Artificial
  Synthesized sequence
ccctttaatcagatgcgtcgattaccatgttatcgggcgagcagtgctgttcagtgtcctcatcaatcagctccaacaatctgcactggtaccagcagaaaccaggaaaggcaccaaaaccctggatatacggcacctcaaacactgcaatcttgcgttccctaacctggtcgcccttattactacca

SEQ ID NO: 854 - DNA, 188 bp, Artificial
  Synthesized sequence
ccctttaatcagatgcgtcgattaccatgttatcgggcgagcagtgccctggatatacggcacctcaaatctggcttccggtgtgccttccagattctcagggagcggatccggcaccgactacacctttacaatcagctcccactgcaatcttgcgttccctaacctggtcgcccttattactacca

SEQ ID NO: 855 - DNA, 190 bp, Artificial
  Synthesized sequence
ccctttaatcagatgcgtcgattaccatgttatcgggcgagcagtgcgactacacctttacaatcagctccctgcagcccgaggacattgcaacatactactgtcaacagtggagctcctatccctatatgtacaccttcggaccactgcaatcttgcgttccctaacctggtcgcccttattactacca

SEQ ID NO: 856 - DNA, 169 bp, Artificial
  Synthesized sequence
ccctttaatcagatgcgtcgattaccatgttatcgggcgagcagtgctatccctatatgtacaccttcggacagggaacaaaggttgagattaaagggcccaccgggaaagacgaataactttcactgcaatcttgcgttccctaacctggtcgcccttattactacca

SEQ ID NO: 857 - DNA, 182 bp, Artificial
  Synthesized sequence
ccctttaatcagatgcgtcgtcggtggatatgacgtaaccgcagtgttggattgcaacgtcaggaaatggcccagccggccaggcgcgaggtgcagctcgtcgagtccggaggcggcctggttcagcctggcgggtcactgcagataacgagcacagtctggggtcgcccttattactacca

SEQ ID NO: 858 - DNA, 187 bp, Artificial
  Synhesized sequence
ccctttaatcagatgcgtcgtcggtggatatgacgtaaccgcagtgctggttcagcctggcgggtctctccgcctgtcctgcgccgcctccggattcgactttagcagatactggatgtcctgggtgagacaggctcctggcactgcagataacgagcacagtctggggtcgcccttattactacca

SEQ ID NO: 859 - DNA, 186 bp, Artificial
  Synthesized sequence
ccctttaatcagatgcgtcgtcggtggatatgacgtaaccgcagtgctgggtgagacaggctcctggaaaaggactcgaatggatcggggagatcaaccccgattcttccaccatcaactacgcacctagcctgaaagatcactgcagataacgagcacagtctggggtcgcccttattactacca

SEQ ID NO: 860 - DNA, 187 bp, Artificial
  Synthesized sequence
ccctttaatcagatgcgtcgtcggtggatatgacgtaaccgcagtgactacgcacctagcctgaaagataaattcatcatttccagagacaatgccaaaaattcactgtacctccaaatgaacagcctgagagctgaggatcactgcagataacgagcacagtctggggtcgcccttattactacca

SEQ ID NO: 861 - DNA, 187 bp, Artificial
  Synthesized sequence
ccctttaatcagatgcgtcgtcggtggatatgacgtaaccgcagtgaacagcctgagagctgaggatactgctgtctactactgcgctaggcccgatgggaattactggtacttcgatgtgtgggggcagggcactctggtcactgcagataacgagcacagtctggggtcgcccttattactacca

SEQ ID NO: 862 - DNA, 187 bp, Artificial
  Synthesized sequence
ccctttaatcagatgcgtcgtcggtggatatgacgtaaccgcagtggggggcagggcactctggttaccgtgtcatcaggtggctccggagggtccggcggcgcaagcggagccggatccggcggaggagacatccagatgcactgcagataacgagcacagtctggggtcgcccttattactacca

SEQ ID NO: 863 - DNA, 187 bp, Artificial
  Synthesized sequence
ccctttaatcagatgcgtcgtcggtggatatgacgtaaccgcagtgcggcggaggagacatccagatgacacagtctccatccagcctcagcgcctccgttggcgatcgggtgacaatcacctgcaaggcctcacaggacgcactgcagataacgagcacagtctggggtcgcccttattactacca

SEQ ID NO: 864 - DNA, 187 bp, Artificial
  Synthesized sequence
ccctttaatcagatgcgtcgtcggtggatatgacgtaaccgcagtgctgcaaggcctcacaggacgtcggaatcgccgttgcttggtatcaacaaaagcccgggaaggtccccaagctgctgatttattgggcctctacaccactgcagataacgagcacagtctggggtcgcccttattactacca

SEQ ID NO: 865 - DNA, 187 bp, Artificial
  Synthesized sequence
ccctttaatcagatgcgtcgtcggtggatatgacgtaaccgcagtgctgctgatttattgggcctctacacggcacacaggtgttccagatcgcttctctggtagcggctccggaaccgactttactctgactatatcttccactgcagataacgagcacagtctggggtcgcccttattactacca

SEQ ID NO: 866 - DNA, 187 bp, Artificial
  Synthesized sequence
ccctttaatcagatgcgtcgtcggtggatatgacgtaaccgcagtggaaccgactttactctgactatatcttctctgcagcccgaggatgtggccacttactactgtcagcaatatagctcctacccatacacttttggccactgcagataacgagcacagtctggggtcgcccttattactacca

SEQ ID NO: 867 - DNA, 169 bp, Artificial
  Synthesized sequence
ccctttaatcagatgcgtcgtcggtggatatgacgtaaccgcagtgtagctcctacccatacacttttggccaggggacaaaagtggagatcaaagggcccgcttcgtggagattcctgtattcactgcagataacgagcacagtctggggtcgcccttattactacca

SEQ ID NO: 868 - DNA, 187 bp, Artificial
  Synthesized sequence
ccctttaatcagatgcgtcgggtcagatggtttacatgcggcagtgttgaatgttgcagactggaaggggcccagccggccaggcgccaggtgcagctgcaagaatcagggccaggactcgtcaaaccctctcaaacactgcactgcatcgcggatagagaacaactggtcgcccttattactacca

SEQ ID NO: 869 - DNA, 187 bp, Artificial
  Synthesized sequence
ccctttaatcagatgcgtcgggtcagatggtttacatgcggcagtgctcgtcaaaccctctcaaacactgtctctgacttgtaccgtgtctgggggctccatctcatccggggattactactggtcatggatcaggcaacccactgcatcgcggatagagaacaactggtcgcccttattactacca

SEQ ID NO: 870 - DNA, 187 bp, Artificial
  Synthesized sequence
ccctttaatcagatgcgtcgggtcagatggtttacatgcggcagtgtactggtcatggatcaggcaaccacctggcaaaggtctggagtggattggctatatctactactctgggtcaaccgattataacccaagcctcaacactgcatcgcggatagagaacaactggtcgcccttattactacca

SEQ ID NO: 871 - DNA, 187 bp, Artificial
  Synthesized sequence
ccctttaatcagatgcgtcgggtcagatggtttacatgcggcagtgaaccgattataacccaagcctcaagtctcgggttacaatgagcgtggatactagcaagaatcaattctcactcaaggtgaactctgttactgccgcactgcatcgcggatagagaacaactggtcgcccttattactacca

SEQ ID NO: 872 - DNA, 188 bp, Artificial
  Synthesized sequence
ccctttaatcagatgcgtcgggtcagatggtttacatgcggcagtgtcaaggtgaactctgttactgccgctgacaccgccgtgtactattgcgctcgggtctctatcttcggtgtggggacctttgactattggggtcaagcactgcatcgcggatagagaacaactggtcgcccttattactacca

SEQ ID NO: 873 - DNA, 180 bp, Artificial
  Synthesized sequence
ccctttaatcagatgcgtcgggtcagatggtttacatgcggcagtggggacctttgactattggggtcaaggaacactggtcactgtttcaagcggcggctctgcagggtcaggctcatccggaggcgcctccgcactgcatcgcggatagagaacaactggtcgcccttattactacca

SEQ ID NO: 874 - DNA, 186 bp, Artificial
  Synthesized sequence
ccctttaatcagatgcgtcgggtcagatggtttacatgcggcagtgcatccggaggcgcctccggctctggcggcgaaatagtgatgactcagtcaccagctactctgtccctctcccctggagagagggctacactctccactgcatcgcggatagagaacaactggtcgcccttattactacca

SEQ ID NO: 875 - DNA, 182 bp, Artificial
  Synthesized sequence
ccctttaatcagatgcgtcgggtcagatggtttacatgcggcagtgcctggagagagggctacactctcttgccgcgcctcacagtctgtgagcagctacctcgcttggtaccagcagaaaccaggtcaggccccccactgcatcgcggatagagaacaactggtcgcccttattactacca

SEQ ID NO: 876 - DNA, 185 bp, Artificial
  Synthesized sequence
ccctttaatcagatgcgtcgggtcagatggtttacatgcggcagtggaaaccaggtcaggccccccggctgctgatctatgacgctagcaatcgggctactggcatccccgccagattttctggatctgggtcaggcaccactgcatcgcggatagagaacaactggtcgcccttattactacca

SEQ ID NO: 877 - DNA, 188 bp, Artificial
  Synthesized sequence
ccctttaatcagatgcgtcgggtcagatggtttacatgcggcagtgtttctggatctgggtcaggcaccgacttcacactgactataagctcactggagcccgaagacttcgccgtgtattactgccatcagtatggaagcacactgcatcgcggatagagaacaactggtcgcccttattactacca

SEQ ID NO: 878 - DNA, 186 bp, Artificial
  Synthesized sequence
ccctttaatcagatgcgtcgggtcagatggtttacatgcggcagtgtattactgccatcagtatggaagcacccccctgacctttgggggtggtaccaaagccgagattaaggggcccatctagtaacaagcccgaggttcactgcatcgcggatagagaacaactggtcgcccttattactacca

SEQ ID NO: 879 - DNA, 188 bp, Artificial
  Synthesized sequence
ccctttaatcagatgcgtcgtctcgttcgaaaatcatcgcgcagtgttgtccatgaatacaacaccggggcccagccggccaggcgcgaggttcagctcctggagtccgggggcggactggtgcagcccgggggctcactgacactgcgtcaccggcgagatttaatcggtcgcccttattactacca

SEQ ID NO: 880 - DNA, 187 bp, Artificial
  Synthesized sequence
ccctttaatcagatgcgtcgtctcgttcgaaaatcatcgcgcagtgagcccgggggctcactgaggctgagctgcacagcctctggcttcacatttagctcctacgccatgaattgggtgagacaagcccctggaaaggggcactgcgtcaccggcgagatttaatcggtcgcccttattactacca

SEQ ID NO: 881 - DNA, 189 bp, Artificial
  Synthesized sequence
ccctttaatcagatgcgtcgtctcgttcgaaaatcatcgcgcagtggagacaagcccctggaaaggggctggagtgggtgtctgctatttcaggctcaggggggacaaccttttatgccgacagcgtgaagggcaggttcacccactgcgtcaccggcgagatttaatcggtcgcccttattactacca

SEQ ID NO: 882 - DNA, 190 bp, Artificial
  Synthesized sequence
ccctttaatcagatgcgtcgtctcgttcgaaaatcatcgcgcagtgagcgtgaagggcaggttcaccatttcacgcgataactcacgcactaccctctatctgcagatgaattccctgcgggcagaagacacagccgtctattacactgcgtcaccggcgagatttaatcggtcgcccttattactacca

SEQ ID NO: 883 - DNA, 186 bp, Artificial
  Synthesized sequence
ccctttaatcagatgcgtcgtctcgttcgaaaatcatcgcgcagtgggcagaagacacagccgtctattattgtgcaaaagacctgggatggtctgactcatattattattattatgggatggatgtttgggggcaggggcactgcgtcaccggcgagatttaatcggtcgcccttattactacca

SEQ ID NO: 884 - DNA, 184 bp, Artificial
  Synthesized sequence
ccctttaatcagatgcgtcgtctcgttcgaaaatcatcgcgcagtgatggatgtttgggggcaggggaccaccgtgaccgtcagcagcggcggggcaggatctggggccgggtctggctcatcaggggccggttctggcactgcgtcaccggcgagatttaatcggtcgcccttattactacca

SEQ ID NO: 885 - DNA, 189 bp, Artificial
  Synthesized sequence
ccctttaatcagatgcgtcgtctcgttcgaaaatcatcgcgcagtgcatcaggggccggttctggggatatacagatgacccagttcccatcatctctctcagcctctgtcggggatagggttaccattacttgcagagccagcactgcgtcaccggcgagatttaatcggtcgcccttattactacca

SEQ ID NO: 886 - DNA, 188 bp, Artificial
  Synthesized sequence
ccctttaatcagatgcgtcgtctcgttcgaaaatcatcgcgcagtgggttaccattacttgcagagccagccagggaatcagaaatgatctgggctggtatcaacagaaaccaggtaaagcccccaagaggctcatctacgccactgcgtcaccggcgagatttaatcggtcgcccttattactacca

SEQ ID NO: 887 - DNA, 189 bp, Artificial
  Synthesized sequence
ccctttaatcagatgcgtcgtctcgttcgaaaatcatcgcgcagtggcccccaagaggctcatctacgccgcatcccgcctgcatcggggagtcccttcacgcttttccggctctggctcaggtaccgagttcactctcactacactgcgtcaccggcgagatttaatcggtcgcccttattactacca

SEQ ID NO: 888 - DNA, 192 bp, Artificial
  Synthesized sequence
ccctttaatcagatgcgtcgtctcgttcgaaaatcatcgcgcagtgcaggtaccgagttcactctcactatttccagcctccagccagaggattttgcaacctactactgcctgcaacataattcttatccctgttcatttggtcacactgcgtcaccggcgagatttaatcggtcgcccttattactacca

SEQ ID NO: 889 - DNA, 169 bp, Artificial
  Synthesized sequence
ccctttaatcagatgcgtcgtctcgttcgaaaatcatcgcgcagtgtaattcttatccctgttcatttggtcagggcacaaagctcgaaattaaggggcccagtacgttggacggaagaatttcactgcgtcaccggcgagatttaatcggtcgcccttattactacca